(19) United States
(12) Patent Application Publication
(10) Pub. No.: US 2025/0163047 A1
(43) Pub. Date: May 22, 2025

(54) HETEROCYCLIC COMPOUNDS, COMPOSITIONS THEREOF, AND METHODS OF TREATMENT THEREWITH

(71) Applicant: BeiGene, Ltd., Camana Bay (KY)

(72) Inventors: Guoliang Zhang, Beijing (CN); Jianzhuang Miao, Beijing (CN); Hanzi Sun, Beijing (CN); Zhikun Ni, Beijing (CN); Zhi Zhang, Beijing (CN); Ce Wang, Beijing (CN)

(73) Assignee: BeiGene, Ltd., Camana Bay (KY)

(21) Appl. No.: 18/796,700

(22) Filed: Aug. 7, 2024

Related U.S. Application Data
(63) Continuation of application No. PCT/CN2023/075346, filed on Feb. 10, 2023.

(30) Foreign Application Priority Data
Feb. 10, 2022 (WO) PCTCN2022075794
Apr. 28, 2022 (WO) PCTCN2022090094
Oct. 20, 2022 (WO) PCTCN2022126380
Jan. 10, 2023 (WO) PCTCN2023071744

(51) Int. Cl. C07D 471/04; A61K 31/435; A61K 31/4355; A61K 31/437; A61K 31/4545; A61K 31/5025; A61K 31/5377; C07D 401/14; C07D 487/04; C07D 491/04; C07D 495/04

(52) U.S. Cl. CPC C07D 471/04; A61K 31/435; A61K 31/4355; A61K 31/437; A61K 31/4545; A61K 31/5025; A61K 31/5377; C07D 401/14; C07D 487/04; C07D 491/04; C07D 495/04

(57) ABSTRACT

Provided herein are compounds having the following structure:

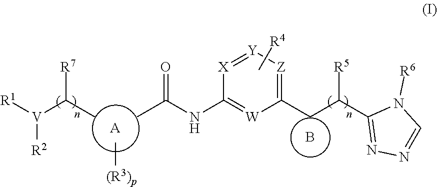

wherein the substituents are as defined herein, compositions comprising an effective amount of a compound, and methods for modulating activity of an immune cell.

### FIELD

Provided herein are compounds of Formula (I) for activating T cells, NK cells, and/or exhibiting antitumor activity, methods of using the compounds disclosed herein for treating cancer, and a pharmaceutical composition comprising the same.

### BACKGROUND

Casitas B-lineage lymphoma proto-oncogene b (Cbl-b) is one of the members of the Cbl family ubiquitin ligases. Cbl family proteins share similar structure features of a highly conserved tyrosine kinase binding (TKB) domain which recognizes the substrates and a C3HC4 RING (really interesting new gene) finger domain responsible for the E3 ubiquitin ligase activity (Tsygankov, A., Cbl proteins. 2008, New York: Nova Science Publishers, vi, 210 p). The first member of Cbl family in mammalian cells, c-Cbl (Cbl or RNF55), was identified as a proto-oncogenic protein in 1989 (Langdon, W. Y., et al., Proc Natl Acad Sci USA, 1989. 86(4): p. 1168-72), while Cbl-b (RNF56) and Cbl-c (Cbl-3 or RNF57) were subsequently cloned and characterized with high sequence homology with c-Cbl at N terminus (Keane, M. M., et al., Oncogene, 1995. 10(12): p. 2367-77; Kim, M., et al., Gene, 1999. 239(1): p. 145-54). Among the Cbl family, both c-Cbl and Cbl-b are well studied and ubiquitously expressed in mammalian tissues. On the other hand, Cbl-c is less known with weak E3 activity and shows restricted expression in epithelial cells (Swaminathan, G. and A. Y. Tsygankov, Journal of Cellular Physiology, 2006. 209(1): p. 21-43).

Cbl family members show similar autoinhibition and phosphorylation-induced activation mechanisms. Structure and biochemical studies revealed that inactive c-Cbl and Cbl-b adopt autoinhibitory conformations where RING domain position with low binding affinity with E2 ubiquitin-conjugating enzyme (UbcH5B). Tyrosine phosphorylation (Y371 of c-Cbl and Y363 of Cbl-b) induces a large conformational change to remove the masking of the RING domain from the TKB domain, forming a surface to enhance binding affinity of c-Cbl and Cbl-b to E2 (Kobashigawa, Y., et al., Proc Natl Acad Sci USA, 2011. 108(51): p. 20579-84; Dou, H., et al., Nat Struct Mol Biol, 2012. 19(2): p. 184-92). Upon activation, the structural rearrangement of c-Cbl and Cbl-b positions the E2 toward the substrates, and facilitates the poly- or mono-ubiquitin transfer to the lysine residues. The ubiquitylated membrane proteins could further bind to ESCRT complex internalize into the endosome, and attenuate receptor mediated cell signaling (Ma, K., et al., 2013. p. 219-244).

Among the three Cbl family members, Cbl-b was widely studied and emerged as an important intracellular checkpoint regulator in antitumor immunity. Cbl-b knockout mice showed strong rejection of EL4, EG7, TC-1, B16F10 syngeneic transplanting tumors, and ATM knockout or UVB-induced spontaneous tumors (Loeser, S., et al., J Exp Med, 2007. 204(4): p. 879-91; Chiang, J. Y., et al., J Clin Invest, 2007. 117(4): p. 1029-36; Paolino, M., et al., Nature, 2014. 507(7493): p. 508-12). CD8+ T cells and NK cells were found to play key roles in eliminating tumors in Cbl-b knockout mice, as depletion of either cell population abrogated the Cbl-b knockout induced tumor rejection (Loeser, S., et al., J Exp Med, 2007. 204(4): p. 879-91; Paolino, M., et al., Nature, 2014. 507(7493): p. 508-12). Cbl-b deficiency reduced T cell activation threshold and regulated co-stimulatory pathway to enhance TCR signaling (Fang, D. and Y. C. Liu, Nat Immunol, 2001. 2(9): p. 870-5; Bachmaier, K., et al., Nature, 2000. 403(6766): p. 211-6; Braun, M., et al., 2020. 53(4): p. 805-823 e15; Zhang, J., et al., 2002. 169(5): p. 2236-40). Cbl-b depletion or E3 inactivate mutation were shown to enhance NK cell function by blocking the ligand induced TAM receptors internalization (Paolino, M., et al., Nature, 2014. 507(7493): p. 508-12). The bone marrow-derived dendritic cells (BMDCs) and macrophages without Cbl-b expression in mice also exhibit stronger activation after stimulation, which might be also contribute to the Cbl-b deficiency induced tumor regression (Abe, T., et al., Diabetes, 2013. 62(6): p. 1957-69; Wallner, S., et al., PLoS One, 2013. 8(6): p. e65178.). Collectively, targeting Cbl-b is a potential novel immunotherapy against cancer.

Although some literatures showed that knockout both c-Cbl and Cbl-b could induce stronger T cell activation than c-Cbl or Cbl-b single knockout, c-Cbl may not be an optimal immune-oncology target since it functions as a tumor suppressor by downregulating activated receptor tyrosine kinases (RTK). In clinical, the heterozygous germline mutations (LOF) in CBL are related to Noonan syndrome, RASopathy and increase risks of acute myeloid leukemia, myeloproliferative neoplasms, and juvenile myelomonocytic leukemia (Grand, F. H., et al., Blood, 2009. 113(24): p. 6182-92; Becker, H., et al., Blood, 2014. 123(12): p. 1883-6; Loh, M. L., et al., Blood, 2009. 114(9): p. 1859-63; Martinelli, S., et al., Am J Hum Genet, 2010. 87(2): p. 250-7). In contrast to Cbl-b knockout, c-Cbl knockout altered the tumor microenvironment and promoted tumor growth in B16F10 and MC38 syngeneic models (Meyer, R. D., D. Husain, and N. Rahimi, Oncogene, 2011. 30(19): p. 2198-206; Lyle, C., et al., Sci Rep, 2019. 9(1): p. 20257). Knockout both c-Cbl and Cbl-b lead to embryonic lethality in mice. Hematopoietic c-Cbl and Cbl-b double knockout induced strong auto-immune diseases and early-onset lethal myeloproliferative disease (Naramura, M., et al., Nat Immunol, 2002. 3(12): p. 1192-9; Naramura, M., et al., Proc Natl Acad Sci USA, 2010. 107(37): p. 16274-9). Therefore, Cbl-b selective inhibition over c-Cbl should be achieved to avoid the potential tumor promoting effects and safety risks.

Although the existing anti-CTLA-4 and anti-PD-1 therapies have shown clear clinical benefits in a subset of patients with various tumor types, there are still unmet medical needs to develop novel immunotherapies to achieve robust and durable clinical anti-tumor efficacy. Pre-clinical data strongly suggest there is great potential of developing selective Cbl-b targeted therapies to improve antitumor immunity.

Citation or identification of any reference in this section is not to be construed as an admission that the reference is prior art to the present application.

### SUMMARY

Provided herein are compounds having the formula (I):

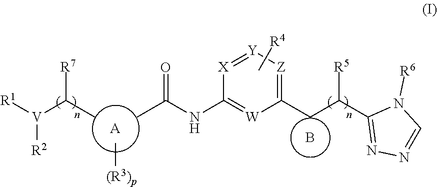

or a pharmaceutically acceptable salt, tautomer, stereoisomer, isotopologue, or enantiomer thereof,
wherein:
each of V, W, X, Y, and Z is, independently CH or N;
each of R¹ and R² is, independently, hydrogen, substituted or unsubstituted C₁₋₈ alkyl, substituted or unsubstituted cycloalkyl, substituted or unsubstituted non-aromatic heterocyclyl, substituted or unsubstituted saturated cycloalkylalkyl, substituted or unsubstituted non-aromatic heterocyclylalkyl, or R¹ and R² together with the atom which R¹ and R² connect to form a substituted or unsubstituted cycloalkyl or non-aromatic heterocyclyl;
R³ is hydrogen, halogen, —CN, hydroxyl, substituted or unsubstituted C₁₋₈ alkyl, or substituted or unsubstituted saturated cycloalkyl;
R⁴ is hydrogen, halogen, substituted or unsubstituted C₁₋₈ alkyl, or substituted or unsubstituted saturated cycloalkyl;
R⁵ is hydrogen, halogen, substituted or unsubstituted C₁₋₈ alkyl, or substituted or unsubstituted saturated cycloalkyl;
R⁶ is hydrogen, or substituted or unsubstituted C₁₋₈ alkyl;
R⁷ is hydrogen, deuterium, halogen, or substituted or unsubstituted C₁₋₈ alkyl or substituted or unsubstituted saturated cycloalkyl;
ring A is substituted or unsubstituted aryl, or substituted or unsubstituted heteroaryl;
moiety B is substituted or unsubstituted aryl, substituted or unsubstituted cycloalkyl, substituted or unsubstituted cycloalkylalkyl, a substituted or unsubstituted saturated spiro bicyclic ring, or substituted or unsubstituted non-aromatic heterocyclyl; and
each of m, n and p is, independently, 0, 1, or 2.

In one embodiment, the compound having formula (I) is a compound of formula (IIa):

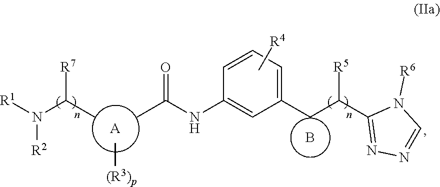

or a pharmaceutically acceptable salt, tautomer, stereoisomer, enantiomer, isotopologue, or prodrug thereof.

In one embodiment, the compound having formula (I) is a compound of formula (IIb):

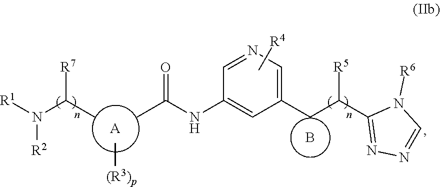

or a pharmaceutically acceptable salt, tautomer, stereoisomer, enantiomer, isotopologue, or prodrug thereof.

In one embodiment, the compound having formula (I) is a compound of formula (IIIa):

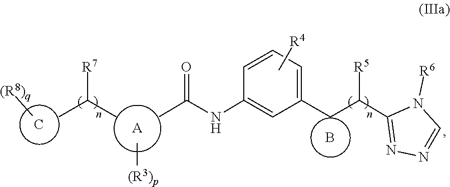

or a pharmaceutically acceptable salt, tautomer, stereoisomer, enantiomer, isotopologue, or prodrug thereof,
wherein moiety C is a substituted or unsubstituted saturated spiro bicyclic ring, substituted or unsubstituted non-aromatic heterocyclyl, or substituted or unsubstituted amino;
R⁸ is hydrogen, halogen, substituted or unsubstituted C₁₋₈ alkyl, substituted or unsubstituted C₁₋₈ alkoxyl, substituted or unsubstituted saturated cycloalkyl, or substituted or unsubstituted saturated cycloalkylalkyl; or two R⁸ together with the atom(s) which they connect to form a substituted or unsubstituted saturated cycloalkyl or substituted or unsubstituted saturated spiro cyclic ring; and
q is 0, 1, 2, or 3.

In one embodiment, the compound having formula (I) is a compound of formula (IIIb):

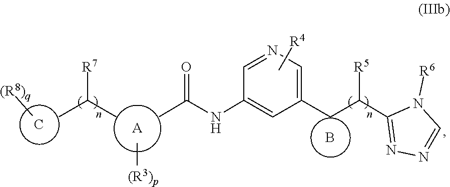

or a pharmaceutically acceptable salt, tautomer, stereoisomer, enantiomer, isotopologue, or prodrug thereof,
wherein moiety C is a substituted or unsubstituted saturated spiro bicyclic ring, substituted or unsubstituted non-aromatic heterocyclyl, or substituted or unsubstituted amino;
R⁸ is hydrogen, halogen, substituted or unsubstituted C₁₋₈ alkyl, substituted or unsubstituted C₁₋₈ alkoxyl, substituted or unsubstituted saturated cycloalkyl, or substituted or unsubstituted saturated cycloalkylalkyl; or two R⁸ together with the atom(s) which they connect to form a substituted or unsubstituted saturated cycloalkyl or substituted or unsubstituted saturated spiro cyclic ring; and
q is 0, 1, 2, or 3,
the other variables R³, R⁴, R⁵, R⁶, R⁷, ring A, moeity B, p and n are defined as in Formula (I).

Provided here is a pharmaceutical composition comprising an effective amount of a compound provided herein, or a pharmaceutically acceptable salt, tautomer, isotopologue, stereoisomer, or prodrug thereof, and a pharmaceutically acceptable carrier, excipient or vehicle.

Provided here is a method of modulating activity of an immune cell, comprising contacting said cell with an effective amount of a compound provided herein, or a pharmaceutically acceptable salt, tautomer, isotopologue, stereoisomer, or prodrug thereof.

Provided here is a method for the treatment or prevention of a cancer responsive to Cbl-b activity, the methods comprising administering to a subject in need thereof an effective amount of a compound provided herein. In one embodiment, the cancer is hematologic cancer. In one embodiment, the hematologic cancer is is lymphoma, leukemia, myeloma, or glioblastoma.

### DETAILED DESCRIPTION

### Definitions

The term “Cbl-b” as used herein refers to a Cbl-b protein. The term also includes naturally occurring variants of Cbl-b, including splice variants or allelic variants. The term also includes non-naturally occurring variants of Cbl-b, such as a recombinant Cbl-b protein or truncated variants thereof, which generally preserve the binding ability of naturally occurring Cbl-b or naturally occurring variants of Cbl-b (e.g., the ability to bind to an E2 enzyme).

As used herein, and in the specification and the accompanying claims, the indefinite articles “a” and “an” and the definite article “the” include plural as well as single referents, unless the context clearly indicates otherwise.

As used herein, and unless otherwise specified, the terms “about” and “approximately,” when used in connection with doses, amounts, or weight percents of ingredients of a composition or a dosage form, mean a dose, amount, or weight percent that is recognized by one of ordinary skill in the art to provide a pharmacological effect equivalent to that obtained from the specified dose, amount, or weight percent. In certain embodiments, the terms “about” and “approximately,” when used in this context, contemplate a dose, amount, or weight percent within 30%, within 20%, within 15%, within 10%, or within 5%, of the specified dose, amount, or weight percent.

As used herein, and unless otherwise specified, the terms “about” and “approximately,” when used in connection with a numeric value or range of values which is provided to characterize a particular solid form, e.g., a specific temperature or temperature range, such as, for example, that describes a melting, dehydration, desolvation, or glass transition temperature; a mass change, such as, for example, a mass change as a function of temperature or humidity; a solvent or water content, in terms of, for example, mass or a percentage; or a peak position, such as, for example, in analysis by, for example, IR or Raman spectroscopy or XRPD; indicate that the value or range of values may deviate to an extent deemed reasonable to one of ordinary skill in the art while still describing the solid form. Techniques for characterizing crystal forms and amorphous solids include, but are not limited to, thermal gravimetric analysis (TGA), differential scanning calorimetry (DSC), X-ray powder diffractometry (XRPD), single-crystal X-ray diffractometry, vibrational spectroscopy, e.g., infrared (IR) and Raman spectroscopy, solid-state and solution nuclear magnetic resonance (NMR) spectroscopy, optical microscopy, hot stage optical microscopy, scanning electron microscopy (SEM), electron crystallography and quantitative analysis, particle size analysis (PSA), surface area analysis, solubility studies, and dissolution studies. In certain embodiments, the terms “about” and “approximately,” when used in this context, indicate that the numeric value or range of values may vary within 30%, 20%, 15%, 10%, 9%, 8%, 7%, 6%, 5%, 4%, 3%, 2%, 1.5%, 1%, 0.5%, or 0.25% of the recited value or range of values. For example, in some embodiments, the value of an XRPD peak position may vary by up to ±0.2° 2θ (or ±0.2 degree 2θ) while still describing the particular XRPD peak.

An “alkyl” group is a saturated, partially saturated, or unsaturated straight chain or branched non-cyclic hydrocarbon having from 1 to 10 carbon atoms, typically from 1 to 8 carbons or, in some embodiments, from 1 to 6, 1 to 4, or 2 to 6 or carbon atoms. Representative alkyl groups include -methyl, -ethyl, -n-propyl, -n-butyl, -n-pentyl and -n-hexyl; while saturated branched alkyls include -isopropyl, -sec-butyl, -isobutyl, -tert-butyl, -isopentyl, -neopentyl, tert-pentyl, -2-methylpentyl, -3-methylpentyl, -4-methylpentyl, -2,3-dimethylbutyl and the like. Examples of unsaturated alkyl groups include, but are not limited to, vinyl, allyl, —CH═CH(CH₃), —CH═C(CH₃)₂, —C(CH₃)═CH₂, —C(CH₃)═CH(CH₃), —C(CH₂CH₃)═CH₂, —C≡CH, —C≡C(CH₃), —C≡C(CH₂CH₃), —CH₂C≡CH, —CH₂C≡C(CH₃) and —CH₂C≡C(CH₇CH₃), among others. An alkyl group can be substituted or unsubstituted. When the alkyl groups described herein are said to be “substituted,” they may be substituted with any substituent or substituents as those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amino; alkoxyamine; aralkoxyamine; N-oxide; hydrazine; hydrazide; hydrazone; azide; isocyanate; isothiocyanate; cyanate; thiocyanate; B(OH)₂, or O(alkyl)aminocarbonyl.

A “cycloalkyl” group is a saturated, partially saturated, or unsaturated cyclic alkyl group of from 3 to 10 carbon atoms having a single cyclic ring or multiple condensed or bridged rings which can be optionally substituted with from 1 to 3 alkyl groups. In some embodiments, the cycloalkyl group has 3 to 8 ring members, whereas in other embodiments the number of ring carbon atoms ranges from 3 to 5, 3 to 6, or 3 to 7. A cycloalkyl comprising more than one ring may be fused, spiro, or bridged, or combinations thereof. Such cycloalkyl groups include, by way of example, single ring structures such as cyclopropyl, cyclobutyl, cyclopentyl, cyclohexyl, cycloheptyl, cyclooctyl, 1-methylcyclopropyl, 2-methylcyclopentyl, 2-methylcyclooctyl, and the like, or multiple or bridged ring structures such as 1-bicyclo[1.1.1]pentyl, bicyclo[2.1.1]hexyl, bicyclo[2.2.1]heptyl, bicyclo[2.2.2]octyl, adamantyl and the like. Examples of unsaturared cycloalkyl groups include cyclohexenyl, cyclopentenyl, cyclohexadienyl, butadienyl, pentadienyl, hexadienyl, among others. A cycloalkyl group can be substituted or unsubstituted. Such substituted cycloalkyl groups include, by way of example, cyclohexanol and the like.

An “aryl” group is an aromatic carbocyclic group of from 6 to 14 carbon atoms having a single ring (e.g., phenyl) or multiple condensed rings (e.g., naphthyl or anthryl). In some embodiments, aryl groups contain 6-14 carbons, and in others from 6 to 12 or even 6 to 10 carbon atoms in the ring portions of the groups. Particular aryls include phenyl, biphenyl, naphthyl and the like. An aryl group can be substituted or unsubstituted. The phrase “aryl groups” also includes groups containing fused rings, such as fused aromatic-aliphatic ring systems (e.g., indanyl, tetrahydronaphthyl, and the like).

A “heterocyclyl” is an aromatic (also referred to as heteroaryl) or non-aromatic cycloalkyl in which one to four of the ring carbon atoms are independently replaced with a heteroatom from the group consisting of O, S and N. In some embodiments, heterocyclyl groups include 3 to 10 ring members, whereas other such groups have 3 to 5, 3 to 6, or 3 to 8 ring members. Heterocyclyls can also be bonded to other groups at any ring atom (i.e., at any carbon atom or heteroatom of the heterocyclic ring). A heterocyclyl group can be substituted or unsubstituted. A heterocyclyl group may include multiple condensed rings including, but are not limited to, bicyclic, tricyclic, and quadracylic rings, as well as bridged or spirocyclic ring systems. Heterocyclyl groups encompass unsaturated, partially saturated and saturated ring systems, such as, for example, imidazolyl, imidazolinyl and imidazolidinyl (e.g., imidazolidin-4-one or imidazolidin-2,4-dionyl) groups. The phrase heterocyclyl includes fused ring species, including those comprising fused aromatic and non-aromatic groups, such as, for example, 1- and 2-aminotetraline, benzotriazolyl (e.g., 1H-benzo[d][1,2,3]triazolyl), benzimidazolyl (e.g., 1H-benzo[d]imidazolyl), 2,3-dihydrobenzo[1,4]dioxinyl, and benzo[1,3]dioxolyl. The phrase also includes bridged polycyclic ring systems containing a heteroatom such as, but not limited to, quinuclidyl. Representative examples of a heterocyclyl group include, but are not limited to, aziridinyl, azetidinyl, azepanyl, oxetanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, dioxolyl, furanyl, thiophenyl, pyrrolyl, pyrrolinyl, imidazolyl, imidazolinyl, pyrazolyl, pyrazolinyl, triazolyl, tetrazolyl, oxazolyl, isoxazolyl, benzisoxazolyl (e.g., benzo[d]isoxazolyl), thiazolyl, thiazolinyl, isothiazolyl, thiadiazolyl, oxadiazolyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dioxyl, dithianyl, pyranyl, pyridyl, pyrimidyl, pyridazinyl, pyrazinyl, triazinyl, dihydropyridyl, dihydrodithiinyl, dihydrodithionyl, 1,4-dioxaspiro[4.5]decanyl, homopiperazinyl, quinuclidyl, indolyl (e.g., indolyl-2-onyl or isoindolin-1-onyl), indolinyl, isoindolyl, isoindolinyl, azaindolyl (pyrrolopyridyl or 1H-pyrrolo[2,3-b]pyridyl), indazolyl, indolizinyl, benzotriazolyl (e.g., 1H-benzo[d][1,2,3]triazolyl), benzimidazolyl (e.g., 1H-benzo[d]imidazolyl or 1H-benzo[d]imidazol-2(3H)-onyl), benzofuranyl, benzothiophenyl, benzothiazolyl, benzoxadiazolyl, benzoxazinyl, benzodithiinyl, benzoxathiinyl, benzothiazinyl, benzoxazolyl (i.e., benzo[d]oxazolyl), benzothiazolyl, benzothiadiazolyl, benzo[1,3]dioxolyl, pyrazolopyridyl (for example, 1H-pyrazolo[3,4-b]pyridyl, 1H-pyrazolo[4,3-b]pyridyl), imidazopyridyl (e.g., azabenzimidazolyl or 1H-imidazo[4,5-b]pyridyl), triazolopyridyl, isoxazolopyridyl, purinyl, xanthinyl, adeninyl, guaninyl, quinolinyl, isoquinolinyl (e.g., 3,4-dihydroisoquinolin-1(2H)-onyl), quinolizinyl, quinoxalinyl, quinazolinyl, cinnolinyl, phthalazinyl, naphthyridinyl, pteridinyl, thianaphthalenyl, dihydrobenzothiazinyl, dihydrobenzofuranyl, dihydroindolyl, dihydrobenzodioxinyl, tetrahydroindolyl, tetrahydroindazolyl, tetrahydrobenzimidazolyl, tetrahydrobenzotriazolyl, tetrahydropyrrolopyridyl, tetrahydropyrazolopyridyl, tetrahydroimidazopyridyl, tetrahydrotriazolopyridyl, tetrahydropyrimidin-2(1H)-one and tetrahydroquinolinyl groups. Representative non-aromatic heterocyclyl groups do not include fused ring species that comprise a fused aromatic group. Examples of non-aromatic heterocyclyl groups include aziridinyl, azetidinyl, azepanyl, pyrrolidyl, imidazolidinyl (e.g., imidazolidin-4-onyl or imidazolidin-2,4-dionyl), pyrazolidinyl, thiazolidinyl, tetrahydrothiophenyl, tetrahydrofuranyl, piperidyl, piperazinyl (e.g., piperazin-2-onyl), morpholinyl, thiomorpholinyl, tetrahydropyranyl (e.g., tetrahydro-2H-pyranyl), tetrahydrothiopyranyl, oxathianyl, dithianyl, 1,4-dioxaspiro[4.5]decanyl, homopiperazinyl, quinuclidyl, or tetrahydropyrimidin-2(1H)-one. Representative substituted heterocyclyl groups may be mono-substituted or substituted more than once, such as, but not limited to, pyridyl or morpholinyl groups, which are 2-, 3-, 4-, 5-, or 6-substituted, or disubstituted with various substituents such as those listed below.

A “heteroaryl” group is an aryl ring system having one to four heteroatoms as ring atoms in a heteroaromatic ring system, wherein the remainder of the atoms are carbon atoms. In some embodiments, heteroaryl groups contain 3 to 6 ring atoms, and in others from 6 to 9 or even 6 to 10 atoms in the ring portions of the groups. Suitable heteroatoms include oxygen, sulfur and nitrogen. In certain embodiments, the heteroaryl ring system is monocyclic or bicyclic. Non-limiting examples include but are not limited to, groups such as pyrrolyl, pyrazolyl, imidazolyl, triazolyl, tetrazolyl, oxazolyl, isoxazolyl, benzisoxazolyl (e.g., benzo[d]isoxazolyl), thiazolyl, pyrolyl, pyridazinyl, pyrimidyl, pyrazinyl, thiophenyl, benzothiophenyl, furanyl, benzofuranyl, indolyl (e.g., indolyl-2-onyl or isoindolin-1-onyl), azaindolyl (pyrrolopyridyl or 1H-pyrrolo[2,3-b]pyridyl), indazolyl, benzimidazolyl (e.g., 1H-benzo[d]imidazolyl), imidazopyridyl (e.g., azabenzimidazolyl or 1H-imidazo[4,5-b]pyridyl), pyrazolopyridyl, triazolopyridyl, benzotriazolyl (e.g., 1H-benzo[d][1,2,3]triazolyl), benzoxazolyl (e.g., benzo[d]oxazolyl), benzothiazolyl, benzothiadiazolyl, isoxazolopyridyl, thianaphthalenyl, purinyl, xanthinyl, adeninyl, guaninyl, quinolinyl, isoquinolinyl (e.g., 3,4-dihydroisoquinolin-1(2H)-onyl), tetrahydroquinolinyl, quinoxalinyl, and quinazolinyl groups.

As used herein, “spirocyclic ring” refers to two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings.

A “cycloalkylalkyl” group is a radical of the formula: -alkyl-cycloalkyl, wherein alkyl and cycloalkyl are as defined above. Substituted cycloalkylalkyl groups may be substituted at the alkyl, the cycloalkyl, or both the alkyl and the cycloalkyl portions of the group. Representative cycloalkylalkyl groups include but are not limited to methylcyclopropyl, methylcyclobutyl, methylcyclopentyl, methylcyclohexyl, ethylcyclopropyl, ethylcyclobutyl, ethylcyclopentyl, ethylcyclohexyl, propylcyclopentyl, propylcyclohexyl and the like.

An “aralkyl” group is a radical of the formula: -alkyl-aryl, wherein alkyl and aryl are defined above. Substituted aralkyl groups may be substituted at the alkyl, the aryl, or both the alkyl and the aryl portions of the group. Representative aralkyl groups include but are not limited to benzyl and phenethyl groups and fused (cycloalkylaryl)alkyl groups such as 4-ethyl-indanyl.

An “heterocyclylalkyl” group is a radical of the formula: -alkyl-heterocyclyl, wherein alkyl and heterocyclyl are defined above. Substituted heterocyclylalkyl groups may be substituted at the alkyl, the heterocyclyl, or both the alkyl and the heterocyclyl portions of the group. Representative heterocylylalkyl groups include but are not limited to 4-ethyl-morpholinyl, 4-propylmorpholinyl, furan-2-yl methyl, furan-3-yl methyl, pyridin-3-yl methyl, tetrahydrofuran-2-yl ethyl, and indol-2-yl propyl.

A “halogen” is fluorine, chlorine, bromine or iodine.

A “hydroxyalkyl” group is an alkyl group as described above substituted with one or more hydroxy groups.

An “alkoxy” or “alkoxyl” group is —O-(alkyl), wherein alkyl is defined above.

An “alkoxyalkyl” group is -(alkyl)-O-(alkyl), wherein alkyl is defined above.

An “amino” group is a radical of the formula: —NH₂.

An “alkylamino” group is a radical of the formula: —NH-alkyl or —N(alkyl)₂, wherein each alkyl is independently as defined above.

A “carboxy” group is a radical of the formula: —C(O)OH.

An “aminocarbonyl” group is a radical of the formula: —C(O)N(R^{#})₂, —C(O)NH(R^{#}) or —C(O)NH₂, wherein each R^{#} is independently a substituted or unsubstituted alkyl, cycloalkyl, aryl, aralkyl, heterocyclyl or heterocyclyl group as defined herein.

An “acylamino” group is a radical of the formula: —NHC(O)(R^{#}) or —N(alkyl)C(O)(R^{#}), wherein each alkyl and R^{#} are independently as defined above.

A “sulfonylamino” group is a radical of the formula: —NHSO₂(R^{#}) or —N(alkyl)SO₂(R^{#}), wherein each alkyl and R^{#} are defined above.

A “urea” group is a radical of the formula: —N(alkyl)C(O)N(R^{#})₂, —N(alkyl)C(O)NH(R^{#}), —N(alkyl)C(O)NH₂, —NHC(O)N(R^{#})₂, —NHC(O)NH(R^{#}), or —NH(CO)NHR^{#}, wherein each alkyl and R^{#} are independently as defined above.

When the groups described herein, with the exception of alkyl group, are said to be “substituted,” they may be substituted with any appropriate substituent or substituents. Illustrative examples of substituents are those found in the exemplary compounds and embodiments disclosed herein, as well as halogen (chloro, iodo, bromo, or fluoro); alkyl; hydroxyl; alkoxy; alkoxyalkyl; amino; alkylamino; carboxy; nitro; cyano; thiol; thioether; imine; imide; amidine; guanidine; enamine; aminocarbonyl; acylamino; phosphonato; phosphine; thiocarbonyl; sulfonyl; sulfone; sulfonamide; ketone; aldehyde; ester; urea; urethane; oxime; hydroxyl amino; alkoxyamine; aralkoxyamine; N-oxide; hydrazine; hydrazide; hydrazone; azide; isocyanate; isothiocyanate; cyanate; thiocyanate; oxo (═O); B(OH)₂, O(alkyl)aminocarbonyl; cycloalkyl, which may be monocyclic or fused or non-fused polycyclic (e.g., cyclopropyl, cyclobutyl, cyclopentyl, or cyclohexyl), or a heterocyclyl, which may be monocyclic or fused or non-fused polycyclic (e.g., pyrrolidyl, piperidyl, piperazinyl, morpholinyl, or thiazinyl); monocyclic or fused or non-fused polycyclic aryl or heteroaryl (e.g., phenyl, naphthyl, pyrrolyl, indolyl, furanyl, thiophenyl, imidazolyl, oxazolyl, isoxazolyl, thiazolyl, triazolyl, tetrazolyl, pyrazolyl, pyridyl, quinolinyl, isoquinolinyl, acridinyl, pyrazinyl, pyridazinyl, pyrimidyl, benzimidazolyl, benzothiophenyl, or benzofuranyl) aryloxy; aralkyloxy; heterocyclyloxy; and heterocyclyl alkoxy.

As used herein, the term “pharmaceutically acceptable salt(s)” refers to a salt prepared from a pharmaceutically acceptable non-toxic acid or base including an inorganic acid and base and an organic acid and base. Suitable pharmaceutically acceptable base addition salts of the compounds of formula (I) include, but are not limited to those well-known in the art, see for example, Remington's Pharmaceutical Sciences, 18^{th} eds., Mack Publishing, Easton PA (1990) or Remington: The Science and Practice of Pharmacy, 19^{th} eds., Mack Publishing, Easton PA (1995).

As used herein and unless otherwise indicated, the term “stereoisomer” or “stereomerically pure” means one stereoisomer of a compound that is substantially free of other stereoisomers of that compound. For example, a stereomerically pure compound having one chiral center will be substantially free of the opposite enantiomer of the compound. A stereomerically pure compound having two chiral centers will be substantially free of other diastereomers of the compound. A typical stereomerically pure compound comprises greater than about 80% by weight of one stereoisomer of the compound and less than about 20% by weight of other stereoisomers of the compound, greater than about 90% by weight of one stereoisomer of the compound and less than about 10% by weight of the other stereoisomers of the compound, greater than about 95% by weight of one stereoisomer of the compound and less than about 5% by weight of the other stereoisomers of the compound, or greater than about 97% by weight of one stereoisomer of the compound and less than about 3% by weight of the other stereoisomers of the compound. The compounds can have chiral centers and can occur as racemates, individual enantiomers or diastereomers, and mixtures thereof. All such isomeric forms are included within the embodiments disclosed herein, including mixtures thereof.

The use of stereomerically pure forms of such compounds, as well as the use of mixtures of those forms, are encompassed by the embodiments disclosed herein. For example, mixtures comprising equal or unequal amounts of the enantiomers of a particular compound may be used in methods and compositions disclosed herein. These isomers may be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, IN, 1972).

It should also be noted the compounds can include E and Z isomers, or a mixture thereof, and cis and trans isomers or a mixture thereof. In certain embodiments, the compounds are isolated as either the E or Z isomer. In other embodiments, the compounds are a mixture of the E and Z isomers.

“Tautomers” refers to isomeric forms of a compound that are in equilibrium with each other. The concentrations of the isomeric forms will depend on the environment the compound is found in and may be different depending upon, for example, whether the compound is a solid or is in an organic or aqueous solution. For example, in aqueous solution, pyrazoles may exhibit the following isomeric forms, which are referred to as tautomers of each other:

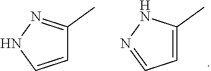

As readily understood by one skilled in the art, a wide variety of functional groups and other stuctures may exhibit tautomerism and all tautomers of compounds of formula (I) are within the scope of the present invention.

It should also be noted the compounds can contain unnatural proportions of atomic isotopes at one or more of the atoms. For example, the compounds may be radiolabeled with radioactive isotopes, such as for example tritium (³H), iodine-125 (¹²⁵I), sulfur-35 (³⁵S), or carbon-14 (¹⁴C), or may be isotopically enriched, such as with deuterium (²H), carbon-13 (¹³C), or nitrogen-15 (¹⁵N). As used herein, an “isotopologue” is an isotopically enriched compound. The term “isotopically enriched” refers to an atom having an isotopic composition other than the natural isotopic composition of that atom. “Isotopically enriched” may also refer to a compound containing at least one atom having an isotopic composition other than the natural isotopic composition of that atom. The term “isotopic composition” refers to the amount of each isotope present for a given atom. Radiolabeled and isotopically enriched compounds are useful as therapeutic agents, e.g., cancer and inflammation therapeutic agents, research reagents, e.g., binding assay reagents, and diagnostic agents, e.g., in vivo imaging agents. All isotopic variations of the compounds as described herein, whether radioactive or not, are intended to be encompassed within the scope of the embodiments provided herein. In some embodiments, there are provided isotopologues of the compounds, for example, the isotopologues are deuterium, carbon-13, or nitrogen-15 enriched compounds.

“Treating” as used herein, means an alleviation, in whole or in part, of a disorder, disease or condition, or one or more of the symptoms associated with a disorder, disease, or condition, or slowing or halting of further progression or worsening of those symptoms, or alleviating or eradicating the cause(s) of the disorder, disease, or condition itself. In some embodiments, “treating” means an alleviation, in whole or in part, of a disorder, disease or condition, or a slowing, or halting of further progression or worsening of those symptoms. In another embodiment, “treating” means and alleviation, in whole or in part, of a disorder, disease or condition, or symptoms associated with a condition, wherein the condition is treatable or preventable by inhibition of Cbl-b.

“Preventing” as used herein, means a method of delaying and/or precluding the onset, recurrence or spread, in whole or in part, of a disorder, disease or condition; barring a subject from acquiring a disorder, disease, or condition; or reducing a subject's risk of acquiring a disorder, disease, or condition. In one embodiment, the condition is a condition, treatable or preventable by inhibition of Cbl-b.

The term “effective amount” in connection with a compound means an amount capable of treating or preventing a disorder, disease or condition, or symptoms thereof, disclosed herein.

The term “subject” includes an animal, including, but not limited to, an animal such a cow, monkey, horse, sheep, pig, chicken, turkey, quail, cat, dog, mouse, rat, rabbit or guinea pig, in one embodiment a mammal, in another embodiment a human.

### Compounds

Aspect 1: Provided herein are compounds having the following formula (I):

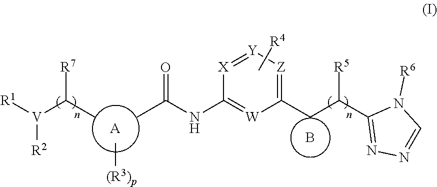

and pharmaceutically acceptable salts, tautomers, stereoisomers, enantiomers, isotopologues, and prodrugs thereof,
wherein:
each of V, W, X, Y, and Z is, independently CH or N;
each of R¹ and R² is, independently, hydrogen, substituted or unsubstituted C₁₋₈ alkyl, substituted or unsubstituted cycloalkyl, substituted or unsubstituted non-aromatic heterocyclyl, substituted or unsubstituted saturated cycloalkylalkyl, substituted or unsubstituted non-aromatic heterocyclylalkyl, or R¹ and R² together with the atom which R¹ and R² connect to form a substituted or unsubstituted cycloalkyl or non-aromatic heterocyclyl;
R³ is hydrogen, halogen, —CN, hydroxyl, substituted or unsubstituted C₁₋₈ alkyl, or substituted or unsubstituted saturated cycloalkyl;
R⁴ is hydrogen, halogen, substituted or unsubstituted C₁₋₈ alkyl, or substituted or unsubstituted saturated cycloalkyl;
R⁵ is hydrogen, halogen, substituted or unsubstituted C₁₋₈ alkyl, or substituted or unsubstituted saturated cycloalkyl;
R⁶ is hydrogen, or substituted or unsubstituted C₁₋₈ alkyl;
R⁷ is hydrogen, deuterium, halogen, or substituted or unsubstituted C₁₋₈ alkyl or substituted or unsubstituted saturated cycloalkyl;
ring A is substituted or unsubstituted aryl, or substituted or unsubstituted heteroaryl;
moiety B is substituted or unsubstituted aryl, substituted or unsubstituted cycloalkyl, substituted or unsubstituted cycloalkylalkyl, a substituted or unsubstituted saturated spiro bicyclic ring, or substituted or unsubstituted non-aromatic heterocyclyl; and
each of m, n and p is, independently, 0, 1, or 2.

In one embodiment, R⁷ is hydrogen, halogen, or substituted or unsubstituted C₁₋₈ alkyl or substituted or unsubstituted saturated cycloalkyl.

Aspect 2: In one embodiment, the compound having formula (I) is a compound of formula (IIa):

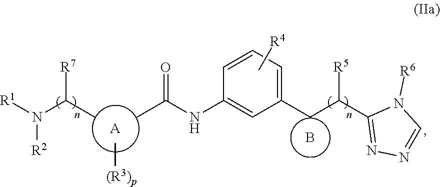

or a pharmaceutically acceptable salt, tautomer, stereoisomer, enantiomer, isotopologue, or prodrug thereof.

In one embodiment, the compound having formula (I) is a compound of formula (IIb):

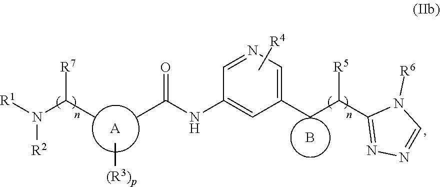

or a pharmaceutically acceptable salt, tautomer, stereoisomer, enantiomer, isotopologue, or prodrug thereof.

In one embodiment, R¹ is substituted or unsubstituted C₁₋₈ alkyl, substituted or unsubstituted cycloalkyl, or substituted or unsubstituted saturated cycloalkylalkyl.

In one embodiment, R¹ is 2,2,2-trifluoroethyl, cyclopropylmethyl, cyclobutyl, cyclopentyl, 3,3-difluorocyclopentyl, cyclohexyl, 3,3-difluoro-cyclohexyl, 3-trifluoromethylcyclohexyl, or 1-methylbicyclo[1.1.1]pentyl; preferably 2,2,2-trifluoroethyl, cyclopropylmethyl, cyclobutyl, cyclopentyl, 3,3-difluoro-cyclopentyl, cyclohexyl, 3,3-difluoro-cyclohexyl, 3-trifluoromethylcyclohexyl, or 1-methylbicyclo[1.1.1]pentyl.

In one embodiment, R¹ is substituted or unsubstituted C₂₋₈ alkyl, substituted or unsubstituted cyclopropyl, substituted or unsubstituted cyclobutyl, substituted or unsubstituted cyclopentyl, substituted or unsubstituted cyclohexyl, substituted or unsubstituted cyclopropylmethyl, substituted or unsubstituted cyclobutylmethyl, substituted or unsubstituted cyclopentylmethyl, substituted or unsubstituted cyclohexylmethyl;
more preferably 2-methylpropyl, neopentyl, (R)-(sec-butyl), 1-methylcyclopropyl, 1-methylcyclobutyl, (1S,3S)-3-methylcyclobutyl, 3-methylcyclobutyl, (R)-3,3-difluorocyclopentyl, cyclopropylmethyl, (S)-3,3-difluorocyclopentyl, (S)-1-cyclopropylethyl, (1-methylcyclopropyl)methyl, (1-fluorocyclopropyl)methyl, cyclobutylmethyl, 1-cyclobutylethyl, (S)-1-cyclobutylethyl, (1-methylcyclobutyl)methyl, (1-fluorocyclobutyl)methyl, cyclopentylmethyl, (1-methylcyclopentyl)methyl, (1-fluorocyclopentyl)methyl, 1-cyclopentylethyl, (S)-1-cyclopentylethyl, (1-methylcyclopentyl)methyl, cyclohexylmethyl, 1-cyclohexylethyl, (1-methylcyclohexyl)methyl, (4,4-difluorocyclohexyl)methyl, (3,3-difluorocyclohexyl)methyl, (3,3-difluorocyclopentyl)methyl, cyclohexyl, cycloheptyl.

In one embodiment, R² is hydrogen.

Aspect 3: In one embodiment, the compound having formula (I) is a compound of formula (IIIa):

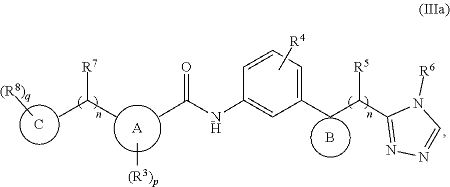

wherein moiety C is a substituted or unsubstituted saturated spiro bicyclic ring, substituted or unsubstituted non-aromatic heterocyclyl, or substituted or unsubstituted amino;
R⁸ is hydrogen, halogen, substituted or unsubstituted C₁₋₈ alkyl, substituted or unsubstituted C₁₋₈ alkoxyl, substituted or unsubstituted saturated cycloalkyl, or substituted or unsubstituted saturated cycloalkylalkyl; or two R⁸ together with the atom(s) which they connect to form a substituted or unsubstituted saturated cycloalkyl or substituted or unsubstituted saturated spiro cyclic ring; and
q is 0, 1, 2, or 3.

In one embodiment, the compound having formula (I) is a compound of formula (IIIb):

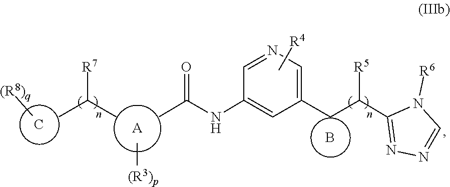

wherein moiety C is a substituted or unsubstituted saturated spiro bicyclic ring, substituted or unsubstituted non-aromatic heterocyclyl, or substituted or unsubstituted amino;
R⁸ is hydrogen, halogen, substituted or unsubstituted C₁₋₈ alkyl, substituted or unsubstituted C₁₋₈ alkoxyl, substituted or unsubstituted saturated cycloalkyl, or substituted or unsubstituted saturated cycloalkylalkyl; or two R⁸ together with the atom(s) which they connect to form a substituted or unsubstituted saturated cycloalkyl or substituted or unsubstituted saturated spiro cyclic ring; and
q is 0, 1, 2, or 3.

Aspect 4: In one embodiment, ring A is substituted or unsubstituted bicyclic heteroaryl.

Aspect 5: In one embodiment, ring A is

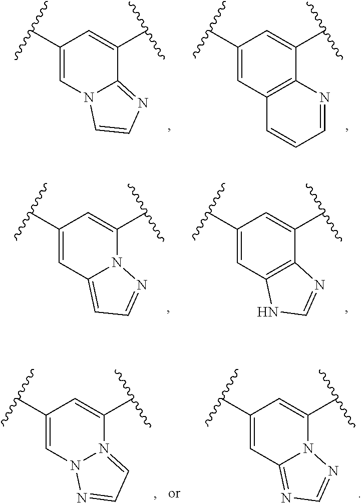

In one embodiment, wherein ring A is

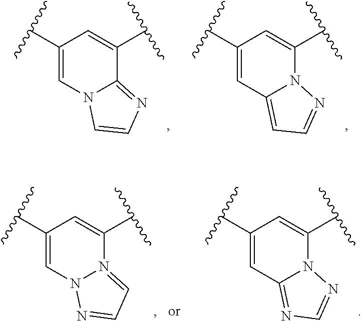

In one embodiment, R³ is hydrogen, halogen, or substituted or unsubstituted C₁₋₈ alkyl; preferably hydrogen, F, Cl, or methyl.

In one embodiment,

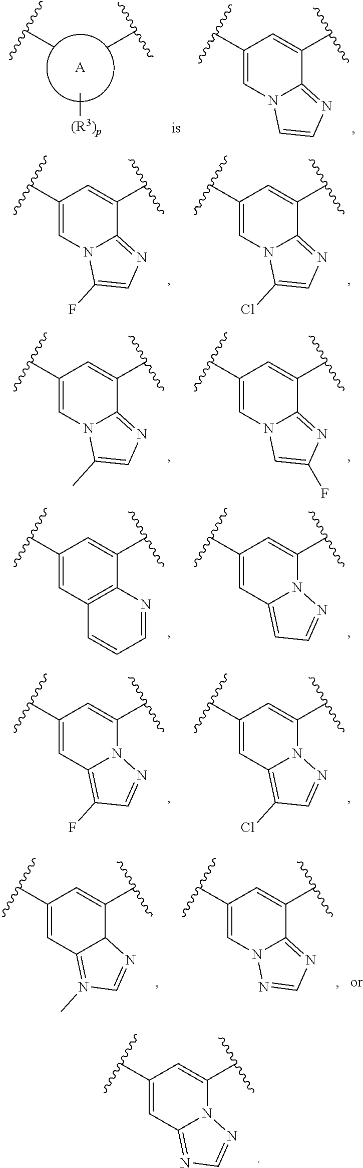

In one embodiment, moiety B is

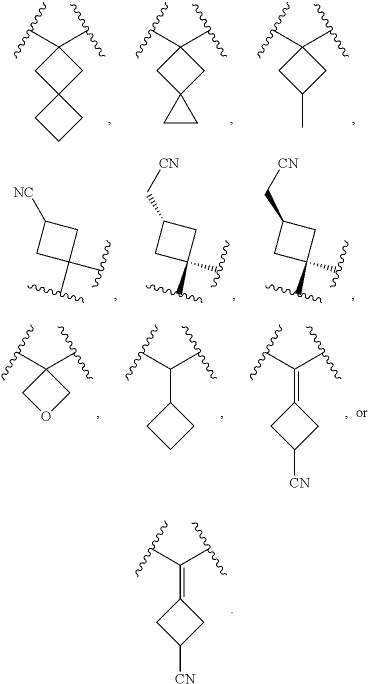

In one embodiment, moiety B is

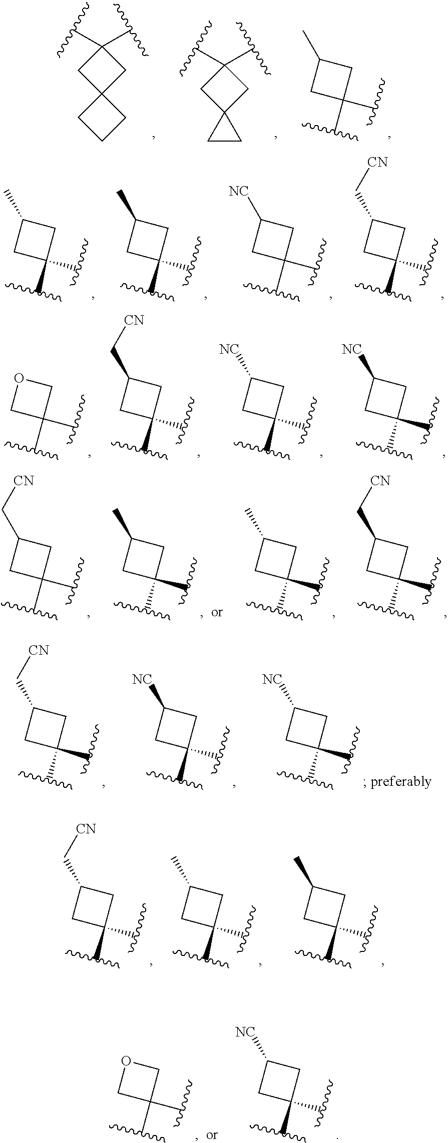

In one embodiment, moiety C is substituted or unsubstituted piperidyl, substituted or unsubstituted pyrrolidyl, substituted or unsubstituted azetidyl, substituted or unsubstituted azepanyl, or substituted or unsubstituted saturated spiro bicyclic ring, wherein one ring of the spiro bicyclic ring is substituted or unsubstituted piperidyl, substituted or unsubstituted pyrrolidyl, substituted or unsubstituted azetidyl, substituted or unsubstituted azepanyl and the other ring of the spiro bicyclic ring is cyclopropyl, cyclobutyl, cyclopentyl, or cyclohexyl.

In one embodiment, moiety C is substituted or unsubstituted aminoalkyl, substituted or unsubstituted cyclohexylmethylamino, substituted or unsubstituted cyclopentylmethylamino, substituted or unsubstituted cyclobutylmethylamino, or substituted or unsubstituted saturated cyclopropylmethylamino.

In one embodiment,

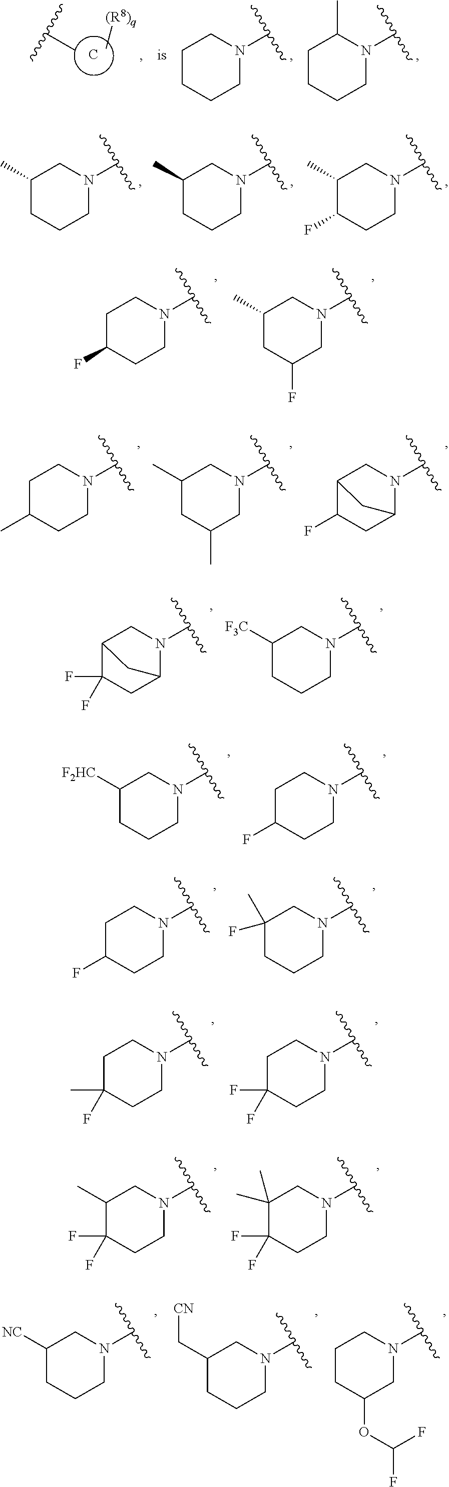

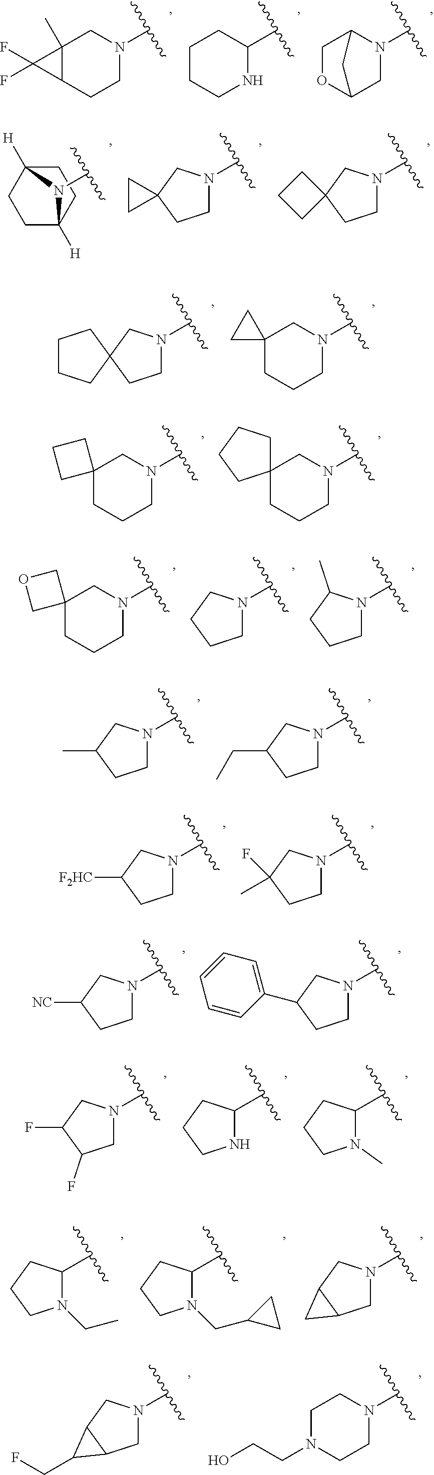

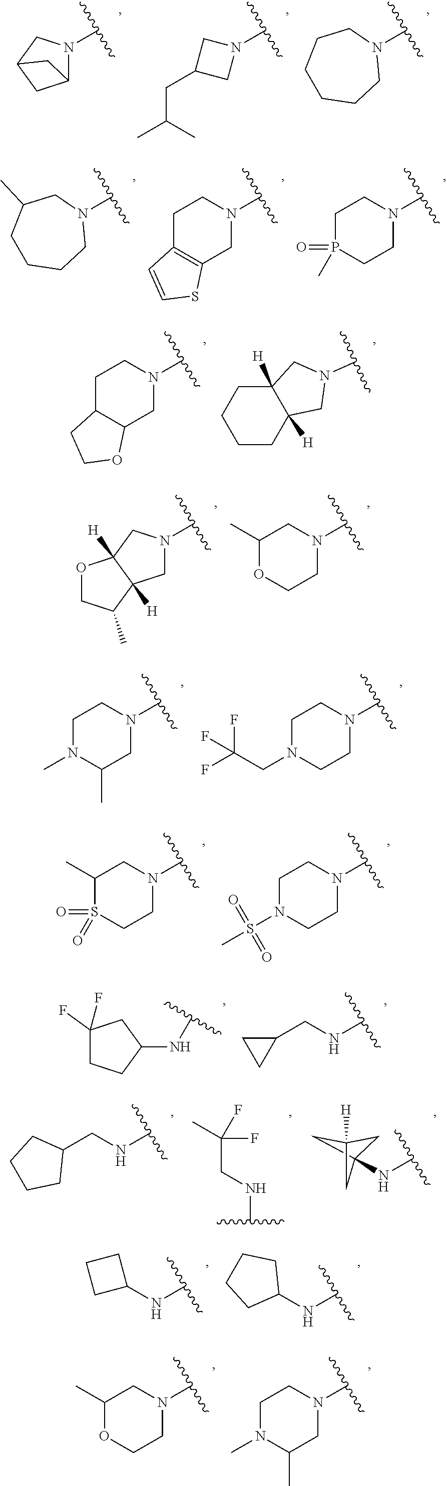

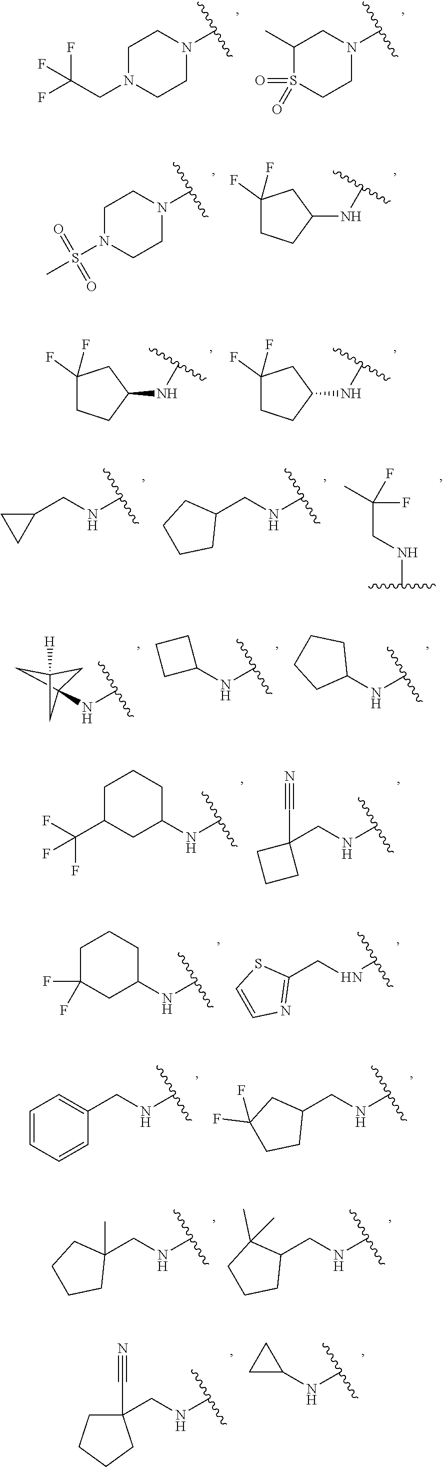

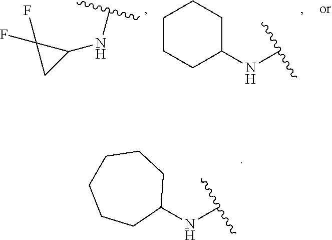 [SMILES: *NC1CC1(F)F ; *NC1CCCCC1 ; *NC1CCCCCC1]

In one embodiment,

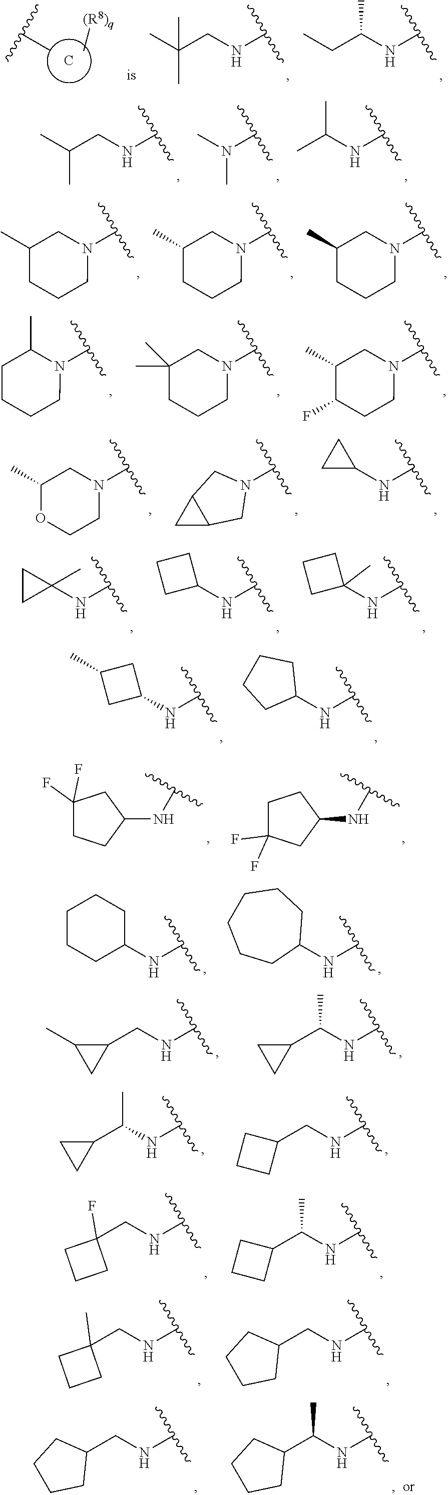

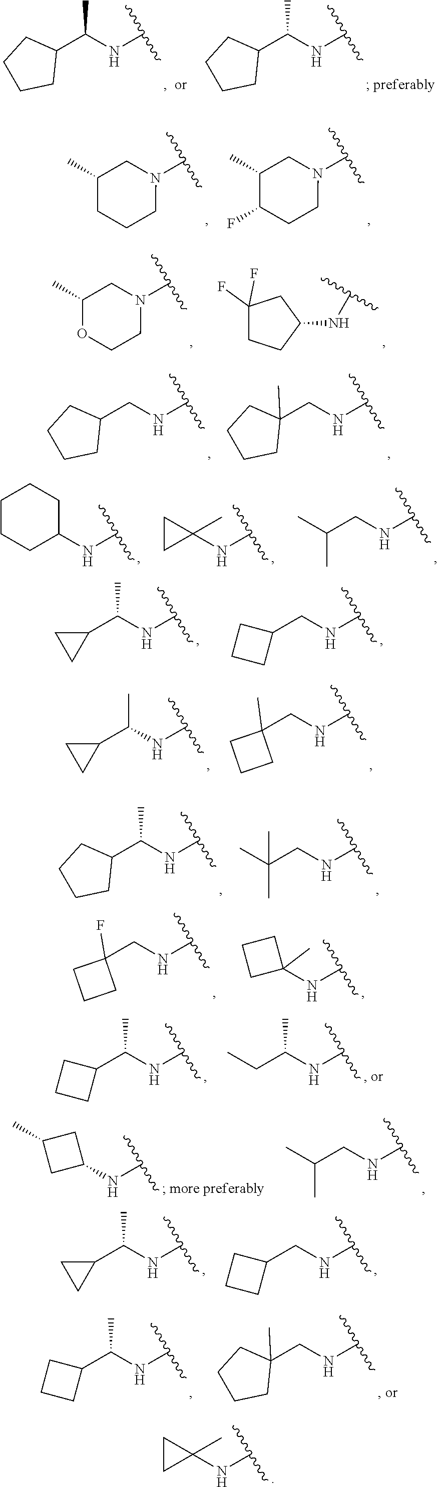

Aspect 6: In one embodiment, ring A is

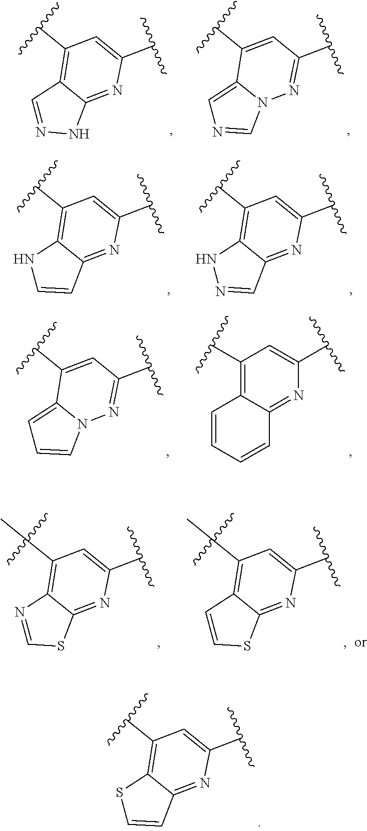

In one embodiment, ring A is

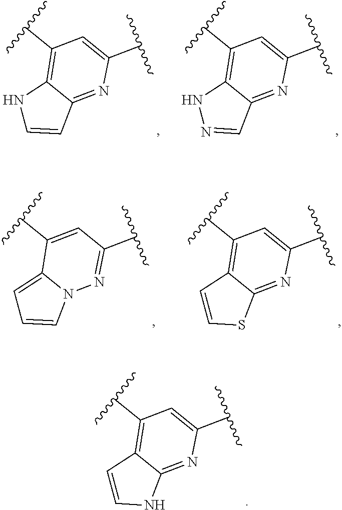 [SMILES: *c1cc(*)c2[nH]ccc2n1 ; *c1cc(*)c2[nH]ncc2n1 ; *c1cc(*)c2cccn2n1 ; *c1cc(*)c2ccsc2n1 ; *c1cc(*)c2cc[nH]c2n1]

In one embodiment, R³ is hydrogen, halogen, —CN, —CF₃, substituted or unsubstituted C₁₋₄ alkyl, or substituted or unsubstituted cycloalkyl.

In one embodiment, R³ is H, F, Cl, Br or methyl.

In one embodiment,

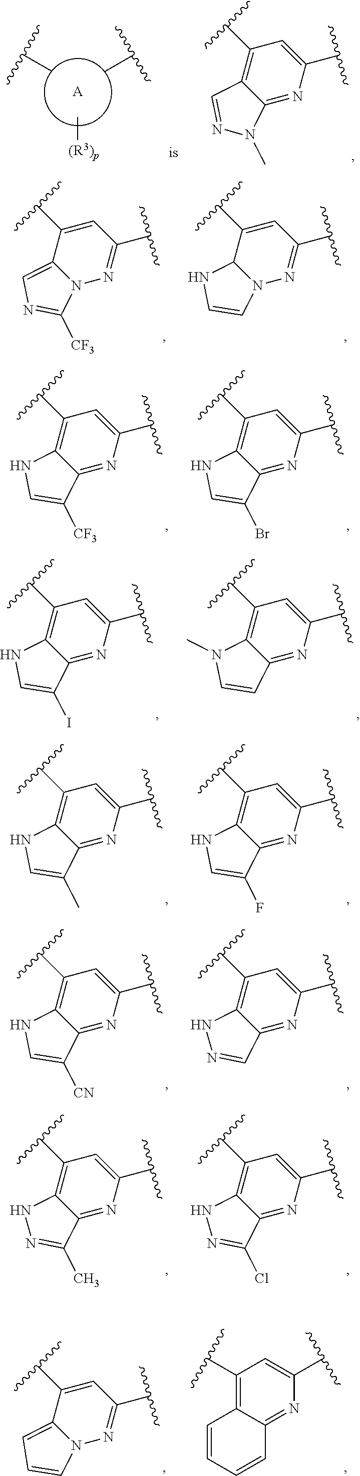

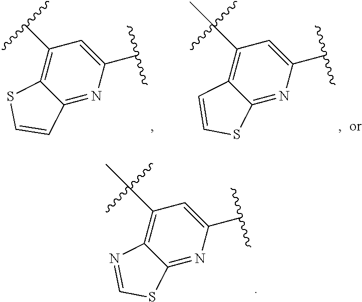 [SMILES: *c1cc(*)c2sccc2n1 ; *c1cc(*)c2ccsc2n1 ; *c1cc(*)c2ncsc2n1]

In one embodiment,

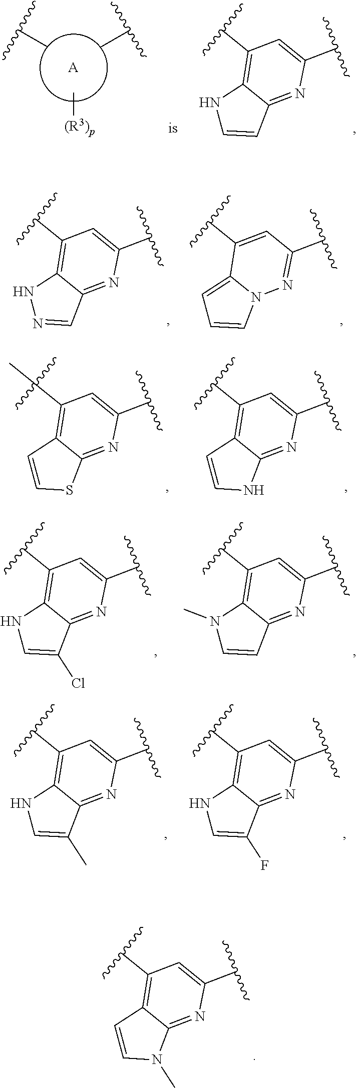

In one embodiment, moiety B is

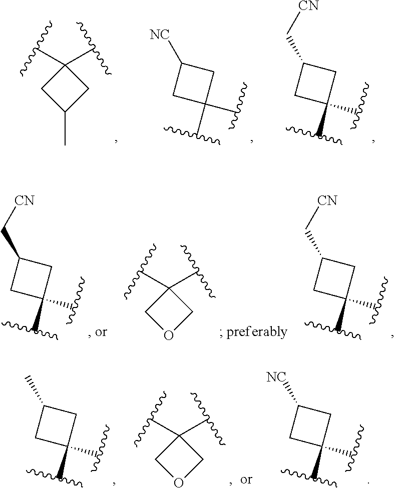

In one embodiment, moiety B is

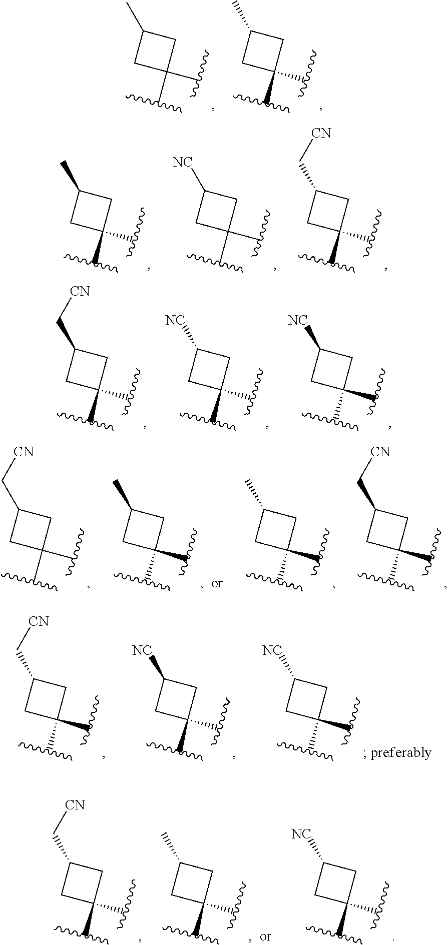

In one embodiment, moiety C is substituted or unsubstituted piperidyl, substituted or unsubstituted pyrrolidyl, substituted or unsubstituted azetidyl, or substituted or unsubstituted 5-azaspiro[2.4]heptyl.

In one embodiment, moiety C is substituted or unsubstituted alkylamino, substituted or unsubstituted cyclohexylmethylamino, substituted or unsubstituted cyclopentylmethylamino, substituted or unsubstituted cyclobutylmethylamino, or substituted or unsubstituted saturated cyclopropylmethylamino, substituted or unsubstituted azetidyl, substituted or unsubstituted azepanyl.

In one embodiment,

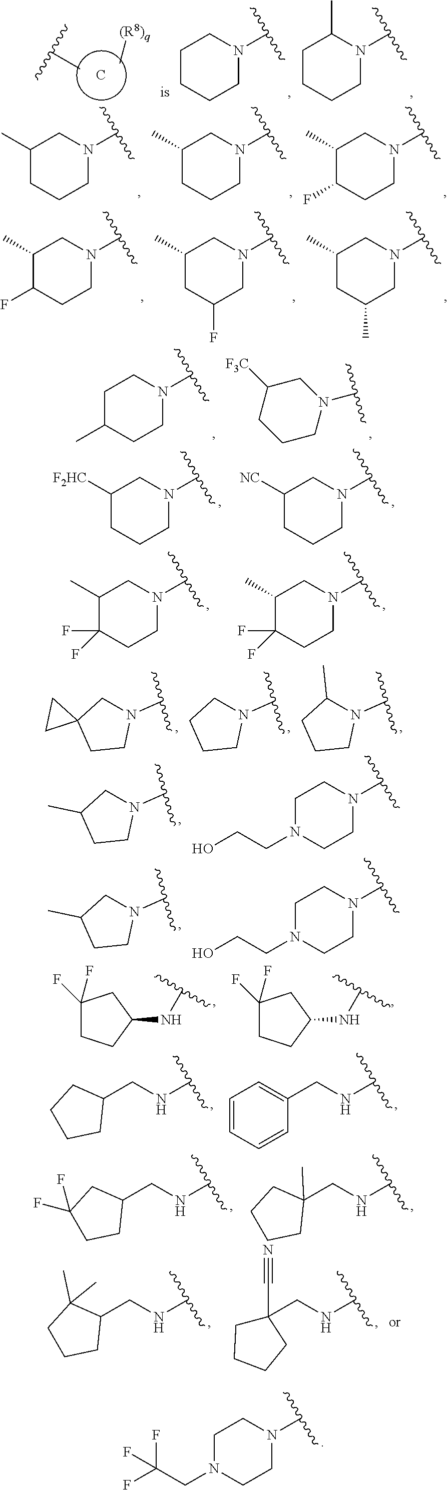

In one embodiment,

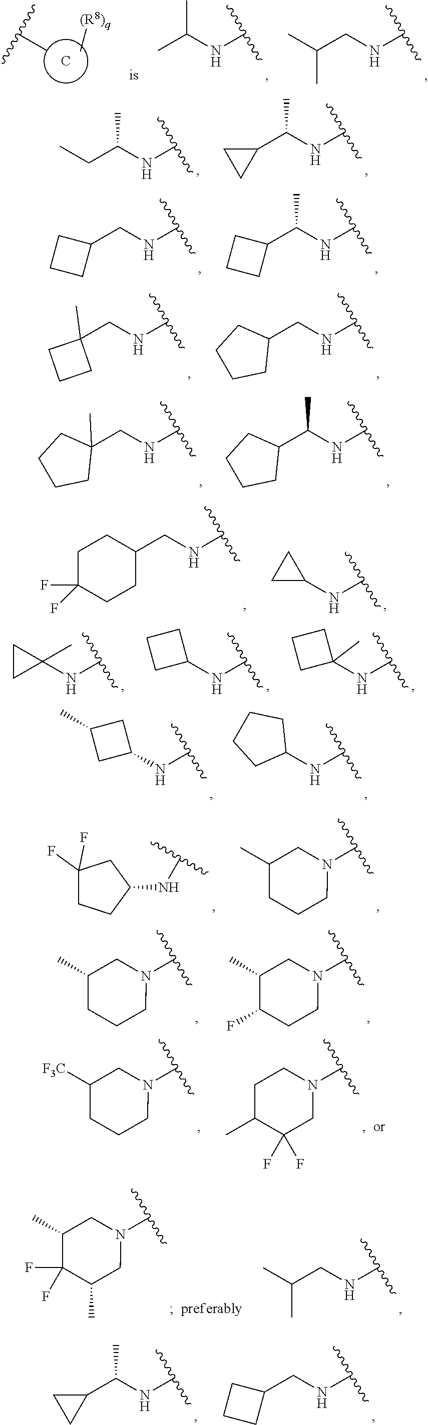

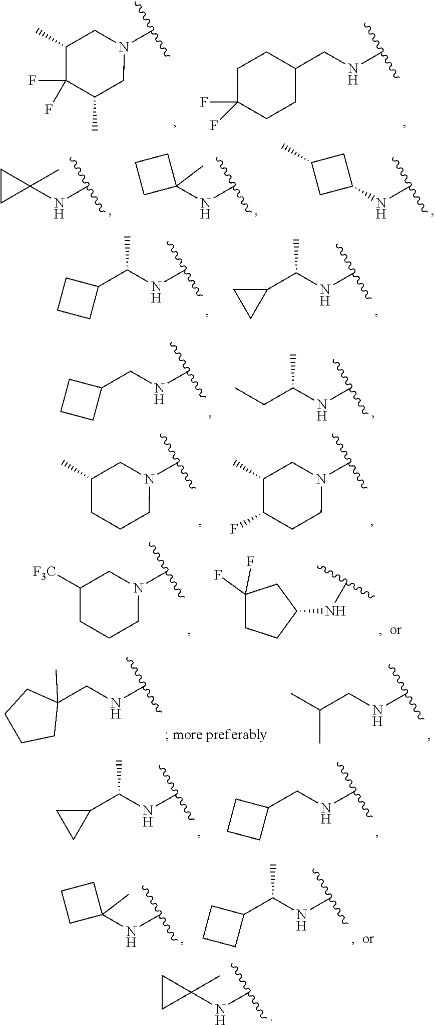

Aspect 7: In one embodiment, ring A is

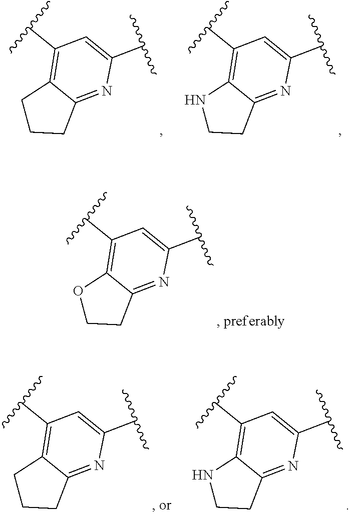 [SMILES: *c1cc(*)c2c(n1)CCC2 ; *c1cc(*)c2c(n1)CCN2 ; *c1cc(*)c2c(n1)CCO2 ; *c1cc(*)c2c(n1)CCC2 ; *c1cc(*)c2c(n1)CCN2]

In one embodiment, R³ is hydrogen, halogen, hydroxyl, or substituted or unsubstituted C₁₋₄ alkyl; preferably methyl, F, or spiro-cyclopropyl.

In one embodiment, p is 0, 1 or 2; or preferable 2.

In one embodiment,

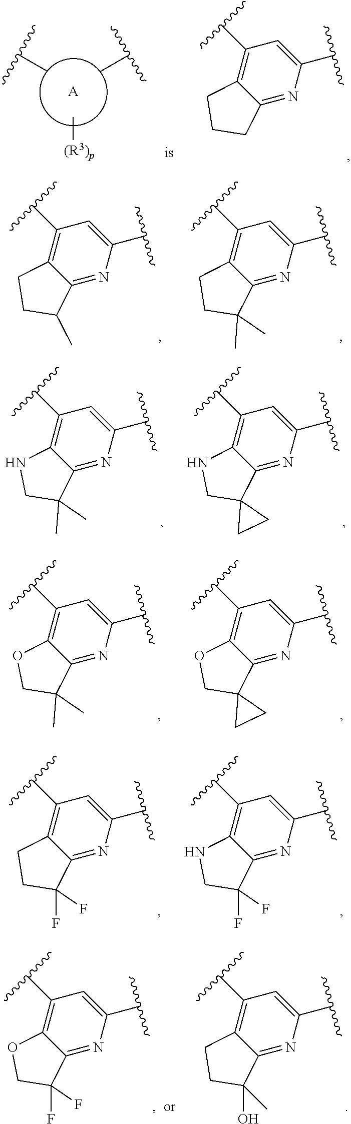

In one embodiment, moiety B is

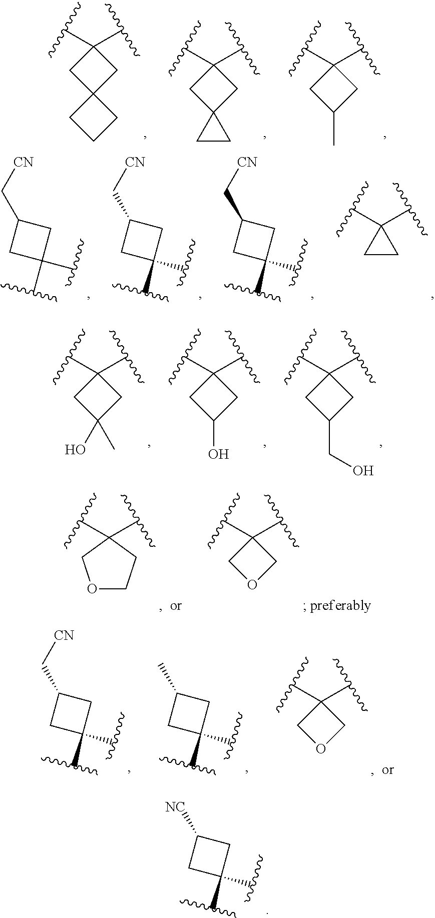

In one embodiment, moiety B is

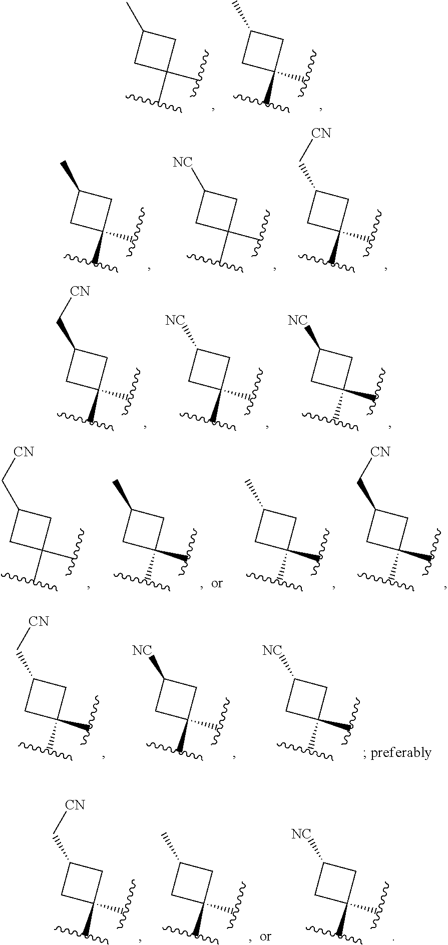

In one embodiment, moiety C is substituted or unsubstituted alkylamino, substituted or unsubstituted cyclohexylmethylamino, substituted or unsubstituted cyclopentylmethylamino, substituted or unsubstituted cyclobutylmethylamino, or substituted or unsubstituted saturated cyclopropylmethylamino, substituted or unsubstituted azetidyl, substituted or unsubstituted azepanyl, substituted or unsubstituted piperidyl, substituted or unsubstituted pyrrolidyl.

In one embodiment,

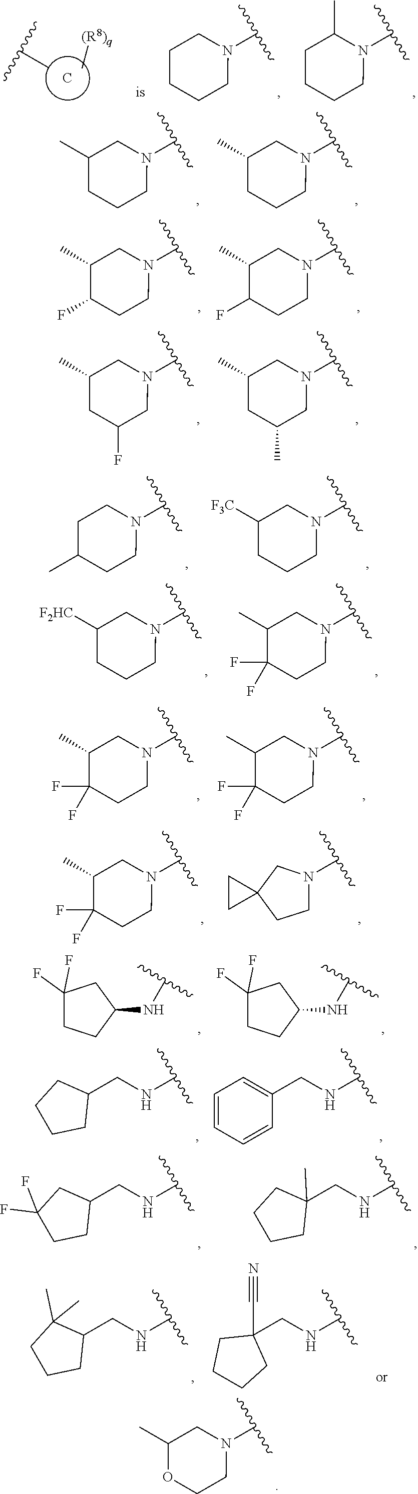

In one embodiment,

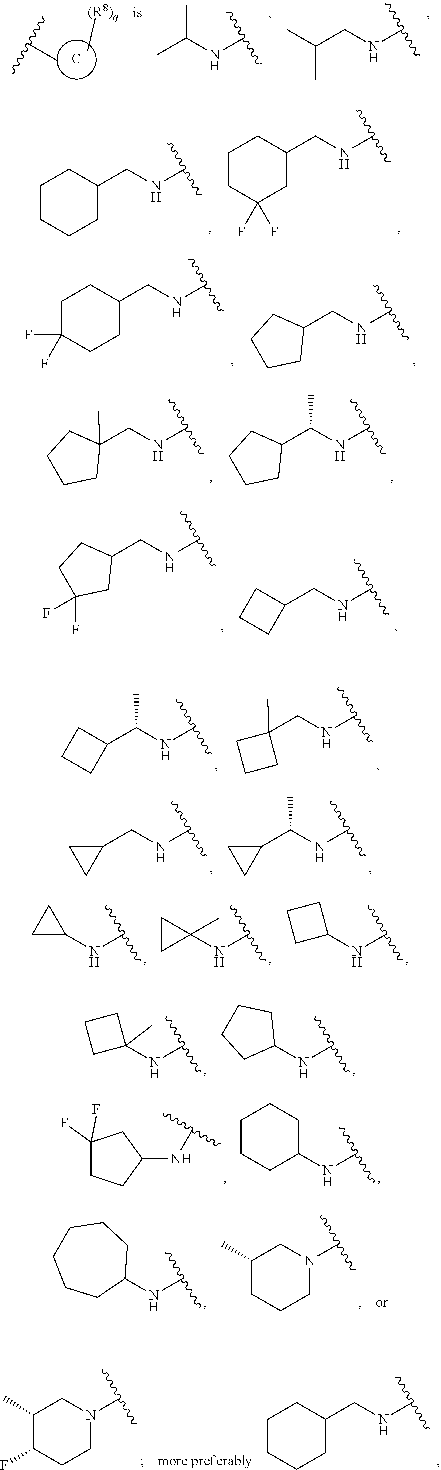

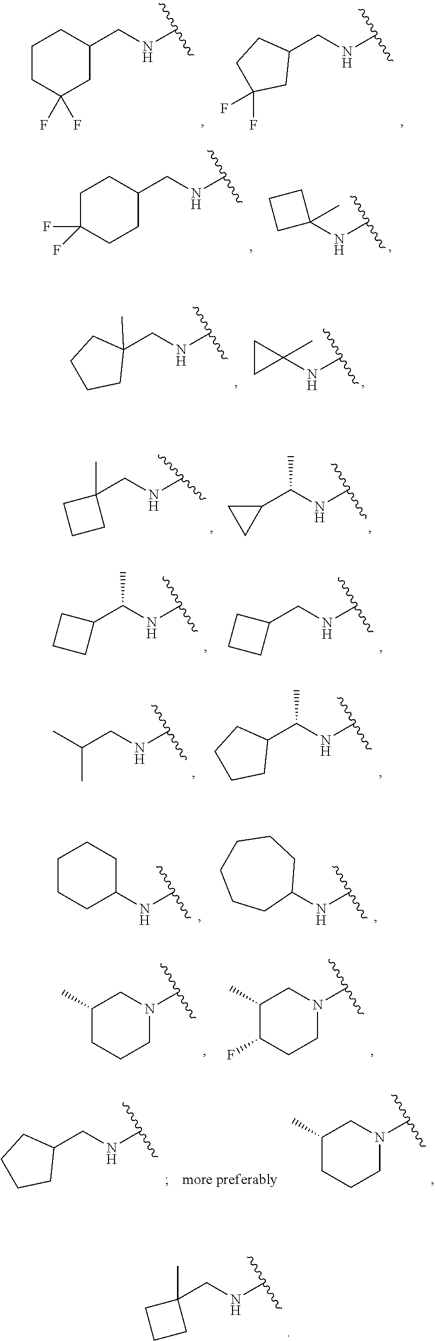

In one embodiment, R⁵ is hydrogen, substituted or unsubstituted C₁₋₈ alkyl, or substituted or unsubstituted saturated cycloalkyl; preferably hydrogen, or methyl.

In one embodiment, R⁶ is methyl or ethyl.

In one embodiment, R⁶ is methyl-d3.

In one embodiment, each of n and p is, independently, 0 or 1.

In one embodiment, m is 1.

In one embodiment, R⁷ is hydrogen, substituted or unsubstituted C₁₋₄ alkyl, or substituted or unsubstituted cycloalkyl.

In one embodiment, R⁷ is hydrogen, deuterium, methyl, ethyl, isopropyl or 2-hydroxyethyl; preferably R⁷ is hydrogen or methyl.

Aspect 8: In one embodiment, the compound is selected from Table 1, Table 2 and Table 3. In one embodiment, the compound having formula (I) is a compound selected from Table 1, Table 2 and Table 3.

Aspect 9: Provided here is a pharmaceutical composition comprising an effective amount of a compound provided herein, or a pharmaceutically acceptable salt, tautomer, isotopologue, stereoisomer, or prodrug thereof, and a pharmaceutically acceptable carrier, excipient or vehicle.

Aspect 10: Provided here is a method of modulating activity of an immune cell, comprising contacting said cell with an effective amount of a compound provided herein, or a pharmaceutically acceptable salt, tautomer, isotopologue, stereoisomer, or prodrug thereof. In one embodiment, the activity is the Cbl-b activity. In one embodiment, the immune cell is a T cell. In one embodiment, the immune cell is a B cell. In one embodiment, the immune cell is a NK cell.

Aspect 11: Provided here is a method for the treatment or prevention of a cancer responsive to Cbl-b activity, the methods comprising administering to a subject in need thereof an effective amount of a compound provided herein. In one embodiment, the cancer is hematologic cancer. In one embodiment, the hematologic cancer is is lymphoma, leukemia, myeloma, or glioblastoma.

Aspect 12: Provided herein is a kit for treating cancer, the kit comprising (a) a pharmaceutical composition comprising a compound provided herein; and (b) instructions for administration of an effective amount of the pharmaceutical composition comprising the Cbl-b inhibitor to treat cancer in an individual.

The present embodiments can be understood more fully by reference to the detailed description and examples, which are intended to exemplify non-limiting embodiments.

### Methods for Making Compounds

The Compounds can be made using conventional organic syntheses and commercially available starting materials. By way of example and not limitation, Compounds of formula (I) can be prepared as outlined in Schemes 1-12 shown below as well as in the examples set forth herein. It should be noted that one skilled in the art would know how to modify the procedures set forth in the illustrative schemes and examples to arrive at the desired products.

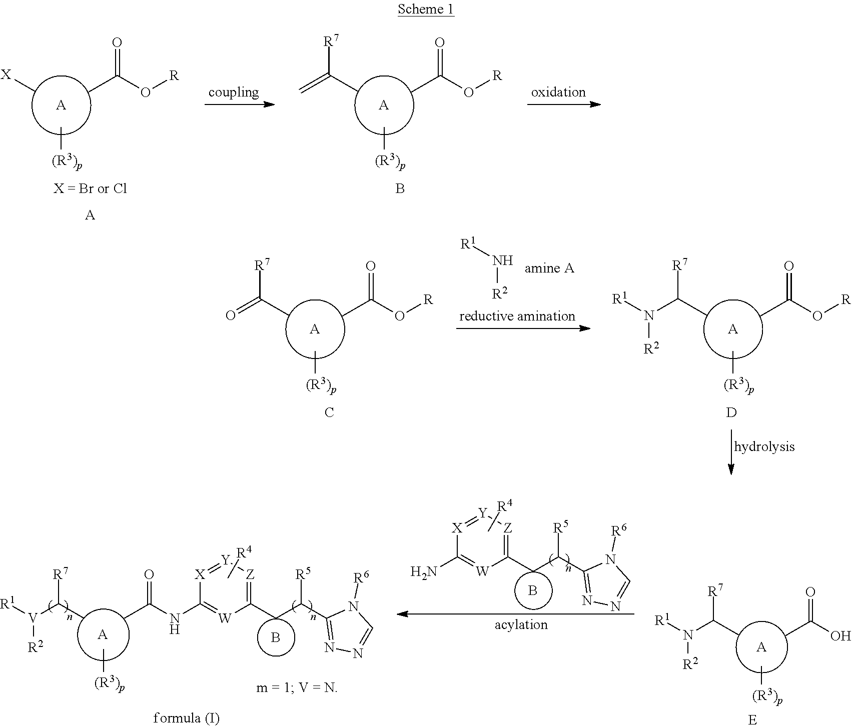

Scheme 1 outlines an approach to synthesizing the compounds defined as formula I (m is 1; V is nitrogen atom). Halogen substituted compound A is converted into compound B in Suzuki coupling conditions (e.g., K₂CO₃, Pd(dppf)Cl₂.); then compound B is oxidated to compound C under oxidation agent (e.g., K₂OsO₄, NaIO₄); compound C further undergoes reductive amination under reduction agent (e.g., STAB) with amine A to obtain compound D; compound E is prepared by hydrolysis compound D under basic condition (e.g., KOH, NaOH or LiOH) or acid condition (e.g., HCl); compound E is acylated with substituted aromatic amines under standard amide bond formation conditions (e.g., active ester formation (HATU or EDCI, DIPEA)) to yield the formula I (m is 1; V is nitrogen atom).

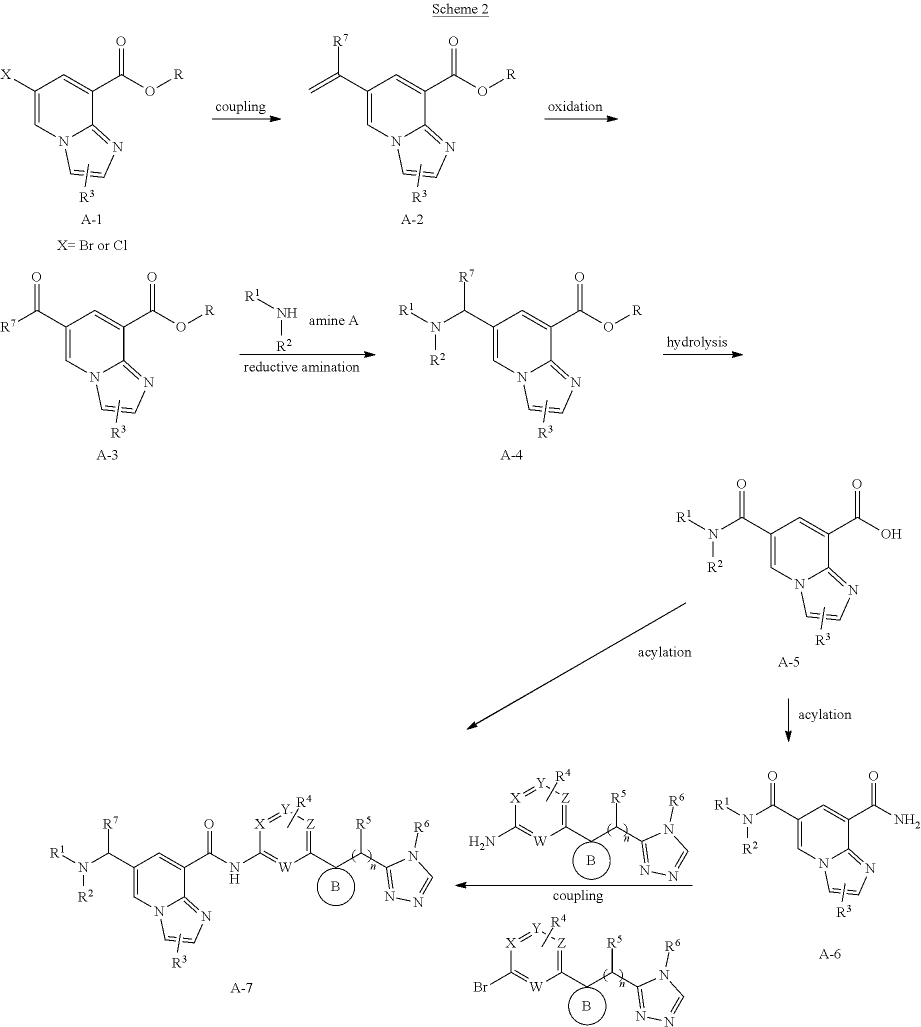

Scheme 2 illustrates an approach to synthesizing compound A-7. Halogen substituted compound A-1 is converted into compound A-2 in Suzuki coupling conditions (e.g., K₂CO₃, Pd(dppf)Cl₂.); then compound A-2 is oxidated to compound A-3 (e.g., K₂OsO₄, NaIO₄); compound A-3 further undergoes reductive amination under reduction agent (e.g., STAB) with amine A to obtain compound A-4; compound A-5 is prepared by hydrolysis of compound A-4 under basic condition (e.g., KOH, NaOH or LiOH) or acid condition (e.g., HCl); compound A-5 is acylated with substituted aromatic amines under standard amide bond formation conditions (e.g., active ester formation HATU or EDCI, DIPEA) to yield the compound A-7.

Compound A-5 is acylated to compound A-6 under standard amide bond formation conditions (e.g., active ester formation HATU or EDCI, DIPEA); then A-6 reacts with substituted aromatic halogen under standard carbon-nitrogen bond formation conditions with appropriate Pd catalyst (e.g., tBuXPhospd-G3) and ligand (e.g., tBuXPhos) under basic condition (e.g., t-BuONa) to yield compound A-7.

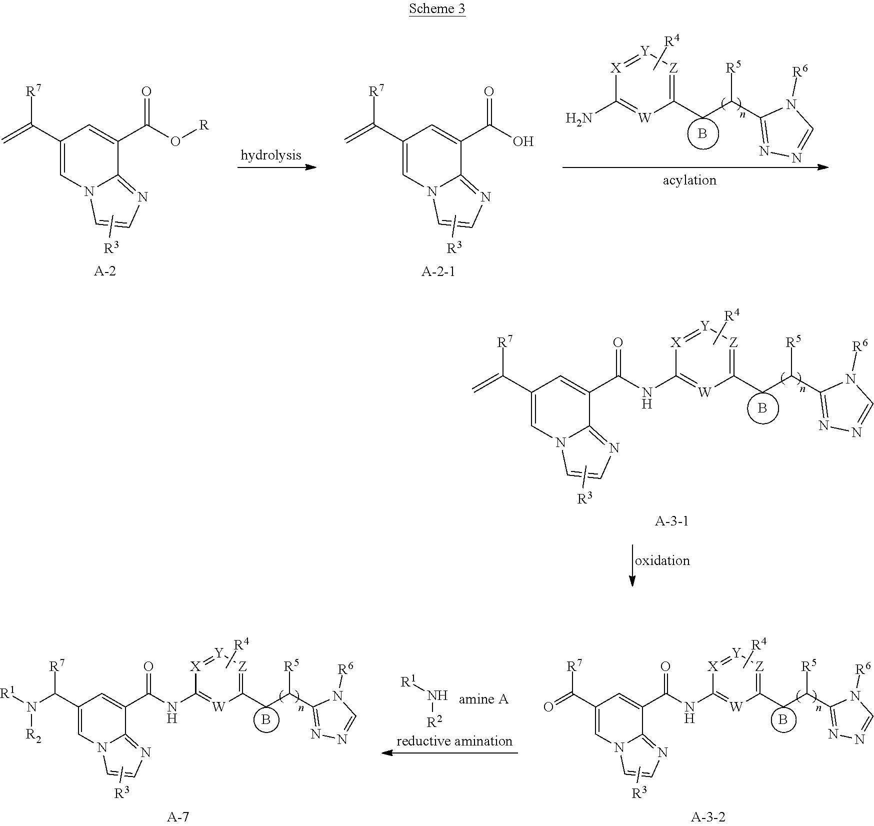

Scheme 3 outlines alternative preparation of compound A-7. Compound A-2-1 is prepared by hydrolysis compound A-2 in basic solution (e.g., KOH, NaOH or LiOH); then Compound A-2-1 is acylated with substituted aromatic amines under standard amide bond formation conditions (e.g., active ester formation; HATU or EDCI, DIPEA) to yield the compound A-3-1; then compound A-3-1 is oxidated (e.g., K₂OsO₄, NaIO₄) to give compound A-3-2; compound A-3-2 further undergoes reductive amination (e.g., STAB) with amine A to obtain compound A-7.

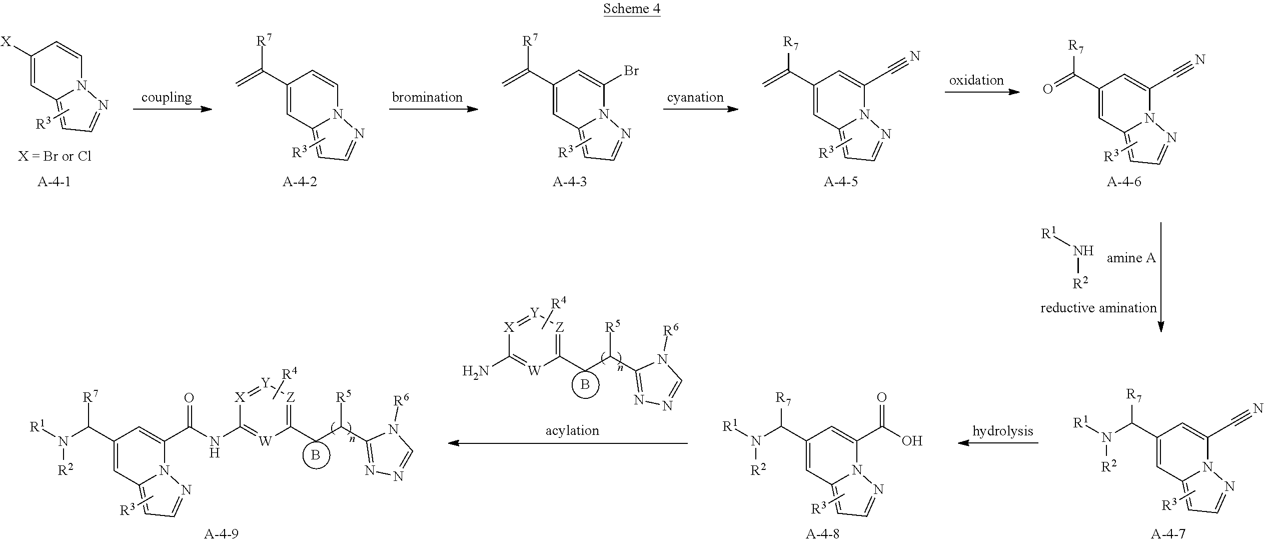

Scheme 4 illustrates an approach to synthesizing compound A-4-9. Halogen substituted compound A-4-1 is converted into compound A-4-2 in Suzuki coupling conditions (e.g., K₂CO₃, Pd(dppf)Cl₂); then compound A-4-2 is brominated to obtain compound A-4-3 under basic condition (e.g., TMPMgCl·LiCl, 1,2-dibromo-1,1,2,2-tetrachloroethane); compound A-4-3 is treated with cyanation agent (ZnCN) with appropriate Pd catalyst (e.g., Pd(OAc)₂,) and ligand (e.g., XantPhos) to form compound A-4-5; then compound A-4-5 is oxidated (e.g., K₂OsO₄, NaIO₄) to give compound A-4-6; reductive amination (e.g., STAB) of compound A-4-6 with amine A gives compound A-4-7; compound A-4-8 is prepared by hydrolysis of compound A-4-7 in basic solution (e.g., KOH, NaOH or LiOH) or acid solution (e.g., HCl); compound A-4-8 is acylated with substituted aromatic amines under standard amide bond formation conditions (e.g., active ester formation HATU or EDCI, DIPEA) to yield the compound A-4-9.

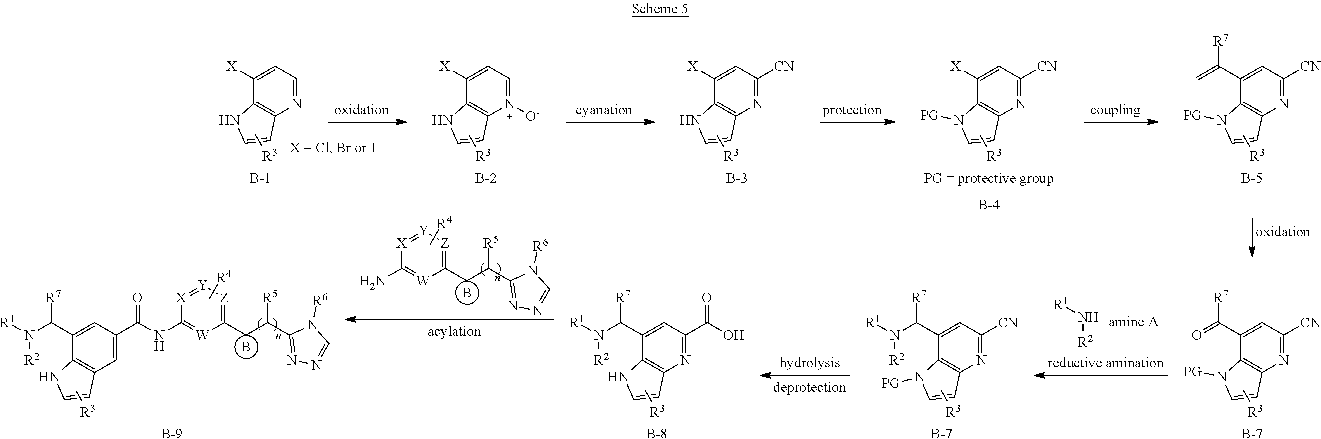

Scheme 5 shows an approach to synthesizing compound B-9. Halogen substituted compound B-1 is converted into compound B-2 under appropriate oxidation conditions (e.g., m-PCBA or H₂O₂); Compound B-2 is converted into corresponding compound B-3 by treatment with cyanation agent (such as TMSCN) under basic condition (e.g., Et₃N); Compound B-3 is reacted with appropriate protective group (e.g., SEM-, Boc-) under basic condition (e.g., NaH, DIPEA, Cs₂CO₃) to give Compound B-4; Compound B-5 is obtained by Suzuki coupling conditions (e.g., K₂CO₃, Pd(dppf)Cl₂.) with compound B-4; then compound B-5 is oxidated (e.g., K₂OsO₄, NaIO₄) to give compound B-6; compound B-6 further to undergo reductive amination (e.g., STAB) with amine A to obtain compound B-7. compound B-8 is prepared by hydrolysis and deprotection of compound B-7 under basic condition (e.g., KOH, NaOH or LiOH) or acid condition (e.g., HCl, or TFA); compound B-8 is acylated with substituted aromatic amines under standard amide bond formation conditions (e.g., active ester formation HATU or EDCI, DIPEA) to yield the compound B-9.

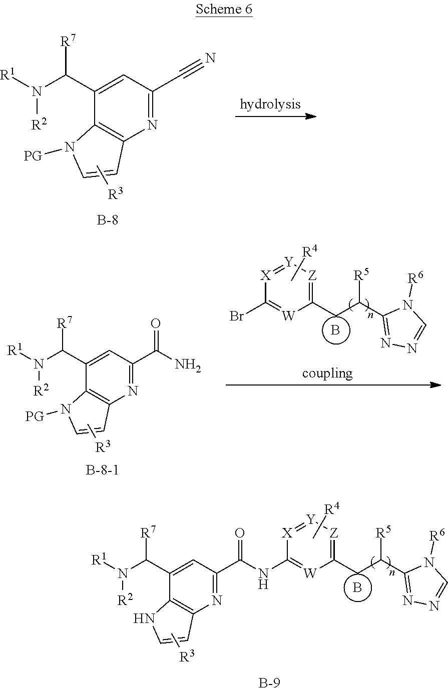

Scheme 6 outlines alternative preparation of compound B-9, Compound B-8 is hydrolyzed to give compound B-8-1 under basic condition (e.g., NaOH); then compound B-8-1 reacts with substituted aromatic halogen under standard carbon-nitrogen bond formation conditions with appropriate Pd catalyst (e.g., tBuXPhospd-G3) and ligand (e.g., tBuXPhos) under basic condition (e.g., t-BuONa) to yield compound B-9.

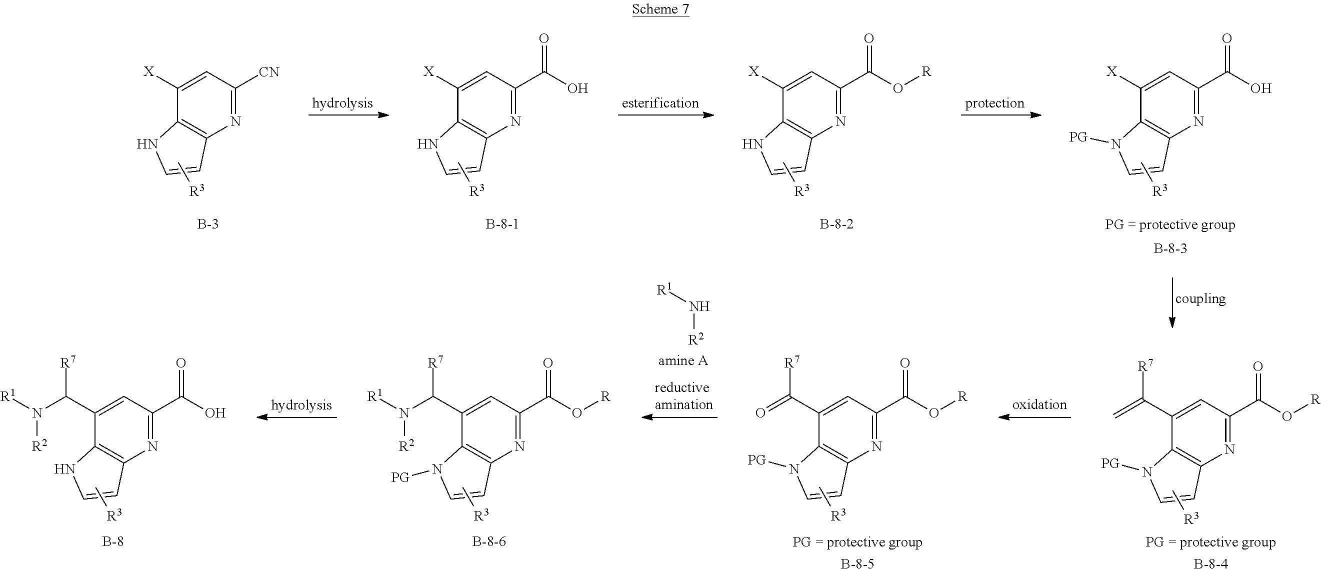

Scheme 7 outlines alternative preparation of compound B-8. Compound B-8-1 is prepared by hydrolysis of compound B-3 under basic condition (e.g., KOH, NaOH or LiOH) or acid condition (e.g., HCl, TFA); then esterified compound B-8-1 gives compound B-8-2 under acid condition (e.g., H₂SO₄); Compound B-8-2 is reacted with appropriate protective group (e.g., SEM-, Boc-) under basic condition (e.g., NaH, DIPEA, Cs₂CO₃) to give compound B-8-3; Compound B-8-4 is obtained by Suzuki coupling conditions (e.g., K₂CO₃, Pd(dppf)Cl₂) with compound B-8-3; then compound B-8-4 is oxidated (e.g., K₂OsO₄, NaIO₄) to give compound B-8-5; compound B-8-5 further undergoes reductive amination (e.g., STAB) with amine A to give compound B-8-6. Compound B-8 is prepared by hydrolysis and deprotection of compound B-8-6 under basic condition (e.g., KOH, NaOH or LiOH) or acid condition (e.g., HCl, or TFA).

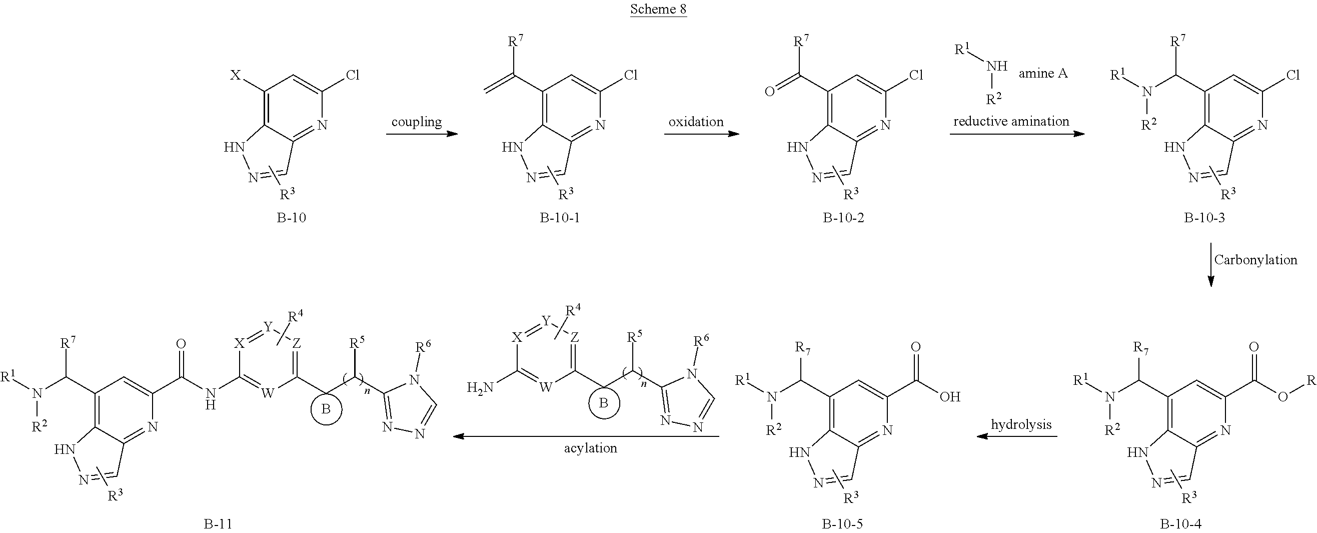

Scheme 8 illustrates an approach to synthesizing compound B-11. Compound B-10-1 is obtained by Suzuki coupling conditions (e.g., K₂CO₃, Pd(dppf)Cl₂.) with compound B-10; then compound B-10-1 is oxidated (e.g., K₂OsO₄, NaIO₄) to give compound B-10-2; compound B-10-2 further undergoes reductive amination (e.g., STAB) with amine A to give compound B-10-3. Then compound B-10-3 reacts with carbon monoxide with appropriate Pd catalyst (e.g., Pd(dppf)Cl₂) under basic condition (e.g., Et₃N) in solution (e.g., methanol, ethanol) to yield compound B-10-4; compound B-10-5 is prepared by hydrolysis of compound B-10-4 under basic condition (e.g., KOH, NaOH or LiOH) or acid condition (e.g., HCl, TFA); compound B-10-5 is acylated with substituted aromatic amines under standard amide bond formation conditions (e.g., active ester formation HATU or EDCI, DIPEA) to yield the compound B-11.

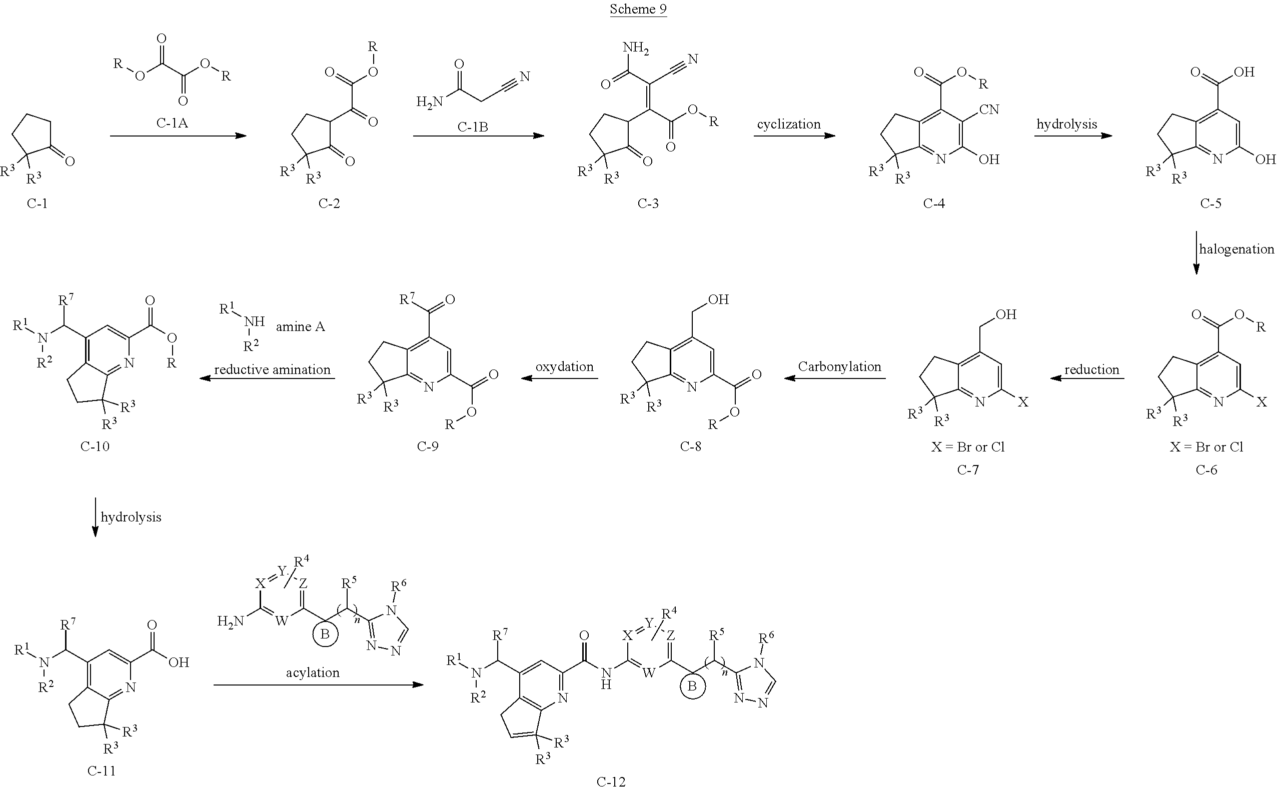

Scheme 9 outlines preparation of compound C-12. Under basic conditions (e.g., C₂H₅ONa, or CH₃ONa), treatment of compound C-1 with compound C-1A gives compound C-2. Compound C-2 reacts with compound C-1B to afford compound C-3, then via cyclization reaction to give compound C-4; compound C-5 is prepared by hydrolysis of compound C-4 under basic condition (e.g., KOH, NaOH or LiOH) or acid condition (e.g., HCl, TFA); The hydroxyl group of compound C-5 is halogenated by treatment with halogenation agent (e.g., POCl₃, or POBr₃) to give compound C-6; Reduction of Compound C-6 gives Compound C-7 as an alcohol using a reducing agent (such as NaBH4); then compound C-7 reacts with carbon monoxide with appropriate Pd catalyst (e.g., Pd(dppf)Cl₂) under basic condition (e.g., Et₃N) in solution (e.g., methanol, or ethanol) to yield compound C-8; compound C-8 is oxidated by oxidation agent (e.g., MnO₂, or DMP) to give compound C-9; compound C-9 further undergoes reductive amination (e.g., STAB) with amine A to give compound C-10; Compound C-11 is prepared by hydrolysis of compound C-10 under basic condition (e.g., KOH, NaOH or LiOH) or acid condition (e.g., HCl, TFA); compound C-11 is acylated with substituted aromatic amines under standard amide bond formation conditions (e.g., active ester formation HATU or EDCI, DIPEA) to yield the compound C-12.

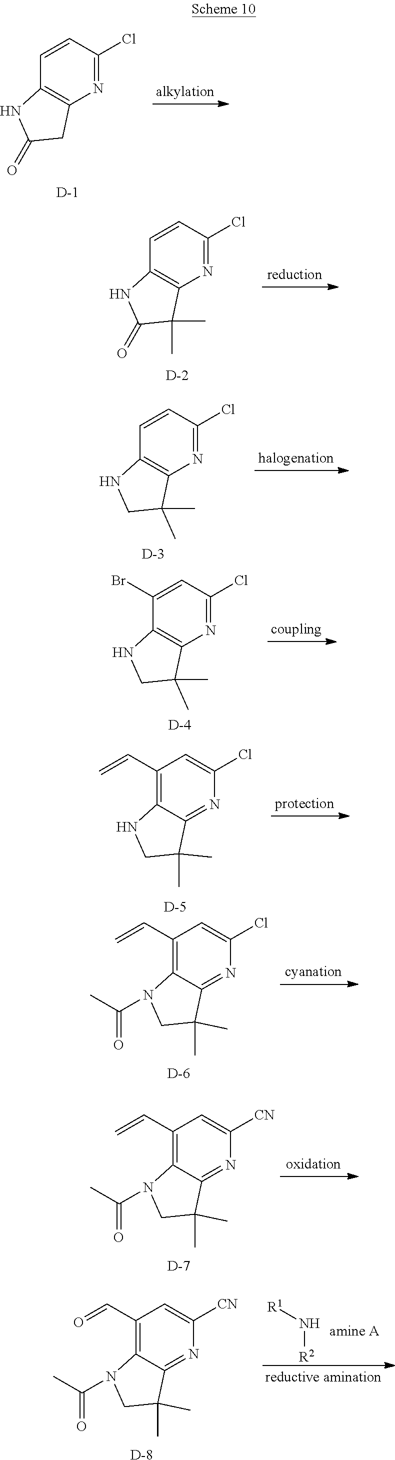

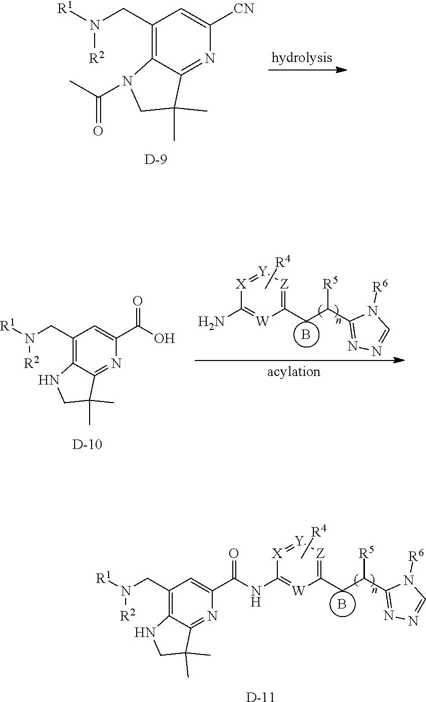

Scheme 10 illustrates an approach to synthesizing compound D-11. Compound D-2 is obtained by alkylation conditions (e.g., TMEDA/n-BuLi, NaH or LDA.) with alkyl halide; then compound D-2 is reduced (conditions e.g., BH3, LAH) to give compound D-3; compound D-3 further undergoes halogenation reaction conditions (e.g., NBS, Br2) to obtain the compound D-4, then compound D-4 is converted into compound D-5 in Suzuki coupling conditions (e.g., K₂CO₃, Pd(dppf)Cl₂); Compound D-5 is reacted with appropriate protective group (e.g., acetyl-, SEM-, Boc-) under basic condition (e.g., TEA, DMAP, NaH, DIPEA, Cs₂CO₃) to get compound D-6, Compound D-6 is treated with cyanation agent (ZnCN) with appropriate Pd catalyst (e.g., t-BuXphos G3, Zn, Pd(OAc)₂,) and ligand (e.g., XantPhos) to form compound D-7; then compound D-7 is oxidated (condition: e.g., K₂OsO₄, NaIO₄, NMO) to give compound D-8; compound D-8 further undergoes reductive amination (e.g., STAB, NaCNBH3) with amine A to give compound D-9; compound D-10 is prepared by hydrolysis of compound D-9 in basic solution (e.g., KOH, NaOH or LiOH) or acid solution (e.g., HCl, H2SO4); compound D-10 is acylated with substituted aromatic amines under standard amide bond formation conditions (e.g., active ester formation HATU or EDCI, DIPEA) to yield the compound D-11.

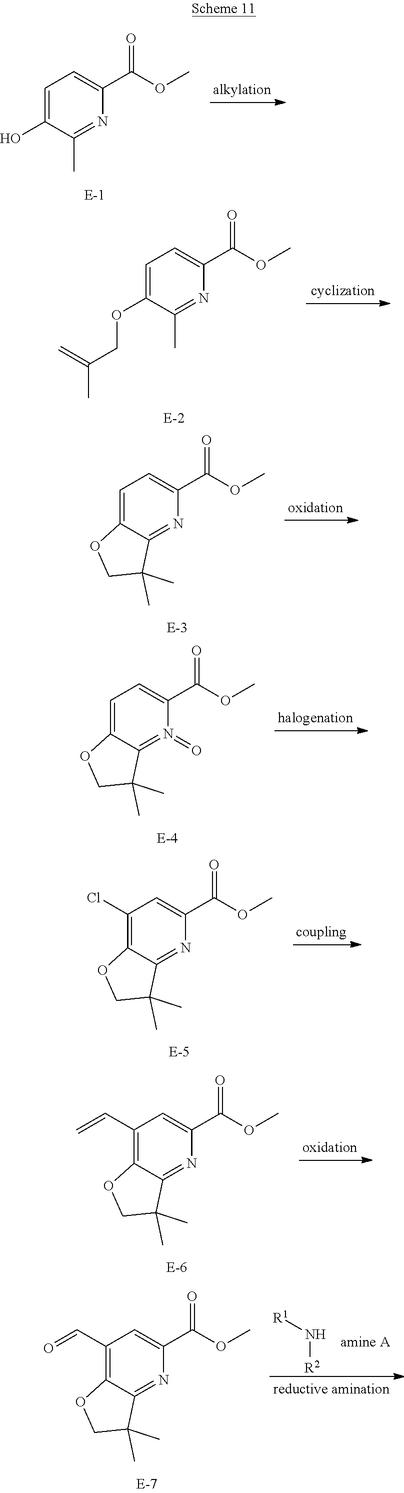

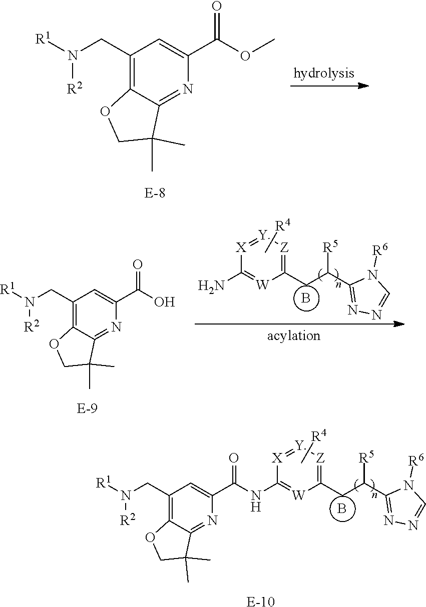

Scheme 11 outlines preparation of compound E-10. Compound E-2 is obtained by alkylation conditions (e.g., NaH or LDA.) treat E-1 with alkyl halide; Then E-2 undergo radical cyclization conditions (eg., Bu3SnH, AIBN,) to obtain the compound E-3; the compound E-3 is converted into compound E-4 under appropriate oxidation conditions (e.g., m-PCBA or H₂O₂); the compound E-4 is halogenated by treatment with halogenation agent (e.g., POCl₃, or POBr₃) to give compound E-5; Compound E-6 is obtained by Suzuki coupling conditions (e.g., K₂CO₃, Pd(dppf)Cl₂); then compound E-6 is oxidated (e.g., K₂OsO₄, NaIO₄) to give compound E-7; Compound E-7 further undergoes reductive amination (e.g., STAB) with amine A to give compound E-8; Compound E-9 is prepared by hydrolysis of compound E-8 under basic condition (e.g., KOH, NaOH or LiOH) or acid condition (e.g., HCl, TFA); compound E-9 is acylated with substituted aromatic amines under standard amide bond formation conditions (e.g., active ester formation HATU or EDCI, DIPEA) to yield the compound E-10.

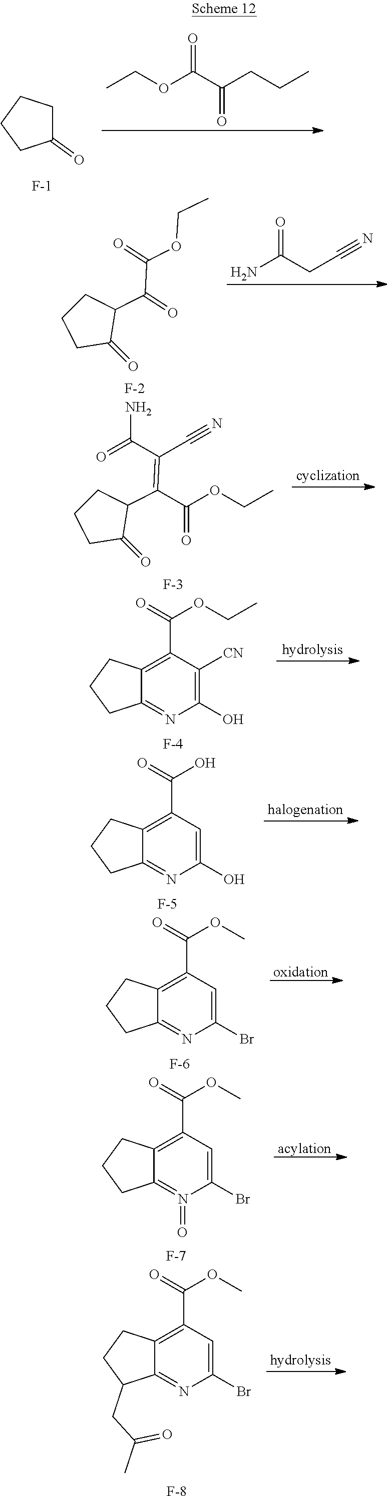

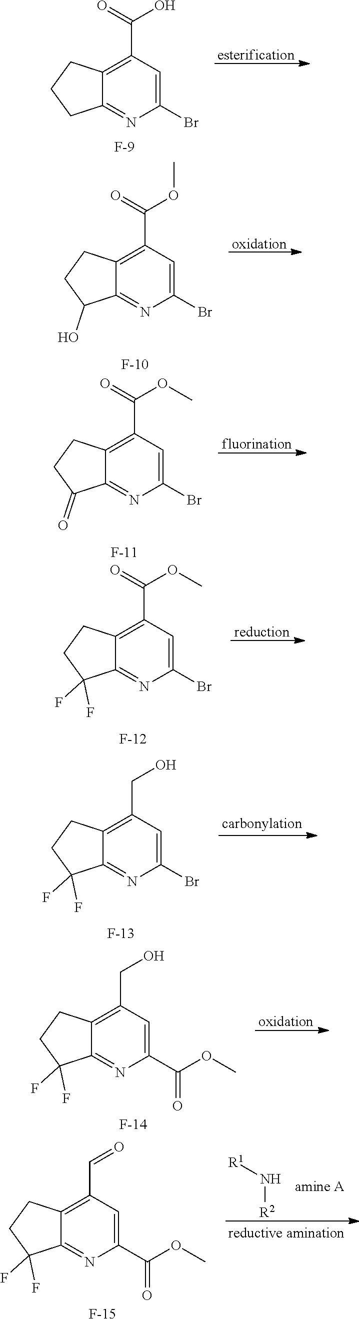

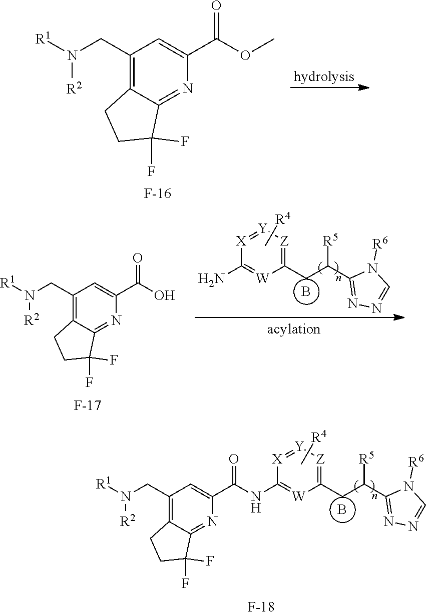

Scheme 12 outlines preparation of compound F-18. Compound F-4 is obtained three sequential steps conditions, treat F-4 with hydrolysis condition (e.g. HCl) to get compound F-5; Then F-5 undergo halogenation conditions (e.g., POCl₃, or POBr₃,) to obtain the compound F-6; the compound F-6 is converted into compound F-7 under appropriate oxidation conditions (e.g., m-PCBA or H₂O₂); the compound F-7 is acylation by treatment with Acetic Anhydride to give compound F-8; Compound F-9 is obtained by hydrolysis compound F-8 (conditions e.g., NaOH, LiOH.); F-9 undergo esterification to give F-10; then compound F-10 is oxidated (e.g., DMP, MnO₂) to give compound F-11; then by fluorination, reduction, carbonylation, and oxidation to give compound F-15; Compound F-15 further undergoes reductive amination (e.g., STAB) with amine A to give compound F-16; Compound F-17 is prepared by hydrolysis of compound F-16 under basic condition (e.g., KOH, NaOH or LiOH) or acid condition (e.g., HCl, TFA); compound F-17 is acylated with substituted aromatic amines under standard amide bond formation conditions (e.g., active ester formation HATU or EDCI, DIPEA) to yield the compound F-18.

The present embodiments can be understood more fully by reference to the detailed description and examples, which are intended to exemplify non-limiting embodiments.

### EXAMPLES

The examples below are intended to be purely exemplary and should not be considered to be limiting in any way. Unless otherwise specified, the experimental methods in the Examples described below are conventional methods. Unless otherwise specified, the reagents and materials are all commercially available. All solvents and chemicals employed are of analytical grade or chemical purity. Solvents are all redistilled before use. Anhydrous solvents are all prepared according to standard methods or reference methods. Silica gel (100-200 meshes) for column chromatography and silica gel (GF254) for thin-layer chromatography (TLC) are commercially available from Tsingdao Haiyang Chemical Co., Ltd. or Yantai Chemical Co., Ltd. of China; all were eluted with petroleum ether (60-90° C.)/ethyl acetate (v/v), and visualized by iodine or the solution of molybdphosphoric acid in ethanol unless otherwise specified. All extraction solvents, unless otherwise specified, were dried over anhydrous Na₂SO₄. ¹H NMR spectra were recorded on Bruck-400, Varian 400MR nuclear magnetic resonance spectrometer with TMS (tetramethylsilane) as the internal standard. Coupling constants were given in hertz. Peaks were reported as singlet (s), doublet (d), triplet (t), quartet (q), quintet (p), sextet (h), septet (hept), multiplet (m), or a combination thereof; br stands for broad. LC/MS data was recorded by using Agilent 1100,1200 High Performance Liquid Chromatography-Ion Trap Mass Spectrometer (LC-MSD Trap) equipped with a diode array detector (DAD) detected at 214 nm and 254 nm, and an ion trap (ESI source). All compound names except the reagents were generated by ChemDraw® 18.0. or Chemaxon-Marvin JS. All the mobile phase ratio of prep-HPLC was demonstrated by B % (organic phase), and corresponding aqueous phase was A %=100%−B %.

In the following examples, the following abbreviations are used:
AcOH Acetic acid
Aq. Aqueous
BINAP 2,2′-bis(diphenylphosphino)-1,1′-binaphthalene
Brine Saturated aqueous sodium chloride solution
Bn Benzyl
BnBr Benzyl Bromide
Boc Tert-butoxycarbonyl
CH₂Cl₂ or DCM Dichloromethane
CAN Cerium(IV) ammonium nitrate (cericammonium nitrate)
DAST Diethylaminosulfur trifluoride
DMF N,N-Dimethylformamide
Dppf 1,1′-bis(diphenylphosphino)ferrocene
DBU 1,8-diazabicyclo[5.4.0]undec-7-ene
DHP 3,4-Dihydro-2H-pyran
DIEA or DIPEA N,N-diisopropylethylamine
DMAP 4-N,N-dimethylaminopyridine
DMB (2,4-dimethoxyphenyl) methanamine
Dess-Martin/DMP Dess-Martin Periodinane
DMF N,N-dimethylformamide
DMF-DMA N,N-Dimethylformamide dimethyl acetal purum
DMSO Dimethyl sulfoxide
DMEDA Dimethyl Ethylene Diamine
EDCI 1-Ethyl-3-(3-dimethylaminopropyl)carbodiimide hydrochloride
EtOAc or EA Ethyl acetate
EtOH Ethanol
Et₃SiH Triethyl silane
Et₂O or ether Diethyl ether
g Grams
h or hr Hour
HATU O-(7-Azabenzotriazol-1-yl)-N,N,N′,N′-tetramethyluronium hexafluorophosphate
Hex Hexane
HCl Hydrochloric acid
HMDS Hexamethyldisilazane
HOBT 1-Hydroxybenzotriazole
HPLC High-performance liquid chromatography
IBX 2-Iodylbenzoic acid
i-PrOH Isopropyl alcohol
LCMS Liquid chromatography-mass spectrometry
LDA Lithium diisopropylamide
LiHMDS Lithium Bis(trimethylsilyl)amide
K₂OsO₄·H2O Potassium osmate (VI) dihydrate
mg Milligrams
mL Milliliters
mmol Millimole
MeCN Acetonitrile
MeOH Methanol
Min Minutes
ms or MS Mass spectrum
m-CPBA 2-chloranylbenzenecarboperoxoic acid
MPLC Medium Pressure Liquid Chromatography
Na₂SO₄ Sodium sulfate
NaBH(OAc)₃/STAB Sodium triacetyl borohydride
NaHMDS Sodium bis(trimethylsilyl)amide
NBS N-Bromosuccinimide
NCS N-Chlorosuccinimide
NMO 4-Methylmorpholine N-oxide
NMP N-Methyl Pyrrolidone
PE petroleum ether
PMB (4-methoxyphenyl)methanamine
POCl₃ phosphorous oxychloride
PyBOP Benzotriazol-1-yl-oxytripyrrolidinophosphonium hexafluorophosphate [1,1′-Bis(diphenylphosphino)ferrocene]dichloropalladium(II)
PddppfCl₂ Tris(dibenzylideneacetone)dipalladium
Pd₂(dba)₃
Prep Preparative
PTSA 4-Methylbenzenesulfonic acid
Rt or rt Room temperature
sat. Saturated
SEMCl (2-(Chloromethoxy)ethyl)trimethylsilane
TBSCl tert-Butyldimethylsilyl chloride
TEA/Et3N triethylamine
t-BuOK Potassium tert-butoxide
t-BuONa Sodium tert-butoxide
T₃P n-Propylphosphonic cyclic anhydride
TMSCN Trimethylsilyl cyanide
TFA Trifluoroacetic acid
TFAA Trifluoroacetic anhydride
THF Tetrahydrofuran
TLC thin layer chromatography
tBuXPhospd-G3 Methanesulfonato(2-di-t-butylphosphino-2′,4′,6′-tri-i-propyl-1,1′-biphenyl)(2′-amino-1,1′-biphenyl-2-yl)palladium(II)
tBuXPhos 2-Di-tert-butylphosphino-2′,4′,6′-triisopropylbiphenyl
UHP Urea hydrogen peroxide
Ml Microliters
XantPhos 4,5-Bis(diphenylphosphino)-9,9-dimethylxanthene
XPhos 2-Dicyclohexylphosphino-2′,4′,6′-triisopropylbiphenyl
4CzIPN (4r, 6r)-2,4,5,6-tetra(9H-carbazol-9-yl)isophthalonitrile

### Intermediate Compound Synthesis

### Compound INT1: (3R,4S)-4-fluoro-3-methylpiperidine, HCl

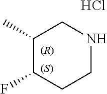

### Step 1: (R)-1-benzyl-3-methylpiperidin-4-one DTTA

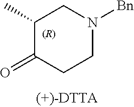

Charge 1-benzyl-3-methyl-piperidin-4-one (600 g, 2.95 mol, 1.00 eq) and ACN (3.00 L) into the reactor R-1 at 20-25° C. under N₂. The reaction mixture was warming to 40-45° C. Charge (2S,3S)-2,3-bis [(4-methylbenzoyl)oxy]butanedioic acid (1.37 kg, 3.54 mol, 1.20 eq) into the reactor R-1 at 40-45° C. The reaction mixture was stirred at 40-45° C. for 18 hrs. The reaction mixture was filter and filter cake was collected. The filter cake was washed with MeCN (1.00 L). to give (3R)-1-benzyl-3-methyl-piperidin-4-one DTTA (1.20 kg, crude).

### Step 2: (R)-1-benzyl-3-methylpiperidin-4-one

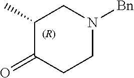

Charge 1-benzyl-3-methyl-piperidin-4-one DTTA (1.20 kg, 1.00 eq) and DCM (8.40 L) into the reactor R-1 at 20-25° C. Charge H₂O (2.40 L) into the reactor R-1 at 20-25° C. The reaction mixture was stirred at 20-25° C. for 0.5 hr. Charge NH₃H₂O (500 mL) and the reaction mixture adjust the pH=10. Extracted with DCM (500 ml) and the organic phase was collected. The organic phase was concentrated in vacuum. To give (3R)-1-benzyl-3-methyl-piperidin-4-one (410 g, 2.02 mol, 68.3% yield).

### Step 3: tert-butyl (R)-3-methyl-4-oxopiperidine-1-carboxylate

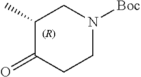

Set up the reactor R-1 (250 ml). Charge Pd/C (6.5 g, 639 mmol, 10% purity, 1.00 eq) and EtOAc (910 mL) into the reactor R-1 at 15-20° C. under argon (Ar). Charge (3R)-1-benzyl-3-methyl-piperidin-4-one (130 g, 639 mmol, 1.00 eq) into the reactor R-1 at 15-25° C. Charge Boc₂O (167 g, 767 mmol, 176 mL, 1.20 eq) into the reactor R-1 at 15-25° C. The suspension was degassed under vacuum and purged with H₂ three times. The mixture was stirred under H₂ (50 psi) at 15-25° C. for 6 hrs. LCMS showed the reaction was consumed completely. 3 reactions were together for work up. The reaction mixture was filter and the filter liquor was collected. The filter liquor was concentrated in vacuum. The crude product dissolution completely with n-heptane (200 mL). Cool to 0-5° C. the reaction mixture was stirred for 2 hrs. The reaction mixture was filter and the filter cake was collected and concentrated in vacuum. To give tert-butyl (3R)-3-methyl-4-oxo-piperidine-1-carboxylate (200 g, 937 mmol, 48.8% yield, 100% purity).

### Step 4: tert-butyl (3R)-4-hydroxy-3-methylpiperidine-1-carboxylate

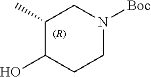

Set up the reactor R-1 (2.00 L)(2 batches) Charge tert-butyl (3R)-3-methyl-4-oxo-piperidine-1-carboxylate (100 g, 468 mmol, 1.00 eq) and MeOH (700 mL) into the reactor R-1 at 15-20° C. unde N₂. Cool to 0-5° C., charge NaBH4 (26.6 g, 703 mmol, 1.50 eq) into the reactor R-1 under N₂ The reaction mixture was stirred at 0-5° C. for 1 hr. The reaction mixture was stirred at 20-25° C. for 2 hrs. LCMS showed the reaction was consumed completely. The reaction mixture was quenched with saturation NH₄Cl (100 ml). The reaction mixture was concentrated in vacuum. Extracted with DCM (200 mL) and the organic phase was collected. The organic phase was concentrated in vacuum. Give tert-butyl (3R)-4-hydroxy-3-methylpiperidine-1-carboxylate was obtained (184 g, 93.5% purity).

### Step 5: tert-butyl (3R,4R)-4-hydroxy-3-methylpiperidine-1-carboxylate

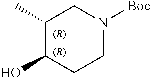

The crude product (184 g, 93.5% purity) was purified by SFC separate (column: DAICEL CHIRALPAK AD (250 mm*50 mm, 10 um); mobile phase: [0.1% NH₃H₂O ETOH]; B %: 13%-13%, 3.5 min). Give tert-butyl (3R,4S)-4-hydroxy-3-methyl-piperidine-1-carboxylate (85.0 g, 394 mmol, 46.2% yield, 100% purity).

### Step 6: tert-butyl (3R,4R)-3-methyl-4-(tosyloxy) piperidine-1-carboxylate

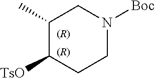

Set up the reactor R-1 (1.00 L). Charge tert-butyl (3R,4R)-4-hydroxy-3-methyl-piperidine-1-carboxylate (80.0 g, 371 mmol, 1.00 eq) and pyridine (560 mL) into the reactor R-1 at 20-25° C. Charge TsCl (92.1 g, 483 mmol, 1.30 eq) into the reactor R-1 under N₂. The traction mixture was stirred at 20-25° C. for 12 hrs. LCMS showed the reaction was consumed completely. The reaction mixture was quenched with H₂O (200 ml) at 15-20° C. Extracted with DCM (300 mL) and the organic phase was collected. The organic phase was washed with HCl (0.5 M aqueous solution 500 mL×2), adjust to pH=6. Extracted with DCM (200 mL) and the organic phase was collected. The organic phase was concentrated in vacuum. The crude product was triturated with n-heptane (100 mL) at 15-20° C. for 1 hr. The reaction mixture was filter and the filter cake was collected. The filter cake was dryed at 40-45° C. for 4 hrs. Give tert-butyl (3R,4R)-3-methyl-4-(p-tolylsulfonyloxy) piperidine-1-carboxylate (100 g, 270 mmol, 72.8% yield).

### Step 7: tert-butyl (3R,4S)-4-fluoro-3-methylpiperidine-1-carboxylate

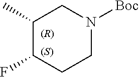

Set up the reactor R-1 (2.00 L). Charge tert-butyl (3R,4R)-3-methyl-4-(p-tolylsulfonyloxy) piperidine-1-carboxylate (100 g, 270 mmol, 1.00 eq) into the reactor R-1 at 15-20° C. Charge tetrabutylammonium; fluoride; trihydrate (1 M, 1.35 L, 5.00 eq) into the reactor R-1 at 15-20° C. The reaction mixture was stirred at 60-65° C. for 8 hrs. HPLC and TLC (Petroleum ether:EtOAc=8:1, Rf=0.65) showed the reaction was consumed completely, and the product was detected. Cool to 15-20° C., the reaction mixture was quenched with H₂O (100 mL). Extracted with DCM (200 mL) and the organic phase was collected. The organic phase was concentrated in vacuum. The crude product was purified by column chromatography (SiO₂, Petroleum ether/Ethyl acetate=60/1 to 20/1). Give tert-butyl (3R,4S)-4-fluoro-3-methyl-piperidine-1-carboxylate (20.0 g, 92.0 mmol, 34.0% yield).

### Step 8: (3R,4S)-4-fluoro-3-methylpiperidine, HCl

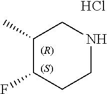

Set up the reactor R-1 (100 mL). Charge tert-butyl (3R,4S)-4-fluoro-3-methyl-piperidine-1-carboxylate (20.0 g, 92.0 mmol, 1.00 eq) and HCl/EtOAc (4 M solution, 140 mL) into the reactor R-1 at 20-25° C. The traction mixture was stirred at 20-25° C. for 1 hr. LCMS and TLC (Petroleum ether:EtOAc=8:1) showed the reaction was consumed completely, and the product was detected. The reaction mixture was concentrated in vacuum. The crude product was triturated with n-heptane (40.0 mL) at 25° C. for 1 hr. The reaction mixture was filter and the filter cake was collected. The filter cake was drying at 40-45° C. for 2 hrs. Give (3R,4S)-4-fluoro-3-methyl-piperidine (11.0 g, 71.60 mmol, 77.79% yield, 100% purity, HCl).

### Compound INT2: 3-[3-[(4-methyl-1,2,4-triazol-3-yl)methyl]oxetan-3-yl]aniline

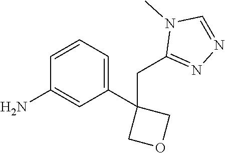 [SMILES: Cn1cnnc1CC1(c2cccc(N)c2)COC1]

### Step 1: ethyl 2-(oxetan-3-ylidene)acetate

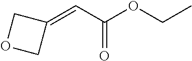

To a solution of ethyl 2-(triphenyl-λ⁵-phosphanylidene) acetate (406 g, 1.17 mol, 1.05 eq) in DCM (400 mL) was added a solution of oxetan-3-one (80.0 g, 1.11 mol, 1.00 eq) in DCM (200 mL) at 0-10° C. The mixture was stirred at 20° C. for 2 hrs. The mixture was concentrated under reduced pressure to give a residue. The residue was triturated with petroleum ether (500 mL) at 25° C. for 2 hrs. ethyl 2-(oxetan-3-ylidene)acetate (250 g 1.76 mol, 79.2% yield) was obtained

### Step 2: ethyl 2-(3-(3-nitrophenyl)oxetan-3-yl)acetate

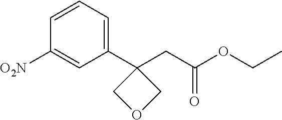 [SMILES: CCOC(=O)CC1(c2cccc([N+](=O)[O-])c2)COC1]

To a solution of ethyl 2-(oxetan-3-ylidene)acetate (20.0 g, 140 mmol, 1.00 eq) in dioxane (80.0 mL) was added chlororhodium; (1Z,5Z)-cycloocta-1,5-diene (6.74 g, 14.03 mmol, 0.1 eq) and KOH (1.50 M, 140 mL, 1.50 eq) in H₂O (20.0 mL), then (3-nitrophenyl) boronic acid (30.5 g, 182 mmol, 1.30 eq) was added in small portions. The mixture was stirred at 15° C. for 4 hrs. extracted the mixture between the two phases of EtOAc (200 mL×3) and H₂O (300 mL) after filtered through celite, separated and washed the organic layer with brine (100 mL). The organic layer was dried over Na₂SO₄ and filtered, concentrated to dryness in vacuum. The crude was purified by column chromatography on silica gel (Petroleum ether/Ethyl acetate=10/1 to 1/1). ethyl 2-[3-(3-nitrophenyl)oxetan-3-yl]acetate (10 g) was obtained; ¹H NMR: (400 MHz, CDCl₃-d) δ ppm 8.15 (m, 1H), 8.10-8.03 (m, 1H), 7.67-7.50 (m, 2H), 5.02 (d, J=6.4 Hz, 2H), 4.91 (d, J=6.4 Hz, 2H), 4.04 (q, J=7.2 Hz, 2H), 3.20 (s, 2H), 1.16 (t, J=7.2 Hz, 3H) ppm; MS: M/e 266 (M+1)⁺.

### Step 3: 2-(3-(3-nitrophenyl)oxetan-3-yl)acetohydrazide

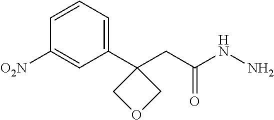 [SMILES: NNC(=O)CC1(c2cccc([N+](=O)[O-])c2)COC1]

To a solution of ethyl 2-[3-(3-nitrophenyl)oxetan-3-yl]acetate (40.0 g, 150 mmol, 1.00 eq) in EtOH (200 mL) was added N₂H₄·H₂O (49.1 g, 961 mmol, 47.6 mL, 98.0% purity, 6.38 eq). The mixture was stirred at 80° C. for 16 hrs. TLC (Dichloromethane/Methanol=10/1, R_{f} (2-(3-(3-nitrophenyl)oxetan-3-yl) aceto hydrazide)=0.20, I₂) indicated 60% of reactant 1 was not consumed. Then the mixture was added N₂H₄·H₂O (24.3 g, 477 mmol, 23.6 mL, 98% purity, 3.16 eq) at 25° C. The mixture was stirred at 80° C. for 16 hrs. TLC (Dichloromethane/Methanol=10/1, R_{f}(2-(3-(3-nitrophenyl)oxetan-3-yl) aceto hydrazide)=0.20, I₂) indicated all of reactant was consumed. The mixture was concentrated under reduced pressure to give 2-[3-(3-nitrophenyl)oxetan-3-yl]acetohydrazide (32.0 g).

### Step 4: 1-methyl-3-[[2-[3-(3-nitrophenyl)oxetan-3-yl]acetyl]amino]thiourea

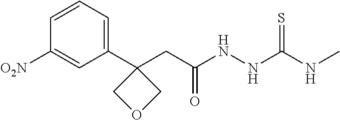 [SMILES: CNC(=S)NNC(=O)CC1(c2cccc([N+](=O)[O-])c2)COC1]

To a solution of 2-[3-(3-nitrophenyl)oxetan-3-yl]acetohydrazide (75.0 g, 298 mmol, 1.00 eq) in THF (450 mL) was added methylimino(thioxo)methane (43.6 g, 597 mmol, 40.8 mL, 2.00 eq). The mixture was stirred at 70° C. for 5 hrs. The mixture was concentrated to get the crude product. The crude product (80.0 g) was obtained and used to next step without further purification.

### Step 5: 4-methyl-5-[[3-(3-nitrophenyl)oxetan-3-yl]methyl]-1,2,4-triazole-3-thiol

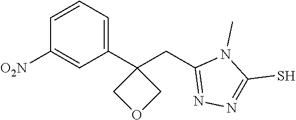 [SMILES: Cn1c(S)nnc1CC1(c2cccc([N+](=O)[O-])c2)COC1]

To a solution of 1-methyl-3-[[2-[3-(3-nitrophenyl)oxetan-3-yl]acetyl]amino]thiourea (80.0 g, 246 mmol, 1.00 eq) in NaOH (1 M, 2.47 L, 10.0 eq). The mixture was stirred at 25° C. for 8 hrs. The reaction was diluted with water, then the pH value of the solution was adjusted to 5 with HCl (1 N, 2.00 L) and extracted with EtOAc (1.00 L×3). The combined organic layers were washed with brine 800 mL, dried over Na₂SO₄, filtered and concentrated under reduced pressure to give a residue. 4-methyl-5-[[3-(3-nitrophenyl)oxetan-3-yl]methyl]-1,2,4-triazole-3-thiol (90.0 g, crude) was obtained.

### Step 6: 4-methyl-3-[[3-(3-nitrophenyl)oxetan-3-yl]methyl]-1,2,4-triazole

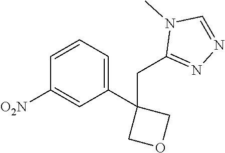 [SMILES: Cn1cnnc1CC1(c2cccc([N+](=O)[O-])c2)COC1]

To a solution of 4-methyl-5-[[3-(3-nitrophenyl)oxetan-3-yl]methyl]-1,2,4-triazole-3-thiol (30.0 g, 97.9 mmol, 1.00 eq) in THF (450 mL) and H₂O (450 mL) was added NaNO₂ (67.5 g, 979 mmol, 4.90 mL, 10.0 eq) and HNO₃ (94.9 g, 979 mmol, 67.8 mL, 65% purity, 10.0 eq) at 0° C. The mixture was basified by saturated NaHCO₃ aqueous (0.5 L) and was diluted by the addition of water and extracted with EtOAc (200 mL×4), organic phase was combined and concentrated to give a residue. The residue was purified by column chromatography (SiO₂, dichloromethane/Methanol=100/1 to 0/1). 4-methyl-3-[[3-(3-nitrophenyl)oxetan-3-yl]methyl]-1,2,4-triazole (20 g) was obtained; ¹HNMR: (400 MHz, CDCl₃-d) δ ppm 8.18-8.05 (m, 1H), 7.98-7.85 (m, 2H), 7.46 (t, J=8.0 Hz, 1H), 7.35 (m, 1H), 5.18-4.99 (m, 4H), 3.58 (s, 2H), 3.11 (s, 3H) ppm; MS: M/e 275 (M+1)⁺.

### Step 7: 3-[3-[(4-methyl-1,2,4-triazol-3-yl)methyl]oxetan-3-yl]aniline

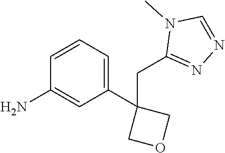 [SMILES: Cn1cnnc1CC1(c2cccc(N)c2)COC1]

To a solution of 4-methyl-3-[[3-(3-nitrophenyl)oxetan-3-yl]methyl]-1,2,4-triazole (10.0 g, 36.4 mmol, 1.00 eq) in EtOH (100 mL) and H₂O (30.0 mL) was added NH₄Cl (9.75 g, 182 mmol, 5.00 eq). Then the mixture was added Fe (6.11 g, 109 mmol, 3.00 eq) at 80° C. The mixture was stirred at 80° C. for 2 hrs. Four batches were combined to work up. The mixture was filtered and the filtrate was concentrated under reduced pressure to give a residue. The reaction was diluted with water (100 mL), then the pH value of the solution was adjusted to 8 with NaHCO₃ (150 mL) and extracted with Dichloromethane/Methanol=10/1 (200 mL×3). The combined organic layers were washed with brine 100 mL, dried over Na₂SO₄, filtered and concentrated under reduced pressure to give a residue. The crude product was purified by column chromatography (SiO₂, dichloromethane/Methanol=100/1 to 0/1) to obtain 3-[3-[(4-methyl-1,2,4-triazol-3-yl)methyl]oxetan-3-yl]aniline (31.4 g); ¹H NMR: (400 MHz, CDCl₃-d) δ ppm 8.20 (s, 1H), 6.89 (t, J=7.6 Hz, 1H), 6.40 (br d, J=7.6 Hz, 1H), 6.04 (s, 1H), 5.94 (br d, J=7.2 Hz, 1H), 5.02 (br s, 2H), 4.84 (d, J=5.6 Hz, 2H), 4.75 (d, J=5.6 Hz, 2H), 3.39 (s, 2H), 2.83 (s, 3H) ppm; MS: M/e 245 (M+1)⁺.

### Compound INT3: (1r, 3r)-3-(3-aminophenyl)-3-((4-methyl-4H-1,2,4-triazol-3-yl)methyl)cyclobutane-1-carbonitrile

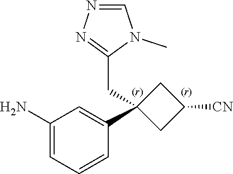 [SMILES: Cn1cnnc1C[C@]1(c2cccc(N)c2)C[C@H](C#N)C1]

### Step 1: methyl 2-(3-cyanocyclobutylidene)acetate

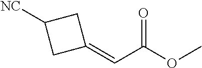

To a solution of NaH (13.9 g*6, 347 mmol, 60%) in THF (900 mL) was added methyl 2-(dimethoxyphosphoryl)acetate (57.5 g*6, 315.5 mmol) at 0° C. under N₂. The mixture was stirred for 1 hrs at 25° C. and then added 3-oxocyclobutane-1-carbonitrile (30 g*6, 315.5 mmol) in THF (90 mL) at 0° C. under N₂. The mixture was stirred for 13 hrs at 25° C. The mixture was poured into sat.NH₄Cl aqueous (1000 ml) and extracted with EtOAc (1000 mL, 500 mL). The combined organic phase was washed with brine (100 ml), dried over Na₂SO₄, filtered and concentrated to give the residue, which was purified by column chromatography (SiO₂, PE:EA=100/1, 0/1) to give the product (192.0 g, 67% yield). MS: M/e 152 (M+1)⁺.

### Step 2: methyl 2-((1r, 3r)-1-(3-((tert-butoxycarbonyl)amino)phenyl)-3-cyanocyclobutyl)acetate

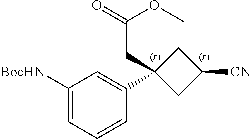 [SMILES: COC(=O)C[C@]1(c2cccc(NC(=O)OC(C)(C)C)c2)C[C@H](C#N)C1]

To a solution of methyl 2-(3-cyanocyclobutylidene)acetate (75.0*2 g, 496 mmol) in dioxane (750 mL) was added (3-((tert-butoxycarbonyl)amino)phenyl)boronic acid (176 g*2, 744 mmol), KOH (430*2 ml, 545 mmol, 1.5 M) and chloro(1,5-cyclooctadiene)rhodium(I) dimer (CAS: 12092-47-6) (24.5 g*2, 49.6 mmol) at 25° C. under N₂. The mixture was stirred for 15 h at 25° C. under N₂. The mixture was poured into water (800 mL) and extracted with EtOAc (1000 mL, 500 mL). The combined organic phase was washed with brine (500 mL), dried over Na₂SO₄, filtered and concentrated to give the residue, which was purified by column chromatography (SiO₂, PE:EA=100/1, 0/1) to give methyl 2-((1r, 3r)-1-(3-((tert-butoxycarbonyl)amino)phenyl)-3-cyanocyclobutyl)acetate (50 g, 14.6% yield) and methyl 2-(1-(3-((tert-butoxycarbonyl)amino)phenyl)-3-cyanocyclobutyl)acetate (98 g, CIS:TRANS=1:1). MS: M/e 345 (M+1)+.

### Step 3: 2-((1r, 3r)-1-(3-((tert-butoxycarbonyl)amino)phenyl)-3-cyanocyclobutyl)acetic acid

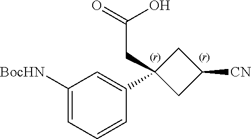 [SMILES: CC(C)(C)OC(=O)Nc1cccc([C@]2(CC(=O)O)C[C@@H](C#N)C2)c1]

To a solution of methyl 2-((1r, 3r)-1-(3-((tert-butoxycarbonyl)amino)phenyl)-3-cyanocyclobutyl)acetate (46.0 g, 133.6 mmol) in THF/MeOH/H₂O (450 ml, 1:1:1) was added LiOH·H₂O (16.8 g, 400.7 mmol) at 20° C. The mixture was stirred for 12 hrs at 20° C. under N₂. The mixture was adjusted to pH=6.0 by HCl (1 M) and extracted with EtOAc (900 mL, 500 mL). The combined organic phase was washed with brine (200 mL), dried over Na₂SO₄, filtered and concentrated to give the residue, which was purified by column chromatography (SiO₂, PE:EA=100/1, 0/1) to give the product (35 g, 80% yield). MS: M/e 329 (M−1)⁻. ¹H NMR: (400 MHz, CDCl₃-d) δ 7.24-7.27 (m, 4H) 6.89-6.91 (m, 1H) 2.89-2.30 (m, 5H) 2.65-2.78 (m, 2H) 1.51 (s, 9H).

### Step 4: tert-butyl (3-((1r, 3r)-3-cyano-1-((5-mercapto-4-methyl-4H-1,2,4-triazol-3-yl)methyl)cyclobutyl)phenyl)carbamate

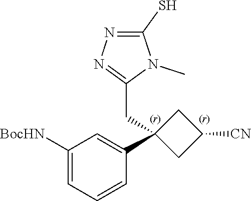 [SMILES: Cn1c(S)nnc1C[C@]1(c2cccc(NC(=O)OC(C)(C)C)c2)C[C@H](C#N)C1]

To a solution of 2-((1r, 3r)-1-(3-((tert-butoxycarbonyl)amino)phenyl)-3-cyanocyclobutyl)acetic acid (35 g, 106 mmol) in DMF (350 mL) was DIEA (15.1 g, 116.5 mmol), HATU (52.4 g, 137.7 mmol) and N-methylhydrazinecarbothioamide (16.7 g, 159.0 mmol) at 20° C. The mixture was stirred for 13 hrs at 25° C. and then added NaOH (222.5 mL, 222.5 mmol, 1M). The mixture was stirred for 12 hrs at 60° C. The mixture was poured into water (500 mL) and adjusted to pH=6.0 by HCl (1M) and extracted with EtOAc (500 mL, 300 mL). The combined organic phase was washed with brine (200*2 mL) and dried over Na₂SO₄, filtered and concentrated to give the residue, which was purified by column chromatography (SiO₂, PE:EA=100/1, 0/1) to give the product (36 g, 86% yield). MS: M/e 400 (M+1)⁺.

### Step 5: tert-butyl (3-((1r, 3r)-3-cyano-1-((4-methyl-4H-1,2,4-triazol-3-yl)methyl)cyclobutyl)phenyl)carbamate

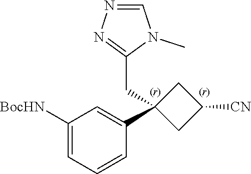 [SMILES: Cn1cnnc1C[C@]1(c2cccc(NC(=O)OC(C)(C)C)c2)C[C@H](C#N)C1]

To a solution of tert-butyl (3-((1r, 3r)-3-cyano-1-((5-mercapto-4-methyl-4H-1,2,4-triazol-3-yl)methyl)cyclobutyl)phenyl)carbamate (36 g, 90.1 mmol) in DCM (720 ml) was added AcOH (72 ml) and H₂O₂(40.8 g, 360.1 mmol, 30%) at 0° C. The mixture was stirred for 6 hrs at 20° C. The mixture was poured into water (500 mL) and adjusted to pH=8.0 by NaOH (1M) and extracted with DCM (500 mL, 300 mL). The combined organic phase was washed with brine (300 mL) and dried over Na₂SO₄, filtered and concentrated to give the residue, which was purified by column chromatography (SiO₂, PE:EA=100/1, 0/1) to give the product (30 g, 91% yield). MS: M/e 368 (M+1)⁺.

### Step 6: (1r, 3r)-3-(3-aminophenyl)-3-((4-methyl-4H-1,2,4-triazol-3-yl)methyl)cyclobutane-1-carbonitrile

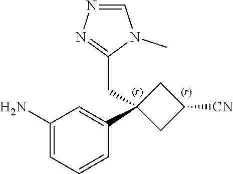 [SMILES: Cn1cnnc1C[C@]1(c2cccc(N)c2)C[C@H](C#N)C1]

To a solution of tert-butyl (3-((1r, 3r)-3-cyano-1-((4-methyl-4H-1,2,4-triazol-3-yl)methyl)cyclobutyl)phenyl)carbamate (30 g, 81.6 mmol) in DCM (600 ml) was added TMSI (32.7 g, 163.3 mmol) at 0° C. The mixture was stirred for 30 min at 0° C. The mixture was adjusted to pH=8.0 by sat NaHCO₃ and extracted with DCM (800 mL, 500 mL). The combined organic phase was washed with brine (200 mL) and dried over Na₂SO₄, filtered and concentrated to give the residue, which was purified by Prep-HPLC (column=Xtimate C18 10 u 250 mm*80 mm); mobile phase=water (NH₄HCO₃)-ACN, B %=0%-40%; 25 min) to give the product (16 g, 73% yield). MS: M/e 268 (M+1)⁺. ¹H NMR (400 MHz, DMSO-d₆) δ 8.20 (s, 1H) 6.87-6.91 (t, J=7.6 Hz, 1H) 6.38-6.40 (d, J=7.6 Hz, 1H) 6.15 (s, 1H) 6.04-6.06 (d, J=7.6 Hz, 1H) 5.14 (s, 2H) 3.18 (s, 3H) 2.67-2.79 (m, 7H).

### Compound INT4: (1s, 3s)-3-(3-aminophenyl)-3-((4-methyl-4H-1,2,4-triazol-3-yl)methyl)cyclobutane-1-carbonitrile

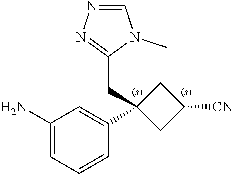 [SMILES: Cn1cnnc1C[C@]1(c2cccc(N)c2)C[C@@H](C#N)C1]

### Step 1: 2-(1-(3-((tert-butoxycarbonyl)amino)phenyl)-3-cyanocyclobutyl)acetic acid

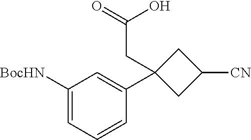 [SMILES: CC(C)(C)OC(=O)Nc1cccc(C2(CC(=O)O)CC(C#N)C2)c1]

To a solution of methyl 2-(1-(3-((tert-butoxycarbonyl)amino)phenyl)-3-cyanocyclobutyl)acetate (80 g, 232.3 mmol) in THF/MeOH/H₂O (800 ml, 1:1:1) was added LiOH·H₂O (29.2 g, 696.2 mmol) at 20° C. The mixture was stirred for 12 hrs at 20° C. under N₂. The mixture was adjusted to pH=6.0 by HCl (1 M) and extracted with EtOAc (1.5 L, 1.0 L). The combined organic phase was washed with brine (500 mL), dried over Na₂SO₄, filtered and concentrated to give the residue, which was purified by column chromatography (SiO₂, PE:EA=100/1, 0/1) to give the product (70 g, 84% yield). MS: M/e 329 (M−1)⁻.

### Step 2: tert-butyl (3-(3-cyano-1-((5-mercapto-4-methyl-4H-1,2,4-triazol-3-yl)methyl)cyclobutyl)phenyl)carbamate

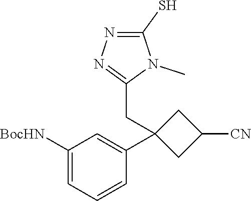 [SMILES: Cn1c(S)nnc1CC1(c2cccc(NC(=O)OC(C)(C)C)c2)CC(C#N)C1]

To a solution of 2-(1-(3-((tert-butoxycarbonyl)amino)phenyl)-3-cyanocyclobutyl)acetic acid (35 g*2, 106 mmol) in DMF (350 mL) was DIEA (15.1 g, 116.5 mmol), HATU (52.4 g, 137.7 mmol) and N-methylhydrazinecarbothioamide (16.7 g, 159.0 mmol) at 20° C. The mixture was stirred for 13 hrs at 25° C. and then added NaOH (222.5 mL, 222.5 mmol, 1M). The mixture was stirred for 12 hrs at 60° C. The mixture was poured into water (500 mL) and adjusted to pH=6.0 by HCl (1M) and extracted with EtOAc (1.0 L, 500 mL). The combined organic phase was washed with brine (300*2 mL) and dried over Na₂SO₄, filtered and concentrated to give the residue, which was purified by column chromatography (SiO₂, PE:EA=100/1, 0/1) to give the product (80 g, 81% yield). MS: M/e 400 (M+1)⁺.

### Step 3: tert-butyl (3-(3-cyano-1-((4-methyl-4H-1,2,4-triazol-3-yl)methyl)cyclobutyl)phenyl)carbamate

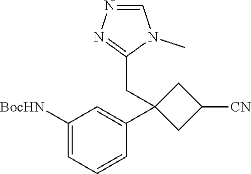 [SMILES: Cn1cnnc1CC1(c2cccc(NC(=O)OC(C)(C)C)c2)CC(C#N)C1]

To a solution of tert-butyl (3-(3-cyano-1-((5-mercapto-4-methyl-4H-1,2,4-triazol-3-yl)methyl)cyclobutyl)phenyl)carbamate (40 g, 100 mmol) in DCM (800 ml) was added AcOH (80 ml) and H₂O₂(45.4 g, 400 mmol, 30%) at 0° C. The mixture was stirred for 6 hrs at 20° C. The mixture was poured into water (500 mL) and adjusted to pH=8.0 by NaOH (1M) and extracted with DCM (800 mL, 500 mL). The combined organic phase was washed with brine (300 mL) and dried over Na₂SO₄, filtered and concentrated to give the residue, which was purified by column chromatography (SiO₂, PE:EA=100/1, 0/1) to give the product (60 g, 82% yield). MS: M/e 368 (M+1)⁺.

### Step 6: (1s, 3s)-3-(3-aminophenyl)-3-((4-methyl-4H-1,2,4-triazol-3-yl)methyl)cyclobutane-1-carbonitrile

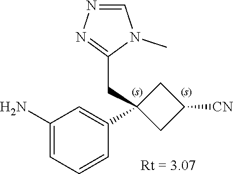 [SMILES: Cn1cnnc1C[C@]1(c2cccc(N)c2)C[C@@H](C#N)C1]

To a solution of tert-butyl (3-(3-cyano-1-((4-methyl-4H-1,2,4-triazol-3-yl)methyl)cyclobutyl)phenyl)carbamate (20 g*3, 54.4 mmol) in DCM (400 ml) was added TMSI (21.8 g, 108.8 mmol) at 0° C. The mixture was stirred for 30 min at 0° C. The mixture was adjusted to pH=8.0 by sat NaHCO₃ and extracted with DCM (1500 mL, 1000 mL). The combined organic phase was washed with brine (500 mL) and dried over Na₂SO₄, filtered and concentrated to give the residue, which was purified by Prep-HPLC (column=Welch Xtimate C18 250*70 mm #10 um); mobile phase=water (NH₄HCO₃)-ACN, B %=0%-30%; 20 min) to give the product (32 g, 73%). The product was purified by SFC (column=DAICEL CHIRALPAK IC (250 mm*50 mm, 10 um)); mobile phase=0.1% NH₃H₂O ETOH, B %=60%-60%; 5 min) to give (1s, 3s)-3-(3-aminophenyl)-3-((4-methyl-4H-1,2,4-triazol-3-yl)methyl)cyclobutane-1-carbonitrile (P1: 14.5 g, 45%); ¹HNMR (400 MHz, DMSO-d₆) δ=8.17 (s, 1H), 6.86-6.90 (t, J=7.6 Hz, 1H) 6.38-6.40 (d, J=7.6 Hz, 1H) 6.02 (s, 1H) 5.91-5.93 (d, J=7.6 Hz, 1H) 5.01 (s, 2H), 3.17-3.18 (m, 1H), 3.11 (s, 2H) 2.90-2.95 (m, 2H) 2.68 (s, 3H) 2.43-2.46 (m, 2H);

### Compound INT5: 3-((1s, 3s)-3-methyl-1-(4-methyl-4H-1,2,4-triazol-3-yl)cyclobutyl)aniline

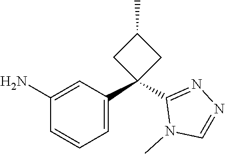 [SMILES: Cn1cnnc1[C@]1(c2cccc(N)c2)C[C@H](C)C1]

### Step 1: methyl 1-(3-bromophenyl)-3-methylcyclobutane-1-carboxylate

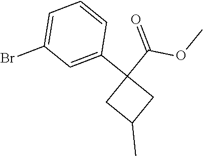 [SMILES: COC(=O)C1(c2cccc(Br)c2)CC(C)C1]

To a solution of methyl 2-(3-bromophenyl)acetate (100 g, 436 mmol) in DMF (2000 mL) was added 1,3-dibromo-2-methylpropane (94.2 g, 436 mmol) and NaH (38.4 g, 960 mmol, 60%) at 0° C. under N₂. The mixture was stirred for 12 hrs at 25° C. under N₂. The mixture was poured into sat.NH₄Cl aqueous (2.0 L) and extracted with EtOAc (1000 mL, 500 mL). The combined organic phase was washed with brine (2.0 L*2), dried over Na₂SO₄, filtered and concentrated to give the residue, which was purified by column chromatography (SiO₂, PE:EA=100/1, 0/1) to give the product (80 g, 65% yield). MS: M/e 283 (M+1)⁺.

### Step 2: 1-(3-bromophenyl)-3-methylcyclobutane-1-carbohydrazide

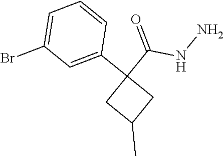 [SMILES: CC1CC(C(=O)NN)(c2cccc(Br)c2)C1]

To a solution of methyl 1-(3-bromophenyl)-3-methylcyclobutane-1-carboxylate (80 g, 282 mmol) in EtOH (800 mL) was added NH₂NH₂H₂O (70.7 g, 1.4 mol) at 25° C. The mixture was stirred for 48 hrs at 80° C. under N₂. The mixture was poured into water (1000 mL) and extracted with EtOAc (1000 mL, 500 mL). The combined organic phase was washed with brine (300 mL), dried over Na₂SO₄, filtered and concentrated to give the residue, which was purified by column chromatography (SiO₂, PE:EA=100/1, 0/1) to give the product (60 g, 75% yield). MS: M/e 283 (M+1)⁺.

### Step 3: 5-(1-(3-bromophenyl)-3-methylcyclobutyl)-4-methyl-4H-1,2,4-triazole-3-thiol

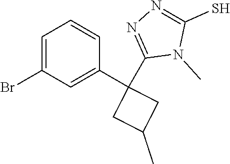 [SMILES: CC1CC(c2cccc(Br)c2)(c2nnc(S)n2C)C1]

To a solution of 1-(3-bromophenyl)-3-methylcyclobutane-1-carbohydrazide (60 g, 212 mmol) in THF (600 mL) was added isothiocyanatomethane (46.5 g, 635 mmol) at 20° C. under N₂. The mixture was stirred for 1 hr at 65° C. under N₂ and then added KOH (594 g, 1.1 mol, 10%) in H₂O at 20° C. The mixture was heated up to 65° C. and stirred for 12 hrs at 65° C. under N₂. The mixture was poured into water (600 mL) and adjusted to pH=5.0 by HCl (1 M) and extracted with EtOAc (800 mL, 500 mL). The combined organic phase was washed with brine (500 mL), dried over Na₂SO₄, filtered and concentrated to give the residue, which was purified by column chromatograghy (SiO₂, PE:EA=100/1, 0/1) to give the product (50 g, 70% yield). MS: M/e 338 (M+1)⁺

### Step 4: 3-(1-(3-bromophenyl)-3-methylcyclobutyl)-4-methyl-4H-1,2,4-triazole

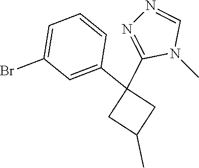 [SMILES: CC1CC(c2cccc(Br)c2)(c2nncn2C)C1]

To a solution of 5-(1-(3-bromophenyl)-3-methylcyclobutyl)-4-methyl-4H-1,2,4-triazole-3-thiol (50 g, 148 mmol) in DCM (500 mL) was added H₂O₂(50 g, 443 mmol, 30%) and AcOH (50 mL) at 0° C. The mixture was stirred for 3 hrs at 25° C. The mixture was poured into water (500 mL) and adjusted to pH=8.0 by NaOH (2M) and extracted with DCM (1000 mL, 500 mL). The combined organic phase was washed with Na₂SO₃ (500 mL) and dried over Na₂SO₄, filtered and concentrated to give the residue, which was purified by column chromatography (SiO₂, PE:EA=100/1, 0/1) to give the product (35 g, 78% yield). MS: M/e 306 (M+1)⁺.

### Step 5: 3-((1s, 3s)-1-(3-bromophenyl)-3-methylcyclobutyl)-4-methyl-4H-1,2,4-triazole

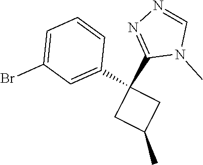 [SMILES: Cn1cnnc1[C@]1(c2cccc(Br)c2)C[C@H](C)C1]

Compound of 3-(1-(3-bromophenyl)-3-methylcyclobutyl)-4-methyl-4H-1,2,4-triazole (35 g, 114 mmol) was purified by pre-SFC-2 (DAICEL CHIRALPAK AD (250 mm*30 mm, 10 um, 0.1% NH₃H₂O ETOH) to give the product (13 g, 37% yield). MS: M/e 306 (M+1)⁺; ¹H NMR (400 MHz, DMSO-d₆) 8.31 (m, 1H) δ 7.45-7.48 (m, 2H) 7.34-7.35 (m, 2H) 3.17 (s, 3H) 2.79-2.82 (m, 2H) 2.47-2.52 (m, 3H) 1.04-1.07 (m, 3H) ppm.

### Step 6: tert-butyl (3-((1s, 3s)-3-methyl-1-(4-methyl-4H-1,2,4-triazol-3-yl)cyclobutyl)phenyl)carbamate

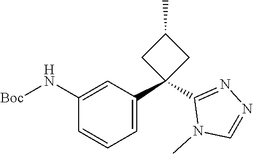 [SMILES: Cn1cnnc1[C@]1(c2cccc(NC(=O)OC(C)(C)C)c2)C[C@H](C)C1]

To a solution of 3-((1s, 3s)-1-(3-bromophenyl)-3-methylcyclobutyl)-4-methyl-4H-1,2,4-triazole (4 g, 13.07 mmol), tert-butyl carbamate (1.5 g, 12.82 mmol) in Dioxane (50 mL) were added Pd(OAc)₂ (300 mg, 1.34 mmol) and Xantphos (1.5 g, 2.59 mmol), Cs₂CO₃ (8.5 g, 26.07 mmol) under N₂. The reaction mixture was stirred for 12 hrs at 110° C. After completed, the mixture was filtered and the filate was collected and concentrated under vacuum. The residue was purified by purified by column chromatography on silica gel eluting with methanol in dichloromethane (10%) to afford the title compound (3.7 g, 83%). MS: M/e 343. (M+H)⁺.

### Step 7: 3-((1s, 3s)-3-methyl-1-(4-methyl-4H-1,2,4-triazol-3-yl)cyclobutyl)aniline

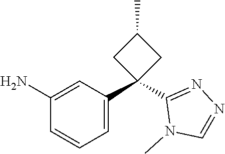 [SMILES: Cn1cnnc1[C@]1(c2cccc(N)c2)C[C@H](C)C1]

The tert-butyl (3-((1s, 3s)-3-methyl-1-(4-methyl-4H-1,2,4-triazol-3-yl)cyclobutyl)phenyl)carbamate (3.7 g, 10.82 mmol) was dissolved in HCl (60 mL, 4 M in dioxane). The reaction mixture was stirred for 5 hrs at 25° C. After completed, the solvent was concentrated in vacuo. The residue was diluted with DCM (50 mL) and quenched with aq NaHCO₃ (30 mL) and extracted with DCM/MeOH (10/1, 3×80 mL), dried over Na₂SO₄ and concentrated under vacuum. The residue was purified by column chromatography on silica gel eluting with methanol in dichloromethane (10%) to afford the title compound (1.9 g, 73%). ¹H NMR (400 MHz, CD₃OD) δ 8.26 (s, 1H), 7.09 (t, J=8.4 Hz, 1H), 6.69-6.65 (m, 2H), 6.61 (d, J=8.0 Hz, 1H), 3.24 (s, 3H), 2.89-2.79 (m, 2H), 2.69-2.56 (m, 1H), 2.53-2.44 (m, 2H), 1.12 (d, J=6.4 Hz, 3H) ppm. MS: M/e 243 (M+H)⁺.

### Compound INT6: 2-((1s, 3s)-3-(3-aminophenyl)-3-(4-methyl-4H-1,2,4-triazol-3-yl)cyclobutyl)acetonitrile

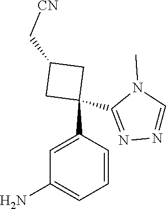 [SMILES: Cn1cnnc1[C@]1(c2cccc(N)c2)C[C@H](CC#N)C1]

### Step 1: 1-(3-bromophenyl)-3,3-dimethoxycyclobutanecarbonitrile

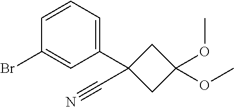 [SMILES: COC1(OC)CC(C#N)(c2cccc(Br)c2)C1]

To a solution of NaH (9.0 g, 224 mmol, 60%) in DMF (200 mL) was added 2-(3-bromophenyl)acetonitrile (20 g, 102 mmol) at 0° C. under N₂. The mixture was stirred for 10 min and then added 1,3-dibromo-2,2-dimethoxypropane (29.4 g, 112 mmol) at 0° C. under N₂. The mixture was stirred for 12 hrs at 60° C. under N₂. The mixture was poured into sat.NH₄Cl aqueous (200 mL) and extracted with EtOAc (200 mL, 100 mL). The combined organic phase was washed with brine (100 mL), dried over Na₂SO₄, filtered and concentrated to give the residue, which was purified by column chromatography (SiO₂, PE:EA=100/1, 0/1) to give the product (14 g, 48% yield). ¹H NMR: (400 MHz, CDCl₃-d) δ 7.55-7.56 (t, J=2 Hz, 1H) 7.39-7.41 (d, J=8 Hz, 1H) 7.33-7.35 (d, J=8 Hz, 1H) 7.21-7.23 (m, 1H) 3.21 (s, 3H) 3.11 (s, 3H) 3.01-3.05 (m, 2H) 2.61-2.65 (m, 2H) ppm;

### Step 2: 1-(3-bromophenyl)-3,3-dimethoxycyclobutanecarboxylic acid

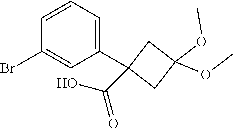 [SMILES: COC1(OC)CC(C(=O)O)(c2cccc(Br)c2)C1]

To a solution of 1-(3-bromophenyl)-3,3-dimethoxycyclobutanecarbonitrile (14 g, 47 mmol) in EtOH (60 mL) was added NaOH (56.7 g, 142 mmol, 10%) and stirred for 15 hrs at 85° C. under N₂. The mixture was poured into water (20 mL) and adjusted to pH=3.0 by HCl (2 M) and extracted with EtOAc (100 mL, 50 mL). The combined organic phase was washed with brine (20 mL), dried over Na₂SO₄, filtered and concentrated to give the residue, which was purified by column chromatograghy (SiO₂, PE:EA=100/1, 0/1) to give the product (9.0 g, 60% yield). ¹H NMR: (400 MHz, CDCl₃-d) δ 7.46 (s, 1H) 7.39-7.42 (m, 1H) 7.21-7.26 (m, 2H) 3.20 (s, 3H) 3.11-3.16 (m, 5H) 2.54-2.57 (m, 2H); MS: M/e 313 (M−1)⁻

### Step 3: 1-(3-bromophenyl)-3,3-dimethoxycyclobutanecarbohydrazide

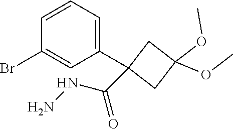 [SMILES: COC1(OC)CC(C(=O)NN)(c2cccc(Br)c2)C1]

To a solution of 1-(3-bromophenyl)-3,3-dimethoxycyclobutanecarboxylic acid (9.0 g, 28.6 mmol) in DCM (100 ml) was cooled down to 0° C. and added Et₃N (5.8 g, 57 mmol) and isobutyl carbonochloridate (5.8 g, 42.8 mmol) by dropwise at 0° C. under N₂ The mixture was stirred for 40 min at 0° C. and then added NH₂NH₂H₂O (5.7 g, 114 mmol) by dropwise at −30° C. The mixture was stirred for 3 hrs at 20° C. under N₂. The mixture was poured into water (100 mL) and extracted with EtOAc (100 mL, 50 mL). The combined organic phase was washed with brine (50 mL), dried over Na₂SO₄, filtered and concentrated to give the residue, which was purified by column chromatograghy (SiO₂, PE:EA=100/1, 0/1) to give the product (6.0 g, 68% yield). ¹H NMR: (400 MHz, CDCl₃-d) δ 7.40-7.43 (m, 2H) 7.21-7.23 (m, 2H) 6.87 (s, 1H) 3.69 (s, 2H) 3.21 (s, 3H) 3.10 (s, 3H) 3.05-3.08 (m, 2H) 2.61-2.64 (m, 2H) ppm.

### Step 4: 5-(1-(3-bromophenyl)-3,3-dimethoxycyclobutyl)-4-methyl-4H-1,2,4-triazole-3-thiol

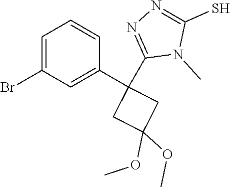 [SMILES: COC1(OC)CC(c2cccc(Br)c2)(c2nnc(S)n2C)C1]

To a solution of 1-(3-bromophenyl)-3,3-dimethoxycyclobutanecarbohydrazide (6.0 g, 19.2 mmol) in THF (120 mL) was added isothiocyanatomethane (4.2 g, 57.4 mmol) at 20° C. under N₂. The mixture was stirred for 1 hr at 65° C. under N₂ and then added KOH (5.4 g, 95.7 mmol) in H₂O (12 mL) at 20° C. The mixture was heated up to 65° C. and stirred for 12 hrs at 65° C. under N₂. The mixture was poured into water (50 mL) and adjusted to pH=5.0 by HCl (1 M) and extracted with EtOAc (100 mL, 50 mL). The combined organic phase was washed with brine (50 mL), dried over Na₂SO₄, filtered and concentrated to give the residue, which was purified by column chromatograghy (SiO₂, PE:EA=100/1, 0/1) to give the product (5.5 g, 79% yield); ¹H NMR: (400 MHz, CDCl₃-d) 11.20 (s, 1H) δ 7.42-7.44 (m, 2H) 7.21-7.25 (t, J=8 Hz, 1H) 7.12-7.14 (d, J=8 Hz, 1H) 3.18-3.22 (m, 11H) 2.81-2.84 (m, 2H) ppm.

### Step 5: 3-(1-(3-bromophenyl)-3,3-dimethoxycyclobutyl)-4-methyl-4H-1,2,4-triazole

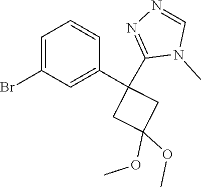 [SMILES: COC1(OC)CC(c2cccc(Br)c2)(c2nncn2C)C1]

To a solution of 5-(1-(3-bromophenyl)-3,3-dimethoxycyclobutyl)-4-methyl-4H-1,2,4-triazole-3-thiol (2.0 g, 5.2 mmol) in DCM (40 mL) was added H₂O₂(2.9 g, 26 mmol, 30%) and AcOH (4 mL) at 15° C. The mixture was stirred for 3 hrs at 15° C. The mixture was poured into water (10 mL) and adjusted to pH=8.0 by NaOH (1M) and extracted with DCM (20 mL, 10 mL). The combined organic phase was washed with Na₂SO₃ (10 mL) and brine(10 mL) dried over Na₂SO₄, filtered and concentrated to give the residue, which was purified by column chromatograghy (SiO₂, PE:EA=100/1, 0/1) to give the product (1.4 g, 77% yield);

### Step 6: 3-(3-bromophenyl)-3-(4-methyl-4H-1,2,4-triazol-3-yl)cyclobutanone

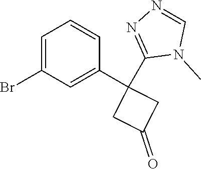 [SMILES: Cn1cnnc1C1(c2cccc(Br)c2)CC(=O)C1]

To a solution of 3-(1-(3-bromophenyl)-3,3-dimethoxycyclobutyl)-4-methyl-4H-1,2,4-triazole (1.4 g, 4.0 mmol) in acetone (20 mL) was added HCl (10 ml, 6 M) at 15° C. The mixture was stirred for 12 hrs at 15° C. The mixture was poured into water (10 mL) and adjusted to pH=8.0 by NaOH (2 M) and extracted with EtOAc (50 mL, 20 mL). The combined organic phase was washed with brine (30 mL), dried over Na₂SO₄, filtered and concentrated to give the residue, which was purified by column chromatograghy (SiO₂, PE:EA=100/1, 0/1) to give the product (1.0 g, 83% yield);

### Step 7: 2-(3-(3-bromophenyl)-3-(4-methyl-4H-1,2,4-triazol-3-yl)cyclobutylidene)acetonitrile

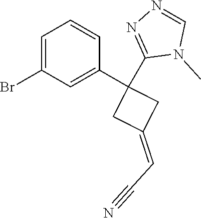 [SMILES: Cn1cnnc1C1(c2cccc(Br)c2)CC(=CC#N)C1]

To a solution of 3-(3-bromophenyl)-3-(4-methyl-4H-1,2,4-triazol-3-yl)cyclobutanone (800 mg, 2.6 mmol) in THF (16 mL) was added diethyl (cyanomethyl)phosphonate (509 mg, 2.87 mmol) and t-BuOK (322 mg, 2.87 mmol) at 0° C. and stirred for 6 hrs at 15° C. under N₂. The mixture was poured into sat. NH₄Cl aqueous (10 ml) and extracted with EtOAc (20 mL, 10 mL). The combined organic phase was washed with brine (10 mL), dried over Na₂SO₄, filtered and concentrated to give the residue, which was purified by column chromatograghy (SiO₂, PE:EA=100/1, 0/1) to give the product (600 mg, 70% yield). MS: M/e 329 (M+1)⁺

### Step 8: 2-(3-(3-bromophenyl)-3-(4-methyl-4H-1,2,4-triazol-3-yl)cyclobutyl)acetonitrile

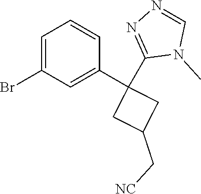 [SMILES: Cn1cnnc1C1(c2cccc(Br)c2)CC(CC#N)C1]

To a solution of 2-(3-(3-bromophenyl)-3-(4-methyl-4H-1,2,4-triazol-3-yl)cyclobutylidene)acetonitrile (2.6 g, 8.0 mmol) in EtOH (25 mL) and iPrOH (25 mL) was added NaBH₄ (896 mg, 23.7 mmol) at 15° C. The mixture was stirred for 6 hrs at 60° C. The mixture was poured into sat. NH₄Cl aqueous (50 ml) and extracted with EtOAc (50 mL, 30 mL). The combined organic phase was washed with brine (20 mL), dried over Na₂SO₄, filtered and concentrated to give the residue, which was purified by column chromatography (SiO₂, EA: MeOH=100/1, 1/1) to give the product (2.0 g, 75% yield). MS: M/e 331 (M+1)⁺;

2-(3-(3-bromophenyl)-3-(4-methyl-4H-1,2,4-triazol-3-yl)cyclobutyl)acetonitrile (360 mg) was purified by SFC (column=ChiralPak IH, 250*30 mm, 10 um; mobile phase=Neu-IPA, B %=45%-45%; 4 min) to give two isomers P1 and P2;

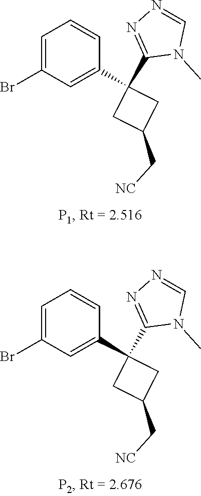 [SMILES: Cn1cnnc1[C@]1(c2cccc(Br)c2)C[C@H](CC#N)C1 ; Cn1cnnc1[C@]1(c2cccc(Br)c2)C[C@@H](CC#N)C1]

2-((1s, 3s)-3-(3-bromophenyl)-3-(4-methyl-4H-1,2,4-triazol-3-yl)cyclobutyl)acetonitrile (P1, Rt=2.516, 103 mg, 28.7%); ¹H NMR (400 MHz, DMSO-d₆) δ=8.32 (s, 1H), 7.47-7.50 (m, 2H), 7.34-7.36 (m, 2H), 3.18 (s, 3H), 2.82-2.88 (m, 2H), 2.71-2.73 (m, 5H) ppm; MS: M/e 331 (M+1)⁺.

2-((1r, 3r)-3-(3-bromophenyl)-3-(4-methyl-4H-1,2,4-triazol-3-yl)cyclobutyl)acetonitrile (P2, Rt=2.676; 125 mg, 34.8%); ¹H NMR (400 MHz, DMSO-d₆) δ=8.41 (s, 1H), 7.45-7.47 (m, 1H), 7.32-7.36 (m, 2H), 7.16-7.20 (m, 1H), 3.20 (s, 3H), 3.06-3.11 (m, 2H), 2.71-2.73 (m, 2H). 2.40-2.50 (m, 3H) ppm. MS: M/e 331 (M+1)⁺.

### Step 9: 2-(3-(3-aminophenyl)-3-(4-methyl-4H-1,2,4-triazol-3-yl)cyclobutyl)acetonitrile

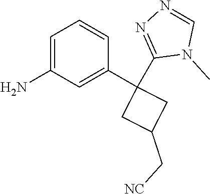 [SMILES: Cn1cnnc1C1(c2cccc(N)c2)CC(CC#N)C1]

To a solution of 2-(3-(3-bromophenyl)-3-(4-methyl-4H-1,2,4-triazol-3-yl)cyclobutyl)acetonitrile (1.0 g, 3 mmol) in sulfolane (10 mL) was added Dimethyl Ethylene Diamine (DMEDA, 53 mg, 604 umol), CuI (115 mg, 604 umol) and NH₃H₂O (20 ml) at 25° C. under N₂. The mixture was heated up to 100° C. and stirred for 16 hrs at 100° C. in a seal. The mixture was filtered to give the filtrate which was purified by prep-HPLC (column=Welch Xtimate C18 250*70 mm #10 um); mobile phase=water (NH₄HCO₃)-ACN, B %=5%-35%; 20 min) to give the product (480 mg, 60%), MS: M/e 268 (M+1)⁺.

2-(3-(3-aminophenyl)-3-(4-methyl-4H-1,2,4-triazol-3-yl)cyclobutyl)acetonitrile was purified by SFC (column=DAICEL CHIRALPAK AD (250 mm*30 mm, 10 um); mobile phase=0.1% NH₃H₂O ETOH, B %=24%-24%; 9 min) to give two isomers P1 and P2.

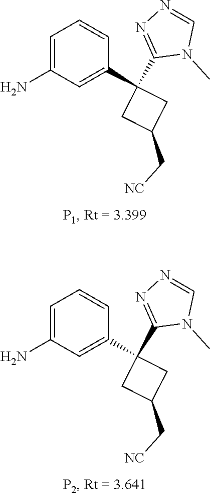 [SMILES: Cn1cnnc1[C@]1(c2cccc(N)c2)C[C@@H](CC#N)C1 ; Cn1cnnc1[C@]1(c2cccc(N)c2)C[C@H](CC#N)C1]

2-((1r, 3r)-3-(3-aminophenyl)-3-(4-methyl-4H-1,2,4-triazol-3-yl)cyclobutyl)acetonitrile (P1, Rt=3.399, 122 mg, 30%); ¹H NMR (400 MHz, DMSO-d₆) δ=8.36 (s, 1H), 6.97-7.01 (t, J=8.0 Hz, 1H) 6.37-6.42 (m, 2H) 6.26 (s, 1H) 5.09 (s, 2H) 3.19 (s, 3H), 2.95-3.00 (m, 2H), 2.67-2.69 (m, 2H) 2.51-2.52 (m, 1H) 2.32-2.35 (m, 2H) ppm. MS: M/e 268 (M+1)⁺.

2-((1s, 3s)-3-(3-aminophenyl)-3-(4-methyl-4H-1,2,4-triazol-3-yl)cyclobutyl)acetonitrile (P2, Rt=3.641, 105 mg, 25%): ¹H NMR (400 MHz, DMSO-d₆) δ=8.28 (s, 1H), 6.96-7.01 (t, J=7.6 Hz, 1H), 6.43-6.50 (m, 3H) 5.09 (s, 2H) 3.16-3.17 (m, 4H), 2.65-2.73 (m, 5H), 2.51-2.52 (m, 1H) ppm; MS: M/e 268 (M+1)⁺.

### Compound INT7:3-((1s, 3s)-3-methyl-1-(4-(methyl-d3)-4H-1,2,4-triazol-3-yl)cyclobutyl)aniline

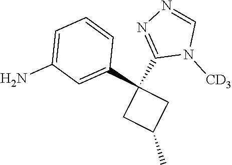 [SMILES: [2H]C([2H])([2H])n1cnnc1[C@]1(c2cccc(N)c2)C[C@H](C)C1]

### Step 1: (E)-N′-(1-(3-bromophenyl)-3-methylcyclobutane-1-carbonyl)-N,N-dimethylformohydrazonamide

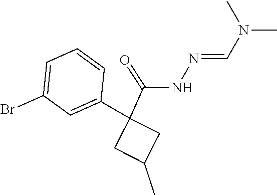 [SMILES: CC1CC(C(=O)N/N=C/N(C)C)(c2cccc(Br)c2)C1]

To a solution of 1-(3-bromophenyl)-3-methyl-cyclobutanecarbohydrazide (4 g, 14.13 mmol, 1 eq) in DCM (80 mL) was added DMFDMA (8.42 g, 70.63 mmol, 9.38 mL, 5 eq) at 20° C. The mixture was stirred at 50° C. for 16 hr. The mixture was filtered and concentrated under reduced pressure to give a residue. The residue was purified by column chromatography (SiO2, PE/EA=10/1 to DCM:MEOH=10:1) to give Compound 1-(3-bromophenyl)-N—[(E)-dimethylaminomethyleneamino]-3-methyl-cyclobutanecarboxamide (4.6 g, 13.60 mmol) was obtained as a yellow solid. MS [M+41]340.

### Step 2: 3-((1s, 3s)-1-(3-bromophenyl)-3-methylcyclobutyl)-4-(methyl-d3)-4H-1,2,4-triazole

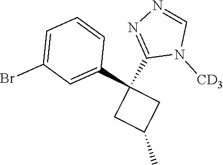 [SMILES: [2H]C([2H])([2H])n1cnnc1[C@]1(c2cccc(Br)c2)C[C@H](C)C1]

To a solution of 1-(3-bromophenyl)-N—[(E)-dimethylaminomethyleneamino]-3-methyl-cyclobutanecarboxamide (5 g, 14.78 mmol, 1 eq) in THF (100 mL) was added trideuteriomethanamine; hydrochloride (4.99 g, 73.91 mmol, 5 eq) and CH3COOH (887.68 mg, 14.78 mmol, 846.21 L, 1 eq) and TEA (7.48 g, 73.91 mmol, 10.29 mL, 5 eq). The mixture was stirred at 90° C. for 48 hr. The reaction mixture was diluted with H2O 100 mL and extracted with EA 150 mL (50 mL*3). dried over [Na2SO4], filtered and concentrated under reduced pressure to give a residue. The residue was purified by prep-HPLC (column: Welch Xtimate C18 180*70 mm #10 um; mobile phase: [H2O (10 mM NH4HCO3)-ACN]; gradient: 17%-47% B over 20.0 min). The residue was purified by prep-HPLC (column: Welch Xtimate C18 180*70 mm #10 um; mobile phase: [H2O (10 mM NH4HCO3)-ACN]; gradient: 17%-47% B over 20.0 min) to give Compound 3-[1-(3-bromophenyl)-3-methyl-cyclobutyl]-4-(trideuteriomethyl)-1,2,4-triazole (1.1 g, 3.56 mmol, 24.06% yield) was obtained as a brown solid. MS [M+1] 311; ¹H NMR (400 MHz, CHLOROFORM-d) δ=7.97 (s, 1H), 7.53 (s, 1H), 7.44-7.31 (m, 1H), 7.26-7.06 (m, 2H), 2.88-2.74 (m, 2H), 2.72-2.57 (m, 3H), 1.14 (d, J=5.4 Hz, 3H).

### Step 5: 3-((1s, 3s)-3-methyl-1-(4-(methyl-d3)-4H-1,2,4-triazol-3-yl)cyclobutyl)aniline

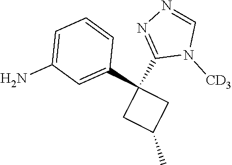 [SMILES: [2H]C([2H])([2H])n1cnnc1[C@]1(c2cccc(N)c2)C[C@H](C)C1]

To a solution of 3-[1-(3-bromophenyl)-3-methyl-cyclobutyl]-4-(trideuteriomethyl)-1,2,4-triazole (400.00 mg, 1.29 mmol, 1 eq) in SULFOLANE (3 mL) and NH3·H2O (9 mL) was added CuI (246.36 mg, 1.29 mmol, 1 eq) and DMEDA (114.03 mg, 1.29 mmol, 139.23 L, 1 eq). The mixture was stirred at 100° C. for 6 hr. The reaction mixture was extracted with DCM 30 mL (15 mL*2). dried over Na2SO4, filtered and concentrated under reduced pressure to give a residue. The residue was purified by Prep-HPLC(column: Phenomenex luna C18 100*40 mm*3 um; mobile phase: [water (0.04% HCl)-ACN]; gradient: 1%-20% B over 8.0 min). MS [M+1] 246; ¹H NMR (400 MHz, DMSO-d₆) δ=9.10 (s, 1H), 7.48-7.38 (m, 1H), 7.30 (br d, J=7.8 Hz, 1H), 7.26-7.14 (m, 2H), 2.88-2.74 (m, 2H), 2.64-2.52 (m, 3H), 1.08 (br d, J=5.8 Hz, 3H).

### Compound INT8: 5-((1s, 3s)-3-methyl-1-(4-methyl-4H-1,2,4-triazol-3-yl)cyclobutyl)pyridin-3-amine

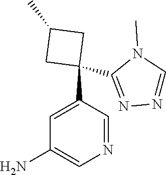 [SMILES: Cn1cnnc1[C@]1(c2cncc(N)c2)C[C@H](C)C1]

### Step 1: 1-(5-bromopyridin-3-yl)-3-methylcyclobutane-1-carbonitrile

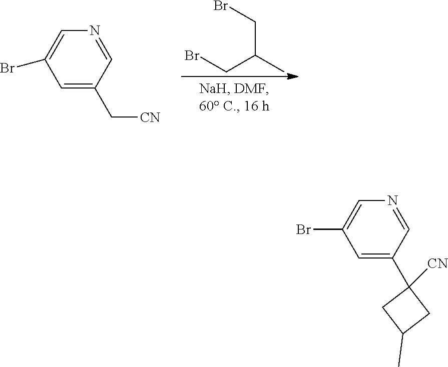

To a stirred solution of NaH (2.19 g, 86.786 mmol, 2 equiv, 95%) in DMF (300 ml) was added 2-(5-bromopyridin-3-yl)acetonitrile (9 g, 43.393 mmol, 1 equiv, 95%) dropwise at 0° C. under nitrogen atmosphere. The resulting mixture was stirred for additional 15 min at 0° C. To the above mixture was added 1,3-dibromo-2-methylpropane (14.79 g, 65.090 mmol, 1.5 equiv, 95%) at 0° C. The resulting mixture was stirred for additional 16 h at 60° C. The reaction was quenched by the addition of sat. NH₄Cl (aq.) (200 mL) at 0° C. The aqueous layer was extracted with EtOAc (2×200 mL). The resulting mixture was concentrated under reduced pressure. The residue was purified by silica gel column chromatography, eluted with PE/EA (1:1) to afford 1-(5-bromopyridin-3-yl)-3-methylcyclobutane-1-carbonitrile (9.0 g, 74.33%) as a off-white solid. LC-MS (M+H)⁺:=251.

### Step 2: 1-(5-bromopyridin-3-yl)-3-methylcyclobutane-1-carboxylic acid

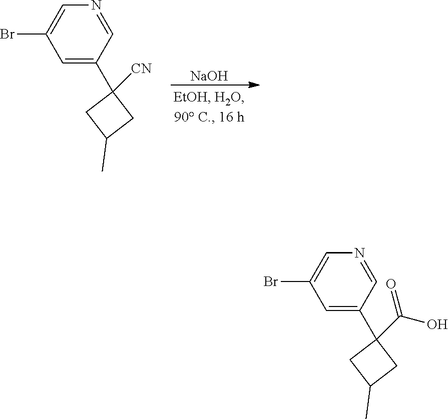

A solution of 1-(5-bromopyridin-3-yl)-3-methylcyclobutane-1-carbonitrile (9 g, 34.047 mmol, 1 equiv, 95%) and NaOH (5.73 g, 136.188 mmol, 4 equiv, 95%) in EtOH (150 ml)/H₂O (50 ml) was stirred for overnight at 90° C. under nitrogen atmosphere. The resulting mixture was concentrated under reduced pressure. The mixture was acidified to pH 2 with HCl (aq.). The resulting mixture was concentrated under reduced pressure. The residue was purified by silica gel column chromatography, eluted with CH₂Cl₂/MeOH (10:1) to afford 1-(5-bromopyridin-3-yl)-3-methylcyclobutane-1-carboxylic acid (8.2 g, 89.16%) as a off-white solid. LC-MS (M+H)⁺:=270.

### Step 3: 1-(5-bromopyridin-3-yl)-3-methylcyclobutane-1-carbohydrazide

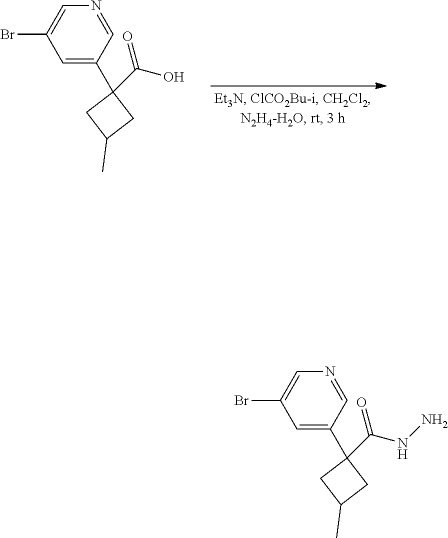 [SMILES: CC1CC(C(=O)O)(c2cncc(Br)c2)C1 ; CC1CC(C(=O)NN)(c2cncc(Br)c2)C1]

To a stirred solution of 1-(5-bromopyridin-3-yl)-3-methylcyclobutane-1-carboxylic acid (8.2 g, 28.838 mmol, 1 equiv, 95%) and Et₃N (7.07 g, 66.327 mmol, 2.3 equiv, 95%) in DCM (200 ml) was added 2-methylpropyl carbonochloridate (4.56 g, 31.722 mmol, 1.1 equiv, 95%) dropwise at 0° C. under nitrogen atmosphere. The resulting mixture was stirred for additional 1 h at room temperature. To the above mixture was added NH₂NH₂·H₂O (6.08 g, 115.352 mmol, 4 equiv, 95%) dropwise at 0° C. The resulting mixture was stirred for additional 1 h at room temperature. The resulting mixture was concentrated under reduced pressure. The residue was purified by silica gel column chromatography, eluted with CH₂Cl₂/MeOH (10:1) to afford 1-(5-bromopyridin-3-yl)-3-methylcyclobutane-1-carbohydrazide (6.42 g, 70.51%) as a off-white oil. LC-MS (M+H)⁺:=284.

### Step 4: 5-(1-(5-bromopyridin-3-yl)-3-methylcyclobutyl)-4-methyl-4H-1,2,4-triazole-3-thiol

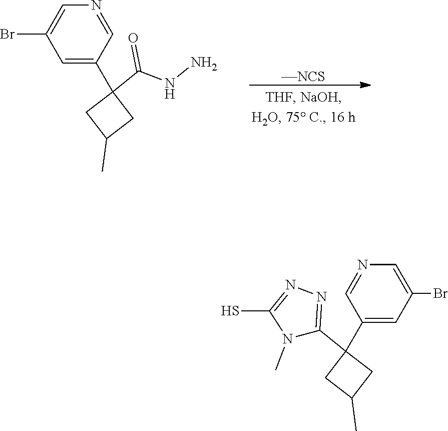

A solution of 1-(5-bromopyridin-3-yl)-3-methylcyclobutane-1-carbohydrazide (6.42 g, 21.463 mmol, 1 equiv, 95%) and isothiocyanatomethane (4.96 g, 64.389 mmol, 3 equiv, 95%) in THF (200 ml)/H₂O (100 ml) was stirred for 1 h at 65° C. under nitrogen atmosphere. To the above mixture was added NaOH (6.33 g, 150.241 mmol, 7 equiv, 95%) at room temperature. The resulting mixture was stirred for additional overnight at 75° C. The mixture was acidified to pH 2 with HCl (aq.). The resulting mixture was concentrated under reduced pressure. The residue was purified by silica gel column chromatography, eluted with CH₂Cl₂/MeOH (10:1) to afford 5-[1-(5-bromopyridin-3-yl)-3-methylcyclobutyl]-4-methyl-1,2,4-triazole-3-thiol (6.03 g, 82.81%) as a off-white oil. LC-MS (M+H)⁺: =339.

### Step 5: 3-bromo-5-(3-methyl-1-(4-methyl-4H-1,2,4-triazol-3-yl)cyclobutyl)pyridine

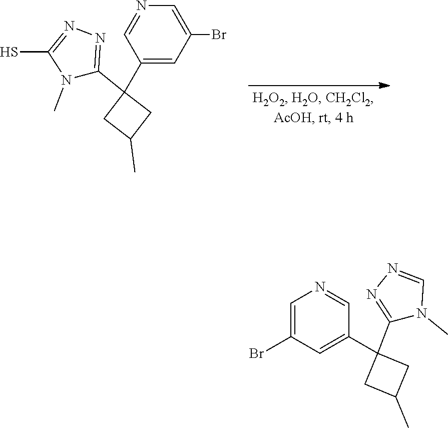 [SMILES: CC1CC(c2cncc(Br)c2)(c2nnc(S)n2C)C1 ; CC1CC(c2cncc(Br)c2)(c2nncn2C)C1]

A solution of 5-[1-(5-bromopyridin-3-yl)-3-methylcyclobutyl]-4-methyl-1,2,4-triazole-3-thiol (6.03 g, 16.885 mmol, 1 equiv, 95%) and H₂O₂ (30%) (8.23 g, 72.606 mmol, 4.3 equiv, 30%) in DCM (150 ml)/AcOH (20 ml) was stirred for 3 h at 0° C. under nitrogen atmosphere. The mixture was basified to pH 8 with NaOH. The resulting mixture was extracted with EtOAc (2×30 mL). The resulting mixture was concentrated under reduced pressure. The residue was purified by silica gel column chromatography, eluted with CH₂Cl₂/MeOH (10:1) to afford 3-bromo-5-[3-methyl-1-(4-methyl-1,2,4-triazol-3-yl)cyclobutyl]pyridine (5.0 g, 86.75%) as a off-white oil. LC-MS (M+H)⁺:=307.

### Step 6: N-(5-((1s, 3s)-3-methyl-1-(4-methyl-4H-1,2,4-triazol-3-yl)cyclobutyl)pyridin-3-yl)-1,1-diphenylmethanimine

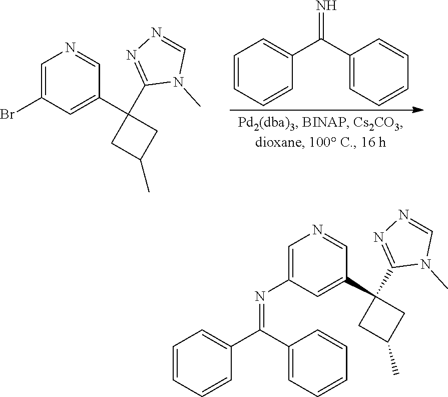

To a stirred solution of 3-bromo-5-[3-methyl-1-(4-methyl-1,2,4-triazol-3-yl)cyclobutyl]pyridine (5.0 g, 15.462 mmol, 1 equiv, 95%) and benzenemethanimine, α-phenyl-(5.90 g, 30.924 mmol, 2 equiv, 95%) in dioxane (250 ml) were added Pd₂(dba)₃ (0.75 g, 0.773 mmol, 0.05 equiv, 95%), BINAP (1.01 g, 1.546 mmol, 0.1 equiv, 95%) and Cs₂CO₃ (15.91 g, 46.386 mmol, 3 equiv, 95%) at 100° C. under nitrogen atmosphere. The crude product was purified by Chiral-Prep-HPLC with the following conditions (2 #SHIMADZU (HPLC-01)): Column, Kinetex EVO C18, 21.2*250 mm, 5 um; mobile phase, 10 mmol NH₄HCO₃+0.05% NH₃H₂O and ACN (15% ACN up to 45% in 8 min); Detector, UV product was obtained which Alpha. This resulted in N-{5-[3-methyl-1-(4-methyl-1,2,4-triazol-3-yl)cyclobutyl]pyridin-3-yl}-1,1-diphenylmethanimine (4.9 g, 69.99%) as a off-white solid. LC-MS (M+H)⁺:=408. CHIRALPAK IC-3 4.6*50 mm 3 um, Mobile phase: Hex (0.1% DEA):EtOH=50:50, Flow: 1.0 mL/min, RT=6.27 min.

### Step 7: 5-((1s, 3s)-3-methyl-1-(4-methyl-4H-1,2,4-triazol-3-yl)cyclobutyl)pyridin-3-amine

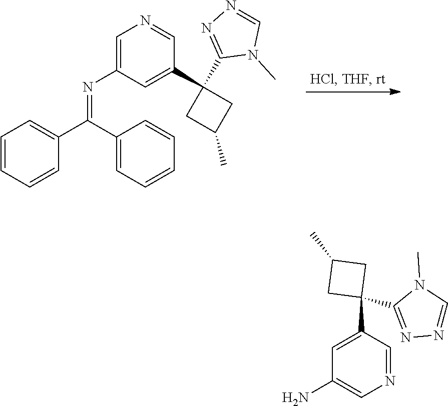 [SMILES: Cn1cnnc1[C@]1(c2cncc(N=C(c3ccccc3)c3ccccc3)c2)C[C@H](C)C1 ; Cn1cnnc1[C@]1(c2cncc(N)c2)C[C@H](C)C1]

A solution of 1,1-diphenyl-N-{5-[(1r, 3s)-3-methyl-1-(4-methyl-1,2,4-triazol-3-yl)cyclobutyl]pyridin-3-yl}methanimine (400 mg, 0.932 mmol, 1 equiv, 95%) and HCl (3399.78 mg, 2.796 mmol, 3 equiv, 3%) in THF (10 ml) was stirred for 3 h at room temperature under nitrogen atmosphere. The mixture was basified to pH 8 with NaOH. The residue was purified by reversed-phase flash chromatography with the following conditions: column, C18 silica gel; mobile phase, MeCN in Water (10 mmol/L NH4HCO3), 0% to 100% gradient in 30 min; detector, UV 254 nm. This resulted in 5-[(1r, 3s)-3-methyl-1-(4-methyl-1,2,4-triazol-3-yl)cyclobutyl]pyridin-3-amine (210 mg, 83.30%) as a off-white solid. LC-MS (M+H)⁺:=244.

### Compound Synthesis

### Compound A1: N-(3-((1s,3R)-3-methyl-1-(4-methyl-4H-1,2,4-triazol-3-yl)cyclobutyl)phenyl)-6-(((S)-3-methylpiperidin-1-yl)methyl)imidazo[1,2-a]pyridine-8-carboxamide

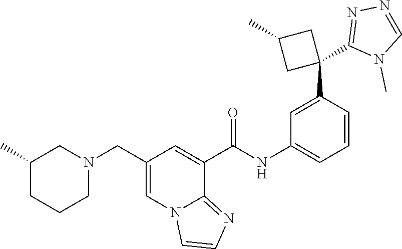 [SMILES: C[C@H]1CCCN(Cc2cc(C(=O)Nc3cccc([C@]4(c5nncn5C)C[C@@H](C)C4)c3)c3nccn3c2)C1]

### Step 1: methyl 6-vinylimidazo[1,2-a]pyridine-8-carboxylate

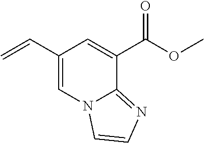

To a solution of methyl 6-bromoimidazo[1,2-a]pyridine-8-carboxylate (10 g, 39.21 mmol) in dioxane (90 mL)/H₂O (30 mL) was added potassium ethenyl(trifluoro)boranuide (13.1 g, 98.01 mmol), K₂CO₃ (16.3 g, 117.6 mmol) and Pd(dppf)Cl₂ (2.87 g, 3.92 mmol) at 15° C. The mixture was stirred for 16 hrs at 100° C. The reaction was filtered and concentrated. The crude was purified by column chromatography (SiO₂, Petroleum ether/EtOAc=5:1 to 0:1) to give the product (6.3 g, 79% yield). MS: M/e 203 (M+H)⁺; ¹H NMR (400 MHz, CDCl₃-d) 6=8.24 (s, 1H), 8.21 (d, J=1.2 Hz, 1H), 7.76 (s, 1H), 7.64 (s, 1H), 6.67 (dd, J=12.0, 16.0 Hz, 1H), 5.85 (d, J=16.0 Hz, 1H), 5.42 (d, J=12.0 Hz, 1H), 4.09 (s, 3H) ppm.

### Step 2: methyl 6-formylimidazo[1,2-a]pyridine-8-carboxylate

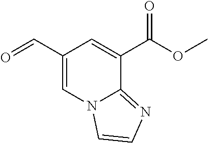

To a solution of methyl 6-vinylimidazo[1,2-a]pyridine-8-carboxylate (6.3 g, 31.1 mmol) in dioxane (125 mL)/H₂O (25 mL) was added K₂OsO₄ 2H₂O (1.15 g, 3.1 mmol), NaIO₄ (26.6 g, 124.6 mmol) in portions. The mixture was stirred for 16 hrs at 25° C. The reaction was poured into H₂O (100 mL) extracted with EA (25 mL*3). The combined organic phase was concentrated to give the crude product. The was purified by column chromatography (SiO₂, Petroleum ether/EtOAc=5:1 to 0:1) to give the product (4 g, 62% yield). MS: M/e 205 (M+H)⁺; 1H NMR (400 MHz, DMSO-d₆) δ=9.99 (s, 1H), 9.54 (d, J=1.2 Hz, 1H), 8.26 (d, J=1.2 Hz, 1H), 8.14 (d, J=1.2 Hz, 1H), 7.80 (d, J=1.2 Hz, 1H), 3.92 (s, 3H) ppm.

### Step 3: (S)-methyl 6-((3-methylpiperidin-1-yl)methyl)imidazo[1,2-a]pyridine-8-carboxylate

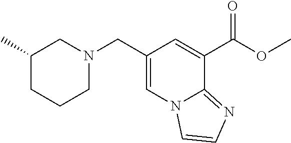

To a solution of methyl 6-formylimidazo[1,2-a]pyridine-8-carboxylate (1 g, 4.9 mmol), (S)-3-methylpiperidine hydrochloride (1.3 g, 9.8 mmol), Et₃N (495 mg, 4.9 mmol) in DCM (10 mL) was added NaBH(OAc)₃ (2.1 g, 9.8 mmol) one portion. The mixture was stirred at 25° C. for 16 hrs. The reaction was concentrated. The crude was purified by column chromatography (SiO₂, Petroleum ether/EtOAc=1:1 to 0:1) to give the product (700 mg, 49% yield). MS: M/e 288 (M+H)⁺; 1H NMR (400 MHz, DMSO-d₆) δ=8.70 (s, 1H), 8.03 (s, 1H), 7.82 (s, 1H), 7.63 (s, 1H), 3.90 (s, 3H) 3.51-3.43 (m, 2H), 1.96-1.87 (m, 1H), 1.96-1.86 (m, 1H), 1.71-1.40 (m, 6H), 0.92-0.78 (m, 4H) ppm.

### Step 4: (S)-6-((3-methylpiperidin-1-yl)methyl)imidazo[1,2-a]pyridine-8-carboxamide

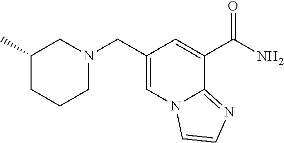 [SMILES: C[C@H]1CCCN(Cc2cc(C(N)=O)c3nccn3c2)C1]

To a solution of (S)-ethyl 6-((3-methylpiperidin-1-yl)methyl)imidazo[1,2-a]pyridine-8-carboxylate (1.3 g, 4.3 mmol) in NH₃/MeOH (15 mL. 3 M) was heated up to 70° C. in a seal tube. The Mixture was stirred for 15 hrs at 70° C. The mixture was concentrated to give the crude product (650 mg, crude), which was used to the next step without purification. MS: M/e 273 (M+H)⁺.

### Step 5: (S)-6-((3-methylpiperidin-1-yl)methyl)imidazo[1,2-a]pyridine-8-carboxylic acid

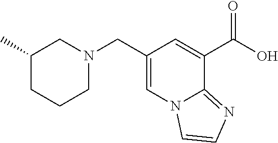 [SMILES: C[C@H]1CCCN(Cc2cc(C(=O)O)c3nccn3c2)C1]

LiOH·H₂O solution (105 mg, 2.5 mmol, in 1 mL of water) was added to a solution of methyl (S)-6-((3-methylpiperidin-1-yl)methyl)imidazo[1,2-a]pyridine-8-carboxylate (150 mg, 0.52 mmol) in MeOH (5 mL) and it was stirred at RT overnight. The reaction mixture was evaporated, dissolved in water (3 mL), acidified with 1 M HCl solution to pH=5-6 and lyophilized to get the product (142 mg, crude). MS: M/e 274 (M+1)⁺

### Step 6: N-(3-((1s,3R)-3-methyl-1-(4-methyl-4H-1,2,4-triazol-3-yl)cyclobutyl) phenyl)-6-(((S)-3-methylpiperidin-1-yl)methyl)imidazo[1,2-a]pyridine-8-carboxamide

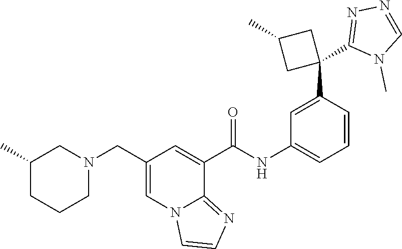 [SMILES: C[C@H]1CCCN(Cc2cc(C(=O)Nc3cccc([C@]4(c5nncn5C)C[C@@H](C)C4)c3)c3nccn3c2)C1]

A solution of (S)-6-((3-methylpiperidin-1-yl)methyl)imidazo[1,2-a]pyridine-8-carboxylic acid (140 mg, 0.51 mmol) and 3-((1s, 3s)-3-methyl-1-(4-methyl-4H-1,2,4-triazol-3-yl)cyclobutyl)aniline (123 mg, 0.51 mmol) in DMF (12 mL) were added with HATU (233 mg, 0.61 mmol) and DIEA (129 mg, 1.0 mmol). The mixture was stirred at RT for 2 hrs, then it was quenched with water (5 mL), extracted with DCM (15 mL), and washed with brine (15 mL). The organic layer was dried, concentrated and purified by CombiFlash (DCM:MeOH=7%) to get the product (98 mg, 39%). ¹H NMR (400 MHz, CD₃OD) δ 8.76 (s, 1H), 8.31 (s, 1H), 8.17 (s, 1H), 8.00 (s, 1H), 7.88 (s, 1H), 7.74 (s, 1H), 7.65 (d, J=8.0 Hz, 1H), 7.41 (t, J=8.0 Hz, 1H), 7.20 (d, J=8.0 Hz, 1H), 4.24 (s, 2H), 3.41-3.35 (m, 2H), 3.32 (s, 3H), 2.97-2.93 (m, 2H), 2.78-2.57 (m, 4H), 2.48 (t, J=12.0 Hz, 1H), 1.97-1.73 (m, 4H), 1.15 (d, J=4.0 Hz, 4H), 0.97 (d, J=8.0 Hz, 3H) ppm; MS: M/e 498 (M+1)+

### Compound A2: N-(3-((1r,3S)-3-methyl-1-(4-methyl-4H-1,2,4-triazol-3-yl)cyclobutyl)phenyl)-6-(((S)-3-methylpiperidin-1-yl)methyl)imidazo[1,2-a]pyridine-8-carboxamide

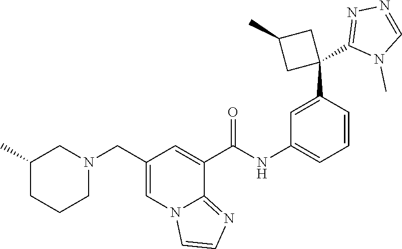 [SMILES: C[C@H]1CCCN(Cc2cc(C(=O)Nc3cccc([C@]4(c5nncn5C)C[C@H](C)C4)c3)c3nccn3c2)C1]

### Step A: 3-(3-methyl-1-(4-methyl-4H-1,2,4-triazol-3-yl)cyclobutyl)aniline

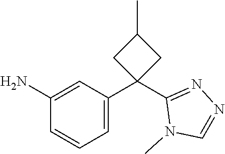 [SMILES: CC1CC(c2cccc(N)c2)(c2nncn2C)C1]

To a solution of 3-(1-(3-bromophenyl)-3-methylcyclobutyl)-4-methyl-4H-1,2,4-triazole (500 mg, 1.63 mmol) in ACN (10 mL)/NH₃H₂O (20 mL) was added Cu₂O (117 mg, 0.818 mmol) under N₂. The reaction mixture was stirred for 12 hrs at 100° C. After completed, the mixture was filtered and the filate was collected and concentrated under vacuum. The residue was purified by purified by column chromatography on silica gel eluting with methanol in dichloromethane (20%) to afford the title compound (400 mg, crude). MS: M/e 243 (M+H)⁺.

### Step B: (S)—N-(3-(3-methyl-1-(4-methyl-4H-1,2,4-triazol-3-yl)cyclobutyl)phenyl)-6-((3-methylpiperidin-1-yl)methyl)imidazo[1,2-a]pyridine-8-carboxamide

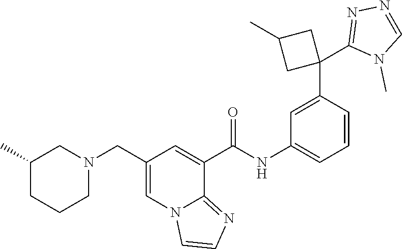 [SMILES: CC1CC(c2cccc(NC(=O)c3cc(CN4CCC[C@H](C)C4)cn4ccnc34)c2)(c2nncn2C)C1]

To a solution of (S)-6-((3-methylpiperidin-1-yl)methyl)imidazo[1,2-a]pyridine-8-carboxylic acid (30 mg, 0.109 mmol), 3-(3-methyl-1-(4-methyl-4H-1,2,4-triazol-3-yl)cyclobutyl)aniline (70 mg, crude), DIEA (28 mg, 0.217 mmol) in DMF (2 mL) was added HATU (50 mg, 0.132 mmol). The reaction mixture was stirred for 1 h at 25° C. After completed, the reaction mixture was quenched with water (10 mL) and extracted with DCM/MeOH (10/1, 3×30 mL). The combined organic layers were dried over Na₂SO₄ and concentrated under vacuum to get a residue. The residue was purified by Prep-TLC (DCM/MeOH=15/1 to 10/1) to afford the title compound (40 mg, impurity). MS: M/e 498 (M+H)⁺.

### Step C: N-(3-((1r,3S)-3-methyl-1-(4-methyl-4H-1,2,4-triazol-3-yl)cyclobutyl)phenyl)-6-(((S)-3-methylpiperidin-1-yl)methyl)imidazo[1,2-a]pyridine-8-carboxamide

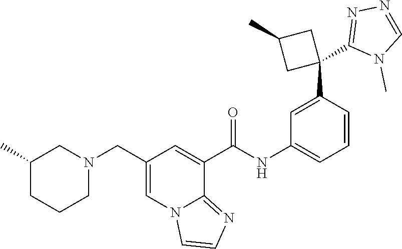 [SMILES: C[C@H]1CCCN(Cc2cc(C(=O)Nc3cccc([C@]4(c5nncn5C)C[C@H](C)C4)c3)c3nccn3c2)C1]

(S)—N-(3-(3-methyl-1-(4-methyl-4H-1,2,4-triazol-3-yl)cyclobutyl)phenyl)-6-((3-methylpiperidin-1-yl)methyl)imidazo[1,2-a]pyridine-8-carboxamide was re-purified by Prep-HPLC (NH₃H₂O condition) to afford N-(3-((1r,3S)-3-methyl-1-(4-methyl-4H-1,2,4-triazol-3-yl)cyclobutyl)phenyl)-6-(((S)-3-methylpiperidin-1-yl)methyl)imidazo[1,2-a]pyridine-8-carboxamide (3 mg). ¹H NMR (400 MHz, CD₃OD) δ 8.60 (s, 1H), 8.37 (s, 1H), 8.22 (s, 1H), 7.96 (s, 1H), 7.77 (s, 1H), 7.72 (s, 1H), 7.66 (d, J=9.0 Hz, 1H), 7.40 (t, J=8.0 Hz, 1H), 6.99 (d, J=7.8 Hz, 1H), 3.60 (s, 2H), 3.32 (s, 3H), 3.13 (t, J=9.4 Hz, 2H), 2.93-2.83 (m, 2H), 2.55-2.48 (m, 1H), 2.38 (t, J=10.2 Hz, 2H), 2.00 (t, J=10.0 Hz, 1H), 1.77-1.56 (m, 5H), 1.17 (d, J=6.4 Hz, 3H), 0.97-0.84 (m, 4H); MS: M/e 498 (M+H)⁺.

### Compound A3: N-(3-((1s,3R)-3-(cyanomethyl)-1-(4-methyl-4H-1,2,4-triazol-3-yl)cyclobutyl)phenyl)-6-(((S)-3-methylpiperidin-1-yl)methyl)imidazo[1,2-a]pyridine-8-carboxamide

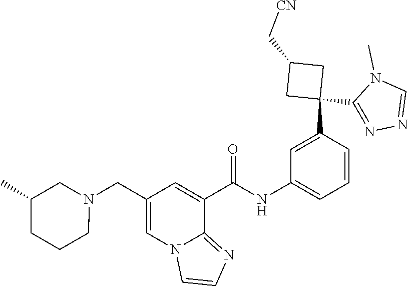 [SMILES: C[C@H]1CCCN(Cc2cc(C(=O)Nc3cccc([C@]4(c5nncn5C)C[C@@H](CC#N)C4)c3)c3nccn3c2)C1]

To a solution of 2-((1s, 3s)-3-(3-bromophenyl)-3-(4-methyl-4H-1,2,4-triazol-3-yl)cyclobutyl)acetonitrile (30 mg, 91 umol) in dioxane (3 mL) was added (S)-6-((3-methylpiperidin-1-yl)methyl)imidazo[1,2-a]pyridine-8-carboxamide (25 mg, 91 umol), tBuXPhospd-G3 (7.9 mg, 9.1 umol), tBuXPhos (3.8 mg, 9.1 umol) and t-BuONa (18 mg, 182 umol) at 15° C. under N₂. The mixture was stirred for 12 hrs at 80° C. under N₂. The mixture was concentrated to give the residue, which was purified by Prep-HPLC (water (NH₄HCO₃)-ACN, C18 100*25 mm*5 um) to give the title product (3.7 mg); ¹H NMR (400 MHz, CDCl₃-d) δ 12.5 (s, 1H), 8.12 (s, 1H), 7.99-8.02 (m, 2H) 7.94 (s, 1H) 7.76 (s, 1H) 7.67-7.69 (d, J=8 Hz, 1H) 7.37-7.41 (t, J=8 Hz, 1H) 7.04-7.06 (d, J=8 Hz, 1H) 4.27 (br s, 2H) 3.39 (s, 2H) 3.23-2.26 (m, 3H) 3.00-3.10 (m, 3H) 2.82-2.90 (m, 3H) 2.57-2.58 (m, 3H) 2.49 (m, 1H) 1.85-1.90 (m, 3H) 0.94-0.95 (m, 4H) ppm; MS: M/e 523 (M+H)⁺.

### Compound A4: N-(3-((1r,3S)-3-cyano-1-((4-methyl-4H-1,2,4-triazol-3-yl)methyl)cyclobutyl)phenyl)-6-(((S)-3-methylpiperidin-1-yl)methyl)imidazo[1,2-a]pyridine-8-carboxamide

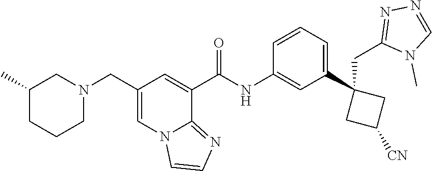 [SMILES: C[C@H]1CCCN(Cc2cc(C(=O)Nc3cccc([C@]4(Cc5nncn5C)C[C@@H](C#N)C4)c3)c3nccn3c2)C1]

A solution of (1r, 3r)-3-(3-aminophenyl)-3-((4-methyl-4H-1,2,4-triazol-3-yl) methyl)cyclobutane-1-carbonitrile (10 mg, 0.04 mmol) and (S)-6-((3-methylpiperidin-1-yl)methyl)imidazo[1,2-a]pyridine-8-carboxylic acid (12 mg, 0.04 mmol) in DMF (2 mL) were added with HATU (23 mg, 0.06 mmol) and DIEA (26 mg, 0.2 mmol). The mixture was stirred at RT for 1 hour, then it was quenched with water (3 mL), extracted with DCM (10 mL). The organic layer was dried, concentrated and purified by Prep-HPLC (Mobile Phase A: 0.1% FA-H₂O, Mobile Phase B: 0.1% FA-ACN) to get the product (5 mg, 24%). ¹H NMR (400 MHz, CD₃OD) δ 8.68 (s, 1H), 8.23 (s, 1H), 8.16 (s, 1H), 8.00 (s, 1H), 7.76 (s, 1H), 7.68 (d, J=8.0 Hz, 1H), 7.48 (s, 1H), 7.34 (t, J=8.0 Hz, 1H), 6.83 (d, J=8.0 Hz, 1H), 3.85 (s, 2H), 3.42 (s, 2H), 3.23-3.15 (m, 1H), 3.10-3.05 (m, 1H), 3.01 (s, 3H), 2.99 (s, 2H), 2.81 (s, 3H), 2.29 (t, J=12.0 Hz, 1H), 2.00 (t, J=12.0 Hz, 1H), 1.79-1.62 (m, 4H), 1.05-0.96 (m, 1H), 0.91 (d, J=8.0 Hz, 3H) ppm. MS: M/e 523 (M+1)⁺

### Compound A5: N-(3-((1s,3S)-3-(cyanomethyl)-1-(4-methyl-4H-1,2,4-triazol-3-yl)cyclobutyl)phenyl)-6-(((R)-2-methylmorpholino)methyl)imidazo[1,2-a]pyridine-8-carboxamide

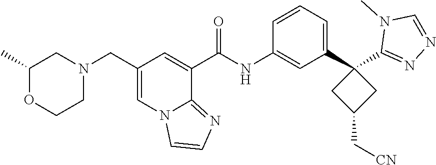 [SMILES: C[C@@H]1CN(Cc2cc(C(=O)Nc3cccc([C@]4(c5nncn5C)C[C@@H](CC#N)C4)c3)c3nccn3c2)CCO1]

Step A: methyl (R)-6-((2-methylmorpholino)methyl)imidazo[1,2-a]pyridine-8-carboxylate

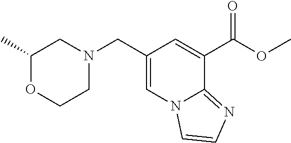

To a solution of methyl 6-formylimidazo[1,2-a]pyridine-8-carboxylate (100 mg, 0.49 mmol), (R)-2-methylmorpholine (98 mg, 0.97 mmol), AcOH (40 uL) in DCM (10 mL) was added NaBH(OAc)₃ (207 mg, 0.976 mmol). The reaction mixture was stirred for 12 hrs at 25° C. After completed, the reaction was quenched with aq NaHCO₃ (20 mL) and extracted with DCM/MeOH (10/1, 3×30 mL), dried over Na₂SO₄ and concentrated under vacuum. The residue was purified by Prep-TLC (DCM/MeOH=15/1) to afford the title compound (85 mg, 63%). MS: M/e 290 (M+H)⁺.

### Step B: (R)-6-((2-methylmorpholino)methyl)imidazo[1,2-a]pyridine-8-carboxylic acid

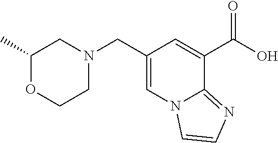 [SMILES: C[C@@H]1CN(Cc2cc(C(=O)O)c3nccn3c2)CCO1]

To a solution of methyl (R)-6-((2-methylmorpholino)methyl)imidazo[1,2-a]pyridine-8-carboxylate (20 mg, 0.069 mmol) in MeOH (5 mL)/H₂O (0.5 mL) was added LiOH H₂O (15 mg, 0.357 mmol). The reaction mixture was stirred for 12 hrs at 25° C. After completed, the solvent was concentrated under vacuum. The residue was diluted with water and neutralized with 2 N HCl and freed dried to afford the title compound (20 mg, crude). MS: M/e 276 (M+H)⁺.

### Step C: N-(3-((1s,3S)-3-(cyanomethyl)-1-(4-methyl-4H-1,2,4-triazol-3-yl)cyclobutyl)phenyl)-6-(((R)-2-methylmorpholino)methyl)imidazo[1,2-a]pyridine-8-carboxamide

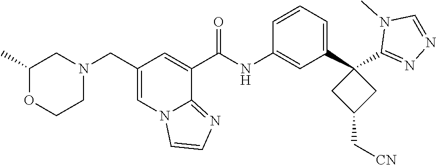 [SMILES: C[C@@H]1CN(Cc2cc(C(=O)Nc3cccc([C@]4(c5nncn5C)C[C@@H](CC#N)C4)c3)c3nccn3c2)CCO1]

To a solution of (R)-6-((2-methylmorpholino)methyl)imidazo[1,2-a]pyridine-8-carboxylic acid (20 mg, crude), 2-((1s, 3s)-3-(3-aminophenyl)-3-(4-methyl-4H-1,2,4-triazol-3-yl)cyclobutyl)acetonitrile (19 mg, 0.071 mmol), DIEA (18 mg, 0.139 mmol) in DMF (3 mL) was added HATU (40 mg, 0.105 mmol). The reaction mixture was stirred for 1 h at 25° C. After completed, the reaction was quenched with aq NH₄Cl (10 mL) and extracted with DCM/MeOH (10/1, 3×30 mL), dried over Na₂SO₄ and concentrated under vacuum. The residue was purified by Prep-HPLC to afford the title compound (15 mg, 36%). ¹H NMR (400 MHz, CD₃OD) δ 8.62 (s, 1H), 8.33 (s, 1H), 8.23 (s, 1H), 7.96 (s, 2H), 7.72 (s, 2H), 7.45 (t, J=8.0 Hz, 1H), 7.19 (d, J=8.0 Hz, 1H), 3.84 (d, J=11.6 Hz, 1H), 3.72-3.64 (m, 2H), 3.63 (s, 2H), 3.34 (s, 3H), 3.09-3.00 (m, 2H), 3.00-2.90 (m, 1H), 2.90-2.72 (m, 4H), 2.67 (d, J=5.8 Hz, 2H), 2.29-2.19 (m, 1H), 1.93 (t, J=10.8 Hz, 1H), 1.11 (d, J=6.4 Hz, 3H) ppm. MS: M/e 525 (M+H)⁺.

### Compound A6: 3-fluoro-6-{[(3S)-3-methylpiperidin-1-yl]methyl}-N-{3-[(1r, 3s)-3-methyl-1-(4-methyl-1,2,4-triazol-3-yl)cyclobutyl]phenyl}imidazo[1,2-a]pyridine-8-carboxamide

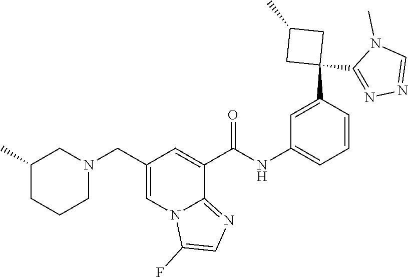 [SMILES: C[C@H]1CCCN(Cc2cc(C(=O)Nc3cccc([C@]4(c5nncn5C)C[C@@H](C)C4)c3)c3ncc(F)n3c2)C1]

### Step 1: 6-bromo-3-fluoroimidazo[1,2-a]pyridine-8-carboxylate

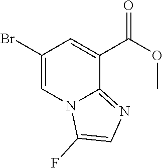

To a solution of methyl 6-bromoimidazo[1,2-a]pyridine-8-carboxylate (5 g, 18.622 mmol, 1 equiv) in THF was added sodium hydride (60% in oil, 1.2 g) at 0 degrees C. The mixture was stirred for 15 min. Selectfluor (20 g, 53.633 mmol, 2.88 equiv,) was added in portions at 0 degrees C. and the mixture was allowed to warm to RT and stirred for 10 min. The resulting mixture was stirred for 40 h at 60° C. under nitrogen atmosphere. The reaction mixture was quenched by water (40 mL) at 0 degrees C. and extracted with EA (3*130 mL). The organic layers were concentrated under vacuum. The residue was purified by silica gel column chromatography, eluted with PE/EA (100:0-0:100) to afford methyl 6-bromo-3-fluoroimidazo[1,2-a]pyridine-8-carboxylate (3.8 g). MS: M/e 273 (M+1)⁺.

### Step 2: 6-ethenyl-3-fluoroimidazo[1,2-a]pyridine-8-carboxylate

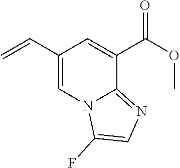 [SMILES: C=Cc1cc(C(=O)OC)c2ncc(F)n2c1]

To a solution of methyl 6-bromo-3-fluoroimidazo[1,2-a]pyridine-8-carboxylate (3.7 g, 12.710 mmol, 1 equiv) and 2-ethenyl-4,4,5,5-tetramethyl-1,3,2-dioxaborolane (4.2 g, 25.906 mmol, 2.04 equiv,) in dioxane (40 mL) and H2O (4 mL) were added K2CO3 (3.70 g, 25.420 mmol, 2.00 equiv) and Pd(dppf)Cl2CH2Cl2 (1 g, 1.166 mmol, 0.09 equiv,). The resulting mixture was stirred for 3 h at 100° C. under nitrogen atmosphere. The mixture was allowed to cool down to room temperature. The resulting mixture was concentrated under vacuum. The residue was dissolved in DCM (30 mL). The resulting mixture was filtered, the filter cake was washed with DCM (4×5 mL). The filtrate was concentrated under reduced pressure. The residue was purified by silica gel column chromatography, eluted with PE/EA (1:9) to afford methyl 6-ethenyl-3-fluoroimidazo[1,2-a]pyridine-8-carboxylate (1.9 g). MS: M/e 221 (M+1)⁺.

### Step 3: methyl 3-fluoro-6-formylimidazo[1,2-a]pyridine-8-carboxylate

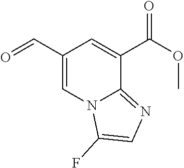 [SMILES: COC(=O)c1cc(C=O)cn2c(F)cnc12]

To a stirred mixture of methyl 6-ethenyl-3-fluoroimidazo[1,2-a]pyridine-8-carboxylate (1.8 g, 7.823 mmol, 1 equiv) in dioxane and H2O (12 mL,) was added K2OsO4·2H2O (303 mg, 0.781 mmol, 0.10 equiv,) and NaIO4 (3.6 g, 15.99 mmol, 2.04 equiv) in portions at 0° C. The resulting mixture was stirred for 3 h at room temperature. The reaction was quenched with Water (8 mL) at room temperature. The precipitated solids were collected by filtration and washed with water (3×1 mL). The water layers was extracted with EtOAc (3×80 mL). The combined organic layers were washed with brine (1×50 mL), dried over anhydrous Na2SO4. After filtration, the filtrate was concentrated under reduced pressure. This resulted in methyl 3-fluoro-6-formylimidazo[1,2-a]pyridine-8-carboxylate (1.2 g). MS: M/e 241 (M+1)⁺

### Step 4: methyl 3-fluoro-6-{[(3S)-3-methylpiperidin-1-yl]methyl}imidazo[1,2-a]pyridine-8-carboxylate

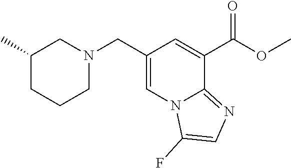

To a stirred mixture of methyl 3-fluoro-6-formylimidazo[1,2-a]pyridine-8-carboxylate (800 mg, 3.266 mmol, 1 equiv) and (3S)-3-methylpiperidine hydrochloride (932 mg, 6.528 mmol, 2.00 equiv, 95%) in DCE. The resulting mixture was stirred for 4 h at 70° C. The mixture was allowed to cool down to room temperature. To the above mixture was added STAB (1.1 g, 4.931 mmol, 1.51 equiv) in portions at room temperature. The resulting mixture was stirred for additional 3 h at 40° C. The resulting mixture was concentrated under vacuum. The residue was purified by reverse flash chromatography with the following conditions: column, C18 silica gel; mobile phase, MeCN in Water (0.1% NH3·H2O), 30% to 60% gradient in 30 min; detector, UV 220 nm. This resulted in methyl 3-fluoro-6-{[(3S)-3-methylpiperidin-1-yl]methyl}imidazo[1,2-a]pyridine-8-carboxylate and methyl (S)-2-fluoro-6-((3-methylpiperidin-1-yl)methyl)imidazo[1,2-a]pyridine-8-carboxylate (670 mg, 61.00%). MS: M/e 306 (M+1)⁺.

### Step 5: 3-fluoro-6-{[(3S)-3-methylpiperidin-1-yl]methyl}imidazo[1,2-a]pyridine-8-carboxylic acid

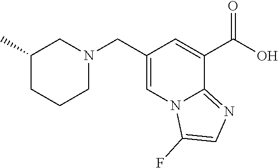 [SMILES: C[C@H]1CCCN(Cc2cc(C(=O)O)c3ncc(F)n3c2)C1]

To a stirred solution of methyl 3-fluoro-6-{[(3S)-3-methylpiperidin-1-yl]methyl}imidazo[1,2-a]pyridine-8-carboxylate and methyl (S)-2-fluoro-6-((3-methylpiperidin-1-yl)methyl)imidazo[1,2-a]pyridine-8-carboxylate (630 mg, 1.873 mmol, 1 equiv, 90.8%) in THF and H2O (1.5 mL, 99%) was added LiOH (142 mg, 5.633 mmol, 3.01 equiv, 95%). The resulting mixture was stirred for 15 h at room temperature. The resulting mixture was concentrated under vacuum. The residue was acidified to pH=3 with HCl (1 M). The residue was purified by reverse flash chromatography with the following conditions: column, C18 silica gel; mobile phase, MeCN in Water (0.1% FA), 0%; detector, UV 220 nm. This resulted in 3-fluoro-6-{[(3S)-3-methylpiperidin-1-yl]methyl}imidazo[1,2-a]pyridine-8-carboxylic acid (350 mg, 59.45%). MS: M/e 292 (M+1)⁺, and (S)-2-fluoro-6-((3-methylpiperidin-1-yl)methyl)imidazo[1,2-a]pyridine-8-carboxylic acid (170 mg, 26.94%). MS: M/e 292 (M+1)⁺.

### Step 6: 3-fluoro-6-{[(3S)-3-methylpiperidin-1-yl]methyl}-N-{3-[(1r, 3s)-3-methyl-1-(4-methyl-1,2,4-triazol-3-yl)cyclobutyl]phenyl}imidazo[1,2-a]pyridine-8-carboxamide

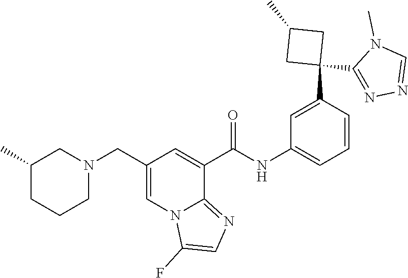 [SMILES: C[C@H]1CCCN(Cc2cc(C(=O)Nc3cccc([C@]4(c5nncn5C)C[C@@H](C)C4)c3)c3ncc(F)n3c2)C1]

To a stirred mixture of 3-fluoro-6-{[(3S)-3-methylpiperidin-1-yl]methyl}imidazo[1,2-a]pyridine-8-carboxylic acid (39 mg, 0.124 mmol, 1.23 equiv) and EDC·HCl (31 mg, 0.154 mmol, 1.52 equiv) and HOBT (22 mg, 0.155 mmol, 1.53 equiv) and Et3N (44 mg, 0.413 mmol, 4.08 equiv) in DMF was stirred for 5 min. To the above mixture was added 3-[(1r, 3s)-3-methyl-1-(4-methyl-1,2,4-triazol-3-yl)cyclobutyl]aniline (30 mg, 0.101 mmol, 1.00 equiv) in DMF dropwise at room temperature. The resulting mixture was stirred for additional 3 h at 40° C. The residue was purified by reverse flash chromatography with the following conditions: column, C18 silica gel; mobile phase, MeCN in Water (0.1% NH3·H2O), 0% to 80% gradient in 20 min; detector, UV 220 nm. The crude product (50 mg) was purified by Prep-HPLC with the following conditions (2 #SHIMADZU (HPLC-01)): Column, XBridge Prep OBD C18 Column, 30*150 mm, 5 μm; mobile phase, Water (10 mmol/L NH4HCO3+0.1% NH3·H2O) and ACN (49% ACN up to 79% in 8 min); This resulted in 3-fluoro-6-{[(3S)-3-methylpiperidin-1-yl]methyl}-N-{3-[(1r, 3s)-3-methyl-1-(4-methyl-1,2,4-triazol-3-yl)cyclobutyl]phenyl}imidazo[1,2-a]pyridine-8-carboxamide (22.1 mg, 41.56%). MS: M/e 516 (M+1)⁺; ¹H NMR (400 MHz, DMSO-d₆) δ 11.95 (s, 1H), 8.46 (s, 1H), 8.30 (s, 1H), 8.10 (d, J=1.6 Hz, 1H), 7.72 (d, J=1.9 Hz, 1H), 7.68-7.59 (m, 2H), 7.46-7.38 (m, 1H), 7.17 (d, J=7.8 Hz, 1H), 3.55 (s, 2H), 3.20 (s, 3H), 2.87-2.73 (m, 4H), 2.55 (d, J=6.8 Hz, 3H), 1.98-1.88 (m, 1H), 1.68-1.56 (m, 4H), 1.47 (d, J=12.3 Hz, 1H), 1.09 (d, J=5.0 Hz, 3H), 0.82 (d, J=5.9 Hz, 4H) ppm.

### Compound A7: (S)-2-fluoro-N-(3-(3-((4-methyl-4H-1,2,4-triazol-3-yl)methyl)oxetan-3-yl)phenyl)-6-((3-methylpiperidin-1-yl)methyl)imidazo[1,2-a]pyridine-8-carboxamide

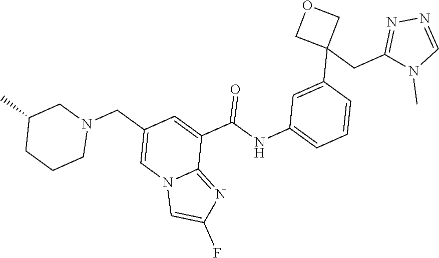 [SMILES: C[C@H]1CCCN(Cc2cc(C(=O)Nc3cccc(C4(Cc5nncn5C)COC4)c3)c3nc(F)cn3c2)C1]

To a stirred mixture of (S)-2-fluoro-6-((3-methylpiperidin-1-yl)methyl)imidazo[1,2-a]pyridine-8-carboxylic acid (42 mg, 0.125 mmol, 1.22 equiv,) and HOBT (22 mg, 0.155 mmol, 1.51 equiv) and EDC·HCl (31 mg, 0.154 mmol, 1.50 equiv) in DMF was added Et3N (44 mg, 0.413 mmol, 4.04 equiv) dropwise at room temperature. After 5 mins, To the above mixture was added 3-{3-[(4-methyl-1,2,4-triazol-3-yl)methyl]oxetan-3-yl}aniline (25 mg, 0.102 mmol, 1.00 equiv) dropwise at room temperature. The resulting mixture was stirred for additional 3 h at 40° C. The residue was purified by reverse flash chromatography with the following conditions: column, C18 silica gel; mobile phase, MeCN in Water (0.1% NH3·H2O), 0% to 95% gradient in 25 min; detector, UV 220 nm. The crude product (50 mg) was purified by Prep-HPLC with the following conditions (2 #SHIMADZU (HPLC-01)): Column, XBridge Shield RP18 OBD Column, 30*150 mm, 5 μm; mobile phase, Water (10 mmol/L NH4HCO3+0.1% NH3·H2O) and ACN (31% ACN up to 61% in 8 min); This resulted in (S)-2-fluoro-N-(3-(3-((4-methyl-4H-1,2,4-triazol-3-yl)methyl)oxetan-3-yl)phenyl)-6-((3-methylpiperidin-1-yl)methyl)imidazo[1,2-a]pyridine-8-carboxamide (24 mg, 44.95%). MS: M/e 518 (M+H)⁺; ¹H NMR (400 MHz, DMSO-d₆) δ 11.27 (s, 1H), 8.69 (d, J=1.6 Hz, 1H), 8.20 (s, 1H), 8.11 (d, J=1.6 Hz, 1H), 7.94 (d, J=7.6 Hz, 1H), 7.67-7.60 (m, 1H), 7.40-7.35 (m, 1H), 7.35-7.28 (m, 1H), 6.74-6.67 (m, 1H), 4.96 (d, J=6.0 Hz, 2H), 4.87 (d, J=6.0 Hz, 2H), 3.59-3.47 (m, 4H), 2.94 (s, 3H), 2.76 (t, J=10.1 Hz, 2H), 1.97-1.87 (m, 1H), 1.70-1.55 (m, 4H), 1.54-1.40 (m, 1H), 0.92-0.78 (m, 4H) ppm.

### Compound A8: 2-fluoro-N-(3-((1s,3R)-3-methyl-1-(4-methyl-4H-1,2,4-triazol-3-yl)cyclobutyl)phenyl)-6-(((S)-3-methylpiperidin-1-yl)methyl)imidazo[1,2-a]pyridine-8-carboxamide

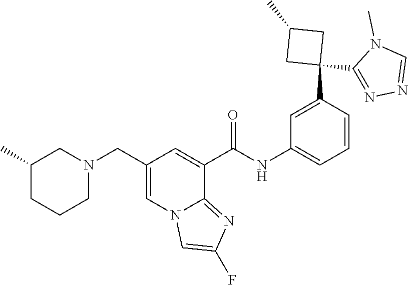 [SMILES: C[C@H]1CCCN(Cc2cc(C(=O)Nc3cccc([C@]4(c5nncn5C)C[C@@H](C)C4)c3)c3nc(F)cn3c2)C1]

### Step 1: (S)-2-fluoro-6-((3-methylpiperidin-1-yl)methyl)imidazo[1,2-a]pyridine-8-carboxylic acid

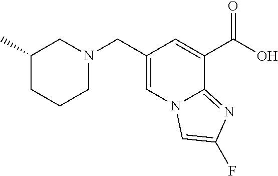 [SMILES: C[C@H]1CCCN(Cc2cc(C(=O)O)c3nc(F)cn3c2)C1]

To a stirred solution of methyl 3-fluoro-6-{[(3S)-3-methylpiperidin-1-yl]methyl}imidazo[1,2-a]pyridine-8-carboxylate and methyl (S)-2-fluoro-6-((3-methylpiperidin-1-yl)methyl)imidazo[1,2-a]pyridine-8-carboxylate (630 mg, 1.873 mmol, 1 equiv, 90.8%) in THF and H2O (1.5 mL, 99%) was added LiOH (142 mg, 5.633 mmol, 3.01 equiv, 95%). The resulting mixture was stirred for 15 h at room temperature. The resulting mixture was concentrated under vacuum. The residue was acidified to pH 3 with HCl (1 M). The residue was purified by reverse flash chromatography with the following conditions: column, C18 silica gel; mobile phase, MeCN in Water (0.1% FA), 0%; detector, UV 220 nm. This resulted in 3-fluoro-6-{[(3S)-3-methylpiperidin-1-yl]methyl}imidazo[1,2-a]pyridine-8-carboxylic acid (350 mg, 59.45%). MS: M/e 292 (M+H)⁺, and (S)-2-fluoro-6-((3-methylpiperidin-1-yl)methyl)imidazo[1,2-a]pyridine-8-carboxylic acid (170 mg, 26.94%). MS: M/e 292 (M+H)⁺.

### Step 2: 2-fluoro-N-(3-((1s,3R)-3-methyl-1-(4-methyl-4H-1,2,4-triazol-3-yl)cyclobutyl)phenyl)-6-(((S)-3-methylpiperidin-1-yl)methyl)imidazo[1,2-a]pyridine-8-carboxamide

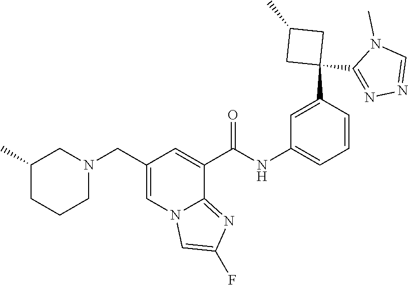 [SMILES: C[C@H]1CCCN(Cc2cc(C(=O)Nc3cccc([C@]4(c5nncn5C)C[C@@H](C)C4)c3)c3nc(F)cn3c2)C1]

To a stirred mixture of (S)-2-fluoro-6-((3-methylpiperidin-1-yl)methyl)imidazo[1,2-a]pyridine-8-carboxylic acid (38 mg, 0.113 mmol, 1.19 equiv) and HOBT (21 mg, 0.148 mmol, 1.56 equiv) and EDC·HCl (29 mg, 0.144 mmol, 1.52 equiv) in DMF was added Et3N (41 mg, 0.385 mmol, 4.07 equiv) dropwise at room temperature. After 5 mins, To the above mixture was added 3-[(1r, 3s)-3-methyl-1-(4-methyl-1,2,4-triazol-3-yl)cyclobutyl]aniline (28 mg, 0.095 mmol, 1.00 equiv) dropwise at room temperature. The resulting mixture was stirred for additional 3 h at 40° C. The residue was purified by reverse flash chromatography with the following conditions: column, C18 silica gel; mobile phase, MeCN in Water (0.1% NH3·H2O), 0% to 95% gradient in 25 min; detector, UV 220 nm. The crude product (45 mg) was purified by Prep-HPLC with the following conditions (2 #SHIMADZU (HPLC-01)): Column, XBridge Prep OBD C18 Column, 30*150 mm, 5 μm; mobile phase, Water (10 mmol/L NH4HCO3+0.1% NH3·H2O) and ACN (49% ACN up to 79% in 8 min); This resulted in 2-fluoro-N-(3-((1s,3R)-3-methyl-1-(4-methyl-4H-1,2,4-triazol-3-yl)cyclobutyl)phenyl)-6-(((S)-3-methylpiperidin-1-yl)methyl)imidazo[1,2-a]pyridine-8-carboxamide (18.9 mg). MS: M/e 516 (M+H)⁺. ¹H NMR (400 MHz, DMSO-d₆) δ 11.28 (s, 1H), 8.68 (d, J=1.6 Hz, 1H), 8.31 (s, 1H), 8.09 (d, J=1.7 Hz, 1H), 7.93 (d, J=7.6 Hz, 1H), 7.76 (t, J=2.0 Hz, 1H), 7.64-7.57 (m, 1H), 7.42 (t, J=7.9 Hz, 1H), 7.19-7.12 (m, 1H), 3.52 (d, J=3.1 Hz, 2H), 3.20 (s, 3H), 2.89-2.71 (m, 4H), 2.55 (d, J=6.7 Hz, 3H), 1.92 (t, J=10.9 Hz, 1H), 1.63-1.58 (m, 4H), 1.48 (d, J=12.4 Hz, 1H), 1.09 (d, J=4.7 Hz, 3H), 0.82 (d, J=5.7 Hz, 4H) ppm.

### Compound A9: 6-((cyclohexylamino)methyl)-N-(3-(3-((4-methyl-4H-1,2,4-triazol-3-yl)methyl)oxetan-3-yl)phenyl)imidazo[1,2-a]pyridine-8-carboxamide

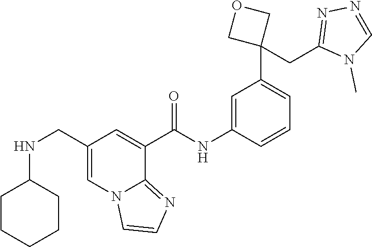 [SMILES: Cn1cnnc1CC1(c2cccc(NC(=O)c3cc(CNC4CCCCC4)cn4ccnc34)c2)COC1]

### Step 1: N-(3-(3-((4-methyl-4H-1,2,4-triazol-3-yl)methyl)oxetan-3-yl)phenyl)-6-vinylimidazo[1,2-a]pyridine-8-carboxamide

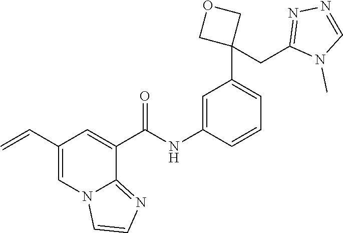 [SMILES: C=Cc1cc(C(=O)Nc2cccc(C3(Cc4nncn4C)COC3)c2)c2nccn2c1]

To a solution of lithium 6-vinylimidazo[1,2-a]pyridine-8-carboxylate (2.12 g, 8.19 mmol) in DMF (20 mL) was added DIEA (2.12 g, 16.37 mmol) and HATU (3.74 g, 9.82 mmol). The mixture was stirred for 5 mins at rt, then 3-(3-((4-methyl-4H-1,2,4-triazol-3-yl)methyl)oxetan-3-yl)aniline (2 g, 8.19 mmol) was added. The mixture was stirred for 12 hrs at rt. The mixture was poured into water (10 mL) and extracted with DCM (30 mL, 10 mL). The combined organic phase was filtered and concentrated to give the residue, which was purified by column chromatography (SiO₂, EA/MeOH=100/1 to 0/1) to give the product (2.1 mg, 61.89% yield). MS: M/e 415 (M+1)⁺; ¹H NMR (400 MHz, DMSO-d₆) δ=12.31 (s, 1H), 8.91 (d, J=1.6 Hz, 1H), 8.33 (d, J=4.0 Hz, 1H), 8.21 (s, 1H), 8.14 (d, J=4.0 Hz, 1H), 7.81 (d, J=4.0 Hz, 1H), 7.69 (dd, J=4.0, 8.0 Hz, 1H), 7.38 (t, J=4.0 Hz, 1H), 7.32 (t, J=8.0 Hz, 1H), 6.84 (dd, J=8.0, 16.0 Hz, 1H), 6.70 (d, J=8.0 Hz, 1H), 5.96 (d, J=20.0 Hz, 1H), 5.43 (d, J=8.0 Hz, 1H), 4.96 (d, J=4.0 Hz, 2H), 4.88 (d, J=4.0 Hz, 2H), 3.52 (s, 2H), 2.94 (s, 3H) ppm.

### Step 2: 6-formyl-N-(3-(3-((4-methyl-4H-1,2,4-triazol-3-yl)methyl)oxetan-3-yl)phenyl)imidazo[1,2-a]pyridine-8-carboxamide

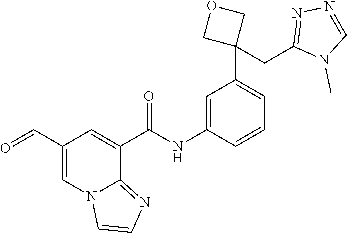 [SMILES: Cn1cnnc1CC1(c2cccc(NC(=O)c3cc(C=O)cn4ccnc34)c2)COC1]

To a mixture of N-(3-(3-((4-methyl-4H-1,2,4-triazol-3-yl)methyl)oxetan-3-yl)phenyl)-6-vinylimidazo[1,2-a]pyridine-8-carboxamide (2.10 g, 5.07 mmol) and K₂OsO₄H₂O (186.68 mg, 506.67 mol) in Dioxane (40 mL) and H₂O (8 mL) was added NaIO₄ (4.33 g, 20.27 mmol) in one portion at 20° C. under N₂. The mixture was stirred at 20° C. and stirred for 16 hours. The reaction mixture was quenched by addition H₂O 30 mL at 20° C., and then extracted with DCM (60 mL, 20 mL). The combined organic layers were washed with brine (60 mL, 20 mL), dried over Na₂SO₄, filtered and concentrated under reduced pressure to give the crude product (1.7 g, crude), which was used to the next step without purification. MS: M/e 417 (M+1); ¹H NMR (400 MHz, DMSO-d₆) δ=12.15 (s, 1H), 10.06 (s, 1H), 9.58 (d, J=1.6 Hz, 1H), 8.42 (d, J=1.6 Hz, 1H), 8.38 (d, J=1.6 Hz, 1H), 8.20 (s, 1H), 7.96 (d, J=1.6 Hz, 1H), 7.78-7.67 (m, 1H), 7.41-7.24 (m, 2H), 6.72 (d, J=8.0 Hz, 1H), 4.96 (d, J=8.0 Hz, 2H), 4.88 (d, J=4.0 Hz, 2H), 3.57 (s, 2H), 2.95 (s, 3H) ppm.

### Step 3: 6-((cyclohexylamino)methyl)-N-(3-(3-((4-methyl-4H-1,2,4-triazol-3-yl)methyl)oxetan-3-yl)phenyl)imidazo[1,2-a]pyridine-8-carboxamide

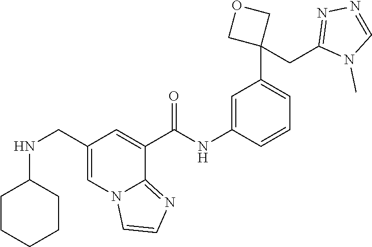 [SMILES: Cn1cnnc1CC1(c2cccc(NC(=O)c3cc(CNC4CCCCC4)cn4ccnc34)c2)COC1]

To a solution of 6-formyl-N-(3-(3-((4-methyl-4H-1,2,4-triazol-3-yl)methyl)oxetan-3-yl)phenyl)imidazo[1,2-a]pyridine-8-carboxamide (20 mg, 48.03 mol) and cyclohexanamine (4.76 mg, 48.03 mol) in DCM (1 mL). NaBH(OAc)₃ (20.36 mg, 96.05 mol) was added. The mixture was stirred for 16 hrs at rt under N₂. The mixture was poured into water (1 mL) and extracted with DCM (2 mL, 1 mL). The combined organic phase was washed with brine (1 mL), dried over Na₂SO₄, filtered and concentrated to give the residue, which was purified by prep-HPLC (column=Waters Xbridge BEH C18 (100*30 mm*10 um); mobile phase=water (NH₄HCO₃)-ACN, B %=20%-50%; 10 min) to give the product (4.42 mg, 34.6% yield). MS: M/e 500 (M+1); ¹H NMR (400 MHz, DMSO-d₆) δ=12.40 (s, 1H), 8.74 (s, 1H), 8.24-8.10 (m, 3H), 7.78 (s, 1H), 7.75-7.67 (m, 1H), 7.40-7.27 (m, 2H), 6.69 (br d, J=7.6 Hz, 1H), 4.96 (br d, J=6.0 Hz, 2H), 4.87 (br d, J=6.0 Hz, 2H), 3.82 (s, 2H), 3.52 (s, 3H), 2.94 (s, 3H), 2.40 (br s, 1H), 1.87 (br d, J=11.6 Hz, 2H), 1.66 (br s, 2H), 1.61-1.39 (m, 1H), 1.28-0.98 (m, 5H) ppm.

### Compound A10: 3-methyl-6-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}imidazo[1,2-a]pyridine-8-carboxamide

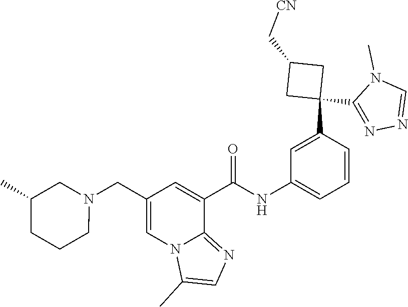 [SMILES: Cc1cnc2c(C(=O)Nc3cccc([C@]4(c5nncn5C)C[C@@H](CC#N)C4)c3)cc(CN3CCC[C@H](C)C3)cn12]

### Step 1: 2-bromopropanal

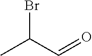

To a stirred solution of propionaldehyde (18 g, 294.421 mmol, 1 equiv) in 1,4-dioxane and DCM (3 mL) was added Br2 (15.88 mL, 294.421 mmol, 1 equiv) dropwise at 0° C. under nitrogen atmosphere. The resulting mixture was stirred for 1 h at 0° C. under nitrogen atmosphere. The reaction was quenched by the addition of sat. NaHCO₃(aq.) (50 mL) at 0° C. The aqueous layer was extracted with CH2Cl2 (2×50 mL). The resulting mixture was concentrated under reduced pressure. This resulted in 2-bromopropanal (30 g, 44.63%) as a yellow oil. The crude product was used in the next step directly without further purification.

### Step 2: methyl 6-bromo-3-methylimidazo[1,2-a]pyridine-8-carboxylate

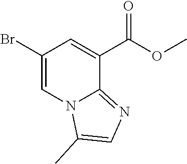

To a stirred solution of methyl 2-amino-5-bromopyridine-3-carboxylate (2 g, 8.223 mmol, 1 equiv) in EtOH was added 2-bromopropanal (7.51 g, 32.892 mmol, 4 equiv) at room temperature. The resulting mixture was stirred for overnight at 85° C. under nitrogen atmosphere. The mixture was allowed to cool down to room temperature. The resulting mixture was concentrated under reduced pressure. The residue was purified by silica gel column chromatography, eluted with CH2Cl2/MeOH (10:1) to afford methyl 6-bromo-3-methylimidazo[1,2-a]pyridine-8-carboxylate (2 g). MS: M/e 269 (M+H)⁺.

### Step 3: methyl 6-ethenyl-3-methylimidazo[1,2-a]pyridine-8-carboxylate

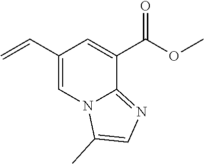 [SMILES: C=Cc1cc(C(=O)OC)c2ncc(C)n2c1]

To a stirred mixture of methyl 6-bromo-3-methylimidazo[1,2-a]pyridine-8-carboxylate (2 g, 6.927 mmol, 1 equiv) and 2-ethenyl-4,4,5,5-tetramethyl-1,3,2-dioxaborolane (2.25 g, 13.854 mmol, 2 equiv) in 1,4-dioxane and H2O (1 mL, 55.509 mmol, 8.01 equiv, 100%) were added K2CO3 (2.02 g, 13.854 mmol, 2 equiv) and Pd(dppf)Cl2CH2Cl2 (0.59 g, 0.693 mmol, 0.1 equiv) at room temperature. The resulting mixture was stirred for 2 h at 100° C. under nitrogen atmosphere. The mixture was allowed to cool down to room temperature. The resulting mixture was concentrated under reduced pressure. The residue was purified by silica gel column chromatography, eluted with CH2Cl2/MeOH (10:1) to afford methyl 6-ethenyl-3-methylimidazo[1,2-a]pyridine-8-carboxylate (1.3 g). MS: M/e 217 (M+H)⁺.

### Step 4: methyl 6-formyl-3-methylimidazo[1,2-a]pyridine-8-carboxylate

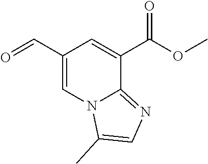 [SMILES: COC(=O)c1cc(C=O)cn2c(C)cnc12]

To a stirred solution of methyl 6-ethenyl-3-methylimidazo[1,2-a]pyridine-8-carboxylate (1.3 g, 5.609 mmol, 1 equiv) in dioxane and H2O (5 mL, 277.546 mmol, 49.48 equiv) was added K2OsO4·2H2O (217.54 mg, 0.561 mmol, 0.1 equiv) and NaIO4 (2525.72 g, 11218.075 mmol, 2000.00 equiv) at room temperature. The resulting mixture was stirred for 3 h at room temperature. The resulting mixture was diluted with MeOH (10 mL). The resulting mixture was filtered, the filter cake was washed with MeOH (2×5 mL). The filtrate was concentrated under reduced pressure. The residue was purified by silica gel column chromatography, eluted with CH2Cl2/MeOH (9:1) to afford methyl 6-formyl-3-methylimidazo[1,2-a]pyridine-8-carboxylate (400 mg, 31.24%). MS: M/e 219 (M+H)⁺.

### Step 5: methyl 3-methyl-6-{[(3S)-3-methylpiperidin-1-yl]methyl}imidazo[1,2-a]pyridine-8-carboxylate

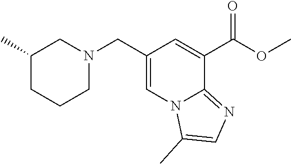

To a stirred mixture of methyl 6-formyl-3-methylimidazo[1,2-a]pyridine-8-carboxylate (400 mg, 1.752 mmol, 1 equiv) and (3S)-3-methylpiperidine hydrochloride (500.42 mg, 3.505 mmol, 2.00 equiv) in DCE. The resulting mixture was stirred for 3 h at 70° C. The mixture was allowed to cool down to room temperature. To the above mixture was added STAB (586.43 mg, 2.628 mmol, 1.5 equiv) in portions at room temperature. The resulting mixture was stirred for additional 3 h at 40° C. The resulting mixture was concentrated under vacuum. The residue was purified by silica gel column chromatography, eluted with CH2Cl2/MeOH (9:1) to afford methyl 3-methyl-6-{[(3S)-3-methylpiperidin-1-yl]methyl}imidazo[1,2-a]pyridine-8-carboxylate (200 mg). MS: M/e 302 (M+H)⁺.

### Step 6: 3-methyl-6-{[(3S)-3-methylpiperidin-1-yl]methyl}imidazo[1,2-a]pyridine-8-carboxylic acid

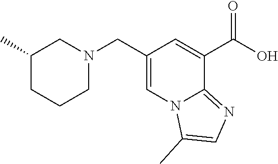 [SMILES: Cc1cnc2c(C(=O)O)cc(CN3CCC[C@H](C)C3)cn12]

To a stirred solution of methyl 3-methyl-6-{[(3S)-3-methylpiperidin-1-yl]methyl}imidazo[1,2-a]pyridine-8-carboxylate (200 mg, 0.516 mmol, 1 equiv, 77.7%) in THF and H2O (2 mL, 111.019 mmol, 215.31 equiv, 100%) was added LiOH (52.00 mg, 2.064 mmol, 4 equiv, 95%) at room temperature. The resulting mixture was stirred for 1 h at room temperature. The mixture was acidified to pH 6 with HCl (aq.). The resulting mixture was concentrated under reduced pressure. The residue was purified by reverse flash chromatography with the following conditions: column, C18 silica gel; mobile phase, MeOH in Water (10 mmol/L NH4HCO3), 30% to 40% gradient in 10 min; detector, UV 254 nm. This resulted in 3-methyl-6-{[(3S)-3-methylpiperidin-1-yl]methyl}imidazo[1,2-a]pyridine-8-carboxylic acid (90 mg, 60.68%). MS: M/e 288 (M+H)⁺.

### Step 7:3-methyl-6-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}imidazo[1,2-a]pyridine-8-carboxamide

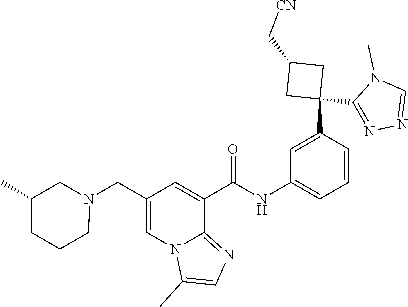 [SMILES: Cc1cnc2c(C(=O)Nc3cccc([C@]4(c5nncn5C)C[C@@H](CC#N)C4)c3)cc(CN3CCC[C@H](C)C3)cn12]

To a stirred mixture of 3-methyl-6-{[(3S)-3-methylpiperidin-1-yl]methyl}imidazo[1,2-a]pyridine-8-carboxylic acid (20.66 mg, 0.072 mmol, 1.2 equiv, 99.9%) and EDCI (20.53 mg, 0.102 mmol, 1.7 equiv, 95%) and HOBT (13.62 mg, 0.096 mmol, 1.6 equiv, 95%) and TEA (27.41 mg, 0.258 mmol, 4.3 equiv, 95%) in DMF and stirred for 5 min. To the above mixture was added 2-[(1s, 3s)-3-(3-aminophenyl)-3-(4-methyl-1,2,4-triazol-3-yl)cyclobutyl]acetonitrile (20 mg, 0.060 mmol, 1.00 equiv, 80%) in DMF at room temperature. The resulting mixture was stirred for additional 3 h at 40° C. The mixture was allowed to cool down to room temperature. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH4HCO3) and ACN (35% ACN up to 65% in 8 min); This resulted in 3-methyl-6-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}imidazo[1,2-a]pyridine-8-carboxamide (13.7 mg, 42.27%). MS: M/e 537 (M+H)⁺; ¹H NMR (400 MHz, DMSO-d₆) δ 12.45 (s, 1H), 8.44 (d, J=1.6 Hz, 1H), 8.32 (s, 1H), 8.11 (d, J=1.6 Hz, 1H), 7.80-7.75 (m, 1H), 7.66 (d, J=8.0, 2.0 Hz, 1H), 7.59 (s, 1H), 7.48-7.40 (m, 1H), 7.20 (d, J=7.6, 1.9 Hz, 1H), 3.56 (s, 2H), 3.22 (s, 3H), 2.94-2.66 (m, 9H), 2.53 (s, 3H), 1.98-1.87 (m, 1H), 1.70-1.54 (m, 4H), 1.54-1.41 (m, 1H), 0.93-0.79 (m, 4H) ppm.

### Compound A11: 5-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrazolo[1,5-a]pyridine-7-carboxamide

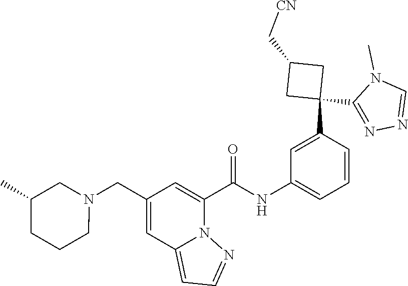 [SMILES: C[C@H]1CCCN(Cc2cc(C(=O)Nc3cccc([C@]4(c5nncn5C)C[C@@H](CC#N)C4)c3)n3nccc3c2)C1]

### Step 1: 5-ethenylpyrazolo[1,5-a]pyridine

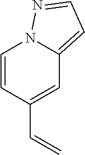

To a solution of 5-bromopyrazolo[1,5-a]pyridine (4 g, 19.286 mmol, 1 equiv) and 2-ethenyl-4,4,5,5-tetramethyl-1,3,2-dioxaborolane (3.64 g, 23.143 mmol, 1.20 equiv) in dioxane (40 mL) and H2O (4 mL) were added K2CO3 (5.61 g, 38.572 mmol, 2.00 equiv) and Pd(dppf)Cl2·CH2Cl2 (1.98 g, 2.314 mmol, 0.12 equiv). After stirring for 5 h at 100° C. under a nitrogen atmosphere. The resulting mixture was concentrated under reduced pressure. The residue was purified by silica gel column chromatography, eluted with CH2Cl2/PE (1:3) to afford 5-ethenylpyrazolo[1,5-a]pyridine (1.6 g, 35.45%). MS: M/e 145 (M+H)⁺.

### Step 2: 7-bromo-5-ethenylpyrazolo[1,5-a]pyridine

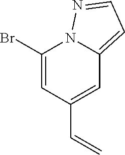

To a stirred solution of 5-ethenylpyrazolo[1,5-a]pyridine (150 mg, 0.678 mmol, 1 equiv) in THF was added 2,2,6,6-Tetramethylpiperidinylmagnesium chloride lithium chloride complex solution in (1M) THF (0.81 mL, 0.814 mmol, 1.2 equiv) dropwise at −78° C. under nitrogen atmosphere. The mixture was stirred for 30 min at −78° C. under nitrogen atmosphere. To the mixture was added 1,2-dibromo-1,1,2,2-tetrachloroethane (349 mg, 1.018 mmol, 1.50 equiv) in 20 mL THF dropwise at −78° C. The resulting mixture was stirred for additional 4 h at room temperature. The reaction was quenched by the addition of sat. NH4Cl (aq.) (20 mL) at 0° C. The resulting mixture was added water (100 mL). The resulting mixture was extracted with EtOAc (3×150 mL). The combined organic layers were dried over anhydrous Na2SO4. After filtration, the filtrate was concentrated under reduced pressure. The residue was purified by silica gel column chromatography, eluted with PE/EA (4:1) to afford 7-bromo-5-ethenylpyrazolo[1,5-a]pyridine (1.5 g, 89.71%). MS: M/e 223 (M+H)⁺.

### Step 3: 5-ethenylpyrazolo[1,5-a]pyridine-7-carbonitrile

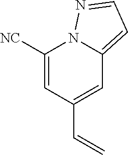

A solution of 7-bromo-5-ethenylpyrazolo[1,5-a]pyridine (1.5 g, 6.133 mmol, 1 equiv, 91.2%), Zn (42 mg, 0.610 mmol, 0.10 equiv, 95%), Zn(CN)₂ (1.21 g, 9.813 mmol, 1.6 equiv, 95%), XantPhos (1.49 g, 2.453 mmol, 0.40 equiv, 95%) and Pd(OAc)₂ (290 mg, 1.227 mmol, 0.20 equiv, 95%) in DMA was stirred for 1 h at 110° C. under nitrogen atmosphere. The resulting mixture was concentrated under reduced pressure. The residue was purified by silica gel column chromatography, eluted with PE/EA (5:1) to afford 5-ethenylpyrazolo[1,5-a]pyridine-7-carbonitrile (650 mg, 61.90%). MS: M/e 170 (M+H)⁺.

### Step 4: 5-formylpyrazolo[1,5-a]pyridine-7-carbonitrile

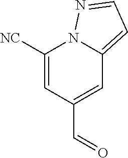 [SMILES: N#Cc1cc(C=O)cc2ccnn12]

To a stirred solution of 5-ethenylpyrazolo[1,5-a]pyridine-7-carbonitrile (650 mg, 3.796 mmol, 1 equiv) in dioxane (6 mL, 69.407 mmol, 18.29 equiv) and H2O (6 mL, 326.395 mmol, 85.99 equiv) were added K2OsO4·2H2O (147 mg, 0.379 mmol, 0.10 equiv) and NaIO4 (1.70 g, 7.551 mmol, 1.99 equiv) in portions at 0° C. The resulting mixture was stirred for 3 h at room temperature. The mixture was added H2O (20 mL). The resulting mixture was extracted with EtOAc (3×50 mL). The combined organic layers were dried over anhydrous Na2SO4. After filtration, the filtrate was concentrated under reduced pressure. The crude product was used in the next step directly without further purification. This resulted in 5-formylpyrazolo[1,5-a]pyridine-7-carbonitrile (600 mg, 85.70%). MS: M/e 172 (M+H)⁺.

### Step 5: 5-{[(3S)-3-methylpiperidin-1-yl]methyl}pyrazolo[1,5-a]pyridine-7-carbonitrile

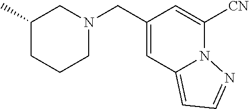 [SMILES: C[C@H]1CCCN(Cc2cc(C#N)n3nccc3c2)C1]

A solution of 5-formylpyrazolo[1,5-a]pyridine-7-carbonitrile (600 mg, 3.253 mmol, 1 equiv, 92.8%) and (3S)-3-methylpiperidine hydrochloride (687 mg, 4.812 mmol, 1.48 equiv) in DCE was stirred for 2 h at 70° C. under nitrogen atmosphere. The mixture was allowed to cool down to room temperature. To the reaction mixture was added STAB (1.08 g, 4.847 mmol, 1.49 equiv, 95%) in portions at room temperature. The resulting mixture was stirred for additional 2 h at 40° C. The mixture was allowed to cool down to room temperature. The resulting mixture was concentrated under reduced pressure. The residue was purified by reverse flash chromatography with the following conditions: column, C18 silica gel; mobile phase, MeCN in Water (0.1% TFA), 40% to 50% gradient in 5 min; detector, UV 220 nm. This resulted in 5-{[(3S)-3-methylpiperidin-1-yl]methyl}pyrazolo[1,5-a]pyridine-7-carbonitrile (600 mg, 64.98%). MS: M/e 255 (M+H)⁺.

### Step 6: 5-{[(3S)-3-methylpiperidin-1-yl]methyl}pyrazolo[1,5-a]pyridine-7-carboxylic acid

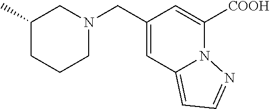 [SMILES: C[C@H]1CCCN(Cc2cc(C(=O)O)n3nccc3c2)C1]

To a stirred solution of 5-{[(3S)-3-methylpiperidin-1-yl]methyl}pyrazolo[1,5-a]pyridine-7-carbonitrile (600 mg, 2.114 mmol, 1 equiv, 89.6%) in EtOH (5 mL, 84.345 mmol, 39.90 equiv, 98%) and H2O (5 mL, 271.996 mmol, 128.68 equiv, 98%) was added NaOH (359 mg, 8.527 mmol, 4.03 equiv, 95%) in portions at 0° C. The resulting mixture was stirred for overnight at 85° C. under nitrogen atmosphere. The mixture was allowed to cool down to room temperature. The resulting mixture was concentrated under reduced pressure. The residue was purified by reverse flash chromatography with the following conditions: column, C18 silica gel; mobile phase, MeCN in Water (0.1% HCl), 15% to 30% gradient in 5 min; detector, UV 220 nm. This resulted in 5-{[(3S)-3-methylpiperidin-1-yl]methyl}pyrazolo[1,5-a]pyridine-7-carboxylic acid (400 mg, 67.36%). MS: M/e 274 (M+H)⁺.

### Step 7:5-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrazolo[1,5-a]pyridine-7-carboxamide

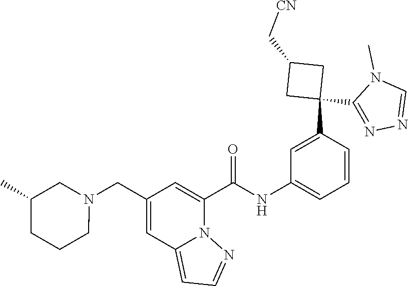 [SMILES: C[C@H]1CCCN(Cc2cc(C(=O)Nc3cccc([C@]4(c5nncn5C)C[C@@H](CC#N)C4)c3)n3nccc3c2)C1]

A solution of 5-{[(3S)-3-methylpiperidin-1-yl]methyl}pyrazolo[1,5-a]pyridine-7-carboxylic acid (28 mg, 0.100 mmol, 1.11 equiv, 97.3%), EDCI (27 mg, 0.134 mmol, 1.49 equiv, 95%), HOBT (19 mg, 0.134 mmol, 1.49 equiv, 95%) and TEA (0.06 mL, 0.450 mmol, 5 equiv, 98%) in DMF was stirred for 5 min at room temperature. To the mixture was added 2-[(1s, 3s)-3-(3-aminophenyl)-3-(4-methyl-1,2,4-triazol-3-yl)cyclobutyl]acetonitrile (30 mg, 0.090 mmol, 1.00 equiv, 80%) in DMF dropwise at room temperature. The resulting mixture was stirred for additional overnight at 40° C. The mixture was allowed to cool down to room temperature. The reaction mixture was purified by reverse flash chromatography with the following conditions: column, C18 silica gel; mobile phase, MeCN in Water (0.1% TFA), 45% to 50% gradient in 2 min; detector, UV 220 nm. The crude product was purified by Prep-HPLC with the following conditions (2 #SHIMADZU (HPLC-01)): Column, YMC-Actus Triart C18 ExRS, 30*150 mm, 5 μm; mobile phase, Water (10 mmol/L NH4HCO3+0.1% NH3·H2O) and ACN (42% ACN up to 72% in 8 min); This resulted in 5-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrazolo[1,5-a]pyridine-7-carboxamide (12.9 mg, 27.16%). MS: M/e 523 (M+H)⁺; ¹H NMR (400 MHz, DMSO-d₆) δ 12.49 (s, 1H), 8.33 (s, 1H), 8.25 (d, J=2.4 Hz, 1H), 7.86 (s, 1H), 7.81-7.73 (m, 2H), 7.73-7.65 (m, 1H), 7.45 (t, J=7.9 Hz, 1H), 7.21 (d, J=9.0 Hz, 1H), 6.85 (d, J=2.5 Hz, 1H), 3.55 (s, 2H), 3.22 (s, 3H), 2.94-2.68 (m, 9H), 1.94 (t, J=11.0 Hz, 1H), 1.75-1.39 (m, 5H), 1.73-1.41 (m, 4H) ppm.

### Compound A12: 3-fluoro-6-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}imidazo[1,2-a]pyridine-8-carboxamide

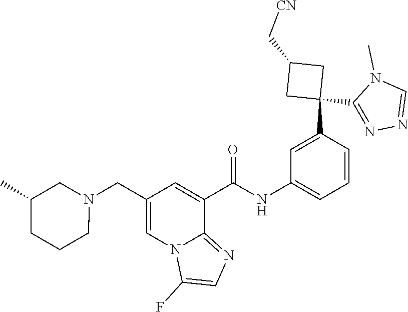 [SMILES: C[C@H]1CCCN(Cc2cc(C(=O)Nc3cccc([C@]4(c5nncn5C)C[C@@H](CC#N)C4)c3)c3ncc(F)n3c2)C1]

To a stirred mixture of 3-fluoro-6-{[(3S)-3-methylpiperidin-1-yl]methyl}imidazo[1,2-a]pyridine-8-carboxylic acid (23 mg, 0.073 mmol, 1.11 equiv) and HOBT (15 mg, 0.105 mmol, 1.60 equiv), EDCI (20 mg, 0.099 mmol, 1.51 equiv), Et3N (29 mg, 0.272 mmol, 4.14 equiv,) in DMF stirred for 5 mins was added 2-[(1s, 3s)-3-(3-aminophenyl)-3-(4-methyl-1,2,4-triazol-3-yl)cyclobutyl]acetonitrile (22 mg, 0.066 mmol, 1.00 equiv) dropwise at room temperature. The resulting mixture was stirred for 14 h at 40° C. The residue was purified by reverse flash chromatography with the following conditions: column, C18 silica gel; mobile phase, MeCN in Water (0.1% NH3·H2O), 0% to 95% gradient in 15 min; detector, UV 254 nm. The crude product (55 mg) was purified by Prep-HPLC with the following conditions (2 #SHIMADZU (HPLC-01)): Column, XBridge Prep OBD C18 Column, 30*150 mm, 5 μm; mobile phase, Water (10 mmol/L NH4HCO3+0.1% NH3·H2O) and ACN (40% ACN up to 70% in 68 min); This resulted in 3-fluoro-6-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}imidazo[1,2-a]pyridine-8-carboxamide (21.6 mg, 58.56%). MS: M/e 541 (M+H)⁺; ¹H NMR (400 MHz, DMSO-d₆) δ 11.29 (s, 1H), 8.68 (d, J=1.6 Hz, 1H), 8.33 (s, 1H), 8.09 (d, J=1.6 Hz, 1H), 7.93 (d, J=7.6 Hz, 1H), 7.77 (s, 1H), 7.63 (d, J=8.1 Hz, 1H), 7.50-7.39 (m, 1H), 7.20 (d, J=7.7 Hz, 1H), 3.52 (s, 2H), 3.22 (s, 3H), 2.95-2.66 (m, 9H), 1.98-1.85 (m, 1H), 1.72-1.41 (m, 5H), 0.94-0.77 (m, 4H) ppm.

### Compound A13: N-(3-((1s,3R)-3-(cyanomethyl)-1-(4-methyl-4H-1,2,4-triazol-3-yl)cyclobutyl)phenyl)-2-fluoro-6-(((S)-3-methylpiperidin-1-yl)methyl)imidazo[1,2-a]pyridine-8-carboxamide

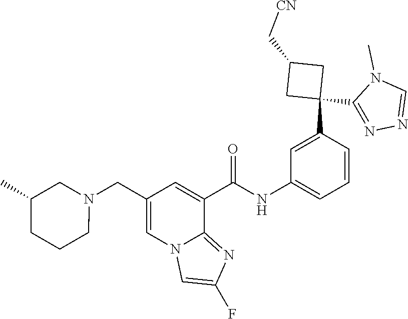 [SMILES: C[C@H]1CCCN(Cc2cc(C(=O)Nc3cccc([C@]4(c5nncn5C)C[C@@H](CC#N)C4)c3)c3nc(F)cn3c2)C1]

To a stirred mixture of (S)-2-fluoro-6-((3-methylpiperidin-1-yl)methyl)imidazo[1,2-a]pyridine-8-carboxylic acid (22 mg, 0.065 mmol, 1.09 equiv, 86.5%) and HOBT (13 mg, 0.091 mmol, 1.53 equiv, 95%), EDCI (19 mg, 0.094 mmol, 1.57 equiv, 95%), Et3N (26 mg, 0.244 mmol, 4.08 equiv, 95%) in DMF stirred for 5 mins were added 2-[(1s, 3s)-3-(3-aminophenyl)-3-(4-methyl-1,2,4-triazol-3-yl)cyclobutyl]acetonitrile (20 mg, 0.060 mmol, 1.00 equiv, 80%) dropwise at room temperature. The resulting mixture was stirred for 12 h at room temperature. The residue was purified by reverse flash chromatography with the following conditions: column, C18 silica gel; mobile phase, MeCN in Water (0.1% NH3·H2O), 0% to 95% gradient in 15 min; detector, UV 254 nm. The crude product (30 mg) was purified by Chiral-Prep-HPLC with the following conditions (2 #SHIMADZU (HPLC-01)): Column, YMC-Actus Triart C18 ExRS, 30*150 mm, 5 μm; mobile phase, Water (10 mmol/L NH4HCO3+0.1% NH3·H2O) and ACN (42% ACN up to 72% in 8 min); This resulted in N-(3-((1s,3R)-3-(cyanomethyl)-1-(4-methyl-4H-1,2,4-triazol-3-yl)cyclobutyl)phenyl)-2-fluoro-6-(((S)-3-methylpiperidin-1-yl)methyl)imidazo[1,2-a]pyridine-8-carboxamide (7.6 mg, 23.30%). MS: M/e 541 (M+H)*; ¹HNMR (400 MHz, DMSO-d₆) δ 11.29 (s, 1H), 8.68 (d, J=1.6 Hz, 1H), 8.33 (s, 1H), 8.09 (d, J=1.6 Hz, 1H), 7.93 (d, J=7.6 Hz, 1H), 7.77 (s, 1H), 7.63 (d, J=8.1 Hz, 1H), 7.50-7.39 (m, 1H), 7.20 (d, J=7.7 Hz, 1H), 3.52 (s, 2H), 3.22 (s, 3H), 2.95-2.66 (m, 9H), 1.98-1.85 (m, 1H), 1.72-1.41 (m, 5H), 0.94-0.77 (m, 4H) ppm.

### Compound A14: N-(3-((1s,3R)-3-(cyanomethyl)-1-(4-methyl-4H-1,2,4-triazol-3-yl)cyclobutyl)phenyl)-7-(((S)-3-methylpiperidin-1-yl)methyl)-[1,2,4]triazolo[1,5-a]pyridine-5-carboxamide

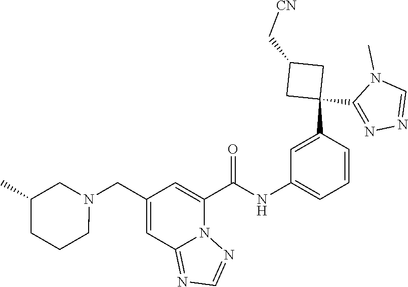 [SMILES: C[C@H]1CCCN(Cc2cc(C(=O)Nc3cccc([C@]4(c5nncn5C)C[C@@H](CC#N)C4)c3)n3ncnc3c2)C1]

### Step A: methyl 7-bromo-[1,2,4]triazolo[1,5-a]pyridine-5-carboxylate

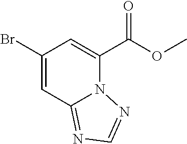

To a solution of methyl 6-amino-4-bromopicolinate (200 mg, 0.87 mmol) in MeOH (4 mL) was added DMF-DMA (0.23 mL, 1.73 mmol). The reaction mixture was stirred for 12 h at 70° C. The reaction was cooled down to room temperature, and Hydroxylamine hydrochloride (120 mg, 1.73 mmol) was added. The reaction mixture was stirred for 2 h at 25° C. The solvent was removed by in vacuo. The residue was dissolved in THF (5 mL) and TFAA (0.36 mL, 2.60 mmol) was added dropwise at 0° C. The reaction mixture was stirred for 12 h at 25° C. After completed, the reaction was quenched with aq NaHCO₃ (30 mL) and extracted with DCM/MeOH (10/1, 3×50 mL), dried over Na₂SO₄ and concentrated under vacuum. The residue was purified by Prep-HPLC to afford the title compound (130 mg, 58%). ¹H NMR (400 MHz, CDCl₃-d) δ 8.48 (s, 1H), 8.17 (d, J=2.0 Hz, 1H), 7.88 (d, J=2.0 Hz, 1H), 4.10 (s, 3H). MS: M/e 256, 258 (M+H)⁺.

### Step B: 7-bromo-[1,2,4]triazolo[1,5-a]pyridine-5-carboxylic acid

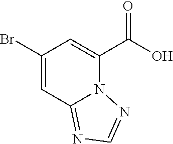 [SMILES: O=C(O)c1cc(Br)cc2ncnn12]

To a solution of methyl 7-bromo-[1,2,4]triazolo[1,5-a]pyridine-5-carboxylate (50 mg, 0.195 mmol) in MeOH (5 mL)/H₂O (0.5 mL) was added LiOH H₂O (41 mg, 0.976 mmol). The reaction mixture was stirred for 12 h at 25° C. After completed, the solvent was concentrated under vacuum. The residue was diluted with water and neutralized with 2 N HCl and freed dried to afford the title compound (50 mg, crude). MS: M/e 242, 244 (M+H)*.

### Step C: 7-bromo-N-(3-((1s, 3s)-3-(cyanomethyl)-1-(4-methyl-4H-1,2,4-triazol-3-yl)cyclobutyl)phenyl)-[1,2,4]triazolo[1,5-a]pyridine-5-carboxamide

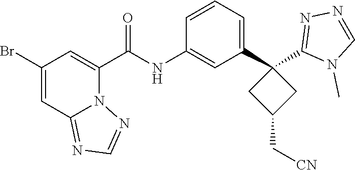 [SMILES: Cn1cnnc1[C@]1(c2cccc(NC(=O)c3cc(Br)cc4ncnn34)c2)C[C@H](CC#N)C1]

To a solution of 7-bromo-[1,2,4]triazolo[1,5-a]pyridine-5-carboxylic acid (50 mg, crude), 2-((1s, 3s)-3-(3-aminophenyl)-3-(4-methyl-4H-1,2,4-triazol-3-yl)cyclobutyl) acetonitrile (52 mg, 0.195 mmol), DIEA (50 mg, 0.387 mmol) in DMF (2 mL) was added HATU (110 mg, 0.289 mmol). The reaction mixture was stirred for 1 h at 25° C. After completed, the reaction was quenched with aq NH₄Cl (10 mL) and extracted with DCM (3×30 mL), dried over Na₂SO₄ and concentrated under vacuum. The residue was purified by Prep-TLC (DCM/MeOH=15/1) to afford the title compound (80 mg, 83%). MS: M/e 491, 493 (M+H)⁺.

### Step D: N-(3-((1s,3R)-3-(cyanomethyl)-1-(4-methyl-4H-1,2,4-triazol-3-yl)cyclobutyl) phenyl)-7-(((S)-3-methylpiperidin-1-yl)methyl)-[1,2,4]triazolo[1,5-a]pyridine-5-carboxamide

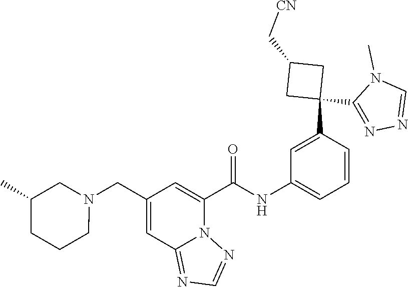 [SMILES: C[C@H]1CCCN(Cc2cc(C(=O)Nc3cccc([C@]4(c5nncn5C)C[C@@H](CC#N)C4)c3)n3ncnc3c2)C1]

To a solution of 7-bromo-N-(3-((1s, 3s)-3-(cyanomethyl)-1-(4-methyl-4H-1,2,4-triazol-3-yl)cyclobutyl)phenyl)-[1,2,4]triazolo[1,5-a]pyridine-5-carboxamide (80 mg, 0.163 mmol), potassium (S)-trifluoro((3-methylpiperidin-1-yl)methyl)borate (55 mg, 0.252 mmol) in THF/H₂O (2 mL)/H₂O (0.2 mL) were added Pd(OAc)₂ (4 mg, 0.017 mmol) and Xphos (16 mg, 0.034 mmol), Cs₂CO₃ (106 mg, 0.325 mmol) under N₂. The reaction mixture was stirred for 12 h at 100° C. After completed, the reaction was quenched with water (10 mL) and extracted with EtOAc (3×20 mL), dried over Na₂SO₄ and concentrated under vacuum. The residue was purified by prep-HPLC to afford the title compound (25 mg, 29%). ¹H NMR (400 MHz, CD₃OD) δ 8.64 (s, 1H), 8.33 (s, 1H), 8.17 (s, 1H), 7.97 (d, J=6.0 Hz, 2H), 7.70 (d, J=8.0 Hz, 1H), 7.48 (t, J=8.0 Hz, 1H), 7.25 (d, J=8.0 Hz, 1H), 3.72 (s, 2H), 3.34 (s, 3H), 3.08-3.00 (m, 2H), 2.99-2.78 (m, 5H), 2.67 (d, J=5.7 Hz, 2H), 2.05 (t, J=9.6 Hz, 1H), 1.80-1.57 (m, 5H), 1.00-0.90 (m, 1H), 0.88 (d, J=5.2 Hz, 3H) ppm. MS: M/e 524 (M+H)⁺.

### Compound A15: N-(3-((1s, 3s)-3-(cyanomethyl)-1-(4-methyl-4H-1,2,4-triazol-3-yl)cyclobutyl)phenyl)-6-((((1-methylcyclopentyl)methyl)amino)methyl)imidazo[1,2-a]pyridine-8-carboxamide

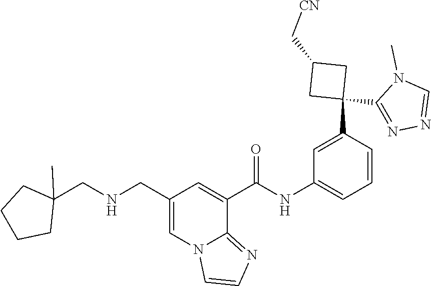 [SMILES: Cn1cnnc1[C@]1(c2cccc(NC(=O)c3cc(CNCC4(C)CCCC4)cn4ccnc34)c2)C[C@H](CC#N)C1]

### Step 1: methyl 6-((((1-methylcyclopentyl)methyl)amino)methyl)imidazo[1,2-a]pyridine-8-carboxylate

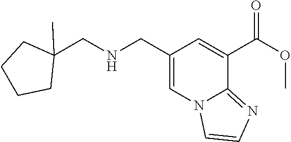 [SMILES: COC(=O)c1cc(CNCC2(C)CCCC2)cn2ccnc12]

To a mixture of methyl 6-formylimidazo[1,2-a]pyridine-8-carboxylate (227.38 mg, 1.11 mmol) and (1-methylcyclopentyl)methanamine hydrochloride (250 mg, 1.67 mmol) in DCM (3 mL) was added TEA (169.04 mg, 1.67 mmol) and NaBH(OAc)₃ (472.04 mg, 2.23 mmol) in one portion at 20° C. The mixture was stirred at 20° C. for 12 h. The mixture was poured into water (5 mL) and extracted with DCM (15 mL). The combined organic phase was washed with brine (5 mL), dried over Na₂SO₄, filtered and concentrated to give the residue, which was purified by column chromatography (SiO₂, Petroleum ether/Ethyl acetate=100/1 to 0/1) to give the product (245 mg, 73% yield). MS: M/e 302 (M+1)+

### Step 2: methyl 6-(((tert-butoxycarbonyl)((1-methylcyclopentyl)methyl)amino)methyl)imidazo[1,2-a]pyridine-8-carboxylate

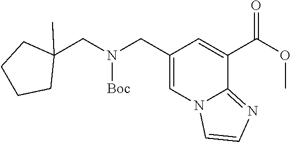 [SMILES: COC(=O)c1cc(CN(CC2(C)CCCC2)C(=O)OC(C)(C)C)cn2ccnc12]

To a solution of methyl 6-((((1-methylcyclopentyl)methyl)amino)methyl)imidazo[1,2-a]pyridine-8-carboxylate (245 mg, 812.90 mol) and (Boc)₂O (354.83 mg, 1.63 mmol) in DCM (3 mL) was added TEA (246.78 mg, 2.44 mmol). The mixture was stirred for 2 hr at rt. The mixture was poured into water (5 mL) and extracted with DCM (10 mL, 5 mL). The combined organic phase was washed with brine (5 mL), dried over Na₂SO₄, filtered and concentrated to give the residue, which was purified by column chromatography (SiO₂, Petroleum ether/Ethyl acetate=100/1 to 0/1) to give the product (290 mg, 88.85% yield). MS: M/e 402 (M+1)⁺.

### Step 3: 6-(((tert-butoxycarbonyl)((1-methylcyclopentyl)methyl)amino)methyl)imidazo[1,2-a]pyridine-8-carboxylic acid

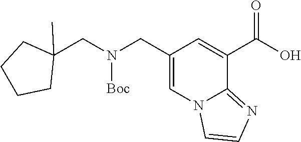 [SMILES: CC1(CN(Cc2cc(C(=O)O)c3nccn3c2)C(=O)OC(C)(C)C)CCCC1]

To a solution of methyl 6-(((tert-butoxycarbonyl)((1-methylcyclopentyl)methyl)amino)methyl)imidazo[1,2-a]pyridine-8-carboxylate (290 mg, 722 mol) in MeOH/THF/H₂O (1 mL/1 mL/1 mL) was added LiOH·H₂O (36.37 mg, 867 mol). The mixture was stirred for 12 hr at rt. The mixture was filtered and concentrated to give the crude product (280 mg, crude), which was used to the next step without purification. MS: M/e 388 (M+1)⁺.

### Step 4: tert-butyl ((8-((3-((1s, 3s)-3-(cyanomethyl)-1-(4-methyl-4H-1,2,4-triazol-3-yl)cyclobutyl)phenyl)carbamoyl)imidazo[1,2-a]pyridin-6-yl)methyl)((1-methylcyclopentyl)methyl)carbamate

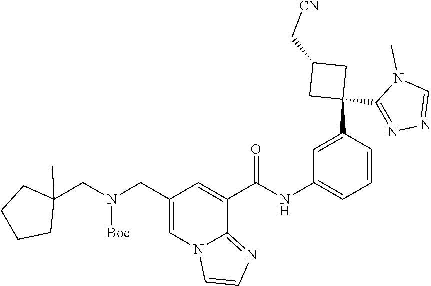 [SMILES: Cn1cnnc1[C@]1(c2cccc(NC(=O)c3cc(CN(CC4(C)CCCC4)C(=O)OC(C)(C)C)cn4ccnc34)c2)C[C@H](CC#N)C1]

To a solution of 6-(((tert-butoxycarbonyl)((1-methylcyclopentyl)methyl)amino)methyl)imidazo[1,2-a]pyridine-8-carboxylic acid (200 mg, 438.74 mol) in DMF (2 mL) was added DIEA (113.41 mg, 877.48 mol) and HATU (200.19 mg, 526.49 mol). The mixture was stirred for 5 mins at rt, then 2-((1s, 3s)-3-(3-aminophenyl)-3-(4-methyl-4H-1,2,4-triazol-3-yl)cyclobutyl)acetonitrile (117.29 mg, 438.74 μmol) was added. The mixture was stirred for 12 hrs at rt. The mixture was poured into water (2 mL) and extracted with DCM (4 mL, 2 mL). The combined organic phase was filtered and concentrated to give the residue, which was purified by prep-HPLC (column=Waters Xbridge Prep OBD C18 (150*40 mm*10 um); mobile phase=water (NH4HCO3)-ACN, B %=25%-55%; 8 min) to give the product (150 mg, 53.69% yield). MS: M/e 637 (M+1)⁺.

### Step 5: N-(3-((1s, 3s)-3-(cyanomethyl)-1-(4-methyl-4H-1,2,4-triazol-3-yl)cyclobutyl)phenyl)-6-((((1-methylcyclopentyl)methyl)amino)methyl)imidazo[1,2-a]pyridine-8-carboxamide

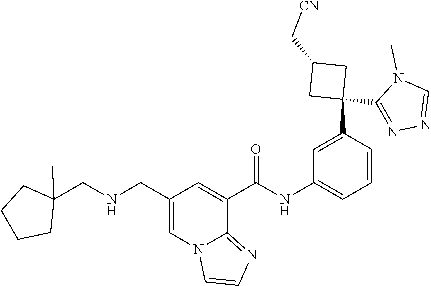 [SMILES: Cn1cnnc1[C@]1(c2cccc(NC(=O)c3cc(CNCC4(C)CCCC4)cn4ccnc34)c2)C[C@H](CC#N)C1]

To a solution of tert-butyl ((8-((3-((1s, 3s)-3-(cyanomethyl)-1-(4-methyl-4H-1,2,4-triazol-3-yl)cyclobutyl)phenyl)carbamoyl)imidazo[1,2-a]pyridin-6-yl)methyl)((1-methylcyclopentyl)methyl)carbamate (70 mg, 109.9 μmol) in DCM (1 mL) was added TMSI (109.98 mg, 549.63 μmol) at 0° C. The mixture was poured into water (2 mL) and extracted with DCM (6 mL). The combined organic phase was filtered and concentrated to give the residue, which was purified by prep-HPLC (column=Waters Xbridge Prep OBD C18 (150*40 mm*0 um); mobile phase=water (NH4HCO3)-ACN, B %=25%-55%; 8 min) to give the product (15.73 mg, 26.7% yield). MS: M/e 537 (M+1)+; ¹H NMR (400 MHz, DMSO-d₆) δ=12.43 (s, 1H), 8.71 (s, 1H), 8.32 (s, 1H), 8.19 (s, 1H), 8.16-8.13 (m, 1H), 7.79 (s, 1H), 7.76-7.73 (m, 1H), 7.72-7.67 (m, 1H), 7.44 (t, J=8.0 Hz, 1H), 7.20 (br d, J=7.6 Hz, 1H), 3.79 (s, 2H), 3.22 (s, 3H), 2.89 (br s, 2H), 2.76 (d, J=6.4 Hz, 3H), 2.73-2.69 (m, 2H), 2.35 (s, 2H), 2.17 (br s, 1H), 1.60-1.43 (m, 6H), 1.28-1.19 (m, 2H), 0.96 (s, 3H).

### Compound A16: N-(3-((1s, 3s)-3-methyl-1-(4-methyl-4H-1,2,4-triazol-3-yl)cyclobutyl)phenyl)-6-(((1-methylcyclopropyl)amino)methyl)imidazo[1,2-a]pyridine-8-carboxamide

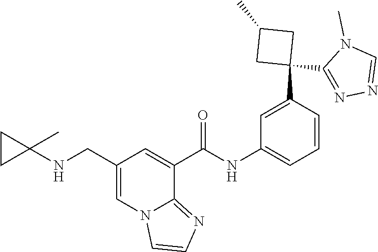 [SMILES: Cn1cnnc1[C@]1(c2cccc(NC(=O)c3cc(CNC4(C)CC4)cn4ccnc34)c2)C[C@H](C)C1]

### Step 1: methyl 6-(((1-methylcyclopropyl)amino)methyl)imidazo[1,2-a]pyridine-8-carboxylate

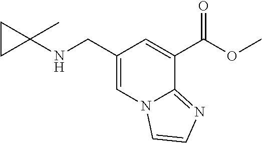 [SMILES: COC(=O)c1cc(CNC2(C)CC2)cn2ccnc12]

To a solution of 1-methylcyclopropan-1-amine hydrochloride (1.58 g, 14.69 mmol) in DCE (6 mL) was added TEA (743.37 mg, 7.35 mmol), AcOH (882.33 mg, 14.69 mmol) and methyl 6-formylimidazo[1,2-a]pyridine-8-carboxylate (1 g, 4.90 mmol). The mixture was stirred at 25° C. for 5 min. Then NaBH(OAc)₃ (3.11 g, 14.69 mmol) was added at 25° C. The resulting mixture was stirred at 25° C. for 16 hr. The aqueous phase was adjusted to pH 9-10 with saturated aqueous Na2CO3, and then extracted with EtOAc 30 mL (10 mL*3). The combined organic layers were washed with brine 30 mL (10 mL*3), dried over Na2SO4, filtered and concentrated under reduced pressure to give a residue. The residue was purified by column chromatography (SiO2, Petroleum ether/Ethyl acetate=100/1 to 0/1) to give the product (520 mg, 37.67% yield) was obtained. MS: M/e 260 (M+1)⁺. ¹H NMR (400 MHz, DMSO-d₆) δ=8.68 (s, 1H), 8.01 (s, 1H), 7.82 (s, 1H), 7.60 (s, 1H), 3.89 (s, 3H), 3.76 (s, 2H), 1.25 (s, 3H), 0.50 (br s, 2H), 0.31 (br s, 2H) ppm.

### Step 2: lithium 6-(((1-methylcyclopropyl)amino)methyl)imidazo[1,2-a]pyridine-8-carboxylate

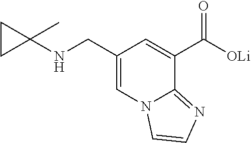

To a solution of methyl 6-(((1-methylcyclopropyl)amino)methyl)imidazo[1,2-a]pyridine-8-carboxylate (280 mg, 1.08 mmol) in H2O (1 mL), THF (1 mL), MeOH (1 mL) was added LiOH·H2O (54.38 mg, 1.30 mmol). The mixture was stirred at 40° C. for 1 hr. The reaction mixture was concentrated under reduced pressure to give a product (150 mg, crude). MS: M/e 246 (M+1)⁺.

### Step 3: N-(3-((1s, 3s)-3-methyl-1-(4-methyl-4H-1,2,4-triazol-3-yl)cyclobutyl)phenyl)-6-(((1-methylcyclopropyl)amino)methyl)imidazo[1,2-a]pyridine-8-carboxamide

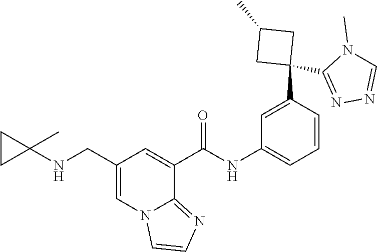 [SMILES: Cn1cnnc1[C@]1(c2cccc(NC(=O)c3cc(CNC4(C)CC4)cn4ccnc34)c2)C[C@H](C)C1]

To a stirred solution of 3-((1s, 3s)-3-methyl-1-(4-methyl-4H-1,2,4-triazol-3-yl)cyclobutyl)aniline (151.93 mg, 626.97 umol) and lithium 6-(((1-methylcyclopropyl)amino)methyl)imidazo[1,2-a]pyridine-8-carboxylate (210 mg, 626.97 umol) in PYRIDINE (5 mL) was added T3P (1.20 g, 1.88 mmol, 50 wt. % in ethyl acetate) at 25° C. The resulting mixture was stirred for 3 h at 25° C. The reaction mixture was quenched by addition H2O 10 mL at 20° C., and then extracted with EtOAc 30 mL (10 mL*3). The combined organic layers were washed with brine 30 mL (10 mL*3), dried over Na2SO4, filtered and concentrated under reduced pressure to give a residue, which was purified by prep-HPLC (column: Waters Xbridge BEH C18 100*30 mm*10 um; mobile phase: [water (NH4HCO3)-ACN]; B %: 30%-60%, 8 min) to give the product (182.57 mg, 31.01% yield). MS: M/e 470 (M+1)⁺. ¹H NMR (400 MHz, DMSO-d₆) δ=12.40 (br s, 1H), 8.70 (br s, 1H), 8.30 (br s, 1H), 8.11 (br d, J=8.0 Hz, 2H), 7.75 (br d, J=8.0 Hz, 2H), 7.66 (br d, J=4.0 Hz, 1H), 7.41 (br s, 1H), 7.15 (br s, 1H), 3.81 (br s, 2H), 3.19 (br s, 3H), 2.83 (br s, 2H), 2.54 (br s, 3H), 1.26 (br s, 3H), 1.08 (br s, 3H), 0.51 (br s, 2H), 0.32 (br s, 2H) ppm.

### Compound A17: N-(3-((1s, 3s)-3-(cyanomethyl)-1-(4-methyl-4H-1,2,4-triazol-3-yl)cyclobutyl)phenyl)-3-fluoro-6-(((1-methylcyclopropyl)amino)methyl)imidazo[1,2-a]pyridine-8-carboxamide

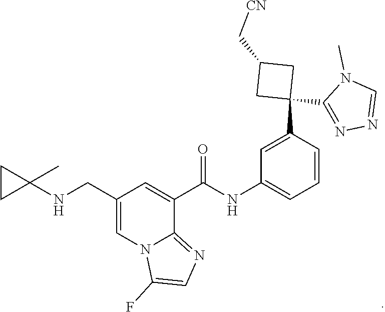 [SMILES: Cn1cnnc1[C@]1(c2cccc(NC(=O)c3cc(CNC4(C)CC4)cn4c(F)cnc34)c2)C[C@H](CC#N)C1]

### Step 1: N-(3-((1s, 3s)-3-(cyanomethyl)-1-(4-methyl-4H-1,2,4-triazol-3-yl)cyclobutyl)phenyl)-3-fluoro-6-(hydroxymethyl)imidazo[1,2-a]pyridine-8-carboxamide

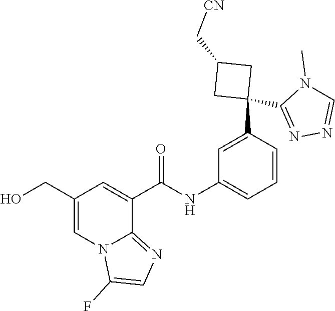 [SMILES: Cn1cnnc1[C@]1(c2cccc(NC(=O)c3cc(CO)cn4c(F)cnc34)c2)C[C@H](CC#N)C1]

A mixture of lithium 3-fluoro-6-(hydroxymethyl)imidazo[1,2-a]pyridine-8-carboxylate (0.2 g, 740.42 umol), 2-((1s, 3s)-3-(3-aminophenyl)-3-(4-methyl-4H-1,2,4-triazol-3-yl)cyclobutyl)acetonitrile (197.93 mg, 740.42 umol), HATU (337.84 mg, 888.50 umol) DIEA (287.08 mg, 2.22 mmol) DMF (4 mL) was stirred at 25° C. for 2 h. The mixture was poured into 50 ml H2O, extracted with Dichloromethane (30 mL*2), washed with brine(20 mL*2), the combined organic layers were dried over Na2SO4, filtered and concentrated under reduced pressure to give a residue, which was purified by prep-HPLC (column: Waters Xbridge BEH C18 100*30 mm*10 um; mobile phase: [water (NH4HCO3)-ACN]; B %: 20%-50%, 8 min) to give the product (0.045 g 13.23% yield). MS: M/e 460 (M+1)⁺. ¹H NMR (400 MHz, DMSO-d₆) δ=11.94 (s, 1H), 8.45 (d, J=1.2 Hz, 1H), 8.32 (s, 1H), 8.13 (d, J=1.6 Hz, 1H), 7.69 (d, J=1.2 Hz, 1H), 7.63 (d, J=8.0 Hz, 1H), 7.46-7.42 (m, 1H), 7.22 (br d, J=8.0 Hz, 1H), 5.59 (t, J=4.0 Hz, 1H), 4.63 (d, J=4.0 Hz, 2H), 3.22 (s, 3H), 2.91-2.85 (m, 2H), 2.80-2.65 (m, 6H).

### Step 2: 6-(chloromethyl)-N-(3-((1s, 3s)-3-(cyanomethyl)-1-(4-methyl-4H-1,2,4-triazol-3-yl)cyclobutyl)phenyl)-3-fluoroimidazo[1,2-a]pyridine-8-carboxamide & (8-((3-((1s, 3s)-3-(cyanomethyl)-1-(4-methyl-4H-1,2,4-triazol-3-yl)cyclobutyl)phenyl)carbamoyl)-3-fluoroimidazo[1,2-a]pyridin-6-yl)methyl methanesulfonate

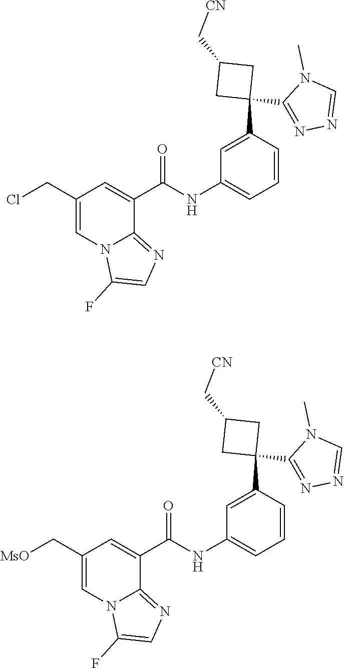 [SMILES: Cn1cnnc1[C@]1(c2cccc(NC(=O)c3cc(CCl)cn4c(F)cnc34)c2)C[C@H](CC#N)C1 ; Cn1cnnc1[C@]1(c2cccc(NC(=O)c3cc(COS(C)(=O)=O)cn4c(F)cnc34)c2)C[C@H](CC#N)C1]

In a mixture of N-(3-((1s, 3s)-3-(cyanomethyl)-1-(4-methyl-4H-1,2,4-triazol-3-yl)cyclobutyl)phenyl)-3-fluoro-6-(hydroxymethyl)imidazo[1,2-a]pyridine-8-carboxamide (60 mg, 130.58 umol) and TEA (105.71 mg, 1.04 mmol), DMF (2 mL) was added MsCl (74.79 mg, 652.92 umol) at 0° C. The resulting mixture was stirred at 25° C. for 12 hrs. The mixture was poured into 5 ml H₂O, extracted with Dichloromethane (5 mL*5), washed with brine(5 mL*2), the combined organic layers were dried over Na₂SO₄, filtered and concentrated under reduced pressure to give the 6-(chloromethyl)-N-(3-((1s, 3s)-3-(cyanomethyl)-1-(4-methyl-4H-1,2,4-triazol-3-yl)cyclobutyl)phenyl)-3-fluoroimidazo[1,2-a]pyridine-8-carboxamide and (8-((3-((1s, 3s)-3-(cyanomethyl)-1-(4-methyl-4H-1,2,4-triazol-3-yl)cyclobutyl)phenyl)carbamoyl)-3-fluoroimidazo[1,2-a]pyridin-6-yl)methyl methanesulfonate (60 mg, crude), which was used to the next step without purification. MS: M/e 478 (M+1)+

### Step 3: N-(3-((1s, 3s)-3-(cyanomethyl)-1-(4-methyl-4H-1,2,4-triazol-3-yl)cyclobutyl)phenyl)-3-fluoro-6-(((1-methylcyclopropyl)amino)methyl)imidazo[1,2-a]pyridine-8-carboxamide

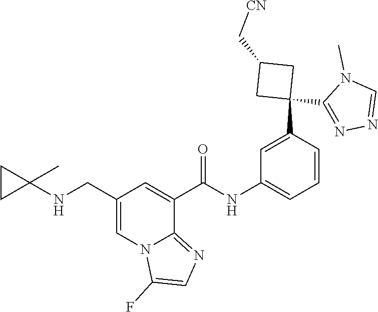 [SMILES: Cn1cnnc1[C@]1(c2cccc(NC(=O)c3cc(CNC4(C)CC4)cn4c(F)cnc34)c2)C[C@H](CC#N)C1]

To a solution of 1-methylcyclopropan-1-amine hydrochloride (54.02 mg, 502.18 umol) in DMF (2 mL) was added TEA (76.22 mg, 753.26 umol). The mixture was stirred at 25° C. for 5 min. Then pyridine, the product of step 2 (60 mg) and Cs₂CO₃ (81.81 mg, 251.09 umol) was added at 25° C. The resulting mixture was stirred at 25° C. for 16 hrs. The mixture was filtered, the filtrate was concentrated in vacuo, which was purified by Prep-HPLC (column: Waters Xbridge Prep OBD C18 150*40 mm*10 um; mobile phase: [water (NH₄HCO₃)-ACN]; B %: 30%-50%, 8 min) to give the product (13.53 mg, 21.03% yield). MS: M/e 513 (M+1)⁺. ¹H NMR (400 MHz, DMSO-d₆) δ=11.94 (s, 1H), 8.45 (s, 1H), 8.32 (s, 1H), 8.11 (d, J=1.6 Hz, 1H), 7.73 (s, 1H), 7.68 (br d, J=8.0 Hz, 1H), 7.61 (d, J=8.0 Hz, 1H), 7.44 (t, J=8.0 Hz, 1H), 7.20 (br d, J=8.0 Hz, 1H), 3.85 (s, 2H), 3.21 (s, 3H), 2.94-2.82 (m, 2H), 2.82-2.66 (m, 5H), 2.55 (br d, J=4.0 Hz, 1H), 1.27 (s, 3H), 0.59-0.46 (m, 2H), 0.38-0.27 (m, 2H) ppm.

### Compound A18: 6-((isobutylamino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

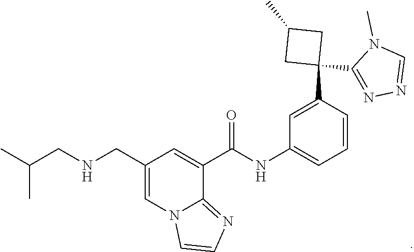 [SMILES: CC(C)CNCc1cc(C(=O)Nc2cccc([C@]3(c4nncn4C)C[C@@H](C)C3)c2)c2nccn2c1]

### Step 1: methyl 6-((isobutylamino)methyl)imidazo[1,2-a]pyridine-8-carboxylate

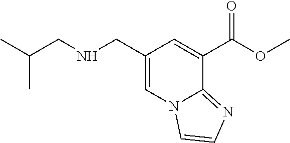 [SMILES: COC(=O)c1cc(CNCC(C)C)cn2ccnc12]

To a solution of 2-methylpropan-1-amine (214.92 mg, 2.94 mmol) and methyl 6-formylimidazo[1,2-a]pyridine-8-carboxylate (200 mg, 979.52 umol) in DCE (3 mL) was added NaBH(OAc)₃ (622.80 mg, 2.94 mmol) and CH3COOH (176.46 mg, 2.94 mmol). The mixture was stirred at 25° C. for 16 hr. Then NaBH(OAc)₃ (415.20 mg, 1.96 mmol) and CH3COOH (58.82 mg, 979.52 umol) was added dropwise at 25° C. The resulting mixture was stirred at 25° C. for 12 hr. The aqueous phase was adjusted to pH 9-10 with saturated aqueous Na2CO3, and then extracted with EtOAc 30 mL (10 mL*3). The combined organic layers were washed with brine 30 mL (10 mL*3), dried over Na2SO4, filtered and concentrated under reduced pressure to give a residue, which was purified by Prep-TLC (SiO2, Ethyl acetate:Methanol=4:1, R_{f}=0.5) to give the product (110 mg, 42.97% yield). MS: M/e 262 (M+1)⁺.

### Step 2: lithium 6-((isobutylamino)methyl)imidazo[1,2-a]pyridine-8-carboxylate

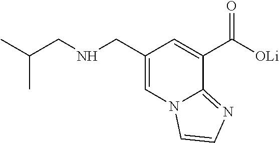

To a solution of methyl 6-((isobutylamino)methyl)imidazo[1,2-a]pyridine-8-carboxylate (110 mg, 420.94 umol) in H2O (1 mL), MeOH (1 mL) and THF (1 mL) was added LiOH·H2O (21.20 mg, 505.13 umol). The mixture was stirred at 25° C. for 2 hr. The mixture was filtered and concentrated to give the crude product (120 mg, crude), which was used to the next step without purification. MS: M/e 248 (M+1)⁺.

### Step 3: 6-((isobutylamino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

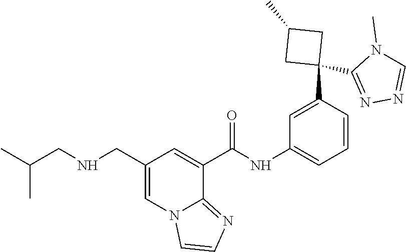 [SMILES: CC(C)CNCc1cc(C(=O)Nc2cccc([C@]3(c4nncn4C)C[C@@H](C)C3)c2)c2nccn2c1]

To a stirred solution of 3-((1s, 3s)-3-methyl-1-(4-methyl-4H-1,2,4-triazol-3-yl)cyclobutyl)aniline (91.87 mg, 379.11 umol) and lithium 6-((isobutylamino)methyl)imidazo[1,2-a]pyridine-8-carboxylate (120 mg, 379.11 umol) in PYRIDINE (5 mL) was added T3P (723.75 mg, 1.14 mmol, 50% purity) at 25° C. The resulting mixture was stirred for 3 h at 25° C. The reaction mixture was quenched by addition H2O 10 mL at 20° C., and then extracted with EtOAc 30 mL (10 mL*3). The combined organic layers were washed with brine 30 mL (10 mL*3), dried over Na2SO4, filtered and concentrated under reduced pressure to give a residue, which was purified by prep-HPLC (column: Waters Xbridge BEH C18 100*25 mm*5 um; mobile phase: [water (NH4HCO3)-ACN]; B %: 20%-50%, 8 min) to give the product (34.13 mg, 19.09% yield). MS: M/e 472 (M+1)⁺. ¹H NMR (400 MHz, DMSO-d₆) δ=12.41 (s, 1H), 8.72 (d, J=1.2 Hz, 1H), 8.30 (s, 1H), 8.16 (d, J=1.6 Hz, 1H), 8.14 (d, J=1.2 Hz, 1H), 7.78 (d, J=1.2 Hz, 1H), 7.74 (t, J=1.6 Hz, 1H), 7.67 (dd, J=1.2, 8.0 Hz, 1H), 7.42 (t, J=8.0 Hz, 1H), 7.19-7.12 (m, 1H), 3.76 (s, 2H), 3.20 (s, 3H), 2.84 (br d, J=4.0 Hz, 2H), 2.55 (br d, J=8.0 Hz, 3H), 2.32 (d, J=8.0 Hz, 2H), 1.69 (quind, J=8.0, 12.0 Hz, 1H), 1.09 (d, J=4.0 Hz, 3H), 0.87 (d, J=8.0 Hz, 6H);

### Compound A19: 6-((((S)-1-cyclopropylethyl)amino)methyl)-3-fluoro-N-(3-((1s,3R)-3-methyl-1-(4-methyl-4H-1,2,4-triazol-3-yl)cyclobutyl)phenyl)imidazo[1,2-a]pyridine-8-carboxamide

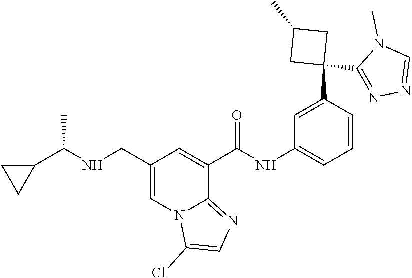 [SMILES: C[C@H](NCc1cc(C(=O)Nc2cccc([C@]3(c4nncn4C)C[C@@H](C)C3)c2)c2ncc(Cl)n2c1)C1CC1]

### Step 1: methyl 3-fluoro-6-(hydroxymethyl)imidazo[1,2-a]pyridine-8-carboxylate

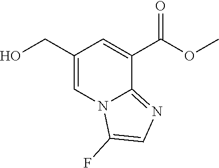 [SMILES: COC(=O)c1cc(CO)cn2c(F)cnc12]

To a solution of methyl 3-fluoro-6-formylimidazo[1,2-a]pyridine-8-carboxylate (2 g, 9.00 mmol) in EtOH (20 mL) was added NaBH4 (374.62 mg, 9.90 mmol) at 0° C. The mixture was stirred at 0-20° C. for 2 hr. The reaction mixture was quenched by addition H2O 20 mL at 25° C., and extracted with EA 60 mL (20 mL*3). The combined organic layers were washed with aq. NaCl 10 mL (5 mL*2), dried over Na2SO4, filtered and concentrated under reduced pressure to give a residue, which was purified by column chromatography (SiO2, Petroleum ether/Ethyl acetate/Dichloromethane=5/1/1 to 1/1/1) to give the product (800 mg, 33.69% yield). MS: M/e 225 (M+1)⁺.

### Step 2: lithium 3-fluoro-6-(hydroxymethyl)imidazo[1,2-a]pyridine-8-carboxylate

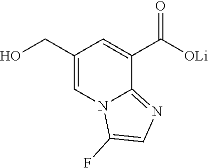

To a solution of methyl 3-fluoro-6-(hydroxymethyl)imidazo[1,2-a]pyridine-8-carboxylate (800 mg, 3.57 mmol) in THF (3 mL) and MeOH (3 mL) and H2O (3 mL) was added LiOH·H2O (179.69 mg, 4.28 mmol). The mixture was stirred at 20° C. for 2 hr. The yellow solid was re-crystallized from water, dried by hyophilization to give as a yellow solid (800 mg, crude), which was used to the next step without purification. MS: M/e 211 (M+1)⁺.

### Step 3: 3-fluoro-6-(hydroxymethyl)-N-(3-((1s, 3s)-3-methyl-1-(4-methyl-4H-1,2,4-triazol-3-yl)cyclobutyl)phenyl)imidazo[1,2-a]pyridine-8-carboxamide

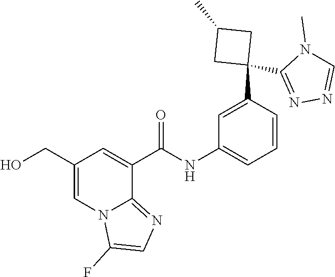 [SMILES: Cn1cnnc1[C@]1(c2cccc(NC(=O)c3cc(CO)cn4c(F)cnc34)c2)C[C@H](C)C1]

To a solution of lithium 3-fluoro-6-(hydroxymethyl)imidazo[1,2-a]pyridine-8-carboxylate (1.2 g, 5.00 mmol, 90% purity) and 3-((1s, 3s)-3-methyl-1-(4-methyl-4H-1,2,4-triazol-3-yl)cyclobutyl)aniline (1.21 g, 5.00 mmol) in DMF (15 mL) was added HATU (2.09 g, 5.50 mmol) and DIEA (1.29 g, 10.00 mmol). The mixture was stirred at 25° C. for 12 hr. The reaction mixture was quenched by addition H₂O 10 mL at 25° C., and then diluted with EtOAc 20 mL and extracted with EtOAc (30 mL*3). The combined organic layers were washed with saturated brine 2 mL, dried over anhydrous Na2SO4, filtered and concentrated under reduced pressure to give a residue, which was purified by prep-HPLC (column: Waters Xbridge BEH C18 250*70 mm*10 um; mobile phase: [water (NH4HCO3)-ACN]; B %: 25%-55%, 18 min) to give the product (0.333 g, 15.34% yield). MS: M/e 435 (M+1).

### Step 4: (3-fluoro-8-((3-((1s, 3s)-3-methyl-1-(4-methyl-4H-1,2,4-triazol-3-yl)cyclobutyl)phenyl)carbamoyl)imidazo[1,2-a]pyridin-6-yl)methyl methanesulfonate & 6-(chloromethyl)-3-fluoro-N-(3-((1s, 3s)-3-methyl-1-(4-methyl-4H-1,2,4-triazol-3-yl)cyclobutyl)phenyl)imidazo[1,2-a]pyridine-8-carboxamide

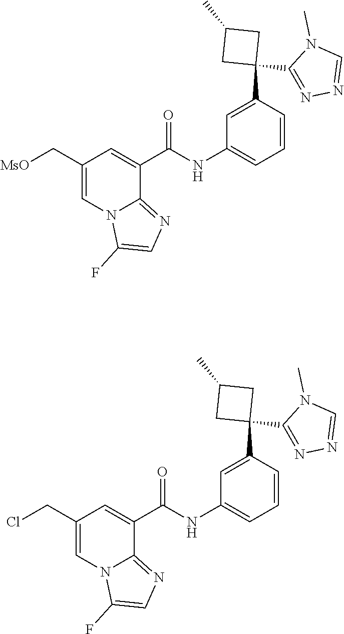 [SMILES: Cn1cnnc1[C@]1(c2cccc(NC(=O)c3cc(COS(C)(=O)=O)cn4c(F)cnc34)c2)C[C@H](C)C1 ; Cn1cnnc1[C@]1(c2cccc(NC(=O)c3cc(CCl)cn4c(F)cnc34)c2)C[C@H](C)C1]

In a mixture of the product of step 3 (333 mg, 766.46 umol), DMF (3 mL), and TEA (620.46 mg, 6.13 mmol) were added MsCl (438.99 mg, 3.83 mmol) at 0° C. The resulting mixture was stirred at 25° C. for 2 hr. The mixture was poured into 20 ml H2O, extracted with Dichloromethane (20 mL*2), washed with brine(10 mL*2), the combined organic layers were dried over Na2SO4, filtered and concentrated under reduced pressure to give 6-(chloromethyl)-3-fluoro-N-(3-((1s, 3s)-3-methyl-1-(4-methyl-4H-1,2,4-triazol-3-yl)cyclobutyl)phenyl)imidazo[1,2-a]pyridine-8-carboxamide (300 mg, crude). MS: M/e 453 (M+1)⁺.

### Step 5: 6-((((S)-1-cyclopropylethyl)amino)methyl)-3-fluoro-N-(3-((1s,3R)-3-methyl-1-(4-methyl-4H-1,2,4-triazol-3-yl)cyclobutyl)phenyl)imidazo[1,2-a]pyridine-8-carboxamide

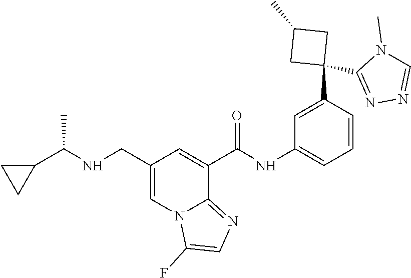 [SMILES: C[C@H](NCc1cc(C(=O)Nc2cccc([C@]3(c4nncn4C)C[C@@H](C)C3)c2)c2ncc(F)n2c1)C1CC1]

To a solution of (S)-1-cyclopropylethan-1-amine hydrochloride (107.40 mg, 883.16 umol) in DMF (1 mL) was added TEA (134.05 mg, 1.32 mmol). The mixture was stirred at 25° C. for 5 min. Then 6-(chloromethyl)-3-fluoro-N-(3-((1s, 3s)-3-methyl-1-(4-methyl-4H-1,2,4-triazol-3-yl)cyclobutyl)phenyl)imidazo[1,2-a]pyridine-8-carboxamide (100 mg, 220.79 umol) and Cs2CO3 (143.88 mg, 441.58 umol) was added at 25° C. The resulting mixture was stirred at 25° C. for 2 hr. The mixture was filtered, the filtrate was concentrated in vacuo, which was purified by prep-HPLC (column: Waters Xbridge BEH C18 100*30 mm*10 um; mobile phase: [water (NH4HCO3)-ACN]; B %: 30%-50%, 8 min) to give the product (21.25 mg, 19.19% yield). MS: M/e 502 (M+1)⁺. ¹H NMR (400 MHz, DMSO-d₆) δ=11.94 (s, 1H), 8.47 (s, 1H), 8.30 (s, 1H), 8.18 (d, J=1.2 Hz, 1H), 7.72 (s, 1H), 7.66 (br d, J=8.0 Hz, 1H), 7.61 (d, J=8.0 Hz, 1H), 7.42 (t, J=8.0 Hz, 1H), 7.16 (br d, J=8.0 Hz, 1H), 3.97-3.79 (m, 2H), 3.19 (s, 3H), 2.84 (br d, J=4.0 Hz, 2H), 2.55 (br d, J=8.0 Hz, 3H), 1.91 (br dd, J=4.0, 8.0 Hz, 1H), 1.17-1.03 (m, 6H), 0.78-0.59 (m, 1H), 0.50-0.40 (m, 1H), 0.34 (tt, J=4.0, 8.7 Hz, 1H), 0.20 (qd, J=4.0, 8.0 Hz, 1H), 0.02 (qd, J=4.0, 8.0 Hz, 1H).

### Compound A20: 3-fluoro-5-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrazolo[1,5-a]pyridine-7-carboxamide

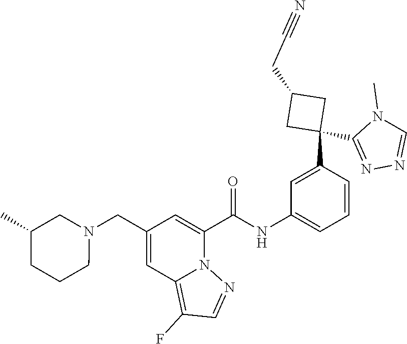 [SMILES: C[C@H]1CCCN(Cc2cc(C(=O)Nc3cccc([C@]4(c5nncn5C)C[C@@H](CC#N)C4)c3)n3ncc(F)c3c2)C1]

### Step 1: 7-bromo-5-ethenyl-3-fluoropyrazolo[1,5-a]pyridine

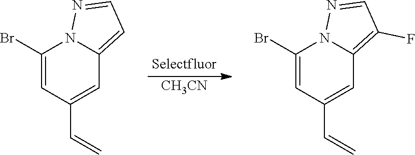

To a stirred solution of 7-bromo-5-ethenylpyrazolo[1,5-a]pyridine (700 mg, 3.078 mmol, 1 equiv, 98.1%) in MeCN were added Selectfluor (2.4 g, 6.436 mmol, 2.09 equiv, 95%) in portions at 0° C. The resulting mixture was stirred for 1.2 h at room temperature under nitrogen atmosphere. The reaction was quenched with Water at 0° C. The resulting mixture was extracted with EtOAc (3×50 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (12:1) to afford 7-bromo-5-ethenyl-3-fluoropyrazolo[1,5-a]pyridine (290 mg, 38.10%). MS: M/e 241 (M+1)⁺.

### Step 2: 5-ethenyl-3-fluoropyrazolo[1,5-a]pyridine-7-carbonitrile

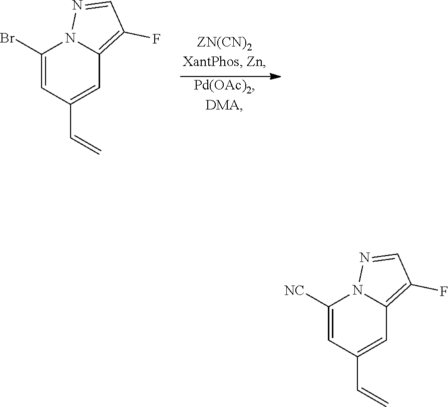

Into a 10 mL sealed tube were added 7-bromo-5-ethenyl-3-fluoropyrazolo[1,5-a]pyridine (280 mg, 1.132 mmol, 1 equiv, 97.5%), Zn(CN)₂ (224 mg, 1.812 mmol, 1.60 equiv, 95%), Zn (8 mg, 0.116 mmol, 0.10 equiv, 95%), XantPhos (276 mg, 0.453 mmol, 0.40 equiv, 95%), Pd(OAc)₂ (54 mg, 0.228 mmol, 0.20 equiv, 95%) and DMA (2 mL, 99%) at room temperature. The resulting mixture was stirred for 2 hrs at 110° C. under nitrogen atmosphere. The mixture was allowed to cool down to room temperature. The resulting mixture was concentrated under vacuum. The residue was purified by silica gel column chromatography, eluted with PE/EA (9:1) to afford 5-ethenyl-3-fluoropyrazolo[1,5-a]pyridine-7-carbonitrile (185 mg, 87.19%). MS: M/e 188 (M+1)⁺.

### Step 3: 3-fluoro-5-formylpyrazolo[1,5-a]pyridine-7-carbonitrile

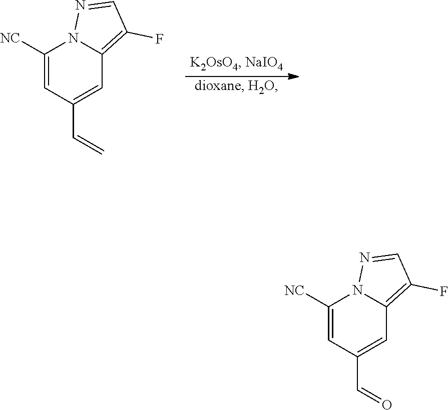

To a stirred solution of 5-ethenyl-3-fluoropyrazolo[1,5-a]pyridine-7-carbonitrile (180 mg, 0.961 mmol, 1 equiv, 99.9%) in dioxane (3 mL, 99%) and H₂O (3 mL, 99%) was added K₂OsO₄·2H₂O (38 mg, 0.098 mmol, 0.10 equiv, 95%) and NaIO₄ (433 mg, 1.923 mmol, 2.00 equiv, 95%). The resulting mixture was stirred for 4 hrs at room temperature. The reaction was quenched with Water. The resulting mixture was extracted with EtOAc (3×15 mL). The combined organic layers were washed with brine (1×15 mL), dried over anhydrous Na₂SO₄. After filtration, the filtrate was concentrated under reduced pressure. This resulted in 3-fluoro-5-formylpyrazolo[1,5-a]pyridine-7-carbonitrile (170 mg, 90.09%). MS: M/e 190 (M+1)⁺.

### Step 4: 3-fluoro-5-{[(3S)-3-methylpiperidin-1-yl]methyl}pyrazolo[1,5-a]pyridine-7-carbonitrile

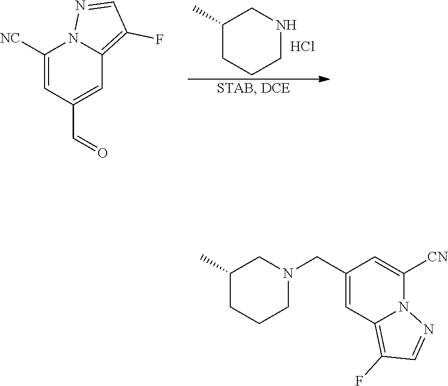

Into a 20 mL sealed tube were added 3-fluoro-5-formylpyrazolo[1,5-a]pyridine-7-carbonitrile (160 mg, 0.815 mmol, 1 equiv, 96.3%) and (3S)-3-methylpiperidine hydrochloride (233 mg, 1.632 mmol, 2.00 equiv, 95%) and DCE (5 mL, 99%) at room temperature. The resulting mixture was stirred for 4 hrs at 70° C. under nitrogen atmosphere. The mixture was allowed to cool down to room temperature. To the above mixture was added STAB (275 mg, 1.233 mmol, 1.51 equiv, 95%) in portions at room temperature. The resulting mixture was stirred for additional 3 hrs at 40° C. The mixture was allowed to cool down to room temperature. The resulting mixture was concentrated under vacuum. The residue was purified by silica gel column chromatography, eluted with CH₂Cl₂/MeOH (97:3) to afford 3-fluoro-5-{[(3S)-3-methylpiperidin-1-yl]methyl}pyrazolo[1,5-a]pyridine-7-carbonitrile (145 mg, 63.79%). MS: M/e 273 (M+1)⁺.

### Step 5: 3-fluoro-5-{[(3S)-3-methylpiperidin-1-yl]methyl}pyrazolo[1,5-a]pyridine-7-carboxylic acid

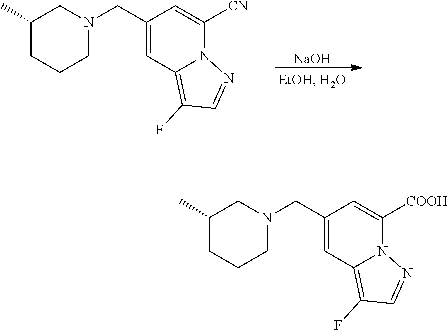 [SMILES: C[C@H]1CCCN(Cc2cc(C#N)n3ncc(F)c3c2)C1 ; "NaOH" ; C[C@H]1CCCN(Cc2cc(C(=O)O)n3ncc(F)c3c2)C1]

To a stirred solution of 3-fluoro-5-{[(3S)-3-methylpiperidin-1-yl]methyl}pyrazolo[1,5-a]pyridine-7-carbonitrile (130 mg, 0.466 mmol, 1 equiv, 97.6%) in EtOH and H₂O was added NaOH (60 mg, 1.425 mmol, 3.06 equiv, 95%) in portions. The resulting mixture was stirred for 3 hrs at 80° C. The mixture was allowed to cool down to room temperature. The resulting mixture was concentrated under vacuum. The mixture was acidified to pH 3 with HCl (1 M). The residue was purified by reverse flash chromatography with the following conditions: column, C18 silica gel; mobile phase, MeCN in Water (0.1% NH₃·H₂O), 10% to 40% gradient in 20 min; detector, UV 220 nm. This resulted in 3-fluoro-5-{[(3S)-3-methylpiperidin-1-yl]methyl}pyrazolo[1,5-a]pyridine-7-carboxylic acid (115 mg, 83.88%). MS: M/e 292 (M+1)⁺.

### Step 6: 3-fluoro-5-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrazolo[1,5-a]pyridine-7-carboxamide

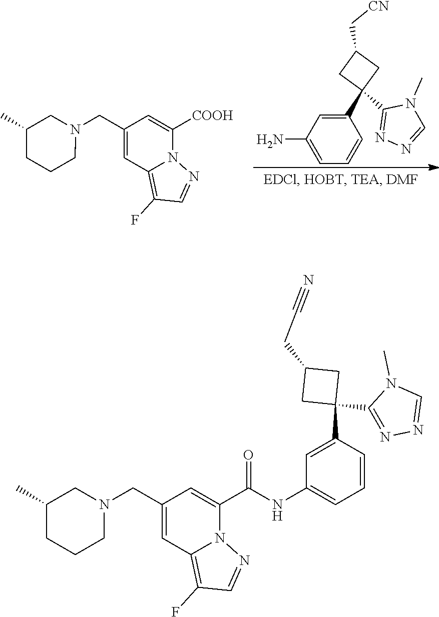 [SMILES: C[C@H]1CCCN(Cc2cc(C(=O)O)n3ncc(F)c3c2)C1 ; Cn1cnnc1[C@]1(c2cccc(N)c2)C[C@H](CC#N)C1 ; C[C@H]1CCCN(Cc2cc(C(=O)Nc3cccc([C@]4(c5nncn5C)C[C@@H](CC#N)C4)c3)n3ncc(F)c3c2)C1]

To a stirred mixture of 3-fluoro-5-{[(3S)-3-methylpiperidin-1-yl]methyl}pyrazolo[1,5-a]pyridine-7-carboxylic acid (35 mg, 0.119 mmol, 1 equiv, 99.0%) and HOBT (26 mg, 0.183 mmol, 1.54 equiv, 95%), EDCI (36.00 mg, 0.178 mmol, 1.5 equiv, 95%), Et₃N (38 mg, 0.357 mmol, 3.00 equiv, 95%) in DMF was added 2-[(1s, 3s)-3-(3-aminophenyl)-3-(4-methyl-1,2,4-triazol-3-yl)cyclobutyl]acetonitrile (22 mg, 0.081 mmol, 0.68 equiv, 98.3%). The resulting mixture was stirred for 8 hrs at 40° C. The residue was purified by reverse flash chromatography with the following conditions: column, C18 silica gel; mobile phase, MeCN in Water (0.1% NH₃·H₂O), 10% to 95% gradient in 15 min; detector, UV 254 nm. The crude product (45 mg) was purified by Prep-HPLC with the following conditions (2 #SHIMADZU): Column, XBridge Prep OBD C18 Column, 30*150 mm, 5 μm; mobile phase, Water (10 mmol/L NH4HCO₃+0.1% NH₃·H₂O) and ACN (45% ACN up to 75% in 8 min); This resulted in 3-fluoro-5-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrazolo[1,5-a]pyridine-7-carboxamide (20.6 mg, 30.63%). MS: M/e 541 (M+1)⁺. ¹H NMR (300 MHz, DMSO-d₆) δ 11.93 (s, 1H), 8.37-8.29 (m, 2H), 7.81 (s, 1H), 7.74-7.62 (m, 3H), 7.48-7.41 (m, 1H), 7.21 (d, J=7.8 Hz, 1H), 3.55 (s, 2H), 3.22 (s, 3H), 2.95-2.65 (m, 9H), 2.02-1.87 (m, 1H), 1.75-1.39 (m, 5H), 0.98-0.70 (m, 4H).

### Compound A21: 3-chloro-N-(3-((1s,3R)-3-(cyanomethyl)-1-(4-methyl-4H-1,2,4-triazol-3-yl)cyclobutyl)phenyl)-5-(((S)-3-methylpiperidin-1-yl)methyl)pyrazolo[1,5-a]pyridine-7-carboxamide

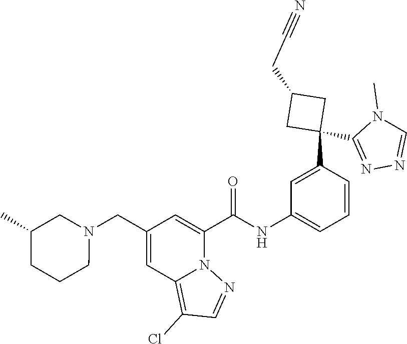 [SMILES: C[C@H]1CCCN(Cc2cc(C(=O)Nc3cccc([C@]4(c5nncn5C)C[C@@H](CC#N)C4)c3)n3ncc(Cl)c3c2)C1]

### Step 1: 7-bromo-3-chloro-5-vinylpyrazolo[1,5-a]pyridine

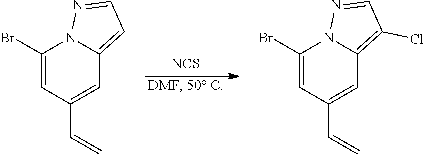

A solution of 7-bromo-5-ethenylpyrazolo[1,5-a]pyridine (900 mg, 3.833 mmol, 1 equiv, 95%) and NCS (1077.47 mg, 7.666 mmol, 2 equiv, 95%) in DMF was stirred for 2 hrs at 50° C. under nitrogen atmosphere. The resulting mixture was concentrated under reduced pressure. The residue was purified by silica gel column chromatography, eluted with PE/EA (5:1) to afford 7-bromo-3-chloro-5-ethenylpyrazolo[1,5-a]pyridine (820 mg, 78.92%). MS: M/e 257 (M+1)⁺.

### Step 2: 3-chloro-5-vinylpyrazolo[1,5-a]pyridine-7-carbonitrile

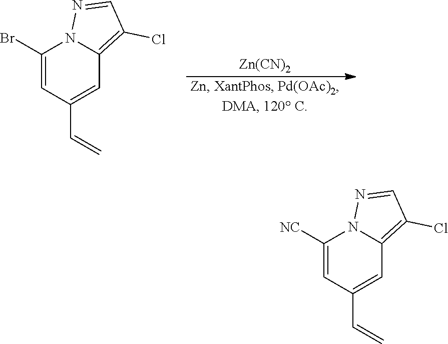

To a stirred solution of 7-bromo-3-chloro-5-ethenylpyrazolo[1,5-a]pyridine (800 mg, 2.951 mmol, 1 equiv, 95%) and Zn(CN)₂ (583.63 mg, 4.722 mmol, 1.6 equiv, 95%) in 1,4-dioxane were added Zn (223.42 mg, 3.246 mmol, 1.1 equiv, 95%), XantPhos (719.02 mg, 1.180 mmol, 0.4 equiv, 95%) and Pd(OAc)₂ (139.49 mg, 0.590 mmol, 0.2 equiv, 95%) at room temperature under nitrogen atmosphere. The resulting mixture was stirred for additional 1 h at 110° C. The resulting mixture was concentrated under reduced pressure. The residue was purified by silica gel column chromatography, eluted with PE/EA (5:1) to afford 3-chloro-5-ethenylpyrazolo[1,5-a]pyridine-7-carbonitrile (490 mg, 77.46%). MS: M/e 204 (M+1)⁺.

### Step 3: 3-chloro-5-formylpyrazolo[1,5-a]pyridine-7-carbonitrile

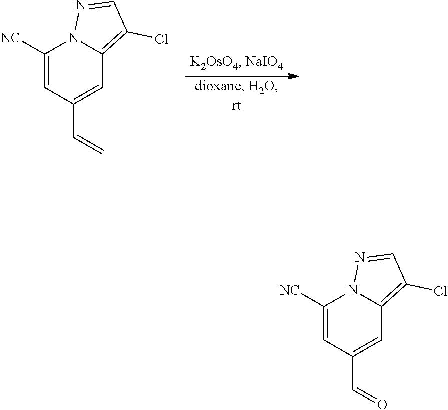

To a stirred solution of 3-chloro-5-ethenylpyrazolo[1,5-a]pyridine-7-carbonitrile (440 mg, 2.053 mmol, 1 equiv, 95%) and K₂OsO₄.2H₂O (79.61 mg, 0.205 mmol, 0.1 equiv, 95%) in 1,4-dioxane/H₂O was added NaIO₄ (924.34 mg, 4.106 mmol, 2 equiv, 95%) at room temperature under nitrogen atmosphere. The resulting mixture was stirred for additional 2 hrs at room temperature. The aqueous layer was extracted with EtOAc (2×50 mL). The resulting mixture was concentrated under reduced pressure. The residue was purified by silica gel column chromatography, eluted with PE/EA (3:1) to afford 3-chloro-5-formylpyrazolo[1,5-a]pyridine-7-carbonitrile (330 mg, 74.28%). MS: M/e 206 (M+1)⁺.

### Step 4: (S)-3-chloro-5-((3-methylpiperidin-1-yl)methyl)pyrazolo[1,5-a]pyridine-7-carbonitrile

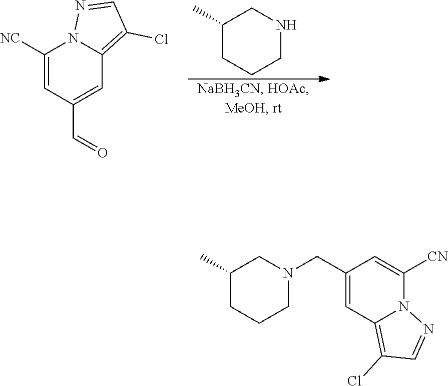

A solution of 3-chloro-5-formylpyrazolo[1,5-a]pyridine-7-carbonitrile (310 mg, 1.432 mmol, 1 equiv, 95%) and (3S)-3-methylpiperidine (299.07 mg, 2.864 mmol, 2 equiv, 95%) in DCE was stirred for 2 hrs at 70° C. under nitrogen atmosphere. To the above mixture was added STAB (479.34 mg, 2.148 mmol, 1.5 equiv, 95%) at room temperature. The resulting mixture was stirred for additional 3 hrs at 40° C. The resulting mixture was concentrated under reduced pressure. The residue was purified by silica gel column chromatography, eluted with PE/EA (1:1) to afford 3-chloro-5-{[(3S)-3-methylpiperidin-1-yl]methyl}pyrazolo[1,5-a]pyridine-7-carbonitrile (280 mg, 64.31%). MS: M/e 289 (M+1)⁺.

### Step 5: (S)-3-chloro-5-((3-methylpiperidin-1-yl)methyl)pyrazolo[1,5-a]pyridine-7-carboxylic acid

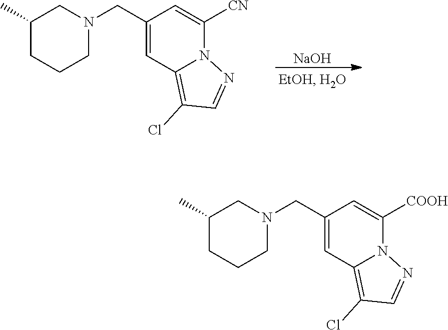 [SMILES: C[C@H]1CCCN(Cc2cc(C#N)n3ncc(Cl)c3c2)C1 ; "NaOH" ; C[C@H]1CCCN(Cc2cc(C(=O)O)n3ncc(Cl)c3c2)C1]

A solution of 3-chloro-5-{[(3S)-3-methylpiperidin-1-yl]methyl}pyrazolo[1,5-a]pyridine-7-carbonitrile (260 mg, 0.855 mmol, 1 equiv, 95%) and NaOH (108.03 mg, 2.565 mmol, 3 equiv, 95%) in EtOH/H₂O was stirred for 2 hrs at 80° C. under nitrogen atmosphere. The resulting mixture was concentrated under reduced pressure. The residue was purified by reverse flash chromatography with the following conditions: column, C18 silica gel; mobile phase, MeCN in Water (0.1% TFA), 0% to 100% gradient in 30 min; detector, UV 254 nm. This resulted in 3-chloro-5-{[(3S)-3-methylpiperidin-1-yl]methyl}pyrazolo[1,5-a]pyridine-7-carboxylic acid (170 mg, 58.12%). MS: M/e 308 (M+1)⁺.

### Step 6: 3-chloro-N-(3-((1s,3R)-3-(cyanomethyl)-1-(4-methyl-4H-1,2,4-triazol-3-yl)cyclobutyl)phenyl)-5-(((S)-3-methylpiperidin-1-yl)methyl)pyrazolo[1,5-a]pyridine-7-carboxamide

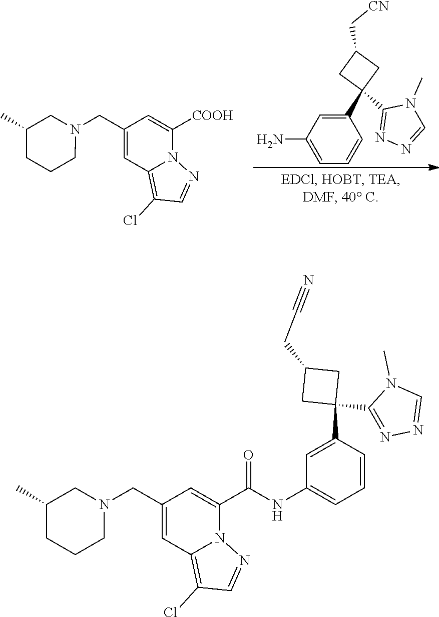 [SMILES: C[C@H]1CCCN(Cc2cc(C(=O)O)n3ncc(Cl)c3c2)C1 ; Cn1cnnc1[C@]1(c2cccc(N)c2)C[C@H](CC#N)C1 ; C[C@H]1CCCN(Cc2cc(C(=O)Nc3cccc([C@]4(c5nncn5C)C[C@@H](CC#N)C4)c3)n3ncc(Cl)c3c2)C1]

To a stirred solution of 3-chloro-5-{[(3S)-3-methylpiperidin-1-yl]methyl}pyrazolo[1,5-a]pyridine-7-carboxylic acid (34.54 mg, 0.106 mmol, 1.5 equiv, 95%) and Et₃N (22.71 mg, 0.213 mmol, 3 equiv, 95%) in DMF were added EDCI (21.51 mg, 0.106 mmol, 1.5 equiv, 95%) and HOBT (15.16 mg, 0.106 mmol, 1.5 equiv, 95%) at room temperature under nitrogen atmosphere. The resulting mixture was stirred for additional 20 min at room temperature. To the above mixture was added 2-[(1s, 3s)-3-(3-aminophenyl)-3-(4-methyl-1,2,4-triazol-3-yl)cyclobutyl]acetonitrile (20 mg, 0.071 mmol, 1.00 equiv, 95%) at room temperature. The resulting mixture was stirred for additional 2 hrs at 40° C. The resulting mixture was concentrated under reduced pressure. The crude product was purified by Chiral-Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (55% ACN up to 70% in 8 min). This resulted in 3-chloro-5-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrazolo[1,5-a]pyridine-7-carboxamide (7 mg, 17.22%). MS: M/e 557 (M+1)⁺. ¹H NMR (300 MHz, DMSO-d₆) δ 11.82 (s, 1H), 8.34 (m, 2H), 7.76-7.64 (m, 4H), 7.43 (m, 1H), 7.26-7.16 (m, 1H), 3.57 (s, 2H), 3.20 (s, 3H), 2.90-2.66 (m, 9H), 1.94 (m, 1H), 1.70-1.39 (m, 5H), 0.81 (m, 4H).

### Compound A22: 6-(((cyclobutylmethyl)amino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

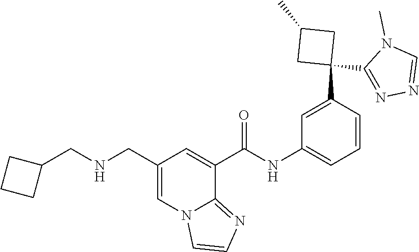 [SMILES: Cn1cnnc1[C@]1(c2cccc(NC(=O)c3cc(CNCC4CCC4)cn4ccnc34)c2)C[C@H](C)C1]

### Step 1: 6-formyl-N-(3-((1s, 3s)-3-methyl-1-(4-methyl-4H-1,2,4-triazol-3-yl)cyclobutyl)phenyl)imidazo[1,2-a]pyridine-8-carboxamide

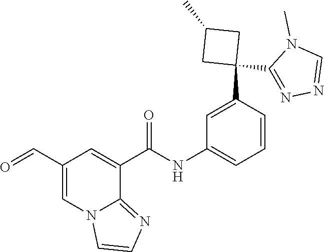 [SMILES: Cn1cnnc1[C@]1(c2cccc(NC(=O)c3cc(C=O)cn4ccnc34)c2)C[C@H](C)C1]

To a solution of 6-formylimidazo[1,2-a]pyridine-8-carboxylic acid (250 mg, 1.31 mmol) in DMF (1 mL) was added HATU (599.87 mg, 1.58 mmol), DIEA (339.84 mg, 2.63 mmol). After addition, the mixture was stirred at this temperature for 5 min, and then 3-((1s, 3s)-3-methyl-1-(4-methyl-4H-1,2,4-triazol-3-yl)cyclobutyl)aniline (318.58 mg, 1.31 mmol) was added dropwise at 25° C. The resulting mixture was stirred at 25° C. for 2 hrs. The reaction mixture was quenched by addition H₂O 5 mL at 20° C., and then extracted with DCM 30 mL (10 mL*3). The combined organic layers were washed with brine 30 mL (10 mL*3), dried over Na₂SO₄, filtered and concentrated under reduced pressure to give a residue, which was purified by Prep-HPLC (column: Waters Xbridge BEH C18 100*30 mm*10 um; mobile phase: [water (NH₄HCO₃)-ACN]; B %: 25%-55%, 8 min) to give the product (350 mg, 64.23% yield). MS: M/e 415 (M+1)⁺. ¹H NMR (400 MHz, DMSO-d₆) δ=12.15 (s, 1H), 10.05 (s, 1H), 9.56 (d, J=1.6 Hz, 1H), 8.41 (d, J=1.6 Hz, 1H), 8.37 (d, J=1.6 Hz, 1H), 8.30 (s, 1H), 7.96 (d, J=1.6 Hz, 1H), 7.75-7.70 (m, 1H), 7.68 (s, 1H), 7.43 (t, J=8.0 Hz, 1H), 7.18 (d, J=8.0 Hz, 1H), 3.20 (s, 3H), 2.84 (br d, J=4.0 Hz, 2H), 2.55 (br d, J=8.0 Hz, 3H), 1.09 (br d, J=8.0 Hz, 3H).

### Step 2: 6-(((cyclobutylmethyl)amino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

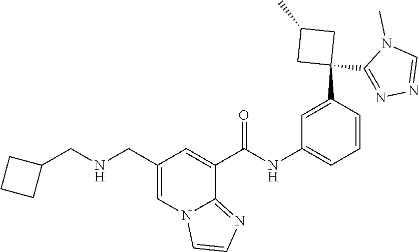 [SMILES: Cn1cnnc1[C@]1(c2cccc(NC(=O)c3cc(CNCC4CCC4)cn4ccnc34)c2)C[C@H](C)C1]

To a solution cyclobutylmethanamine hydrochloride (88.02 mg, 723.84 umol,) in DCE (2 mL) was added TEA (73.24 mg, 723.84 umol). After addition, the mixture was stirred at this temperature for 5 min, and then 6-formyl-N-(3-((1s, 3s)-3-methyl-1-(4-methyl-4H-1,2,4-triazol-3-yl)cyclobutyl)phenyl)imidazo[1,2-a]pyridine-8-carboxamide (150 mg, 361.92 umol) and NaBH(OAc)₃ (191.76 mg, 904.79 umol) was added dropwise at 25° C. The mixture was stirred at 25° C. for 12 hrs. The reaction mixture was filtered and the filter was concentrated, which was purified by Prep-HPLC (column: Waters Xbridge Prep OBD C18 150*40 mm*10 um; mobile phase: [water (NH₄HCO₃)-ACN]; B %: 35%-65%, 8 min) to give the product (85.53 mg, 48.87% yield). MS: M/e 484 (M+1)⁺. ¹H NMR (400 MHz, DMSO-d₆) δ=12.41 (s, 1H), 8.71 (s, 1H), 8.30 (s, 1H), 8.14 (dd, J=1.2, 4.0 Hz, 2H), 7.78 (d, J=1.2 Hz, 1H), 7.73 (s, 1H), 7.70-7.62 (m, 1H), 7.42 (t, J=8.0 Hz, 1H), 7.15 (br d, J=8.0 Hz, 1H), 3.76 (s, 2H), 3.25-3.15 (m, 4H), 2.84 (br d, J=4.0 Hz, 2H), 2.61-2.52 (m, 5H), 2.41 (td, J=8.0, 16.0 Hz, 1H), 2.05-1.91 (m, 2H), 1.86-1.72 (m, 2H), 1.71-1.58 (m, 2H), 1.09 (br d, J=8.0 Hz, 3H).

### Compound A23: N-(3-((1s,3R)-3-(cyanomethyl)-1-(4-methyl-4H-1,2,4-triazol-3-yl)cyclobutyl)phenyl)-6-((((S)-3,3-difluorocyclopentyl)amino)methyl)imidazo[1,2-a]pyridine-8-carboxamide

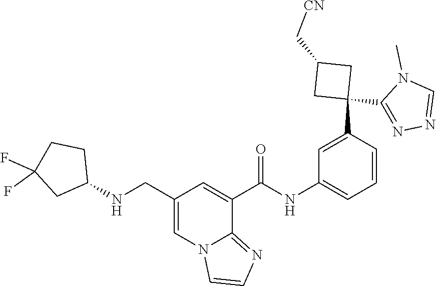 [SMILES: Cn1cnnc1[C@]1(c2cccc(NC(=O)c3cc(CN[C@H]4CCC(F)(F)C4)cn4ccnc34)c2)C[C@H](CC#N)C1]

### Step 1: N-(3-((1s, 3s)-3-(cyanomethyl)-1-(4-methyl-4H-1,2,4-triazol-3-yl)cyclobutyl)phenyl)-6-formylimidazo[1,2-a]pyridine-8-carboxamide

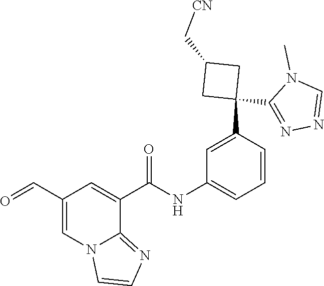 [SMILES: Cn1cnnc1[C@]1(c2cccc(NC(=O)c3cc(C=O)cn4ccnc34)c2)C[C@H](CC#N)C1]

To a solution of 6-formylimidazo[1,2-a]pyridine-8-carboxylic acid (88.91 mg, 467.59 mol) in DMF (2 mL) was added HATU (213.35 mg, 561.11 mol), DIEA (120.86 mg, 935.18 mol) and 2-((1s, 3s)-3-(3-aminophenyl)-3-(4-methyl-4H-1,2,4-triazol-3-yl)cyclobutyl)acetonitrile (100 mg, 374.07 mol). The mixture was stirred at 25° C. for 2 hrs. The reaction mixture was quenched by addition H₂O (5 mL) at 20° C., and then extracted with DCM 30 mL (10 mL*3). The combined organic layers were washed with brine 30 mL (10 mL*3), dried over Na₂SO₄, filtered and concentrated under reduced pressure to give a residue, which was purified by Prep-HPLC (column: Waters Xbridge Prep OBD C18 150*40 mm*10 um; mobile phase: [water (NH4HCO₃)-ACN]; B %: 20%-50%, 8 min) to give the product (50 mg, 24.33% yield). MS: M/e 440 (M+1)⁺, ¹H NMR (400 MHz, DMSO-d₆) δ=12.16 (s, 1H), 10.06 (s, 1H), 9.57 (d, J=1.6 Hz, 1H), 8.42 (d, J=1.6 Hz, 1H), 8.38 (d, J=1.2 Hz, 1H), 8.32 (s, 1H), 7.97 (d, J=1.2 Hz, 1H), 7.76-7.68 (m, 2H), 7.46 (t, J=8.0 Hz, 1H), 7.23 (d, J=8.0 Hz, 1H), 3.22 (s, 3H), 2.90 (br d, J=4.0 Hz, 2H), 2.76 (br d, J=8.0 Hz, 3H), 2.71 (br s, 2H).

### Step 2: N-(3-((1s,3R)-3-(cyanomethyl)-1-(4-methyl-4H-1,2,4-triazol-3-yl)cyclobutyl)phenyl)-6-((((S)-3,3-difluorocyclopentyl)amino)methyl)imidazo[1,2-a]pyridine-8-carboxamide

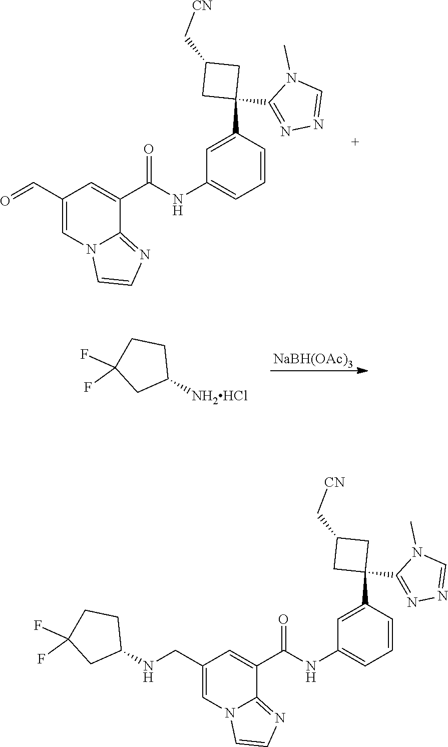

A solution of N-(3-((1s, 3s)-3-(cyanomethyl)-1-(4-methyl-4H-1,2,4-triazol-3-yl)cyclobutyl)phenyl)-6-formylimidazo[1,2-a]pyridine-8-carboxamide (60 mg, 0.14 mmol), (S)-3,3-difluorocyclopentan-1-amine hydrochloride (43 mg, 0.28 mmol) and TEA (28 mg, 0.28 mmol) in DCM (3 mL) was stirred at r.t for 30 mins. Then it was cooled under ice bath, and NaBH(OAc)₃ (57 mg, 0.28 mmol) was added. The reaction mixture was stirred at r.t for 3 hrs, then more of NaBH(OAc)₃ (57 mg, 0.28 mmol) was added and it continued to stir overnight. The reaction mixture was added with water (3 mL), basified with NaHCO₃ solution to pH=7-8 and extracted with DCM (6 mL). The organic layer was dried, concentrated and purified by prep.TLC (DCM:MeOH=8:1) to get the product (25 mg, 34%). ¹H NMR (400 MHz, CD₃OD) δ 8.65 (s, 1H), 8.33 (s, 1H), 8.23 (s, 1H), 7.96 (d, J=8.0 Hz, 2H), 7.72 (s, 1H), 7.71 (d, J=4.0 Hz, 1H), 7.46 (t, J=8.0 Hz, 1H), 7.19 (d, J=8.0 Hz, 1H), 3.90 (s, 2H), 3.40-3.35 (m, 1H), 3.34 (s, 3H), 3.05-3.02 (m, 2H), 2.96-2.86 (m, 3H), 2.67 (d, J=8.0 Hz, 2H), 2.53-2.42 (m, 1H), 2.26-2.16 (m, 2H), 2.06-1.92 (m, 2H), 1.75-1.68 (m, 1H). ppm. MS: M/e 545 (M+1)+

### Compound A24: N-(3-((1r, 3r)-3-cyano-1-((4-methyl-4H-1,2,4-triazol-3-yl)methyl)cyclobutyl)phenyl)-6-((((1-methylcyclopentyl)methyl)amino)methyl)imidazo[1,2-a]pyridine-8-carboxamide

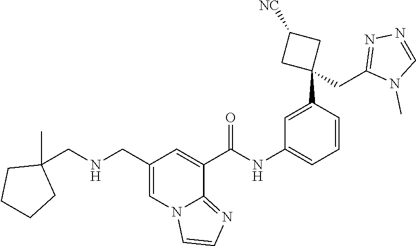 [SMILES: Cn1cnnc1C[C@]1(c2cccc(NC(=O)c3cc(CNCC4(C)CCCC4)cn4ccnc34)c2)C[C@H](C#N)C1]

### Step A: 6-formylimidazo[1,2-a]pyridine-8-carboxylic acid

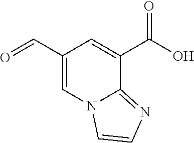 [SMILES: O=Cc1cc(C(=O)O)c2nccn2c1]

To a solution of methyl 6-formylimidazo[1,2-a]pyridine-8-carboxylate (300 mg, 1.47 mmol) in MeOH (10 mL)/H₂O (1 mL) was added LiOH H₂O (312 mg, 7.43 mmol). The reaction mixture was stirred for 12 h at 25° C. After completed, the mixture was filtered and the solid was collected and washed with MeOH (10 mL), dried to afford the title compound (240 mg, 86%). MS: M/e 191 (M+H)⁺.

### Step B: N-(3-((1r, 3r)-3-cyano-1-((4-methyl-4H-1,2,4-triazol-3-yl)methyl)cyclobutyl)phenyl)-6-formylimidazo[1,2-a]pyridine-8-carboxamide

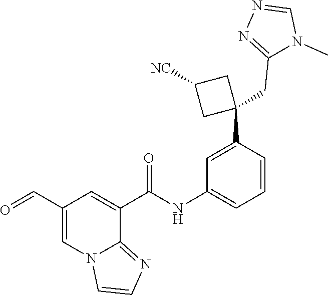 [SMILES: Cn1cnnc1C[C@]1(c2cccc(NC(=O)c3cc(C=O)cn4ccnc34)c2)C[C@H](C#N)C1]

To a solution of 6-formylimidazo[1,2-a]pyridine-8-carboxylic acid (240 mg, 1.26 mmol), (1r, 3r)-3-(3-aminophenyl)-3-((4-methyl-4H-1,2,4-triazol-3-yl)methyl)cyclobutane-1-carbonitrile (330 mg, 1.23 mmol), DIEA (320 mg, 2.48 mmol) in DMF (5 mL) was added HATU (720 mg, 1.89 mmol). The reaction mixture was stirred for 2 h at 25° C. After completed, the reaction was quenched with aq NH4C1 (20 mL) and extracted with DCM (3×30 mL), dried over Na₂SO₄ and concentrated under vacuum. The residue was purified by column chromatography on silica gel eluting with methanol in dichloromethane (5%) to afford the title compound (300 mg, 54%). MS: M/e 440. (M+H)⁺.

### Step C: N-(3-((1r, 3r)-3-cyano-1-((4-methyl-4H-1,2,4-triazol-3-yl)methyl)cyclobutyl)phenyl)-6-((((1-methylcyclopentyl)methyl)amino)methyl)imidazo[1,2-a]pyridine-8-carboxamide

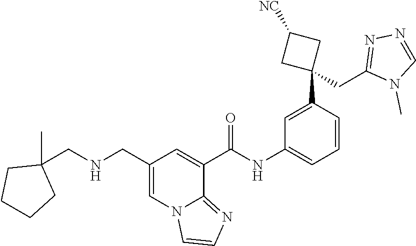 [SMILES: Cn1cnnc1C[C@]1(c2cccc(NC(=O)c3cc(CNCC4(C)CCCC4)cn4ccnc34)c2)C[C@H](C#N)C1]

To a solution of (1-methylcyclopentyl)methanamine hydrochloride (51.08 mg, 341.31 mol) in DCE (2 mL) was added TEA (34.54 mg, 341.31 mol). After addition, the mixture was stirred at this temperature for 5 min, and then N-(3-((1r, 3r)-3-cyano-1-((4-methyl-4H-1,2,4-triazol-3-yl)methyl)cyclobutyl)phenyl)-6-formylimidazo[1,2-a]pyridine-8-carboxamide (100 mg, 227.54 mol) and NaBH(OAc)₃ (144.68 mg, 682.63 mol) was added dropwise at 25° C. The mixture was stirred at 25° C. for 16 hrs. The reaction mixture was filtered and the filter was concentrated, which was purified by Prep-HPLC (column=Waters Xbridge Prep OBD C18 (150*40 mm*10 um); mobile phase=water (NH4HCO₃)-ACN, B %=40%-70%; 8 min) to give the product (32.13 mg, 26.31% yield). MS: M/e 537 (M+1)⁺, ¹H NMR (400 MHz, DMSO-d₆) δ 12.41 (s, 1H), 8.72 (d, J=1.2 Hz, 1H), 8.21-8.16 (m, 2H), 8.15 (d, J=1.2 Hz, 1H), 7.78 (d, J=1.2 Hz, 1H), 7.69 (dd, J=1.2, 8.1 Hz, 1H), 7.41 (t, J=2.0 Hz, 1H), 7.31 (t, J=8.0 Hz, 1H), 6.73 (d, J=8.0 Hz, 1H), 3.80 (s, 2H), 3.31 (s, 2H), 3.29-3.21 (m, 1H), 2.93-2.83 (m, 4H), 2.80 (s, 3H), 2.36 (s, 2H), 2.30-2.14 (m, 1H), 1.63-1.41 (m, 6H), 1.31-1.18 (m, 2H), 0.97 (s, 3H) ppm.

### Compound A25: N-(3-((1s, 3s)-3-(cyanomethyl)-1-(4-methyl-4H-1,2,4-triazol-3-yl)cyclobutyl)phenyl)-6-(((1-methylcyclopropyl)amino)methyl)imidazo[1,2-a]pyridine-8-carboxamide

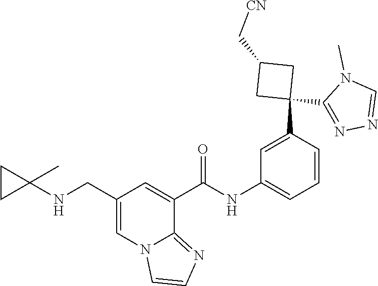 [SMILES: Cn1cnnc1[C@]1(c2cccc(NC(=O)c3cc(CNC4(C)CC4)cn4ccnc34)c2)C[C@H](CC#N)C1]

To a solution of N-(3-((1s, 3s)-3-(cyanomethyl)-1-(4-methyl-4H-1,2,4-triazol-3-yl)cyclobutyl)phenyl)-6-formylimidazo[1,2-a]pyridine-8-carboxamide (30 mg, 68.26 umol), 1-methylcyclopropan-1-amine hydrochloride (10.36 mg, 102.39 umol) and trimethylamine (7.28 mg, 102.39 umol) in DCM (1 mL). NaBH(OAc)₃ (43.40 mg, 204.79 umol) was added. The mixture was stirred for 16 hrs at RT under N₂. The mixture was poured into water (1 mL) and extracted with DCM (2 mL, 1 mL). The combined organic phase was washed with brine (1 mL), dried over Na₂SO₄, filtered and concentrated to give the residue, which was purified by Prep-HPLC (water (NH4HCO₃)-CAN, Waters Xbridge BEH C18 100*30 mm*10 um, B %=50%-80%; 8 min) to give the product (14.55 mg). MS: M/e 495 (M+1)+; ¹H NMR (400 MHz, DMSO-d₆) δ=12.42 (s, 1H), 8.72 (s, 1H), 8.33 (s, 1H), 8.13 (m, 2H), 7.78 (d, J=1.6 Hz, 1H), 7.76-7.75 (m, 1H), 7.69 (d, J=8 Hz, 1H), 7.45 (t, J=8 Hz, 1H), 7.20 (d, J=8 Hz, 1H), 3.83 (s, 2H), 3.23 (s, 3H), 2.96-2.82 (m, 2H), 2.80-2.69 (m, 5H), 1.28 (s, 3H), 0.55-0.49 (m, 2H), 0.36-0.31 (m, 2H) ppm.

### Compound A26: N-(3-((1s, 3s)-3-(cyanomethyl)-1-(4-methyl-4H-1,2,4-triazol-3-yl)cyclobutyl)phenyl)-6-(((cyclopentylmethyl)amino)methyl)imidazo[1,2-a]pyridine-8-carboxamide

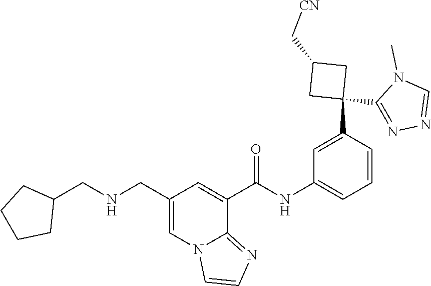 [SMILES: Cn1cnnc1[C@]1(c2cccc(NC(=O)c3cc(CNCC4CCCC4)cn4ccnc34)c2)C[C@H](CC#N)C1]

The compound A26 was synthesized starting from the corresponding starting materials according the similar procedures described as those of Compound A23 to give the product (14.68 mg). MS: M/e 523 (M+1); ¹H NMR (400 MHz, DMSO-d₆) δ=12.42 (s, 1H), 8.73 (s, 1H), 8.33 (s, 1H), 8.16 (d, J=8.0 Hz, 2H), 7.79 (d, J=4 Hz, 1H), 7.75 (s, 1H), 7.70 (d, J=8.0 Hz, 1H), 7.45 (t, J=8 Hz, 1H), 7.21 (d, J=8.0 Hz, 1H), 3.79 (s, 2H), 3.40-3.35 (m, 1H), 3.30-3.25 (m, 1H), 3.23 (s, 3H), 2.96-2.82 (m, 2H), 2.80-2.69 (m, 5H), 2.45-2.43 (m, 1H), 2.05-1.94 (m, 1H), 1.76-1.65 (m, 2H), 1.59-1.43 (m, 4H), 1.23-1.12 (m, 2H) ppm.

### Compound A27: N-(3-((1s,3R)-3-(cyanomethyl)-1-(4-methyl-4H-1,2,4-triazol-3-yl)cyclobutyl)phenyl)-6-((((S)-1-cyclopropylethyl)amino)methyl)imidazo[1,2-a]pyridine-8-carboxamide

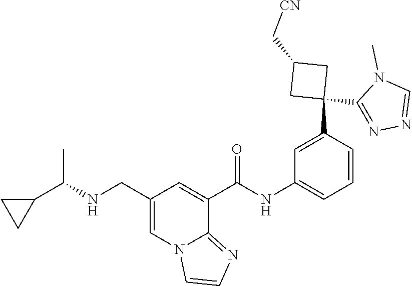 [SMILES: C[C@H](NCc1cc(C(=O)Nc2cccc([C@]3(c4nncn4C)C[C@@H](CC#N)C3)c2)c2nccn2c1)C1CC1]

The compound A27 was synthesized starting from the corresponding starting materials according the similar procedures described as those of Compound A23 to give the product (2.79 mg, 12.05% yield). MS: M/e 509 (M+1)⁺; ¹H NMR (400 MHz, DMSO-d₆) δ 12.42 (s, 1H), 8.74 (s, 1H), 8.32 (s, 1H), 8.17 (s, 1H), 8.16-8.13 (m, 1H), 7.79-7.78 (m, 1H), 7.75 (s, 1H), 7.72-7.67 (m, 1H), 7.44 (t, J=8.0 Hz, 1H), 7.20 (d, J=8.0 Hz, 1H), 3.92-3.81 (m, 2H), 3.22 (s, 3H), 2.93-2.85 (m, 2H), 2.82-2.67 (m, 5H), 2.40-2.25 (m, 1H), 1.96-1.86 (m, 1H), 1.13-1.08 (m, 3H), 0.75-0.64 (m, 1H), 0.53-0.39 (m, 1H), 0.39-0.30 (m, 1H), 0.25-0.19 (m, 1H), 0.08-−0.03 (m, 1H) ppm.

### Compound A28: 3-chloro-N-(3-((1s, 3s)-3-(cyanomethyl)-1-(4-methyl-4H-1,2,4-triazol-3-yl)cyclobutyl)phenyl)-6-((((1-methylcyclopentyl)methyl)amino)methyl)imidazo[1,2-a]pyridine-8-carboxamide

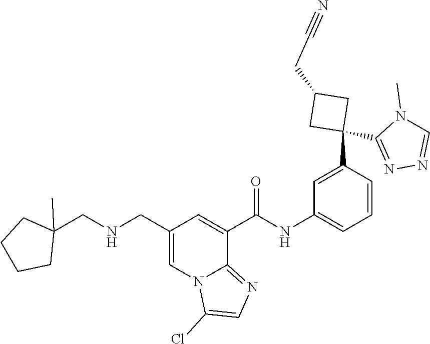 [SMILES: Cn1cnnc1[C@]1(c2cccc(NC(=O)c3cc(CNCC4(C)CCCC4)cn4c(Cl)cnc34)c2)C[C@H](CC#N)C1]

### Step A: methyl 3-chloro-6-formylimidazo[1,2-a]pyridine-8-carboxylate

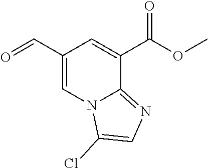 [SMILES: COC(=O)c1cc(C=O)cn2c(Cl)cnc12]

To a solution of methyl 6-formylimidazo[1,2-a]pyridine-8-carboxylate (500 mg, 2.45 mmol) in DMF (5 mL) was added NCS (330 mg, 2.48 mmol). The reaction mixture was stirred for 4 h at 80° C. After completed, the reaction was quenched with water (10 mL) and extracted with DCM (3×30 mL), dried over Na₂SO₄ and concentrated under vacuum. The crude product (600 mg) was obtained and used for next steps without purification. MS: M/e 239. 241 (M+H)⁺.

### Step B: 3-chloro-6-formylimidazo[1,2-a]pyridine-8-carboxylic acid

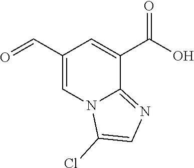 [SMILES: O=Cc1cc(C(=O)O)c2ncc(Cl)n2c1]

To a solution of methyl 3-chloro-6-formylimidazo[1,2-a]pyridine-8-carboxylate (600 mg, crude) in MeOH (20 mL) was added LiOH H₂O (510 mg, 12.14 mmol). The reaction mixture was stirred for 12 h at 25° C. After completed, the reaction mixture was filtrated and the solid was collected and concentrated under vacuum. The product (480 mg) was obtained and used for next steps without purification. MS: M/e 225, 227 (M+H)⁺.

### Step C: 3-chloro-N-(3-((1s, 3s)-3-(cyanomethyl)-1-(4-methyl-4H-1,2,4-triazol-3-yl)cyclobutyl)phenyl)-6-formylimidazo[1,2-a]pyridine-8-carboxamide

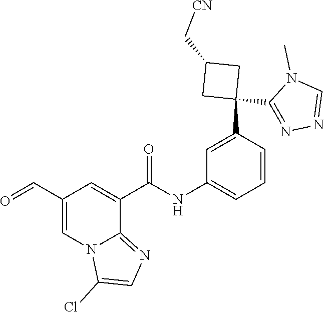 [SMILES: Cn1cnnc1[C@]1(c2cccc(NC(=O)c3cc(C=O)cn4c(Cl)cnc34)c2)C[C@H](CC#N)C1]

To a solution of 3-chloro-6-formylimidazo[1,2-a]pyridine-8-carboxylic acid (480 mg, 2.14 mmol) and 2-((1s, 3s)-3-(3-aminophenyl)-3-(4-methyl-4H-1,2,4-triazol-3-yl)cyclobutyl)acetonitrile (570 mg, 2.13 mmol) in DMF (5 mL) was added HATU (1.2 g, 3.15 mmol) and DIEA (550 mg, 4.26 mmol). The reaction mixture was stirred for 1 h at 25° C. After completed, the reaction was quenched with aq NH₄Cl (20 mL) and extracted with DCM (3×30 mL), dried over Na₂SO₄ and concentrated under vacuum. The residue was purified by column chromatography on silica gel eluting with methanol in dichloromethane (10%) to afford the title compound (600 mg, 59%). MS: M/e 474, 476 (M+H)*; ¹H NMR (400 MHz, DMSO-d₆) δ 11.78-11.72 (m, 1H), 10.18 (s, 1H), 9.47 (d, J=1.6 Hz, 1H), 8.42 (d, J=1.2 Hz, 1H), 8.32 (s, 1H), 8.14 (s, 1H), 7.74-7.68 (m, 2H), 7.46 (t, J=8.0 Hz, 1H), 7.24 (d, J=8.0 Hz, 1H), 3.22 (s, 3H), 2.93-2.86 (m, 2H), 2.76 (d, J=6.8 Hz, 3H), 2.70 (s, 2H) ppm.

### Step D: 3-chloro-N-(3-((1s, 3s)-3-(cyanomethyl)-1-(4-methyl-4H-1,2,4-triazol-3-yl)cyclobutyl)phenyl)-6-((((1-methylcyclopentyl)methyl)amino)methyl)imidazo[1,2-a]pyridine-8-carboxamide

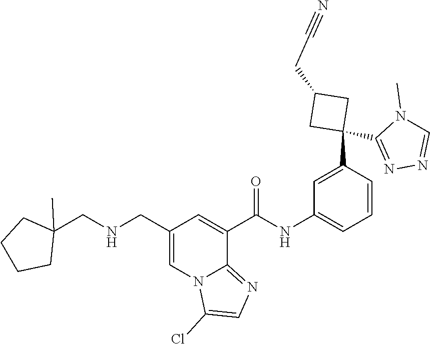 [SMILES: Cn1cnnc1[C@]1(c2cccc(NC(=O)c3cc(CNCC4(C)CCCC4)cn4c(Cl)cnc34)c2)C[C@H](CC#N)C1]

To a mixture of 3-chloro-N-(3-((1s, 3s)-3-(cyanomethyl)-1-(4-methyl-4H-1,2,4-triazol-3-yl)cyclobutyl)phenyl)-6-formylimidazo[1,2-a]pyridine-8-carboxamide (15 mg, 31.65 mol), (1-methylcyclopentyl)methanamine hydrochloride (7.11 mg, 48.48 mol) and TEA (4.8 mg, 47.48 mol) in DCM (1 mL) was added NaBH(OAc)₃ (16.77 mg, 79.13 mol) in one portion at 20° C. The mixture was stirred at 20° C. for 12 hrs. The mixture was poured into water (2 mL) and extracted with DCM (9 mL, 3 mL). The combined organic phase was washed with brine (5 mL), dried over Na₂SO₄, filtered and concentrated to give the residue, which was purified by Prep-HPLC (column=Waters Xbridge Prep OBD C18 (150*40 mm*10 um); mobile phase=water (NH₄HCO₃)-ACN, B %=25%-55%; 8 min) to give the product (5.19 mg, 28.71% yield). MS: M/e 571 (M+1)⁺; ¹H NMR (400 MHz, DMSO-d₆) δ 11.97 (s, 1H), 8.53 (s, 1H), 8.32 (s, 1H), 8.26 (d, J=1.2 Hz, 1H), 7.95 (s, 1H), 7.73 (s, 1H), 7.69 (d, J=8.0 Hz, 1H), 7.44 (t, J=8.0 Hz, 1H), 7.24-7.18 (m, 1H), 3.88 (s, 2H), 3.29 (s, 2H), 3.22 (s, 3H), 2.94-2.83 (m, 2H), 2.76 (d, J=6.8 Hz, 2H), 2.70 (s, 2H), 2.36 (s, 2H), 1.62-1.43 (m, 6H), 1.29-1.20 (m, 2H), 0.97 (s, 3H) ppm.

### Compound A29: 3-chloro-N-(3-((1r, 3r)-3-cyano-1-((4-methyl-4H-1,2,4-triazol-3-yl)methyl)cyclobutyl)phenyl)-6-((((1-methylcyclopentyl)methyl)amino)methyl)imidazo[1,2-a]pyridine-8-carboxamide

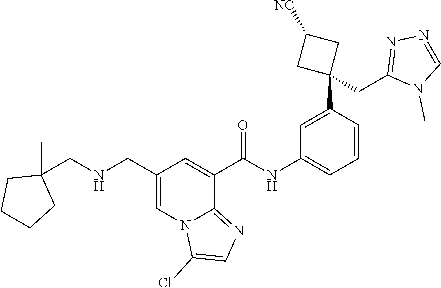 [SMILES: Cn1cnnc1C[C@]1(c2cccc(NC(=O)c3cc(CNCC4(C)CCCC4)cn4c(Cl)cnc34)c2)C[C@H](C#N)C1]

### Step 1: methyl 6-bromo-3-chloroimidazo[1,2-a]pyridine-8-carboxylate

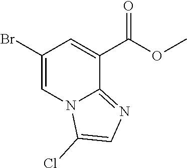

To a solution of methyl 6-bromoimidazo[1,2-a]pyridine-8-carboxylate (10 g, 39.21 mmol) in CH₃CN (100 mL) was added NCS (6.28 g, 47.05 mmol). The resulting mixture was stirred at 60° C. for 16 hrs, then quenched with H₂O and extracted with DCM (300 mL, 100 mL). The combined organic layers were washed with brine (100 mL), and dried over Na₂SO₄. After filtration and concentration, the resulting residue was purified by column chromatography (SiO₂, PE:EA=50/1, 1/1) to give the product (11 g, 96.91% yield). MS: M/e 291 (M+1)⁺; ¹H NMR (400 MHz, DMSO-d₆) δ 9.00 (d, J=1.6 Hz, 1H), 8.12-8.08 (m, 1H), 7.99 (s, 1H), 3.94 (s, 3H) ppm.

### Step 2: methyl 3-chloro-6-vinylimidazo[1,2-a]pyridine-8-carboxylate

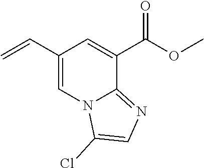 [SMILES: C=Cc1cc(C(=O)OC)c2ncc(Cl)n2c1]

A mixture of methyl 6-bromo-3-chloroimidazo[1,2-a]pyridine-8-carboxylate (11 g, 37.99 mmol), potassium trifluoro(vinyl)borate (15.27 g, 113.98 mmol), Pd(dppf)Cl₂ (2.78 g, 3.8 mmol) and K₂CO₃ (15.75 g, 113.98 mmol) in Dioxane (90 mL) and H₂O (30 mL) was degassed and purged with N₂ for 3 times, and then the mixture was stirred at 100° C. for 12 hrs under N₂ atmosphere. The reaction mixture was added addition H₂O (50 mL) at 20° C., and then extracted with DCM (120 mL, 40 mL). The combined organic layers were washed with brine (60 mL, 30 mL), dried over Na₂SO₄, filtered and concentrated under reduced pressure to give a residue. The resulting residue was purified by column chromatography (SiO₂, Petroleum ether/Ethyl acetate=100/1 to 0/1) to give product (5.5 g, yield 61.18%). MS: M/e 237 (M+1)⁺; ¹H NMR (400 MHz, DMSO-d₆) δ 8.64 (s, 1H), 8.16 (s, 1H), 7.77 (s, 1H), 6.99-6.85 (m, 1H), 6.00 (d, J=17.6 Hz, 1H), 5.43 (d, J=10.8 Hz, 1H), 4.01-3.87 (m, 3H) ppm.

### Step 3: lithium 3-chloro-6-vinylimidazo[1,2-a]pyridine-8-carboxylate

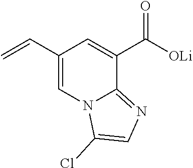

To a solution of methyl 3-chloro-6-vinylimidazo[1,2-a]pyridine-8-carboxylate (1 g, 4.23 mmol) in MeOH/THF/H₂O (5 mL/5 mL/5 mL) was added LiOH·H₂O (212.78 mg, 5.07 mmol). The mixture was stirred for 1 hr at 30° C. The mixture was filtered and concentrated to give the crude product (1 g, crude), which was used to the next step without purification. MS: M/e 223 (M+1)⁺; ¹H NMR (400 MHz, DMSO-d₆) δ 8.48-8.44 (m, 1H), 8.23-8.16 (m, 1H), 7.68 (s, 1H), 6.98-6.87 (m, 1H), 5.93 (d, J=17.6 Hz, 1H), 5.41 (d, J=10.8 Hz, 1H) ppm

### Step 4: 3-chloro-N-(3-((1r, 3r)-3-cyano-1-((4-methyl-4H-1,2,4-triazol-3-yl)methyl)cyclobutyl)phenyl)-6-vinylimidazo[1,2-a]pyridine-8-carboxamide

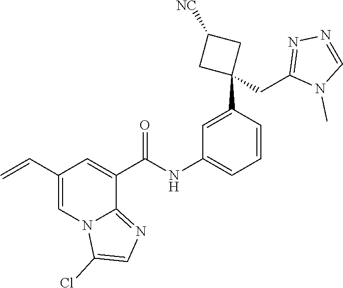 [SMILES: C=Cc1cc(C(=O)Nc2cccc([C@]3(Cc4nncn4C)C[C@@H](C#N)C3)c2)c2ncc(Cl)n2c1]

To a solution of lithium 3-chloro-6-vinylimidazo[1,2-a]pyridine-8-carboxylate (250 mg, 929.73 mol) in DMF (3 mL) was added DIEA (240.23 mg, 1.86 mmol) and HATU (424.21 mg, 1.12 mmol). The mixture was stirred for 5 mins at RT, then (1r, 3r)-3-(3-aminophenyl)-3-((4-methyl-4H-1,2,4-triazol-3-yl)methyl)cyclobutanecarbonitrile (248.54 mg, 929.73 mol) was added. The mixture was stirred for 12 hrs at RT. The mixture was poured into water (1 mL) and extracted with DCM (2 mL, 1 mL). The combined organic phase was filtered and concentrated to give the residue, The resulting residue was purified by column chromatography (SiO₂, Petroleum ether/Ethyl acetate=100/1 to 0/1) to give product (230 mg, yield 52.41%). MS: M/e 472 (M+1)^{+ 1}H NMR (400 MHz, DMSO-d₆) δ 11.90 (s, 1H), 8.72 (s, 1H), 8.41 (s, 1H), 8.18 (s, 1H), 7.96 (s, 1H), 7.70-7.63 (m, 1H), 7.47-7.42 (m, 1H), 7.32 (t, J=8.0 Hz, 1H), 7.05-6.93 (m, 1H), 6.76 (d, J=7.6 Hz, 1H), 6.07 (d, J=17.6 Hz, 1H), 5.49 (d, J=11.2 Hz, 1H), 3.30-3.20 (m, 2H), 2.93-2.82 (m, 4H), 2.81 (s, 3H), 1.75 (s, 1H) ppm.

### Step 5: 3-chloro-N-(3-((1r, 3r)-3-cyano-1-((4-methyl-4H-1,2,4-triazol-3-yl)methyl)cyclobutyl)phenyl)-6-formylimidazo[1,2-a]pyridine-8-carboxamide

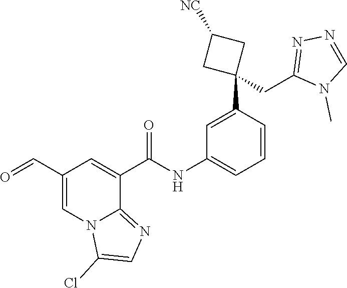 [SMILES: Cn1cnnc1C[C@]1(c2cccc(NC(=O)c3cc(C=O)cn4c(Cl)cnc34)c2)C[C@H](C#N)C1]

To a mixture of 3-chloro-N-(3-((1r, 3r)-3-cyano-1-((4-methyl-4H-1,2,4-triazol-3-yl)methyl)cyclobutyl)phenyl)-6-vinylimidazo[1,2-a]pyridine-8-carboxamide (230 mg, 478.35 mol) and K₂OsO₄·H₂O (17.96 mg, 48.73 mol) in Dioxane (10 mL) and H₂O (2 mL) was added NaIO₄ (416.96 mg, 1.95 mmol) in one portion at 20° C. The mixture was stirred at 20° C. and stirred for 16 hours. The reaction mixture was quenched by addition H₂O 10 mL at 20° C., and then extracted with DCM (30 mL, 10 mL). The combined organic layers were washed with brine (30 mL, 10 mL), dried over Na₂SO₄, filtered and concentrated under reduced pressure to give the crude product, The combined organic phase was filtered and concentrated to give the residue, which was purified by Prep-HPLC (column=Waters Xbridge Prep OBD C18 (150*40 mm*10 um); mobile phase=water (NH4HCO₃)-ACN, B %=25%-55%; 8 min) to give the product (20 mg, 8.65% yield). MS: M/e 474 (M+1); ¹H NMR (400 MHz, DMSO-d₆) δ 11.79-11.73 (m, 1H), 10.18 (s, 1H), 9.49 (d, J=1.6 Hz, 1H), 8.43 (d, J=1.2 Hz, 1H), 8.18 (s, 1H), 8.14 (s, 1H), 7.75-7.68 (m, 1H), 7.38 (s, 1H), 7.32 (s, 1H), 6.77 (d, J=8.0 Hz, 1H), 3.31 (s, 2H), 3.29-3.24 (m, 1H), 2.92-2.79 (m, 7H) ppm.

### Step 6: 3-chloro-N-(3-((1r, 3r)-3-cyano-1-((4-methyl-4H-1,2,4-triazol-3-yl)methyl)cyclobutyl)phenyl)-6-((((1-methylcyclopentyl)methyl)amino)methyl)imidazo[1,2-a]pyridine-8-carboxamide

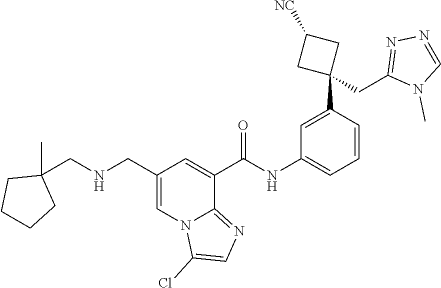 [SMILES: Cn1cnnc1C[C@]1(c2cccc(NC(=O)c3cc(CNCC4(C)CCCC4)cn4c(Cl)cnc34)c2)C[C@H](C#N)C1]

To a mixture of 3-chloro-N-(3-((1r, 3r)-3-cyano-1-((4-methyl-4H-1,2,4-triazol-3-yl)methyl)cyclobutyl)phenyl)-6-formylimidazo[1,2-a]pyridine-8-carboxamide (20 mg, 42.2 mol), (1-methylcyclopentyl)methanamine hydrochloride (9.47 mg, 63.3 mol) and TEA (6.41 mg, 63.3 mol) in DCM (1 mL) was added NaBH(OAc)₃ (22.36 mg, 105.5 mol) in one portion at 20° C. The mixture was stirred at 20° C. for 12 hrs. The mixture was poured into water (2 mL) and extracted with DCM (9 mL, 3 mL). The combined organic phase was washed with brine (5 mL), dried over Na₂SO₄, filtered and concentrated to give the residue, which was purified by Prep-HPLC (column=Waters Xbridge Prep OBD C18 (150*40 mm*10 um); mobile phase=water (NH₄HCO₃)-ACN, B %=25%-55%; 8 min) to give the product (4.45 mg, 18.46% yield). MS: M/e 571 (M+1)⁺; ¹H NMR (400 MHz, DMSO-d₆) δ 11.97 (s, 1H), 8.54 (s, 1H), 8.27 (d, J=1.2 Hz, 1H), 8.17 (s, 1H), 7.95 (s, 1H), 7.68 (d, J=8.4 Hz, 1H), 7.40 (s, 1H), 7.31 (t, J=8.0 Hz, 1H), 6.78-6.72 (m, 1H), 3.88 (s, 2H), 3.29 (s, 2H), 3.27-3.22 (m, 1H), 2.93-2.83 (m, 4H), 2.81 (s, 3H), 2.36 (s, 2H), 1.63-1.45 (m, 6H), 1.30-1.20 (m, 3H), 0.98 (s, 3H) ppm.

### Compound A30: N-(3-((1s, 3s)-3-(cyanomethyl)-1-(4-methyl-4H-1,2,4-triazol-3-yl)cyclobutyl)phenyl)-6-((((1-methylcyclobutyl)methyl)amino)methyl)imidazo[1,2-a]pyridine-8-carboxamide

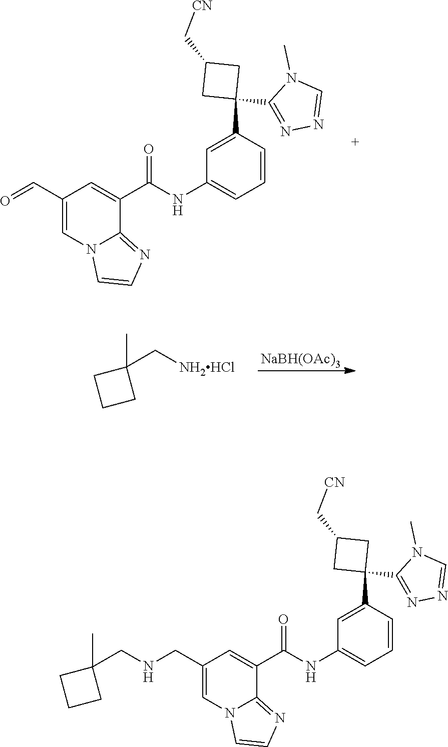

The compound A30 was synthesized starting from the corresponding starting materials according the similar procedures described as those of Compound A23 to get the product (23 mg, 26%). H NMR (400 MHz, CD₃OD) δ 8.82 (s, 1H), 8.33 (s, 1H), 8.32 (s, 1H), 8.05 (s, 1H), 7.93 (s, 1H), 7.79 (s, 1H), 7.71 (d, J=8.0 Hz, 1H), 7.47 (t, J=8.0 Hz, 1H), 7.25 (d, J=8.0 Hz, 1H), 4.27 (s, 2H), 3.33 (s, 3H), 3.07-3.01 (m, 2H), 3.00 (s, 2H), 2.97-2.84 (m, 3H), 2.67 (d, J=8.0 Hz, 2H), 2.00-1.95 (m, 3H), 1.91-1.76 (m, 3H), 1.26 (s, 3H) ppm. MS: M/e 523 (M+1)+

### Compound A31: N-(3-((1s,3R)-3-(cyanomethyl)-1-(4-methyl-4H-1,2,4-triazol-3-yl)cyclobutyl)phenyl)-6-((((S)-1-cyclopentylethyl)amino)methyl)imidazo[1,2-a]pyridine-8-carboxamide

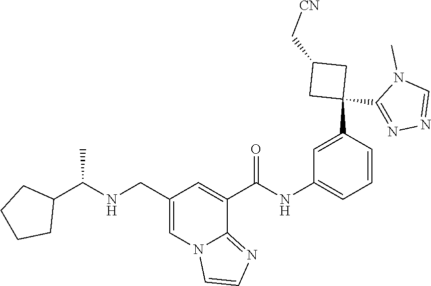 [SMILES: C[C@H](NCc1cc(C(=O)Nc2cccc([C@]3(c4nncn4C)C[C@@H](CC#N)C3)c2)c2nccn2c1)C1CCCC1]

The compound A31 was synthesized starting from the corresponding starting materials according the similar procedures described as those of Compound A23 to give the product (20.50 mg, 33.57% yield). MS: M/e 537 (M+1)⁺. ¹H NMR (400 MHz, DMSO-d₆) δ=12.42 (s, 1H), 8.73 (s, 1H), 8.32 (s, 1H), 8.18 (d, J=1.5 Hz, 1H), 8.14 (d, J=1.3 Hz, 1H), 7.78 (d, J=1.3 Hz, 1H), 7.75 (s, 1H), 7.72-7.65 (m, 1H), 7.44 (t, J=7.9 Hz, 1H), 7.20 (d, J=8.0 Hz, 1H), 3.93-3.80 (m, 1H), 3.78-3.68 (m, 1H), 3.22 (s, 3H), 2.89 (br d, J=2.8 Hz, 2H), 2.81-2.67 (m, 5H), 2.44-2.36 (m, 1H), 2.04 (br d, J=1.8 Hz, 1H), 1.87-1.70 (m, 2H), 1.69-1.36 (m, 5H), 1.30-1.11 (m, 2H), 1.01 (d, J=6.1 Hz, 3H) ppm.

### Compound A32: N-(3-((1s, 3s)-3-(cyanomethyl)-1-(4-methyl-4H-1,2,4-triazol-3-yl)cyclobutyl)phenyl)-6-((neopentylamino)methyl)imidazo[1,2-a]pyridine-8-carboxamide

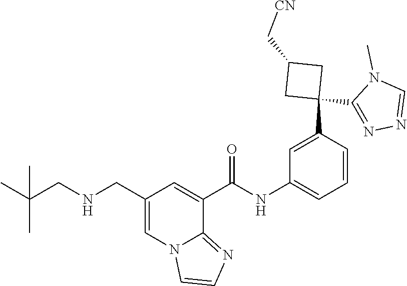 [SMILES: Cn1cnnc1[C@]1(c2cccc(NC(=O)c3cc(CNCC(C)(C)C)cn4ccnc34)c2)C[C@H](CC#N)C1]

The compound A32 was synthesized starting from the corresponding starting materials according the similar procedures described as those of Compound A23 to give the product (6.13 mg). MS: M/e 511 (M+1)⁺; H NMR (400 MHz, DMSO-d₆) δ=12.44 (s, 1H), 8.72 (s, 1H), 8.33 (s, 1H), 8.20 (d, J=4 Hz, 1H), 8.15 (d, J=4 Hz, 1H), 7.79 (d, J=4 Hz, 1H), 7.76 (s, 1H), 7.70 (d, J=8 Hz, 1H), 7.45 (t, J=8 Hz, 1H), 7.21 (d, J=8 Hz, 1H), 3.80 (s, 2H), 3.23 (s, 3H), 2.90 (m, 2H), 2.81-2.69 (m, 5H), 2.54-2.53 (m, 1H), 2.27 (s, 2H), 0.88 (s, 9H) ppm.

### Compound A33: N-(3-((1r,3S)-3-cyano-1-((4-methyl-4H-1,2,4-triazol-3-yl)methyl)cyclobutyl)phenyl)-6-((((S)-1-cyclopentylethyl)amino)methyl)imidazo[1,2-a]pyridine-8-carboxamide

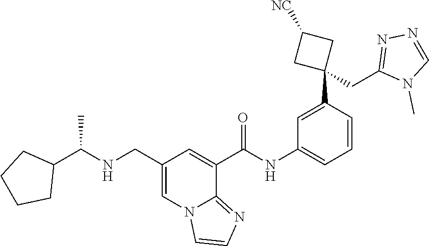 [SMILES: C[C@H](NCc1cc(C(=O)Nc2cccc([C@]3(Cc4nncn4C)C[C@@H](C#N)C3)c2)c2nccn2c1)C1CCCC1]

The compound A33 was synthesized starting from the corresponding starting materials according the similar procedures described as those of Compound A24 to give the product (7.12 mg, 23.32% yield). MS: M/e 537 (M+1)⁺; H NMR (400 MHz, DMSO-d₆) δ=12.43-12.38 (m, 1H), 8.73 (s, 1H), 8.20-8.15 (m, 2H), 8.15-8.14 (m, 1H), 7.78 (d, J=1.2 Hz, 1H), 7.71-7.67 (m, 1H), 7.41 (s, 1H), 7.31 (s, 1H), 6.73 (d, J=8.2 Hz, 1H), 3.92-3.84 (m, 1H), 3.78-3.69 (m, 1H), 3.32-3.23 (m, 4H), 2.94-2.83 (m, 4H), 2.80 (s, 3H), 2.44-2.36 (m, 1H), 1.84-1.73 (m, 2H), 1.67-1.58 (m, 1H), 1.56-1.42 (m, 4H), 1.27-1.13 (m, 2H), 1.04-0.98 (m, 3H) ppm.

### Compound A34: 3-chloro-N-(3-((1s,3R)-3-(cyanomethyl)-1-(4-methyl-4H-1,2,4-triazol-3-yl)cyclobutyl)phenyl)-6-((((S)-1-cyclopentylethyl)amino)methyl)imidazo[1,2-a]pyridine-8-carboxamide

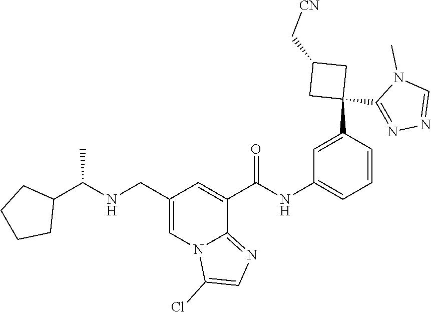 [SMILES: C[C@H](NCc1cc(C(=O)Nc2cccc([C@]3(c4nncn4C)C[C@@H](CC#N)C3)c2)c2ncc(Cl)n2c1)C1CCCC1]

The compound A34 was synthesized starting from the corresponding starting materials according to the similar procedures described as those of Compound A28 to afford the title compound (45 mg, 54%). ¹H NMR (400 MHz, CD₃OD) δ 8.54 (s, 1H), 8.33 (s, 1H), 8.26 (s, 1H), 7.99-7.92 (m, 2H), 7.61 (d, J=8.0 Hz, 1H), 7.46 (t, J=8.0 Hz, 1H), 7.20 (d, J=7.4 Hz, 1H), 3.93 (d, J=13.8 Hz, 1H), 3.79 (d, J=13.8 Hz, 1H), 3.34 (s, 3H), 3.08-2.82 (m, 5H), 2.67 (d, J=6.0 Hz, 2H), 2.53-2.42 (m, 1H), 1.92-1.68 (m, 3H), 1.65-1.48 (m, 4H), 1.29-1.14 (m, 2H), 1.11 (d, J=6.4 Hz, 3H) ppm. MS: M/e 571/573 (M+H)⁺.

### Compound A35: 3-chloro-N-(3-((1s, 3s)-3-(cyanomethyl)-1-(4-methyl-4H-1,2,4-triazol-3-yl)cyclobutyl)phenyl)-6-((((1-methylcyclobutyl)methyl)amino)methyl)imidazo[1,2-a]pyridine-8-carboxamide

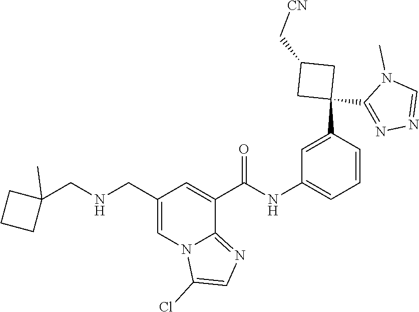 [SMILES: Cn1cnnc1[C@]1(c2cccc(NC(=O)c3cc(CNCC4(C)CCC4)cn4c(Cl)cnc34)c2)C[C@H](CC#N)C1]

The compound A35 was synthesized starting from the corresponding starting materials according the similar procedures described as those of Compound A28 to afford the title compound (35 mg, 59%). ¹H NMR (400 MHz, CD₃OD) δ 8.52 (s, 1H), 8.34 (s, 1H), 8.26 (d, J=6.8 Hz, 1H), 7.98-7.90 (m, 2H), 7.60 (d, J=7.8 Hz, 1H), 7.45 (t, J=7.8 Hz, 1H), 7.19 (d, J=7.8 Hz, 1H), 3.86 (d, J=2.0 Hz, 2H), 3.34 (s, 3H), 3.08-2.82 (m, 5H), 2.67 (d, J=6.0 Hz, 2H), 2.56 (s, 2H), 1.97-1.76 (m, 4H), 1.76-1.64 (m, 2H), 1.17 (s, 3H) ppm. MS: M/e 557, 559 (M+H)⁺.

### Compound A36: N-(3-((1s, 3s)-3-(cyanomethyl)-1-(4-methyl-4H-1,2,4-triazol-3-yl)cyclobutyl)phenyl)-6-(((1-methylcyclobutyl)amino)methyl)imidazo[1,2-a]pyridine-8-carboxamide compound with N-(3-((1s, 3s)-3-(cyanomethyl)-1-(4-methyl-4H-1,2,4-triazol-3-yl)cyclobutyl)phenyl)-6-formylimidazo[1,2-a]pyridine-8-carboxamide (1:1)

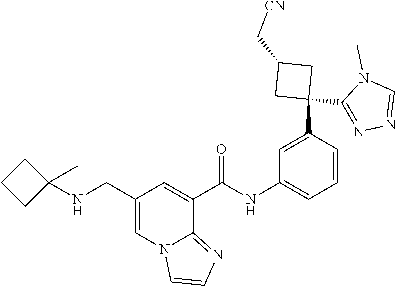 [SMILES: Cn1cnnc1[C@]1(c2cccc(NC(=O)c3cc(CNC4(C)CCC4)cn4ccnc34)c2)C[C@H](CC#N)C1]

The compound A36 was synthesized starting from the corresponding starting materials according the similar procedures described as those of Compound A23 to give the product (59.62 mg, 117.22 umol, 51.51% yield). MS: M/e 509 (M+1)⁺; ¹H NMR (400 MHz, DMSO-d₆) δ ppm 12.42 (s, 1H), 8.75 (d, J=1.38 Hz, 1H), 8.32 (s, 1H), 8.15 (dd, J=6.50, 1.38 Hz, 2H), 7.73-7.80 (m, 2H), 7.69 (dd, J=8.07, 1.19 Hz, 1H), 7.44 (t, J=7.88 Hz, 1H), 7.20 (d, J=8.50 Hz, 1H), 3.71 (s, 2H), 3.22 (s, 3H), 2.84-2.98 (m, 2H), 2.62-2.83 (m, 5H), 2.35-2.46 (m, 1H), 1.92-2.05 (m, 2H), 1.59-1.79 (m, 4H), 1.25 (s, 3H) ppm.

### Compound A37: N-(3-((1s, 3s)-3-(cyanomethyl)-1-(4-methyl-4H-1,2,4-triazol-3-yl)cyclobutyl)phenyl)-6-((((1-fluorocyclobutyl)methyl)amino)methyl)imidazo[1,2-a]pyridine-8-carboxamide

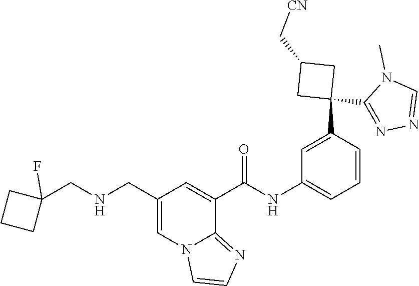 [SMILES: Cn1cnnc1[C@]1(c2cccc(NC(=O)c3cc(CNCC4(F)CCC4)cn4ccnc34)c2)C[C@H](CC#N)C1]

The compound A37 was synthesized starting from the corresponding starting materials according the similar procedures described as those of Compound A23 to give product (26.58 mg, yield: 31.4%). MS: M/e 527 (M+1)⁺, ¹H NMR (400 MHz, DMSO-d₆) δ=12.42 (s, 1H), 8.73 (s, 1H), 8.32 (s, 1H), 8.19 (s, 1H), 8.15 (s, 1H), 7.79 (s, 1H), 7.75 (s, 1H), 7.70 (br d, J=8.0 Hz, 1H), 7.44 (t, J=7.8 Hz, 1H), 7.20 (d, J=7.6 Hz, 1H), 3.85 (br s, 2H), 3.22 (s, 3H), 2.94-2.84 (m, 2H), 2.84-2.72 (m, 5H), 2.71 (br s, 2H), 2.23-2.09 (m, 4H), 1.80-1.66 (m, 1H), 1.50-1.35 (m, 1H) ppm.

### Compound A38: N-(3-((1s,3R)-3-(cyanomethyl)-1-(4-methyl-4H-1,2,4-triazol-3-yl)cyclobutyl)phenyl)-6-((((S)-1-cyclobutylethyl)amino)methyl)imidazo[1,2-a]pyridine-8-carboxamide

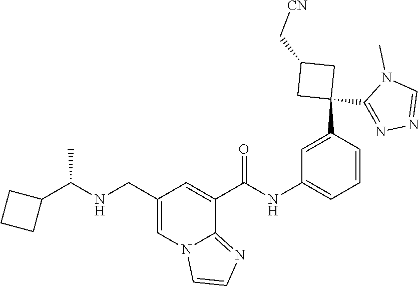 [SMILES: C[C@H](NCc1cc(C(=O)Nc2cccc([C@]3(c4nncn4C)C[C@@H](CC#N)C3)c2)c2nccn2c1)C1CCC1]

The compound A38 was synthesized starting from the corresponding starting materials according the similar procedures described as those of Compound A23 to give product (88.6 mg, yield: 62.1%). MS: M/e 523 (M+1)⁺; ¹H NMR (400 MHz, DMSO-d₆) δ=12.42 (s, 1H), 8.72 (s, 1H), 8.32 (s, 1H), 8.16 (dd, J=1.4, 11.8 Hz, 2H), 7.78 (d, J=1.2 Hz, 1H), 7.74 (s, 1H), 7.70 (br d, J=8.0 Hz, 1H), 7.44 (t, J=7.8 Hz, 1H), 7.20 (br d, J=7.8 Hz, 1H), 3.81 (s, 1H), 3.77-3.69 (m, 1H), 3.22 (s, 3H), 2.89 (br d, J=2.6 Hz, 2H), 2.76 (br d, J=6.0 Hz, 3H), 2.71 (br s, 2H), 2.24-2.14 (m, 1H), 2.05-1.93 (m, 2H), 1.92-1.84 (m, 1H), 1.82-1.60 (m, 4H), 0.91 (d, J=6.0 Hz, 3H) ppm.

### Compound A39: N-(3-((1s, 3s)-3-methyl-1-(4-methyl-4H-1,2,4-triazol-3-yl)cyclobutyl)phenyl)-6-(((1-methylcyclobutyl)amino)methyl)imidazo[1,2-a]pyridine-8-carboxamide

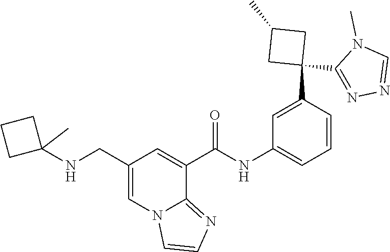 [SMILES: Cn1cnnc1[C@]1(c2cccc(NC(=O)c3cc(CNC4(C)CCC4)cn4ccnc34)c2)C[C@H](C)C1]

The compound A39 was synthesized starting from the corresponding starting materials according the similar procedures described as those of Compound A22 to give product (11.36 mg, 24.34% yield). MS: M/e 484 (M+1)⁺. ¹H NMR (400 MHz, DMSO-d₆) δ=12.44-12.36 (m, 1H), 8.77-8.69 (m, 1H), 8.29 (s, 1H), 8.17-8.10 (m, 2H), 7.81-7.73 (m, 2H), 7.69-7.62 (m, 1H), 7.48-7.36 (m, 1H), 7.15 (d, J=7.6 Hz, 1H), 3.73-3.67 (m, 2H), 3.20 (s, 3H), 2.91-2.78 (m, 2H), 2.54 (d, J=6.4 Hz, 3H), 2.06-1.93 (m, 2H), 1.78-1.63 (m, 4H), 1.29-1.21 (m, 3H), 1.12-1.05 (m, 3H) ppm.

### Compound A40: 6-((((S)-1-cyclopentylethyl)amino)methyl)-N-(3-((1s,3R)-3-methyl-1-(4-methyl-4H-1,2,4-triazol-3-yl)cyclobutyl)phenyl)imidazo[1,2-a]pyridine-8-carboxamide

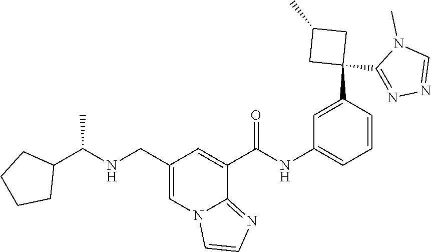 [SMILES: C[C@H](NCc1cc(C(=O)Nc2cccc([C@]3(c4nncn4C)C[C@@H](C)C3)c2)c2nccn2c1)C1CCCC1]

The compound A40 was synthesized starting from the corresponding starting materials according the similar procedures described as those of Compound A22 to give the product (2.58 mg, 5.22% yield). MS: M/e 512 (M+1)⁺. ¹H NMR (400 MHz, DMSO-d₆) δ=12.41 (s, 1H), 8.72 (s, 1H), 8.30 (s, 1H), 8.17 (d, J=1.2 Hz, 1H), 8.13 (d, J=1.2 Hz, 1H), 7.77 (d, J=1.2 Hz, 1H), 7.75-7.72 (m, 1H), 7.67 (d, J=8.0 Hz, 1H), 7.45-7.39 (m, 1H), 7.39-7.38 (m, 1H), 7.15 (d, J=8.0 Hz, 1H), 3.90-3.82 (m, 1H), 3.77-3.69 (m, 1H), 3.20 (s, 3H), 2.84 (d, J=3.2 Hz, 2H), 2.60-2.52 (m, 3H), 2.45-2.35 (m, 1H), 1.82-1.72 (m, 2H), 1.69-1.56 (m, 1H), 1.56-1.41 (m, 4H), 1.29-1.13 (m, 2H), 1.11-1.06 (m, 3H), 1.00 (d, J=6.4 Hz, 3H) ppm.

### Compound A41: 3-chloro-N-(3-((1s, 3s)-3-(cyanomethyl)-1-(4-methyl-4H-1,2,4-triazol-3-yl)cyclobutyl)phenyl)-6-(((1-methylcyclopropyl)amino)methyl)imidazo[1,2-a]pyridine-8-carboxamide

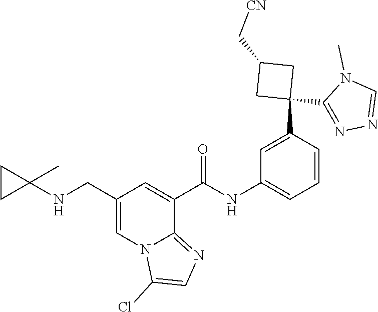 [SMILES: Cn1cnnc1[C@]1(c2cccc(NC(=O)c3cc(CNC4(C)CC4)cn4c(Cl)cnc34)c2)C[C@H](CC#N)C1]

To a solution of 1-methylcyclopropan-1-amine hydrochloride (17.03 mg, 158.26 umol) in DCE (4 mL) was added TEA (16.01 mg, 158.26 umol), 3-chloro-N-(3-((1s, 3s)-3-(cyanomethyl)-1-(4-methyl-4H-1,2,4-triazol-3-yl)cyclobutyl)phenyl)-6-formylimidazo[1,2-a]pyridine-8-carboxamide (50 mg, 105.50 umol) and NaBH(OAc)₃ (67.08 mg, 316.51 umol). The mixture was stirred at 25° C. for 16 hrs. The reaction mixture was diluted with H₂O (1 mL) and filtered and concentrated under reduced pressure to give a residue, which was purified by Prep-HPLC (column: Waters Xbridge BEH C18 100*30 mm*10 um; mobile phase: [water (NH₄HCO₃)-ACN]; B %: 35%-55%, 8 min) to give the product (7.13 mg, 12.77% yield). MS: M/e 529 (M+1)⁺; H NMR (400 MHz, DMSO-d₆) δ=11.96 (s, 1H), 8.51 (d, J=1.2 Hz, 1H), 8.32 (s, 1H), 8.19 (d, J=1.6 Hz, 1H), 7.95 (s, 1H), 7.74 (s, 1H), 7.70-7.62 (m, 1H), 7.44 (t, J=8.0 Hz, 1H), 7.21 (d, J=8.0 Hz, 1H), 3.90 (s, 2H), 3.21 (s, 3H), 2.95-2.82 (m, 2H), 2.76 (br d, J=8.0 Hz, 3H), 2.70 (br s, 2H), 2.61 (br s, 1H), 1.27 (s, 3H), 0.60-0.47 (m, 2H), 0.38-0.27 (m, 2H) ppm.

### Compound A42: 6-(((cyclopentylmethyl)amino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

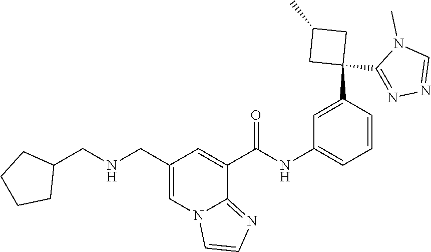 [SMILES: Cn1cnnc1[C@]1(c2cccc(NC(=O)c3cc(CNCC4CCCC4)cn4ccnc34)c2)C[C@H](C)C1]

The compound A42 was synthesized starting from the corresponding starting materials according the similar procedures described as those of Compound A22 to give the product (13.91 mg, 28.96% yield). MS: M/e 498 (M+1)⁺. ¹H NMR (400 MHz, DMSO-d₆) δ=12.44-12.39 (m, 1H), 8.71 (d, J=0.4 Hz, 1H), 8.33-8.27 (m, 1H), 8.18-8.11 (m, 2H), 7.79-7.76 (m, 1H), 7.75-7.72 (m, 1H), 7.69-7.65 (m, 1H), 7.42 (t, J=8.0 Hz, 1H), 7.15 (d, J=8.0 Hz, 1H), 3.77 (s, 2H), 3.20 (s, 3H), 2.90-2.78 (m, 2H), 2.55 (d, J=6.4 Hz, 3H), 2.43 (d, J=7.2 Hz, 2H), 2.03-1.93 (m, 1H), 1.75-1.64 (m, 2H), 1.59-1.41 (m, 4H), 1.23-1.12 (m, 2H), 1.12-1.06 (m, 3H) ppm.

### Compound A43: N-(3-((1s, 3s)-3-(cyanomethyl)-1-(4-methyl-4H-1,2,4-triazol-3-yl)cyclobutyl)phenyl)-6-(((cyclobutylmethyl)amino)methyl)imidazo[1,2-a]pyridine-8-carboxamide

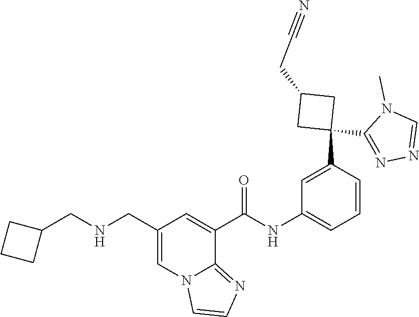 [SMILES: Cn1cnnc1[C@]1(c2cccc(NC(=O)c3cc(CNCC4CCC4)cn4ccnc34)c2)C[C@H](CC#N)C1]

The compound A43 was synthesized starting from the corresponding starting materials according the similar procedures described as those of Compound A23 to give the product (30.83 mg). MS: M/e 509 (M+1)⁺; ¹H NMR (400 MHz, DMSO-d₆) δ=12.42 (s, 1H), 8.71 (s, 1H), 8.32 (s, 1H), 8.14 (d, J=1.4, 4.0 Hz, 2H), 7.78 (d, J=2.0 Hz, 1H), 7.74 (s, 1H), 7.69 (d, J=8.0 Hz, 1H), 7.44 (t, J=8.0 Hz, 1H), 7.20 (d, J=8.0 Hz, 1H), 3.76 (s, 2H), 3.22 (s, 3H), 2.91-2.86 (m, 2H), 2.76 (d, J=8.0 Hz, 3H), 2.73-2.68 (m, 2H), 2.54 (d, J=8.0 Hz, 2H), 2.46-2.37 (m, 1H), 2.03-1.93 (m, 2H), 1.87-1.75 (m, 2H), 1.70-1.57 (m, 2H) ppm.

### Compound A44: 3-fluoro-N-(3-((1s, 3s)-3-methyl-1-(4-methyl-4H-1,2,4-triazol-3-yl)cyclobutyl)phenyl)-6-(((1-methylcyclobutyl)amino)methyl)imidazo[1,2-a]pyridine-8-carboxamide

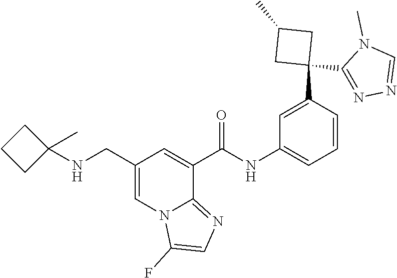 [SMILES: Cn1cnnc1[C@]1(c2cccc(NC(=O)c3cc(CNC4(C)CCC4)cn4c(F)cnc34)c2)C[C@H](C)C1]

The compound A44 was synthesized starting from the corresponding starting materials according the similar procedures described as those of Compound A19, (5.23 mg, 10.43 umol, 23.61% yield) was obtained. MS: M/e 502 (M+1)⁺; ¹H NMR (400 MHz, DMSO-d₆) δ ppm 11.94 (s, 1H), 8.49 (s, 1H), 8.29 (s, 1H), 8.17 (d, J=0.88 Hz, 1H), 7.71 (s, 1H), 7.59-7.68 (m, 2H), 7.42 (t, J=7.88 Hz, 1H), 7.16 (d, J=7.88 Hz, 1H), 3.75 (s, 2H), 3.19 (s, 3H), 2.78-2.91 (m, 2H), 2.54-2.54 (m, 1H), 2.54 (d, J=6.50 Hz, 3H), 1.92-2.05 (m, 2H), 1.63-1.79 (m, 4H), 1.26 (s, 3H), 1.08 (d, J=5.12 Hz, 3H) ppm.

### Compound A45: 3-fluoro-N-(3-((1s, 3s)-3-methyl-1-(4-methyl-4H-1,2,4-triazol-3-yl)cyclobutyl)phenyl)-6-(((1-methylcyclopropyl)amino)methyl)imidazo[1,2-a]pyridine-8-carboxamide

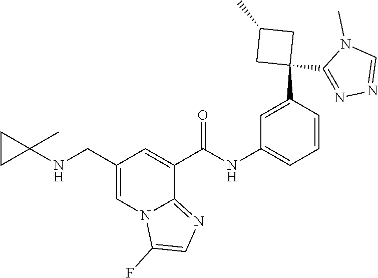 [SMILES: Cn1cnnc1[C@]1(c2cccc(NC(=O)c3cc(CNC4(C)CC4)cn4c(F)cnc34)c2)C[C@H](C)C1]

The compound A45 was synthesized starting from the corresponding starting materials according the similar procedures described as those of Compound A19 (4.10 mg, 8.41 μmol, 19.04% yield) was obtained. MS: M/e 488 (M+1)⁺; ¹H NMR (400 MHz, DMSO-d₆) δ ppm 11.93 (s, 1H), 8.46 (s, 1H), 8.29 (s, 1H), 8.12 (d, J=1.38 Hz, 1H), 7.67-7.74 (m, 1H), 7.59-7.67 (m, 2H), 7.42 (t, J=7.94 Hz, 1H), 7.16 (d, J=8.38 Hz, 1H), 3.86 (s, 2H), 3.19 (s, 3H), 2.75-2.91 (m, 2H), 2.50-2.61 (m, 3H), 1.27 (s, 3H), 1.09 (d, J=5.38 Hz, 3H), 0.53 (s, 2H), 0.29-0.40 (m, 2H) ppm.

### Compound A46: 6-(((3R,4S)-4-fluoro-3-methylpiperidin-1-yl)methyl)-3-methyl-N-(3-((1s,3S)-3-methyl-1-(4-methyl-4H-1,2,4-triazol-3-yl)cyclobutyl)phenyl)imidazo[1,2-a]pyridine-8-carboxamide

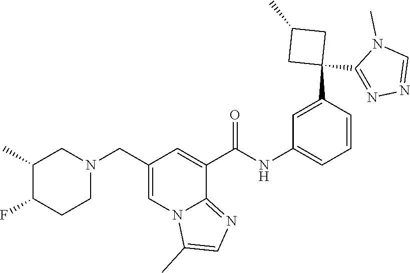 [SMILES: Cc1cnc2c(C(=O)Nc3cccc([C@]4(c5nncn5C)C[C@@H](C)C4)c3)cc(CN3CC[C@H](F)[C@H](C)C3)cn12]

### Step 1: 6-formyl-3-methylimidazo[1,2-a]pyridine-8-carboxylic acid

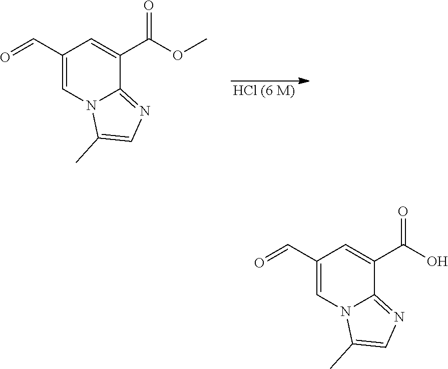 [SMILES: COC(=O)c1cc(C=O)cn2c(C)cnc12 ; "HCl (6 M)" ; Cc1cnc2c(C(=O)O)cc(C=O)cn12]

A solution of methyl 6-formyl-3-methylimidazo[1,2-a]pyridine-8-carboxylate (100 mg, 0.435 mmol, 1 equiv, 95%) in HCl (5.00 mL, 59.195 mmol, 136.08 equiv, 36%) and H₂O (5.00 mL, 277.321 mmol, 637.52 equiv, 100%) was stirred for 2 hrs at 90° C. The mixture was allowed to cool down to room temperature. The resulting mixture was concentrated under vacuum. The residue was purified by reversed-phase flash chromatography with the following conditions: column, C18 silica gel; mobile phase, MeCN in Water (0.1% FA), 0% to 10% gradient in 10 min; detector, UV 254 nm. This resulted in 6-formyl-3-methylimidazo[1,2-a]pyridine-8-carboxylic acid (50 mg, 50.57%). MS: M/e 205 (M+1)⁺.

### Step 2: 6-formyl-3-methyl-N-(3-((1s, 3s)-3-methyl-1-(4-methyl-4H-1,2,4-triazol-3-yl)cyclobutyl)phenyl)imidazo[1,2-a]pyridine-8-carboxamide

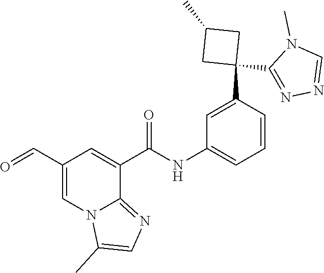 [SMILES: Cc1cnc2c(C(=O)Nc3cccc([C@]4(c5nncn5C)C[C@@H](C)C4)c3)cc(C=O)cn12]

To a solution of 6-formyl-3-methylimidazo[1,2-a]pyridine-8-carboxylic acid (279 mg, 1.37 mmol) in DMF (3 mL) was added HATU (623.47 mg, 1.64 mmol), DIEA (353.19 mg, 2.73 mmol) and 3-((1s, 3s)-3-methyl-1-(4-methyl-4H-1,2,4-triazol-3-yl)cyclobutyl)aniline (298.00 mg, 1.23 mmol). The mixture was stirred at 25° C. for 2 hrs. The reaction mixture was filtered and concentrated under reduced pressure to give a residue, which was purified by Prep-HPLC (column: Waters Xbridge Prep OBD C18 150*40 mm*10 um; mobile phase: [water (NH4HCO₃)-ACN]; B %: 30%-60%, 8 min) to give the product (230 mg, 39.28% yield). MS: M/e 429 (M+1)⁺.

### Step 3: 6-(((3R,4S)-4-fluoro-3-methylpiperidin-1-yl)methyl)-3-methyl-N-(3-((1s,3S)-3-methyl-1-(4-methyl-4H-1,2,4-triazol-3-yl)cyclobutyl)phenyl)imidazo[1,2-a]pyridine-8-carboxamide

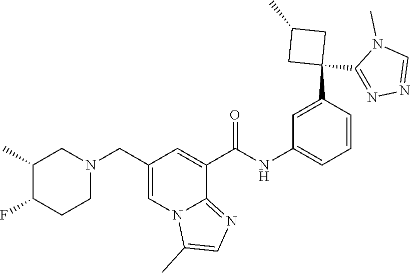 [SMILES: Cc1cnc2c(C(=O)Nc3cccc([C@]4(c5nncn5C)C[C@@H](C)C4)c3)cc(CN3CC[C@H](F)[C@H](C)C3)cn12]

In a mixture of 6-formyl-3-methyl-N-(3-((1s, 3s)-3-methyl-1-(4-methyl-4H-1,2,4-triazol-3-yl)cyclobutyl)phenyl)imidazo[1,2-a]pyridine-8-carboxamide (0.02 g, 46.68 mol, 1 eq) (3R,4S)-4-fluoro-3-methyl-piperidine (14.34 mg, 93.35 mol, 2 eq, HCl), TEA (9.45 mg, 93.35 mol, 12.99 μL, 2 eq), DCE (2 mL) was added NaBH(OAc)₃ (39.57 mg, 186.70 μmol, 4 eq) at 20° C. The mixture was stirred at 20° C. for 12 hrs. The mixture was diluted with DCM (30 ml), adjusted to pH=8 with saturated NaHCO₃ aqueous solution, extracted with DCM (50 mL*2), The combined organic layers were dried over Na₂SO₄, filtered and concentrated under reduced pressure to give a residue. The residue was purified by Prep-HPLC (column: Waters Xbridge BEH C18 100*30 mm*10 um; mobile phase: [water (NH₄HCO₃)-ACN]; B %: 35%-65%, 8 min). Compound 6-(((3R,4S)-4-fluoro-3-methylpiperidin-1-yl)methyl)-3-methyl-N-(3-((1s,3S)-3-methyl-1-(4-methyl-4H-1,2,4-triazol-3-yl)cyclobutyl)phenyl)imidazo[1,2-a]pyridine-8-carboxamide (9.89 mg, 18.67 mol, 40.00% yield) was obtained. MS: M/e 530 (M+1)⁺; ¹H NMR (400 MHz, DMSO-d₆) δ ppm 12.44 (s, 1H), 8.45 (s, 1H), 8.30 (s, 1H), 8.12 (d, J=1.14 Hz, 1H), 7.77 (s, 1H), 7.61-7.66 (m, 1H), 7.59 (s, 1H), 7.41 (t, J=7.88 Hz, 1H), 7.15 (d, J=7.88 Hz, 1H), 4.71 (s, 1H), 3.60 (s, 2H), 3.19 (s, 3H), 2.78-2.90 (m, 2H), 2.52-2.69 (m, 8H), 2.20-2.35 (m, 1H), 2.04 (t, J=11.32 Hz, 1H), 1.65-1.93 (m, 3H), 1.09 (d, J=5.24 Hz, 3H), 0.90 (d, J=6.88 Hz, 3H) ppm.

### Compound A47: N-(3-((1s,3S)-3-(cyanomethyl)-1-(4-methyl-4H-1,2,4-triazol-3-yl)cyclobutyl)phenyl)-6-(((3R,4S)-4-fluoro-3-methylpiperidin-1-yl)methyl)-3-methylimidazo[1,2-a]pyridine-8-carboxamide

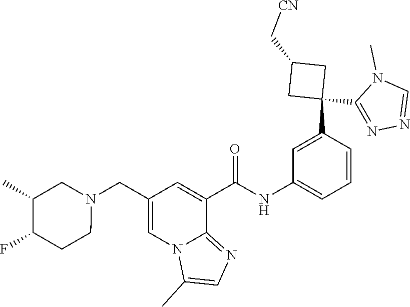 [SMILES: Cc1cnc2c(C(=O)Nc3cccc([C@]4(c5nncn5C)C[C@@H](CC#N)C4)c3)cc(CN3CC[C@H](F)[C@H](C)C3)cn12]

### Step 1: N-(3-((1s, 3s)-3-(cyanomethyl)-1-(4-methyl-4H-1,2,4-triazol-3-yl)cyclobutyl)phenyl)-6-formyl-3-methylimidazo[1,2-a]pyridine-8-carboxamide

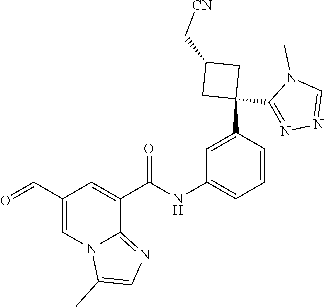 [SMILES: Cc1cnc2c(C(=O)Nc3cccc([C@]4(c5nncn5C)C[C@@H](CC#N)C4)c3)cc(C=O)cn12]

A mixture of 6-formyl-3-methyl-imidazo[1,2-a]pyridine-8-carboxylic acid (0.2 g, 979.52 umol, 1 eq), 2-((1s, 3s)-3-(3-aminophenyl)-3-(4-methyl-4H-1,2,4-triazol-3-yl)cyclobutyl)acetonitrile (237.36 mg, 979.52 umol, 1 eq), HATU (484.18 mg, 1.27 mmol, 1.3 eq), DIEA (253.19 mg, 1.96 mmol, 341.22 uL, 2 eq), DMF (2 mL) was stirred at 30° C. for 2 hrs. The mixture was poured into 60 mL H₂O, extracted with Dichloromethane (60 mL*2), washed with brine(30 mL*2), the combined organic layers were dried over Na₂SO₄, filtered and concentrated under reduced pressure to give a residue. The residue was purified by column chromatography (SiO₂, Petroleum ether/Ethyl acetate=5/1 to 1/1). Compound N-(3-((1s, 3s)-3-(cyanomethyl)-1-(4-methyl-4H-1,2,4-triazol-3-yl)cyclobutyl)phenyl)-6-formyl-3-methylimidazo[1,2-a]pyridine-8-carboxamide (0.08 g, 186.70 umol, 19.06% yield) was obtained.

### Step 2: N-(3-((1s,3S)-3-(cyanomethyl)-1-(4-methyl-4H-1,2,4-triazol-3-yl)cyclobutyl)phenyl)-6-(((3R,4S)-4-fluoro-3-methylpiperidin-1-yl)methyl)-3-methylimidazo[1,2-a]pyridine-8-carboxamide

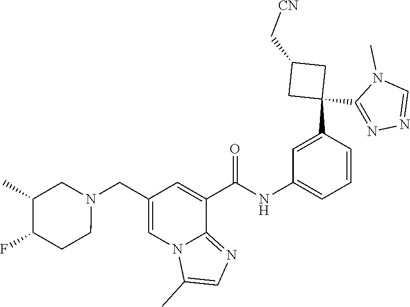 [SMILES: Cc1cnc2c(C(=O)Nc3cccc([C@]4(c5nncn5C)C[C@@H](CC#N)C4)c3)cc(CN3CC[C@H](F)[C@H](C)C3)cn12]

In a mixture of N-(3-((1s, 3s)-3-(cyanomethyl)-1-(4-methyl-4H-1,2,4-triazol-3-yl)cyclobutyl)phenyl)-6-formyl-3-methylimidazo[1,2-a]pyridine-8-carboxamide (0.02 g, 44.10 mol, 1 eq), (3R,4S)-4-fluoro-3-methyl-piperidine (13.55 mg, 88.20 mol, 2 eq, HCl), TEA (8.93 mg, 88.20 μmol, 12.28 L, 2 eq) DCE (1 mL) was added NaBH(OAc)₃ (37.39 mg, 176.41 mol, 4 eq) at 20° C. The mixture was stirred at 20° C. for 12 hrs. The mixture was diluted with DCM (30 ml), adjusted to pH=8 with saturated NaHCO₃ aqueous solution, extracted with DCM (50 ml*2), The combined organic layers were dried over Na₂SO₄, filtered and concentrated under reduced pressure to give a residue. The residue was purified by Prep-HPLC(column: Waters Xbridge BEH C18 100*30 mm*10 um; mobile phase: [water (NH₄HCO₃)-ACN]; B %: 25%-55%, 8 min). Compound N-(3-((1s,3S)-3-(cyanomethyl)-1-(4-methyl-4H-1,2,4-triazol-3-yl)cyclobutyl)phenyl)-6-(((3R,4S)-4-fluoro-3-methylpiperidin-1-yl)methyl)-3-methylimidazo[1,2-a]pyridine-8-carboxamide (10.50 mg, 18.93 mol, 42.92% yield) was obtained. MS: M/e 555 (M+1)⁺; ¹H NMR (400 MHz, DMSO-d₆) δ ppm 12.45 (s, 1H), 8.46 (s, 1H), 8.32 (s, 1H), 8.12 (d, J=1.38 Hz, 1H), 7.76-7.79 (m, 1H), 7.65 (dd, J=1.24 Hz, 1H), 7.59 (d, J=0.88 Hz, 1H), 7.44 (t, J=7.94 Hz, 1H), 7.17-7.25 (m, 1H), 4.52-4.74 (m, 1H), 3.60 (s, 2H), 3.22 (s, 3H), 2.84-2.93 (m, 2H), 2.68-2.81 (m, 5H), 2.54-2.66 (m, 5H), 2.22-2.35 (m, 1H), 2.00-2.11 (m, 1H), 1.65-1.96 (m, 3H), 0.90 (d, J=6.88 Hz, 3H) ppm.

### Compound A48: N-(3-((1r,3R)-3-cyano-1-((4-methyl-4H-1,2,4-triazol-3-yl)methyl)cyclobutyl)phenyl)-6-(((3R,4S)-4-fluoro-3-methylpiperidin-1-yl)methyl)imidazo[1,2-a]pyridine-8-carboxamide

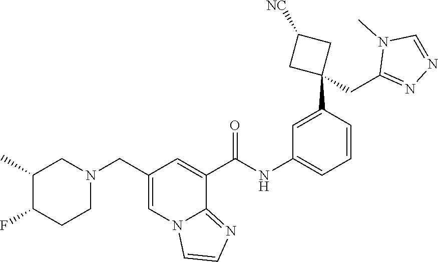 [SMILES: C[C@@H]1CN(Cc2cc(C(=O)Nc3cccc([C@]4(Cc5nncn5C)C[C@@H](C#N)C4)c3)c3nccn3c2)CC[C@@H]1F]

The compound A48 was synthesized starting from the corresponding starting materials according the similar procedures described as those of Compound A5 to get the product (11 mg, 31%). ¹H NMR (400 MHz, CD₃OD) δ 8.64 (s, 1H), 8.23 (s, 1H), 8.16 (s, 1H), 7.97 (s, 1H), 7.74 (s, 1H), 7.69 (d, J=8.0 Hz, 1H), 7.48 (s, 1H), 7.34 (t, J=8.0 Hz, 1H), 6.81 (d, J=8.0 Hz, 1H), 4.70-4.57 (m, 1H), 3.71 (s, 2H), 3.42 (s, 2H), 3.21-3.17 (m, 1H), 3.00 (d, J=8.0 Hz, 4H), 2.81 (s, 3H), 2.77-2.70 (m, 2H), 2.46 (t, J=12.0 Hz, 1H), 2.24 (t, J=12.0 Hz, 1H), 2.02-1.81 (m, 3H), 0.98 (d, J=4.0 Hz, 3H) ppm. MS: M/e 541 (M+1)+

### Compound A49: 6-((((S)-sec-butyl)amino)methyl)-N-(5-((1s,3R)-3-methyl-1-(4-methyl-4H-1,2,4-triazol-3-yl)cyclobutyl)pyridin-3-yl)imidazo[1,2-a]pyridine-8-carboxamide

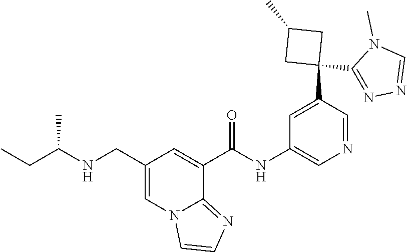 [SMILES: CC[C@H](C)NCc1cc(C(=O)Nc2cncc([C@]3(c4nncn4C)C[C@@H](C)C3)c2)c2nccn2c1]

### Step 1: methyl (S)-6-((sec-butylamino)methyl)imidazo[1,2-a]pyridine-8-carboxylate

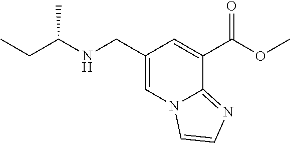 [SMILES: CC[C@H](C)NCc1cc(C(=O)OC)c2nccn2c1]

A mixture of methyl 6-formylimidazo[1,2-a]pyridine-8-carboxylate (0.2 g, 489.76 μmol), (2S)-butan-2-amine (107.35 mg, 979.52 mol), CH₃COOH (44.12 mg, 734.64 μmol), NaBH₃CN (92.33 mg, 1.47 mmol) and TEA (99.12 mg, 979.52 μmol) in MeOH (2 mL) was degassed and purged with N₂ for 3 times, and then the mixture was stirred at 20° C. for 16 hr under N₂ atmosphere. The reaction mixture was quenched by addition H₂O 10 mL at 20° C., and then extracted with EtOAc 30 mL (10 mL*3). The combined organic layers were washed with brine 30 mL (10 mL*3), dried over Na2SO4, filtered and concentrated under reduced pressure to give a residue. The resulting residue was purified by prep-TLC (SiO2, DCM:MeOH=3:1) to give the product (0.06 g, 46.88% yield). MS: M/e 262 (M+1)⁺; ¹H NMR (400 MHz, DMSO-d₆) δ=8.71 (s, 1H), 8.10-7.96 (m, 1H), 7.90 (s, 1H), 7.61 (s, 1H), 3.89 (s, 3H), 3.83-3.65 (m, 2H), 1.47 (d, J=4.32 Hz, 1H), 1.36-1.17 (m, 2H), 1.06-0.96 (m, 3H), 0.84 (t, J=7.52 Hz, 3H) ppm.

### Step 2: (S)-6-((sec-butylamino)methyl)imidazo[1,2-a]pyridine-8-carboxylic acid

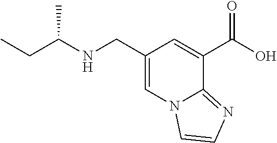 [SMILES: CC[C@H](C)NCc1cc(C(=O)O)c2nccn2c1]

A mixture of methyl (S)-6-((sec-butylamino)methyl)imidazo[1,2-a]pyridine-8-carboxylate (0.06 g, 229.60 mol, 1 eq), LiOH·H2O (9.64 mg, 229.60 mol, 1 eq) in THF (1 mL) and MeOH (1 mL) and H₂O (1 mL) was degassed and purged with N₂ for 3 times, and then the mixture was stirred at 25° C. for 1 hr under N2 atmosphere. The reaction mixture was concentrated under reduced pressure to give a residue. The crude product was used into the next step without further purification to give the product (47 mg, 80.84% yield). MS: M/e 248 (M+1)⁺; ¹H NMR (400 MHz, DMSO-d₆) δ=8.48 (s, 1H), 7.94 (d, J=1.0 Hz, 1H), 7.87 (s, 1H), 7.48 (s, 1H), 3.78-3.63 (m, 2H), 1.97-1.90 (m, 1H), 1.55-1.40 (m, 1H), 1.34-1.22 (m, 1H), 1.00 (d, J=6.32 Hz, 3H), 0.85 (t, J=7.4 Hz, 3H) ppm.

### Step 3: 6-((((S)-sec-butyl)amino)methyl)-N-(5-((1s,3R)-3-methyl-1-(4-methyl-4H-1,2,4-triazol-3-yl)cyclobutyl)pyridin-3-yl)imidazo[1,2-a]pyridine-8-carboxamide

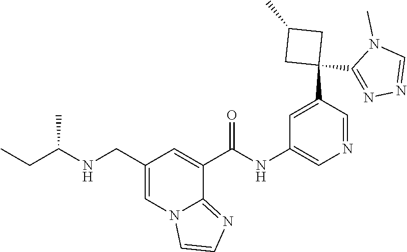 [SMILES: CC[C@H](C)NCc1cc(C(=O)Nc2cncc([C@]3(c4nncn4C)C[C@@H](C)C3)c2)c2nccn2c1]

To a stirred solution of (S)-6-((sec-butylamino)methyl)imidazo[1,2-a]pyridine-8-carboxylic acid (47 mg, 148.48 mol) and 5-[3-methyl-1-(4-methyl-1,2,4-triazol-3-yl)cyclobutyl]pyridin-3-amine (36.13 mg, 148.48 μmol) in PYRIDINE (1 mL) was added T3P (283.47 mg, 445.45 μmol) at 20° C. The resulting mixture was stirred for 3 h at 20° C. The reaction mixture was quenched by addition H₂O 10 mL at 20° C., and then extracted with EtOAc 30 mL (10 mL*3). The combined organic layers were washed with brine 30 mL (10 mL*3), dried over Na₂SO₄, filtered and concentrated under reduced pressure to give a residue. The residue was purified by prep-HPLC (column: Waters Xbridge BEH C18 100*30 mm*10 um; mobile phase: [H2O (10 mM NH4HCO₃)-ACN]; gradient: 20%-50% B over 8.0 min) to give product (11.19 mg, 23.68 mol, 15.95% yield). MS: M/e 472 (M+1)⁺; ¹H NMR (400 MHz, DMSO-d₆) δ=12.63-12.36 (m, 1H), 8.86 (d, J=2.32 Hz, 1H), 8.76 (s, 1H), 8.41 (d, J=1.92 Hz, 1H), 8.34 (s, 1H), 8.19-8.12 (m, 3H), 7.81-7.78 (m, 1H), 3.85-3.73 (m, 2H), 3.24 (s, 3H), 2.94-2.88 (m, 2H), 2.62-2.56 (m, 4H), 1.53-1.41 (m, 1H), 1.36-1.20 (m, 1H), 1.12-1.06 (m, 3H), 1.01 (d, J=6.32 Hz, 3H), 0.88-0.82 (m, 3H) ppm.

### Compound A50: 6-({[(1S)-1-cyclobutylethyl]amino}methyl)-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide

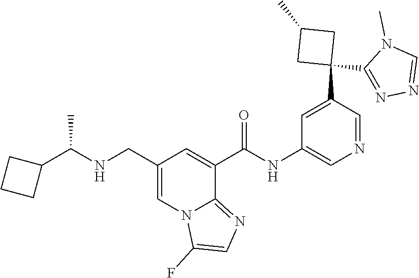 [SMILES: C[C@H](NCc1cc(C(=O)Nc2cncc([C@]3(c4nncn4C)C[C@@H](C)C3)c2)c2ncc(F)n2c1)C1CCC1]

### Step 1: methyl 6-({[(1S)-1-cyclobutylethyl]amino}methyl)-3-fluoroimidazo[1,2-a]pyridine-8-carboxylate

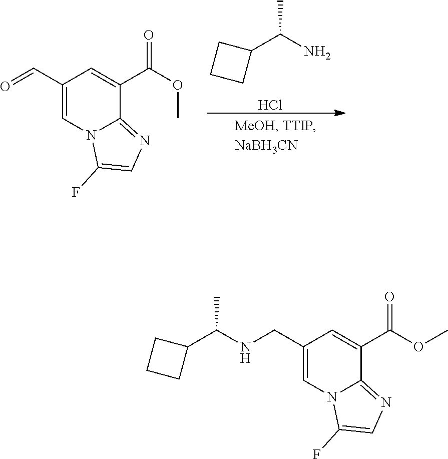

To a stirred mixture of methyl 3-fluoro-6-formylimidazo[1,2-a]pyridine-8-carboxylate (150 mg, 0.596 mmol, 1 equiv, 88.3%) and (1S)-1-cyclobutylethanamine hydrochloride (128 mg, 0.896 mmol, 1.50 equiv, 95%) in MeOH (5 mL, 99%) were added tetrakis(propan-2-yloxy)titanium (714 mg, 2.387 mmol, 4.00 equiv, 95%) dropwise at room temperature. The resulting mixture was stirred for 3 hrs at room temperature. To the above mixture was added NaBH₃CN (79 mg, 1.194 mmol, 2.00 equiv, 95%) in portions at room temperature. The resulting mixture was stirred for additional 3 hrs at room temperature. The resulting mixture was concentrated under vacuum. The residue was purified by silica gel column chromatography, eluted with CH₂Cl₂/MeOH (7:1) to afford methyl 6-({[(1S)-1-cyclobutylethyl]amino}methyl)-3-fluoroimidazo[1,2-a]pyridine-8-carboxylate (110 mg, 50.64%). MS: M/e 306 (M+1)⁺.

### Step 2: 6-({[(1S)-1-cyclobutylethyl]amino}methyl)-3-fluoroimidazo[1,2-a]pyridine-8-carboxylic acid

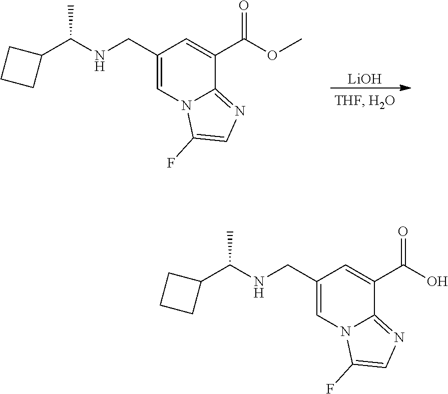 [SMILES: COC(=O)c1cc(CN[C@@H](C)C2CCC2)cn2c(F)cnc12 ; "LiOH" ; C[C@H](NCc1cc(C(=O)O)c2ncc(F)n2c1)C1CCC1]

To a stirred solution of methyl 6-({[(1S)-1-cyclobutylethyl]amino}methyl)-3-fluoroimidazo[1,2-a]pyridine-8-carboxylate (110 mg, 0.302 mmol, 1 equiv, 83.8%) in THF (2 mL, 99%) and H₂O (1 mL, 99%) were added LiOH (16 mg, 0.635 mmol, 2.10 equiv, 95%) in portions at room temperature. The resulting mixture was stirred for 2 hrs at room temperature. The mixture was acidified to pH=4 with HCl (1 M). The resulting mixture was concentrated under vacuum. The residue was purified by reversed-phase flash chromatography with the following conditions: column, C18 silica gel; mobile phase, MeCN in Water (0.1% FA), 0%; detector, UV 254 nm. This resulted in 6-({[(1S)-1-cyclobutylethyl]amino}methyl)-3-fluoroimidazo[1,2-a]pyridine-8-carboxylic acid (33 mg, 31.74%). MS: M/e 292 (M+1)⁺.

### Step 3: 6-({[(1S)-1-cyclobutylethyl]amino}methyl)-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide

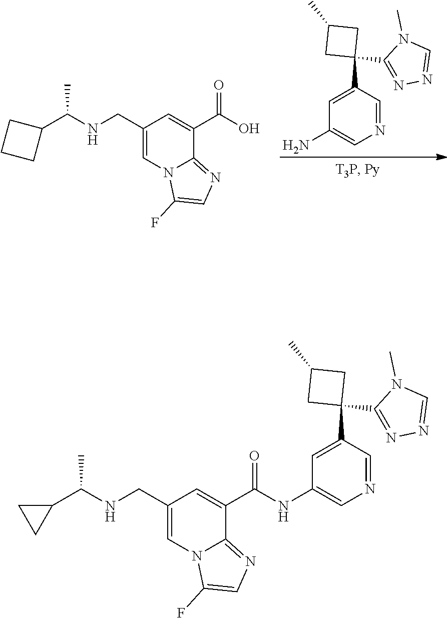 [SMILES: C[C@H](NCc1cc(C(=O)O)c2ncc(F)n2c1)C1CCC1 ; Cn1cnnc1[C@]1(c2cncc(N)c2)C[C@H](C)C1 ; C[C@H](NCc1cc(C(=O)Nc2cncc([C@]3(c4nncn4C)C[C@@H](C)C3)c2)c2ncc(F)n2c1)C1CC1]

To a stirred mixture of 6-({[(1S)-1-cyclobutylethyl]amino}methyl)-3-fluoroimidazo[1,2-a]pyridine-8-carboxylic acid (30 mg, 0.087 mmol, 1 equiv, 84.6%) and 5-[(1r, 3s)-3-methyl-1-(4-methyl-1,2,4-triazol-3-yl)cyclobutyl]pyridin-3-amine (23 mg, 0.090 mmol, 1.03 equiv, 95.0%) in Pyridine (1.5 mL, 95%) was added T₃P (555 mg, 0.872 mmol, 10.01 equiv, 50%) dropwise at room temperature. The resulting mixture was stirred for 30 min at room temperature. The resulting mixture was concentrated under vacuum. The crude product (100 mg) was purified by Prep-HPLC with the following conditions (2 #SHIMADZU (HPLC-01)): Column, YMC-Actus Triart C18 ExRS, 30*150 mm, 5 μm; mobile phase, 10 mmol NH₄HCO₃+0.05% NH₃H₂O and ACN (12% ACN up to 42% in 8 min); This resulted in 6-({[(1S)-1-cyclobutylethyl]amino}methyl)-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide (9.8 mg, 21.43%). MS: M/e 517 (M+1); ¹H NMR (300 MHz, DMSO-d₆) δ 12.02 (s, 1H), 8.85 (d, J=2.3 Hz, 1H), 8.49 (s, 1H), 8.41 (d, J=2.1 Hz, 1H), 8.34 (s, 1H), 8.18 (d, J=1.5 Hz, 1H), 8.14-8.07 (m, 1H), 7.62 (d, J=6.9 Hz, 1H), 3.91-3.72 (m, 2H), 3.24 (s, 3H), 2.91 (d, J=3.6 Hz, 2H), 2.59 (d, J=6.8 Hz, 3H), 2.51-2.40 (m, 1H), 2.23-2.12 (m, 1H), 2.06-1.94 (m, 1H), 1.93-1.82 (m, 1H), 1.82-1.56 (m, 4H), 1.10 (d, J=4.8 Hz, 3H), 0.91 (d, J=6.1 Hz, 3H) ppm.

### Compound A51: 6-(((cyclopentylmethyl)amino)methyl)-3-fluoro-N-(3-((1s, 3s)-3-methyl-1-(4-methyl-4H-1,2,4-triazol-3-yl)cyclobutyl)phenyl)imidazo[1,2-a]pyridine-8-carboxamide

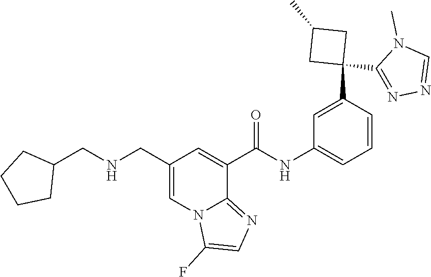 [SMILES: Cn1cnnc1[C@]1(c2cccc(NC(=O)c3cc(CNCC4CCCC4)cn4c(F)cnc34)c2)C[C@H](C)C1]

The compound A51 was synthesized starting from the corresponding starting materials according the similar procedures described as those of Compound A19 (12.97 mg, 25.15 mol, 11.39% yield) was obtained. MS: M/e 516 (M+1)⁺; ¹H NMR (400 MHz, DMSO-d₆) δ ppm 11.94 (s, 1H), 8.45 (s, 1H), 8.30 (s, 1H), 8.16 (s, 1H), 7.71 (s, 1H), 7.66 (d, J=8.00 Hz, 1H), 7.61 (d, J=6.88 Hz, 1H), 7.42 (t, J=7.94 Hz, 1H), 7.16 (d, J=7.74 Hz, 1H), 3.80 (s, 2H), 3.19 (s, 3H), 2.78-2.89 (m, 2H), 2.55 (q, J=6.74 Hz, 3H), 2.42 (d, J=7.12 Hz, 2H), 1.98 (d, J=14.88 Hz, 1H), 1.62-1.76 (m, 2H), 1.39-1.56 (m, 4H), 1.13-1.24 (m, 2H), 1.09 (d, J=5.12 Hz, 3H) ppm.

### Compound A52: 6-(((cyclobutylmethyl)amino)methyl)-3-fluoro-N-(3-((1s, 3s)-3-methyl-1-(4-methyl-4H-1,2,4-triazol-3-yl)cyclobutyl)phenyl)imidazo[1,2-a]pyridine-8-carboxamide

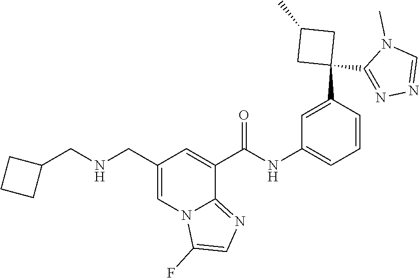 [SMILES: Cn1cnnc1[C@]1(c2cccc(NC(=O)c3cc(CNCC4CCC4)cn4c(F)cnc34)c2)C[C@H](C)C1]

The compound A52 was synthesized starting from the corresponding starting materials according the similar procedures described as those of Compound A19 to give the title product (16.94 mg, 33.77 umol, 15.30% yield). MS: M/e 502 (M+1)⁺; H NMR (400 MHz, DMSO-d₆) δ ppm 11.94 (s, 1H), 8.46 (s, 1H), 8.29 (s, 1H), 8.16 (d, J=1.00 Hz, 1H), 7.71 (s, 1H), 7.66 (d, J=8.00 Hz, 1H), 7.62 (d, J=6.88 Hz, 1H), 7.42 (t, J=7.88 Hz, 1H), 7.16 (d, J=7.63 Hz, 1H), 3.79 (s, 2H), 3.19 (s, 3H), 2.84 (d, J=3.25 Hz, 2H), 2.48-2.59 (m, 6H), 2.41 (dt, J=14.82, 7.47 Hz, 1H), 1.93-2.05 (m, 2H), 1.74-1.89 (m, 2H), 1.56-1.69 (m, 2H), 1.09 (d, J=5.00 Hz, 3H) ppm.

### Compound A53: 6-(((cyclobutylmethyl)amino)methyl)-3-methyl-N-(3-((1s, 3s)-3-methyl-1-(4-methyl-4H-1,2,4-triazol-3-yl)cyclobutyl)phenyl)imidazo[1,2-a]pyridine-8-carboxamide

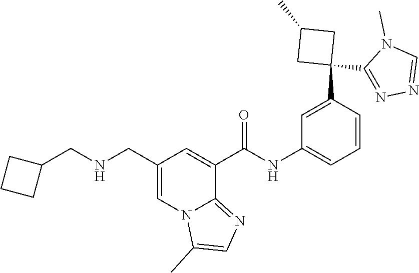 [SMILES: Cc1cnc2c(C(=O)Nc3cccc([C@]4(c5nncn5C)C[C@@H](C)C4)c3)cc(CNCC3CCC3)cn12]

The compound A53 was synthesized starting from the corresponding starting materials according the similar procedures described as those of Compound A46 to give the product (85.32 mg, 48.98% yield). MS: M/e 498 (M+1)⁺; H NMR (400 MHz, DMSO-d₆) δ=12.45 (s, 1H), 8.43 (s, 1H), 8.30 (s, 1H), 8.15 (s, 1H), 7.75 (s, 1H), 7.65 (br d, J=8.0 Hz, 1H), 7.57 (s, 1H), 7.41 (t, J=8.0 Hz, 1H), 7.15 (d, J=8.0 Hz, 1H), 3.79 (s, 2H), 3.20 (s, 3H), 2.84 (br d, J=4.0 Hz, 2H), 2.65-2.51 (m, 7H), 2.48-2.22 (m, 2H), 2.04-1.93 (m, 2H), 1.88-1.72 (m, 2H), 1.70-1.57 (m, 2H), 1.09 (br d, J=4.0 Hz, 3H) ppm.

### Compound A54:6-(((3R,4S)-4-fluoro-3-methylpiperidin-1-yl)methyl)-N-(5-((1s,3S)-3-methyl-1-(4-methyl-4H-1,2,4-triazol-3-yl)cyclobutyl)pyridin-3-yl)imidazo[1,2-a]pyridine-8-carboxamide

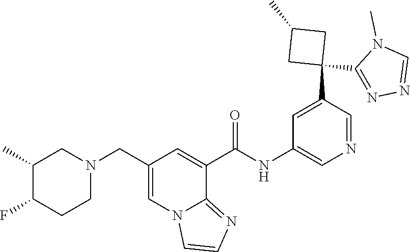 [SMILES: C[C@@H]1CN(Cc2cc(C(=O)Nc3cncc([C@]4(c5nncn5C)C[C@@H](C)C4)c3)c3nccn3c2)CC[C@@H]1F]

### Step 1: 6-formyl-N-(5-((1s, 3s)-3-methyl-1-(4-methyl-4H-1,2,4-triazol-3-yl)cyclobutyl)pyridin-3-yl)imidazo[1,2-a]pyridine-8-carboxamide

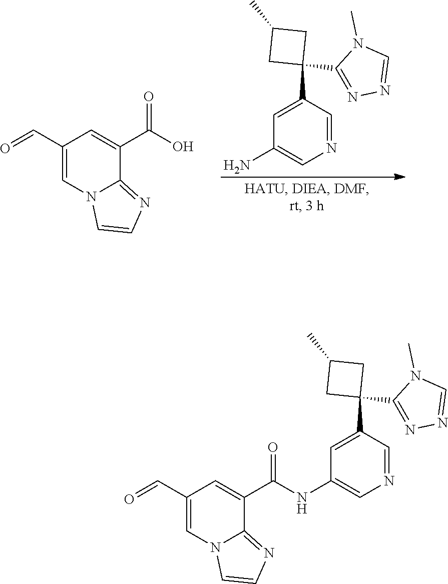 [SMILES: O=Cc1cc(C(=O)O)c2nccn2c1 ; Cn1cnnc1[C@]1(c2cncc(N)c2)C[C@H](C)C1 ; Cn1cnnc1[C@]1(c2cncc(NC(=O)c3cc(C=O)cn4ccnc34)c2)C[C@H](C)C1]

To a stirred solution of 6-formylimidazo[1,2-a]pyridine-8-carboxylic acid (40 mg, 0.200 mmol, 1 equiv, 95%) and DIEA (108.75 mg, 0.800 mmol, 4 equiv, 95%) in DMF (3 ml) were added HATU (119.97 mg, 0.300 mmol, 1.5 equiv, 95%) and 5-[(1r, 3s)-3-methyl-1-(4-methyl-1,2,4-triazol-3-yl)cyclobutyl]pyridin-3-amine (51.18 mg, 0.200 mmol, 1 equiv, 95%) at room temperature under nitrogen atmosphere. The resulting mixture was stirred for additional 3 hrs at room temperature. The resulting mixture was concentrated under vacuum. The residue was purified by reversed-phase flash chromatography with the following conditions: column, C18 silica gel; mobile phase, MeCN in Water (10 mmol/L NH₄HCO₃), 0% to 100% gradient in 30 min; detector, UV 254 nm. This resulted in 6-formyl-N-{5-[(1r, 3s)-3-methyl-1-(4-methyl-1,2,4-triazol-3-yl)cyclobutyl]pyridin-3-yl}imidazo[1,2-a]pyridine-8-carboxamide (71 mg, 85.52%). MS: M/e 416 (M+1)⁺.

### Step 2: 6-(((3R,4S)-4-fluoro-3-methylpiperidin-1-yl)methyl)-N-(5-((1s,3S)-3-methyl-1-(4-methyl-4H-1,2,4-triazol-3-yl)cyclobutyl)pyridin-3-yl)imidazo[1,2-a]pyridine-8-carboxamide

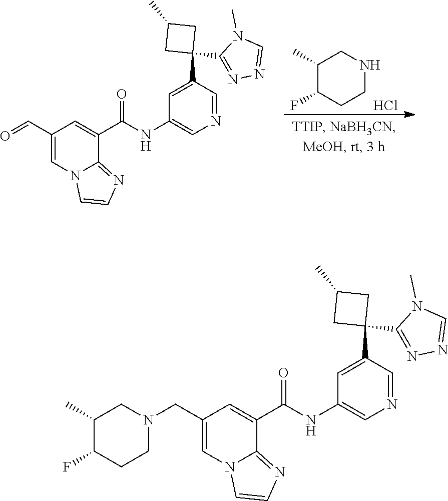

To a stirred solution of 6-formyl-N-{5-[(1r, 3s)-3-methyl-1-(4-methyl-1,2,4-triazol-3-yl)cyclobutyl]pyridin-3-yl}imidazo[1,2-a]pyridine-8-carboxamide (25 mg, 0.057 mmol, 1 equiv, 95%) and (3R,4S)-4-fluoro-3-methylpiperidine hydrochloride (18.49 mg, 0.114 mmol, 2 equiv, 95%) in MeOH (2 mL) was added tetrakis(propan-2-yloxy)titanium (68.41 mg, 0.228 mmol, 4 equiv, 95%) at room temperature under nitrogen atmosphere. The resulting mixture was stirred for additional 1 h at room temperature. To the above mixture was added NaBH₃CN (7.56 mg, 0.114 mmol, 2 equiv, 95%). The resulting mixture was stirred for additional 1 h at room temperature. The crude product was purified by Chiral-Prep-HPLC with the following conditions (2 #SHIMADZU (HPLC-01)): Column, XBridge Prep OBD C18 Column, 30*150 mm, 5 μm; mobile phase, 10 mmol NH₄HCO₃+0.05% NH₃H₂O and ACN (25% ACN up to 55% in 8 min); This resulted in 6-{[(3R,4S)-4-fluoro-3-methylpiperidin-1-yl]methyl}-N-{5-[(1r, 3s)-3-methyl-1-(4-methyl-1,2,4-triazol-3-yl)cyclobutyl]pyridin-3-yl}imidazo[1,2-a]pyridine-8-carboxamide (9.0 mg, 29.62%). MS: M/e 517 (M+1)⁺. ¹H NMR (300 MHz, DMSO-d₆) δ 12.49 (s, 1H), 8.81 (m, 2H), 8.45-8.33 (m, 2H), 8.18-8.08 (m, 3H), 7.81 (m, 1H), 4.65 (m, 1H), 3.65-3.49 (m, 2H), 3.24 (s, 3H), 2.92 (s, 2H), 2.59 (m, 5H), 2.25 (m, 1H), 2.03 (m, 1H), 1.93-1.66 (m, 3H), 1.10 (m, 3H), 0.90 (m, 3H) ppm.

### compound A55: 6-{[(3R,4S)-4-fluoro-3-methylpiperidin-1-yl]methyl}-3-methyl-N-{5-[(1r, 3s)-3-methyl-1-(4-methyl-1,2,4-triazol-3-yl)cyclobutyl]pyridin-3-yl}imidazo[1,2-a]pyridine-8-carboxamide

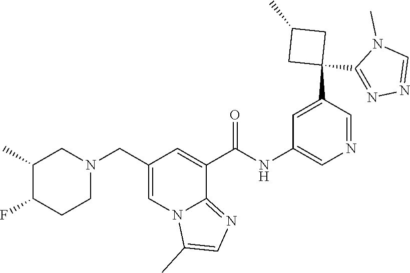 [SMILES: Cc1cnc2c(C(=O)Nc3cncc([C@]4(c5nncn5C)C[C@@H](C)C4)c3)cc(CN3CC[C@H](F)[C@H](C)C3)cn12]

### Step 1: 6-formyl-3-methyl-N-{5-[(1r, 3s)-3-methyl-1-(4-methyl-1,2,4-triazol-3-yl)cyclobutyl]pyridin-3-yl}imidazo[1,2-a]pyridine-8-carboxamide

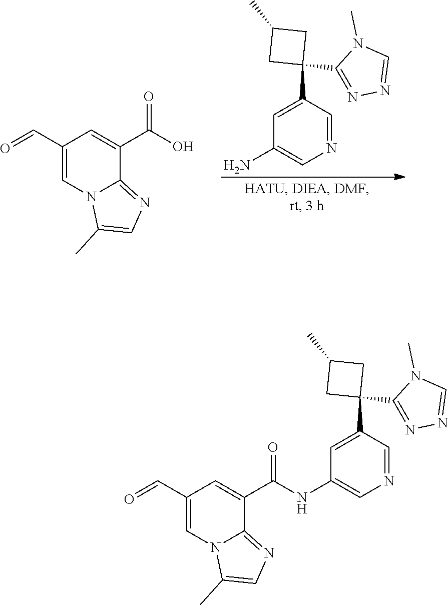 [SMILES: Cc1cnc2c(C(=O)O)cc(C=O)cn12 ; Cn1cnnc1[C@]1(c2cncc(N)c2)C[C@H](C)C1 ; Cc1cnc2c(C(=O)Nc3cncc([C@]4(c5nncn5C)C[C@@H](C)C4)c3)cc(C=O)cn12]

To a stirred solution of 6-formyl-3-methylimidazo[1,2-a]pyridine-8-carboxylic acid (45 mg, 0.198 mmol, 1 equiv, 89.9%) in DMF (3 mL, 100%) was added HATU (118.95 mg, 0.297 mmol, 1.5 equiv, 95%) and DIEA (8.99 mg, 0.066 mmol, 3 equiv, 95%) at room temperature. The resulting mixture was stirred for 10 min at room temperature. To the above mixture was added 5-[(1r, 3s)-3-methyl-1-(4-methyl-1,2,4-triazol-3-yl)cyclobutyl]pyridin-3-amine (50.74 mg, 0.198 mmol, 1 equiv, 95%) at room temperature. The resulting mixture was stirred for additional 2 hrs at room temperature. The resulting mixture was concentrated under vacuum. The residue was purified by reversed-phase flash chromatography with the following conditions: column, C18 silica gel; mobile phase, MeCN in Water (0.1% NH₃·H₂O), 20% to 50% gradient in 10 min; detector, UV 254 nm. This resulted in 6-formyl-3-methyl-N-{5-[(1r, 3s)-3-methyl-1-(4-methyl-1,2,4-triazol-3-yl)cyclobutyl]pyridin-3-yl}imidazo[1,2-a]pyridine-8-carboxamide (30 mg, 33.49%). MS: M/e 430 (M+1)⁺.

### Step 2: 6-{[(3R,4S)-4-fluoro-3-methylpiperidin-1-yl]methyl}-3-methyl-N-{5-[(1r, 3s)-3-methyl-1-(4-methyl-1,2,4-triazol-3-yl)cyclobutyl]pyridin-3-yl}imidazo[1,2-a]pyridine-8-carboxamide

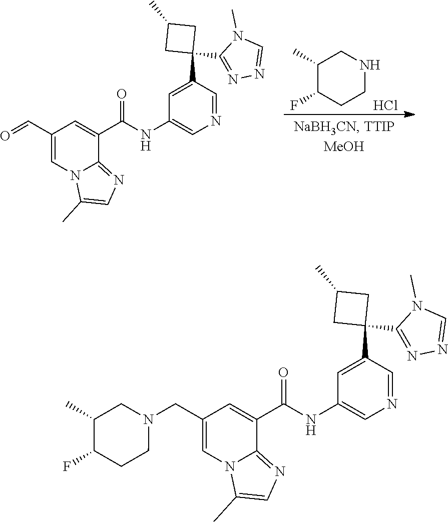

To a stirred solution of 6-formyl-3-methyl-N-{5-[(1r, 3s)-3-methyl-1-(4-methyl-1,2,4-triazol-3-yl)cyclobutyl]pyridin-3-yl}imidazo[1,2-a]pyridine-8-carboxamide (10 mg, 0.022 mmol, 1 equiv, 95%) and (3R,4S)-4-fluoro-3-methylpiperidine hydrochloride (7.15 mg, 0.044 mmol, 2 equiv, 95%) in MeOH (2 mL, 100%) were added tetrakis(propan-2-yloxy)titanium (26.47 mg, 0.088 mmol, 4 equiv, 95%) at room temperature. The resulting mixture was stirred for 1 h at room temperature. To the above mixture was added NaBH₃CN (2.93 mg, 0.044 mmol, 2 equiv, 95%) at room temperature. The resulting mixture was stirred for additional 16 hrs at room temperature. The resulting mixture was concentrated under reduced pressure. The crude product was purified by Chiral-Prep-HPLC with the following conditions (2 #SHIMADZU (HPLC-01)): Column, XBridge Prep OBD C18 Column, 30*150 mm, 5 μm; mobile phase, 10 mmol NH₄HCO₃+0.05% NH₃H₂O and ACN (30% ACN up to 60% in 8 min); This resulted in 6-{[(3R,4S)-4-fluoro-3-methylpiperidin-1-yl]methyl}-3-methyl-N-{5-[(1r, 3s)-3-methyl-1-(4-methyl-1,2,4-triazol-3-yl)cyclobutyl]pyridin-3-yl}imidazo[1,2-a]pyridine-8-carboxamide (2 mg, 16.58%). MS: M/e 531 (M+1)⁺; ¹H NMR (400 MHz, DMSO-d₆) δ 12.55 (s, 1H), 8.84 (d, J=2.3 Hz, 1H), 8.49 (d, J=1.6 Hz, 1H), 8.41 (d, J=2.1 Hz, 1H), 8.35 (s, 1H), 8.18-8.11 (m, 2H), 7.60 (d, J=1.2 Hz, 1H), 4.65 (d, J=50.1 Hz, 1H), 3.61 (s, 2H), 3.24 (s, 3H), 2.91 (d, J=3.8 Hz, 2H), 2.62-2.52 (m, 8H), 2.32-2.21 (m, 1H), 2.10-1.99 (m, 1H), 1.92-1.80 (m, 3H), 1.10 (d, J=4.7 Hz, 3H), 0.90 (d, J=6.8 Hz, 3H) ppm.

### Compound A56: 6-{[(cyclobutylmethyl)amino]methyl}-3-methyl-N-{5-[(1r, 3s)-3-methyl-1-(4-methyl-1,2,4-triazol-3-yl)cyclobutyl]pyridin-3-yl}imidazo[1,2-a]pyridine-8-carboxamide

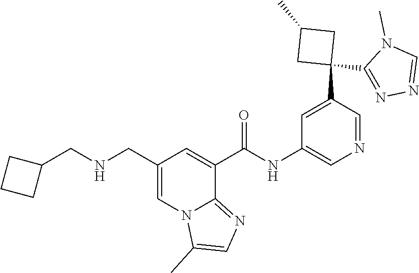 [SMILES: Cc1cnc2c(C(=O)Nc3cncc([C@]4(c5nncn5C)C[C@@H](C)C4)c3)cc(CNCC3CCC3)cn12]

The compound A56 was synthesized starting from the corresponding starting materials according the similar procedures described as those of Compound A55 to give the product (12.3 mg, 35.54%). MS: M/e 499 (M+1)⁺; ¹H NMR (300 MHz, DMSO-d₆) δ 12.56 (s, 1H), 8.84 (d, J=2.3 Hz, 1H), 8.47 (d, J=1.6 Hz, 1H), 8.40 (d, J=2.1 Hz, 1H), 8.34 (s, 1H), 8.20-8.10 (m, 2H), 7.59 (d, J=1.1 Hz, 1H), 3.80 (s, 2H), 3.24 (s, 3H), 2.91 (d, J=3.9 Hz, 2H), 2.64-2.55 (m, 4H), 2.53 (d, J=1.2 Hz, 4H), 2.49-2.33 (m, 2H), 2.07-1.92 (m, 2H), 1.91-1.75 (m, 2H), 1.75-1.54 (m, 2H), 1.10 (d, J=4.9 Hz, 3H) ppm.

### Compound A57: 3-chloro-6-(((cyclobutylmethyl)amino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

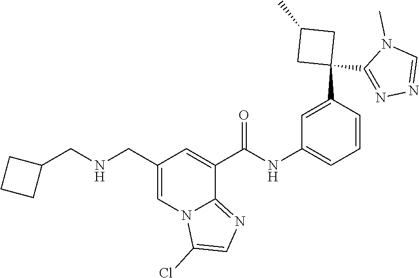 [SMILES: Cn1cnnc1[C@]1(c2cccc(NC(=O)c3cc(CNCC4CCC4)cn4c(Cl)cnc34)c2)C[C@H](C)C1]

### Step 1: 3-chloro-6-formyl-N-(3-((1s, 3s)-3-methyl-1-(4-methyl-4H-1,2,4-triazol-3-yl)cyclobutyl)phenyl)imidazo[1,2-a]pyridine-8-carboxamide

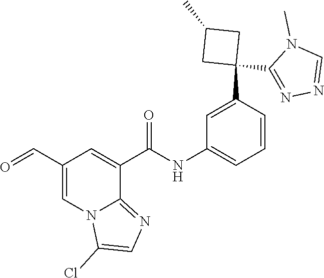 [SMILES: Cn1cnnc1[C@]1(c2cccc(NC(=O)c3cc(C=O)cn4c(Cl)cnc34)c2)C[C@H](C)C1]

A mixture of 3-chloro-6-formyl-imidazo[1,2-a]pyridine-8-carboxylic acid (72.10 mg, 320.99 umol), 3-((1s, 3s)-3-methyl-1-(4-methyl-4H-1,2,4-triazol-3-yl)cyclobutyl)aniline (77.78 mg, 320.99 umol), HATU (146.46 mg, 385.19 umol), DIEA (124.46 mg, 962.98 umol, 167.73 uL), DMF (1 mL) was stirred at 25° C. for 2 hrs. The mixture was poured into 20 ml H₂O, the solid was filtered, the solid was concentrated in vacuo. Compound 3-chloro-6-formyl-N-(3-((1s, 3s)-3-methyl-1-(4-methyl-4H-1,2,4-triazol-3-yl)cyclobutyl)phenyl)imidazo[1,2-a]pyridine-8-carboxamide (0.08 g, 178.21 umol, 55.52% yield) was obtained. MS: M/e 449 (M+1)⁺.

### Step 2: 3-chloro-6-(((cyclobutylmethyl)amino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

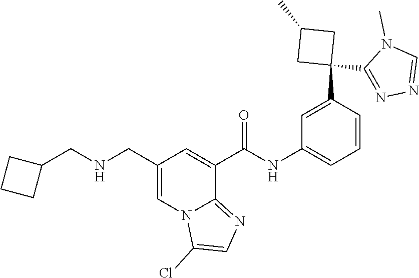 [SMILES: Cn1cnnc1[C@]1(c2cccc(NC(=O)c3cc(CNCC4CCC4)cn4c(Cl)cnc34)c2)C[C@H](C)C1]

A mixture of 3-chloro-6-formyl-N-(3-((1s, 3s)-3-methyl-1-(4-methyl-4H-1,2,4-triazol-3-yl)cyclobutyl)phenyl)imidazo[1,2-a]pyridine-8-carboxamide (0.04 g, 89.11 umol, 1 eq), cyclobutylmethanamine (43.34 mg, 356.42 umol, HCl), TEA (36.07 mg, 356.42 umol, 49.61 uL), NaBH(OAc)₃ (56.66 mg, 267.32 umol), DCE (2 mL) was stirred at 20° C. for 12 hrs. The residue was purified by Prep-HPLC(column: Waters Xbridge Prep OBD C18 150*40 mm*10 um; mobile phase: [water (NH₄HCO₃)-ACN]; B %: 45%-75%, 8 min). Compound 3-chloro-6-(((cyclobutylmethyl)amino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide (11.37 mg, 21.95 umol, 24.63% yield) was obtained. MS: M/e 518 (M+1)⁺. ¹H NMR (400 MHz, DMSO-d₆) δ 11.96 (s, 1H), 8.51 (s, 1H), 8.30 (s, 1H), 8.22 (s, 1H), 7.95 (s, 1H), 7.72 (s, 1H), 7.66 (d, J=8.00 Hz, 1H), 7.42 (t, J=7.94 Hz, 1H), 7.17 (d, J=7.88 Hz, 1H), 3.83 (s, 2H), 3.19 (s, 3H), 2.83 (s, 2H), 2.54 (d, J=7.00 Hz, 5H), 2.41 (d, J=14.88 Hz, 2H), 1.93-2.05 (m, 2H), 1.74-1.88 (m, 2H), 1.57-1.69 (m, 2H), 1.09 (d, J=4.88 Hz, 3H) ppm.

### Compound A58: 3-chloro-6-((((S)-1-cyclopropylethyl)amino)methyl)-N-(3-((1s,3R)-3-methyl-1-(4-methyl-4H-1,2,4-triazol-3-yl)cyclobutyl)phenyl)imidazo[1,2-a]pyridine-8-carboxamide

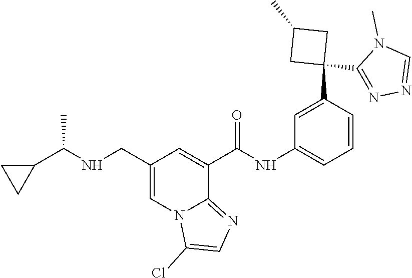 [SMILES: C[C@H](NCc1cc(C(=O)Nc2cccc([C@]3(c4nncn4C)C[C@@H](C)C3)c2)c2ncc(Cl)n2c1)C1CC1]

The compound A58 was synthesized starting from the corresponding starting materials according the similar procedures described as those of Compound A57 (14.59 mg, 28.16 umol, 31.61% yield) was obtained. MS: M/e 518 (M+1)⁺. ¹H NMR (400 MHz, DMSO-d₆) δ ppm 11.95 (s, 1H), 8.52 (s, 1H), 8.30 (s, 1H), 8.24 (s, 1H), 7.94 (s, 1H), 7.73 (s, 1H), 7.66 (d, J=8.12 Hz, 1H), 7.42 (t, J=7.88 Hz, 1H), 7.16 (d, J=7.74 Hz, 1H), 3.84-4.03 (m, 2H), 3.20 (s, 3H), 2.84 (d, J=3.38 Hz, 2H), 2.52-2.61 (m, 3H), 2.33-2.43 (m, 1H), 1.91 (dd, J=8.00, 6.50 Hz, 1H), 1.05-1.12 (m, 6H), 0.64-0.74 (m, 1H), 0.40-0.50 (m, 1H), 0.29-0.37 (m, 1H), 0.20 (d, J=9.20, 4.73 Hz, 1H), 0.03 (m, 1H) ppm.

### Compound A59: 6-((((S)-1-cyclopropylethyl)amino)methyl)-3-methyl-N-(3-((1s,3R)-3-methyl-1-(4-methyl-4H-1,2,4-triazol-3-yl)cyclobutyl)phenyl)imidazo[1,2-a]pyridine-8-carboxamide

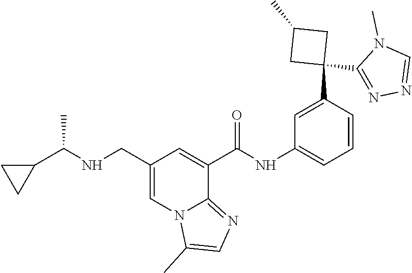 [SMILES: Cc1cnc2c(C(=O)Nc3cccc([C@]4(c5nncn5C)C[C@@H](C)C4)c3)cc(CN[C@@H](C)C3CC3)cn12]

The compound A59 was synthesized starting from the corresponding starting materials according the similar procedures described as those of Compound A46 to give the product (33.69 mg, 29.01% yield). MS: M/e 484 (M+1)⁺; H NMR (400 MHz, DMSO-d₆) δ=12.45 (s, 1H), 8.46 (s, 1H), 8.33-8.28 (m, 1H), 8.22-8.14 (m, 1H), 7.77-7.73 (m, 1H), 7.70-7.62 (m, 1H), 7.58 (d, J=0.8 Hz, 1H), 7.46-7.38 (m, 1H), 7.18-7.10 (m, 1H), 3.98-3.79 (m, 2H), 3.20 (s, 3H), 2.90-2.77 (m, 2H), 2.55 (br d, J=8.0 Hz, 3H), 2.53 (s, 3H), 1.98-1.85 (m, 1H), 1.13-1.08 (m, 6H), 0.77-0.65 (m, 1H), 0.51-0.41 (m, 1H), 0.39-0.29 (m, 1H), 0.26-0.16 (m, 1H), 0.08-0.01 (m, 1H) ppm.

### Compound A60: 6-((((S)-1-cyclobutylethyl)amino)methyl)-3-methyl-N-(3-((1s,3R)-3-methyl-1-(4-methyl-4H-1,2,4-triazol-3-yl)cyclobutyl)phenyl)imidazo[1,2-a]pyridine-8-carboxamide

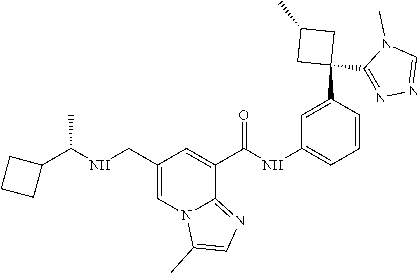 [SMILES: Cc1cnc2c(C(=O)Nc3cccc([C@]4(c5nncn5C)C[C@@H](C)C4)c3)cc(CN[C@@H](C)C3CCC3)cn12]

The compound A60 was synthesized starting from the corresponding starting materials according the similar procedures described as those of Compound A46 to give the product (2.40 mg, 13.40% yield). MS: M/e 512 (M+1)⁺. ¹H NMR (400 MHz, DMSO-d₆) δ=12.46 (s, 1H), 8.46 (s, 1H), 8.30 (s, 1H), 8.18 (d, J=0.8 Hz, 1H), 7.75 (s, 1H), 7.65 (br d, J=8.0 Hz, 1H), 7.58 (s, 1H), 7.41 (t, J=8.0 Hz, 1H), 7.15 (d, J=8.0 Hz, 1H), 4.03-3.66 (m, 2H), 3.20 (s, 3H), 2.84 (br d, J=4.0 Hz, 2H), 2.62-2.51 (m, 6H), 2.48-2.39 (m, 1H), 2.27-2.12 (m, 1H), 2.10-1.57 (m, 7H), 1.09 (br d, J=8.0 Hz, 3H), 0.92 (d, J=8.0 Hz, 3H) ppm.

### Compound A61: N-(3-((1s, 3s)-3-(cyanomethyl)-1-(4-methyl-4H-1,2,4-triazol3yl)cyclobutyl)phenyl)3fluoro6(((1methylcyclobutyl)amino)methyl)imidazo[1,2-a]pyridine-8-carboxamide

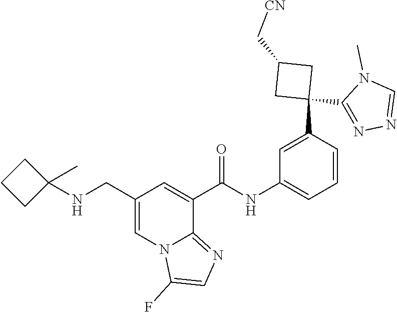 [SMILES: Cn1cnnc1[C@]1(c2cccc(NC(=O)c3cc(CNC4(C)CCC4)cn4c(F)cnc34)c2)C[C@H](CC#N)C1]

### Step 1: N-(3-((1s, 3s)-3-(cyanomethyl)-1-(4-methyl-4H-1,2,4-triazol-3-yl)cyclobutyl)phenyl)-3-fluoro-6-(hydroxymethyl)imidazo[1,2-a]pyridine-8-carboxamide

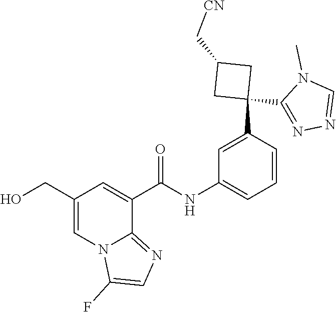 [SMILES: Cn1cnnc1[C@]1(c2cccc(NC(=O)c3cc(CO)cn4c(F)cnc34)c2)C[C@H](CC#N)C1]

A mixture of [3-fluoro-6-(hydroxymethyl)imidazo[1,2-a]pyridine-8-carbonyl]oxylithium (0.2 g, 740.42 umol, 80% purity, 1 eq), 2-((1s, 3s)-3-(3-aminophenyl)-3-(4-methyl-4H-1,2,4-triazol-3-yl)cyclobutyl)acetonitrile (197.93 mg, 740.42 umol, 1 eq), HATU (337.84 mg, 888.50 umol, 1.2 eq), DIEA (287.08 mg, 2.22 mmol, 386.89 uL, 3 eq), DMF (4 mL) was stirred at 25° C. for 2 hrs. The mixture was poured into 50 ml H₂O, extracted with Dichloromethane (30 mL*2), washed with brine (20 mL*2), the combined organic layers were dried over Na₂SO₄, filtered and concentrated under reduced pressure to give a residue. The residue was purified by Prep-HPLC (column: Waters Xbridge BEH C18 100*30 mm*10 um; mobile phase: [water (NH₄HCO₃)-ACN]; B %: 20%-50%, 8 min). Compound N-(3-((1s, 3s)-3-(cyanomethyl)-1-(4-methyl-4H-1,2,4-triazol-3-yl)cyclobutyl)phenyl)-3-fluoro-6-(hydroxymethyl)imidazo[1,2-a]pyridine-8-carboxamide (0.045 g, 97.94 umol, 13.23% yield) was obtained. MS: M/e 460 (M+1)

### Step 2: (8-((3-((1s, 3s)-3-(cyanomethyl)-1-(4-methyl-4H-1,2,4-triazol-3-yl)cyclobutyl)phenyl)carbamoyl)-3-fluoroimidazo[1,2-a]pyridin-6-yl)methyl methanesulfonate

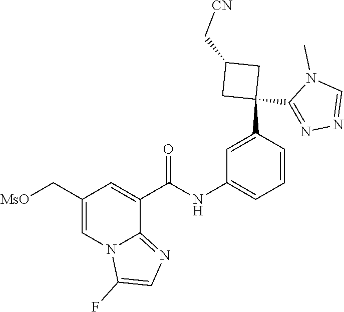 [SMILES: Cn1cnnc1[C@]1(c2cccc(NC(=O)c3cc(COS(C)(=O)=O)cn4c(F)cnc34)c2)C[C@H](CC#N)C1]

In mixture of N-(3-((1s, 3s)-3-(cyanomethyl)-1-(4-methyl-4H-1,2,4-triazol-3-yl)cyclobutyl)phenyl)-3-fluoro-6-(hydroxymethyl)imidazo[1,2-a]pyridine-8-carboxamide (0.045 g, 97.94 umol, 1 eq), TEA (99.10 mg, 979.38 umol, 136.32 uL, 10 eq), DMF (2 mL) was added MsCl (53.85 mg, 470.10 umol, 36.39 uL, 4.8 eq) at 0° C., then the mixture was warmed to 25° C. for 2 hrs. The mixture was diluted with Dichloromethane (20 mL), washed with H₂O (15 mL*3), the combined organic layers were dried over Na₂SO₄, filtered and concentrated under reduced pressure to give a residue. Compound (8-((3-((1s, 3s)-3-(cyanomethyl)-1-(4-methyl-4H-1,2,4-triazol-3-yl)cyclobutyl)phenyl)carbamoyl)-3-fluoroimidazo[1,2-a]pyridin-6-yl)methyl methanesulfonate (0.045 g, crude) was obtained. MS: M/e 538 (M+1)+

### Step 3: N-(3-((1s, 3s)-3-(cyanomethyl)-1-(4-methyl-4H-1,2,4-triazol-3-yl)cyclobutyl)phenyl)-3-fluoro-6-(((1-methylcyclobutyl)amino)methyl)imidazo[1,2-a]pyridine-8-carboxamide

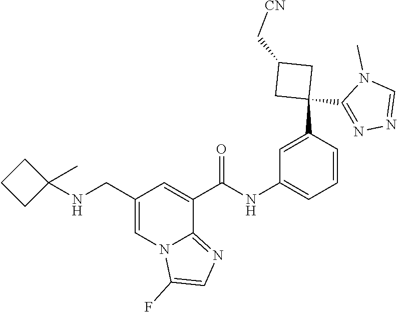 [SMILES: Cn1cnnc1[C@]1(c2cccc(NC(=O)c3cc(CNC4(C)CCC4)cn4c(F)cnc34)c2)C[C@H](CC#N)C1]

In mixture of 1-methylcyclobutanamine (57.25 mg, 470.79 umol, 5 eq, HCl), DMF (2 mL), TEA (47.64 mg, 470.79 umol, 65.53 uL, 5 eq) was added (8-((3-((1s, 3s)-3-(cyanomethyl)-1-(4-methyl-4H-1,2,4-triazol-3-yl)cyclobutyl)phenyl)carbamoyl)-3-fluoroimidazo[1,2-a]pyridin-6-yl)methyl methanesulfonate (0.045 g, 94.16 umol, 1 eq), Cs₂CO₃ (61.36 mg, 188.32 umol, 2 eq). The mixture was stirred at 25° C. for 2 hrs. The solution was purified by Prep-HPLC (column: Waters Xbridge Prep OBD C18 150*40 mm*10 um; mobile phase: [water (NH₄HCO₃)-ACN]; B %: 30%-60%, 8 min). Compound N-(3-((1s, 3s)-3-(cyanomethyl)-1-(4-methyl-4H-1,2,4-triazol-3-yl)cyclobutyl)phenyl)-3-fluoro-6-(((1-methylcyclobutyl)amino)methyl)imidazo[1,2-a]pyridine-8-carboxamide (14.18 mg, 26.93 umol, 28.60% yield) was obtained. MS: M/e 527 (M+1)⁺; ¹H NMR (400 MHz, DMSO-d₆) δ ppm 11.95 (s, 1H), 8.49 (d, J=0.88 Hz, 1H), 8.32 (s, 1H), 8.17 (d, J=1.50 Hz, 1H), 7.72 (d, J=1.63 Hz, 1H), 7.66-7.71 (m, 1H), 7.62 (d, J=6.88 Hz, 1H), 7.44 (t, J=7.94 Hz, 1H), 7.21 (d, J=7.75 Hz, 1H), 3.74 (s, 2H), 3.22 (s, 3H), 2.84-2.94 (m, 2H), 2.66-2.81 (m, 5H), 2.44 (s, 1H), 1.94-2.05 (m, 2H), 1.63-1.80 (m, 4H), 1.26 (s, 3H) ppm.

### Compound A62: 3-fluoro-6-((isobutylamino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

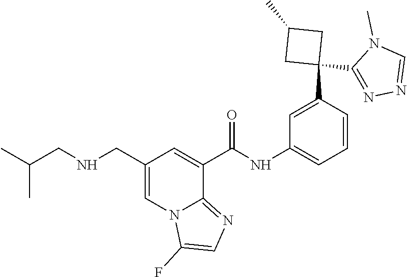 [SMILES: CC(C)CNCc1cc(C(=O)Nc2cccc([C@]3(c4nncn4C)C[C@@H](C)C3)c2)c2ncc(F)n2c1]

### Step 1: (3-fluoro-8-((3-((1s, 3s)-3-methyl-1-(4-methyl-4H-1,2,4-triazol-3-yl)cyclobutyl)phenyl)carbamoyl)imidazo[1,2-a]pyridin-6-yl)methyl methanesulfonate

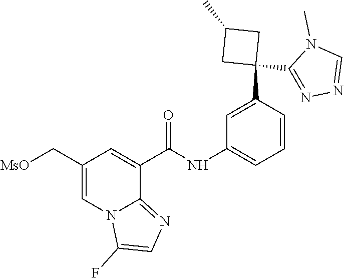 [SMILES: Cn1cnnc1[C@]1(c2cccc(NC(=O)c3cc(COS(C)(=O)=O)cn4c(F)cnc34)c2)C[C@H](C)C1]

To a solution of 3-fluoro-6-(hydroxymethyl)-N-(3-((1s, 3s)-3-methyl-1-(4-methyl-4H-1,2,4-triazol-3-yl)cyclobutyl)phenyl)imidazo[1,2-a]pyridine-8-carboxamide (0.077 g, 177.23 umol) and TEA (143.47 mg, 1.42 mmol) in DMF (3 mL) was added MsCl (101.51 mg, 886.15 umol) at 0° C. The mixture was stirred at 0-25° C. for 2 hours. LCMS showed 3-fluoro-6-(hydroxymethyl)-N-(3-((1s, 3s)-3-methyl-1-(4-methyl-4H-1,2,4-triazol-3-yl)cyclobutyl)phenyl)imidazo[1,2-a]pyridine-8-carboxamide remained. Then the TEA (143.47 mg, 1.42 mmol) and MsCl (101.51 mg, 886.15 umol) was added to the reaction at 0° C. The mixture was stirred at 25° C. for 1 h. The mixture was diluted with DCM (5 mL), then poured into H₂O (2 mL) and extracted with DCM (5 mL×3) and dried over Na₂SO₄, filtered and concentrated to give the crude product. Compound (3-fluoro-8-((3-((1s, 3s)-3-methyl-1-(4-methyl-4H-1,2,4-triazol-3-yl)cyclobutyl)phenyl)carbamoyl)imidazo[1,2-a]pyridin-6-yl)methyl methanesulfonate (90 mg, crude) was used directly into next step. MS: M/e 453 (M+1)⁺.

### Step 2: 3-fluoro-6-((isobutylamino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

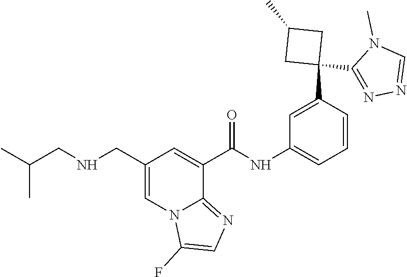 [SMILES: CC(C)CNCc1cc(C(=O)Nc2cccc([C@]3(c4nncn4C)C[C@@H](C)C3)c2)c2ncc(F)n2c1]

To a solution of (3-fluoro-8-((3-((1s, 3s)-3-methyl-1-(4-methyl-4H-1,2,4-triazol-3-yl)cyclobutyl)phenyl)carbamoyl)imidazo[1,2-a]pyridin-6-yl)methyl methanesulfonate (0.09 g, 175.59 umol) and 2-methylpropan-1-amine (64.21 mg, 877.95 umol) in DMF (2 mL) was added Cs₂CO₃ (114.42 mg, 351.18 umol). The mixture was stirred at 25° C. for 12 hrs. The mixture was filtered and purified by Prep-HPLC (column=Waters Xbridge Prep OBD C¹⁸ 150*40 mm*10 um; mobile phase=water (NH4HCO₃)-ACN, B %=35%-65%; 8 min) to give the product (13.47 mg, 15.4% yield). MS: M/e 490 (M+1)⁺. ¹H NMR (400 MHz, DMSO-d₆) δ=11.95 (s, 1H), 8.47 (s, 1H), 8.29 (s, 1H), 8.17 (d, J=1.6 Hz, 1H), 7.71 (s, 1H), 7.68-7.64 (m, 1H), 7.62 (d, J=7.6 Hz, 1H), 7.42 (t, J=7.6 Hz, 1H), 7.17 (d, J=7.6 Hz, 1H), 3.80 (s, 2H), 3.19 (s, 3H), 2.87-2.80 (m, 2H), 2.55 (br d, J=6.4 Hz, 3H), 2.32 (d, J=6.8 Hz, 2H), 1.68 (td, J=6.4, 13.3 Hz, 1H), 1.09 (d, J=5.2 Hz, 3H), 0.87 (d, J=6.4 Hz, 6H) ppm.

### Compound A63: 6-({[(1S)-1-cyclobutylethyl]amino}methyl)-3-methyl-N-{5-[(1r, 3s)-3-methyl-1-(4-methyl-1,2,4-triazol-3-yl)cyclobutyl]pyridin-3-yl}imidazo[1,2-a]pyridine-8-carboxamide

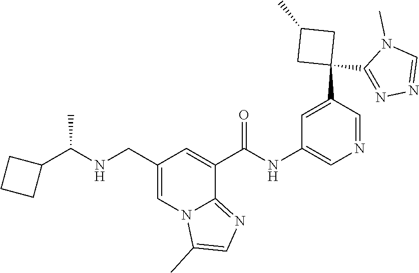 [SMILES: Cc1cnc2c(C(=O)Nc3cncc([C@]4(c5nncn5C)C[C@@H](C)C4)c3)cc(CN[C@@H](C)C3CCC3)cn12]

The compound A63 was synthesized starting from the corresponding starting materials according the similar procedures described as those of Compound A55 to give the product (4.6 mg, 18.51%). MS: M/e 513 (M+1)⁺. ¹H NMR (300 MHz, DMSO-d₆) δ 12.56 (s, 1H), 8.85 (d, J=2.3 Hz, 1H), 8.49 (d, J=1.6 Hz, 1H), 8.40 (d, J=2.1 Hz, 1H), 8.34 (s, 1H), 8.19 (d, J=1.6 Hz, 1H), 8.17-8.12 (m, 1H), 7.59 (d, J=1.2 Hz, 1H), 3.93-3.73 (m, 2H), 3.24 (s, 3H), 2.91 (d, J=3.5 Hz, 2H), 2.59 (d, J=6.6 Hz, 3H), 2.56-2.51 (m, 4H), 2.48 (d, J=5.9 Hz, 1H), 2.21 (s, 1H), 2.07-1.95 (m, 1H), 1.95-1.82 (m, 1H), 1.82-1.61 (m, 4H), 1.10 (d, J=5.0 Hz, 3H), 0.93 (d, J=6.2 Hz, 3H) ppm.

### Compound A64: 6-((((S)-1-cyclopropylethyl)amino)methyl)-3-methyl-N-(5-((1s,3R)-3-methyl-1-(4-methyl-4H-1,2,4-triazol-3-yl)cyclobutyl)pyridin-3-yl)imidazo[1,2-a]pyridine-8-carboxamide

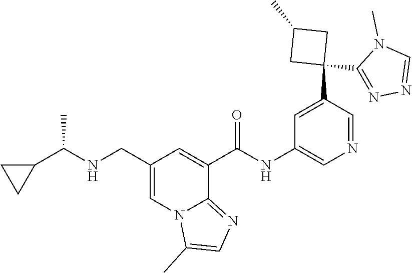 [SMILES: Cc1cnc2c(C(=O)Nc3cncc([C@]4(c5nncn5C)C[C@@H](C)C4)c3)cc(CN[C@@H](C)C3CC3)cn12]

The compound A64 was synthesized starting from the corresponding starting materials according the similar procedures described as those of Compound A55 to give the product (3.0 mg, 8.72%). MS: M/e 499 (M+1)⁺. ¹H NMR (400 MHz, DMSO-d₆) δ 12.56 (s, 1H), 8.85 (m, 1H), 8.49 (m, 1H), 8.41 (m, 1H), 8.34 (s, 1H), 8.19 (m, 1H), 8.15 (m, 1H), 7.59 (s, 1H), 3.90 (m, 2H), 3.24 (s, 3H), 2.92 (m, 2H), 2.59 (m, 3H), 2.54 (s, 3H), 2.48-2.32 (m, 1H), 1.92 (m, 1H), 1.11 (m, 6H), 0.71 (m, 1H), 0.46 (m, 1H), 0.35 (m, 1H), 0.21 (m, 1H), 0.04 (m, 1H) ppm.

### Compound A65: 6-((((S)-1-cyclobutylethyl)amino)methyl)-3-fluoro-N-(3-((1s,3R)-3-methyl-1-(4-methyl-4H-1,2,4-triazol-3-yl)cyclobutyl)phenyl)imidazo[1,2-a]pyridine-8-carboxamide

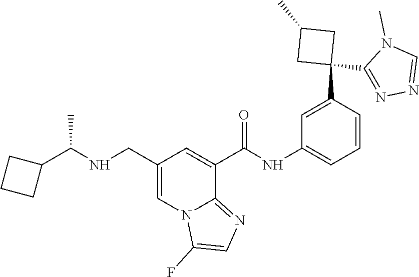 [SMILES: C[C@H](NCc1cc(C(=O)Nc2cccc([C@]3(c4nncn4C)C[C@@H](C)C3)c2)c2ncc(F)n2c1)C1CCC1]

compound A65 was synthesized starting from the corresponding starting materials according the similar procedures described as those of Compound A19 (6.04 mg, 11.71 umol, 15.01% yield) was obtained. MS(ESI) m/e [M+1]516; ¹H NMR (400 MHz, DMSO-d₆) δ ppm 11.84-11.94 (m, 1H) 8.50 (s, 1H) 8.26 (s, 1H) 8.15-8.19 (m, 1H) 7.58-7.67 (m, 3H) 7.35-7.41 (m, 1H) 7.14 (d, J=7.74 Hz, 1H) 3.74-3.95 (m, 2H) 3.15 (s, 3H) 2.80 (d, J=3.00 Hz, 2H) 2.56-2.50 (m, 4H), 2.20 (d, J=2.50 Hz, 1H) 1.93-2.02 (m, 1H) 1.81-1.89 (m, 1H) 1.59-1.79 (m, 5H) 1.05 (d, J=5.00 Hz, 3H) 0.92 (s, 3H) ppm.

### Compound A66: 3-chloro-N-(3-((1s, 3s)-3-methyl-1-(4-methyl-4H-1,2,4-triazol-3-yl)cyclobutyl)phenyl)-6-(((1-methylcyclobutyl)amino)methyl)imidazo[1,2-a]pyridine-8-carboxamide

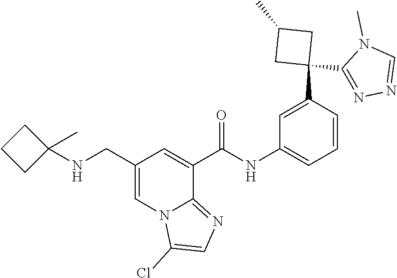 [SMILES: Cn1cnnc1[C@]1(c2cccc(NC(=O)c3cc(CNC4(C)CCC4)cn4c(Cl)cnc34)c2)C[C@H](C)C1]

The compound A66 was synthesized starting from the corresponding starting materials according the similar procedures described as those of Compound A57 to give the product (6.82 mg, 13.16 μmol, 14.77% yield) was obtained. MS: M/e 518 (M+1)⁺. ¹H NMR (400 MHz, DMSO-d₆) δ ppm 11.97 (s, 1H), 8.55 (d, J=1.13 Hz, 1H), 8.30 (s, 1H), 8.25 (d, J=1.50 Hz, 1H), 7.96 (s, 1H), 7.73 (s, 1H), 7.67 (d, J=7.88 Hz, 1H), 7.42 (t, J=7.94 Hz, 1H), 7.17 (d, J=7.88 Hz, 1H), 3.79 (s, 2H), 3.20 (s, 3H), 2.52-2.90 (m, 6H), 1.93-2.11 (m, 2H), 1.61-1.81 (m, 4H), 1.26 (s, 3H), 1.09 (d, J=5.38 Hz, 3H) ppm.

### Compound A67: 3-chloro-N-(3-((1s, 3s)-3-methyl-1-(4-methyl-4H-1,2,4-triazol-3-yl)cyclobutyl)phenyl)-6-(((1-methylcyclopropyl)amino)methyl)imidazo[1,2-a]pyridine-8-carboxamide

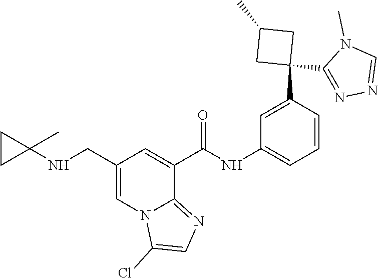 [SMILES: Cn1cnnc1[C@]1(c2cccc(NC(=O)c3cc(CNC4(C)CC4)cn4c(Cl)cnc34)c2)C[C@H](C)C1]

The compound A67 was synthesized starting from the corresponding starting materials according the similar procedures described as those of Compound A57 to give the product (17.27 mg, 34.26 umol, 38.45% yield) was obtained. MS: M/e 504 (M+1)⁺. ¹H NMR (400 MHz, DMSO-d₆) δ 11.96 (s, 1H), 8.48-8.61 (m, 1H), 8.29 (s, 1H), 8.19 (d, J=0.88 Hz, 1H), 7.96 (s, 1H), 7.57-7.77 (m, 2H), 7.34-7.48 (m, 1H), 7.11-7.21 (m, 1H), 3.90 (s, 2H), 3.19 (s, 3H), 2.80-2.87 (m, 2H), 2.55 (d, J=6.00 Hz, 3H), 1.28 (s, 3H), 1.09 (d, J=5.00 Hz, 3H), 0.53 (s, 2H), 0.33 (d, J=1.74 Hz, 2H) ppm.

### Compound A68: 3-chloro-6-(((cyclopentylmethyl)amino)methyl)-N-(3-((1s, 3s)-3-methyl-1-(4-methyl-4H-1,2,4-triazol-3-yl)cyclobutyl)phenyl)imidazo[1,2-a]pyridine-8-carboxamide

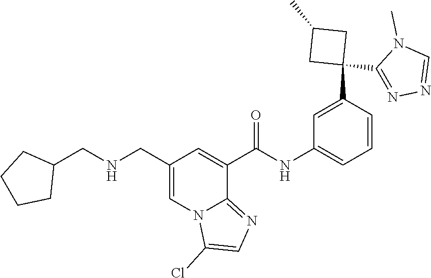 [SMILES: Cn1cnnc1[C@]1(c2cccc(NC(=O)c3cc(CNCC4CCCC4)cn4c(Cl)cnc34)c2)C[C@H](C)C1]

The compound A68 was synthesized starting from the corresponding starting materials according the similar procedures described as those of Compound A57 to give the product (10.14 mg, 19.06 umol, 14.26% yield) was obtained. MS: M/e 532 (M+1)⁺. ¹H NMR (400 MHz, DMSO-d₆) δ=11.96 (s, 1H), 8.52 (s, 1H), 8.30 (s, 1H), 8.27-8.19 (m, 1H), 8.03 (s, 1H), 7.77-7.69 (m, 1H), 7.66 (d, J=8.0 Hz, 1H), 7.42 (t, J=8.0 Hz, 1H), 7.22-7.13 (m, 1H), 3.92-3.82 (m, 2H), 3.19 (s, 3H), 2.84 (d, J=3.2 Hz, 2H), 2.55 (d, J=6.4 Hz, 3H), 2.45 (d, J=7.2 Hz, 2H), 2.05-1.88 (m, 1H), 1.77-1.64 (m, 2H), 1.59-1.49 (m, 2H), 1.49-1.40 (m, 2H), 1.27-1.12 (m, 2H), 1.09 (d, J=5.2 Hz, 3H) ppm.

### Compound A69: 3-chloro-6-((((S)-1-cyclobutylethyl)amino)methyl)-N-(3-((1s,3R)-3-methyl-1-(4-methyl-4H-1,2,4-triazol-3-yl)cyclobutyl)phenyl)imidazo[1,2-a]pyridine-8-carboxamide

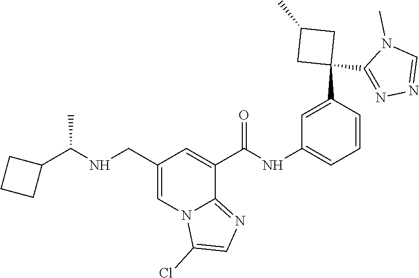 [SMILES: C[C@H](NCc1cc(C(=O)Nc2cccc([C@]3(c4nncn4C)C[C@@H](C)C3)c2)c2ncc(Cl)n2c1)C1CCC1]

The compound A69 was synthesized starting from the corresponding starting materials according the similar procedures described as those of Compound A57 to give the product (12.7 mg, 23.87 umol, 17.86% yield) was obtained. MS: M/e 532 (M+1), ¹H NMR (400 MHz, DMSO-d₆) δ=12.01-11.92 (m, 1H), 8.54 (s, 1H), 8.30 (s, 1H), 8.24 (d, J=0.9 Hz, 1H), 8.03 (s, 1H), 7.75-7.69 (m, 1H), 7.67 (d, J=8.0 Hz, 1H), 7.47-7.35 (m, 1H), 7.22-7.12 (m, 1H), 3.94-3.78 (m, 2H), 3.19 (s, 3H), 2.84 (d, J=3.2 Hz, 2H), 2.60-2.52 (m, 3H), 2.49-2.44 (m, 1H), 2.25-2.12 (m, 1H), 2.06-1.93 (m, 1H), 1.92-1.82 (m, 2H), 1.81-1.71 (m, 2H), 1.71-1.59 (m, 2H), 1.09 (d, J=4.8 Hz, 3H), 0.92 (d, J=6.4 Hz, 3H) ppm.

### Compound A70: 6-((((S)-1-cyclobutylethyl)amino)methyl)-N-(3-((1s,3R)-3-methyl-1-(4-methyl-4H-1,2,4-triazol-3-yl)cyclobutyl)phenyl)imidazo[1,2-a]pyridine-8-carboxamide

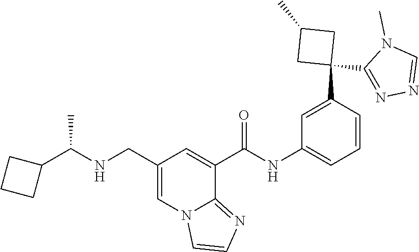 [SMILES: C[C@H](NCc1cc(C(=O)Nc2cccc([C@]3(c4nncn4C)C[C@@H](C)C3)c2)c2nccn2c1)C1CCC1]

### Step A: methyl (S)-6-(((1-cyclobutylethyl)amino)methyl)imidazo[1,2-a]pyridine-8-carboxylate

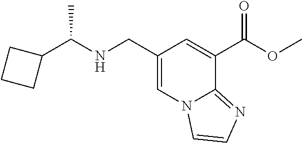 [SMILES: COC(=O)c1cc(CN[C@@H](C)C2CCC2)cn2ccnc12]

A solution of methyl 6-formylimidazo[1,2-a]pyridine-8-carboxylate (50 mg, 0.25 mmol), (S)-1-cyclobutylethan-1-amine hydrochloride (51 mg, 0.38 mmol) and TEA (38 mg, 0.38 mmol) in DCM (2 mL) was stirred at r.t for 30 mins. After cooled under ice bath, NaBH(OAc)₃ (81 mg, 0.38 mmol) was added and followed with AcOH (0.1 mL). The mixture was stirred at r.t overnight. It was added with water (2 mL), basified with NaHCO₃ solution and extracted with DCM (8 mL). The organic layer was dried, concentrated and purified by prep. TLC (DCM:MeOH=10:1) to get the product (34 mg, 49%). MS: M/e 288 (M+1)+

### Step B: methyl (S)-6-(((tert-butoxycarbonyl)(1-cyclobutylethyl)amino)methyl) imidazo[1,2-a]pyridine-8-carboxylate

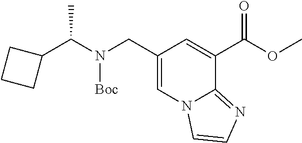 [SMILES: COC(=O)c1cc(CN(C(=O)OC(C)(C)C)[C@@H](C)C2CCC2)cn2ccnc12]

To a solution of methyl (S)-6-(((1-cyclobutylethyl)amino)methyl)imidazo[1,2-a]pyridine-8-carboxylate (34 mg, 0.12 mmol) in DCM (2 mL) was added with (Boc)₂O (39 mg, 0.18 mmol), followed with TEA (19 mg, 0.18 mmol) and DMAP (2 mg, 0.01 mmol). The solution was stirred at r.t overnight. It was concentrated and purified by prep. TLC (EA) to get the product (11 mg, 24%). MS: M/e 388 (M+1)+

### Step C: (S)-6-(((tert-butoxycarbonyl)(1-cyclobutylethyl)amino)methyl)imidazo[1,2-a]pyridine-8-carboxylic acid

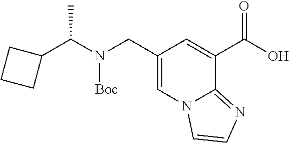 [SMILES: C[C@@H](C1CCC1)N(Cc1cc(C(=O)O)c2nccn2c1)C(=O)OC(C)(C)C]

LiOH·H₂O solution (5 mg, 0.1 mmol, in 1 mL of water) was added to a solution of methyl (S)-6-(((tert-butoxycarbonyl)(1-cyclobutylethyl)amino)methyl)imidazo[1,2-a]pyridine-8-carboxylate (11 mg, 0.03 mmol) in MeOH (2 mL) and it was stirred at r.t for 4 hrs. The reaction mixture was evaporated, dissolved in water (2 mL) and lyophilized to get the product (11 mg, crude). MS: M/e 374 (M+1)+

### Step D: 6-((((S)-1-cyclobutylethyl)amino)methyl)-N-(3-((1s,3R)-3-methyl-1-(4-methyl-4H-1,2,4-triazol-3-yl)cyclobutyl)phenyl)imidazo[1,2-a]pyridine-8-carboxamide

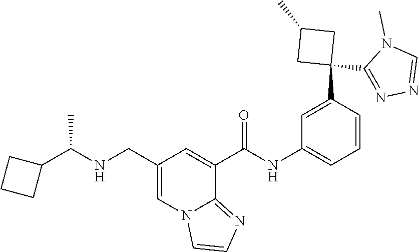 [SMILES: C[C@H](NCc1cc(C(=O)Nc2cccc([C@]3(c4nncn4C)C[C@@H](C)C3)c2)c2nccn2c1)C1CCC1]

A solution of (S)-6-(((tert-butoxycarbonyl)(1-cyclobutylethyl)amino)methyl) imidazo[1,2-a]pyridine-8-carboxylic acid (II mg, 0.03 mmol) and 3-((1s, 3s)-3-methyl-1-(4-methyl-4H-1,2,4-triazol-3-yl)cyclobutyl)aniline (8 mg, 0.03 mmol) in DMF (1 mL) were added with HATU (19 mg, 0.05 mmol) and DIEA (8 mg, 0.06 mmol). The reaction mixture was stirred at r.t for 1 hour, then added with water (3 mL), extracted with DCM (5 mL) and washed with brine (5 mL). The organic layer was dried, concentrated to get the intermediate. It was dissolved in DCM (3 mL) and added with HCl/dioxane (2M, 0.5 mL). After stirred for 2 hrs, the solution was concentrated and purified by prep. HPLC (Mobile Phase A: 0.1% FA-H₂O, Mobile Phase B: 0.1% FA-ACN) to get the product (7 mg, 48%). ¹H NMR (400 MHz, CD₃OD) δ 8.70 (s, 1H), 8.29 (s, 1H), 8.23 (s, 1H), 7.98 (s, 1H), 7.89 (s, 1H), 7.74 (m, 1H), 7.68 (d, J=8.0 Hz, 1H), 7.42 (t, J=8.0 Hz, 1H), 7.17 (d, J=8.0 Hz, 1H), 4.11-3.98 (m, 2H), 3.32 (s, 3H), 2.96-2.92 (m, 3H), 2.67-2.64 (m, 1H), 2.61-2.56 (m, 2H), 2.44-2.38 (m, 1H), 2.16-2.13 (m, 1H), 2.05-2.01 (m, 1H), 1.94-1.77 (m, 4H), 1.14 (t, J=4.0 Hz, 6H) ppm. MS: M/e 498 (M+1)+

### Compound A71: 6-{[(cyclobutylmethyl)amino]methyl}-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide

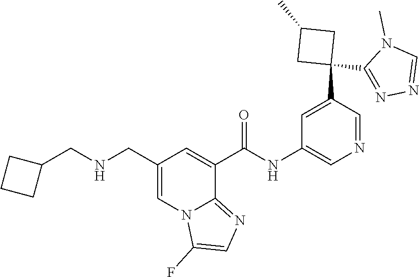 [SMILES: Cn1cnnc1[C@]1(c2cncc(NC(=O)c3cc(CNCC4CCC4)cn4c(F)cnc34)c2)C[C@H](C)C1]

### Step 1: methyl 6-{[(cyclobutylmethyl)amino]methyl}-3-fluoroimidazo[1,2-a]pyridine-8-carboxylate

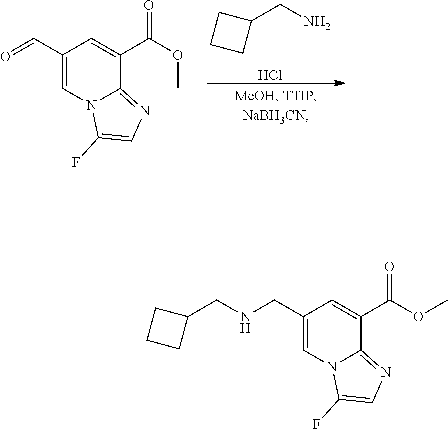

To a stirred mixture of methyl 3-fluoro-6-formylimidazo[1,2-a]pyridine-8-carboxylate (250 mg, 0.994 mmol, 1 equiv, 88.3%) and 1-cyclobutylmethanamine hydrochloride (255 mg, 1.992 mmol, 2.00 equiv, 95%) in MeOH (10 mL, 99%) was added Titanium(IV) isopropoxide (1.2 g, 4.011 mmol, 4.04 equiv, 95%) dropwise at room temperature. The resulting mixture was stirred for 3 hrs at room temperature. To the above mixture was added NaBH₃CN (132 mg, 1.996 mmol, 2.01 equiv, 95%) in portions at room temperature. The resulting mixture was stirred for additional 3 hrs at room temperature. The resulting mixture was concentrated under vacuum. The residue was purified by silica gel column chromatography, eluted with CH₂Cl₂/MeOH (8:1) to afford methyl 6-{[(cyclobutylmethyl)amino]methyl}-3-fluoroimidazo[1,2-a]pyridine-8-carboxylate (160 mg, 44.00%). MS: M/e 292 (M+1)⁺.

### Step 2: 6-{[(cyclobutylmethyl)amino]methyl}-3-fluoroimidazo[1,2-a]pyridine-8-carboxylic acid

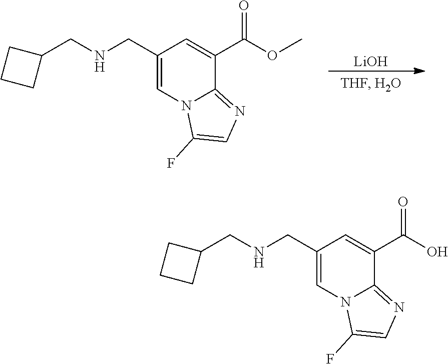 [SMILES: COC(=O)c1cc(CNCC2CCC2)cn2c(F)cnc12 ; "LiOH" ; O=C(O)c1cc(CNCC2CCC2)cn2c(F)cnc12]

To a stirred solution of methyl 6-{[(cyclobutylmethyl)amino]methyl}-3-fluoroimidazo[1,2-a]pyridine-8-carboxylate (145 mg, 0.396 mmol, 1 equiv, 79.6%) in THF (3 mL, 99%) and H₂O (1.5 mL, 99%) was added LiOH (20 mg, 0.793 mmol, 2.00 equiv, 95%) in portions. The resulting mixture was stirred for 3 hrs at room temperature. The mixture was acidified to pH=3 with HCl (1 M). The resulting mixture was concentrated under vacuum. The residue was purified by reversed-phase flash chromatography with the following conditions: column, C18 silica gel; mobile phase, MeCN in Water (0.1% FA), 0%; detector, UV 254 nm. This resulted in 6-{[(cyclobutylmethyl)amino]methyl}-3-fluoroimidazo[1,2-a]pyridine-8-carboxylic acid (40 mg, 36.05%). MS: M/e 278 (M+1)⁺.

### Step 3: 6-{[(cyclobutylmethyl)amino]methyl}-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide

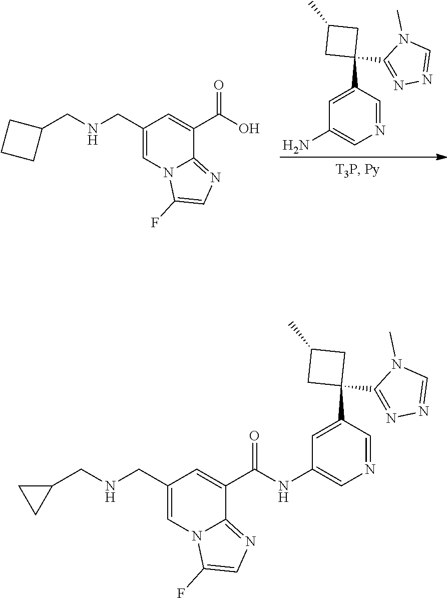 [SMILES: O=C(O)c1cc(CNCC2CCC2)cn2c(F)cnc12 ; Cn1cnnc1[C@]1(c2cncc(N)c2)C[C@H](C)C1 ; Cn1cnnc1[C@]1(c2cncc(NC(=O)c3cc(CNCC4CC4)cn4c(F)cnc34)c2)C[C@H](C)C1]

To a stirred mixture of 6-{[(cyclobutylmethyl)amino]methyl}-3-fluoroimidazo[1,2-a]pyridine-8-carboxylic acid (25 mg, 0.089 mmol, 1 equiv, 99.0%) and 5-[(1r, 3s)-3-methyl-1-(4-methyl-1,2,4-triazol-3-yl)cyclobutyl]pyridin-3-amine (23 mg, 0.090 mmol, 1.01 equiv, 95.0%) in Pyridine (1.5 mL, 95%) were added T₃P (568 mg, 0.893 mmol, 10.00 equiv, 50%) dropwise at room temperature. The resulting mixture was stirred for 30 min at room temperature. The resulting mixture was concentrated under vacuum. The crude product (100 mg) was purified by Prep-HPLC with the following conditions (2 #SHIMADZU (HPLC-01)): Column, YMC-Actus Triart C18 ExRS, 30*150 mm, 5 μm; mobile phase, 10 mmol NH₄HCO₃+0.05% NH₃H2O and ACN (15% ACN up to 45% in 8 min); This resulted in 6-{[(cyclobutylmethyl)amino]methyl}-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide (12.4 mg, 26.45%). MS: M/e 503 (M+1)⁺. ¹H NMR (400 MHz, DMSO-d₆) δ 12.02 (s, 1H), 8.85 (d, J=2.3 Hz, 1H), 8.48 (d, J=1.6 Hz, 1H), 8.42 (d, J=2.1 Hz, 1H), 8.35 (s, 1H), 8.16 (d, J=1.6 Hz, 1H), 8.13-8.08 (m, 1H), 7.62 (d, J=6.9 Hz, 1H), 3.79 (s, 2H), 3.24 (s, 3H), 2.95-2.85 (m, 2H), 2.64-2.56 (m, 3H), 2.54 (d, J=7.2 Hz, 2H), 2.48-2.36 (m, 1H), 2.05-1.93 (m, 2H), 1.91-1.71 (m, 2H), 1.70-1.56 (m, 2H), 1.10 (d, J=5.0 Hz, 3H) ppm.

### Compound A72: 6-({[(1S)-1-cyclopropylethyl]amino}methyl)-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide

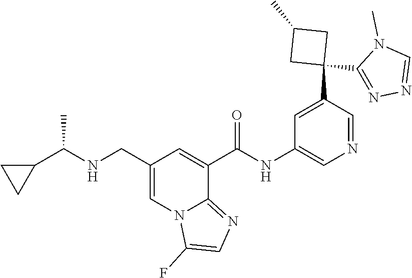 [SMILES: C[C@H](NCc1cc(C(=O)Nc2cncc([C@]3(c4nncn4C)C[C@@H](C)C3)c2)c2ncc(F)n2c1)C1CC1]

### Step 1: methyl 6-({[(1S)-1-cyclopropylethyl]amino}methyl)-3-fluoroimidazo[1,2-a]pyridine-8-carboxylate

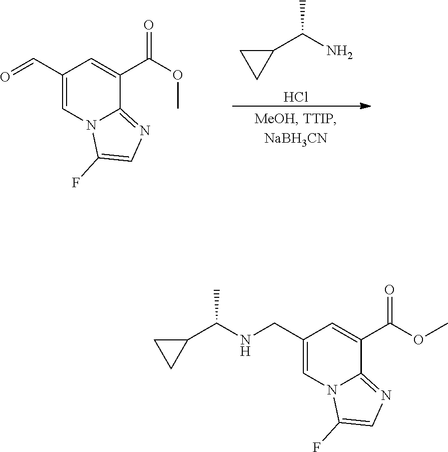

To a stirred mixture of methyl 3-fluoro-6-formylimidazo[1,2-a]pyridine-8-carboxylate (220 mg, 0.874 mmol, 1 equiv, 88.3%) and (1S)-1-cyclopropylethanamine hydrochloride (224 mg, 1.750 mmol, 2.00 equiv, 95%) in MeOH (10 mL, 99%) was added tetrakis(propan-2-yloxy)titanium (1.1 g, 3.677 mmol, 4.21 equiv, 95%) dropwise. The resulting mixture was stirred for 3 hrs at room temperature. To the above mixture was added NaBH₃CN (116 mg, 1.754 mmol, 2.01 equiv, 95%) in portions. The resulting mixture was stirred for additional 3 hrs at room temperature. The resulting mixture was concentrated under vacuum. The residue was purified by silica gel column chromatography, eluted with CH₂Cl₂/MeOH (8:1) to afford methyl 6-({[(1S)-1-cyclopropylethyl]amino}methyl)-3-fluoroimidazo[1,2-a]pyridine-8-carboxylate (150 mg, 45.52%). MS: M/e 292 (M+1)⁺.

### Step 2: 6-({[(1S)-1-cyclopropylethyl]amino}methyl)-3-fluoroimidazo[1,2-a]pyridine-8-carboxylic acid

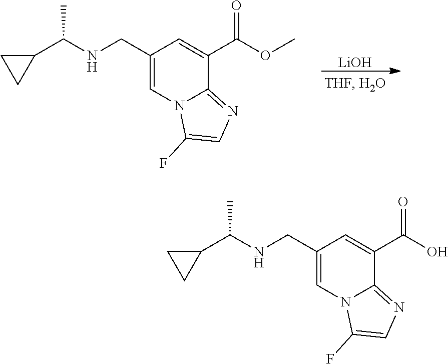

To a stirred solution of methyl 6-({[(1S)-1-cyclopropylethyl]amino}methyl)-3-fluoroimidazo[1,2-a]pyridine-8-carboxylate (145 mg, 0.385 mmol, 1 equiv, 77.3%) in THF (2 mL, 99%) and H₂O (1 mL, 99%) were added LiOH (20 mg, 0.793 mmol, 2.06 equiv, 95%) in portions. The resulting mixture was stirred for 3 hrs at room temperature. The mixture was acidified to pH=4 with HCl (1 M). The resulting mixture was concentrated under vacuum. The residue was purified by reversed-phase flash chromatography with the following conditions: column, C18 silica gel; mobile phase, MeCN in Water (0.1% FA), 0%; detector, UV 254 nm. This resulted in 6-({[(1S)-1-cyclopropylethyl]amino}methyl)-3-fluoroimidazo[1,2-a]pyridine-8-carboxylic acid (45 mg, 37.75%). MS: M/e 278 (M+1)⁺.

### Step 3: 6-({[(1S)-1-cyclopropylethyl]amino}methyl)-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide

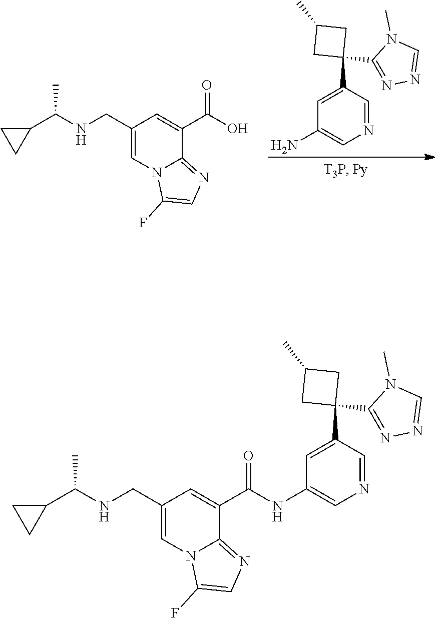 [SMILES: C[C@H](NCc1cc(C(=O)O)c2ncc(F)n2c1)C1CC1 ; Cn1cnnc1[C@]1(c2cncc(N)c2)C[C@H](C)C1 ; C[C@H](NCc1cc(C(=O)Nc2cncc([C@]3(c4nncn4C)C[C@@H](C)C3)c2)c2ncc(F)n2c1)C1CC1]

To a stirred mixture of 6-({[(1S)-1-cyclopropylethyl]amino}methyl)-3-fluoroimidazo[1,2-a]pyridine-8-carboxylic acid (20 mg, 0.065 mmol, 1 equiv, 89.5%) and 5-[(1r, 3s)-3-methyl-1-(4-methyl-1,2,4-triazol-3-yl)cyclobutyl]pyridin-3-amine (17 mg, 0.066 mmol, 1.03 equiv, 95.0%) in Pyridine (1.5 mL, 95%) were added T₃P (411 mg, 0.646 mmol, 10.01 equiv, 50%) dropwise at room temperature. The resulting mixture was stirred for 30 min at room temperature. The resulting mixture was concentrated under vacuum. The crude product (100 mg) was purified by Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (25% ACN up to 55% in 8 min); This resulted in 6-({[(1S)-1-cyclopropylethyl]amino}methyl)-3-fluoro-N-{5-[(1r, 3s)-3-methyl-1-(4-methyl-1,2,4-triazol-3-yl)cyclobutyl]pyridin-3-yl}imidazo[1,2-a]pyridine-8-carboxamide (7.6 mg, 22.86%). MS: M/e 503 (M+1)⁺. ¹H NMR (400 MHz, DMSO-d₆) δ 12.02 (s, 1H), 8.85 (d, J=2.3 Hz, 1H), 8.50 (d, J=1.6 Hz, 1H), 8.42 (d, J=2.1 Hz, 1H), 8.34 (s, 1H), 8.18 (d, J=1.5 Hz, 1H), 8.14-8.08 (m, 1H), 7.62 (d, J=6.9 Hz, 1H), 3.96-3.82 (m, 2H), 3.24 (s, 3H), 2.95-2.85 (m, 2H), 2.64-2.53 (m, 3H), 1.96-1.85 (m, 1H), 1.15-1.07 (m, 6H), 0.76-0.63 (m, 1H), 0.50-0.39 (m, 1H), 0.39-0.29 (m, 1H), 0.25-0.15 (m, 1H), 0.06-0.00 (m, 1H) ppm.

### Compound A73: 3-chloro-N-(5-((1s, 3s)-3-methyl-1-(4-methyl-4H-1,2,4-triazol-3-yl)cyclobutyl)pyridin-3-yl)-6-(((1-methylcyclopropyl)amino)methyl)imidazo[1,2-a]pyridine-8-carboxamide

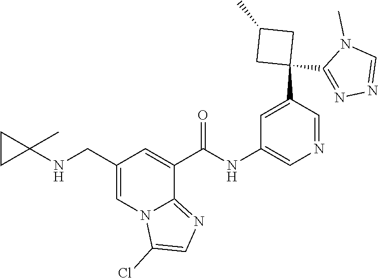 [SMILES: Cn1cnnc1[C@]1(c2cncc(NC(=O)c3cc(CNC4(C)CC4)cn4c(Cl)cnc34)c2)C[C@H](C)C1]

### Step 1: methyl 3-chloro-6-(((1-methylcyclopropyl)amino)methyl)imidazo[1,2-a]pyridine-8-carboxylate

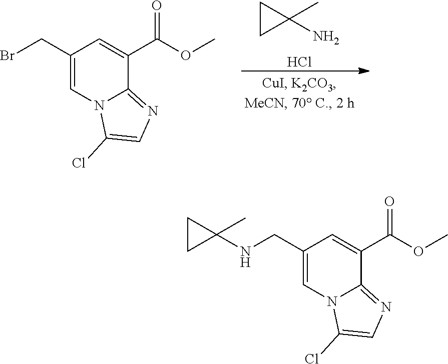

A solution of methyl 6-(bromomethyl)-3-chloroimidazo[1,2-a]pyridine-8-carboxylate (200 mg, 0.652 mmol, 1 equiv, 98.9%), 1-methylcyclopropan-1-amine hydrochloride (150 mg, 1.325 mmol, 2.03 equiv, 95%), NaI (100 mg, 0.634 mmol, 0.97 equiv, 95%) and K₂CO₃ (190 mg, 1.306 mmol, 2.00 equiv, 95%) in MeCN (10 mL, 95%) was stirred for 2 h at 70° C. under nitrogen atmosphere. The mixture was allowed to cool down to room temperature. The resulting mixture was concentrated under reduced pressure. The residue was purified by silica gel column chromatography, eluted with DCM/MeOH (10:1) to afford methyl 3-chloro-6-([(1-methylcyclopropyl)amino]methylimidazo[1,2-a]pyridine-8-carboxylate (106 mg, 43.69%). MS: M/e 294 (M+1)⁺

### Step 2: 3-chloro-6-(((1-methylcyclopropyl)amino)methyl)imidazo[1,2-a]pyridine-8-carboxylic acid

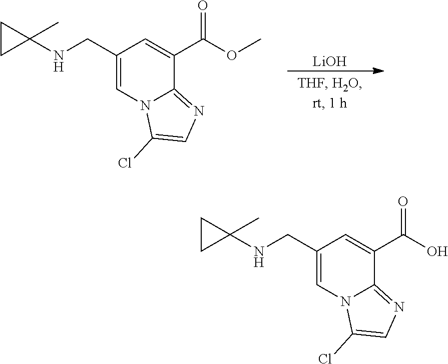 [SMILES: COC(=O)c1cc(CNC2(C)CC2)cn2c(Cl)cnc12 ; "LiOH" ; CC1(NCc2cc(C(=O)O)c3ncc(Cl)n3c2)CC1]

To a stirred solution of methyl 3-chloro-6-([(1-methylcyclopropyl)amino]methylimidazo[1,2-a]pyridine-8-carboxylate (106 mg, 0.285 mmol, 1 equiv, 78.9%) in THF (1.5 mL, 95%) and H₂O (0.5 mL, 26.367 mmol, 92.61 equiv, 95%) was added LiOH (14 mg, 0.555 mmol, 1.95 equiv, 95%) in portions at 0° C. The resulting mixture was stirred for 1 h at room temperature. The mixture was acidified to pH 4 with HCl (3N). The resulting mixture was concentrated under reduced pressure. The residue was purified by reversed-phase flash chromatography with the following conditions: column, C18 silica gel; mobile phase, MeCN in Water (0.1% TFA), 5% to 10% gradient in 4 min; detector, UV 220 nm. This resulted in 3-chloro-6-([(1-methylcyclopropyl)amino]methylimidazo[1,2-a]pyridine-8-carboxylic acid (75 mg, 94.08%).

### Step 3: 3-chloro-N-(5-((1s, 3s)-3-methyl-1-(4-methyl-4H-1,2,4-triazol-3-yl)cyclobutyl)pyridin-3-yl)-6-(((1-methylcyclopropyl)amino)methyl)imidazo[1,2-a]pyridine-8-carboxamide

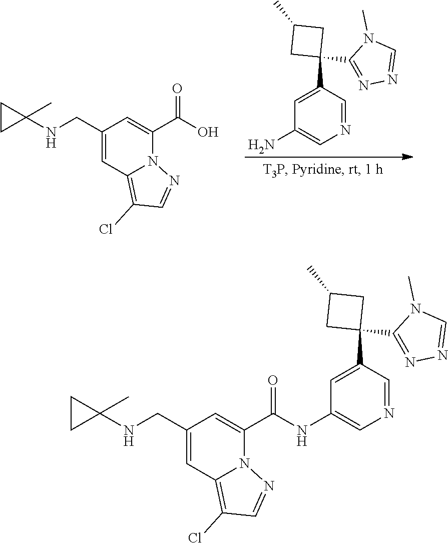 [SMILES: CC1(NCc2cc(C(=O)O)n3ncc(Cl)c3c2)CC1 ; Cn1cnnc1[C@]1(c2cncc(N)c2)C[C@H](C)C1 ; Cn1cnnc1[C@]1(c2cncc(NC(=O)c3cc(CNC4(C)CC4)cc4c(Cl)cnn34)c2)C[C@H](C)C1]

To a stirred solution of 3-chloro-6-([(1-methylcyclopropyl)amino]methylimidazo[1,2-a]pyridine-8-carboxylic acid (75 mg, 0.268 mmol, 1 equiv, 99.9%) and 5-[(1r, 3s)-3-methyl-1-(4-methyl-1,2,4-triazol-3-yl)cyclobutyl]pyridin-3-amine (69 mg, 0.269 mmol, 1.01 equiv, 95%) in Pyridine (2 mL, 95%) was added T₃P (1.70 g, 2.680 mmol, 10 equiv, 50%) dropwise at room temperature. The resulting mixture was stirred for 30 min at room temperature. The resulting mixture was concentrated under reduced pressure. The crude product was purified by Prep-HPLC with the following conditions: Column, XBridge Prep OBD C18 Column, 30×150 mm, 5 μm; mobile phase, 10 mmol NH₄HCO₃+0.05% NH₃·H₂O and ACN (56% ACN up to 86% in 9 min); Detector, 254 nm. This resulted in 3-chloro-6-([(1-methylcyclopropyl)amino]methyl-N-(5-[(1r, 3s)-3-methyl-1-(4-methyl-1,2,4-triazol-3-yl)cyclobutyl]pyridin-3-ylimidazo[1,2-a]pyridine-8-carboxamide (53.3 mg, 37.51). ¹H NMR (400 MHz, DMSO-d₆) δ 12.02 (s, 1H), 8.85 (s, 1H), 8.53 (s, 1H), 8.41 (s, 1H), 8.34 (s, 1H), 8.19 (s, 1H), 8.11 (s, 1H), 7.95 (s, 1H), 3.90 (s, 2H), 3.24 (s, 3H), 2.96-2.86 (m, 2H), 2.64-2.56 (m, 3H), 1.28 (s, 3H), 1.15-1.05 (m, 3H), 0.58-0.50 (m, 2H), 0.37-0.30 (m, 2H) ppm. MS: M/e 505 (M+1)+

### Compound A74: 3-fluoro-N-(5-((1s, 3s)-3-methyl-1-(4-methyl-4H-1,2,4-triazol-3-yl)cyclobutyl)pyridin-3-yl)-6-(((1-methylcyclopropyl)amino)methyl)imidazo[1,2-a]pyridine-8-carboxamide

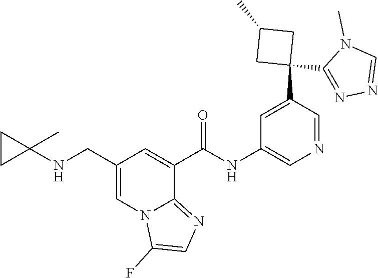 [SMILES: Cn1cnnc1[C@]1(c2cncc(NC(=O)c3cc(CNC4(C)CC4)cn4c(F)cnc34)c2)C[C@H](C)C1]

### Step 1: methyl 6-(bromomethyl)-3-fluoroimidazo[1,2-a]pyridine-8-carboxylate

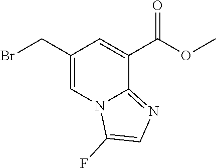 [SMILES: COC(=O)c1cc(CBr)cn2c(F)cnc12]

To a stirred solution of methyl 3-fluoro-6-(hydroxymethyl)imidazo[1,2-a]pyridine-8-carboxylate (200 mg) in CHCl3 (5 mL, 99%) added PBr₃ (64 mg) in portions at 0° C. The resulting mixture was stirred for 3 h at room temperature. The resulting mixture was concentrated under vacuum. The residue was purified by silica gel column chromatography, eluted with CH₂Cl₂/MeOH (7:1) to afford the product (140 mg). MS: M/e 287 (M+1)⁺.

### Step 2: methyl 3-fluoro-6-(((1-methylcyclopropyl)amino)methyl)imidazo[1,2-a]pyridine-8-carboxylate

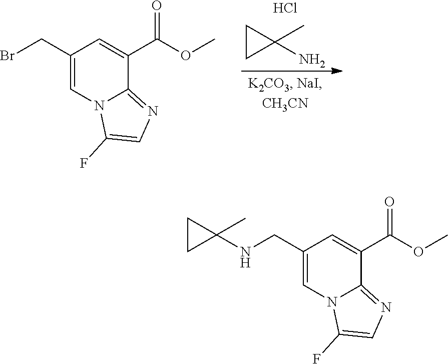

Into a 10 mL sealed tube were added methyl 6-(bromomethyl)-3-fluoroimidazo[1,2-a]pyridine-8-carboxylate (30 mg, 0.103 mmol, 1 equiv, 98.6%) and 1-methylcyclopropan-1-amine hydrochloride (24 mg, 0.212 mmol, 2.06 equiv, 95%), NaI (17 mg, 0.108 mmol, 1.05 equiv, 95%), K₂CO₃ (30 mg, 0.206 mmol, 2.00 equiv, 95%), CH₃CN (1 mL, 99%). The resulting mixture was stirred for 3 h at 70° C. The mixture was allowed to cool down to room temperature. The resulting mixture was concentrated under vacuum. The residue was purified by silica gel column chromatography, eluted with CH₂Cl₂/MeOH (7:1) to afford methyl 3-fluoro-6-([(1-methylcyclopropyl)amino]methylimidazo[1,2-a]pyridine-8-carboxylate (28 mg, 85.75%). MS: M/e 278 (M+1)⁺.

### Step 3: 3-fluoro-6-(((1-methylcyclopropyl)amino)methyl)imidazo[1,2-a]pyridine-8-carboxylic acid

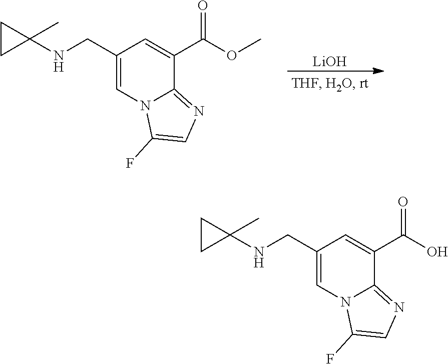 [SMILES: COC(=O)c1cc(CNC2(C)CC2)cn2c(F)cnc12 ; "LiOH" ; CC1(NCc2cc(C(=O)O)c3ncc(F)n3c2)CC1]

To a stirred solution of methyl 3-fluoro-6-([(1-methylcyclopropyl)amino]methylimidazo[1,2-a]pyridine-8-carboxylate (28 mg, 0.088 mmol, 1 equiv, 87.5%) in THF (1.5 mg, 99%) and H₂O (0.5 mL, 99%) were added LiOH (5 mg, 0.198 mmol, 2.24 equiv, 95%) in portions. The resulting mixture was stirred for 1 h at room temperature. The mixture was acidified to pH 3 with HCl (1 M). The resulting mixture was concentrated under vacuum. The residue was purified by reversed-phase flash chromatography with the following conditions: column, C18 silica gel; mobile phase, MeCN in Water (0.1% TFA), 0%; This resulted in 3-fluoro-6-([(1-methylcyclopropyl)amino]methylimidazo[1,2-a]pyridine-8-carboxylic acid (22 mg, 82.28%). MS: M/e 264 (M+1)⁺.

### Step 4: 3-fluoro-N-(5-((1s, 3s)-3-methyl-1-(4-methyl-4H-1,2,4-triazol-3-yl)cyclobutyl)pyridin-3-yl)-6-(((1-methylcyclopropyl)amino)methyl)imidazo[1,2-a]pyridine-8-carboxamide

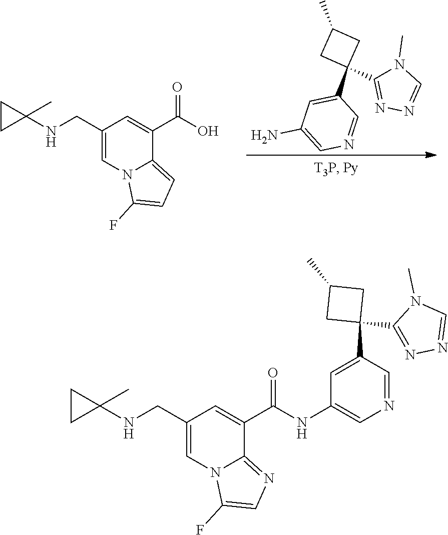 [SMILES: CC1(NCc2cc(C(=O)O)c3ccc(F)n3c2)CC1 ; Cn1cnnc1[C@]1(c2cncc(N)c2)C[C@H](C)C1 ; Cn1cnnc1[C@]1(c2cncc(NC(=O)c3cc(CNC4(C)CC4)cn4c(F)cnc34)c2)C[C@H](C)C1]

To a stirred mixture of 3-fluoro-6-(((1-methylcyclopropyl)amino)methyl)imidazo[1,2-a]pyridine-8-carboxylic acid (19 mg, 0.063 mmol, 1 equiv, 87.0%) and 5-[(1r, 3s)-3-methyl-1-(4-methyl-1,2,4-triazol-3-yl)cyclobutyl]pyridin-3-amine (16 mg, 0.062 mmol, 0.99 equiv, 95.0%) in Pyridine (2 mL, 95%) were added T₃P (400 mg, 0.629 mmol, 10.01 equiv, 50%) dropwise. The resulting mixture was stirred for 30 min at room temperature. The resulting mixture was concentrated under vacuum. The crude product (200 mg) was purified by Prep-HPLC with the following conditions (2 #SHIMADZU (HPLC-01)): Column, XBridge Prep OBD C18 Column, 30*150 mm, 5 μm; mobile phase, 10 mmol NH₄HCO₃+0.05% NH₃H2O and ACN (5% ACN up to 35% in 8 min); This resulted in 3-fluoro-6-([(1-methylcyclopropyl)amino]methyl-N-(5-[(1r, 3s)-3-methyl-1-(4-methyl-1,2,4-triazol-3-yl)cyclobutyl]pyridin-3-ylimidazo[1,2-a]pyridine-8-carboxamide (9.7 mg, 31.05%). MS: M/e 489 (M+1). ¹H NMR (300 MHz, DMSO-d₆) δ 12.01 (s, 1H), 8.85 (d, J=2.3 Hz, 1H), 8.53 (s, 1H), 8.42 (d, J=2.1 Hz, 1H), 8.35 (s, 1H), 8.15-8.08 (m, 2H), 7.63 (d, J=6.9 Hz, 1H), 3.91 (s, 2H), 3.24 (s, 3H), 2.91 (d, J=3.8 Hz, 2H), 2.59 (d, J=6.6 Hz, 3H), 1.44 (s, 3H), 1.13 (m, 3H), 0.62-0.54 (m, 2H), 0.41-0.33 (m, 2H) ppm.

### Compound A75: N-(5-((1s, 3s)-3-methyl-1-(4-methyl-4H-1,2,4-triazol-3-yl)cyclobutyl)pyridin-3-yl)-6-(((1-methylcyclopropyl)amino)methyl)imidazo[1,2-a]pyridine-8-carboxamide

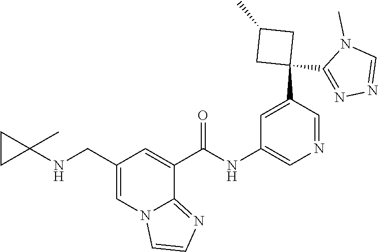 [SMILES: Cn1cnnc1[C@]1(c2cncc(NC(=O)c3cc(CNC4(C)CC4)cn4ccnc34)c2)C[C@H](C)C1]

### Step 1: 6-(((1-methylcyclopropyl)amino)methyl)imidazo[1,2-a]pyridine-8-carboxylic acid

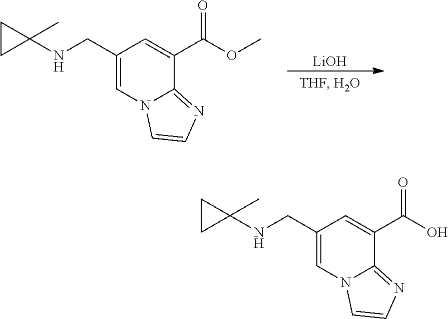 [SMILES: COC(=O)c1cc(CNC2(C)CC2)cn2ccnc12 ; "LiOH" ; CC1(NCc2cc(C(=O)O)c3nccn3c2)CC1]

To a stirred solution of methyl 6-(((1-methylcyclopropyl)amino)methyl)imidazo[1,2-a]pyridine-8-carboxylate (90 mg, 0.330 mmol, 1 equiv, 95%) in THF (1.5 mL, 95%) and H₂O (0.5 mL, 26.367 mmol, 79.97 equiv, 95%) was added LiOH (17 mg, 0.674 mmol, 2.05 equiv, 95%) in portions at 0° C. The resulting mixture was stirred for 1 h at room temperature. The mixture was acidified to pH 5 with HCl (aq.). The resulting mixture was concentrated under reduced pressure. The residue was purified by reversed-phase flash chromatography with the following conditions: column, C18 silica gel; mobile phase, MeCN in Water (0.1% TFA), 0% gradient in 4 min; to give the product (80 mg, 71.12%). MS: M/e 246 (M+1)⁺.

### Step 2: N-(5-((1s, 3s)-3-methyl-1-(4-methyl-4H-1,2,4-triazol-3-yl)cyclobutyl)pyridin-3-yl)-6-(((1-methylcyclopropyl)amino)methyl)imidazo[1,2-a]pyridine-8-carboxamide

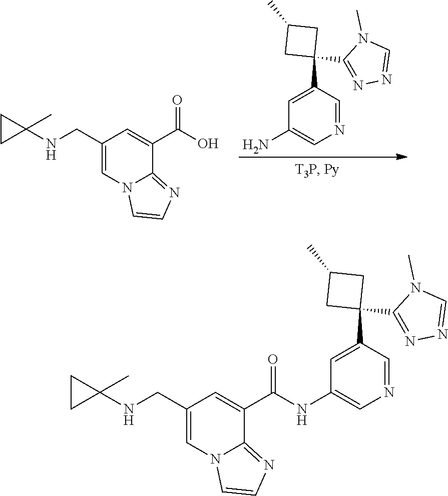 [SMILES: CC1(NCc2cc(C(=O)O)c3nccn3c2)CC1 ; Cn1cnnc1[C@]1(c2cncc(N)c2)C[C@H](C)C1 ; Cn1cnnc1[C@]1(c2cncc(NC(=O)c3cc(CNC4(C)CC4)cn4ccnc34)c2)C[C@H](C)C1]

To a stirred solution of methyl 6-(((1-methylcyclopropyl)amino)methyl)imidazo[1,2-a]pyridine-8-carboxylate (88 mg, 0.258 mmol, 1 equiv, 71.9%) and 5-[(1r, 3s)-3-methyl-1-(4-methyl-1,2,4-triazol-3-yl)cyclobutyl]pyridin-3-amine (65 mg, 0.254 mmol, 0.98 equiv, 95.0%) in Pyridine (3 mL, 95%) was added T₃P (1.64 g, 2.580 mmol, 10 equiv, 50%) dropwise at room temperature. The resulting mixture was stirred for 30 min at room temperature. The resulting mixture was concentrated under reduced pressure. The crude product was purified by Prep-HPLC with the following conditions (2 #SHIMADZU (HPLC-01)): Column, XBridge Prep OBD C18 Column, 19*250 mm, 5 μm; mobile phase, 10 mmol NH₄HCO₃+0.05% NH₃H2O and ACN (25% ACN up to 50% in 8 min); This resulted in 6-([(1-methylcyclopropyl)amino]methyl-N-(5-[(1r, 3s)-3-methyl-1-(4-methyl-1,2,4-triazol-3-yl)cyclobutyl]pyridin-3-ylimidazo[1,2-a]pyridine-8-carboxamide (45.6 mg, 36.63%). MS: M/e 471 (M+1)⁺. ¹H NMR (300 MHz, DMSO-d₆) δ 12.50 (s, 1H), 8.85 (d, J=2.3 Hz, 1H), 8.77-8.70 (m, 1H), 8.43-8.31 (m, 2H), 8.17-8.08 (m, 3H), 7.78 (d, J=1.4 Hz, 1H), 3.82 (s, 2H), 3.24 (s, 3H), 2.97-2.85 (m, 2H), 2.65-2.54 (m, 3H), 1.27 (s, 3H), 1.10 (d, J=5.1 Hz, 3H), 0.56-0.47 (m, 2H), 0.37-0.28 (m, 2H) ppm.

### Compound A76:3-fluoro-N-(5-((1s, 3s)-3-methyl-1-(4-methyl-4H-1,2,4-triazol-3-yl)cyclobutyl)pyridin-3-yl)-6-(((1-methylcyclobutyl)amino)methyl)imidazo[1,2-a]pyridine-8-carboxamide

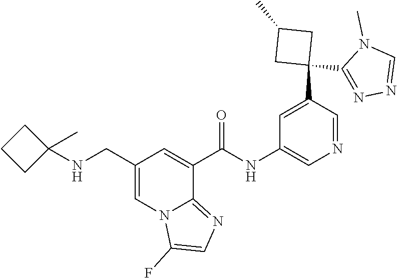 [SMILES: Cn1cnnc1[C@]1(c2cncc(NC(=O)c3cc(CNC4(C)CCC4)cn4c(F)cnc34)c2)C[C@H](C)C1]

### Step A: methyl 3-fluoro-6-(((1-methylcyclobutyl)amino)methyl)imidazo[1,2-a]pyridine-8-carboxylate

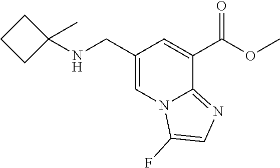 [SMILES: COC(=O)c1cc(CNC2(C)CCC2)cn2c(F)cnc12]

To a solution of methyl 6-(bromomethyl)-3-fluoroimidazo[1,2-a]pyridine-8-carboxylate (100 mg, 0.350 mmol), 1-methylcyclobutan-1-amine hydrochloride (64 mg, 0.524 mmol), K₂CO₃ (145 mg, 1.050 mmol) and NaI (53 mg, 0.350 mmol) in CH₃CN (3 mL) was stirred at room temperature for 6 hours. After completed, the reaction was quenched with aq NH₄Cl (3 mL) and extracted with EA (2×15 mL), dried over Na₂SO₄ and concentrated under vacuum. The residue was purified by silica gel column chromatography, eluted with DCM/MeOH (20:1) to afford the titled compound (80 mg, 79%) MS: M/e 292 (M+1)+

### Step B: 3-fluoro-6-(((1-methylcyclobutyl)amino)methyl)imidazo[1,2-a]pyridine-8-carboxylic acid

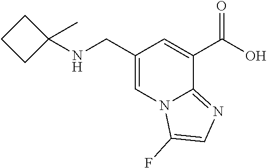 [SMILES: CC1(NCc2cc(C(=O)O)c3ncc(F)n3c2)CCC1]

To a solution of methyl 3-fluoro-6-(((1-methylcyclobutyl)amino)methyl)imidazo[1,2-a]pyridine-8-carboxylate (80 mg, 0.275 mmol) in MeOH (3 ml) and H₂O (1 ml) was added LiOH H₂O (35 mg, 0.825 mmol). The mixture solution was stirred at room temperature for 4 hours. The reaction mixture was concentrated under reduced pressure. Added HCl (2N) to adjust PH-6 and free-dried to give the crude product (80 mg, crude). MS: M/e 278 (M+1)⁺.

### Step C: 3-fluoro-N-(5-((1s, 3s)-3-methyl-1-(4-methyl-4H-1,2,4-triazol-3-yl)cyclobutyl)pyridin-3-yl)-6-(((1-methylcyclobutyl)amino)methyl)imidazo[1,2-a]pyridine-8-carboxamide

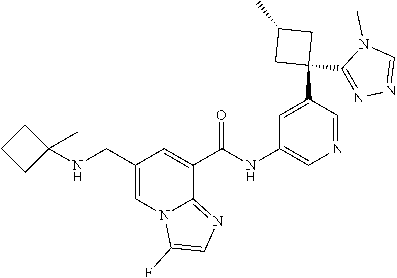 [SMILES: Cn1cnnc1[C@]1(c2cncc(NC(=O)c3cc(CNC4(C)CCC4)cn4c(F)cnc34)c2)C[C@H](C)C1]

To a solution of 3-fluoro-6-(((1-methylcyclobutyl)amino)methyl)imidazo[1,2-a]pyridine-8-carboxylic acid (60 mg, 0.217 mmol), 5-((1s, 3s)-3-methyl-1-(4-methyl-4H-1,2,4-triazol-3-yl)cyclobutyl)pyridin-3-amine (53 mg, 0.217 mmol) in pyridine (3 mL) was added T₃P (414 mg, 0.651 mmol, 50% in EA). The mixture solution was stirred for 2 h at room temperature. Then the reaction solution was quenched with aq NH₄Cl (2 mL) and extracted with EA (2×10 mL), dried over Na₂SO₄ and concentrated under vacuum, which was purified by prep-HPLC to give the title compound (48 mg, 44%). MS: M/e 503 (M+1). ¹H NMR (400 MHz, DMSO-d₆) δ 12.00 (s, 1H), 8.85 (s, 1H), 8.54 (s, 1H), 8.42 (s, 1H), 8.34 (s, 1H), 8.18 (s, 1H), 8.10 (s, 1H), 7.63 (d, J=6.9 Hz, 1H), 3.78 (s, 2H), 3.23 (s, 3H), 2.90 (d, J=3.1 Hz, 2H), 2.57 (t, J=9.7 Hz, 3H), 2.07-1.96 (m, 2H), 1.70 (dt, J=8.7, 5.0 Hz, 4H), 1.28 (s, 3H), 1.09 (d, J=4.8 Hz, 3H) ppm.

### Compound A77: 3-chloro-6-((((S)-1-cyclopropylethyl)amino)methyl)-N-(5-((1s,3R)-3-methyl-1-(4-methyl-4H-1,2,4-triazol-3-yl)cyclobutyl)pyridin-3-yl)imidazo[1,2-a]pyridine-8-carboxamide

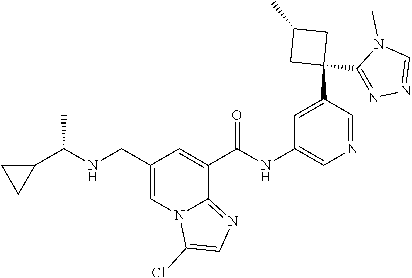 [SMILES: C[C@H](NCc1cc(C(=O)Nc2cncc([C@]3(c4nncn4C)C[C@@H](C)C3)c2)c2ncc(Cl)n2c1)C1CC1]

### Step 1: methyl 3-chloro-6-(hydroxymethyl)imidazo[1,2-a]pyridine-8-carboxylate

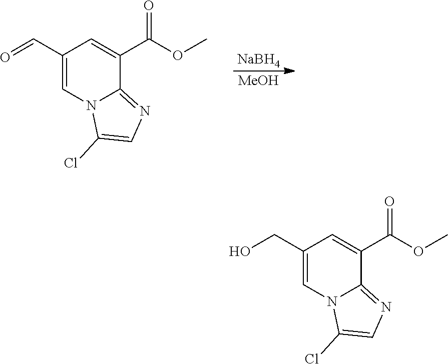 [SMILES: "MeOH" ; COC(=O)c1cc(C=O)cn2c(Cl)cnc12 ; "NaBH4" ; COC(=O)c1cc(CO)cn2c(Cl)cnc12]

To a stirred solution of methyl 3-chloro-6-formylimidazo[1,2-a]pyridine-8-carboxylate (800 mg, 3.282 mmol, 1 equiv, 97.9%) in MeOH was added NaBH₄ (261 mg, 6.554 mmol, 2.00 equiv, 95%) in portions at 0° C. The resulting mixture was stirred for 1 h at room temperature. The resulting mixture was concentrated under reduced pressure. The residue was purified by silica gel column chromatography, eluted with CH2Cl₂/MeOH (10:1) to afford methyl 3-chloro-6-(hydroxymethyl)imidazo[1,2-a]pyridine-8-carboxylate (550 mg, 55.71%). MS: M/e 241 (M+1)⁺.

### Step 2: methyl 6-(bromomethyl)-3-chloroimidazo[1,2-a]pyridine-8-carboxylate

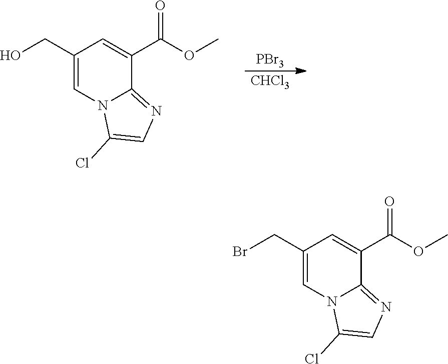

To a stirred solution of methyl 3-chloro-6-(hydroxymethyl)imidazo[1,2-a]pyridine-8-carboxylate (550 mg, 1.828 mmol, 1 equiv, 80.0%) in CHCl₃ (10 mL, 95%) was added PBr₃ (0.27 mL, 2.742 mmol, 1.5 equiv, 95%) dropwise at room temperature. The resulting mixture was stirred for 2 h at 70° C. under nitrogen atmosphere. The resulting mixture was concentrated under reduced pressure. The residue was purified by silica gel column chromatography, eluted with CH₂Cl₂/MeOH (10:1) to afford methyl 6-(bromomethyl)-3-chloroimidazo[1,2-a]pyridine-8-carboxylate (550 mg, 98.01%). MS: M/e 303 (M+1)⁺.

### Step 3: methyl (S)-3-chloro-6-(((1-cyclopropylethyl)amino)methyl)imidazo[1,2-a]pyridine-8-carboxylate

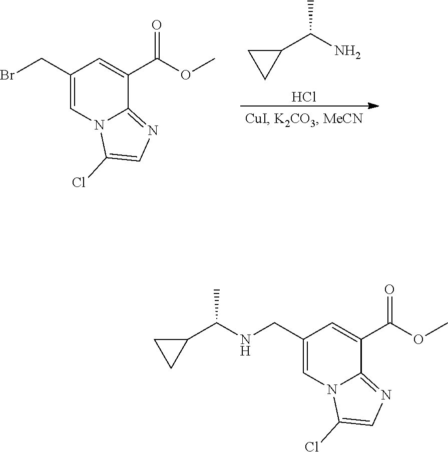

A solution of methyl 6-(bromomethyl)-3-chloroimidazo[1,2-a]pyridine-8-carboxylate (200 mg, 0.652 mmol, 1 equiv, 98.9%), (1S)-1-cyclopropylethanamine hydrochloride (170 mg, 1.328 mmol, 2.04 equiv, 95%), NaI (100 mg, 0.634 mmol, 0.97 equiv, 95%) and K₂CO₃ (190 mg, 1.306 mmol, 2.00 equiv, 95%) in MeCN (10 mL, 95%) was stirred for 2 h at 70° C. under nitrogen atmosphere. The resulting mixture was concentrated under reduced pressure. The residue was purified by silica gel column chromatography, eluted with CH₂Cl₂/MeOH (19:1) to afford methyl methyl (S)-3-chloro-6-(((1-cyclopropylethyl)amino)methyl)imidazo[1,2-a]pyridine-8-carboxylate (150 mg, 61.93%). MS: M/e 308 (M+1)⁺.

### Step 4: (S)-3-chloro-6-(((1-cyclopropylethyl)amino)methyl)imidazo[1,2-a]pyridine-8-carboxylic acid

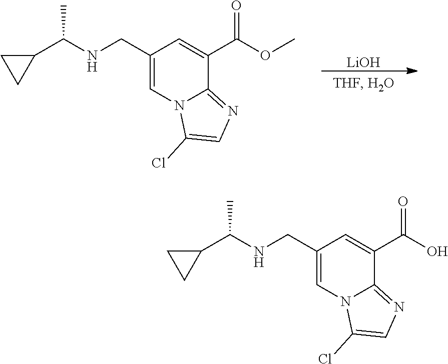

To a stirred solution of methyl 3-chloro-6-(([(1S)-1-cyclopropylethyl]aminomethyl)imidazo[1,2-a]pyridine-8-carboxylate (150 mg, 0.404 mmol, 1 equiv, 82.8%) in THF (3 mL, 95%) and H₂O (1 mL, 52.734 mmol, 130.68 equiv, 95%) was added LiOH (20 mg, 0.793 mmol, 1.97 equiv, 95%) in portions at 0° C. The resulting mixture was stirred for 1 h at room temperature. The mixture was acidified to pH 4 with HCl (aq.). The resulting mixture was concentrated under reduced pressure. The residue was purified by reversed-phase flash chromatography with the following conditions: column, C18 silica gel; mobile phase, MeCN in Water (0.1% TFA), 5% to 10% gradient in 4 min; detector, UV 220 nm. This resulted in (S)-3-chloro-6-(((1-cyclopropylethyl)amino)methyl)imidazo[1,2-a]pyridine-8-carboxylic acid (100 mg, 83.10%). MS: M/e 294 (M+1)⁺.

### Step 5: 3-chloro-6-((((S)-1-cyclopropylethyl)amino)methyl)-N-(5-((1s,3R)-3-methyl-1-(4-methyl-4H-1,2,4-triazol-3-yl)cyclobutyl)pyridin-3-yl)imidazo[1,2-a]pyridine-8-carboxamide

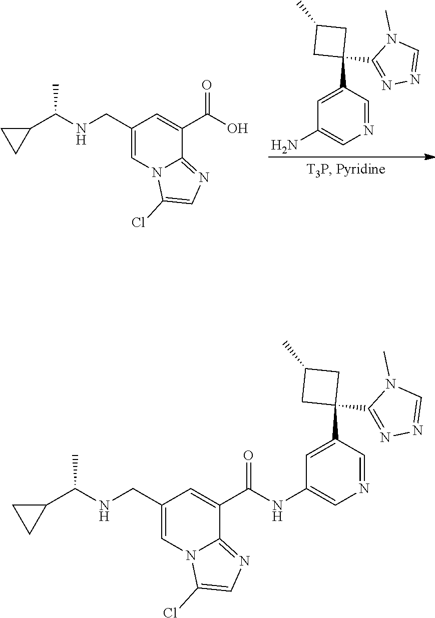 [SMILES: C[C@H](NCc1cc(C(=O)O)c2ncc(Cl)n2c1)C1CC1 ; Cn1cnnc1[C@]1(c2cncc(N)c2)C[C@H](C)C1 ; C[C@H](NCc1cc(C(=O)Nc2cncc([C@]3(c4nncn4C)C[C@@H](C)C3)c2)c2ncc(Cl)n2c1)C1CC1]

To a stirred solution of 3-chloro-6-([(1-methylcyclopropyl)amino]methylimidazo[1,2-a]pyridine-8-carboxylic acid (75 mg, 0.268 mmol, 1 equiv, 99.9%) and 5-[(1r, 3s)-3-methyl-1-(4-methyl-1,2,4-triazol-3-yl)cyclobutyl]pyridin-3-amine (43 mg, 0.168 mmol, 1.00 equiv, 95%) in Pyridine (2 mL, 95%) was added T₃P (1.07 g, 1.680 mmol, 10 equiv, 50%) dropwise at room temperature. The resulting mixture was stirred for 30 min at room temperature. The resulting mixture was concentrated under reduced pressure. The crude product was purified by Prep-HPLC with the following conditions (2 #SHIMADZU (HPLC-01)): Column, XBridge Prep OBD C18 Column, 30*150 mm, 5 μm; mobile phase, 10 mmol NH₄HCO₃+0.05% NH₃H₂O and ACN (56% ACN up to 86% in 9 min); This resulted in 3-chloro-6-(([(1S)-1-cyclopropylethyl]aminomethyl)-N-(5-[(1r, 3s)-3-methyl-1-(4-methyl-1,2,4-triazol-3-yl)cyclobutyl]pyridin-3-ylimidazo[1,2-a]pyridine-8-carboxamide (14.2 mg, 15.88%). MS: M/e 294 (M+1)⁺. ¹H NMR (300 MHz, DMSO-d₆) δ 12.03 (s, 1H), 8.86 (d, J=2.3 Hz, 1H), 8.56 (s, 1H), 8.42 (s, 1H), 8.34 (s, 1H), 8.26 (s, 1H), 8.15-8.08 (m, 1H), 7.96 (s, 1H), 4.04-3.86 (m, 2H), 3.24 (s, 3H), 2.97-2.83 (m, 2H), 2.69-2.52 (m, 4H), 2.00-1.85 (m, 1H), 1.15-1.06 (m, 6H), 0.77-0.61 (m, 1H), 0.52-0.16 (m, 3H) ppm.

### Compound A78: 6-((isobutylamino)methyl)-N-(5-((1s, 3s)-3-methyl-1-(4-methyl-4H-1,2,4-triazol-3-yl)cyclobutyl)pyridin-3-yl)imidazo[1,2-a]pyridine-8-carboxamide

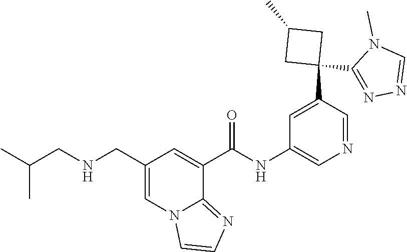 [SMILES: CC(C)CNCc1cc(C(=O)Nc2cncc([C@]3(c4nncn4C)C[C@@H](C)C3)c2)c2nccn2c1]

### Step 1: methyl 6-(hydroxymethyl)imidazo[1,2-a]pyridine-8-carboxylate

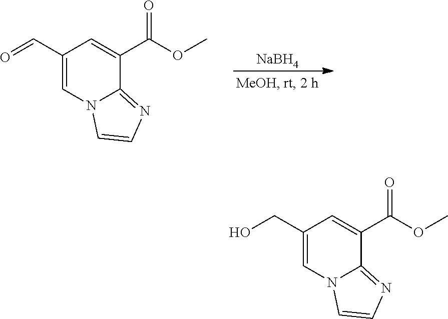

To a stirred solution of methyl 6-formylimidazo[1,2-a]pyridine-8-carboxylate (2.4 g, 7.099 mmol, 1 equiv, 60.4%) in MeOH was added NaBH₄ (566 mg, 14.214 mmol, 2.00 equiv, 95%) in portions at 0° C. The resulting mixture was stirred for additional 3 h at room temperature. The resulting mixture was concentrated under vacuum. The residue was purified by silica gel column chromatography, eluted with CH₂Cl₂/MeOH (8:1) to afford methyl 6-(hydroxymethyl)imidazo[1,2-a]pyridine-8-carboxylate (575 mg, 37.310%). MS: M/e 207 (M+1)⁺.

### Step 2: methyl 6-(bromomethyl)imidazo[1,2-a]pyridine-8-carboxylate

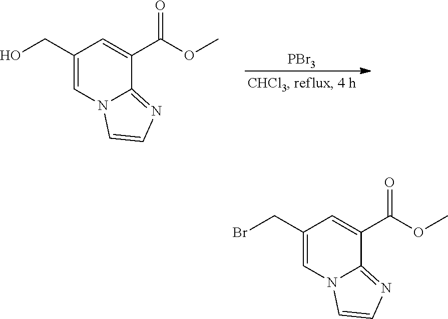

To a stirred solution of methyl 6-(hydroxymethyl)imidazo[1,2-a]pyridine-8-carboxylate (550 mg, 2.534 mmol, 1 equiv, 95.0%) in CHCl₃ (3 mL, 99%) were added PBr₃ (722.00 mg, 2.534 mmol, 1 equiv, 95%) dropwise at 0° C. The resulting mixture was stirred for 3 h at 70° C. under nitrogen atmosphere. The mixture was allowed to cool down to room temperature. The resulting mixture was concentrated under vacuum. The residue was purified by silica gel column chromatography, eluted with CH₂Cl₂/MeOH (8:1) to afford methyl 6-(bromomethyl)imidazo[1,2-a]pyridine-8-carboxylate (410 mg, 57.18%). MS: M/e 269 (M+1)⁺.

### Step 3: methyl 6-((isobutylamino)methyl)imidazo[1,2-a]pyridine-8-carboxylate

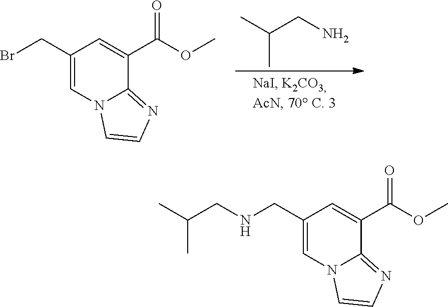

To a stirred solution of methyl 6-(bromomethyl)imidazo[1,2-a]pyridine-8-carboxylate (65 mg, 0.229 mmol, 1 equiv, 95%) and isobutylamine (35.33 mg, 0.458 mmol, 2 equiv, 95%) in acetonitrile were added K₂CO₃ (66.77 mg, 0.458 mmol, 2 equiv, 95%) and NaI (36.21 mg, 0.229 mmol, 1 equiv, 95%) at room temperature under nitrogen atmosphere. The resulting mixture was stirred for additional 3 h at 70° C. The resulting mixture was concentrated under reduced pressure. The residue was purified by silica gel column chromatography, eluted with CH₂Cl₂/MeOH (10:1) to afford methyl 6-([(2-methylpropyl)amino]methylimidazo[1,2-a]pyridine-8-carboxylate (50 mg, 75.04%). MS: M/e 262 (M+1)⁺.

### Step 4: 6-((isobutylamino)methyl)imidazo[1,2-a]pyridine-8-carboxylic acid

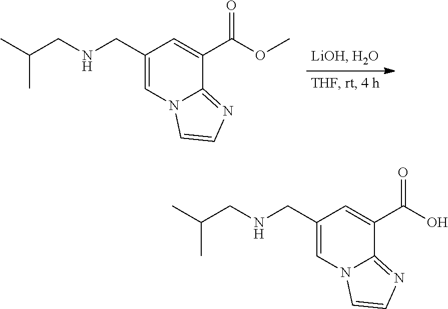

A solution of methyl 6-([(2-methylpropyl)amino]methylimidazo[1,2-a]pyridine-8-carboxylate (45 mg, 0.164 mmol, 1 equiv, 95%) and LiOH (16.50 mg, 0.656 mmol, 4 equiv, 95%) in THF/H₂O was stirred for 2 h at room temperature under nitrogen atmosphere. The mixture was acidified to pH 2 with HCl (aq.). The residue was purified by reversed-phase flash chromatography with the following conditions: column, C18 silica gel; mobile phase, MeCN in Water (0.1% TFA), 0% to 100% gradient in 30 min; detector, UV 254 nm. This resulted in 6-([(2-methylpropyl)amino]methylimidazo[1,2-a]pyridine-8-carboxylic acid (35 mg, 82.19%). MS: M/e 248 (M+1)⁺.

### Step 5: 6-((isobutylamino)methyl)-N-(5-((1s, 3s)-3-methyl-1-(4-methyl-4H-1,2,4-triazol-3-yl)cyclobutyl)pyridin-3-yl)imidazo[1,2-a]pyridine-8-carboxamide

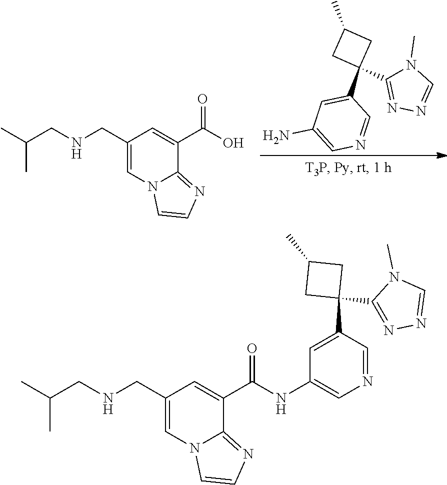 [SMILES: CC(C)CNCc1cc(C(=O)O)c2nccn2c1 ; Cn1cnnc1[C@]1(c2cncc(N)c2)C[C@H](C)C1 ; CC(C)CNCc1cc(C(=O)Nc2cncc([C@]3(c4nncn4C)C[C@@H](C)C3)c2)c2nccn2c1]

To a stirred solution of 6-([(2-methylpropyl)amino]methylimidazo[1,2-a]pyridine-8-carboxylic acid (30 mg, 0.115 mmol, 1 equiv, 95%) and 5-[(1r, 3s)-3-methyl-1-(4-methyl-1,2,4-triazol-3-yl)cyclobutyl]pyridin-3-amine (35.42 mg, 0.138 mmol, 1.2 equiv, 95%) in pyridine was added tripropyl-1,3,5,2lambda5,4lambda5,6lambda5-trioxatriphosphinane-2,4,6-trione (733.38 mg, 1.150 mmol, 10 equiv, 50%) at 0° C. under nitrogen atmosphere. The resulting mixture was stirred for additional 1 h at room temperature. The crude product was purified by Chiral-Prep-HPLC with the following conditions (2 #SHIMADZU (HPLC-01)): Column: XBridge Prep OBD C18 Column, 30*150 mm, 5 μm; mobile phase, 10 mmol NH4HCO3+0.05% NH3H2O and ACN (25% ACN up to 55% in 8 min); This resulted in 6-([(2-methylpropyl)amino]methyl-N-(5-[(1r, 3s)-3-methyl-1-(4-methyl-1,2,4-triazol-3-yl)cyclobutyl]pyridin-3-ylimidazo[1,2-a]pyridine-8-carboxamide (19.6 mg, 34.37%). MS: M/e 473 (M+1)⁺. ¹H NMR (400 MHz, DMSO-d₆) δ 12.52 (s, 1H), 8.86 (m, 1H), 8.75 (m, 1H), 8.41 (m, 1H), 8.34 (s, 1H), 8.16 (m, 2H), 8.13 (m, 1H), 7.80 (m, 1H), 3.77 (s, 2H), 3.24 (s, 3H), 2.91 (m, 2H), 2.59 (m, 3H), 2.32 (m, 2H), 1.69 (m, 1H), 1.10 (m, 3H), 0.88 (m, 6H).

### Compound A79: 3-fluoro-6-((isobutylamino)methyl)-N-(5-((1s, 3s)-3-methyl-1-(4-methyl-4H-1,2,4-triazol-3-yl)cyclobutyl)pyridin-3-yl)imidazo[1,2-a]pyridine-8-carboxamide

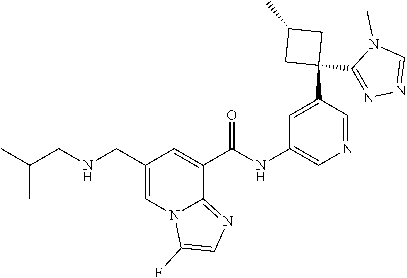 [SMILES: CC(C)CNCc1cc(C(=O)Nc2cncc([C@]3(c4nncn4C)C[C@@H](C)C3)c2)c2ncc(F)n2c1]

### Step 1: methyl 3-fluoro-6-((isobutylamino)methyl)imidazo[1,2-a]pyridine-8-carboxylate

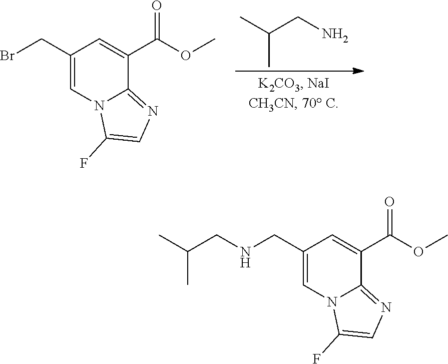

To a stirred solution of methyl 6-(bromomethyl)-3-fluoroimidazo[1,2-a]pyridine-8-carboxylate (160 mg, 0.529 mmol, 1 equiv, 95%) and isobutylamine (81.52 mg, 1.058 mmol, 2 equiv, 95%) in acetonitrile were added K2CO3 (154.05 mg, 1.058 mmol, 2 equiv, 95%) and NaI (83.54 mg, 0.529 mmol, 1 equiv, 95%) at room temperature under nitrogen atmosphere. The resulting mixture was stirred for additional 3 h at 70° C. The resulting mixture was concentrated under vacuum. The residue was purified by silica gel column chromatography, eluted with CH2Cl2/MeOH (10:1) to afford methyl 3-fluoro-6-([(2-methylpropyl)amino]methylimidazo[1,2-a]pyridine-8-carboxylate (75 mg, 45.64%). MS: M/e 280 (M+1)⁺.

### Step 2: 3-fluoro-6-((isobutylamino)methyl)imidazo[1,2-a]pyridine-8-carboxylic acid

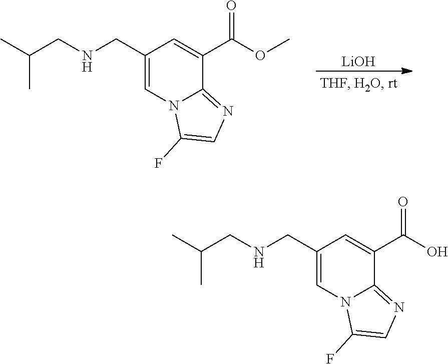

A solution of methyl 3-fluoro-6-([(2-methylpropyl)amino]methylimidazo[1,2-a]pyridine-8-carboxylate (65 mg, 0.221 mmol, 1 equiv, 95%) and LiOH (22.29 mg, 0.884 mmol, 4 equiv, 95%) in THF/H2O was stirred for 2 h at room temperature under nitrogen atmosphere. The mixture was acidified to pH 2 with HCl (aq.). The residue was purified by reversed-phase flash chromatography with the following conditions: column, C18 silica gel; mobile phase, MeOH in Water (0.1% TFA), 0% to 100% gradient in 30 min; detector, UV 254 nm. This resulted in 3-fluoro-6-([(2-methylpropyl)amino]methylimidazo[1,2-a]pyridine-8-carboxylic acid (55 mg, 84.40%). MS: M/e 266 (M+1)⁺.

### Step 3: 3-fluoro-6-((isobutylamino)methyl)-N-(5-((1s, 3s)-3-methyl-1-(4-methyl-4H-1,2,4-triazol-3-yl)cyclobutyl)pyridin-3-yl)imidazo[1,2-a]pyridine-8-carboxamide

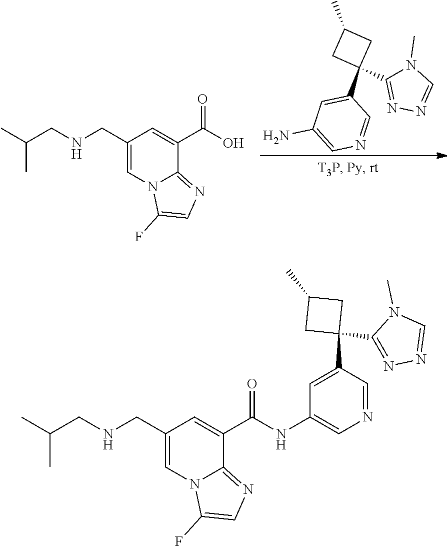 [SMILES: CC(C)CNCc1cc(C(=O)O)c2ncc(F)n2c1 ; Cn1cnnc1[C@]1(c2cncc(N)c2)C[C@H](C)C1 ; CC(C)CNCc1cc(C(=O)Nc2cncc([C@]3(c4nncn4C)C[C@@H](C)C3)c2)c2ncc(F)n2c1]

To a stirred solution of 3-fluoro-6-([(2-methylpropyl)amino]methylimidazo[1,2-a]pyridine-8-carboxylic acid (50 mg, 0.179 mmol, 1 equiv, 95%) and 5-[(1r, 3s)-3-methyl-1-(4-methyl-1,2,4-triazol-3-yl)cyclobutyl]pyridin-3-amine (55.03 mg, 0.215 mmol, 1.2 equiv, 95%) in pyridine was added T3P (1139.41 mg, 1.790 mmol, 10 equiv, 50%) dropwise at 0° C. under nitrogen atmosphere. The resulting mixture was stirred for additional 1 h at room temperature. The crude product was purified by Prep-HPLC with the following conditions (2 #SHIMADZU (HPLC-01)): Column: XBridge Prep OBD C18 Column, 30*150 mm, 5 μm; mobile phase, 10 mmol NH4HCO3+0.05% NH3H2O and ACN (15% ACN up to 45% in 8 min). This resulted in 3-fluoro-6-([(2-methylpropyl)amino]methyl-N-(5-[(1r, 3s)-3-methyl-1-(4-methyl-1,2,4-triazol-3-yl)cyclobutyl]pyridin-3-ylimidazo[1,2-a]pyridine-8-carboxamide (22.0 mg, 24.32%). MS: M/e 491 (M+1)⁺. ¹H NMR (400 MHz, DMSO-d₆) δ 12.02 (s, 1H), 8.85 (m, 1H), 8.49 (m, 1H), 8.42 (m, 1H), 8.34 (s, 1H), 8.18 (m, 1H), 8.11 (m, 1H), 7.63 (m, 1H), 3.80 (s, 2H), 3.24 (s, 3H), 2.91 (m, 2H), 2.59 (m, 3H), 2.32 (m, 2H), 1.68 (m, 1H), 1.10 (m, 3H), 0.87 (m, 6H) ppm.

### Compound A80: 3-methyl-N-(5-((1s, 3s)-3-methyl-1-(4-methyl-4H-1,2,4-triazol-3-yl)cyclobutyl)pyridin-3-yl)-6-(((1-methylcyclobutyl)amino)methyl)imidazo[1,2-a]pyridine-8-carboxamide

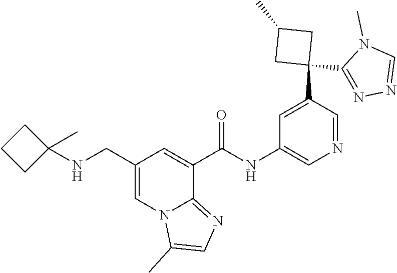 [SMILES: Cc1cnc2c(C(=O)Nc3cncc([C@]4(c5nncn5C)C[C@@H](C)C4)c3)cc(CNC3(C)CCC3)cn12]

### Step 1: methyl 3-methyl-6-(((1-methylcyclobutyl)amino)methyl)imidazo[1,2-a]pyridine-8-carboxylate

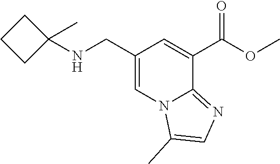 [SMILES: COC(=O)c1cc(CNC2(C)CCC2)cn2c(C)cnc12]

To a solution of 1-methylcyclobutan-1-amine hydrochloride (83.60 mg, 687.42 mol) in MeOH (2 mL) was added TEA (69.56 mg, 687.42 mol), methyl 6-formyl-3-methylimidazo[1,2-a]pyridine-8-carboxylate (200 mg, 458.28 μmol) and CH3COOH (27.52 mg, 458.28 μmol). The mixture was stirred at 20° C. for 2 hrs. Then NaBH3CN (57.60 mg, 916.56 mol) was added at 20° C. The resulting mixture was stirred at 20° C. for 14 hr. The mixture was adjusted to pH 10 with saturated aqueous Na2CO3, and then extracted with EtOAc 30 mL (10 mL*3). The combined organic layers were washed with brine 30 mL (10 mL*3), dried over Na2SO4, filtered and concentrated under reduced pressure to give a residue, which was purified by prep-HPLC (column: Waters Xbridge C18 150*50 mm*10 um; mobile phase: [H2O (10 mM NH4HCO3)-ACN]; gradient: 20%-50% B over 8.0 min) to give the product (40 mg, 30.37% yield). MS: M/e 288 (M+1); 1H NMR (400 MHz, DMSO-d₆) δ=8.40 (s, 1H), 7.87 (d, J=1.4 Hz, 1H), 7.41 (s, 1H), 3.89 (s, 3H), 3.69 (s, 2H), 2.47 (s, 3H), 2.05-1.94 (m, 2H), 1.78-1.65 (m, 4H), 1.25 (s, 3H) ppm.

### Step 2: lithium 3-methyl-6-(((1-methylcyclobutyl)amino)methyl)imidazo[1,2-a]pyridine-8-carboxylate

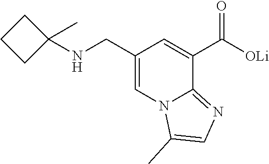

A mixture of methyl 3-methyl-6-(((1-methylcyclobutyl)amino)methyl)imidazo[1,2-a]pyridine-8-carboxylate (40 mg, 139.20 μmol), LiOH·H2O (7.01 mg, 167.04 μmol) in H2O (1 mL), MeOH (1 mL), THF (1 mL) was degassed and purged with N2 for 3 times, and then the mixture was stirred at 20° C. for 1 hr under N2 atmosphere. The reaction mixture was concentrated under reduced pressure to give a residue, which was used into the next step without further purification to give the product (40 mg, crude). MS: M/e 274 (M+1); ¹H NMR (400 MHz, DMSO-d₆) δ=8.18 (s, 1H), 7.88 (s, 1H), 7.28 (s, 1H), 3.67 (d, J=8.0 Hz, 2H), 2.45 (s, 3H), 2.17 (br t, J=7.6 Hz, 1H), 2.06-1.94 (m, 2H), 1.81-1.62 (m, 4H), 1.27 (s, 3H) ppm.

### Step 3: 3-methyl-N-(5-((1s, 3s)-3-methyl-1-(4-methyl-4H-1,2,4-triazol-3-yl)cyclobutyl)pyridin-3-yl)-6-(((1-methylcyclobutyl)amino)methyl)imidazo[1,2-a]pyridine-8-carboxamide

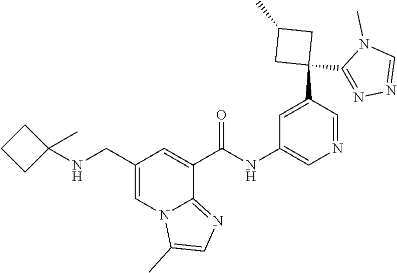 [SMILES: Cc1cnc2c(C(=O)Nc3cncc([C@]4(c5nncn5C)C[C@@H](C)C4)c3)cc(CNC3(C)CCC3)cn12]

To a stirred solution of lithium 3-methyl-6-(((1-methylcyclobutyl)amino)methyl)imidazo[1,2-a]pyridine-8-carboxylate (40 mg, 114.59 mol) and 5-((1s, 3s)-3-methyl-1-(4-methyl-4H-1,2,4-triazol-3-yl)cyclobutyl)pyridin-3-amine (27.88 mg, 114.59 mol) in PYRIDINE (2 mL) was added T3P (218.76 mg, 343.76 mol) at 20° C. The resulting mixture was stirred for 2 h at 20° C. The reaction mixture was quenched by addition H2O 10 mL at 20° C., and then extracted with EtOAc 30 mL (10 mL*3). The combined organic layers were washed with brine 30 mL (10 mL*3), dried over Na2SO4, filtered and concentrated under reduced pressure to give a residue, which was purified by prep-HPLC (column: Waters Xbridge BEH C18 100*30 mm*10 um; mobile phase: [H2O (10 mM NH4HCO3)-ACN]; gradient: 25%-55% B over 8.0 min) to give the product (6.85 mg, 11.99% yield). MS: M/e 499 (M+1)⁺; ¹H NMR (400 MHz, DMSO-d₆) δ=12.55 (s, 1H), 8.85 (d, J=2.4 Hz, 1H), 8.50 (s, 1H), 8.43-8.31 (m, 2H), 8.21-8.11 (m, 2H), 7.59 (s, 1H), 3.76 (s, 2H), 3.24 (s, 3H), 2.95-2.86 (m, 2H), 2.64-2.55 (m, 3H), 2.53 (s, 3H), 2.06-1.93 (m, 2H), 1.79-1.63 (m, 4H), 1.27 (s, 3H), 1.10 (br d, J=5.2 Hz, 3H) ppm.

### Compound A81: N-(5-((1s, 3s)-3-methyl-1-(4-methyl-4H-1,2,4-triazol-3-yl)cyclobutyl)pyridin-3-yl)-6-(((1-methylcyclobutyl)amino)methyl)imidazo[1,2-a]pyridine-8-carboxamide

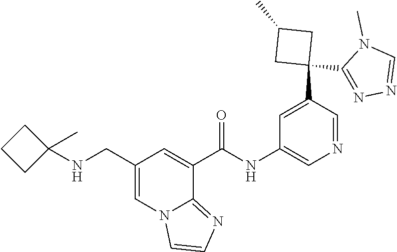 [SMILES: Cn1cnnc1[C@]1(c2cncc(NC(=O)c3cc(CNC4(C)CCC4)cn4ccnc34)c2)C[C@H](C)C1]

### Step 1: methyl 6-(((1-methylcyclobutyl)amino)methyl)imidazo[1,2-a]pyridine-8-carboxylate

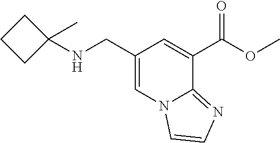 [SMILES: COC(=O)c1cc(CNC2(C)CCC2)cn2ccnc12]

To a solution of 1-methylcyclobutanamine (89.34 mg, 734.64 mol HCl) in MeOH (10 mL) was added TEA (74.34 mg, 734.64 μmol, 102.25 L), methyl 6-formylimidazo[1,2-a]pyridine-8-carboxylate (200 mg, 489.76 μmol) and acetic acid (29.41 mg, 489.76 μmol, 28.04 L) The mixture was stirred at 20° C. for 2 hr. Then NaBH₃CN (61.55 mg, 979.52 mol) was added at 20° C. The resulting mixture was stirred at 20° C. for 14 hr. The mixture was adjusted to pH 10 with saturated aqueous Na₂CO₃, and then extracted with EtOAc 30 mL (10 mL*3). The combined organic layers were washed with brine 30 mL (10 mL*3), dried over Na₂SO₄, filtered and concentrated under reduced pressure to give a residue. The crude product was purified by prep-HPLC (column: Waters Xbridge BEH C18 100*30 mm*10 um; mobile phase: [H₂O (10 mM NH4HCO₃)-ACN]; gradient: 15%-45% B over 8.0 min). Compound methyl 6-[[(1-methylcyclobutyl)amino]methyl]imidazo[1,2-a]pyridine-8-carboxylate (25.8 mg, 94.39 μmol, 19.27% yield) was obtained. MS: M/e 272 (M+1)⁺; ¹H NMR (400 MHz, DMSO-d₆) δ=8.77-8.69 (m, 1H), 8.08-7.99 (m, 1H), 7.87 (d, J=1.2 Hz, 1H), 7.67-7.52 (m, 1H), 3.89 (s, 3H), 3.65 (s, 2H), 2.02-1.90 (m, 2H), 1.78-1.60 (m, 4H), 1.35-1.17 (m, 3H) ppm.

### Step 2: lithium 6-(((1-methylcyclobutyl)amino)methyl)imidazo[1,2-a]pyridine-8-carboxylate

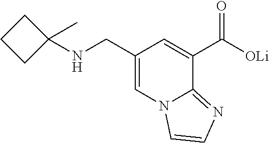

To a solution of methyl 6-[[(1-methylcyclobutyl)amino]methyl]imidazo[1,2-a]pyridine-8-carboxylate (25.8 mg, 94.39 μmol) in MeOH (1 mL) H2O (1 mL) THF (1 mL) was added LiOH·H₂O (4.75 mg, 113.27 mol). The mixture was stirred at 20° C. for 2 hr. The reaction mixture was concentrated under reduced pressure to give a residue. Without purification. Compound [6-[[(1-methylcyclobutyl)amino]methyl]imidazo[1,2-a]pyridine-8-carbonyl]oxylithium (19.8 mg, crude) was obtained. MS: M/e 260 (M+1)⁺; ¹H NMR (400 MHz, DMSO-d₆) δ=8.48 (s, 1H), 7.93 (d, J=1.2 Hz, 1H), 7.86 (s, 1H), 7.48-7.43 (m, 1H), 3.72-3.58 (m, 2H), 2.26-2.18 (m, 1H), 2.05-1.94 (m, 2H), 1.79-1.64 (m, 4H), 1.28-1.22 (m, 3H) ppm.

### Step 3: N-(5-((1s, 3s)-3-methyl-1-(4-methyl-4H-1,2,4-triazol-3-yl)cyclobutyl)pyridin-3-yl)-6-(((1-methylcyclobutyl)amino)methyl)imidazo[1,2-a]pyridine-8-carboxamide

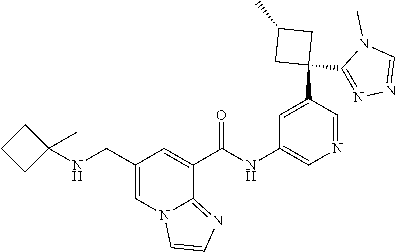 [SMILES: Cn1cnnc1[C@]1(c2cncc(NC(=O)c3cc(CNC4(C)CCC4)cn4ccnc34)c2)C[C@H](C)C1]

To a stirred solution of lithium 6-(((1-methylcyclobutyl)amino)methyl)imidazo[1,2-a]pyridine-8-carboxylate (19.8 mg, 59.72 mol) and 5-[3-methyl-1-(4-methyl-1,2,4-triazol-3-yl)cyclobutyl]pyridin-3-amine (14.53 mg, 59.72 mol) in pyridine (1 mL) was added T₃P (114.01 mg, 179.16 mol) at 20° C. The resulting mixture was stirred for 2 h at 20° C. The reaction mixture was quenched by addition H₂O 10 mL at 20° C., and then extracted with EtOAc 30 mL (10 mL*3). The combined organic layers were washed with brine 30 mL (10 mL*3), dried over Na₂SO₄, filtered and concentrated under reduced pressure to give a residue. The crude product was purified by prep-HPLC (column: Waters Xbridge BEH C18 100*30 mm*10 um; mobile phase: [H2O (10 mM NH4HCO3)-ACN]; gradient: 20%-50% B over 8.0 min) to give the product (4.24 mg, 14.65% yield). MS: M/e 485 (M+1), ¹H NMR (400 MHz, DMSO-d₆) δ=12.51 (s, 1H), 8.86 (d, J=2.12 Hz, 1H), 8.78 (s, 1H), 8.41 (d, J=1.92 Hz, 1H), 8.34 (s, 1H), 8.20-8.09 (m, 3H), 7.79 (d, J=1.12 Hz, 1H), 3.72 (s, 2H), 3.24 (s, 3H), 2.95-2.86 (m, 2H), 2.59 (d, J=6.8 Hz, 3H), 2.04-1.94 (m, 2H), 1.78-1.65 (m, 4H), 1.26 (s, 3H), 1.10 (d, J=5.32 Hz, 3H) ppm.

### Compound A82: 3-chloro-N-(5-((1s, 3s)-3-methyl-1-(4-methyl-4H-1,2,4-triazol-3-yl)cyclobutyl)pyridin-3-yl)-6-(((1-methylcyclobutyl)amino)methyl)imidazo[1,2-a]pyridine-8-carboxamide

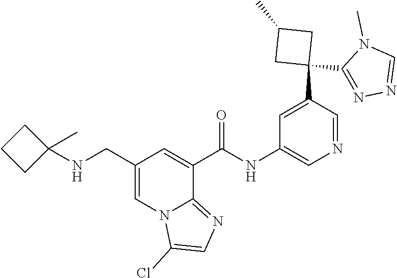 [SMILES: Cn1cnnc1[C@]1(c2cncc(NC(=O)c3cc(CNC4(C)CCC4)cn4c(Cl)cnc34)c2)C[C@H](C)C1]

### Step 1: methyl 3-chloro-6-(((1-methylcyclobutyl)amino)methyl)imidazo[1,2-a]pyridine-8-carboxylate

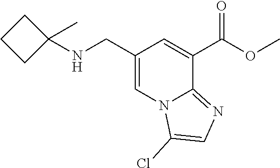 [SMILES: COC(=O)c1cc(CNC2(C)CCC2)cn2c(Cl)cnc12]

To a solution of 1-methylcyclobutanamine (110.08 mg, 905.18 mol) in MeOH (10 mL) was added TEA (91.60 mg, 905.18 mol), methyl 3-chloro-6-formyl-imidazo[1,2-a]pyridine-8-carboxylate (200 mg, 603.45 mol) and acetic acid (36.24 mg, 603.45 mol) The mixture was stirred at 20° C. for 2 hrs. Then NaBH₃CN (75.84 mg, 1.21 mmol) was added at 20° C. The resulting mixture was stirred at 20° C. for 16 hr. The reaction mixture was added Na₂SO₄ adjust to pH 10-11 at 25° C., The aqueous phase was extracted with EA 40 mL (10 mL×4). The mixture was filtered and concentrated at 35° C. under reduced pressure to give crude product. The resulting residue was purified by prep-HPLC(column: Waters Xbridge C18 150*50 mm*10 um; mobile phase: [H₂O (10 mM NH4HCO₃)-ACN]; gradient: 25%-55% B over 8.0 min) to give the product (0.0455 g, 24.50% yield). MS: M/e 308 (M+1)⁺; 1H NMR (400 MHz, DMSO-d₆) δ=8.51 (s, 1H), 7.99 (d, J=1.6 Hz, 1H), 7.76 (s, 1H), 3.91 (s, 3H), 3.74 (s, 2H), 2.11-1.88 (m, 2H), 1.81-1.60 (m, 4H), 1.25 (s, 3H) ppm.

### Step 2: lithium 3-chloro-6-(((1-methylcyclobutyl)amino)methyl)imidazo[1,2-a]pyridine-8-carboxylate

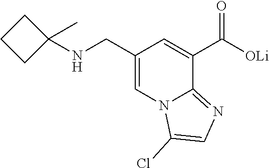

A mixture of methyl 3-chloro-6-(((1-methylcyclobutyl)amino)methyl)imidazo[1,2-a]pyridine-8-carboxylate (45.5 mg, 147.84 mol,), LiOH·H₂O (7.44 mg, 177.40 mol) in THF (1 mL) and MeOH (1 mL) and H2O (1 mL) was degassed and purged with N₂ for 3 times, and then the mixture was stirred at 25° C. for 1 hr under N₂ atmosphere. The reaction mixture was concentrated under reduced pressure to give a residue. The crude product was used into the next step without further purification to give the product (0.028 g, 63.20% yield). MS: M/e 294 (M+1)⁺.

### Step 3: 3-chloro-N-(5-((1s, 3s)-3-methyl-1-(4-methyl-4H-1,2,4-triazol-3-yl)cyclobutyl)pyridin-3-yl)-6-(((1-methylcyclobutyl)amino)methyl)imidazo[1,2-a]pyridine-8-carboxamide

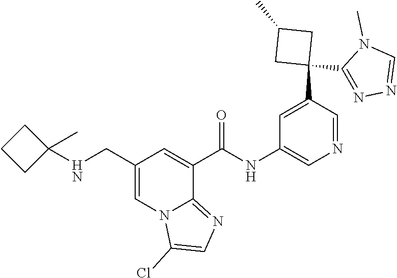 [SMILES: Cn1cnnc1[C@]1(c2cncc(NC(=O)c3cc(CNC4(C)CCC4)cn4c(Cl)cnc34)c2)C[C@H](C)C1]

To a stirred solution of lithium 3-chloro-6-(((1-methylcyclobutyl)amino)methyl)imidazo[1,2-a]pyridine-8-carboxylate (28 mg, 74.75 mol,) and 5-[3-methyl-1-(4-methyl-1,2,4-triazol-3-yl)cyclobutyl]pyridin-3-amine (18.19 mg, 74.75 mol,) in pyridine (1 mL) was added T₃P (142.70 mg, 224.24 mol, 133.49 L) at 20° C. The resulting mixture was stirred for 3 h at 20° C. The reaction mixture was quenched by addition H₂O 10 mL at 20° C., and then extracted with EtOAc 30 mL (10 mL*3). The combined organic layers were washed with brine 30 mL (10 mL*3), dried over Na₂SO₄, filtered and concentrated under reduced pressure to give a residue. The residue was purified by prep-HPLC (column: Waters Xbridge BEH C18 100*30 mm*10 um; mobile phase: [H₂O (10 mM NH4HCO₃)-ACN]; gradient: 30%-60% B over 8.0 min) to give product (0.00294 g, 7.58% yield). MS: M/e 519 (M+1)⁺; ¹H NMR (400 MHz, DMSO-d₆) δ=12.02 (s, 1H), 8.86 (d, J=2.4 Hz, 1H), 8.56 (s, 1H), 8.41 (d, J=1.92 Hz, 1H), 8.33 (s, 1H), 8.24 (d, J=1.32 Hz, 1H), 8.11 (t, J=2.0 Hz, 1H), 7.95 (s, 1H), 3.79 (s, 2H), 3.23 (s, 3H), 2.98-2.83 (m, 2H), 2.64-2.54 (m, 3H), 2.07-1.93 (m, 2H), 1.80-1.62 (m, 4H), 1.26 (s, 3H), 1.10 (d, J=5.12 Hz, 3H) ppm.

### Compound A83: 3-chloro-6-((isobutylamino)methyl)-N-(5-((1s, 3s)-3-methyl-1-(4-methyl-4H-1,2,4-triazol-3-yl)cyclobutyl)pyridin-3-yl)imidazo[1,2-a]pyridine-8-carboxamide

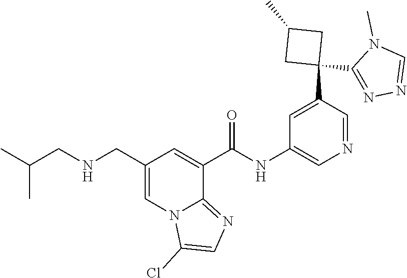 [SMILES: CC(C)CNCc1cc(C(=O)Nc2cncc([C@]3(c4nncn4C)C[C@@H](C)C3)c2)c2ncc(Cl)n2c1]

### Step 1: methyl 3-chloro-6-((isobutylamino)methyl)imidazo[1,2-a]pyridine-8-carboxylate

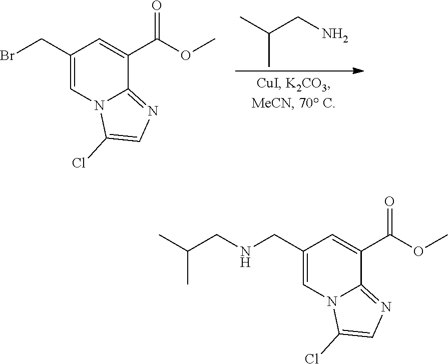

To a stirred mixture of methyl 6-(bromomethyl)-3-chloroimidazo[1,2-a]pyridine-8-carboxylate (100 mg, 0.326 mmol, 1.0 equiv, 98.9%) and isobutylamine (50 mg, 0.649 mmol, 2.0 equiv, 95%) in MeCN (2 mL) were added NaI (51 mg, 0.323 mmol, 1.0 equiv, 95%) and K₂CO₃ (95 mg, 0.653 mmol, 2.0 equiv, 95%) in portions at room temperature. The resulting mixture was stirred for 2 h at 70° C. under nitrogen atmosphere. The resulting mixture was concentrated under reduced pressure. The residue was purified by silica gel column chromatography, eluted with CH₂Cl₂/MeOH (10:1) to afford methyl 3-chloro-6-((isobutylamino)methyl)imidazo[1,2-a]pyridine-8-carboxylate (75 mg, 62.26%). MS: M/e 296 (M+1)⁺.

### Step 2: 3-chloro-6-((isobutylamino)methyl)imidazo[1,2-a]pyridine-8-carboxylic acid

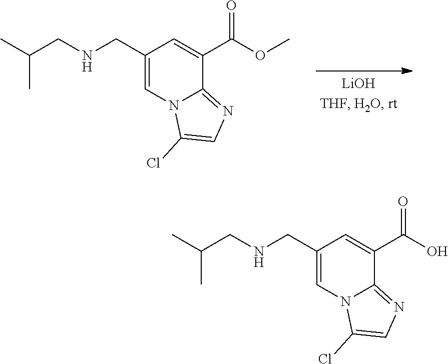

To a stirred solution of methyl 3-chloro-6-([(2-methylpropyl)amino]methylimidazo[1,2-a]pyridine-8-carboxylate (75 mg, 0.203 mmol, 1.0 equiv, 80%) in THF (1.5 mL, 95%) and H₂O (0.5 mL, 95%) were added LiOH (11 mg, 0.436 mmol, 2.0 equiv, 95%) in portions at 0° C. The resulting mixture was stirred for 1 h at room temperature under nitrogen atmosphere. The mixture was acidified to pH=5 with HCl (aq.). The resulting mixture was concentrated under vacuum. The resulting mixture was used in the next step directly without further purification. MS: M/e 282 (M+1)⁺.

### Step 3: 3-chloro-6-((isobutylamino)methyl)-N-(5-((1s, 3s)-3-methyl-1-(4-methyl-4H-1,2,4-triazol-3-yl)cyclobutyl)pyridin-3-yl)imidazo[1,2-a]pyridine-8-carboxamide

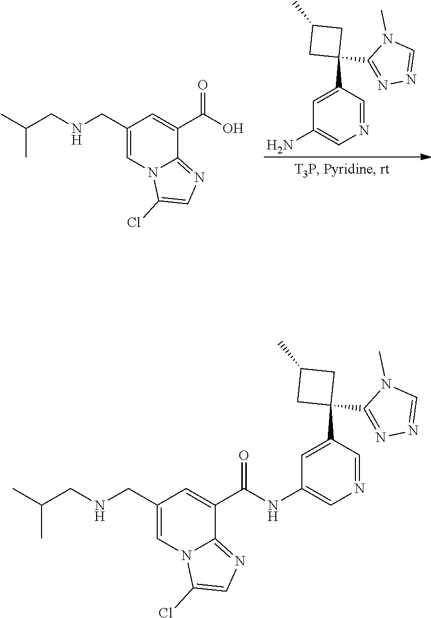 [SMILES: CC(C)CNCc1cc(C(=O)O)c2ncc(Cl)n2c1 ; Cn1cnnc1[C@]1(c2cncc(N)c2)C[C@H](C)C1 ; CC(C)CNCc1cc(C(=O)Nc2cncc([C@]3(c4nncn4C)C[C@@H](C)C3)c2)c2ncc(Cl)n2c1]

To a stirred mixture of 3-chloro-6-([(2-methylpropyl)amino]methylimidazo[1,2-a]pyridine-8-carboxylic acid (60 mg, 0.170 mmol, 2.0 equiv, 80%) and 5-[(1r, 3s)-3-methyl-1-(4-methyl-1,2,4-triazol-3-yl)cyclobutyl]pyridin-3-amine (22 mg, 0.086 mmol, 1.0 equiv, 95%) in Pyridine (1.5 mL) was added T3P (288 mg, 0.860 mmol, 10.0 equiv, 95%) dropwise at room temperature. The resulting mixture was stirred for 30 min at room temperature under nitrogen atmosphere. The resulting mixture was concentrated under reduced pressure. The crude product was purified by Prep-HPLC with the following conditions (Column: XBridge Prep OBD C18 Column, 30*150 mm, 5 μm; Mobile Phase A: Water (10 mmol/L NH₄HCO₃+0.05% NH₃·H₂O), Mobile Phase B: ACN; Flow rate: 60 mL/min mL/min; Gradient: 32% B to 62% B in 8 min; Wave Length: 254 nm; RT1 (min): 7) to afford 3-chloro-6-((isobutylamino)methyl)-N-(5-((1s, 3s)-3-methyl-1-(4-methyl-4H-1,2,4-triazol-3-yl)cyclobutyl)pyridin-3-yl)imidazo[1,2-a]pyridine-8-carboxamide (3.5 mg, 7.96%). MS: M/e 507 (M+1)⁺. ¹H NMR (300 MHz, DMSO-d₆) δ 12.04 (s, 1H), 8.86 (m=2.3 Hz, 1H), 8.55 (m=1.5 Hz, 1H), 8.42 (m=2.1 Hz, 1H), 8.34 (s, 1H), 8.25 (m=1.6 Hz, 1H), 8.12 (m=2.2 Hz, 1H), 7.96 (s, 1H), 3.85 (s, 2H), 3.24 (s, 3H), 2.91 (s, 2H), 2.59 (m=6.7 Hz, 3H), 2.33 (m=6.7 Hz, 2H), 1.69 (m=13.1, 6.6 Hz, 1H), 1.10 (m=4.8 Hz, 3H), 0.88 (m=6.6 Hz, 6H) ppm.

### Compound A84: N-(3-((1s, 3s)-3-(cyanomethyl)-1-(4-methyl-4H-1,2,4-triazol-3-yl)cyclobutyl)phenyl)-2-fluoro-6-(((1-methylcyclopropyl)amino)methyl)imidazo[1,2-a]pyridine-8-carboxamide

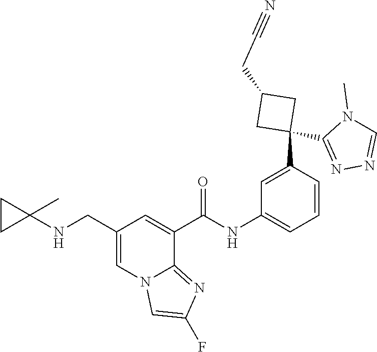 [SMILES: Cn1cnnc1[C@]1(c2cccc(NC(=O)c3cc(CNC4(C)CC4)cn4cc(F)nc34)c2)C[C@H](CC#N)C1]

### Step 1: 2-fluoro-6-(((1-methylcyclopropyl)amino)methyl)imidazo[1,2-a]pyridine-8-carboxylic acid

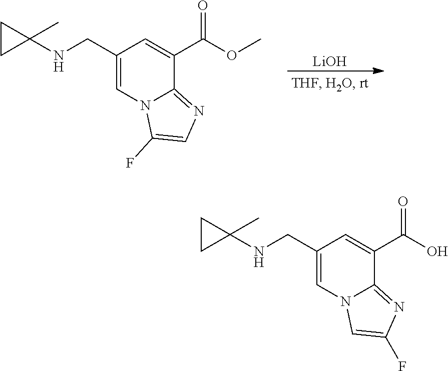 [SMILES: COC(=O)c1cc(CNC2(C)CC2)cn2c(F)cnc12 ; "LiOH" ; CC1(NCc2cc(C(=O)O)c3nc(F)cn3c2)CC1]

To a stirred solution of methyl 3-fluoro-6-(((1-methylcyclopropyl)amino)methyl)imidazo[1,2-a]pyridine-8-carboxylate (5 g, 11.594 mmol, 1 equiv, 64.3%) in THF (15 mL, 99%) and H₂O (5 mL, 99%) were added LiOH (585 mg, 23.205 mmol, 2.00 equiv, 95%) in portions. The resulting mixture was stirred for 1 h at room temperature. The mixture was acidified to pH 6 with HCl (1 M). The resulting mixture was concentrated under vacuum. The residue was purified by reversed-phase flash chromatography with the following conditions: column, C18 silica gel; mobile phase, MeCN in Water (0.1% HCl), 0%-95% in 30 min. This resulted in 2-fluoro-6-([(1-methylcyclopropyl)amino]methylimidazo[1,2-a]pyridine-8-carboxylic acid (300 mg, 9.11%). MS: M/e 264 (M+1)⁺.

### Step 2: N-(3-((1s, 3s)-3-(cyanomethyl)-1-(4-methyl-4H-1,2,4-triazol-3-yl)cyclobutyl)phenyl)-2-fluoro-6-(((1-methylcyclopropyl)amino)methyl)imidazo[1,2-a]pyridine-8-carboxamide

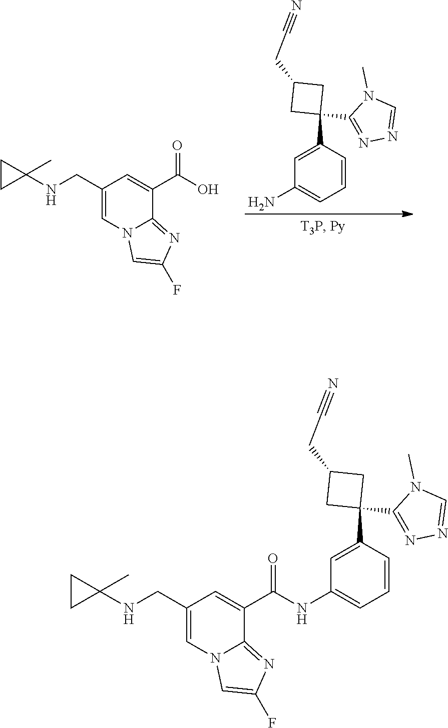 [SMILES: CC1(NCc2cc(C(=O)O)c3nc(F)cn3c2)CC1 ; Cn1cnnc1[C@]1(c2cccc(N)c2)C[C@H](CC#N)C1 ; Cn1cnnc1[C@]1(c2cccc(NC(=O)c3cc(CNC4(C)CC4)cn4cc(F)nc34)c2)C[C@H](CC#N)C1]

To a stirred mixture of 2-fluoro-6-([(1-methylcyclopropyl)amino]methylimidazo[1,2-a]pyridine-8-carboxylic acid (90 mg, 0.317 mmol, 1 equiv, 92.7%) and 2-[(1s, 3s)-3-(3-aminophenyl)-3-(4-methyl-1,2,4-triazol-3-yl)cyclobutyl]acetonitrile (87 mg, 0.320 mmol, 1.01 equiv, 98.3%) in Pyridine (6 mL, 95%) was added T₃P (1.7 g, 2.671 mmol, 8.43 equiv, 50%) dropwise at room temperature. The resulting mixture was stirred for 2 h at room temperature. The resulting mixture was concentrated under vacuum. The crude product (500 mg) was purified by Prep-HPLC with the following conditions (2 #SHIMADZU (HPLC-01)): Column, YMC-Actus Triart C18 ExRS30*150 mm, 5 m; mobile phase, 10 mmol NH₄HCO₃+0.05% NH₃H₂O and ACN (35% ACN up to 65% in 9 min); This resulted in 2-fluoro-6-([(1-methylcyclopropyl)amino]methyl-N-(3-[(1 s, 3s)-3-(cyanomethyl)-1-(4-methyl-1,2,4-triazol-3-yl)cyclobutyl]phenylimidazo[1,2-a]pyridine-8-carboxamide (76.1 mg, 46.01%). MS: M/e 513 (M+1)⁺. ¹H NMR (300 MHz, DMSO-d₆) δ 11.28 (s, 1H), 8.71-8.64 (m, 1H), 8.32 (s, 1H), 8.15-8.08 (m, 1H), 7.93 (d, J=7.6 Hz, 1H), 7.79-7.72 (m, 1H), 7.68-7.59 (m, 1H), 7.49-7.38 (m, 1H), 7.23-7.15 (m, 1H), 3.83 (s, 2H), 3.22 (s, 3H), 2.94-2.66 (m, 8H), 1.27 (s, 3H), 0.57-0.47 (m, 2H), 0.38-0.28 (m, 2H) ppm.

### Compound A85: N-(3-((1s, 3s)-1-(4-ethyl-4H-1,2,4-triazol-3-yl)-3-methylcyclobutyl)phenyl)-6-(((1-methylcyclopropyl)amino)methyl)imidazo[1,2-a]pyridine-8-carboxamide

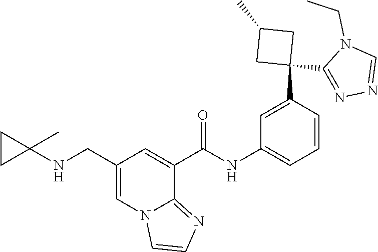 [SMILES: CCn1cnnc1[C@]1(c2cccc(NC(=O)c3cc(CNC4(C)CC4)cn4ccnc34)c2)C[C@H](C)C1]

### Step 1: 5-(1-(3-bromophenyl)-3-methylcyclobutyl)-4-ethyl-4H-1,2,4-triazole-3-thiol

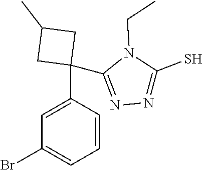 [SMILES: CCn1c(S)nnc1C1(c2cccc(Br)c2)CC(C)C1]

A mixture of 1-(3-bromophenyl)-3-methylcyclobutane-1-carbohydrazide (2.6 g, 9.18 mmol) and S═C═NCC (960.18 mg, 11.02 mmol) in THF (30 mL), the mixture was stirred at 65° C. for 1.5 h, then the mixture was added KOH (51.52 g, 91.82 mmol, 10% purity), the mixture was stirred at 65° C. for 12 hr under N₂ atmosphere. The mixture was poured into water (80 ml) and extracted with the DCM (200 ml, 100 ml). The combined organic phase was washed with brine (40 ml) and concentrated to give the residue. The residue was purified by column chromatography (SiO₂, Petroleum ether/Ethyl acetate=100/1 to 10/1) to give product (2.3 g, 71.10% yield) was obtained. MS: M/e 354 (M+1)⁺;

### Step 4: 3-(1-(3-bromophenyl)-3-methylcyclobutyl)-4-ethyl-4H-1,2,4-triazole

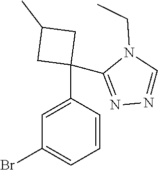 [SMILES: CCn1cnnc1C1(c2cccc(Br)c2)CC(C)C1]

To a solution of 5-(1-(3-bromophenyl)-3-methylcyclobutyl)-4-ethyl-4H-1,2,4-triazole-3-thiol (2.5 g, 7.10 mmol) in DCM (50 mL) and AcOH (5 mL) was added H₂O₂(2.41 g, 21.29 mmol, 2.05 mL, 30% purity) at 25° C. The mixture was poured into water (100 ml) and adjusted to pH 8.0 by NaOH (1 M) and extracted with DCM (200 ml, 100 ml). The combined organic phase was washed with Na₂SO₃ (100 ml) and concentrated to give the residue. The residue was purified by column chromatography (SiO₂, Petroleum ether/Ethyl acetate=100/1 to 0/1) to give product (2 g, 6.25 mmol, 88.01% yield) was obtained. MS: M/e 320 (M+1)+

### Step 5: 3-(1-(4-ethyl-4H-1,2,4-triazol-3-yl)-3-methylcyclobutyl)aniline

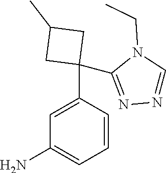 [SMILES: CCn1cnnc1C1(c2cccc(N)c2)CC(C)C1]

A mixture of 3-(1-(3-bromophenyl)-3-methylcyclobutyl)-4-ethyl-4H-1,2,4-triazole (1.5 g, 4.68 mmol), CuI (892.10 mg, 4.68 mmol), DMEDA (412.92 mg, 4.68 mmol) in NH₃·H₂O (8 mL) and SULFOLANE (4 mL) was degassed and purged with N₂ for 3 times, and then the mixture was stirred at 100° C. for 16 hr under N₂ atmosphere. The mixture was stirred at 20° C. for 12 h. The mixture was poured into water (2 ml) and extracted with DCM (10 ml, 5 ml). The mixture was concentrated. The crude product was purified by prep-HPLC(column: Waters Xbridge C18 150*50 mm*10 um; mobile phase: [H2O (10 mM NH₄HCO3)-ACN]; gradient: 20%-40% B over 8.0 min) to give the product (0.225 g, 18.74% yield). MS: M/e 257 (M+1)⁺; ¹H NMR (400 MHz, DMSO-d₆) δ=8.45 (s, 1H), 6.99 (t, J=7.9 Hz, 1H), 6.50-6.40 (m, 3H), 5.05 (s, 2H), 3.54-3.45 (m, 2H), 2.76-2.65 (m, 2H), 2.45 (q, J=8.0 Hz, 2H), 1.10-1.02 (m, 3H), 0.96-0.85 (m, 3H).

### Step 6: tert-butyl ((8-((3-((1s, 3s)-1-(4-ethyl-4H-1,2,4-triazol-3-yl)-3-methylcyclobutyl)phenyl)carbamoyl)imidazo[1,2-a]pyridin-6-yl)methyl)(1-methylcyclopropyl)carbamate

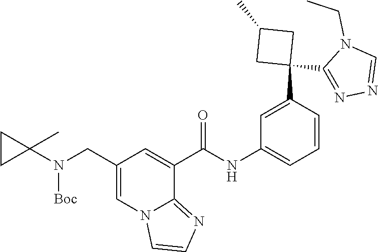 [SMILES: CCn1cnnc1[C@]1(c2cccc(NC(=O)c3cc(CN(C(=O)OC(C)(C)C)C4(C)CC4)cn4ccnc34)c2)C[C@H](C)C1]

To a solution of 3-(1-(4-ethyl-4H-1,2,4-triazol-3-yl)-3-methylcyclobutyl)aniline (50 mg, 195.05 mol) and lithium 6-(((tert-butoxycarbonyl)(1-methylcyclopropyl)amino)methyl)imidazo[1,2-a]pyridine-8-carboxylate (67.37 mg, 195.05 mol) in PYRIDINE (1 mL) was added T₃P (372.37 mg, 585.15 mol, 348.33 L, 50% purity) at 25° C. The reaction mixture was quenched by addition H₂O 10 mL at 20° C., and then extracted with EtOAc 30 mL (10 mL*3). The combined organic layers were washed with brine 30 mL (10 mL*3), dried over Na₂SO₄, filtered and concentrated under reduced pressure to give a residue The residue was purified by prep-HPLC (column: Waters Xbridge BEH C18 100*30 mm*10 um; mobile phase: [A: H2O (10 mM NH₄HCO₃); B: ACN]; B %: 45.00%-75.00%, 8.00 min) to give product (0.067 g, 114.78 mol, 58.85% yield) was obtained. MS: M/e 585 (M+1)⁺; ¹H NMR (400 MHz, DMSO-d₆) δ=12.35 (s, 1H), 8.65 (s, 1H), 8.39 (s, 1H), 8.19 (d, J=1.32 Hz, 1H), 8.05 (d, J=1.4 Hz, 1H), 7.79 (d, J=1.3 Hz, 1H), 7.80-7.74 (m, 1H), 7.74-7.60 (m, 2H), 7.40 (t, J=7.92 Hz, 1H), 7.15 (d, J=7.62 Hz, 1H), 4.46 (s, 2H), 3.51 (q, J=7.32 Hz, 2H), 2.86-2.77 (m, 2H), 1.41 (s, 8H), 1.24-1.18 (m, 1H), 1.14-1.05 (m, 6H), 0.90 (t, J=7.32 Hz, 3H), 0.82 (s, 2H), 0.67-0.52 (m, 2H);

### Step 7: N-(3-((1s, 3s)-1-(4-ethyl-4H-1,2,4-triazol-3-yl)-3-methylcyclobutyl)phenyl)-6-(((1-methylcyclopropyl)amino)methyl)imidazo[1,2-a]pyridine-8-carboxamide

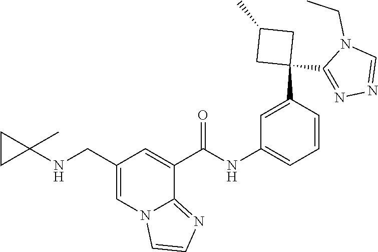 [SMILES: CCn1cnnc1[C@]1(c2cccc(NC(=O)c3cc(CNC4(C)CC4)cn4ccnc34)c2)C[C@H](C)C1]

A mixture of tert-butyl ((8-((3-((1s, 3s)-1-(4-ethyl-4H-1,2,4-triazol-3-yl)-3-methylcyclobutyl)phenyl)carbamoyl)imidazo[1,2-a]pyridin-6-yl)methyl)(1-methylcyclopropyl)carbamate (0.067 g, 114.78 mol) in DCM (2 mL) and TFA (1 mL) was degassed and purged with N₂ for 3 times, and then the mixture was stirred at 20° C. for 1 hr under N₂ atmosphere. The mixture was adjusted to pH=7.0-8.0 by NaHCO₃ and extracted with DCM (10 ml, 5 ml). The combined organic phase was concentrated to give the residue which was purified by prep-HPLC (column: Waters Xbridge BEH C18 100*30 mm*10 um; mobile phase: [H2O (10 mM NH₄HCO₃)-ACN]; gradient: 25%-65% B over 8.0 min) to give the product (35.49 mg, 63.94% yield). MS: M/e 484 (M+1)⁺; ¹H NMR (400 MHz, DMSO-d₆) δ=12.41 (s, 1H), 8.72 (s, 1H), 8.42 (s, 1H), 8.16-8.08 (m, 2H), 7.84-7.62 (m, 3H), 7.42 (t, J=7.92 Hz, 1H), 7.16 (d, J=7.8 Hz, 1H), 3.82 (s, 2H), 3.53 (q, J=7.12 Hz, 2H), 2.83 (d, J=3.0 Hz, 2H), 2.55 (d, J=6.42 Hz, 3H), 1.27 (s, 3H), 1.09 (d, J=4.92 Hz, 3H), 0.92 (t, J=7.32 Hz, 3H), 0.55-0.47 (m, 2H), 0.36-0.29 (m, 2H).

### Compound A86: 3-methyl-N-(5-((1s, 3s)-3-methyl-1-(4-methyl-4H-1,2,4-triazol-3-yl)cyclobutyl)pyridin-3-yl)-6-((((1s, 3s)-3-methylcyclobutyl)amino)methyl)imidazo[1,2-a]pyridine-8-carboxamide

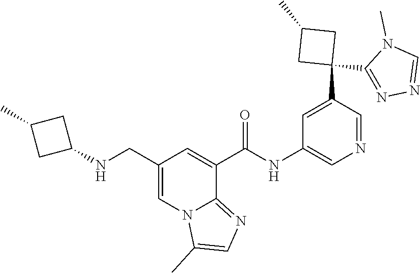 [SMILES: Cc1cnc2c(C(=O)Nc3cncc([C@]4(c5nncn5C)C[C@@H](C)C4)c3)cc(CN[C@H]3C[C@@H](C)C3)cn12]

The compound A86 was synthesized starting from the corresponding starting materials according the similar procedures described as those of Compound A76 to give the title compound (21.3 mg). ¹H NMR (400 MHz, DMSO-d₆) δ 12.54 (s, 1H), 8.85 (s, 1H), 8.46 (s, 1H), 8.40 (s, 1H), 8.34 (s, 1H), 8.15-8.13 (m, 2H), 7.58 (s, 1H), 3.73 (s, 2H), 3.24 (s, 3H), 2.99-2.91 (m, 3H), 2.58-2.48 (m, 7H), 2.35-2.27 (m, 2H), 1.92-1.83 (m, 1H), 1.33-0.99 (m, 8H) ppm. MS: M/e 499 (M+1).

### Compound A87: N-(3-((1s, 3s)-3-methyl-1-(4-methyl-4H-1,2,4-triazol-3-yl)cyclobutyl)phenyl)-6-(((1-methylcyclopropyl)amino)methyl)-[1,2,4]triazolo[1,5-a]pyridine-8-carboxamide

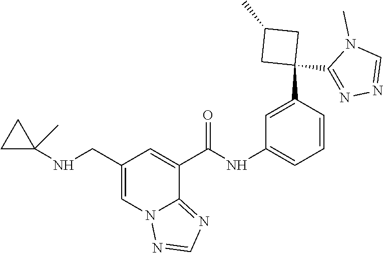 [SMILES: Cn1cnnc1[C@]1(c2cccc(NC(=O)c3cc(CNC4(C)CC4)cn4ncnc34)c2)C[C@H](C)C1]

### Step A: methyl 6-bromo-[1,2,4]triazolo[1,5-a]pyridine-8-carboxylate

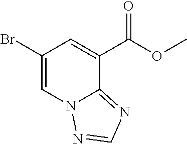

To a solution of methyl 2-amino-5-bromonicotinate (5 g, 21.64 mmol) in MeOH (70 mL) was added DMF-DMA (5.8 mL, 43.72 mmol). The reaction mixture was stirred for 12 h at 70° C. The reaction was cooled down to room temperature, and Hydroxylamine hydrochloride (3 g, 43.48 mmol) was added. The reaction mixture was stirred for 2 h at 25° C. The solvent was removed by in vacuo. The residue was dissolved in THF (50 mL) and TFAA (9 mL, 63.86 mmol) was added dropwise at 0° C. The reaction mixture was stirred for 12 h at 25° C. After completed, the reaction was quenched with aq NaHCO₃ (50 mL) and extracted with DCM/MeOH (10/1, 3×50 mL), dried over Na₂SO₄ and concentrated under vacuum. The residue was triturated with DMF (25 mL) and the mixture was filtrated and the solid was washed with MeOH (2×3 mL), drying to afford the title compound (2.4 g). The filate was collected and concentrated under vacuum. The residue was purified by Prep-HPLC to afford the title compound (2.6 g). ¹H NMR (400 MHz, DMSO-d₆) δ 9.71 (d, J=1.8 Hz, 1H), 8.65 (s, 1H), 8.31 (d, J=1.8 Hz, 1H), 3.94 (s, 3H) ppm. MS: M/e 256, 258 (M+H)⁺.

### Step B: methyl 6-vinyl-[1,2,4]triazolo[1,5-a]pyridine-8-carboxylate

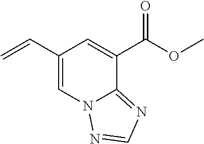

To a solution of methyl 6-bromo-[1,2,4]triazolo[1,5-a]pyridine-8-carboxylate (2.4 g, 9.37 mmol), potassium trifluoro(vinyl)borate (1.6 g, 11.94 mmol) in Dioxane (25 mL)/H₂O (2.5 mL) was added Xphos G2 Pd (370 mg, 0.47 mmol) and Cs₂CO₃ (6.1 g, 18.71 mmol) under N₂. The reaction mixture was stirred for 12 h at 85° C. After completed, the mixture was filtered and the filate was collected and concentrated under vacuum. The residue was purified by column chromatography on silica gel eluting with methanol in dichloromethane (6%) to afford the title compound (1.7 g, 89%). ¹H NMR (400 MHz, DMSO-d₆) δ 9.37 (s, 1H), 8.60 (s, 1H), 8.46 (s, 1H), 6.88 (dd, J=17.6, 11.2 Hz, 1H), 6.10 (d, J=17.6 Hz, 1H), 5.48 (d, J=11.2 Hz, 1H), 3.95 (s, 3H) ppm. MS: M/e 204 (M+H)⁺.

### Step C: methyl 6-formyl-[1,2,4]triazolo[1,5-a]pyridine-8-carboxylate

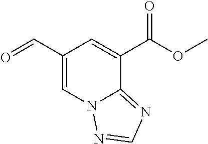

To a solution of methyl 6-vinyl-[1,2,4]triazolo[1,5-a]pyridine-8-carboxylate (1.7 g, 8.37 mmol), K₂OsO₄ 2H₂O (180 mg, 0.49 mmol) in Dioxane (60 mL)/H₂O (20 mL) was added NaIO₄ (5.4 g, 25.23 mmol). The reaction mixture was stirred for 3 h at 25° C. The reaction mixture was diluted with THF (100 mL) and filtered. The filate was collected and concentrated under vacuum. The residue was triturated with water (40 mL) (stirred for 12 h at 25° C.) and the mixture was filtrated and the solid was washed with water (2×10 mL), drying to afford the title compound (1.2 g). The filate was collected and extracted with DCM/MeOH (10/1, 3×50 mL), dried over Na₂SO₄ and concentrated under vacuum to afford the title compound (500 mg). The product (1.7 g, impure) was obtained. ¹H NMR (400 MHz, DMSO-d₆) δ 10.09 (s, 1H), 9.99 (d, J=1.2 Hz, 1H), 8.84 (s, 1H), 8.52 (d, J=1.2 Hz, 1H), 3.96 (s, 3H) ppm. MS: M/e 206 (M+H)⁺.

### Step D: methyl 6-(((1-methylcyclopropyl)amino)methyl)-[1,2,4]triazolo[1,5-a]pyridine-8-carboxylate

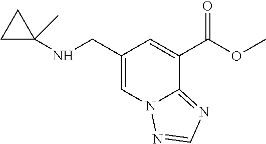 [SMILES: COC(=O)c1cc(CNC2(C)CC2)cn2ncnc12]

To a solution of methyl 6-formyl-[1,2,4]triazolo[1,5-a]pyridine-8-carboxylate (1 g, impure) and 1-methylcyclopropan-1-amine hydrochloride (790 mg, 7.31 mmol), Et₃N (740 mg, 7.32 mmol) in DCM (30 mL) was added AcOH (590 mg, 9.83 mmol) at 0° C. After stirring for 10 min, NaBH(OAc)₃ (2.1 g, 9.91 mmol) was added. The reaction mixture was stirred for 2 h at 25° C. NaBH(OAc)₃ (1 g, 4.72 mmol) was added. The reaction mixture was stirred for 14 h at 25° C. After completed, the reaction was quenched with aq NaHCO₃ to adjust PH=8-9 at 0° C. and extracted with DCM (3×50 mL). The organic phase was washed with aq NaHCO₃ (2×50 mL), brine (2×50 mL), dried over Na₂SO₄ and concentrated under vacuum. The residue was purified by column chromatography on silica gel eluting with methanol in dichloromethane (6%) to afford the title compound (940 mg, 74%). ¹H NMR (400 MHz, DMSO-d₆) δ 9.08 (s, 1H), 8.56 (s, 1H), 8.25 (s, 1H), 3.93 (s, 3H), 3.85 (s, 2H), 2.64 (s, 1H), 1.25 (s, 3H), 0.55-0.43 (m, 2H), 0.36-0.26 (m, 2H) ppm. MS: M/e 261 (M+H)⁺.

### Step E: methyl 6-(((tert-butoxycarbonyl)(1-methylcyclopropyl)amino)methyl)-[1,2,4]triazolo[1,5-a]pyridine-8-carboxylate

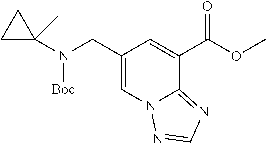 [SMILES: COC(=O)c1cc(CN(C(=O)OC(C)(C)C)C2(C)CC2)cn2ncnc12]

To a solution of methyl 6-(((1-methylcyclopropyl)amino)methyl)-[1,2,4]triazolo[1,5-a]pyridine-8-carboxylate (900 mg, 3.46 mmol), Et₃N (700 mg, 6.93 mmol) in DCM (15 mL) was added Boc₂O (1.1 g, 5.05 mmol) in DCM (5 mL) at 0° C. dropwise. The reaction mixture was stirred for 12 h at 25° C. After completed, the reaction was quenched with water and extracted with DCM (3×30 mL). The organic phase was washed with citric acid (sat, 10 mL), brine (2×30 mL), dried over Na₂SO₄ and concentrated under vacuum. The residue was purified by column chromatography on silica gel eluting with methanol in dichloromethane (6%) to afford the title compound (1.3 g, impure). ¹H NMR (400 MHz, DMSO-d₆) δ 9.07 (s, 1H), 8.60 (s, 1H), 8.21 (s, 1H), 4.54 (s, 2H), 3.93 (s, 3H), 1.44 (s, 9H), 1.12 (s, 3H), 0.85-0.76 (m, 2H), 0.64-0.56 (m, 2H) ppm. MS: M/e 361. (M+H)⁺.

### Step F: 6-(((tert-butoxycarbonyl)(1-methylcyclopropyl)amino)methyl)-[1,2,4]triazolo[1,5-a]pyridine-8-carboxylic acid

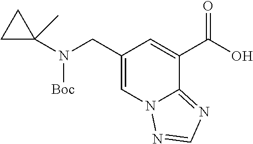 [SMILES: CC(C)(C)OC(=O)N(Cc1cc(C(=O)O)c2ncnn2c1)C1(C)CC1]

To a solution of methyl 6-(((tert-butoxycarbonyl)(1-methylcyclopropyl)amino)methyl)-[1,2,4]triazolo[1,5-a]pyridine-8-carboxylate (150 mg, impure) in MeOH (5 mL) was added LiOH H₂O (35 mg, 0.833 mmol) in H₂O (0.5 mL). The reaction mixture was stirred for 2 h at 25° C. After completed, the solvent was concentrated under vacuum. The residue was diluted with water (10 mL) and neutralized with 6N HCl at 0° C. to adjust PH=7 and the mixture was freeze dried. The crude product (150 mg, lithium salt) was obtained. MS: M/e 347 (M+H)⁺.

### Step G: tert-butyl ((8-((3-((1s, 3s)-3-methyl-1-(4-methyl-4H-1,2,4-triazol-3-yl)cyclobutyl) phenyl)carbamoyl)-[1,2,4]triazolo[1,5-a]pyridin-6-yl)methyl)(1-methylcyclopropyl)carbamate

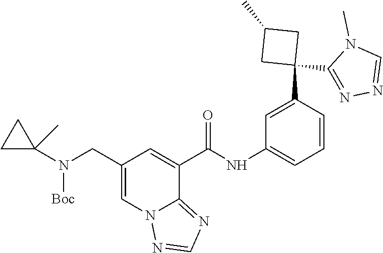 [SMILES: Cn1cnnc1[C@]1(c2cccc(NC(=O)c3cc(CN(C(=O)OC(C)(C)C)C4(C)CC4)cn4ncnc34)c2)C[C@H](C)C1]

To a solution of 6-(((tert-butoxycarbonyl)(1-methylcyclopropyl)amino)methyl)-[1,2,4]triazolo[1,5-a]pyridine-8-carboxylic acid (150 mg, crude) and 3-((1s, 3s)-3-methyl-1-(4-methyl-4H-1,2,4-triazol-3-yl)cyclobutyl)aniline (100 mg, 0.413 mmol) in DMF (2 mL) was added DIEA (110 mg, 0.853 mmol) and HATU (235 mg, 0.618 mmol). The reaction mixture was stirred for 1 h at 25° C. After completed, the reaction was quenched with water (10 mL) and extracted with DCM (2×20 mL). The organic phase was washed with aq NaHCO₃ (20 mL), brine (2×20 mL), dried over Na₂SO₄ and concentrated under vacuum. The residue was purified by column chromatography on silica gel eluting with methanol in dichloromethane (6%) to afford the title compound (210 mg, impure). MS: M/e 571. (M+H)⁺.

### Step H: N-(3-((1s, 3s)-3-methyl-1-(4-methyl-4H-1,2,4-triazol-3-yl)cyclobutyl)phenyl)-6-(((1-methylcyclopropyl)amino)methyl)-[1,2,4]triazolo[1,5-a]pyridine-8-carboxamide

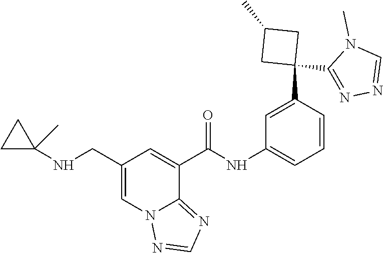 [SMILES: Cn1cnnc1[C@]1(c2cccc(NC(=O)c3cc(CNC4(C)CC4)cn4ncnc34)c2)C[C@H](C)C1]

To a solution of tert-butyl ((8-((3-((1s, 3s)-3-methyl-1-(4-methyl-4H-1,2,4-triazol-3-yl)cyclobutyl)phenyl)carbamoyl)-[1,2,4]triazolo[1,5-a]pyridin-6-yl)methyl)(1-methylcyclopropyl)carbamate (210 mg) in DCM (0.5 mL) was added HCl (5 mL, 4 M in dioxane). The reaction mixture was stirred for 2 h at 25° C. The solid formed during the reaction. After completed, the reaction mixture was filtrated and the solid was washed with EtOAc (2×5 mL). The solid was diluted with DCM (20 mL) and quenched with aq NaHCO₃ to adjust PH=8-9 and extracted with DCM (2×20 mL), dried over Na₂SO₄ and concentrated under vacuum. The solid was diluted with MeOH/Water to give the title compound (110 mg, 57% for two steps). ¹H NMR (400 MHz, DMSO-d₆) δ 11.41 (s, 1H), 9.08 (s, 1H), 8.74 (s, 1H), 8.38 (s, 1H), 8.27 (s, 1H), 7.71-7.64 (m, 2H), 7.40 (t, J=8.2 Hz, 1H), 7.15 (d, J=8.0 Hz, 1H), 3.89 (s, 2H), 3.17 (s, 3H), 2.87-2.75 (m, 2H), 2.58-2.48 (m, 4H), 1.25 (s, 3H), 1.06 (d, J=5.2 Hz, 3H), 0.54-0.46 (m, 2H), 0.35-0.27 (m, 2H) ppm. MS: M/e 471 (M+H)⁺.

### Compound A88: N-(5-((1s, 3s)-3-methyl-1-(4-methyl-4H-1,2,4-triazol-3-yl)cyclobutyl)pyridin-3-yl)-6-(((1-methylcyclopropyl)amino)methyl)-[1,2,4]triazolo[1,5-a]pyridine-8-carboxamide

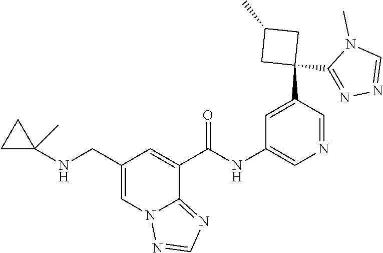 [SMILES: Cn1cnnc1[C@]1(c2cncc(NC(=O)c3cc(CNC4(C)CC4)cn4ncnc34)c2)C[C@H](C)C1]

### Step A: tert-butyl ((8-((5-((1s, 3s)-3-methyl-1-(4-methyl-4H-1,2,4-triazol-3-yl)cyclobutyl)pyridin-3-yl)carbamoyl)-[1,2,4]triazolo[1,5-a]pyridin-6-yl)methyl)(1-methylcyclopropyl)carbamate

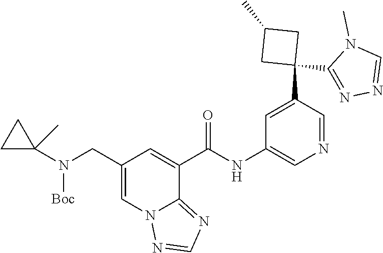 [SMILES: Cn1cnnc1[C@]1(c2cncc(NC(=O)c3cc(CN(C(=O)OC(C)(C)C)C4(C)CC4)cn4ncnc34)c2)C[C@H](C)C1]

To a solution of 6-(((tert-butoxycarbonyl)(1-methylcyclopropyl)amino)methyl)-[1,2,4]triazolo[1,5-a]pyridine-8-carboxylic acid (150 mg, crude) and 5-((1s, 3s)-3-methyl-1-(4-methyl-4H-1,2,4-triazol-3-yl)cyclobutyl)pyridin-3-amine (100 mg, 0.411 mmol) in DMF (2 mL) was added DIEA (110 mg, 0.853 mmol) and HATU (235 mg, 0.618 mmol). The reaction mixture was stirred for 1 h at 25° C. After completed, the reaction was quenched with water (10 mL) and extracted with DCM (2×20 mL). The organic phase was washed with aq NaHCO₃ (20 mL), brine (2×20 mL), dried over Na₂SO₄ and concentrated under vacuum. The residue was purified by column chromatography on silica gel eluting with methanol in dichloromethane (6%) to afford the title compound (196 mg, impure). MS: M/e 572 (M+H)⁺.

### Step B: N-(5-((1s, 3s)-3-methyl-1-(4-methyl-4H-1,2,4-triazol-3-yl)cyclobutyl)pyridin-3-yl)-6-(((1-methylcyclopropyl)amino)methyl)-[1,2,4]triazolo[1,5-a]pyridine-8-carboxamide

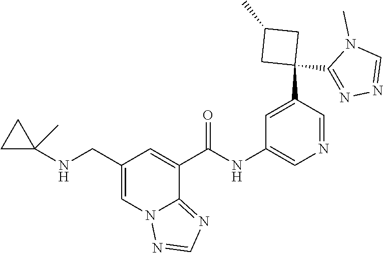 [SMILES: Cn1cnnc1[C@]1(c2cncc(NC(=O)c3cc(CNC4(C)CC4)cn4ncnc34)c2)C[C@H](C)C1]

To a solution of tert-butyl ((8-((5-((1s, 3s)-3-methyl-1-(4-methyl-4H-1,2,4-triazol-3-yl)cyclobutyl)pyridin-3-yl)carbamoyl)-[1,2,4]triazolo[1,5-a]pyridin-6-yl)methyl)(1-methylcyclopropyl)carbamate (196 mg) in DCM (0.5 mL) was added HCl (5 mL, 4 M in dioxane). The reaction mixture was stirred for 2 h at 25° C. After completed, the reaction was quenched with aq NaHCO₃ (20 mL) and extracted with DCM (2×20 mL), dried over Na₂SO₄ and concentrated under vacuum. The residue was purified by Prep-HPLC to afford the title compound (60 mg, 310% for two steps). ¹H NMR (400 MHz, DMSO-d₆) δ 11.46 (s, 1H), 9.11 (s, 1H), 8.87 (d, J=2.0 Hz, 1H), 8.76 (s, 1H), 8.43 (d, J=1.6 Hz, 1H), 8.39 (s, 1H), 8.34 (s, 1H), 8.09 (s, 1H), 3.90 (s, 2H), 3.23 (s, 3H), 2.96-2.84 (m, 2H), 2.71 (s, 1H), 2.65-2.53 (m, 3H), 1.27 (s, 3H), 1.09 (d, J=4.8 Hz, 3H), 0.51 (t, J=4.8 Hz, 2H), 0.32 (q, J=4.0 Hz, 2H) ppm. MS: M/e 472 (M+H)⁺.

### Compound A89: N-(3-((1s, 3s)-3-(cyanomethyl)-1-(4-methyl-4H-1,2,4-triazol-3-yl)cyclobutyl)phenyl)-6-(((1-methylcyclopropyl)amino)methyl)-[1,2,4]triazolo[1,5-a]pyridine-8-carboxamide

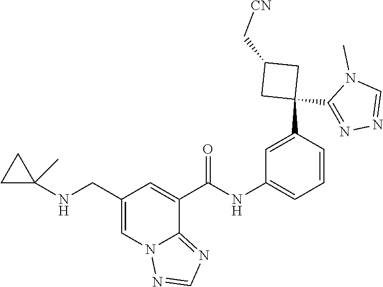 [SMILES: Cn1cnnc1[C@]1(c2cccc(NC(=O)c3cc(CNC4(C)CC4)cn4ncnc34)c2)C[C@H](CC#N)C1]

### Step A: tert-butyl ((8-((3-((1s, 3s)-3-(cyanomethyl)-1-(4-methyl-4H-1,2,4-triazol-3-yl)cyclobutyl) phenyl)carbamoyl)-[1,2,4]triazolo[1,5-a]pyridin-6-yl)methyl)(1-methylcyclopropyl)carbamate

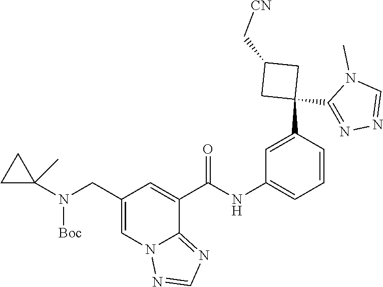 [SMILES: Cn1cnnc1[C@]1(c2cccc(NC(=O)c3cc(CN(C(=O)OC(C)(C)C)C4(C)CC4)cn4ncnc34)c2)C[C@H](CC#N)C1]

To a solution of 6-(((tert-butoxycarbonyl)(1-methylcyclopropyl)amino)methyl)-[1,2,4]triazolo[1,5-a]pyridine-8-carboxylic acid (150 mg, crude) and 2-((1s, 3s)-3-(3-aminophenyl)-3-(4-methyl-4H-1,2,4-triazol-3-yl)cyclobutyl)acetonitrile (111 mg, 0.415 mmol) in DMF (2 mL) was added DIEA (110 mg, 0.853 mmol) and HATU (235 mg, 0.618 mmol). The reaction mixture was stirred for 1 h at 25° C. After completed, the reaction was quenched with water (10 mL) and extracted with DCM (2×20 mL). The organic phase was washed with aq NaHCO₃ (20 mL), brine (2×20 mL), dried over Na₂SO₄ and concentrated under vacuum. The residue was purified by column chromatography on silica gel eluting with methanol in dichloromethane (6%) to afford the title compound (230 mg, impure). MS: M/e 596 (M+H)⁺.

### Step B: N-(3-((1s, 3s)-3-(cyanomethyl)-1-(4-methyl-4H-1,2,4-triazol-3-yl)cyclobutyl)phenyl)-6-(((1-methylcyclopropyl)amino)methyl)-[1,2,4]triazolo[1,5-a]pyridine-8-carboxamide

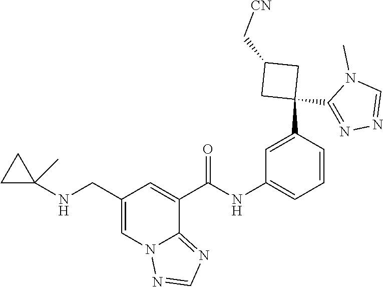 [SMILES: Cn1cnnc1[C@]1(c2cccc(NC(=O)c3cc(CNC4(C)CC4)cn4ncnc34)c2)C[C@H](CC#N)C1]

To a solution of tert-butyl ((8-((3-((1s, 3s)-3-(cyanomethyl)-1-(4-methyl-4H-1,2,4-triazol-3-yl)cyclobutyl)phenyl)carbamoyl)-[1,2,4]triazolo[1,5-a]pyridin-6-yl)methyl)(1-methylcyclopropyl)carbamate (230 mg) in DCM (0.5 mL) was added HCl (5 mL, 4 M in dioxane). The reaction mixture was stirred for 2 h at 25° C. After completed, the reaction was quenched with aq NaHCO₃ (20 mL) and extracted with DCM (2×20 mL), dried over Na₂SO₄ and concentrated under vacuum. The residue was purified by Prep-HPLC to afford the title compound (60 mg, 29% for two steps). ¹H NMR (400 MHz, DMSO-d₆) δ 11.44 (s, 1H), 9.10 (s, 1H), 8.76 (s, 1H), 8.39 (d, J=1.2 Hz, 1H), 8.33 (s, 1H), 7.72 (d, J=8.2 Hz, 2H), 7.45 (t, J=7.8 Hz, 1H), 7.22 (d, J=8.0 Hz, 1H), 3.90 (s, 2H), 3.22 (s, 3H), 2.94-2.83 (m, 2H), 2.82-2.62 (m, 6H), 1.27 (s, 3H), 0.51 (t, J=4.8 Hz, 2H), 0.32 (q, J=4.0 Hz, 2H). MS: M/e 496. (M+H)⁺.

### Compound A90: N-(3-((1r, 3r)-3-methyl-1-(4-methyl-4H-1,2,4-triazol-3-yl)cyclobutyl)phenyl)-6-(((1-methylcyclopropyl)amino)methyl)imidazo[1,2-a]pyridine-8-carboxamide

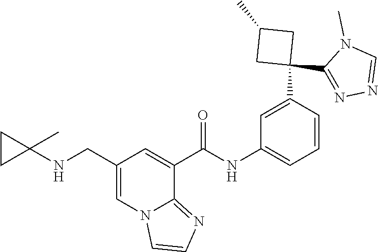 [SMILES: Cn1cnnc1[C@]1(c2cccc(NC(=O)c3cc(CNC4(C)CC4)cn4ccnc34)c2)C[C@@H](C)C1]

### Step 1: tert-butyl ((8-((3-((1r, 3r)-3-methyl-1-(4-methyl-4H-1,2,4-triazol-3-yl)cyclobutyl)phenyl)carbamoyl)imidazo[1,2-a]pyridin-6-yl)methyl)(1-methylcyclopropyl)carbamate

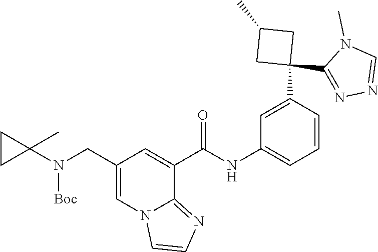 [SMILES: Cn1cnnc1[C@]1(c2cccc(NC(=O)c3cc(CN(C(=O)OC(C)(C)C)C4(C)CC4)cn4ccnc34)c2)C[C@@H](C)C1]

To a solution of 6-(((tert-butoxycarbonyl)(1-methylcyclopropyl)amino)methyl)imidazo[1,2-a]pyridine-8-carboxylic acid (35.63 mg, 103.17 mol) in DMF (1 mL) was added DIEA (26.67 mg, 206.34 mol) and HATU (47.07 mg, 123.80 mol). The mixture was stirred for 5 mins at 20° C., then 3-((1r, 3r)-3-methyl-1-(4-methyl-4H-1,2,4-triazol-3-yl)cyclobutyl)aniline (25 mg, 103.17 μmol) was added. The mixture was stirred for 2 hr at 20° C. The reaction mixture was added H2O 1 mL at 20° C., and then extracted with DCM (1 mL*3) mL. The combined organic layers were dried over Na2SO4, filtered and concentrated under reduced pressure to give a residue, which was purified by prep-HPLC (column: Waters Xbridge C18 150*50 mm*10 um; mobile phase: [H2O (10 mM NH₄HCO3)-ACN]; gradient: 40%-70% B over 8.0 min) to give the product (25 mg, 42.53% yield). MS: M/e 570 (M+1).

### Step 2: N-(3-((1r, 3r)-3-methyl-1-(4-methyl-4H-1,2,4-triazol-3-yl)cyclobutyl)phenyl)-6-(((1-methylcyclopropyl)amino)methyl)imidazo[1,2-a]pyridine-8-carboxamide

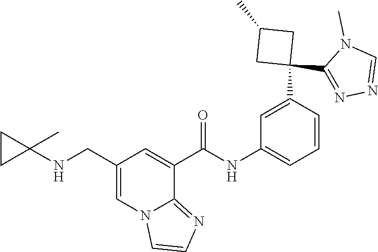 [SMILES: Cn1cnnc1[C@]1(c2cccc(NC(=O)c3cc(CNC4(C)CC4)cn4ccnc34)c2)C[C@@H](C)C1]

A mixture of tert-butyl ((8-((3-((1r, 3r)-3-methyl-1-(4-methyl-4H-1,2,4-triazol-3-yl)cyclobutyl)phenyl)carbamoyl)imidazo[1,2-a]pyridin-6-yl)methyl)(1-methylcyclopropyl)carbamate (20 mg, 35.11 μmol) in DCM (1 mL) and TFA (0.5 mL) was degassed and purged with N2 for 3 times, and then the mixture was stirred at 20° C. for 3 hr under N2 atmosphere. The reaction mixture was concentrated. Then the aqueous phase was adjusted to pH 8 with saturated aqueous Na2CO3, and then extracted with DCM 3 mL (1 mL*3). The combined organic layers were washed with brine 2 mL (1 mL*2), dried over Na2SO4, filtered and concentrated under reduced pressure to give a residue, which was purified by prep-HPLC (column: Waters Xbridge BEH C18 100*30 mm*10 um; mobile phase: [H2O (10 mM NH₄HCO3)-ACN]; gradient: 25%-65% B over 8.0 min) to give the product (10.19 mg, 61.81% yield). MS: M/e 470 (M+1); ¹H NMR (400 MHz, DMSO-d₆) δ=12.40 (s, 1H), 8.71 (s, 1H), 8.37 (s, 1H), 8.12 (d, J=1.2 Hz, 1H), 8.10 (d, J=1.6 Hz, 1H), 7.77 (d, J=1.2 Hz, 1H), 7.65-7.57 (m, 2H), 7.39 (t, J=8.0 Hz, 1H), 6.99 (d, J=8.0 Hz, 1H), 3.81 (s, 2H), 3.23 (s, 3H), 3.16-3.06 (m, 2H), 2.41-2.30 (m, 1H), 2.29-2.19 (m, 2H), 1.26 (s, 3H), 1.10 (d, J=6.4 Hz, 3H), 0.55-0.46 (m, 2H), 0.38-0.28 (m, 2H).

### Compound A91: N-(3-((1s, 3s)-3-methyl-1-(4-methyl-4H-1,2,4-triazol-3-yl)cyclobutyl)phenyl)-5-(((1-methylcyclopropyl)amino)methyl)pyrazolo[1,5-a]pyridine-7-carboxamide

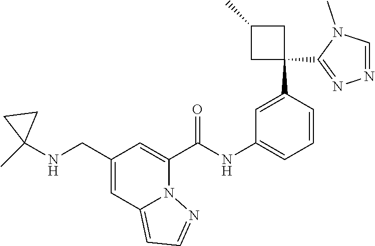 [SMILES: Cn1cnnc1[C@]1(c2cccc(NC(=O)c3cc(CNC4(C)CC4)cc4ccnn34)c2)C[C@H](C)C1]

### Step 1: 5-(((1-methylcyclopropyl)amino)methyl)pyrazolo[1,5-a]pyridine-7-carbonitrile

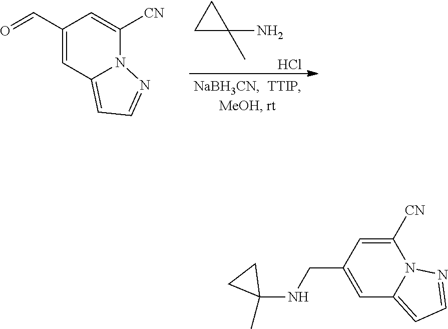

A mixture of 5-formylpyrazolo[1,5-a]pyridine-7-carbonitrile (430 mg, 2.236 mmol, 1.0 equiv, 89%) and 1-methylcyclopropan-1-amine hydrochloride (506 mg, 4.468 mmol, 2.0 equiv, 95%) in MeOH (10 mL) was treated with tetrakis(propan-2-yloxy)titanium (2.68 g, 8.944 mmol, 4.0 equiv, 95%) for 3 h at room temperature under nitrogen atmosphere followed by the addition of NaBH₃CN (192 mg, 2.903 mmol, 1.3 equiv, 95%) dropwise at room temperature. The resulting mixture was stirred for 1 h at room temperature under nitrogen atmosphere. The resulting mixture was concentrated under vacuum. The residue was purified by silica gel column chromatography, eluted with CH₂Cl₂/MeOH (10:1) to afford 5-(((1-methylcyclopropyl)amino)methyl)pyrazolo[1,5-a]pyridine-7-carbonitrile (230 mg, 40.91%). MS: M/e 227 (M+1).

### Step 2: 5-(((1-methylcyclopropyl)amino)methyl)pyrazolo[1,5-a]pyridine-7-carboxylic acid

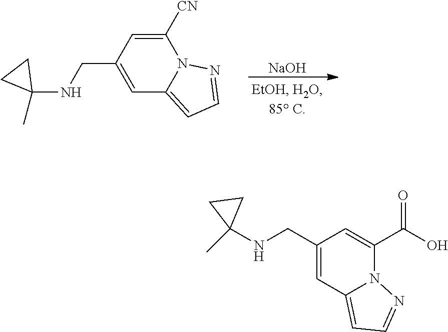

To a stirred solution of 5-([(1-methylcyclopropyl)amino]methylpyrazolo[1,5-a]pyridine-7-carbonitrile (200 mg, 0.795 mmol, 1.0 equiv, 90%) in H₂O (2 mL)/EtOH (2 mL) was added NaOH (134 mg, 3.183 mmol, 4.0 equiv, 95%) in portions at room temperature. The resulting mixture was stirred for 16 h at 85° C. under nitrogen atmosphere. The resulting mixture was concentrated under vacuum. The residue was purified by silica gel column chromatography, eluted with CH₂Cl₂/MeOH (10:1) to afford 5-(((1-methylcyclopropyl)amino)methyl)pyrazolo[1,5-a]pyridine-7-carboxylic acid (120 mg, 60.89%). MS: M/e 246 (M+1).

### Step 3: N-(3-((1s, 3s)-3-methyl-1-(4-methyl-4H-1,2,4-triazol-3-yl)cyclobutyl)phenyl)-5-(((1-methylcyclopropyl)amino)methyl)pyrazolo[1,5-a]pyridine-7-carboxamide

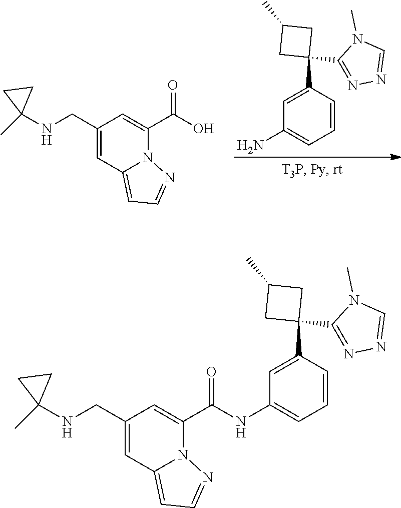 [SMILES: CC1(NCc2cc(C(=O)O)n3nccc3c2)CC1 ; Cn1cnnc1[C@]1(c2cccc(N)c2)C[C@H](C)C1 ; Cn1cnnc1[C@]1(c2cccc(NC(=O)c3cc(CNC4(C)CC4)cc4ccnn34)c2)C[C@H](C)C1]

To a stirred mixture of 5-(((1-methylcyclopropyl)amino)methylpyrazolo[1,5-a]pyridine-7-carboxylic acid (100 mg, 0.404 mmol, 1.0 equiv, 99%) and 3-[(1r, 3s)-3-methyl-1-(4-methyl-1,2,4-triazol-3-yl)cyclobutyl]aniline (103 mg, 0.404 mmol, 1.0 equiv, 95%) in Pyridine (3 mL) was added T₃P (1.35 g, 4.031 mmol, 10.0 equiv, 95%) dropwise at room temperature. The resulting mixture was stirred for 1 h at room temperature under nitrogen atmosphere. The resulting mixture was concentrated under vacuum. The crude product was purified by Prep-HPLC with the following conditions (Column: XBridge Prep OBD C18 Column, 30*150 mm, 5 μm; Mobile Phase A: Water (10 mmol/L NH₄HCO₃+0.05% NH₃·H₂O), Mobile Phase B: ACN; Flow rate: 60 mL/min mL/min; Gradient: 35% B to 65% B in 8 min; Wave Length: 254 nm; RT1 (min): 7) to afford 5-([(1-methylcyclopropyl)amino]methyl-N-(3-[(1r, 3s)-3-methyl-1-(4-methyl-1,2,4-triazol-3-yl)cyclobutyl]phenylpyrazolo[1,5-a]pyridine-7-carboxamide (11.6 mg, 5.99%). MS: M/e 470 (M+1); ¹H NMR (300 MHz, DMSO-d₆) δ 12.51 (s, 1H), 8.30 (s, 1H), 8.23 (d, J=2.4 Hz, 1H), 7.87 (d, J=1.8 Hz, 1H), 7.77 (m, 2H), 7.67 (m, J=7.9, 2.0 Hz, 1H), 7.43 (t, J=7.9 Hz, 1H), 7.16 (d, J=7.8 Hz, 1H), 6.82 (d, J=2.5 Hz, 1H), 3.85 (s, 2H), 3.20 (s, 3H), 2.88-2.78 (m, 2H), 2.55 (d, J=6.4 Hz, 3H), 1.25 (d, J=9.3 Hz, 3H), 1.09 (d, J=5.0 Hz, 3H), 0.53 (q, J=3.9 Hz, 2H), 0.32 (q, J=3.9 Hz, 2H).

### Compound A92: 3-fluoro-N-(3-((1s, 3s)-3-methyl-1-(4-methyl-4H-1,2,4-triazol-3-yl)cyclobutyl)phenyl)-5-(((1-methylcyclopropyl)amino)methyl)pyrazolo[1,5-a]pyridine-7-carboxamide

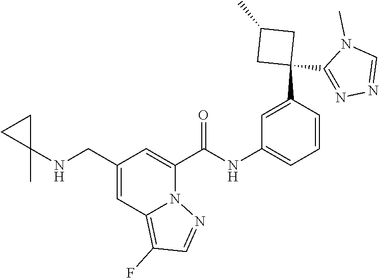 [SMILES: Cn1cnnc1[C@]1(c2cccc(NC(=O)c3cc(CNC4(C)CC4)cc4c(F)cnn34)c2)C[C@H](C)C1]

### Step 1: 3-fluoro-5-(((1-methylcyclopropyl)amino)methyl)pyrazolo[1,5-a]pyridine-7-carbonitrile

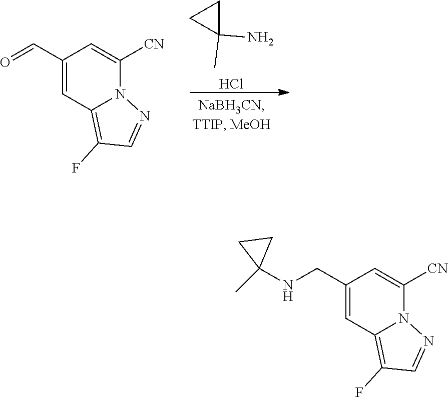

To a stirred mixture of 3-fluoro-5-formylpyrazolo[1,5-a]pyridine-7-carbonitrile (600 mg, 3.004 mmol, 1 equiv, 94.7%) and 1-methylcyclopropan-1-amine hydrochloride (681 mg, 6.014 mmol, 2.00 equiv, 95%) in MeOH (15 mg, 99%) was added tetrakis(propan-2-yloxy)titanium (3.6 g, 12.033 mmol, 4.01 equiv, 95%) dropwise. The resulting mixture was stirred for 3 h at room temperature. To the above mixture was added NaBH₃CN (398 mg, 6.017 mmol, 2.00 equiv, 95%) in portions at room temperature. The resulting mixture was stirred for additional 1 h at room temperature. The resulting mixture was concentrated under vacuum. The residue was purified by silica gel column chromatography, eluted with CH₂Cl₂/MeOH (12:1) to afford 3-fluoro-5-(((1-methylcyclopropyl)amino)methyl)pyrazolo[1,5-a]pyridine-7-carbonitrile (600 mg, 69.75%). MS: M/e 245 (M+1).

### Step 2: 3-fluoro-5-(((1-methylcyclopropyl)amino)methyl)pyrazolo[1,5-a]pyridine-7-carboxylic acid

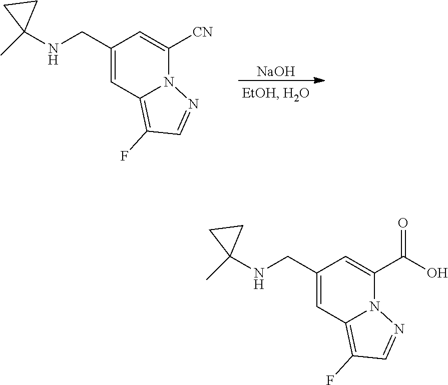 [SMILES: CC1(NCc2cc(C#N)n3ncc(F)c3c2)CC1 ; "NaOH" ; CC1(NCc2cc(C(=O)O)n3ncc(F)c3c2)CC1]

To a stirred solution of 3-fluoro-5-([(1-methylcyclopropyl)amino]methylpyrazolo[1,5-a]pyridine-7-carbonitrile (500 mg, 1.746 mmol, 1 equiv, 85.3%) in EtOH (10 mL, 99%) and H₂O (8 mL, 99%) was added NaOH (221 mg, 5.249 mmol, 3.01 equiv, 95%) in portions. The resulting mixture was stirred for 3 h at 80° C. The mixture was allowed to cool down to room temperature. The resulting mixture was concentrated under vacuum. The residue was acidified to pH 4 with HCl (1 M). The precipitated solids were collected by filtration and washed with water (2×2 mL). This resulted in 3-fluoro-5-(((1-methylcyclopropyl)amino)methyl)pyrazolo[1,5-a]pyridine-7-carboxylic acid (350 mg, 73.63%). MS: M/e 264 (M+1).

### Step 3: 3-fluoro-N-(3-((1s, 3s)-3-methyl-1-(4-methyl-4H-1,2,4-triazol-3-yl)cyclobutyl)phenyl)-5-(((1-methylcyclopropyl)amino)methyl)pyrazolo[1,5-a]pyridine-7-carboxamide

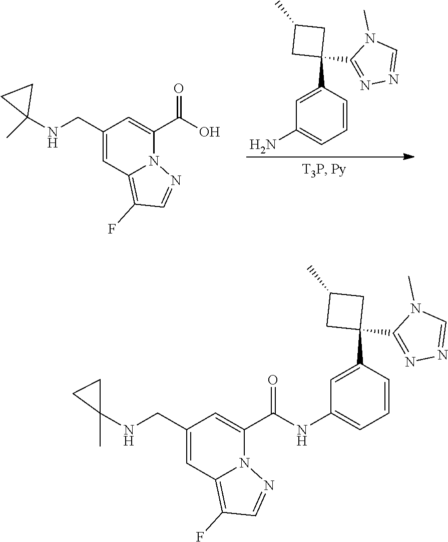 [SMILES: CC1(NCc2cc(C(=O)O)n3ncc(F)c3c2)CC1 ; Cn1cnnc1[C@]1(c2cccc(N)c2)C[C@H](C)C1 ; Cn1cnnc1[C@]1(c2cccc(NC(=O)c3cc(CNC4(C)CC4)cc4c(F)cnn34)c2)C[C@H](C)C1]

To a stirred mixture of 3-fluoro-5-([(1-methylcyclopropyl)amino]methylpyrazolo[1,5-a]pyridine-7-carboxylic acid (100 mg, 0.367 mmol, 1 equiv, 96.7%) and 3-[(1r, 3s)-3-methyl-1-(4-methyl-1,2,4-triazol-3-yl)cyclobutyl]aniline (109 mg, 0.368 mmol, 1.00 equiv, 81.8%) in Pyridine (3 mL, 95%) was added T₃P (1.9 g, 2.986 mmol, 8.13 equiv, 50%) dropwise at room temperature. The resulting mixture was stirred for 1 h at room temperature. The resulting mixture was concentrated under vacuum. The crude product (200 mg) was purified by Prep-HPLC with the following conditions (2 #SHIMADZU (HPLC-01)): Column, YMC-Actus Triart C18 ExRS, 30*150 mm, 5 μm; mobile phase, 10 mmol NH₄HCO₃+0.1% NH₃H2O and ACN (45% ACN up to 75% in 9 min); This resulted in 3-fluoro-5-([(1-methylcyclopropyl)amino]methyl-N-(3-[(1r, 3s)-3-methyl-1-(4-methyl-1,2,4-triazol-3-yl)cyclobutyl]phenylpyrazolo[1,5-a]pyridine-7-carboxamide (26.6 mg, 14.64%). MS: M/e 488 (M+1). ¹H NMR (300 MHz, DMSO-d₆) δ 11.93 (s, 1H), 8.36-8.27 (m, 2H), 7.84-7.77 (m, 1H), 7.75-7.63 (m, 3H), 7.47-7.36 (m, 1H), 7.17 (d, J=7.7 Hz, 1H), 3.85 (s, 2H), 3.19 (s, 3H), 2.85-2.79 (m, 3H), 2.59-2.51 (m, 2H), 1.26 (s, 3H), 1.09 (d, J=5.1 Hz, 3H), 0.58-0.48 (m, 2H), 0.37-0.28 (m, 2H).

### Compound A93: 3-fluoro-N-(5-((1s, 3s)-3-methyl-1-(4-methyl-4H-1,2,4-triazol-3-yl)cyclobutyl)pyridin-3-yl)-5-(((1-methylcyclopropyl)amino)methyl)pyrazolo[1,5-a]pyridine-7-carboxamide

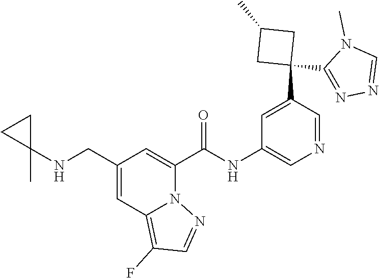 [SMILES: Cn1cnnc1[C@]1(c2cncc(NC(=O)c3cc(CNC4(C)CC4)cc4c(F)cnn34)c2)C[C@H](C)C1]

### Step 1: 3-fluoro-N-(5-((1s, 3s)-3-methyl-1-(4-methyl-4H-1,2,4-triazol-3-yl)cyclobutyl)pyridin-3-yl)-5-(((1-methylcyclopropyl)amino)methyl)pyrazolo[1,5-a]pyridine-7-carboxamide

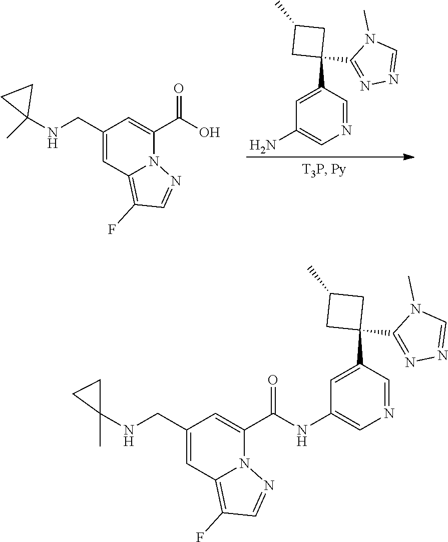 [SMILES: CC1(NCc2cc(C(=O)O)n3ncc(F)c3c2)CC1 ; Cn1cnnc1[C@]1(c2cncc(N)c2)C[C@H](C)C1 ; Cn1cnnc1[C@]1(c2cncc(NC(=O)c3cc(CNC4(C)CC4)cc4c(F)cnn34)c2)C[C@H](C)C1]

To a stirred mixture of 3-fluoro-5-([(1-methylcyclopropyl)amino]methylpyrazolo[1,5-a]pyridine-7-carboxylic acid (80 mg, 0.294 mmol, 1 equiv, 96.7%) and 5-[(1r, 3s)-3-methyl-1-(4-methyl-1,2,4-triazol-3-yl)cyclobutyl]pyridin-3-amine (80 mg, 0.296 mmol, 1.01 equiv, 90%) in Pyridine (3 mL, 0.036 mmol, 95%) were added T₃P (1.50 g, 2.352 mmol, 8 equiv, 50%) dropwise at room temperature. The resulting mixture was stirred for 1 h at room temperature. The resulting mixture was concentrated under vacuum. The crude product (200 mg) was purified by Prep-HPLC with the following conditions (2 #SHIMADZU (HPLC-01)): Column, YMC-Actus Triart C18 ExRS, 30*150 mm, 5 μm; mobile phase, 10 mmol NH₄HCO₃+0.05% NH₃H2O and ACN (40% ACN up to 70% in 9 min); This resulted in 3-fluoro-N-(5-((1s, 3s)-3-methyl-1-(4-methyl-4H-1,2,4-triazol-3-yl)cyclobutyl)pyridin-3-yl)-5-(((1-methylcyclopropyl)amino)methyl)pyrazolo[1,5-a]pyridine-7-carboxamide (50.8 mg, 35.21%). MS: M/e 489 (M+1). ¹H NMR (300 MHz, DMSO-d₆) δ 12.04 (s, 1H), 8.87 (d, J=2.2 Hz, 1H), 8.43 (d, J=2.1 Hz, 1H), 8.38-8.29 (m, 2H), 8.14-8.07 (m, 1H), 7.87-7.80 (m, 1H), 7.68 (d, J=1.8 Hz, 1H), 3.87 (s, 2H), 3.24 (s, 3H), 2.94-2.86 (m, 2H), 2.63-2.55 (m, 3H), 1.27 (s, 3H), 1.10 (d, J=5.1 Hz, 3H), 0.59-0.50 (m, 2H), 0.39-0.29 (m, 2H).

### Compound A94: N-(3-((1s, 3s)-3-(cyanomethyl)-1-(4-methyl-4H-1,2,4-triazol-3-yl)cyclobutyl)phenyl)-3-fluoro-5-(((1-methylcyclopropyl)amino)methyl)pyrazolo[1,5-a]pyridine-7-carboxamide

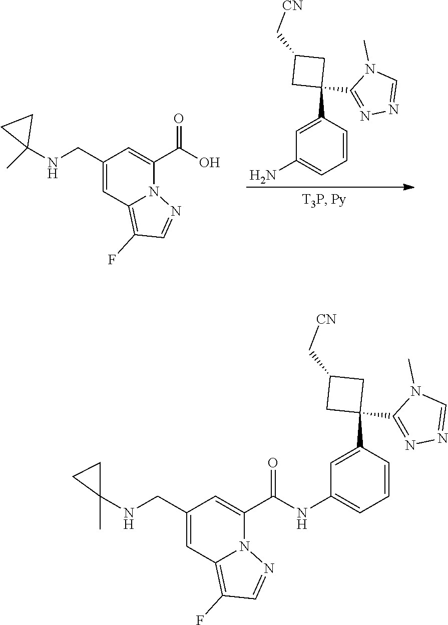 [SMILES: CC1(NCc2cc(C(=O)O)n3ncc(F)c3c2)CC1 ; Cn1cnnc1[C@]1(c2cccc(N)c2)C[C@H](CC#N)C1 ; Cn1cnnc1[C@]1(c2cccc(NC(=O)c3cc(CNC4(C)CC4)cc4c(F)cnn34)c2)C[C@H](CC#N)C1]

To a stirred mixture of 3-fluoro-5-([(1-methylcyclopropyl)amino]methylpyrazolo[1,5-a]pyridine-7-carboxylic acid (80 mg, 0.294 mmol, 1 equiv, 96.7%) and 2-[(1s, 3s)-3-(3-aminophenyl)-3-(4-methyl-1,2,4-triazol-3-yl)cyclobutyl]acetonitrile (80 mg, 0.294 mmol, 1.00 equiv, 98.3%) in Pyridine (3 mL, 95%) were added T₃P (1.50 g, 2.352 mmol, 8 equiv, 50%) dropwise at room temperature. The resulting mixture was stirred for 1 h at room temperature. The resulting mixture was concentrated under vacuum. The crude product (200 mg) was purified by Prep-HPLC with the following conditions (2 #SHIMADZU (HPLC-01)): Column, YMC-Actus Triart C18 ExRS, 30*150 mm, 5 μm; mobile phase, 10 mmol NH₄HCO₃+0.1% NH₃H2O and ACN (35% ACN up to 65% in 9 min); This resulted in N-(3-((1s, 3s)-3-(cyanomethyl)-1-(4-methyl-4H-1,2,4-triazol-3-yl)cyclobutyl)phenyl)-3-fluoro-5-(((1-methylcyclopropyl)amino)methyl)pyrazolo[1,5-a]pyridine-7-carboxamide (16.9 mg, 11.05%). MS: M/e 513 (M+1). ¹H NMR (300 MHz, DMSO-d₆) δ 11.93 (s, 1H), 8.36-8.29 (m, 2H), 7.84-7.77 (m, 1H), 7.75-7.63 (m, 3H), 7.50-7.38 (m, 1H), 7.21 (d, J=7.8 Hz, 1H), 3.85 (s, 2H), 3.21 (s, 3H), 2.91-2.67 (m, 7H), 1.26 (s, 3H), 0.57-0.48 (m, 2H), 0.37-0.28 (m, 2H).

### Compound A95: N-(3-((1s, 3s)-3-methyl-1-(4-(methyl-d3)-4H-1,2,4-triazol-3-yl)cyclobutyl)phenyl)-6-(((1-methylcyclopropyl)amino)methyl)imidazo[1,2-a]pyridine-8-carboxamide

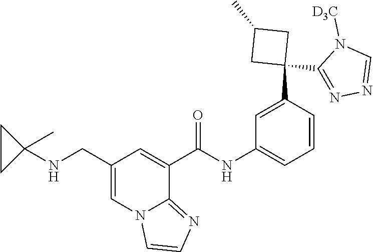 [SMILES: [2H]C([2H])([2H])n1cnnc1[C@]1(c2cccc(NC(=O)c3cc(CNC4(C)CC4)cn4ccnc34)c2)C[C@H](C)C1]

To a solution of 6-(((1-methylcyclopropyl)amino)methyl)imidazo[1,2-a]pyridine-8-carboxylic acid (93 mg, 0.380 mmol), 3-((1s, 3s)-3-methyl-1-(4-(methyl-d3)-4H-1,2,4-triazol-3-yl)cyclobutyl)aniline (93 mg, 0.380 mmol) in pyridine (3 mL) was added T₃P (725 mg, 1.140 mmol, 50% in EA). The mixture solution was stirred for 2 h at 25° C. After completed, the reaction was quenched with aq NH₄Cl (2 mL) and extracted with EA (2×10 mL), dried over Na₂SO₄ and concentrated under vacuum. The crude product was purified by Prep-HPLC to give the title compound (30 mg, 17%). MS: M/e 473 (M+1)⁺. H NMR (400 MHz, DMSO-d₆) δ 12.41 (s, 1H), 8.72 (s, 1H), 8.31 (s, 1H), 8.14 (s, 1H), 8.11 (s, 1H), 7.78 (s, 1H), 7.73 (s, 1H), 7.67 (d, J=8.0 Hz, 1H), 7.42 (t, J=7.9 Hz, 1H), 7.16 (d, J=7.7 Hz, 1H), 3.83 (s, 2H), 2.90-2.80 (m, 2H), 2.54 (d, J=5.8 Hz, 3H), 1.27 (s, 3H), 1.08 (d, J=4.4 Hz, 3H), 0.52 (s, 2H), 0.33 (s, 2H) ppm.

### Compound A96: 3-chloro-N-(3-((1s, 3s)-3-methyl-1-(4-methyl-4H-1,2,4-triazol-3-yl)cyclobutyl)phenyl)-5-(((1-methylcyclopropyl)amino)methyl)pyrazolo[1,5-a]pyridine-7-carboxamide

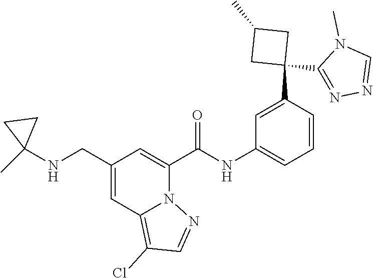 [SMILES: Cn1cnnc1[C@]1(c2cccc(NC(=O)c3cc(CNC4(C)CC4)cc4c(Cl)cnn34)c2)C[C@H](C)C1]

### Step 1: 7-(((1-methylcyclopropyl)amino)methyl)-1H-pyrrolo[3,2-b]pyridine-5-carbonitrile

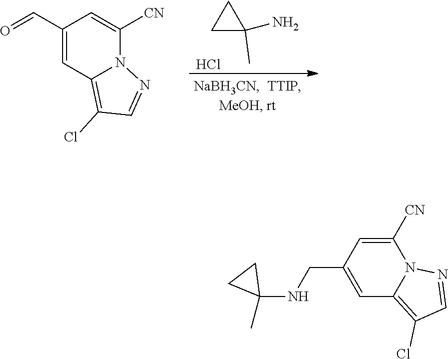

To a stirred solution of 3-chloro-5-formylpyrazolo[1,5-a]pyridine-7-carbonitrile (700 mg, 3.064 mmol, 1 equiv, 90%) and 1-methylcyclopropan-1-amine hydrochloride (693.99 mg, 6.128 mmol, 2 equiv, 95%) in MeOH was added tetrakis(propan-2-yloxy)titanium (3666.91 mg, 12.256 mmol, 4 equiv, 95%) at 0° C. under nitrogen atmosphere. The resulting mixture was stirred for additional 1 h at room temperature. To the above mixture was added NaBH₃CN (405.38 mg, 6.128 mmol, 2 equiv, 95%) at room temperature. The resulting mixture was stirred for additional 1 h at room temperature. The resulting mixture was concentrated under reduced pressure. The residue was purified by silica gel column chromatography, eluted with CH2Cl2/MeOH (10:1) to afford 3-chloro-5-([(1-methylcyclopropyl)amino]methylpyrazolo[1,5-a]pyridine-7-carbonitrile (510 mg, 52.35%) as a off-white solid. LC-MS (M+H)⁺:=261.

### Step 2: 3-chloro-5-(((1-methylcyclopropyl)amino)methyl)pyrazolo[1,5-a]pyridine-7-carboxylic acid

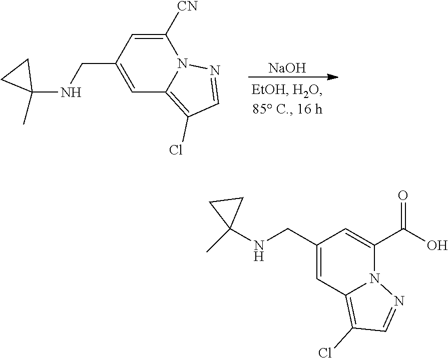

A solution of 3-chloro-5-([(1-methylcyclopropyl)amino]methylpyrazolo[1,5-a]pyridine-7-carbonitrile (460 mg, 1.447 mmol, 1 equiv, 82%) and NaOH (182.73 mg, 4.341 mmol, 3 equiv, 95%) in EtOH/H₂O was stirred for 3 h at 80° C. under nitrogen atmosphere. The mixture was acidified to pH 4 with HCl (aq.). The residue was purified by reversed-phase flash chromatography with the following conditions: column, C18 silica gel; mobile phase, MeCN in Water (0.1% TFA), 0% to 100% gradient in 30 min; detector, UV 254 nm. This resulted in 3-chloro-5-([(1-methylcyclopropyl)amino]methylpyrazolo[1,5-a]pyridine-7-carboxylic acid (210 mg, 46.70%) as a off-white solid. LC-MS (M+H)⁺:=280.

### Step 3: 3-chloro-N-(3-((1s, 3s)-3-methyl-1-(4-methyl-4H-1,2,4-triazol-3-yl)cyclobutyl)phenyl)-5-(((1-methylcyclopropyl)amino)methyl)pyrazolo[1,5-a]pyridine-7-carboxamide

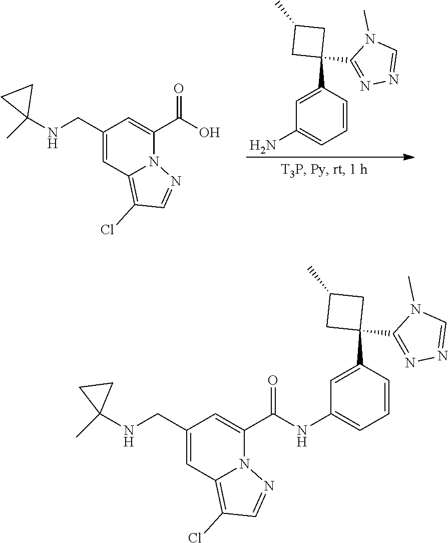 [SMILES: CC1(NCc2cc(C(=O)O)n3ncc(Cl)c3c2)CC1 ; Cn1cnnc1[C@]1(c2cccc(N)c2)C[C@H](C)C1 ; Cn1cnnc1[C@]1(c2cccc(NC(=O)c3cc(CNC4(C)CC4)cc4c(Cl)cnn34)c2)C[C@H](C)C1]

To a stirred solution of 3-chloro-5-([(1-methylcyclopropyl)amino]methylpyrazolo[1,5-a]pyridine-7-carboxylic acid (80 mg, 0.272 mmol, 1 equiv, 95%) and 3-[(1r, 3s)-3-methyl-1-(4-methyl-1,2,4-triazol-3-yl)cyclobutyl]aniline (83.17 mg, 0.326 mmol, 1.2 equiv, 95%) in pyridine was added T3P (1729.00 mg, 2.720 mmol, 10 equiv, 50%) dropwise at 0° C. under nitrogen atmosphere. The resulting mixture was stirred for additional 1 h at room temperature. The crude product was purified by Chiral-Prep-HPLC with the following conditions (2 #SHIMADZU (HPLC-01)): Column: XBridge Prep OBD C18 Column, 30*150 mm, 5 μm; mobile phase, 10 mmol NH4HCO3+0.05% NH3H2O and ACN (45% ACN up to 75% in 8 min); This resulted in 3-chloro-5-([(1-methylcyclopropyl)amino]methyl-N-(3-[(1r, 3s)-3-methyl-1-(4-methyl-1,2,4-triazol-3-yl)cyclobutyl]phenylpyrazolo[1,5-a]pyridine-7-carboxamide (45.3 mg, 32.09%). LC-MS (M+H)⁺:=504.20. ¹H NMR (300 MHz, DMSO-d6) δ 11.81 (s, 1H), 8.33 (m, 2H), 7.78-7.64 (m, 4H), 7.42 (m, 1H), 7.17 (m, 1H), 3.88 (s, 2H), 3.19 (s, 3H), 2.82 (s, 3H), 2.55 (m, 2H), 1.26 (s, 3H), 1.09 (m, 3H), 0.54 (m, 2H), 0.33 (m, 2H).

### Compound B1: 7-((isobutylamino)methyl)-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide

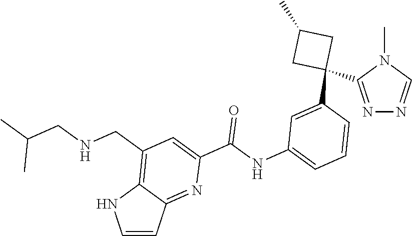 [SMILES: CC(C)CNCc1cc(C(=O)Nc2cccc([C@]3(c4nncn4C)C[C@@H](C)C3)c2)nc2cc[nH]c12]

### Step A: 7-chloro-1H-pyrrolo[3,2-b]pyridine 4-oxide

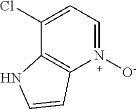

To a solution of 7-chloro-1H-pyrrolo[3,2-b]pyridine (5 g, 32.89 mmol) in DCM (100 mL) was added m-CPBA (9 g, 52.32 mmol) at 0° C. The reaction mixture was stirred for 12 h at 25° C. After completed, the mixture was filtered and the solid was washed with DCM (3×30 mL). The solid was purified by column chromatography on silica gel eluting with methanol in dichloromethane (10%) to afford the title compound (4.8 g, 87%). ¹H NMR (400 MHz, DMSO-d₆) δ 12.40 (s, 1H), 8.10 (d, J=6.6 Hz, 1H), 7.72 (s, 1H), 7.30 (d, J=6.6 Hz, 1H), 6.81 (d, J=2.8 Hz, 1H) ppm. MS: M/e 169, 171 (M+H)⁺.

### Step B: 7-chloro-1H-pyrrolo[3,2-b]pyridine-5-carboxylic acid

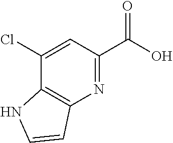 [SMILES: O=C(O)c1cc(Cl)c2[nH]ccc2n1]

A solution of 7-chloro-1H-pyrrolo[3,2-b]pyridine-5-carbonitrile (10 g, 56 mmol) and NaOH solution (10 M, 56 mL, 0.56 mol) in EtOH (112 mL) was heated at 100° C. in a glass sealed tube overnight. The reaction mixture was concentrated. The residue was dissolved in water (50 mL) and acidified to pH=5-6 with 5 N HCl solution. After concentrated, the crude product was slurried in MeOH (100 mL) for 2 hrs. The suspension was filtered and the filtrate was concentrated to get the product (11 g, 100%). MS: M/e 197 (M+1)+

### Step C: methyl 7-chloro-1H-pyrrolo[3,2-b]pyridine-5-carboxylate

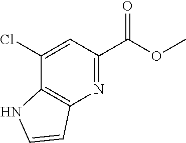

Concentrated H₂SO₄ (10 mL) was added to a solution of 7-chloro-1H-pyrrolo[3,2-b]pyridine-5-carboxylic acid (11 g, 56 mmol) in MeOH (100 mL) and it was heated at 90° C. overnight. The organic layer was dried, concentrated and purified by CombiFlash (DCM:MeOH=3%) to get the product (10 g, 85%). ¹H NMR (400 MHz, DMSO-d6) δ 12.30 (br. s, 1H), 7.92 (d, J=4.0 Hz, 1H), 7.91 (s, 1H), 6.82 (d, J=4.0 Hz, 1H), 3.90 (s, 3H). ppm. MS: M/e 211 (M+1)⁺

### Step D: methyl 7-vinyl-1H-pyrrolo[3,2-b]pyridine-5-carboxylate

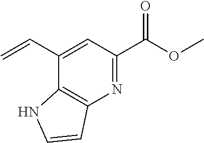

A solution of methyl 7-chloro-1H-pyrrolo[3,2-b]pyridine-5-carboxylate (5.4 g, 25.7 mmol), potassium trifluoro(vinyl)borate (6.9 g, 51.4 mmol), XPhosPdG2 (1 g, 1.3 mmol) and Cs₂CO₃ (16.7 g, 51.4 mmol) in dioxane (100 mL) and water (10 mL) was heated at 90° C. under N₂ balloon overnight. Then the solvent was evaporated. The residue was dissolved in DCM (20 mL), slurried and filtered. The filtrated was added with water (20 mL) and extracted with DCM (40 mL). The organic layer was dried, concentrated and purified by CombiFlash (DCM:MeOH=5%) to get the product (4 g, 77%). ¹H NMR (400 MHz, DMSO-d6) δ 11.91 (br. s, 1H), 8.01 (s, 1H), 7.86 (t, J=4.0 Hz, 1H), 7.28-7.21 (m, 1H), 6.73 (s, 1H), 6.27 (d, J=16.0 Hz, 1H), 5.72 (d, J=12.0 Hz, 1H), 3.89 (s, 3H). ppm. MS: M/e 203 (M+1)⁺

### Step E: lithium 7-vinyl-1H-pyrrolo[3,2-b]pyridine-5-carboxylate

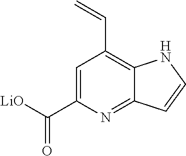

LiOH·H₂O solution (504 mg, 12 mmol, in 2 mL of water) was added to a solution of methyl 7-vinyl-1H-pyrrolo[3,2-b]pyridine-5-carboxylate (800 mg, 4 mmol) in MeOH (10 mL) and it was stirred at r.t overnight. The reaction mixture was evaporated, dissolved in water (3 mL) and lyophilized to get the product as a salt (740 mg, crude). MS: M/e 189 (M+1)⁺

### Step F: N-(3-((1s, 3s)-3-methyl-1-(4-methyl-4H-1,2,4-triazol-3-yl) cyclobutyl)phenyl)-7-vinyl-1H-pyrrolo[3,2-b]pyridine-5-carboxamide

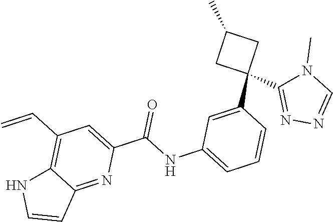 [SMILES: C=Cc1cc(C(=O)Nc2cccc([C@]3(c4nncn4C)C[C@@H](C)C3)c2)nc2cc[nH]c12]

A solution of the product of step D (740 mg, 4 mmol) and 3-((1s, 3s)-3-methyl-1-(4-methyl-4H-1,2,4-triazol-3-yl)cyclobutyl)aniline (968 mg, 4 mmol) in DMF (15 mL) were added with HATU (2.3 g, 6 mmol) and DIEA (1 g, 8 mmol). The reaction mixture was stirred at r.t for 2 hrs, then added with water (15 mL), extracted with DCM (20 mL) and washed with brine (20 mL). The organic layer was dried, concentrated and purified by CombiFlash (DCM:MeOH=8%) to get the product (900 mg, 56%). MS: M/e 413 (M+1)⁺

### Step G: 7-formyl-N-(3-((1s, 3s)-3-methyl-1-(4-methyl-4H-1,2,4-triazol-3-yl) cyclobutyl)phenyl)-1H-pyrrolo[3,2-b]pyridine-5-carboxamide

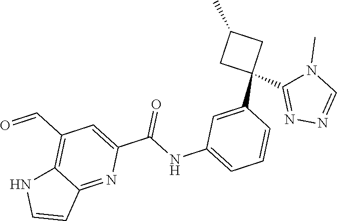 [SMILES: Cn1cnnc1[C@]1(c2cccc(NC(=O)c3cc(C=O)c4[nH]ccc4n3)c2)C[C@H](C)C1]

A solution of N-(3-((1s, 3s)-3-methyl-1-(4-methyl-4H-1,2,4-triazol-3-yl) cyclobutyl)phenyl)-7-vinyl-1H-pyrrolo[3,2-b]pyridine-5-carboxamide (900 mg, 2.2 mmol) and K₂Os₄·2H₂O (81 mg, 0.22 mmol) in dioxane (20 mL) and water (4 mL) was cooled under ice bath. NaIO₄ (1.4 g, 6.6 mmol) was added in portionwise. Then it was stirred at r.t for 2 hrs. The thickness suspension was filtered and washed with methanol (5 mL). The filtrate was concentrated to get the crude product, which was purified by CombiFlash (DCM:MeOH=5%) to get the pure product (520 mg, 58%). ¹H NMR (400 MHz, DMSO-d6) δ 12.23 (s, 1H), 10.69 (s, 1H), 10.40 (s, 1H), 8.49 (s, 1H), 8.28 (s, 1H), 7.96-7.91 (m, 3H), 7.34 (t, J=8.0 Hz, 1H), 6.97 (d, J=8.0 Hz, 1H), 6.90 (s, 1H), 3.18 (s, 3H), 2.83-2.80 (m, 2H), 2.59-2.51 (m, 3H), 1.07 (d, J=8.0 Hz, 3H). ppm. MS: M/e 415 (M+1)⁺

### Step H: 7-((isobutylamino)methyl)-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide

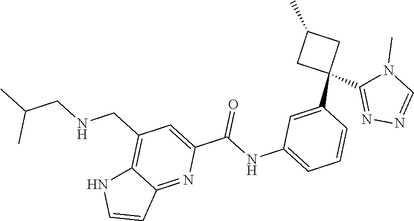 [SMILES: CC(C)CNCc1cc(C(=O)Nc2cccc([C@]3(c4nncn4C)C[C@@H](C)C3)c2)nc2cc[nH]c12]

A solution of 7-formyl-N-(3-((1s, 3s)-3-methyl-1-(4-methyl-4H-1,2,4-triazol-3-yl) cyclobutyl)phenyl)-1H-pyrrolo[3,2-b]pyridine-5-carboxamide (30 mg, 0.07 mmol) and 2-methylpropan-1-amine (11 mg, 0.14 mmol) in DCM (1 mL) was stirred at r.t for 30 mins. Then it was cooled under ice bath, and NaBH(OAc)₃ (30 mg, 0.14 mmol) was added. The reaction mixture was stirred at r.t for 6 hrs, more of NaBH(OAc)₃ (15 mg, 0.07 mmol) was added and it was stirred overnight. The reaction mixture was added with water (3 mL), basified with NaHCO₃ solution to pH=7˜8 and extracted with DCM (8 mL). The organic layer was dried, concentrated and purified by prep.HPLC to get the product (17 mg, 50%). ¹H NMR (400 MHz, CD3OD) δ 8.30 (s, 1H), 8.08 (s, 1H), 7.94 (s, 1H), 7.75 (s, 2H), 7.41 (d, J=8.0 Hz, 1H), 7.12 (t, J=8.0 Hz, 1H), 6.79 (s, 1H), 4.30 (s, 2H), 3.32 (s, 3H), 2.95 (s, 2H), 2.72-2.62 (m, 5H), 1.88-1.94 (m, 1H), 1.17 (d, J=4.0 Hz, 3H), 0.98 (d, J=8.0 Hz, 6H). ppm. MS: M/e 472 (M+1)+

### Compound B2: 7-((isobutylamino)methyl)-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

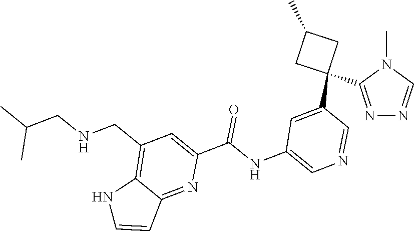 [SMILES: CC(C)CNCc1cc(C(=O)Nc2cncc([C@]3(c4nncn4C)C[C@@H](C)C3)c2)nc2cc[nH]c12]

### Step 1: 7-ethenyl-1H-pyrrolo[3,2-b]pyridine-5-carbonitrile

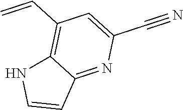

To a stirred solution of 7-chloro-1H-pyrrolo[3,2-b]pyridine-5-carbonitrile (1 g, 5.349 mmol, 1 equiv, 95%) and potassium ethenyltrifluoroboranuide (1.13 g, 8.024 mmol, 1.5 equiv, 95%) in dioxane (20 mL, 100%) and H₂O (2 mL, 100%) were added K₃PO₄ (2.39 g, 10.698 mmol, 2 equiv, 95%) and di-tert-butyl({dichloro[di-tert-butyl(phenyl)-lambda5-phosphanyl]palladio})phenyl-lambda5-phosphane (0.35 g, 0.535 mmol, 0.1 equiv, 95%) at room temperature. The resulting mixture was stirred for 4 h at 100° C. under nitrogen atmosphere. The mixture was allowed to cool down to room temperature. The resulting mixture was concentrated under vacuum. The residue was purified by silica gel column chromatography, eluted with CH₂Cl₂/MeOH (10:1) to afford 7-ethenyl-1H-pyrrolo[3,2-b]pyridine-5-carbonitrile (800 mg, 83.53%) as a yellow solid. LC-MS (M+H)⁺:=170.

### Step 2: 7-formyl-1H-pyrrolo[3,2-b]pyridine-5-carbonitrile

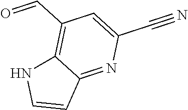 [SMILES: N#Cc1cc(C=O)c2[nH]ccc2n1]

To a stirred solution of 7-ethenyl-1H-pyrrolo[3,2-b]pyridine-5-carbonitrile (800 mg, 4.681 mmol, 1 equiv, 99.0%) in acetone (60 mL, 100%) and H₂O (12 mL, 100%) was added NMO (865.89 mg, 7.021 mmol, 1.5 equiv, 95%) and K₂OsO₄·2H₂O (181.56 mg, 0.468 mmol, 0.1 equiv, 95%) at room temperature. The resulting mixture was stirred for 2 h at room temperature. To the above mixture was added NaIO₄ (3161.89 mg, 14.043 mmol, 3 equiv, 95%) at room temperature. The resulting mixture was stirred for additional overnight at room temperature. The resulting mixture was diluted with MeOH (10 mL). The resulting mixture was filtered, the filter cake was washed with MeOH (2×2 mL). The filtrate was concentrated under reduced pressure. The residue was purified by silica gel column chromatography, eluted with PE/EA (1:1) to afford 7-formyl-1H-pyrrolo[3,2-b]pyridine-5-carbonitrile (500 mg, 57.41%) as a yellow solid. LC-MS (M+H)⁺:=172.

### Step 3: 7-{[(2-methylpropyl)amino]methyl}-1H-pyrrolo[3,2-b]pyridine-5-carbonitrile

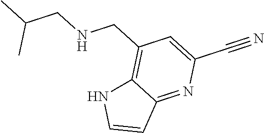 [SMILES: CC(C)CNCc1cc(C#N)nc2cc[nH]c12]

To a stirred solution of 7-formyl-1H-pyrrolo[3,2-b]pyridine-5-carbonitrile (100 mg, 0.538 mmol, 1 equiv, 92.0%) and isobutylamine (82.76 mg, 1.076 mmol, 2 equiv, 95%) in MeOH (3 mL, 100%) were added AcOH (67.96 mg, 1.076 mmol, 2 equiv, 95%) at room temperature. The resulting mixture was stirred for 1 h at room temperature. To the above mixture was added NaBH₃CN (71.11 mg, 1.076 mmol, 2 equiv, 95%) at room temperature. The resulting mixture was stirred for additional 2 h at room temperature. The resulting mixture was concentrated under reduced pressure. The residue was purified by silica gel column chromatography, eluted with PE/EA (1:1) to afford 7-{[(2-methylpropyl)amino]methyl}-1H-pyrrolo[3,2-b]pyridine-5-carbonitrile (80 mg, 58.67%) as a yellow solid. LC-MS (M+H)⁺:=229.

### Step 4: 7-{[(2-methylpropyl)amino]methyl}-1H-pyrrolo[3,2-b]pyridine-5-carboxylic acid

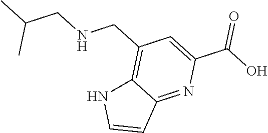 [SMILES: CC(C)CNCc1cc(C(=O)O)nc2cc[nH]c12]

A solution of 7-{[(2-methylpropyl)amino]methyl}-1H-pyrrolo[3,2-b]pyridine-5-carbonitrile (80 mg, 0.315 mmol, 1 equiv, 90%) in H₂SO₄ (3 mL, 33.772 mmol, 107.08 equiv, 60%) was stirred for 1 h at 100° C. The mixture was allowed to cool down to 0° C. The mixture was neutralized to pH 3 with NaOH (2N). The resulting mixture was concentrated under vacuum. The resulting mixture was diluted with MeOH (5 mL). The resulting mixture was filtered, the filter cake was washed with MeOH (3×5 mL). The filtrate was concentrated under reduced pressure. The residue was purified by reverse flash chromatography with the following conditions: column, C18 silica gel; mobile phase, MeCN in Water (0.1% TFA), 0% to 10% gradient in 10 min; detector, UV 220 m. This resulted in 7-{[(2-methylpropyl)amino]methyl}-1H-pyrrolo[3,2-b]pyridine-5-carboxylic acid (50 mg, 57.70%) as a yellow solid. LC-MS (M+H)⁺:=248.

### Step 5: 7-{[(2-methylpropyl)amino]methyl}-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide

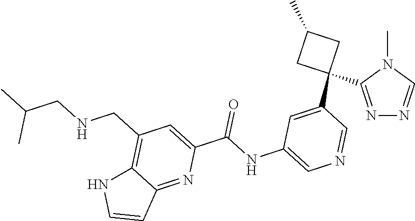 [SMILES: CC(C)CNCc1cc(C(=O)Nc2cncc([C@]3(c4nncn4C)C[C@@H](C)C3)c2)nc2cc[nH]c12]

To a stirred solution of 7-{[(2-methylpropyl)amino]methyl}-1H-pyrrolo[3,2-b]pyridine-5-carboxylic acid (20 mg, 0.073 mmol, 1 equiv, 90%) and 5-[(1r, 3s)-3-methyl-1-(4-methyl-1,2,4-triazol-3-yl)cyclobutyl]pyridin-3-amine (18.64 mg, 0.073 mmol, 1 equiv, 95%) in Pyridine (3 mL, 100%) was added T₃P (73.13 mg, 0.219 mmol, 3 equiv, 95%) at room temperature. The resulting mixture was stirred for 3 h at room temperature. The resulting mixture was concentrated under vacuum. The crude product was purified by Chiral-Prep-HPLC with the following conditions (2 #SHIMADZU (HPLC-01)): Column, XBridge Prep OBD C18 Column, 30*150 mm, 5 μm; mobile phase, 10 mmol NH₄HCO₃+0.05% NH₃H₂O and ACN (25% ACN up to 55% in 8 min); Detector, UV product was obtained which Alpha. This resulted in 7-{[(2-methylpropyl)amino]methyl}-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide (12.1 mg, 34.09%). LC-MS (M+H)⁺:=473. ¹H NMR (300 MHz, DMSO-d₆) δ 10.81 (s, 1H), 9.11 (d, J=2.2 Hz, 1H), 8.33 (s, 2H), 8.23 (d, J=2.1 Hz, 1H), 8.04 (s, 1H), 7.79 (d, J=3.3 Hz, 1H), 6.74 (d, J=3.2 Hz, 1H), 4.09 (s, 2H), 3.23 (s, 3H), 2.88 (q, J=6.1 Hz, 2H), 2.59 (d, J=7.6 Hz, 3H), 2.35 (d, J=6.7 Hz, 2H), 1.72 (dq, J=13.3, 6.7 Hz, 1H), 1.10 (d, J=5.4 Hz, 3H), 0.89 (d, J=6.6 Hz, 6H) ppm.

### Compound B3: 7-((((S)-1-cyclopropylethyl)amino)methyl)-N-(3-((1s,3R)-3-methyl-1-(4-methyl-4H-1,2,4-triazol-3-yl)cyclobutyl)phenyl)-1H-pyrrolo[3,2-b]pyridine-5-carboxamide

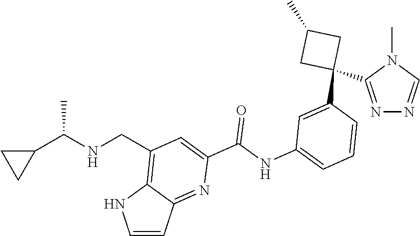 [SMILES: C[C@H](NCc1cc(C(=O)Nc2cccc([C@]3(c4nncn4C)C[C@@H](C)C3)c2)nc2cc[nH]c12)C1CC1]

A solution of 7-formyl-N-(3-((1s, 3s)-3-methyl-1-(4-methyl-4H-1,2,4-triazol-3-yl) cyclobutyl)phenyl)-1H-pyrrolo[3,2-b]pyridine-5-carboxamide (30 mg, 0.07 mmol) and (S)-1-cyclopropylethan-1-amine hydrochloride (17 mg, 0.14 mmol) in DCM (1 mL) was stirred at r.t for 30 mins. Then it was cooled under ice bath, and NaBH(OAc)₃ (30 mg, 0.14 mmol) was added. The reaction mixture was stirred at r.t for 6 hrs, more of NaBH(OAc)₃ (15 mg, 0.07 mmol) was added and it was stirred overnight. The reaction mixture was added with 43 water (3 mL), basified with NaHCO₃ solution to pH=7-8 and extracted with DCM (8 mL). The organic layer was dried, concentrated and purified by prep. HPLC (FA) to get the product (18 mg, 51%). ¹H NMR (400 MHz, DMSO-d6) δ 10.57 (s, 1H), 8.29 (s, 1H), 8.04 (s, 1H), 7.95 (s, 1H), 7.91 (d, J=4.0 Hz, 1H), 7.80 (t, J=4.0 Hz, 1H), 7.34 (t, J=4.0 Hz, 1H), 6.95 (d, J=4.0 Hz, 1H), 6.74 (d, J=4.0 Hz, 1H), 4.23-1.16 (q, 2H), 3.20 (s, 3H), 2.84 (d, J=8.0 Hz, 2H), 2.64-2.53 (m, 4H), 1.95-1.89 (m, 1H), 1.14 (d, J=4.0 Hz, 3H), 1.10 (d, J=4.0 Hz, 3H), 0.76-0.71 (m, 1H), 0.47-0.41 (m, 1H), 0.37-0.32 (m, 1H), 0.20-0.15 (m, 1H), 0.04-0.01 (m, 1H). ppm. MS: M/e 484 (M+1)⁺

### Compound B4: N-(3-((1s,3R)-3-(cyanomethyl)-1-(4-methyl-4H-1,2,4-triazol-3-yl)cyclobutyl)phenyl)-7-(((S)-3-methylpiperidin-1-yl)methyl)-1H-pyrrolo[3,2-b]pyridine-5-carboxamide

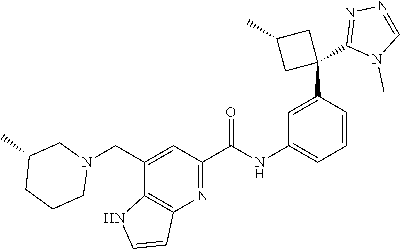 [SMILES: C[C@H]1CCCN(Cc2cc(C(=O)Nc3cccc([C@]4(c5nncn5C)C[C@@H](C)C4)c3)nc3cc[nH]c23)C1]

### Step A: 7-chloro-1H-pyrrolo[3,2-b]pyridine 4-oxide

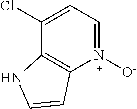

To a solution of 7-chloro-1H-pyrrolo[3,2-b]pyridine (5 g, 32.89 mmol) in DCM (100 mL) was added m-CPBA (9 g, 52.32 mmol) at 0° C. The reaction mixture was stirred for 12 h at 25° C. After completed, the mixture was filtered and the solid was washed with DCM (3×30 mL). The solid was purified by column chromatography on silica gel eluting with methanol in dichloromethane (10%) to afford the title compound (4.8 g, 87%). ¹H NMR (400 MHz, DMSO-d₆) δ 12.40 (s, 1H), 8.10 (d, J=6.6 Hz, 1H), 7.72 (s, 1H), 7.30 (d, J=6.6 Hz, 1H), 6.81 (d, J=2.8 Hz, 1H) ppm. MS: M/e 169, 171 (M+H)⁺.

### Step B: 7-chloro-1H-pyrrolo[3,2-b]pyridine-5-carbonitrile

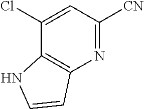 [SMILES: N#Cc1cc(Cl)c2[nH]ccc2n1]

To a solution of 7-chloro-1H-pyrrolo[3,2-b]pyridine 4-oxide (5.3 g, 31.55 mmol), Et₃N (4.8 g, 47.52 mmol) in ACN (150 mL) was added TMSCN (15.6 g, 157.57 mmol) at 0° C. under N₂. The reaction mixture was stirred for 60 h at 85° C. After completed, the solvent was removed by in vacuo. The mixture was diluted with water (50 mL) and DCM (50 mL) and extracted with DCM/MeOH (10/1, 3×200 mL), dried over Na₂SO₄ and concentrated under vacuum. The crude title compound (5.6 g) was obtained. ¹H NMR (400 MHz, DMSO-d₆) δ 12.51 (s, 1H), 8.02 (d, J=3.2 Hz, 1H), 7.99 (s, 1H), 6.84 (d, J=3.2 Hz, 1H) ppm. MS: M/e 178, 180 (M+H)⁺.

### Step C: tert-butyl 7-chloro-5-cyano-1H-pyrrolo[3,2-b]pyridine-1-carboxylate

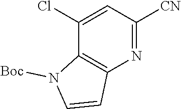 [SMILES: CC(C)(C)OC(=O)n1ccc2nc(C#N)cc(Cl)c21]

To a solution of 7-chloro-1H-pyrrolo[3,2-b]pyridine-5-carbonitrile (5.6 g, impure), Et₃N (4.8 g, 47.52 mmol) and DMAP (380 mg, 3.11 mmol) in DCM (10 mL) was added Boc₂O (7.6 g, 34.86 mmol) at 0° C. The reaction mixture was stirred for 12 h at 25° C. After completed, the reaction was quenched with water (50 mL) and extracted with DCM (3×200 mL), dried over Na₂SO₄ and concentrated under vacuum. The residue was purified by column chromatography on silica gel eluting with ethyl acetate in petroleum ether (30%) to afford the title compound (7.8 g, 89%). ¹H NMR (400 MHz, DMSO-d₆) 8.30 (d, J=3.6 Hz, 1H), 8.19 (s, 1H), 7.02 (d, J=3.6 Hz, 1H), 1.63 (s, 9H) ppm. MS: M/e 278, 280 (M+H)⁺.

### Step D: 7-iodo-1H-pyrrolo[3,2-b]pyridine-5-carbonitrile

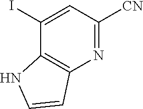 [SMILES: N#Cc1cc(I)c2[nH]ccc2n1]

To a solution of tert-butyl 7-chloro-5-cyano-1H-pyrrolo[3,2-b]pyridine-1-carboxylate (500 mg, 1.78 mmol), NaI (1.3 g, 8.66 mmol) in ACN (15 mL) was added AcCl (280 mg, 3.59 mmol) at 0° C. under N₂. The reaction mixture was stirred for 2 h at 80° C. After completed, the reaction was quenched with aq NaHCO₃ (20 mL) and extracted with EtOAc (3×30 mL), dried over Na₂SO₄ and concentrated under vacuum. The residue was purified by column chromatography on silica gel eluting with methanol in dichloromethane (6%) to afford the title compound (280 mg, 43%). MS: M/e 270 (M+H)⁺.

### Step E: 7-iodo-1-((2-(trimethylsilyl)ethoxy)methyl)-1H-pyrrolo[3,2-b]pyridine-5-carbonitrile

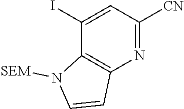 [SMILES: C[Si](C)(C)CCOCn1ccc2nc(C#N)cc(I)c21]

To a solution of 7-iodo-1H-pyrrolo[3,2-b]pyridine-5-carbonitrile (280 mg, 1.04 mmol) in THF (10 mL) was added NaH (50 mg, 1.25 mmol) at 0° C. After stirring for 0.5 h at 0° C., SEMCl (226 mg, 1.35 mmol) was added dropwise. The reaction mixture was stirred for 12 h at 25° C. After completed, the reaction was quenched with aq NH₄Cl (20 mL) and extracted with DCM (3×50 mL), dried over Na₂SO₄ and concentrated under vacuum. The residue was purified by column chromatography on silica gel eluting with ethyl acetate in petroleum ether (20%) to afford the title compound (290 mg, crude). MS: M/e 400 (M+H)⁺.

### Step F: 7-(hydroxymethyl)-1-((2-(trimethylsilyl)ethoxy)methyl)-1H-pyrrolo[3,2-b]pyridine-5-carbonitrile

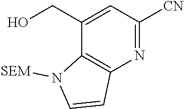 [SMILES: C[Si](C)(C)CCOCn1ccc2nc(C#N)cc(CO)c21]

To a solution of 7-iodo-1-((2-(trimethylsilyl)ethoxy)methyl)-1H-pyrrolo[3,2-b]pyridine-5-carbonitrile (150 mg, 0.376 mmol), (tributylstannyl)methanol (370 mg, 1.15 mmol) in PhMe (5 mL) was added PdCl₂(PPh₃)₂(30 mg, 0.043 mmol) and KF (131 mg, 2.26 mmol) under N₂. The reaction mixture was stirred for 12 h at 105° C. After completed, the solvent was removed by in vacuo. The residue was purified by column chromatography on silica gel eluting with ethyl acetate in petroleum ether (50%) to afford the title compound (40 mg, 35%). ¹H NMR (400 MHz, DMSO-d₆) 8.05 (d, J=3.2 Hz, 1H), 7.74 (s, 1H), 6.76 (d, J=3.2 Hz, 1H), 5.72 (t, J=5.6 Hz, 1H), 5.66 (s, 2H), 5.02 (d, J=5.6 Hz, 2H), 3.49-3.41 (m, 2H), 0.83-0.77 (m, 2H), −0.10 (s, 9H). MS: M/e 304 (M+H)⁺.

### Step G: 7-formyl-1-((2-(trimethylsilyl)ethoxy)methyl)-1H-pyrrolo[3,2-b]pyridine-5-carbonitrile

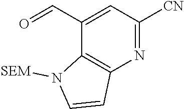 [SMILES: C[Si](C)(C)CCOCn1ccc2nc(C#N)cc(C=O)c21]

To a solution of 7-(hydroxymethyl)-1-((2-(trimethylsilyl)ethoxy)methyl)-1H-pyrrolo[3,2-b]pyridine-5-carbonitrile (30 mg, 1.78 mmol) in DCM (15 mL) was added Dess-Martin Periodinane (63 mg, 0.148 mmol) at 0° C. The reaction mixture was stirred for 2 h at 25° C. After completed, the mixture was filtered and the filtrate was collected and concentrated under vacuum. The residue was purified by Prep-TLC (PE/EtOAc=4/1) to afford the title compound (40 mg, impure). MS: M/e 302 (M+H)⁺.

### Step H: (S)-7-((3-methylpiperidin-1-yl)methyl)-1-((2-(trimethylsilyl)ethoxy)methyl)-1H-pyrrolo[3,2-b]pyridine-5-carbonitrile

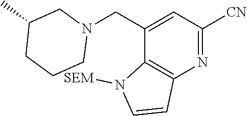 [SMILES: C[C@H]1CCCN(Cc2cc(C#N)nc3ccn(COCC[Si](C)(C)C)c23)C1]

To a solution of 7-formyl-1-((2-(trimethylsilyl)ethoxy)methyl)-1H-pyrrolo[3,2-b]pyridine-5-carbonitrile (40 mg, impure), (S)-3-methylpiperidine hydrochloride (27 mg, 0.198 mmol), Et₃N (27 mg, 0.267 mmol) in DCM (3 mL) was added NaBH(OAc)₃ (56 mg, 0.264 mmol). The reaction mixture was stirred for 12 h at 25° C. After completed, the reaction was quenched with aq NaHCO₃ (20 mL) and extracted with DCM (3×20 mL), dried over Na₂SO₄ and concentrated under vacuum. The residue was purified by Prep-TLC (PE/EtOAc=3/1) to afford the title compound (40 mg, 79%). MS: M/e 385 (M+H)⁺.

### Step I: (S)-7-((3-methylpiperidin-1-yl)methyl)-1-((2-(trimethylsilyl)ethoxy)methyl)-1H-pyrrolo[3,2-b]pyridine-5-carboxamide

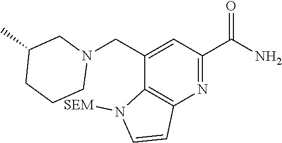 [SMILES: C[C@H]1CCCN(Cc2cc(C(N)=O)nc3ccn(COCC[Si](C)(C)C)c23)C1]

To a solution of (S)-7-((3-methylpiperidin-1-yl)methyl)-1-((2-(trimethylsilyl)ethoxy)methyl)-1H-pyrrolo[3,2-b]pyridine-5-carbonitrile (40 mg, 0.104 mmol) in EtOH (5 mL)/H₂O (2 mL) was added NaOH (21 mg, 0.525 mmol). The reaction mixture was stirred for 12 h at 90° C. After completed, the solvent was removed by in vacuo. The crude title compound (45 mg, impure) was obtained. MS: M/e 403 (M+H)⁺.

### Step J: N-(3-((1s,3R)-3-methyl-1-(4-methyl-4H-1,2,4-triazol-3-yl)cyclobutyl)phenyl)-7-(((S)-3-methylpiperidin-1-yl)methyl)-1-((2-(trimethylsilyl)ethoxy)methyl)-1H-pyrrolo[3,2-b]pyridine-5-carboxamide

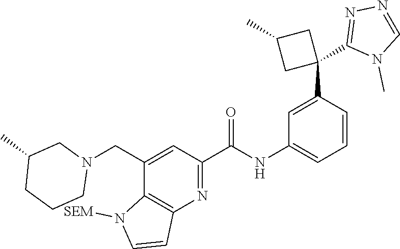 [SMILES: C[C@H]1CCCN(Cc2cc(C(=O)Nc3cccc([C@]4(c5nncn5C)C[C@@H](C)C4)c3)nc3ccn(COCC[Si](C)(C)C)c23)C1]

To a solution of (S)-7-((3-methylpiperidin-1-yl)methyl)-1-((2-(trimethylsilyl)ethoxy)methyl)-1H-pyrrolo[3,2-b]pyridine-5-carboxamide (45 mg, impure), 3-((1s, 3s)-1-(3-bromophenyl)-3-methylcyclobutyl)-4-methyl-4H-1,2,4-triazole (38 mg, 0.124 mmol) in dioxane (1 mL) were added t-BuXphos G3 Pd (9 mg, 0.011 mmol), t-BuXphos (5 mg, 0.011 mmol) and t-BuONa (20 mg, 0.208 mmol) under N₂. The reaction mixture was stirred for 12 h at 100° C. After completed, the reaction was quenched with aq NH₄Cl (10 mL) and extracted with EtOAc (3×30 mL), dried over Na₂SO₄ and concentrated under vacuum. The residue was purified by Prep-TLC (DCM/MeOH=15/1) to afford the title compound (30 mg, impure). MS: M/e 628 (M+H)⁺.

### Step K: N-(3-((1s,3R)-3-methyl-1-(4-methyl-4H-1,2,4-triazol-3-yl)cyclobutyl)phenyl)-7-(((S)-3-methylpiperidin-1-yl)methyl)-1H-pyrrolo[3,2-b]pyridine-5-carboxamide

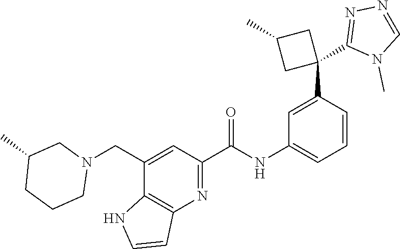 [SMILES: C[C@H]1CCCN(Cc2cc(C(=O)Nc3cccc([C@]4(c5nncn5C)C[C@@H](C)C4)c3)nc3cc[nH]c23)C1]

To a solution of N-(3-((1s,3R)-3-methyl-1-(4-methyl-4H-1,2,4-triazol-3-yl)cyclobutyl)phenyl)-7-(((S)-3-methylpiperidin-1-yl)methyl)-1-((2-(trimethylsilyl) ethoxy)methyl)-1H-pyrrolo[3,2-b]pyridine-5-carboxamide (30 mg, impure) in DCM (5 mL) was added TFA (1 mL). The reaction mixture was stirred for 24 h at 25° C. After completed, the solvent was removed by in vacuo. The residue was dissolved in MeOH (5 mL) and Ethylenediamine (60 uL) was added. The reaction mixture was stirred for 12 h at 25° C. After completed, the solvent was removed by in vacuo. The residue was purified by prep-HPLC to afford the title compound (2 mg, 8%). ¹H NMR (400 MHz, CD₃OD) δ 8.30 (s, 1H), 8.00 (s, 1H), 7.94 (s, 1H), 7.75 (d, J=8.4 Hz, 1H), 7.72 (d, J=3.2 Hz, 1H), 7.40 (t, J=8.0 Hz, 1H), 7.10 (d, J=7.4 Hz, 1H), 6.77 (d, J=2.8 Hz, 1H), 3.99 (s, 2H), 3.00-2.88 (m, 4H), 2.87-2.53 (m, 6H), 2.19-2.10 (m, 1H), 1.89-1.65 (m, 5H), 1.16 (d, J=6.4 Hz, 3H), 1.03-0.92 (m, 1H), 0.87 (d, J=6.0 Hz, 3H) ppm. MS: M/e 498 (M+H)⁺.

### Compound B5: N-(3-((1s,3R)-3-(cyanomethyl)-1-(4-methyl-4H-1,2,4-triazol-3-yl)cyclobutyl)phenyl)-7-(((S)-3-methylpiperidin-1-yl)methyl)-1H-pyrrolo[3,2-b]pyridine-5-carboxamide

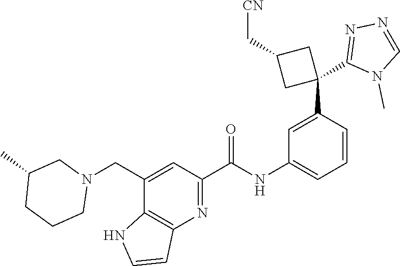 [SMILES: C[C@H]1CCCN(Cc2cc(C(=O)Nc3cccc([C@]4(c5nncn5C)C[C@@H](CC#N)C4)c3)nc3cc[nH]c23)C1]

### Step A: 7-chloro-1H-pyrrolo[3,2-b]pyridine-5-carboxylic acid

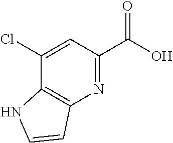 [SMILES: O=C(O)c1cc(Cl)c2[nH]ccc2n1]

To a solution of 7-chloro-1H-pyrrolo[3,2-b]pyridine-5-carbonitrile (10 g, impure) in EtOH (80 mL) was added NaOH (50 mL, 500.0 mmol, 10 N). The reaction mixture was stirred for 12 h at 120° C. in seal tube. After completed, the solvent was removed by in vacuo. the residue was diluted with MeOH (100 mL) and acidized with con H₂SO₄ to adjust PH=5-6. The mixture was filtered and the filate was collected and concentrated under vacuum. The crude title compound (12 g) was obtained. MS: M/e 197, 199 (M+H)⁺.

### Step B: methyl 7-chloro-1H-pyrrolo[3,2-b]pyridine-5-carboxylate

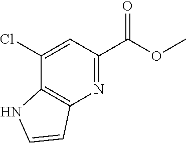

To a solution of 7-chloro-1H-pyrrolo[3,2-b]pyridine-5-carboxylic acid (12 g, impure) in MeOH (150 mL) was added con H₂SO₄ (10 mL). The reaction mixture was stirred for 48 h at 80° C. After completed, the mixture was based with K₂CO₃ and Na₂CO₃ to adjust PH=8. The mixture was filtered and the filate was collected and concentrated under vacuum. The residue was diluted with water (100 mL) and extracted with EtOAc (3×100 mL), dried over Na₂SO₄ and concentrated under vacuum. The crude title compound (8 g) was obtained. MS: M/e 211, 213 (M+H)⁺.

### Step C: methyl 7-chloro-1-((2-(trimethylsilyl)ethoxy)methyl)-1H-pyrrolo[3,2-b]pyridine-5-carboxylate

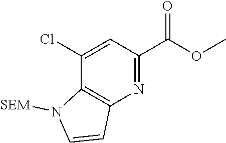 [SMILES: COC(=O)c1cc(Cl)c2c(ccn2COCC[Si](C)(C)C)n1]

To a solution of methyl 7-chloro-1H-pyrrolo[3,2-b]pyridine-5-carboxylate (7 g, 33.1 mmol) in THF (150 mL) was added NaH (1.6 g, 40.0 mmol) at 0° C. After stirring for 10 min at 0° C., SEMCl (8.3 g, 49.7 mmol) was added dropwise. The reaction mixture was stirred for 12 h at 25° C. After completed, the reaction was quenched with aq NH₄Cl (50 mL) and extracted with DCM (3×80 mL), dried over Na₂SO₄ and concentrated under vacuum. The residue was purified by column chromatography on silica gel eluting with ethyl acetate in petroleum ether (40%) to afford the title compound (8 g, 71%). ¹H NMR (400 MHz, CD₃OD) δ 8.03 (s, 1H), 7.89 (d, J=3.2 Hz, 1H), 6.83 (d, J=3.2 Hz, 1H), 5.85 (s, 2H), 4.00 (s, 3H), 3.57 (t, J=8.0 Hz, 2H), 0.86 (t, J=8.0 Hz, 2H), −0.09 (s, 9H). MS: M/e 341, 343 (M+H)⁺.

### Step D: methyl 1-((2-(trimethylsilyl)ethoxy)methyl)-7-vinyl-1H-pyrrolo[3,2-b]pyridine-5-carboxylate

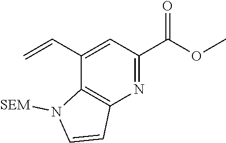 [SMILES: C=Cc1cc(C(=O)OC)nc2ccn(COCC[Si](C)(C)C)c12]

To a solution of methyl 7-chloro-1-((2-(trimethylsilyl)ethoxy)methyl)-1H-pyrrolo[3,2-b]pyridine-5-carboxylate (8 g, 23.46 mmol), potassium trifluoro(vinyl)borate (4.7 g, 35.07 mmol) in Dioxane (100 mL)/H₂O (10 mL) was added Xphos G2 Pd (930 mg, 1.18 mmol) and Cs₂CO₃ (15.3 g, 46.93 mmol) under N₂. The reaction mixture was stirred for 2 h at 80° C. After completed, the mixture was filtered and the filate was collected and concentrated under vacuum. The residue was purified by column chromatography on silica gel eluting with ethyl acetate in petroleum ether (40%) to afford the title compound (7.4 g, 95%). ¹H NMR (400 MHz, CD₃OD) δ 8.08 (s, 1H), 7.80 (d, J=3.2 Hz, 1H), 7.63 (dd, J=17.4, 11.0 Hz, 1H), 6.75 (d, J=3.2 Hz, 1H), 6.04 (d, J=17.3 Hz, 1H), 5.67 (d, J=11.1 Hz, 1H), 5.63 (s, 2H), 4.00 (s, 3H), 3.58 (t, J=8.0 Hz, 2H), 0.88 (t, J=8.0 Hz, 2H), −0.07 (s, 9H). MS: M/e 333 (M+H)⁺.

### Step E: 1-(tert-butyl) 5-methyl 7-formyl-1H-pyrrolo[3,2-b]pyridine-1,5-dicarboxylate

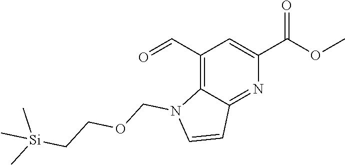

To a solution of methyl 1-((2-(trimethylsilyl)ethoxy)methyl)-7-vinyl-2,3-dihydro-1H-pyrrolo[3,2-b]pyridine-5-carboxylate (3.2 g, 9.6 mmol) in dioxane (30 mL)/H₂O (6 mL) was added K₂OsO₄ 2H₂O (352 mg, 960 umol) and NaIO₄ (8 g, 30.7 umol) one portion. The reaction was stirred at 15° C. for 16 h. The reaction was poured into H₂O (100 mL) extracted with EtOAc (20 mL*3). The combined organic phase was concentrated to give the crude product. The crude was purified by column chromatography (SiO₂, Petroleum ether/EtOAc=50:1 to 10:1) to give the product (1.8 g, 56% yield) as a yellow solid. MS(ESI) m/e [M+1]335; ¹H NMR (400 MHz, DMSO-d₆) δ=10.57 (s, 1H), 8.30 (s, 1H), 8.17 (d, J=4.0 Hz, 1H), 6.94 (d, J=4.0 Hz, 1H), 5.88 (s, 2H), 3.93 (s, 3H), 3.42 (t, J=8.0 Hz, 2H), 0.77 (t, J=8.0 Hz, 2H), -0.15 (s, 9H) ppm.

### Step F: methyl (S)-7-((3-methylpiperidin-1-yl)methyl)-1-((2-(trimethylsilyl)ethoxy) methyl)-1H-pyrrolo[3,2-b]pyridine-5-carboxylate

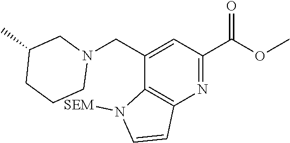

To a solution of 1-(tert-butyl) 5-methyl 7-formyl-1H-pyrrolo[3,2-b]pyridine-1,5-dicarboxylate (400 mg, 1.19 mmol), (S)-3-methylpiperidine hydrochloride (245 mg, 1.8 mmol), Et₃N (180 mg, 1.78 mmol) in DCM (8 mL) was added NaBH(OAc)₃ (504 mg, 2.37 mmol). The reaction mixture was stirred for 12 h at 25° C. After completed, the reaction was quenched with aq NaHCO₃ (20 mL) and extracted with DCM (3×50 mL), dried over Na₂SO₄ and concentrated under vacuum. The residue was purified by column chromatography on silica gel eluting with methanol in dichloromethane (5%) to afford the title compound (380 mg, impure). MS: M/e 418 (M+H)⁺.

### Step G: (S)-7-((3-methylpiperidin-1-yl)methyl)-1-((2-(trimethylsilyl)ethoxy)methyl)-1H-pyrrolo[3,2-b]pyridine-5-carboxylic acid

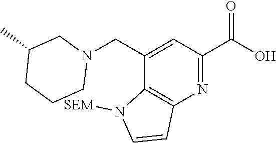 [SMILES: C[C@H]1CCCN(Cc2cc(C(=O)O)nc3ccn(COCC[Si](C)(C)C)c23)C1]

To a solution of methyl (S)-7-((3-methylpiperidin-1-yl)methyl)-1-((2-(trimethylsilyl)ethoxy)methyl)-1H-pyrrolo[3,2-b]pyridine-5-carboxylate (100 mg, 0.239 mmol) in MeOH (5 mL)/H₂O (0.5 mL) was added LiOH H₂O (50 mg, 1.19 mmol). The reaction mixture was stirred for 12 h at 25° C. After completed, the solvent was concentrated under vacuum. The residue was diluted with water and neutralized with 2 N HCl and freed dried to afford the title compound (100 mg, crude). MS: M/e 404 (M+H)⁺

### Step H: N-(3-((1s,3R)-3-(cyanomethyl)-1-(4-methyl-4H-1,2,4-triazol-3-yl)cyclobutyl) phenyl)-7-(((S)-3-methylpiperidin-1-yl)methyl)-1-((2-(trimethylsilyl)ethoxy)methyl)-1H-pyrrolo[3,2-b]pyridine-5-carboxamide

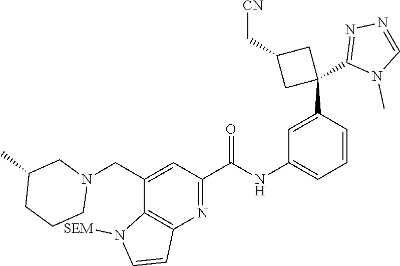 [SMILES: C[C@H]1CCCN(Cc2cc(C(=O)Nc3cccc([C@]4(c5nncn5C)C[C@@H](CC#N)C4)c3)nc3ccn(COCC[Si](C)(C)C)c23)C1]

To a solution of (S)-7-((3-methylpiperidin-1-yl)methyl)-1-((2-(trimethylsilyl)ethoxy)methyl)-1H-pyrrolo[3,2-b]pyridine-5-carboxylic acid (100 mg, crude), 2-((1s, 3s)-3-(3-aminophenyl)-3-(4-methyl-4H-1,2,4-triazol-3-yl)cyclobutyl)acetonitrile (70 mg, 0.262 mmol), DIEA (62 mg, 0.480 mmol) in DMF (3 mL) was added HATU (140 mg, 0.368 mmol). The reaction mixture was stirred for 1 h at 25° C. After completed, the reaction was quenched with aq NH₄Cl (10 mL) and extracted with DCM (3×20 mL), dried over Na₂SO₄ and concentrated under vacuum. The residue was purified by Prep-TLC (DCM/MeOH=20/1) to afford the title compound (150 mg, impure). MS: M/e 653 (M+H)⁺.

### Step I: N-(3-((1s,3R)-3-(cyanomethyl)-1-(4-methyl-4H-1,2,4-triazol-3-yl)cyclobutyl) phenyl)-7-(((S)-3-methylpiperidin-1-yl)methyl)-1H-pyrrolo[3,2-b]pyridine-5-carboxamide

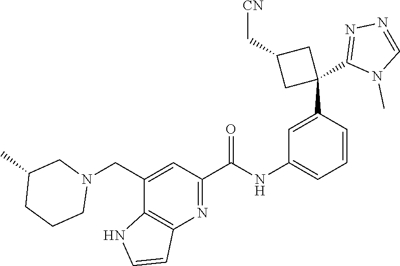 [SMILES: C[C@H]1CCCN(Cc2cc(C(=O)Nc3cccc([C@]4(c5nncn5C)C[C@@H](CC#N)C4)c3)nc3cc[nH]c23)C1]

To a solution of N-(3-((1s,3R)-3-(cyanomethyl)-1-(4-methyl-4H-1,2,4-triazol-3-yl)cyclobutyl) phenyl)-7-(((S)-3-methylpiperidin-1-yl)methyl)-1-((2-(trimethylsilyl)ethoxy)methyl)-1H-pyrrolo[3,2-b]pyridine-5-carboxamide (150 mg, impure) in DCM (2 mL) was added TFA (6 mL). The reaction mixture was stirred for 24 h at 25° C. After completed, the solvent was removed by in vacuo. The residue was dissolved in MeOH (5 mL) and Ethylenediamine (0.2 mL) was added. The reaction mixture was stirred for 24 h at 25° C. After completed, the solvent was removed by in vacuo. The mixture was diluted with water (10 mL) and extracted with DCM (3×50 mL), dried over Na₂SO₄ and concentrated under vacuum. The residue was purified by prep-HPLC to afford the title compound (50 mg, 42%); ¹H NMR (400 MHz, CD₃OD) δ 8.33 (s, 1H), 7.98 (s, 2H), 7.79 (d, J=8.0 Hz, 1H), 7.69 (d, J=3.2 Hz, 1H), 7.42 (t, J=8.0 Hz, 1H), 7.10 (d, J=7.6 Hz, 1H), 6.75 (d, J=3.2 Hz, 1H), 3.88 (s, 2H), 3.33 (s, 3H), 3.08-3.00 (m, 2H), 3.00-2.90 (m, 1H), 2.89-2.81 (m, 4H), 2.67 (d, J=6.0 Hz, 2H), 2.07-1.96 (m, 1H), 1.80-1.69 (m, 3H), 1.69-1.62 (m, 2H), 0.99-0.88 (m, 1H), 0.85 (d, J=5.6 Hz, 3H) ppm; MS: M/e 523 (M+H)⁺.

### Compound B6: (S)—N-(3-(3-((4-methyl-4H-1,2,4-triazol-3-yl)methyl)oxetan-3-yl)phenyl)-7-((3-methylpiperidin-1-yl)methyl)-1H-pyrrolo[3,2-b]pyridine-5-carboxamide

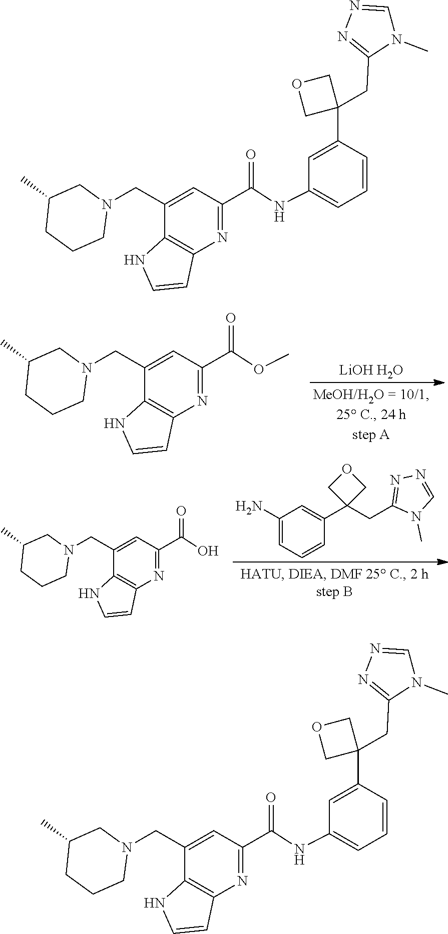

### Step A: (S)-7-((3-methylpiperidin-1-yl)methyl)-1H-pyrrolo[3,2-b]pyridine-5-carboxylic acid

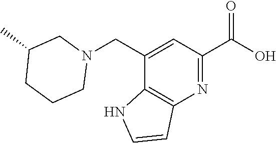 [SMILES: C[C@H]1CCCN(Cc2cc(C(=O)O)nc3cc[nH]c23)C1]

To a solution of methyl (S)-7-((3-methylpiperidin-1-yl)methyl)-1H-pyrrolo[3,2-b]pyridine-5-carboxylate (40 mg, 0.139 mmol) in MeOH (5 mL)/H₂O (0.5 mL) was added LiOH H₂O (30 mg, 0.714 mmol). The reaction mixture was stirred for 12 h at 25° C. After completed, the solvent was concentrated under vacuum. The residue was diluted with water and neutralized with 2 N HCl and freed dried to afford the title compound (40 mg, crude). MS: M/e 274 (M+H)⁺

### Step B: (S)—N-(3-(3-((4-methyl-4H-1,2,4-triazol-3-yl)methyl)oxetan-3-yl)phenyl)-7-((3-methylpiperidin-1-yl)methyl)-1H-pyrrolo[3,2-b]pyridine-5-carboxamide

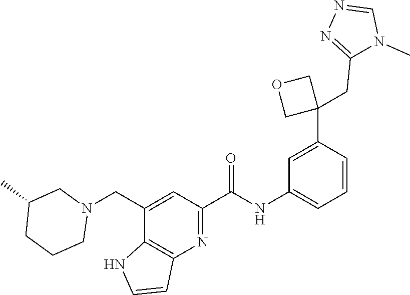

To a solution of (S)-7-((3-methylpiperidin-1-yl)methyl)-1H-pyrrolo[3,2-b]pyridine-5-carboxylic acid (20 mg, crude), 3-(3-((4-methyl-4H-1,2,4-triazol-3-yl)methyl)oxetan-3-yl)aniline (17 mg, 0.069 mmol), DIEA (20 mg, 0.155 mmol) in DMF (2 mL) was added HATU (40 mg, 0.15 mmol). The reaction mixture was stirred for 1 h at 25° C. After completed, the reaction was quenched with aq NH₄Cl (10 mL) and extracted with DCM (3×20 mL), dried over Na₂SO₄ and concentrated under vacuum. The residue was purified by Prep-HPLC to afford the title compound (15 mg, 43%). ¹H NMR (400 MHz, CD₃OD) δ 8.19 (s, 1H), 7.96 (s, 1H), 7.73 (d, J=8.2 Hz, 1H), 7.69 (d, J=3.2 Hz, 1H), 7.37 (s, 1H), 7.31 (t, J=8.0 Hz, 1H), 6.73 (d, J=3.2 Hz, 1H), 6.57 (d, J=7.8 Hz, 1H), 5.09-5.03 (m, 4H), 3.87 (s, 2H), 3.64 (s, 2H), 2.90-2.83 (m, 5H), 2.06-1.95 (m, 1H), 1.77-1.64 (m, 5H), 0.98-0.88 (m, 1H), 0.85 (d, J=5.8 Hz, 3H) ppm. MS: M/e 500 (M+H)⁺.

### Compound B1: N-(3-((1r,3S)-3-cyano-1-((4-methyl-4H-1,2,4-triazol-3-yl)methyl)cyclobutyl)phenyl)-7-(((S)-3-methylpiperidin-1-yl)methyl)-1H-pyrrolo[3,2-b]pyridine-5-carboxamide

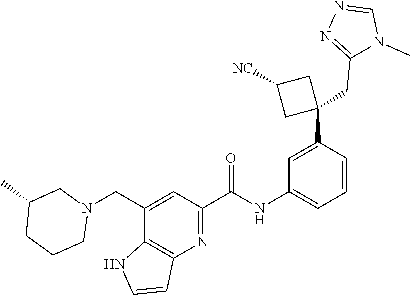

The compound B7 was synthesized starting from the corresponding starting materials according the similar procedures described as those of Compound B6 to afford the title compound (15 mg, 42%). ¹H NMR (400 MHz, CD₃OD) δ 8.17 (s, 1H), 7.97 (s, 1H), 7.73 (d, J=8.0 Hz, 1H), 7.70 (d, J=3.2 Hz, 1H), 7.54 (s, 1H), 7.31 (t, J=8.0 Hz, 1H), 6.77-6.72 (m, 2H), 3.89 (s, 2H), 3.41 (s, 2H), 3.24-3.12 (m, 1H), 3.05-2.93 (m, 4H), 2.92-2.82 (m, 2H), 2.81 (s, 3H), 2.08-1.96 (m, 1H), 1.79-1.63 (m, 5H), 0.99-0.88 (m, 1H), 0.85 (d, J=5.6 Hz, 3H) ppm. MS: M/e 523 (M+H)⁺.

### Compound B2: N-(3-((1s,3R)-3-(cyanomethyl)-1-(4-methyl-4H-1,2,4-triazol-3-yl)cyclobutyl)phenyl)-7-(((S)-3-methylpiperidin-1-yl)methyl)-1H-pyrazolo[4,3-b]pyridine-5-carboxamide

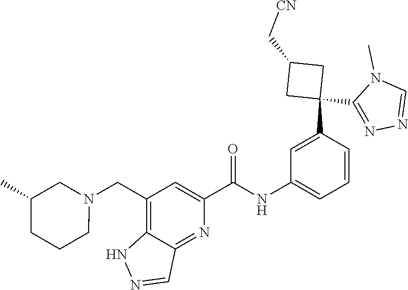 [SMILES: C[C@H]1CCCN(Cc2cc(C(=O)Nc3cccc([C@]4(c5nncn5C)C[C@@H](CC#N)C4)c3)nc3cn[nH]c23)C1]

### Step 1: 4-bromo-6-chloro-2-methylpyridin-3-amine

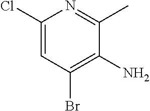

To a solution of 6-chloro-2-methylpyridin-3-amine (15 g, 105.20 mmol) in CH3CN (150 mL) was added NBS (22.47 g, 126.24 mmol) at 0° C. The resulting mixture was stirred at 20° C. for 16 h, then quenched with H₂O and extracted with EtOAc (300 mL, 100 mL). The combined organic layers were washed with brine (200 mL), and dried over Na₂SO₄. After filtration and concentration, the resulting residue was purified by column chromatography (SiO₂, PE:EA=100/1, 1/1) to give the product (6 g, 25.75% yield) as off-white solid. MS(ESI) m/e [M+1]221.4; 1H NMR (400 MHz, CDCl3) δ=7.28 (s, 1H), 4.24-3.82 (m, 2H), 2.44 (s, 3H) ppm.

### Step 2: 7-bromo-5-chloro-1H-pyrazolo[4,3-b]pyridine

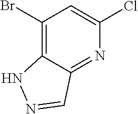 [SMILES: Clc1cc(Br)c2[nH]ncc2n1]

Isopentyl nitrite (4.76 g, 40.64 mmol) was dropwise added to an ice cold suspension of 4-bromo-6-chloro-2-methylpyridin-3-amine (6 g, 27.09 mmol), KOAc (5.32 g, 54.18 mmol) and AcOH (52.06 g, 866.89 mmol) in toluene (125 mL) under N₂ atmosphere. A reflux condenser was inserted and the reaction mixture was heated at 30° C. over 4 h. After which most of the solvent was removed under vacuo. The obtained residue was dissolved in ethyl acetate and carefully washed with saturated aqueous NaHCO₃ solution ensuring that pH 8-9 was obtained. The organic layer was washed with brine, dried (Na₂SO₄) and concentrated to a crude material which was purified by column chromatography (SiO₂, Petroleum ether/Ethyl acetate=100/1 to 0/1) to give the product (1.8 g, 28.58% yield) as off-white solid. MS(ESI) m/e [M+1]232; 1H NMR (400 MHz, CDCl3-d) 6=12.99 (br s, 1H), 8.21 (s, 1H), 7.47 (s, 1H) ppm.

### Step 3: 5-chloro-7-vinyl-1H-pyrazolo[4,3-b]pyridine

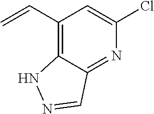

A mixture of 7-bromo-5-chloro-1H-pyrazolo[4,3-b]pyridine (1.3 g, 5.59 mmol), potassium trifluoro(vinyl)borate (823.99 mg, 6.15 mmol), Pd(dppf)Cl₂ (409.19 mg, 559.22 mol) and K₂CO₃ (2.32 g, 16.78 mmol) in Dioxane (90 mL) and H₂O (30 mL) was degassed and purged with N₂ for 3 times, and then the mixture was stirred at 100° C. for 5 h under N₂ atmosphere. The reaction mixture was added addition H₂O 20 mL at 20° C., and then extracted with EtOAc (120 mL, 40 mL). The combined organic layers were washed with brine (60 mL, 30 mL), dried over Na₂SO₄, filtered and concentrated under reduced pressure to give a residue. The residue was purified by column chromatography (SiO₂, Petroleum ether/Ethyl acetate=100/1 to 0/1) to give product (330 mg, yield 32.86%). MS(ESI) m/e [M+1]180; 1H NMR (400 MHz, DMSO-d6) δ=13.84 (br s, 1H), 8.31 (br s, 1H), 7.61 (br s, 1H), 7.10 (br dd, J=11.2, 17.6 Hz, 1H), 6.46 (br d, J=17.6 Hz, 1H), 5.86 (d, J=11.2 Hz, 1H) ppm.

### Step 4: 5-chloro-1H-pyrazolo[4,3-b]pyridine-7-carbaldehyde

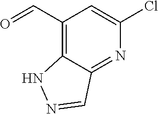 [SMILES: O=Cc1cc(Cl)nc2cn[nH]c12]

To a mixture of 5-chloro-7-vinyl-1H-pyrazolo[4,3-b]pyridine (400 mg, 2.23 mmol) and K₂OsO₄·H₂O (82.06 mg, 222.71 mol) in Dioxane (10 mL) and H₂O (2 mL) was added NaIO₄ (1.91 g, 8.91 mmol) in one portion at 20° C. under N₂. The mixture was stirred at 20° C. and stirred for 16 hours. The reaction mixture was quenched by addition H₂O 10 mL at 20° C., and then extracted with DCM (30 mL, 10 mL). The combined organic layers were washed with brine (30 mL, 10 mL), dried over Na₂SO₄, filtered and concentrated under reduced pressure to give the crude product (400 mg, crude), which was used to the next step without purification. MS(ESI) m/e [M+1] 182.5

### Step 5: (S)-5-chloro-7-((3-methylpiperidin-1-yl)methyl)-1H-pyrazolo[4,3-b]pyridine

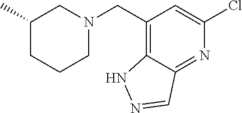 [SMILES: C[C@H]1CCCN(Cc2cc(Cl)nc3cn[nH]c23)C1]

To a mixture of 5-chloro-1H-pyrazolo[4,3-b]pyridine-7-carbaldehyde (430 mg, 2.37 mmol), (S)-3-methylpiperidine hydrochloride (642.40 mg, 4.74 mmol) and TEA (479.26 mg, 4.74 mmol) in DCM (5 mL) was added NaBH(OAc)₃ (1 g, 4.74 mmol) in one portion at 20° C. The mixture was stirred at 20° C. for 12 h. The mixture was poured into water (2 mL) and extracted with DCM (9 mL, 3 mL). The combined organic phase was washed with brine (5 mL), dried over Na₂SO₄, filtered and concentrated to give the residue, which was purified by column chromatography (SiO₂, Petroleum ether/Ethyl acetate=100/1 to 0/1) to give the product (300 mg, 47.85% yield) as a yellow solid. MS(ESI) m/e [M+1]265; 1H NMR (400 MHz, DMSO-d6) δ=13.48 (br s, 1H), 8.25 (s, 1H), 7.32 (s, 1H), 3.79 (s, 2H), 2.81-2.69 (m, 2H), 2.02-1.90 (m, 1H), 1.75-1.46 (m, 5H), 0.90-0.77 (m, 4H) ppm.

### Step 6: (S)-5-chloro-7-((3-methylpiperidin-1-yl)methyl)-1H-pyrazolo[4,3-b]pyridine

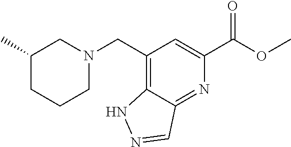

A mixture of (2-bromo-7,7-dimethyl-6,7-dihydro-5H-cyclopenta[b]pyridin-4-yl)methanol (100 mg, 377.71 μmol), Pd(dppf)Cl2 (55.27 mg, 75.54 mol), and TEA (76.44 mg, 755.42 mol) in MeOH (5 mL) was degassed and purged with CO for 3 times, The mixture was stirred under CO (50 psi) at 80° C. for 5 hours. The reaction mixture was filtered and the filter was concentrated. The crude product was purified by column chromatography (SiO₂, Petroleum ether/Ethyl acetate=100/1 to 0/1) to give product (90 mg, 82.64% yield). MS(ESI) m/e [M+1]289; 1H NMR (400 MHz, DMSO-d6) δ=13.57 (br s, 1H), 8.46 (s, 1H), 7.99 (s, 1H), 3.90 (s, 3H), 3.84 (s, 2H), 2.82-2.70 (m, 2H), 1.98-1.91 (m, 1H), 1.75-1.47 (m, 5H), 0.94-0.74 (m, 4H) ppm.

### Step 7: lithium (S)-7-((3-methylpiperidin-1-yl)methyl)-1H-pyrazolo[4,3-b]pyridine-5-carboxylate

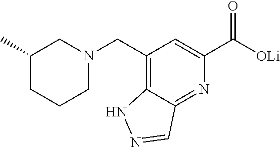

To a solution of methyl (S)-7,7-dimethyl-4-((3-methylpiperidin-1-yl)methyl)-6,7-dihydro-5H-cyclopenta[b]pyridine-2-carboxylate (100 mg, 346.81 mol) in MeOH/THF/H₂O (0.5 mL/0.5 mL/0.5 mL) was added LiOH·H₂O (21.83 mg, 520.21 mol). The mixture was stirred for 12 hr at rt. The mixture was filtered and concentrated to give the crude product (70 mg, crude), which was used to the next step without purification. MS(ESI) m/e [M+1]275; 1H NMR (400 MHz, DMSO-d6) δ=7.98 (s, 1H), 7.57 (s, 1H), 3.85 (s, 2H), 2.93-2.76 (m, 2H), 1.95-1.85 (m, 1H), 1.72-1.52 (m, 8H), 0.90-0.75 (m, 4H) ppm.

### Step 8: N-(3-((1s,3R)-3-(cyanomethyl)-1-(4-methyl-4H-1,2,4-triazol-3-yl)cyclobutyl)phenyl)-7-(((S)-3-methylpiperidin-1-yl)methyl)-1H-pyrazolo[4,3-b]pyridine-5-carboxamide

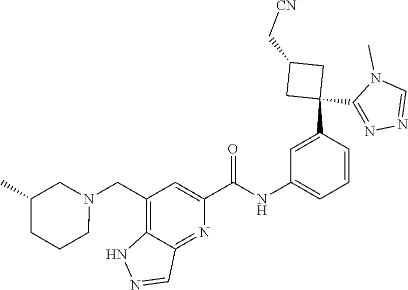 [SMILES: C[C@H]1CCCN(Cc2cc(C(=O)Nc3cccc([C@]4(c5nncn5C)C[C@@H](CC#N)C4)c3)nc3cn[nH]c23)C1]

To a solution of lithium (S)-7-((3-methylpiperidin-1-yl)methyl)-1H-pyrazolo[4,3-b]pyridine-5-carboxylate (10 mg, 20.57 mol) in DMF (1 mL) was added DIEA (4.83 mg, 37.41 mol) and HATU (9.25 mg, 24.31 μmol). The mixture was stirred for 5 mins at rt, then 2-((1s, 3s)-3-(3-aminophenyl)-3-(4-methyl-4H-1,2,4-triazol-3-yl)cyclobutyl)acetonitrile (5 mg, 18.70 μmol) was added. The mixture was stirred for 12 hrs at rt. The mixture was poured into water (1 mL) and extracted with DCM (2 mL, 1 mL). The combined organic phase was filtered and concentrated to give the residue, which was purified by prep-HPLC (column=Waters Xbridge Prep OBD C18 (150*40 mm*10 um); mobile phase=water (NH4HCO3)-ACN, B %=25%-55%; 8 min) to give the product (2.75 mg, 28.08% yield). MS(ESI) m/e [M+1]524; 1H NMR (400 MHz, DMSO-d6) δ=13.75-13.41 (m, 1H), 10.74 (s, 1H), 8.49 (s, 1H), 8.32 (s, 1H), 8.11 (s, 1H), 8.01-7.89 (m, 2H), 7.36 (t, J=8.0 Hz, 1H), 7.00 (d, J=8.0 Hz, 1H), 3.88 (s, 2H), 3.21 (s, 3H), 2.93-2.68 (m, 9H), 1.99 (br d, J=3.6 Hz, 1H), 1.76-1.50 (m, 5H), 0.81 (d, J=5.6 Hz, 4H) ppm.

### Compound B3: N-(3-(3-((4-methyl-4H-1,2,4-triazol-3-yl)methyl)oxetan-3-yl)phenyl)-7-((3-(trifluoromethyl)piperidin-1-yl)methyl)-1H-pyrrolo[3,2-b]pyridine-5-carboxamide

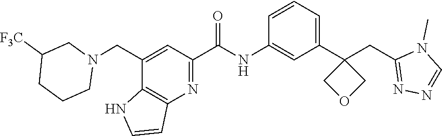 [SMILES: Cn1cnnc1CC1(c2cccc(NC(=O)c3cc(CN4CCCC(C(F)(F)F)C4)c4[nH]ccc4n3)c2)COC1]

The compound B9 was synthesized starting from the corresponding starting materials according the similar procedures described as those of Compound B5 to afford the title compound (15 mg, 38%). ¹H NMR (400 MHz, CD₃OD) δ 8.19 (s, 1H), 7.95 (s, 1H), 7.74 (d, J=8.2 Hz, 1H), 7.69 (d, J=3.2 Hz, 1H), 7.37 (s, 1H), 7.31 (t, J=8.0 Hz, 1H), 6.74 (d, J=3.2 Hz, 1H), 6.59 (d, J=7.8 Hz, 1H), 5.10-5.02 (m, 4H), 4.03-3.86 (m, 2H), 3.64 (s, 2H), 3.10 (d, J=10.6 Hz, 1H), 2.89-2.81 (m, 4H), 2.63-2.51 (m, 1H), 2.16-1.93 (m, 3H), 1.80-1.63 (m, 2H), 1.41-1.27 (m, 1H). MS: M/e 554 (M+H)⁺.

### Compound B4: N-(3-((1s,3R)-3-(cyanomethyl)-1-(4-methyl-4H-1,2,4-triazol-3-yl)cyclobutyl)phenyl)-3-methyl-7-(((S)-3-methylpiperidin-1-yl)methyl)-1H-pyrrolo[3,2-b]pyridine-5-carboxamide

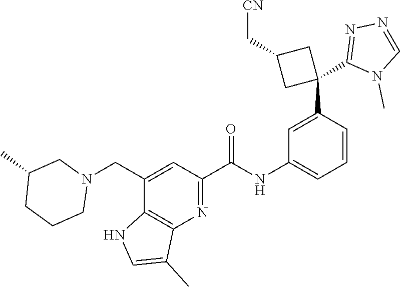 [SMILES: Cc1c[nH]c2c(CN3CCC[C@H](C)C3)cc(C(=O)Nc3cccc([C@]4(c5nncn5C)C[C@@H](CC#N)C4)c3)nc12]

### Step A: methyl (S)-3-iodo-7-((3-methylpiperidin-1-yl)methyl)-1H-pyrrolo[3,2-b]pyridine-5-carboxylate

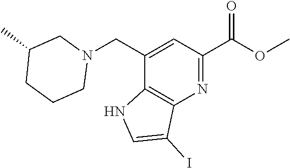

To a solution of methyl (S)-7-((3-methylpiperidin-1-yl)methyl)-1H-pyrrolo[3,2-b]pyridine-5-carboxylate (40 mg, 0.139 mmol) in ACN (5 mL) was added NIS (35 mg, 0.156 mmol). The reaction mixture was stirred for 0.5 h at 25° C. After completed, the solvent was removed by in vacuo. The residue was purified by Prep-TLC (DCM/MeOH=15/1) to afford the title compound (70 mg, crude). MS: M/e 413.9 (M+H)⁺.

### Step B: methyl (S)-3-iodo-7-((3-methylpiperidin-1-yl)methyl)-1-((2-(trimethylsilyl)ethoxy)methyl)-1H-pyrrolo[3,2-b]pyridine-5-carboxylate

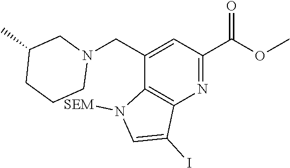

To a solution of methyl (S)-3-iodo-7-((3-methylpiperidin-1-yl)methyl)-1H-pyrrolo[3,2-b]pyridine-5-carboxylate (70 mg, crude) in THF (5 mL) was added NaH (9 mg, 0.225 mmol). After stirring for 10 min, SEMCl (43 mg, 0.257) was added dropwise. The reaction mixture was stirred for 12 h at 25° C. After completed, the reaction was quenched with aq NH₄Cl (20 mL) and extracted with DCM/MeOH (10/1, 3×50 mL), dried over Na₂SO₄ and concentrated under vacuum. The residue was purified by Prep-TLC (DCM/MeOH=15/1) to afford the title compound (45 mg, 60%). MS: M/e 543.9 (M+H)⁺.

### Step C: methyl (S)-3-methyl-7-((3-methylpiperidin-1-yl)methyl)-1-((2-(trimethylsilyl)ethoxy)methyl)-1H-pyrrolo[3,2-b]pyridine-5-carboxylate

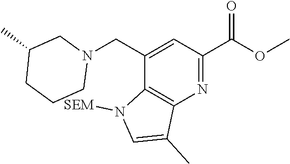

To a solution of methyl (S)-3-iodo-7-((3-methylpiperidin-1-yl)methyl)-1-((2-(trimethylsilyl)ethoxy)methyl)-1H-pyrrolo[3,2-b]pyridine-5-carboxylate (35 mg, 0.064 mmol), tetramethyltin (23 mg, 0.128 mmol) in DMF (5 mL) was added LiCl (6 mg, 0.143 mmol) and PdCl₂(PPh₃)₂(5 mg, 0.007 mmol) under N₂. The reaction mixture was stirred for 12 h at 110° C. After completed, the reaction was quenched with aq NH₄Cl (20 mL) and extracted with EtOAc (3×50 mL), dried over Na₂SO₄ and concentrated under vacuum. The residue was purified by Prep-TLC (PE/EtOAc=4/1) to afford the title compound (14 mg, impure) and (S)-3-methyl-7-((3-methylpiperidin-1-yl)methyl)-1-((2-(trimethylsilyl)ethoxy)methyl)-1H-pyrrolo[3,2-b]pyridine-5-carboxylic acid (35 mg, crude). ¹H NMR (400 MHz, CD₃OD) δ 8.02 (s, 1H), 7.65 (s, 1H), 5.86 (s, 2H), 4.16 (s, 2H), 4.07 (s, 3H), 3.70-3.65 (m, 1H), 3.59 (t, J=8.0 Hz, 2H), 3.10-2.91 (m, 2H), 2.48 (s, 3H), 2.40-2.24 (m, 1H), 2.14-1.97 (m, 1H), 1.85-1.60 (m, 5H), 1.14-1.03 (m, 1H), 1.00-0.88 (m, 3H), −0.00 (s, 9H) ppm. MS: M/e 432 (M+H)⁺.

### Step D: (S)-3-methyl-7-((3-methylpiperidin-1-yl)methyl)-1-((2-(trimethylsilyl)ethoxy)methyl)-1H-pyrrolo[3,2-b]pyridine-5-carboxylic acid

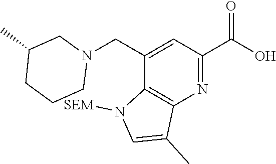 [SMILES: Cc1cn(COCC[Si](C)(C)C)c2c(CN3CCC[C@H](C)C3)cc(C(=O)O)nc12]

To a solution of methyl (S)-3-methyl-7-((3-methylpiperidin-1-yl)methyl)-1-((2-(trimethylsilyl)ethoxy)methyl)-1H-pyrrolo[3,2-b]pyridine-5-carboxylate (14 mg, impure) in MeOH (5 mL)/H₂O (0.5 mL) was added LiOH H₂O (8 mg, 0.190 mmol). The reaction mixture was stirred for 12 h at 25° C. After completed, the solvent was concentrated under vacuum. The residue was diluted with water and neutralized with 2 N HCl and freed dried to afford the title compound (15 mg, crude). MS: M/e 418 (M+H)⁺.

### Step E: N-(3-((1s,3R)-3-(cyanomethyl)-1-(4-methyl-4H-1,2,4-triazol-3-yl)cyclobutyl)phenyl)-3-methyl-7-(((S)-3-methylpiperidin-1-yl)methyl)-1-((2-(trimethylsilyl)ethoxy)methyl)-1H-pyrrolo[3,2-b]pyridine-5-carboxamide

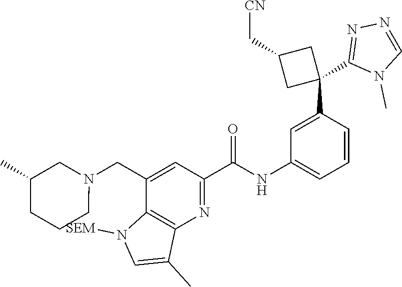 [SMILES: Cc1cn(COCC[Si](C)(C)C)c2c(CN3CCC[C@H](C)C3)cc(C(=O)Nc3cccc([C@]4(c5nncn5C)C[C@@H](CC#N)C4)c3)nc12]

To a solution of (S)-3-methyl-7-((3-methylpiperidin-1-yl)methyl)-1-((2-(trimethylsilyl)ethoxy)methyl)-1H-pyrrolo[3,2-b]pyridine-5-carboxylic acid (90 mg, 0.215 mmol), 2-((1s, 3s)-3-(3-aminophenyl)-3-(4-methyl-4H-1,2,4-triazol-3-yl)cyclobutyl)acetonitrile (60 mg, 0.224 mmol), DIEA (56 mg, 0.434 mmol) in DMF (2 mL) was added HATU (125 mg, 0.328 mmol). The reaction mixture was stirred for 1 h at 25° C. After completed, the reaction was quenched with aq NH₄Cl (20 mL) and extracted with DCM (3×30 mL), dried over Na2SO4 and concentrated under vacuum. The residue was purified by Prep-TLC (DCM/MeOH=15/1) to afford the title compound (140 mg, 97%). MS: M/e 667 (M+H)⁺.

### Step F: N-(3-((1s,3R)-3-(cyanomethyl)-1-(4-methyl-4H-1,2,4-triazol-3-yl)cyclobutyl)phenyl)-3-methyl-7-(((S)-3-methylpiperidin-1-yl)methyl)-1H-pyrrolo[3,2-b]pyridine-5-carboxamide

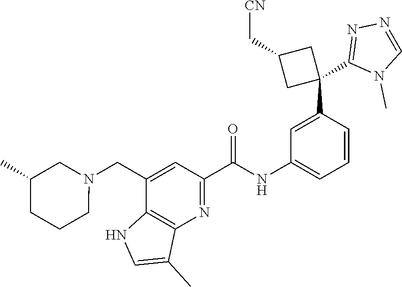 [SMILES: Cc1c[nH]c2c(CN3CCC[C@H](C)C3)cc(C(=O)Nc3cccc([C@]4(c5nncn5C)C[C@@H](CC#N)C4)c3)nc12]

To a solution of N-(3-((1s,3R)-3-(cyanomethyl)-1-(4-methyl-4H-1,2,4-triazol-3-yl)cyclobutyl)phenyl)-3-methyl-7-(((S)-3-methylpiperidin-1-yl)methyl)-1-((2-(trimethylsilyl)ethoxy)methyl)-1H-pyrrolo[3,2-b]pyridine-5-carboxamide (140 mg, 0.209 mmol) in DCM (2 mL) was added TFA (8 mL). The reaction mixture was stirred for 36 h at 25° C. After completed, the solvent was removed by in vacuo. The residue was dissolved in MeOH (10 mL) and Ethylenediamine (0.5 mL) was added. The reaction mixture was stirred for 12 h at 25° C. After completed, the solvent was removed by in vacuo. The residue was purified by prep-HPLC to afford the title compound (60 mg, 53%). 1H NMR (400 MHz, CD3OD) δ 8.32 (s, 1H), 7.96 (d, J=4.6 Hz, 2H), 7.77 (d, J=9.0 Hz, 1H), 7.47-7.39 (m, 2H), 7.11 (d, J=7.8 Hz, 1H), 3.84 (s, 2H), 3.33 (s, 3H), 3.08-2.98 (m, 2H), 2.96-2.78 (m, 5H), 2.65 (d, J=6.0 Hz, 2H), 2.44 (s, 3H), 2.05-1.92 (m, 1H), 1.76-1.60 (m, 5H), 0.96-0.87 (m, 1H), 0.83 (d, J=5.8 Hz, 3H). MS: M/e 537 (M+H)⁺.

### Compound B11: N-(3-((1s,3R)-3-(cyanomethyl)-1-(4-methyl-4H-1,2,4-triazol-3-yl)cyclobutyl)phenyl)-3-fluoro-7-(((S)-3-methylpiperidin-1-yl)methyl)-1H-pyrrolo[3,2-b]pyridine-5-carboxamide

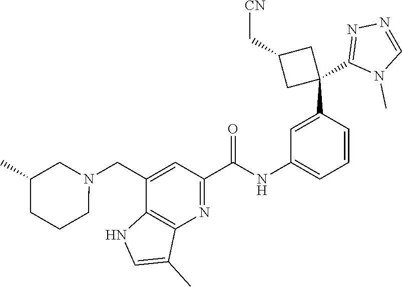 [SMILES: Cc1c[nH]c2c(CN3CCC[C@H](C)C3)cc(C(=O)Nc3cccc([C@]4(c5nncn5C)C[C@@H](CC#N)C4)c3)nc12]

### Step A: methyl 7-chloro-3-fluoro-1H-pyrrolo[3,2-b]pyridine-5-carboxylate

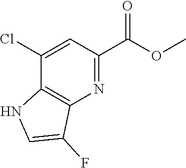

To a solution of methyl 7-chloro-3-fluoro-1H-pyrrolo[3,2-b]pyridine-5-carboxylate (1.5 g, 7.1 mmol) and NaHCO₃ (1.2 g, 14.28 mmol) in DMF (15 mL) was added selectfluor (3.6 g, 10.17 mmol). The reaction mixture was stirred for 12 h at 60° C. After completed, the reaction was quenched with aq NH₄Cl (30 mL) and extracted with EtOAc (3×80 mL), dried over Na2SO4 and concentrated under vacuum. The residue was purified by column chromatography on silica gel eluting with ethyl acetate in petroleum ether (70%) to afford the title compound (850 mg, impure). MS: M/e 229, 231 (M+H)+.

### Step B: methyl 7-chloro-3-fluoro-1-((2-(trimethylsilyl)ethoxy)methyl)-1H-pyrrolo[3,2-b]pyridine-5-carboxylate

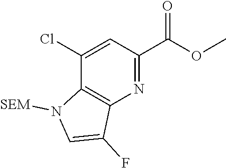 [SMILES: COC(=O)c1cc(Cl)c2c(n1)c(F)cn2COCC[Si](C)(C)C]

To a solution of methyl 7-chloro-3-fluoro-1H-pyrrolo[3,2-b]pyridine-5-carboxylate (1 g, impure) in THF (20 mL) was added NaH (210 mg, 5.25 mmol) at 0° C. After stirring for 10 min at 0° C., SEMCl (1.1 g, 6.58 mmol) was added dropwise. The reaction mixture was stirred for 12 h at 25° C. After completed, the reaction was quenched with aq NH₄Cl (20 mL) and extracted with DCM (3×50 mL), dried over Na2SO4 and concentrated under vacuum. The residue was purified by column chromatography on silica gel eluting with ethyl acetate in petroleum ether (25%) to afford the title compound (980 mg, impure). MS: M/e 359, 361 (M+H)⁺.

### Step C: methyl 3-fluoro-1-((2-(trimethylsilyl)ethoxy)methyl)-7-vinyl-1H-pyrrolo[3,2-b]pyridine-5-carboxylate

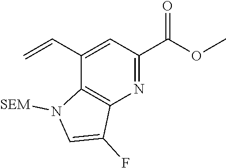 [SMILES: C=Cc1cc(C(=O)OC)nc2c(F)cn(COCC[Si](C)(C)C)c12]

To a solution of methyl 7-chloro-3-fluoro-1-((2-(trimethylsilyl)ethoxy)methyl)-1H-pyrrolo[3,2-b]pyridine-5-carboxylate (980 mg, impure), potassium trifluoro(vinyl)borate (550 mg, 4.1 mmol) in Dioxane (20 mL)/H2O (2 mL) was added Xphos G2 Pd (110 mg, 0.139 mmol) and Cs2CO3 (1.8 g, 5.5 mmol) under N2. The reaction mixture was stirred for 2 h at 80° C. After completed, the mixture was filtered and the filate was collected and concentrated under vacuum. The residue was purified by column chromatography on silica gel eluting with ethyl acetate in petroleum ether (20%) to afford the title compound (580 mg, impure). MS: M/e 351 (M+H)+.

### Step D: methyl 3-fluoro-7-formyl-1-((2-(trimethylsilyl)ethoxy)methyl)-1H-pyrrolo[3,2-b]pyridine-5-carboxylate

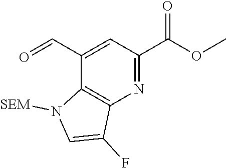 [SMILES: COC(=O)c1cc(C=O)c2c(n1)c(F)cn2COCC[Si](C)(C)C]

To a solution of methyl 3-fluoro-1-((2-(trimethylsilyl)ethoxy)methyl)-7-vinyl-1H-pyrrolo[3,2-b]pyridine-5-carboxylate (580 mg, impure), NaIO4 (1.4 g, 6.54 mmol) in Dioxane (15 mL)/H2O (3 mL) was added K2OsO4 2H2O (60 mg, 0.163 mmol). The reaction mixture was stirred for 2 h at 25° C. After completed, the reaction was quenched with water (20 mL) and extracted with EtOAc (3×50 mL), dried over Na2SO4 and concentrated under vacuum. The residue was purified by column chromatography on silica gel eluting with ethyl acetate in petroleum ether (25%) to afford the title compound (340 mg, impure). MS: M/e 353 (M+H)⁺.

### Step E: methyl (S)-3-fluoro-7-((3-methylpiperidin-1-yl)methyl)-1-((2-(trimethylsilyl) ethoxy)methyl)-1H-pyrrolo[3,2-b]pyridine-5-carboxylate

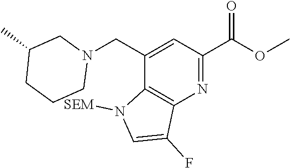

To a solution of methyl 3-fluoro-7-formyl-1-((2-(trimethylsilyl)ethoxy)methyl)-1H-pyrrolo[3,2-b]pyridine-5-carboxylate (340 mg, impure), (S)-3-methylpiperidine hydrochloride (190 mg, 1.39 mmol), Et3N (150 mg, 1.48 mmol) in DCM (10 mL) was added NaBH(OAc)3 (400 mg, 1.88 mmol). The reaction mixture was stirred for 12 h at 25° C. After completed, the reaction was quenched with aq NaHCO3 (20 mL) and extracted with DCM (3×50 mL), dried over Na2SO4 and concentrated under vacuum. The residue was purified by column chromatography on silica gel eluting with ethyl acetate in petroleum ether (25%) to afford the title compound (290 mg, 69%). MS: M/e 436 (M+H)+.

### Step F: (S)-3-fluoro-7-((3-methylpiperidin-1-yl)methyl)-1-((2-(trimethylsilyl)ethoxy) methyl)-1H-pyrrolo[3,2-b]pyridine-5-carboxylic acid

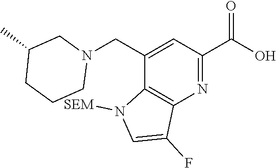 [SMILES: C[C@H]1CCCN(Cc2cc(C(=O)O)nc3c(F)cn(COCC[Si](C)(C)C)c23)C1]

To a solution of methyl (S)-3-fluoro-7-((3-methylpiperidin-1-yl)methyl)-1-((2-(trimethylsilyl) ethoxy)methyl)-1H-pyrrolo[3,2-b]pyridine-5-carboxylate (60 mg, 0.138 mmol) in EtOH (5 mL)/H2O (0.5 mL) was added LiOH H2O (30 mg, 0.714 mmol). The reaction mixture was stirred for 12 h at 25° C. After completed, the solvent was concentrated under vacuum. The residue was diluted with water and neutralized with 2 N HCl and freed dried to afford the title compound (60 mg, crude). MS: M/e 422 (M+H)+.

### Step G: N-(3-((1s,3R)-3-(cyanomethyl)-1-(4-methyl-4H-1,2,4-triazol-3-yl)cyclobutyl) phenyl)-3-fluoro-7-(((S)-3-methylpiperidin-1-yl)methyl)-1-((2-(trimethylsilyl)ethoxy) methyl)-1H-pyrrolo[3,2-b]pyridine-5-carboxamide

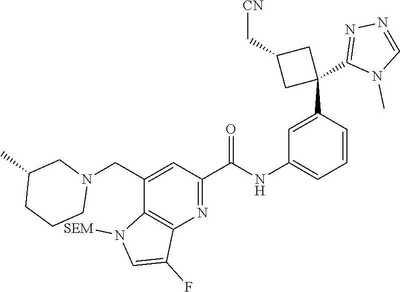 [SMILES: C[C@H]1CCCN(Cc2cc(C(=O)Nc3cccc([C@]4(c5nncn5C)C[C@@H](CC#N)C4)c3)nc3c(F)cn(COCC[Si](C)(C)C)c23)C1]

To a solution of (S)-3-fluoro-7-((3-methylpiperidin-1-yl)methyl)-1-((2-(trimethylsilyl)ethoxy) methyl)-1H-pyrrolo[3,2-b]pyridine-5-carboxylic acid (100 mg, crude), 2-((1s, 3s)-3-(3-aminophenyl)-3-(4-methyl-4H-1,2,4-triazol-3-yl)cyclobutyl)acetonitrile (70 mg, 0.262 mmol), DIEA (65 mg, 0.503 mmol) in DMF (3 mL) was added HATU (140 mg, 0.368 mmol). The reaction mixture was stirred for 1 h at 25° C. After completed, the reaction was quenched with aq NH4Cl (20 mL) and extracted with DCM (3×30 mL), dried over Na2SO4 and concentrated under vacuum. The residue was purified by Prep-TLC (DCM/MeOH=15/1) to afford the title compound (110 mg, 70%). MS: M/e 671 (M+H)+.

### Step H: N-(3-((1s,3R)-3-(cyanomethyl)-1-(4-methyl-4H-1,2,4-triazol-3-yl)cyclobutyl) phenyl)-3-fluoro-7-(((S)-3-methylpiperidin-1-yl)methyl)-1H-pyrrolo[3,2-b]pyridine-5-carboxamide

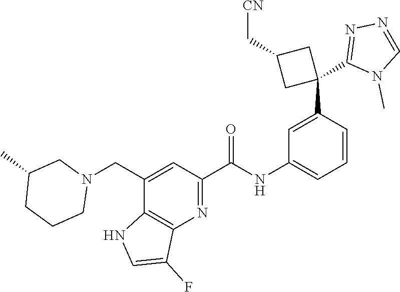 [SMILES: C[C@H]1CCCN(Cc2cc(C(=O)Nc3cccc([C@]4(c5nncn5C)C[C@@H](CC#N)C4)c3)nc3c(F)c[nH]c23)C1]

To a solution of N-(3-((1s,3R)-3-(cyanomethyl)-1-(4-methyl-4H-1,2,4-triazol-3-yl)cyclobutyl) phenyl)-3-fluoro-7-(((S)-3-methylpiperidin-1-yl)methyl)-1-((2-(trimethylsilyl)ethoxy) methyl)-1H-pyrrolo[3,2-b]pyridine-5-carboxamide (110 mg, 0.164 mmol) in DCM (2 mL) was added TFA (8 mL). The reaction mixture was stirred for 36 h at 25° C. After completed, the solvent was removed by in vacuo. The residue was dissolved in MeOH (10 mL) and Ethylenediamine (0.4 mL) was added. The reaction mixture was stirred for 12 h at 25° C. After completed, the solvent was removed by in vacuo. The residue was purified by prep-HPLC to afford the title compound (50 mg, 56%). 1H NMR (400 MHz, CD3OD) δ 8.33 (s, 1H), 8.03 (s, 1H), 7.97 (s, 1H), 7.82 (d, J=8.0 Hz, 1H), 7.55 (d, J=2.2 Hz, 1H), 7.42 (t, J=8.0 Hz, 1H), 7.11 (d, J=8.0 Hz, 1H), 3.87 (s, 2H), 3.33 (s, 3H), 3.08-3.00 (m, 2H), 2.99-2.89 (m, 1H), 2.88-2.77 (m, 4H), 2.66 (d, J=6.0 Hz, 2H), 2.05-1.96 (m, 1H), 1.79-1.62 (m, 5H), 0.99-0.88 (m, 1H), 0.85 (d, J=5.8 Hz, 3H). MS: M/e 541 (M+H)+.

### Compound B12: N-(3-{3-[(4-methyl-1,2,4-triazol-3-yl)methyl]oxetan-3-yl}phenyl)-4-{[(3S)-3-methylpiperidin-1-yl]methyl}pyrrolo[1,2-b]pyridazine-2-carboxamide

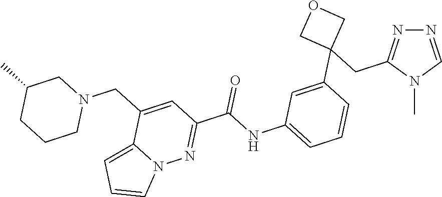 [SMILES: C[C@H]1CCCN(Cc2cc(C(=O)Nc3cccc(C4(Cc5nncn5C)COC4)c3)nn3cccc23)C1]

### Step 1: ethyl 5-(benzyloxy)-2,4-dioxopentanoate

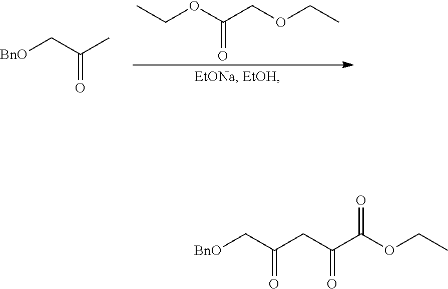

To a stirred mixture of 1-(benzyloxy)propan-2-one (5 g, 28.927 mmol, 1 equiv, 95%) and ethyl oxalate (5 g, 32.503 mmol, 1.12 equiv, 95%) in ethanol was added EtONa (13 g, 40.117 mmol, 1.39 equiv, 21%) dropwise at 0° C. The resulting mixture was stirred for 15 h at room temperature. The resulting mixture was concentrated under vacuum. The reaction was quenched with HCl (1 M) at 0° C. The resulting mixture was diluted with water. The resulting mixture was extracted with EtOAc (3×100 mL). The combined organic layers were washed with brine (1×80 mL), dried over anhydrous Na2SO4. After filtration, the filtrate was concentrated under reduced pressure. The residue was purified by silica gel column chromatography, eluted with CH2Cl2/PE (1:1) to afford ethyl 5-(benzyloxy)-2,4-dioxopentanoate (3.5 g, 33.56%) as a yellow oil. LC-MS (M+H)⁺:=265.

### Step 2: ethyl 4-[(benzyloxy)methyl]pyrrolo[1,2-b]pyridazine-2-carboxylate

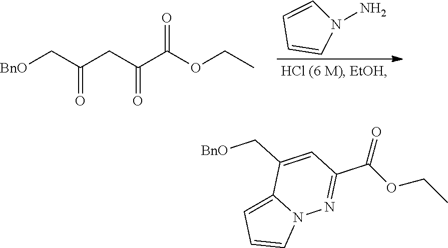

To a stirred mixture of ethyl 5-(benzyloxy)-2,4-dioxopentanoate (2.3 g, 6.379 mmol, 1.00 equiv, 73.3%) and pyrrol-1-amine (550 mg, 6.364 mmol, 1.00 equiv, 95%) in ethanol was added HCl (6M) (10 mL, 312.671 mmol, 49.13 equiv, 95%) dropwise at 0° C. The resulting mixture was stirred for 1 h at 0° C. The reaction was quenched with Water at 0° C. The resulting mixture was extracted with EtOAc (3×50 mL). The combined organic layers were washed with brine (1×30 mL), dried over anhydrous Na2SO4. After filtration, the filtrate was concentrated under reduced pressure. The residue was purified by silica gel column chromatography, eluted with PE/EA (9:1) to afford ethyl 4-[(benzyloxy)methyl]pyrrolo[1,2-b]pyridazine-2-carboxylate (1.4 g, 67.63%) as a yellow oil. LC-MS (M+H)⁺:=311.

### Step 3: ethyl 4-(hydroxymethyl)pyrrolo[1,2-b]pyridazine-2-carboxylate

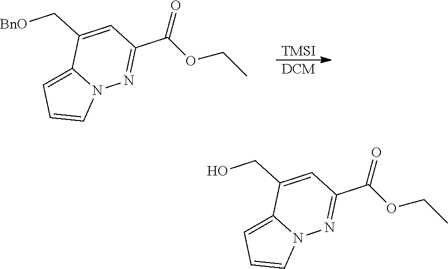

To a stirred solution of ethyl 4-[(benzyloxy)methyl]pyrrolo[1,2-b]pyridazine-2-carboxylate (1.4 g, 4.303 mmol, 1 equiv, 95.4%) in DCM was added TMSI (9.1 g, 43.205 mmol, 10.04 equiv, 95%) dropwise at 0° C. under nitrogen atmosphere. The resulting mixture was stirred for 14 h at room temperature under nitrogen atmosphere. The reaction was quenched with Water at 0° C. The resulting mixture was extracted with CH2Cl2 (3×50 mL). The combined organic layers were washed with brine (1×50 mL), dried over anhydrous Na2SO4. After filtration, the filtrate was concentrated under reduced pressure. The residue was purified by silica gel column chromatography, eluted with PE/EA (3:1) to afford ethyl 4-(hydroxymethyl)pyrrolo[1,2-b]pyridazine-2-carboxylate (630 mg, 64.94%) as a yellow solid. LC-MS (M+H)⁺:=221.

### Step 4: ethyl 4-formylpyrrolo[1,2-b]pyridazine-2-carboxylate

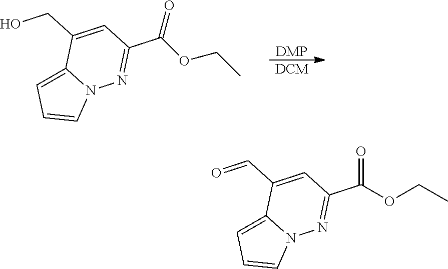

To a stirred solution of ethyl 4-(hydroxymethyl)pyrrolo[1,2-b]pyridazine-2-carboxylate (550 mg, 2.440 mmol, 1 equiv, 97.7%) in DCM was added Dess-Martin (1.7 g, 3.808 mmol, 1.56 equiv, 95%) in portions at 0° C. The resulting mixture was stirred for 4 h at room temperature. The resulting mixture was concentrated under vacuum. The residue was purified by silica gel column chromatography, eluted with PE/EA (9:1) to afford ethyl 4-formylpyrrolo[1,2-b]pyridazine-2-carboxylate (505 mg, 84.60%) as a red solid. LC-MS (M+H)⁺:=219.0.

### Step 5: ethyl 4-{[(3S)-3-methylpiperidin-1-yl]methyl}pyrrolo[1,2-b]pyridazine-2-carboxylate

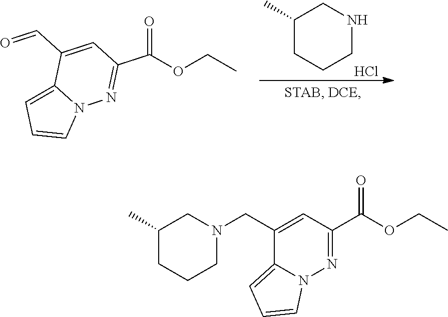

A mixture of ethyl 4-formylpyrrolo[1,2-b]pyridazine-2-carboxylate (480 mg, 1.962 mmol, 1 equiv, 89.2%) and (3S)-3-methylpiperidine hydrochloride (421 mg, 2.949 mmol, 1.50 equiv, 95%) in DCE was stirred for 2 h at 40° C. under nitrogen atmosphere. The mixture was allowed to cool down to room temperature. To the above mixture was added STAB (657 mg, 2.945 mmol, 1.50 equiv, 95%) in portions at room temperature. The resulting mixture was stirred for additional 3 h at 40° C. The resulting mixture was concentrated under vacuum. The residue was purified by silica gel column chromatography, eluted with PE/EA (6:4) to afford ethyl 4-{[(3S)-3-methylpiperidin-1-yl]methyl}pyrrolo[1,2-b]pyridazine-2-carboxylate (460 mg, 71.49%) as a yellow oil. LC-MS (M+H)⁺:=302.

### Step 6: 4-{[(3S)-3-methylpiperidin-1-yl]methyl}pyrrolo[1,2-b]pyridazine-2-carboxylic acid

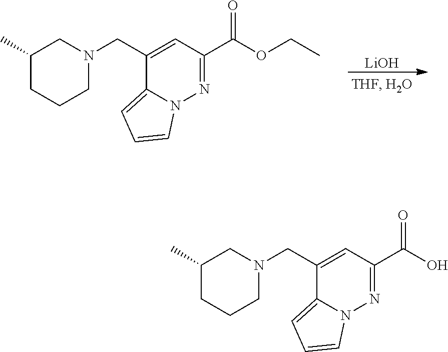

To a stirred solution of ethyl 4-{[(3S)-3-methylpiperidin-1-yl]methyl}pyrrolo[1,2-b]pyridazine-2-carboxylate (430 mg, 1.311 mmol, 1 equiv, 91.9%) in THF and H2O (2.5 mL, 137.386 mmol, 104.78 equiv, 99%) was added LiOH (66 mg, 2.618 mmol, 2.00 equiv, 95%). The resulting mixture was stirred for 12 h at room temperature. The residue was acidified to pH 6 with HCl (1 M). The resulting mixture was concentrated under vacuum. The residue was purified by reverse flash chromatography with the following conditions: column, C18 silica gel; mobile phase, MeCN in Water (0.1% FA), 10% to 30% gradient in 30 min; detector, UV 254 nm. This resulted in 4-{[(3S)-3-methylpiperidin-1-yl]methyl}pyrrolo[1,2-b]pyridazine-2-carboxylic acid (325 mg, 85.97%) as a yellow oil. LC-MS (M+H)⁺:=274.1.

### Step 7: N-(3-{3-[(4-methyl-1,2,4-triazol-3-yl)methyl]oxetan-3-yl}phenyl)-4-{[(3S)-3-methylpiperidin-1-yl]methyl}pyrrolo[1,2-b]pyridazine-2-carboxamide

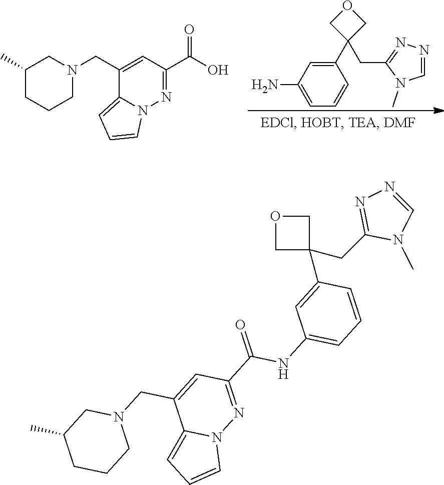

To a stirred mixture of 4-{[(3S)-3-methylpiperidin-1-yl]methyl}pyrrolo[1,2-b]pyridazine-2-carboxylic acid (32 mg, 0.111 mmol, 1.09 equiv, 94.8%) and HOBT (22 mg, 0.155 mmol, 1.51 equiv, 95%), EDCI (31 mg, 0.154 mmol, 1.50 equiv, 95%), Et3N (44 mg, 0.413 mmol, 4.04 equiv, 95%) in DMF stirred for 5 mins was added 3-{3-[(4-methyl-1,2,4-triazol-3-yl)methyl]oxetan-3-yl}aniline (25 mg, 0.102 mmol, 1.00 equiv, 99.9%) dropwise at room temperature. The resulting mixture was stirred for 12 h at 40° C. The residue was purified by reverse flash chromatography with the following conditions: column, C18 silica gel; mobile phase, MeCN in Water (0.1% NH3·H2O), 0% to 95% gradient in 15 min; detector, UV 254 nm. The crude product (55 mg) was purified by Prep-HPLC with the following conditions (2 #SHIMADZU (HPLC-01)): Column, XBridge Prep OBD C18 Column, 30*150 mm, 5 μm; mobile phase, Water (10 mmol/L NH4HCO3+0.1% NH3·H2O) and ACN (42% ACN up to 72% in 8 min); This resulted in N-(3-{3-[(4-methyl-1,2,4-triazol-3-yl)methyl]oxetan-3-yl}phenyl)-4-{[(3S)-3-methylpiperidin-1-yl]methyl}pyrrolo[1,2-b]pyridazine-2-carboxamide (22.6 mg, 43.41%). LC-MS (M+H)⁺:=500; ¹H NMR (300 MHz, DMSO-d6) δ 10.49 (s, 1H), 8.21 (s, 1H), 8.02-7.97 (m, 1H), 7.85-7.76 (m, 1H), 7.53-7.47 (m, 1H), 7.32-7.23 (m, 2H), 7.10-7.03 (m, 1H), 6.88-6.80 (m, 1H), 6.72-6.62 (m, 1H), 4.95 (d, J=6.0 Hz, 2H), 4.86 (d, J=6.0 Hz, 2H), 3.74 (s, 2H), 3.50 (s, 2H), 2.93 (s, 3H), 2.87-2.74 (m, 2H), 2.09-1.96 (m, 1H), 1.81-1.42 (m, 5H), 0.99-0.78 (m, 4H) ppm.

### Compound B13: 4-{[(3S)-3-methylpiperidin-1-yl]methyl}-N-{3-[(1r, 3s)-3-methyl-1-(4-methyl-1,2,4-triazol-3-yl)cyclobutyl]phenyl}pyrrolo[1,2-b]pyridazine-2-carboxamide

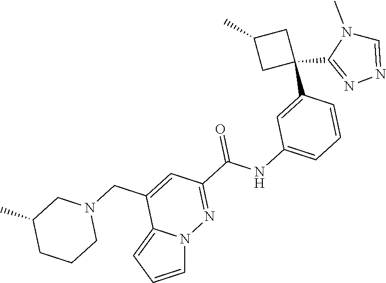 [SMILES: C[C@H]1CCCN(Cc2cc(C(=O)Nc3cccc([C@]4(c5nncn5C)C[C@@H](C)C4)c3)nn3cccc23)C1]

The compound B13 was synthesized starting from the corresponding starting materials according the similar procedures described as those of Compound B12 (23 mg, 54.00%). LC-MS (M+H)⁺:=498. ¹H NMR (300 MHz, DMSO-d6) δ 10.58 (s, 1H), 8.31 (s, 1H), 8.05-7.98 (m, 1H), 7.91-7.83 (m, 2H), 7.42-7.26 (m, 2H), 7.10-7.01 (m, 2H), 6.87-6.80 (m, 1H), 3.74 (s, 2H), 3.19 (s, 3H), 2.89-2.73 (m, 4H), 2.55 (d, J=7.4 Hz, 3H), 2.07-1.96 (m, 1H), 1.80-1.47 (m, 5H), 1.10 (d, J=5.3 Hz, 3H), 0.97-0.79 (m, 4H) ppm.

### Compound B14: 4-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrrolo[1,2-b]pyridazine-2-carboxamide

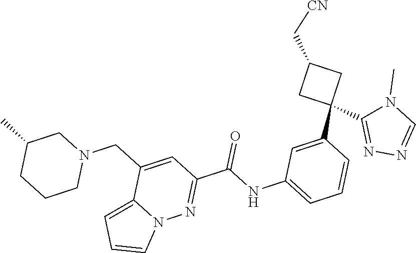 [SMILES: C[C@H]1CCCN(Cc2cc(C(=O)Nc3cccc([C@]4(c5nncn5C)C[C@@H](CC#N)C4)c3)nn3cccc23)C1]

To a stirred mixture of 4-{[(3S)-3-methylpiperidin-1-yl]methyl}pyrrolo[1,2-b]pyridazine-2-carboxylic acid (21 mg, 0.073 mmol, 1.11 equiv, 94.8%) and HOBT (15 mg, 0.105 mmol, 1.60 equiv, 95%), EDCI (20 mg, 0.099 mmol, 1.51 equiv, 95%), NEt3 (21 mg, 0.197 mmol, 2.99 equiv, 95%) in DMF stirred for 5 mins was added 2-[(1s, 3s)-3-(3-aminophenyl)-3-(4-methyl-1,2,4-triazol-3-yl)cyclobutyl]acetonitrile (22 mg, 0.066 mmol, 1.00 equiv, 80%) dropwise at room temperature. The resulting mixture was stirred for 12 h at 40° C. The residue was purified by reverse flash chromatography with the following conditions: column, C18 silica gel; mobile phase, MeCN in Water (0.1% NH3·H2O), 0% to 95% gradient in 15 min; detector, UV 254 nm. The crude product (40 mg) was purified by Prep-HPLC with the following conditions (2 #SHIMADZU (HPLC-01)): Column, XBridge Shield RP18 OBD Column, 30*150 mm, 5 μm; mobile phase, Water (10 mmol/L NH4HCO3+0.1% NH3·H2O) and ACN (42% ACN up to 72% in 8 min); This resulted in 4-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}pyrrolo[1,2-b]pyridazine-2-carboxamide (27.3 mg, 77.44%) as a yellow solid. LC-MS (M+H)⁺:=523; ¹H NMR (400 MHz, DMSO-d6) δ 10.61 (s, 1H), 8.33 (s, 1H), 8.04-7.99 (m, 1H), 7.95-7.88 (m, 1H), 7.90-7.83 (m, 1H), 7.43-7.34 (m, 1H), 7.29 (s, 1H), 7.11-7.02 (m, 2H), 6.86-6.80 (m, 1H), 3.74 (s, 2H), 3.21 (s, 3H), 2.91-2.67 (m, 9H), 2.06-1.96 (m, 1H), 1.77-1.58 (m, 4H), 1.52 (d, J=12.9 Hz, 1H), 0.95-0.79 (m, 4H) ppm.

### Compound B15: 4-(((cyclobutylmethyl)amino)methyl)-N-(5-((1s, 3s)-3-methyl-1-(4-methyl-4H-1,2,4-triazol-3-yl)cyclobutyl)pyridin-3-yl)thieno[2,3-b]pyridine-6-carboxamide

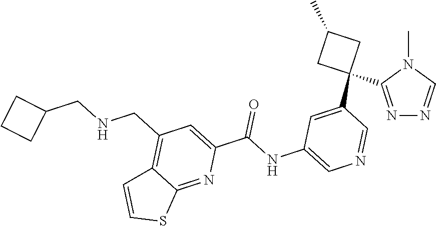 [SMILES: Cn1cnnc1[C@]1(c2cncc(NC(=O)c3cc(CNCC4CCC4)c4ccsc4n3)c2)C[C@H](C)C1]

### Step 1: ethyl 4-[(benzyloxy)methyl]thieno[2,3-b]pyridine-6-carboxylate

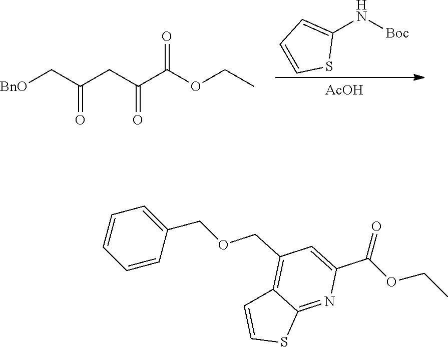

9061 Into a 100 mL sealed tube were added tert-butyl N-(thiophen-2-yl)carbamate (3 g, 14.302 mmol, 1 equiv, 95%) and ethyl 5-(benzyloxy)-2,4-dioxopentanoate (3.9 g, 14.329 mmol, 1.00 equiv, 97.1%) and HOAc (20 mL, 691.073 mmol, 48.32 equiv, 99%) at room temperature. The resulting mixture was stirred for 15 h at 100° C. under nitrogen atmosphere. The mixture was allowed to cool down to room temperature. The resulting mixture was concentrated under vacuum. The residue was purified by silica gel column chromatography, eluted with PE/EA (8:1) to afford ethyl 4-[(benzyloxy)methyl]thieno[2,3-b]pyridine-6-carboxylate (350 mg, 6.70%). MS: M/e 328 (M+1)⁺.

### Step 2: ethyl 4-(hydroxymethyl)thieno[2,3-b]pyridine-6-carboxylate

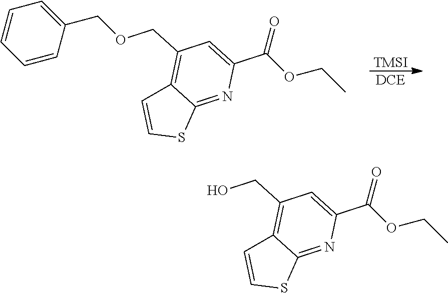

To a stirred solution of ethyl 4-[(benzyloxy)methyl]thieno[2,3-b]pyridine-6-carboxylate (280 mg, 0.767 mmol, 1 equiv, 89.7%) in DCE was added TMSI (1.7 g, 8.071 mmol, 10.52 equiv, 95%) dropwise at 0° C. under nitrogen atmosphere. The resulting mixture was stirred for 15 h at 40° C. under nitrogen atmosphere. The resulting mixture was concentrated under vacuum. The residue was purified by silica gel column chromatography, eluted with PE/EA (5:1) to afford ethyl 4-(hydroxymethyl)thieno[2,3-b]pyridine-6-carboxylate (80 mg, 29.67%). MS: M/e 238 (M+1)⁺.

### Step 3: ethyl 4-(chloromethyl)thieno[2,3-b]pyridine-6-carboxylate

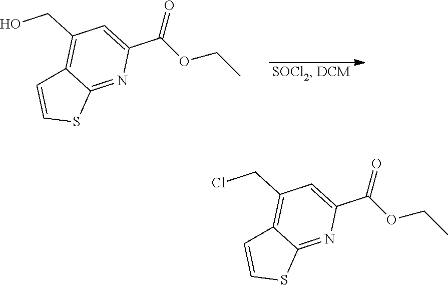

To a stirred solution of ethyl 4-(hydroxymethyl)thieno[2,3-b]pyridine-6-carboxylate (500 mg, 1.937 mmol, 1 equiv, 91.9%) in DCM (15 mL) was added SOCl₂ (2.5 g, 19.965 mmol, 10.31 equiv, 95%) dropwise at 0° C. The resulting mixture was stirred for 3 h at 45° C. under nitrogen atmosphere. The mixture was allowed to cool down to room temperature. The resulting mixture was concentrated under vacuum. The mixture was neutralized to pH 7 with NH₃ in MeOH (7 M). The residue was purified by silica gel column chromatography, eluted with PE/EA (5:1) to afford ethyl 4-(chloromethyl)thieno[2,3-b]pyridine-6-carboxylate (380 mg, 73.20%) as a light yellow solid. LC-MS (M+H)⁺:=256.

### Step 4: ethyl 4-{[(cyclobutylmethyl)amino]methyl}thieno[2,3-b]pyridine-6-carboxylate

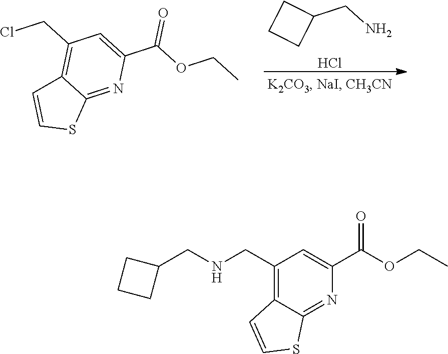 [SMILES: CCOC(=O)c1cc(CCl)c2ccsc2n1 ; NCC1CCC1 ; Cl ; CCOC(=O)c1cc(CNCC2CCC2)c2ccsc2n1]

Into a 10 mL sealed tube were added ethyl 4-(chloromethyl)thieno[2,3-b]pyridine-6-carboxylate (70 mg, 0.261 mmol, 1 equiv, 95.4%), NaI (42 mg, 0.266 mmol, 1.02 equiv, 95%), NaI (42 mg, 0.266 mmol, 1.02 equiv, 95%), K₂CO₃ (76 mg, 0.522 mmol, 2.00 equiv, 95%) and CH₃CN (2 mL, 99%). The resulting mixture was stirred for 3 h at 70° C. under nitrogen atmosphere. The mixture was allowed to cool down to room temperature. The resulting mixture was concentrated under vacuum. The residue was purified by silica gel column chromatography, eluted with PE/EA (1:1) to afford ethyl 4-{[(cyclobutylmethyl)amino]methyl}thieno[2,3-b]pyridine-6-carboxylate (73 mg, 90.91%) as a light yellow solid. LC-MS (M+H)⁺:=305.

### Step 5: 4-{[(cyclobutylmethyl)amino]methyl}thieno[2,3-b]pyridine-6-carboxylic acid

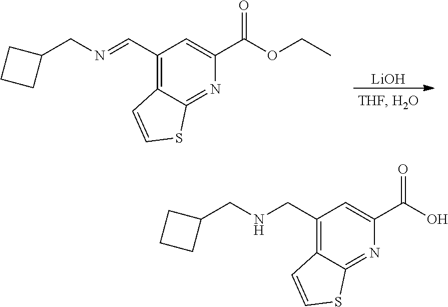

To a stirred solution of ethyl 4-{[(cyclobutylmethyl)amino]methyl}thieno[2,3-b]pyridine-6-carboxylate (72 mg, 0.234 mmol, 1 equiv, 99.0%) in THF (1 mL, 99%) and H₂O (0.5 mL, 99%) were added LiOH (12 mg, 0.476 mmol, 2.03 equiv, 95%). The resulting mixture was stirred for 4 h at room temperature. The mixture was acidified to pH 4 with HCl (1 M). The resulting mixture was concentrated under vacuum. The residue was purified by reversed-phase flash chromatography with the following conditions: column, C18 silica gel; mobile phase, MeCN in Water (0.1% FA), 0% to 95% gradient in 30 min; detector, UV 254 nm. This resulted in 4-{[(cyclobutylmethyl)amino]methyl}thieno[2,3-b]pyridine-6-carboxylic acid (60 mg, 91.79%) as a light yellow solid. LC-MS (M+H)⁺:=277.

### Step 6: 4-{[(cyclobutylmethyl)amino]methyl}-N-{5-[(1r, 3s)-3-methyl-1-(4-methyl-1,2,4-triazol-3-yl)cyclobutyl]pyridin-3-yl}thieno[2,3-b]pyridine-6-carboxamide

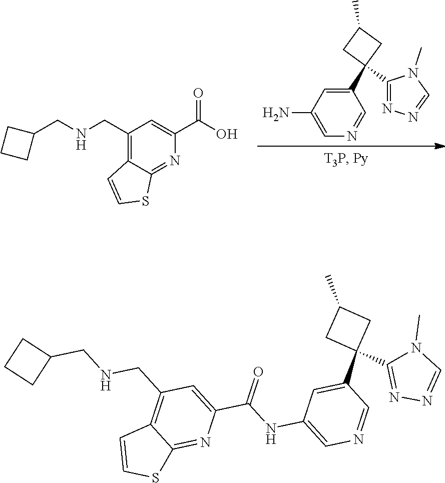 [SMILES: O=C(O)c1cc(CNCC2CCC2)c2ccsc2n1 ; Cn1cnnc1[C@]1(c2cncc(N)c2)C[C@H](C)C1 ; Cn1cnnc1[C@]1(c2cncc(NC(=O)c3cc(CNCC4CCC4)c4ccsc4n3)c2)C[C@H](C)C1]

Into a 10 mL sealed tube were added 4-{[(cyclobutylmethyl)amino]methyl}thieno[2,3-b]pyridine-6-carboxylic acid (25 mg, 0.090 mmol, 1 equiv, 99.0%) and 5-[(1r, 3s)-3-methyl-1-(4-methyl-1,2,4-triazol-3-yl)cyclobutyl]pyridin-3-amine (23 mg, 0.090 mmol, 1.00 equiv, 95.0%) and Pyridine (1 mL, 95%). The resulting mixture was stirred for 5 min at room temperature. To the above mixture was added T₃P (285 mg, 0.448 mmol, 5.00 equiv, 50%) dropwise. The resulting mixture was stirred for additional 3 h at room temperature. The resulting mixture was concentrated under vacuum. The crude product (100 mg) was purified by Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (30% ACN up to 60% in 8 min); Detector, uv product was obtained which Alpha. This resulted in 4-{[(cyclobutylmethyl)amino]methyl}-N-{5-[(1r, 3s)-3-methyl-1-(4-methyl-1,2,4-triazol-3-yl)cyclobutyl]pyridin-3-yl}thieno[2,3-b]pyridine-6-carboxamide (14.1 mg, 30.50%). LC-MS (M+H)⁺:=502. ¹H NMR (300 MHz, DMSO-d₆) δ 10.97 (s, 1H), 9.13 (d, J=2.1 Hz, 1H), 8.33 (s, 1H), 8.32-8.29 (m, 1H), 8.28 (d, J=2.1 Hz, 1H), 8.26 (s, 1H), 8.11 (d, J=6.1 Hz, 1H), 7.73 (d, J=6.1 Hz, 1H), 4.15 (s, 2H), 3.23 (s, 3H), 2.88 (d, J=4.1 Hz, 2H), 2.68-2.54 (m, 5H), 2.47-2.37 (m, 1H), 2.07-1.92 (m, 2H), 1.90-1.73 (m, 2H), 1.73-1.57 (m, 2H), 1.10 (d, J=5.6 Hz, 3H) ppm.

### Compound B16: 3-chloro-N-(3-((1s,3R)-3-(cyanomethyl)-1-(4-methyl-4H-1,2,4-triazol-3-yl)cyclobutyl)phenyl)-7-(((S)-3-methylpiperidin-1-yl)methyl)-1H-pyrrolo[3,2-b]pyridine-5-carboxamide

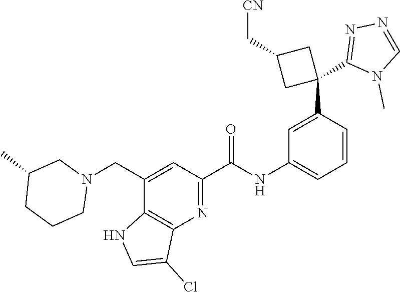 [SMILES: C[C@H]1CCCN(Cc2cc(C(=O)Nc3cccc([C@]4(c5nncn5C)C[C@@H](CC#N)C4)c3)nc3c(Cl)c[nH]c23)C1]

### Step A: methyl (S)-3-chloro-7-((3-methylpiperidin-1-yl)methyl)-1H-pyrrolo[3,2-b]pyridine-5-carboxylate

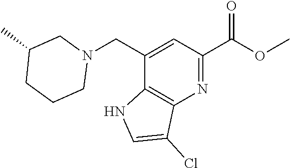

To a solution of methyl (S)-7-((3-methylpiperidin-1-yl)methyl)-1H-pyrrolo[3,2-b]pyridine-5-carboxylate (20 mg, 0.069 mmol) in ACN (3 mL) was added NCS (10 mg, 0.075 mmol). The reaction mixture was stirred for 12 h at 25° C. After completed, the reaction was quenched with aq NH₄Cl (10 mL) and extracted with DCM (3×20 mL), dried over Na₂SO₄ and concentrated under vacuum. The residue was purified by Prep-TLC (DCM/MeOH=20/1) to afford the title compound (20 mg, 91%). MS: M/e 322, 324. (M+H)⁺.

### Step B: (S)-3-chloro-7-((3-methylpiperidin-1-yl)methyl)-1H-pyrrolo[3,2-b]pyridine-5-carboxylic acid

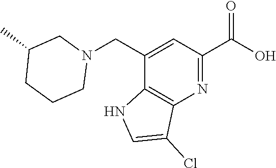 [SMILES: C[C@H]1CCCN(Cc2cc(C(=O)O)nc3c(Cl)c[nH]c23)C1]

To a solution of methyl (S)-3-chloro-7-((3-methylpiperidin-1-yl)methyl)-1H-pyrrolo[3,2-b]pyridine-5-carboxylate (20 mg, 0.062) in MeOH (5 mL)/H₂O (0.5 mL) was added LiOH H₂O (13 mg, 0.309 mmol). The reaction mixture was stirred for 12 h at 25° C. After completed, the solvent was concentrated under vacuum. The residue was diluted with water and neutralized with 2 N HCl and freed dried to afford the title compound (20 mg, crude). MS: M/e 308, 310 (M+H)⁺.

### Step C: 3-chloro-N-(3-((1s,3R)-3-(cyanomethyl)-1-(4-methyl-4H-1,2,4-triazol-3-yl)cyclobutyl)phenyl)-7-(((S)-3-methylpiperidin-1-yl)methyl)-1H-pyrrolo[3,2-b]pyridine-5-carboxamide

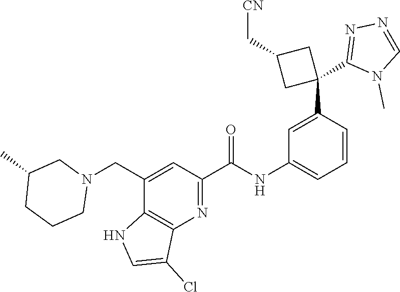 [SMILES: C[C@H]1CCCN(Cc2cc(C(=O)Nc3cccc([C@]4(c5nncn5C)C[C@@H](CC#N)C4)c3)nc3c(Cl)c[nH]c23)C1]

To a solution of (S)-3-chloro-7-((3-methylpiperidin-1-yl)methyl)-1H-pyrrolo[3,2-b]pyridine-5-carboxylic acid (20 mg, crude), 2-((1s, 3s)-3-(3-aminophenyl)-3-(4-methyl-4H-1,2,4-triazol-3-yl)cyclobutyl)acetonitrile (17 mg, 0.063 mmol), DIEA (16 mg, 0.124 mmol) in DMF (2 mL) was added HATU (35 mg, 0.092 mmol). The reaction mixture was stirred for 1 h at 25° C. After completed, the reaction was quenched with aq NH₄Cl (10 mL) and extracted with DCM (3×30 mL), dried over Na₂SO₄ and concentrated under vacuum. The residue was purified by Prep-HPLC to afford the title compound (15 mg, 43%). ¹H NMR (400 MHz, CD₃OD) δ 8.33 (s, 1H), 8.05 (s, 1H), 7.97 (s, 1H), 7.81 (d, J=8.0 Hz, 1H), 7.72 (s, 1H), 7.43 (t, J=8.0 Hz, 1H), 7.13 (d, J=8.0 Hz, 1H), 3.88 (s, 2H), 3.33 (s, 3H), 3.08-3.00 (m, 2H), 3.00-2.90 (m, 1H), 2.89-2.76 (m, 4H), 2.66 (d, J=6.0 Hz, 2H), 2.07-1.95 (m, 1H), 1.79-1.61 (m, 5H), 0.99-0.87 (m, 1H), 0.85 (d, J=5.2 Hz, 3H) ppm. MS: M/e 557, 559 (M+H)⁺.

### Compound B17: N-(3-((1s,3S)-3-(cyanomethyl)-1-(4-methyl-4H-1,2,4-triazol-3-yl)cyclobutyl)phenyl)-7-((((R)-3,3-difluorocyclopentyl)amino)methyl)-1H-pyrrolo[3,2-b]pyridine-5-carboxamide

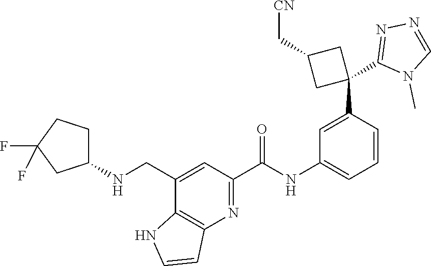 [SMILES: Cn1cnnc1[C@]1(c2cccc(NC(=O)c3cc(CN[C@H]4CCC(F)(F)C4)c4[nH]ccc4n3)c2)C[C@H](CC#N)C1]

### Step A: (S)-7-(((3,3-difluorocyclopentyl)amino)methyl)-1-((2-(trimethylsilyl)ethoxy)methyl)-1H-pyrrolo[3,2-b]pyridine-5-carbonitrile

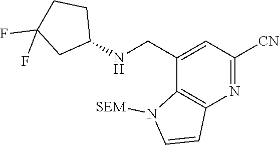 [SMILES: C[Si](C)(C)CCOCn1ccc2nc(C#N)cc(CN[C@H]3CCC(F)(F)C3)c21]

To a solution of 7-formyl-1-((2-(trimethylsilyl)ethoxy)methyl)-1H-pyrrolo[3,2-b]pyridine-5-carbonitrile (50 mg, 0.166 mmol), (S)-3,3-difluorocyclopentan-1-amine hydrochloride (40 mg, 0.253 mmol), Et₃N (25 mg, 0.247 mmol) in DCM (3 mL) was added NaBH(OAc)₃ (70 mg, 0.330 mmol). The reaction mixture was stirred for 12 hrs at 25° C. After completed, the reaction was quenched with aq NaHCO₃ (20 mL) and extracted with DCM (3×20 mL), dried over Na₂SO₄ and concentrated under vacuum. The residue was purified by Prep-TLC (DCM/MeOH=20/1) to afford the title compound (60 mg, 89%). MS: M/e 407 (M+H)⁺.

### Step B: (S)-7-(((3,3-difluorocyclopentyl)amino)methyl)-1-((2-(trimethylsilyl)ethoxy)methyl)-1H-pyrrolo[3,2-b]pyridine-5-carboxylic acid

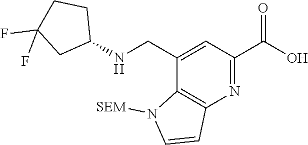 [SMILES: C[Si](C)(C)CCOCn1ccc2nc(C(=O)O)cc(CN[C@H]3CCC(F)(F)C3)c21]

To a solution of (S)-7-(((3,3-difluorocyclopentyl)amino)methyl)-1-((2-(trimethylsilyl)ethoxy)methyl)-1H-pyrrolo[3,2-b]pyridine-5-carbonitrile (60 mg, 0.147 mmol) in EtOH (1 mL) was added NaOH (0.15 mL, 10 M). The reaction mixture was stirred for 12 hrs at 110° C. After completed, the solvent was concentrated under vacuum. The residue was diluted with water and neutralized with 2N HCl and extracted with DCM/MeOH (10/1, 3×30 mL), dried over Na₂SO₄ and concentrated under vacuum. The crude product (70 mg) was obtained as a yellow solid. MS: M/e 426 (M+H)⁺.

### Step C: (S)-7-(((3,3-difluorocyclopentyl)amino)methyl)-1H-pyrrolo[3,2-b]pyridine-5-carboxylic acid

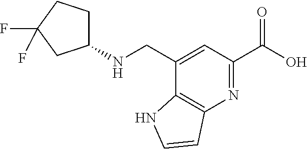 [SMILES: O=C(O)c1cc(CN[C@H]2CCC(F)(F)C2)c2[nH]ccc2n1]

To a solution of (S)-7-(((3,3-difluorocyclopentyl)amino)methyl)-1-((2-(trimethylsilyl)ethoxy)methyl)-1H-pyrrolo[3,2-b]pyridine-5-carboxylic acid (70 mg, crude) in DCM (1 mL) was added TFA (7 mL). The reaction mixture was stirred for 12 hrs at 25° C. After completed, the solvent was removed by in vacuo. The residue was dissolved in MeOH (3 mL) and NH₃H2O (0.1 mL) was added. The reaction mixture was stirred for 2 hrs at 25° C. After completed, the solvent was removed by in vacuo. The residue was purified by Prep-HPLC to afford the title compound (45 mg, impure). MS: M/e 296 (M+H)⁺.

### Step D: N-(3-((1s,3S)-3-(cyanomethyl)-1-(4-methyl-4H-1,2,4-triazol-3-yl)cyclobutyl)phenyl)-7-((((R)-3,3-difluorocyclopentyl)amino)methyl)-1H-pyrrolo[3,2-b]pyridine-5-carboxamide

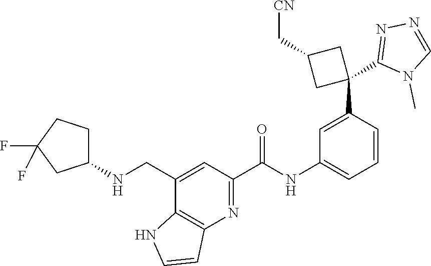 [SMILES: Cn1cnnc1[C@]1(c2cccc(NC(=O)c3cc(CN[C@H]4CCC(F)(F)C4)c4[nH]ccc4n3)c2)C[C@H](CC#N)C1]

To a solution of (S)-7-(((3,3-difluorocyclopentyl)amino)methyl)-1H-pyrrolo[3,2-b]pyridine-5-carboxylic acid (45 mg, crude), 2-((1s, 3s)-3-(3-aminophenyl)-3-(4-methyl-4H-1,2,4-triazol-3-yl)cyclobutyl)acetonitrile (40 mg, 0.149 mmol), DIEA (40 mg, 0.310 mmol) in DMF (2 mL) was added HATU (85 mg, 0.223 mmol). The reaction mixture was stirred for 1 h at 25° C. After completed, the reaction was quenched with aq NH₄Cl (10 mL) and extracted with DCM (3×20 mL), dried over Na₂SO₄ and concentrated under vacuum. The residue was purified by Prep-HPLC to afford the title compound (25 mg, 31%). ¹H NMR (400 MHz, CD₃OD) δ 8.33 (s, 1H), 8.09 (s, 1H), 7.94 (s, 1H), 7.83-7.74 (m, 2H), 7.43 (t, J=7.8 Hz, 1H), 7.13 (d, J=7.8 Hz, 1H), 6.79 (s, 1H), 4.34 (s, 2H), 3.63-3.51 (m, 1H), 3.33 (s, 3H), 3.08-2.80 (m, 5H), 2.70-2.63 (m, 3H), 2.62-2.46 (m, 1H), 2.36-2.20 (m, 2H), 2.18-2.02 (m, 2H), 1.89-1.75 (m, 1H) ppm. MS: M/e 545 (M+H)⁺.

### Compound B18: 4-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}thieno[2,3-b]pyridine-6-carboxamide

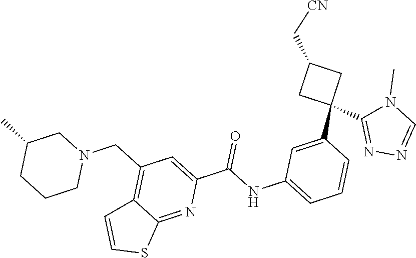 [SMILES: C[C@H]1CCCN(Cc2cc(C(=O)Nc3cccc([C@]4(c5nncn5C)C[C@@H](CC#N)C4)c3)nc3sccc23)C1]

### Step 1: ethyl 4-[(benzyloxy)methyl]thieno[2,3-b]pyridine-6-carboxylate

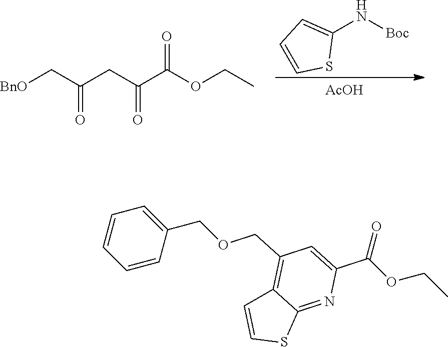

Into a 100 mL sealed tube were added tert-butyl N-(thiophen-2-yl)carbamate (3 g, 14.302 mmol, 1 equiv, 95%) and ethyl 5-(benzyloxy)-2,4-dioxopentanoate (3.9 g, 14.329 mmol, 1.00 equiv, 97.1%) and HOAc (20 mL, 691.073 mmol, 48.32 equiv, 99%) at room temperature. The resulting mixture was stirred for 15 hrs at 100° C. under nitrogen atmosphere. The mixture was allowed to cool down to room temperature. The resulting mixture was concentrated under vacuum. The residue was purified by silica gel column chromatography, eluted with PE/EA (8:1) to afford ethyl 4-[(benzyloxy)methyl]thieno[2,3-b]pyridine-6-carboxylate (350 mg, 6.70%).

### Step 2: ethyl 4-(hydroxymethyl)thieno[2,3-b]pyridine-6-carboxylate

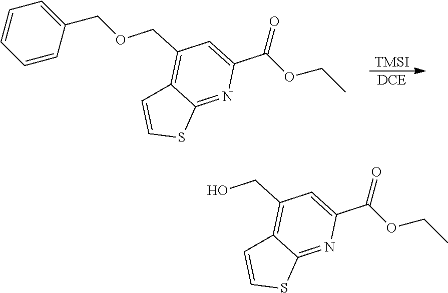

To a stirred solution of ethyl 4-[(benzyloxy)methyl]thieno[2,3-b]pyridine-6-carboxylate (280 mg, 0.767 mmol, 1 equiv, 89.7%) in DCE was added TMSI (1.7 g, 8.071 mmol, 10.52 equiv, 95%) dropwise at 0° C. under nitrogen atmosphere. The resulting mixture was stirred for 15 hrs at 40° C. under nitrogen atmosphere. The resulting mixture was concentrated under vacuum. The residue was purified by silica gel column chromatography, eluted with PE/EA (5:1) to afford ethyl 4-(hydroxymethyl)thieno[2,3-b]pyridine-6-carboxylate (80 mg, 29.67%). MS: M/e 238 (M+1)⁺.

### Step 3: ethyl 4-formylthieno[2,3-b]pyridine-6-carboxylate

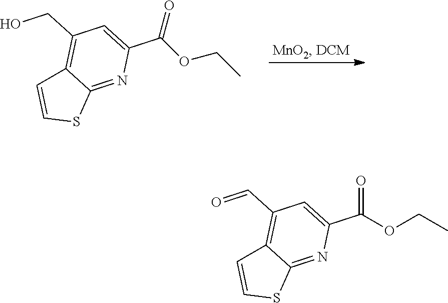

To a stirred solution of ethyl 4-(hydroxymethyl)thieno[2,3-b]pyridine-6-carboxylate (60 mg, 0.171 mmol, 1 equiv, 67.5%) in DCM was added MnO₂ (125 mg, 1.366 mmol, 8.00 equiv, 95%) in portions at room temperature. The resulting mixture was stirred for 14 hrs at 40° C. The residue was purified by silica gel column chromatography, eluted with PE/EA (4:1) to afford ethyl 4-formylthieno[2,3-b]pyridine-6-carboxylate (24 mg, 43.75%). MS: M/e 236 (M+1)⁺

### Step 4: ethyl 4-{[(3S)-3-methylpiperidin-1-yl]methyl}thieno[2,3-b]pyridine-6-carboxylate

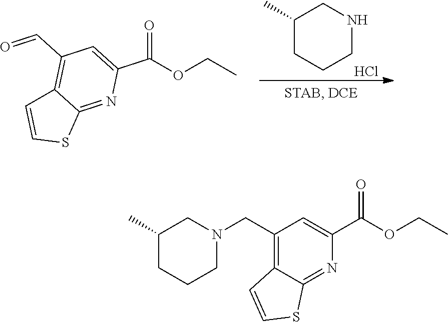

Into a 10 mL sealed tube were added ethyl 4-formylthieno[2,3-b]pyridine-6-carboxylate (20 mg, 0.062 mmol, 1 equiv, 73.2%) and (3S)-3-methylpiperidine hydrochloride (20 mg, 0.140 mmol, 2.25 equiv, 95%) at room temperature. The resulting mixture was stirred for 4 hrs at 60° C. under nitrogen atmosphere. The mixture was allowed to cool down to room temperature. To the above mixture was added STAB (20 mg, 0.090 mmol, 1.44 equiv, 95%) in portions at room temperature. The resulting mixture was stirred for additional 3 hrs at 40° C. The resulting mixture was concentrated under vacuum. The residue was purified by silica gel column chromatography, eluted with PE/EA (3:7) to afford ethyl 4-{[(3S)-3-methylpiperidin-1-yl]methyl}thieno[2,3-b]pyridine-6-carboxylate (16 mg, 60.39%). MS: M/e 319 (M+1)⁺

### Step 5: 4-{[(3S)-3-methylpiperidin-1-yl]methyl}thieno[2,3-b]pyridine-6-carboxylic acid

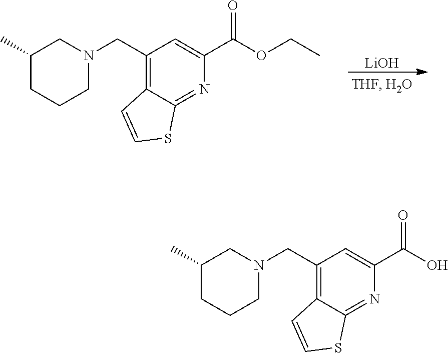

To a stirred solution of ethyl 4-{[(3S)-3-methylpiperidin-1-yl]methyl}thieno[2,3-b]pyridine-6-carboxylate (16 mg, 0.037 mmol, 1 equiv, 73.2%) in THF and H₂O (0.5 mL, 99%) was added LiOH (2 mg, 0.079 mmol, 2.16 equiv, 95%). The resulting mixture was stirred for 4 hrs at room temperature. The resulting mixture was concentrated under vacuum. The residue was purified by reverse flash chromatography with the following conditions: column, C18 silica gel; mobile phase, MeCN in Water (0.1% NH₃·H₂O), 0% to 50% gradient in 15 min; detector, UV 220 nm. This resulted in 4-{[(3S)-3-methylpiperidin-1-yl]methyl}thieno[2,3-b]pyridine-6-carboxylic acid (7 mg, 65.48%). MS: M/e 291 (M+1)⁺

### Step 6:4-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}thieno[2,3-b]pyridine-6-carboxamide

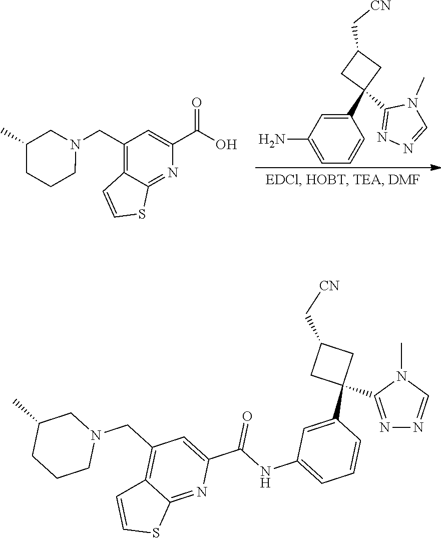 [SMILES: C[C@H]1CCCN(Cc2cc(C(=O)O)nc3sccc23)C1 ; Cn1cnnc1[C@]1(c2cccc(N)c2)C[C@H](CC#N)C1 ; C[C@H]1CCCN(Cc2cc(C(=O)Nc3cccc([C@]4(c5nncn5C)C[C@@H](CC#N)C4)c3)nc3sccc23)C1]

To a stirred mixture of 4-{[(3S)-3-methylpiperidin-1-yl]methyl}thieno[2,3-b]pyridine-6-carboxylic acid (7 mg, 0.024 mmol, 1 equiv, 99.9%) and HOBT (6 mg, 0.042 mmol, 1.75 equiv, 95%) and EDC·HCl (8 mg, 0.040 mmol, 1.65 equiv, 95%) in DMF was added Et₃N (8 mg, 0.075 mmol, 3.12 equiv, 95%). stirred for 10 mins, To the above mixture was added 2-[(1s, 3s)-3-(3-aminophenyl)-3-(4-methyl-1,2,4-triazol-3-yl)cyclobutyl]acetonitrile (8 mg, 0.024 mmol, 0.99 equiv, 80.0%) in DMF dropwise. The resulting mixture was stirred for additional 12 hrs at 40° C. The crude product (15 mg) was purified by Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH3·H2O) and ACN (40% ACN up to 70% in 68 min); Detector, This resulted in 4-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}thieno[2,3-b]pyridine-6-carboxamide (7.2 mg, 55.01%). MS: M/e 540 (M+1)⁺. ¹H NMR (300 MHz, DMSO-d₆) δ 10.72 (s, 1H), 8.26 (s, 1H), 8.12-8.00 (m, 2H), 7.95-7.87 (m, 2H), 7.74-7.66 (m, 1H), 7.36-7.25 (m, 1H), 6.95 (d, J=7.8 Hz, 1H), 3.82 (s, 2H), 3.15 (s, 3H), 2.87-2.60 (m, 9H), 2.00-1.87 (m, 1H), 1.73-1.35 (m, 5H), 0.86-0.71 (m, 4H) ppm.

### Compound B19: N-(3-((1s,3S)-3-(cyanomethyl)-1-(4-methyl-4H-1,2,4-triazol-3-yl)cyclobutyl)phenyl)-7-(((3R,5S)-4,4-difluoro-3,5-dimethylpiperidin-1-yl)methyl)-1H-pyrrolo[3,2-b]pyridine-5-carboxamide

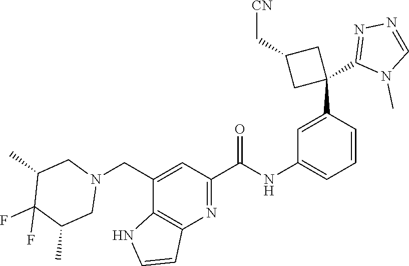 [SMILES: C[C@@H]1CN(Cc2cc(C(=O)Nc3cccc([C@]4(c5nncn5C)C[C@@H](CC#N)C4)c3)nc3cc[nH]c23)C[C@H](C)C1(F)F]

### Step A: methyl 7-(((3R,5S)-4,4-difluoro-3,5-dimethylpiperidin-1-yl)methyl)-1-((2-(trimethylsilyl)ethoxy)methyl)-1H-pyrrolo[3,2-b]pyridine-5-carboxylate

To a solution of methyl 7-formyl-1-((2-(trimethylsilyl)ethoxy)methyl)-1H-pyrrolo[3,2-b]pyridine-5-carboxylate (50 mg, 0.15 mmol), (3S,5R)-4,4-difluoro-3,5-dimethylpiperidine (34 mg, 0.228 mmol), AcOH (20 uL) in DCM (3 mL) was added NaBH(OAc)₃ (64 mg, 0.301 mmol). The reaction mixture was stirred for 12 h at 25° C. After completed, the reaction was quenched with aq NaHCO₃ (20 mL) and extracted with DCM (3×20 mL), dried over Na₂SO₄ and concentrated under vacuum. The residue was purified by Prep-TLC (DCM/MeOH=20/1) to afford the title compound (60 mg, impure). MS: M/e 468. (M+H)⁺.

### Step B: methyl 7-(((3R,5S)-4,4-difluoro-3,5-dimethylpiperidin-1-yl)methyl)-1H-pyrrolo[3,2-b]pyridine-5-carboxylate

To a solution of methyl 7-(((3R,5S)-4,4-difluoro-3,5-dimethylpiperidin-1-yl)methyl)-1-((2-(trimethylsilyl)ethoxy)methyl)-1H-pyrrolo[3,2-b]pyridine-5-carboxylate (60 mg, impure) in DCM (0.5 mL) was added TFA (2.5 mL). The reaction mixture was stirred for 24 h at 25° C. After completed, the solvent was removed by in vacuo. The residue was dissolved in MeOH (3 mL) and Et₃N (0.2 mL) was added. The reaction mixture was stirred for 12 h at 25° C. After completed, the solvent was removed by in vacuo. The residue was purified by Prep-TLC (DCM/MeOH=15/1) to afford the title compound (32 mg, impure). MS: M/e 338. (M+H)⁺.

### Step C: 7-(((3R,5S)-4,4-difluoro-3,5-dimethylpiperidin-1-yl)methyl)-1H-pyrrolo[3,2-b]pyridine-5-carboxylic acid

To a solution of methyl 7-(((3R,5S)-4,4-difluoro-3,5-dimethylpiperidin-1-yl)methyl)-1H-pyrrolo[3,2-b]pyridine-5-carboxylate (32 mg, impure) in MeOH (5 mL)/H₂O (0.5 mL) was added LiOH H₂O (20 mg, 0.476 mmol). The reaction mixture was stirred for 12 h at 25° C. After completed, the solvent was concentrated under vacuum. The residue was diluted with water and neutralized with 2 N HCl and freed dried to afford the title compound (30 mg, crude). MS: M/e 324. (M+H)⁺.

### Step D: N-(3-((1s,3S)-3-(cyanomethyl)-1-(4-methyl-4H-1,2,4-triazol-3-yl)cyclobutyl)phenyl)-7-(((3R,5S)-4,4-difluoro-3,5-dimethylpiperidin-1-yl)methyl)-1H-pyrrolo[3,2-b]pyridine-5-carboxamide

To a solution of 7-(((3R,5S)-4,4-difluoro-3,5-dimethylpiperidin-1-yl)methyl)-1H-pyrrolo[3,2-b]pyridine-5-carboxylic acid (30 mg, crude), 2-((1s, 3s)-3-(3-aminophenyl)-3-(4-methyl-4H-1,2,4-triazol-3-yl)cyclobutyl)acetonitrile (25 mg, 0.093 mmol), DIEA (24 mg, 0.186 mmol) in DMF (2 mL) was added HATU (53 mg, 0.139 mmol). The reaction mixture was stirred for 1 h at 25° C. After completed, the reaction was quenched with aq NH₄Cl (10 mL) and extracted with DCM (3×20 mL), dried over Na₂SO₄ and concentrated under vacuum. The residue was purified by prep-HPLC to afford the title compound (25 mg, 46%). ¹H NMR (400 MHz, DMSO-d₆) δ 11.50 (s, 1H), 10.62 (s, 1H), 8.32 (s, 1H), 7.96 (s, 2H), 7.91 (s, 1H), 7.80 (s, 1H), 7.36 (t, J=8.0 Hz, 1H), 6.98 (d, J=8.0 Hz, 1H), 6.77 (s, 1H), 3.92 (s, 2H), 3.22 (s, 3H), 2.90-2.81 (m, 4H), 2.80-2.68 (m, 5H), 2.34-2.14 (m, 2H), 2.02 (t, J=11.4 Hz, 2H), 0.90 (d, J=6.8 Hz, 6H) ppm. MS: M/e 573 (M+H)⁺.

### Compound B20: N-(3-((1r,3S)-3-cyano-1-((4-methyl-4H-1,2,4-triazol-3-yl)methyl)cyclobutyl)phenyl)-3-fluoro-7-(((S)-3-methylpiperidin-1-yl)methyl)-1H-pyrrolo[3,2-b]pyridine-5-carboxamide

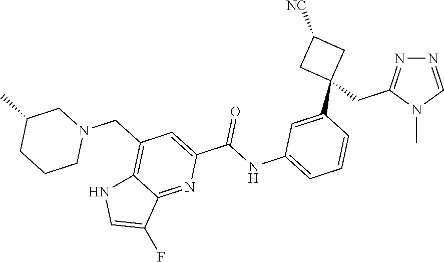 [SMILES: C[C@H]1CCCN(Cc2cc(C(=O)Nc3cccc([C@]4(Cc5nncn5C)C[C@@H](C#N)C4)c3)nc3c(F)c[nH]c23)C1]

To a solution of (S)-3-fluoro-7-((3-methylpiperidin-1-yl)methyl)-1H-pyrrolo[3,2-b]pyridine-5-carboxylic acid (60 mg, 0.206 mmol), (1r, 3r)-3-(3-aminophenyl)-3-((4-methyl-4H-1,2,4-triazol-3-yl)methyl)cyclobutane-1-carbonitrile (55 mg, 0.206 mmol), DIPEA (53 mg, 0.412 mmol) in DMF (5 mL) was added HATU (117 mg, 0.309 mmol). The reaction mixture was stirred for 4 hrs at 25° C. After completed, the reaction was quenched with aq NH₄Cl (10 mL) and extracted with DCM (3×30 mL), dried over Na₂SO₄ and concentrated under vacuum. The residue was purified by Prep-HPLC to afford the title compound (29 mg, 26%). MS: M/e 541 (M+H)⁺. ¹H NMR (400 MHz, CD₃OD) δ 8.17 (s, 1H), 8.05 (s, 1H), 7.74 (d, J=7.9 Hz, 1H), 7.60 (s, 1H), 7.54 (s, 1H), 7.31 (t, J=7.7 Hz, 1H), 6.77 (d, J=7.6 Hz, 1H), 4.02 (s, 2H), 3.41 (s, 2H), 3.22-3.16 (m, 1H), 3.03-2.91 (m, 6H), 2.81 (s, 3H), 2.23-2.17 (m, 1H), 1.90 (t, J=10.6 Hz, 1H), 1.83-1.65 (m, 4H), 1.03-0.97 (m, 1H), 0.88 (d, J=5.6 Hz, 3H) ppm.

### Compound B21:7-((isobutylamino)methyl)-3-methyl-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

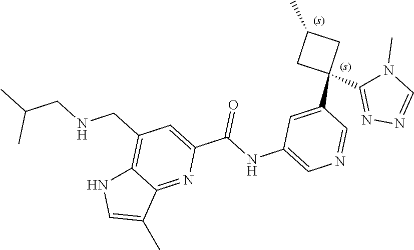

### Step A: 7-chloro-3-iodo-1H-pyrrolo[3,2-b]pyridine-5-carbonitrile

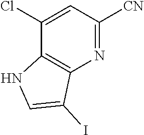 [SMILES: N#Cc1cc(Cl)c2[nH]cc(I)c2n1]

To a solution of 7-chloro-1H-pyrrolo[3,2-b]pyridine-5-carbonitrile (2.0 g, 11.23 mmol) in CH₃CN (40 mL) at 0° C. was added NIS (2.78 g, 12.36 mmol). The reaction mixture was stirred at room temperature for 6 hours. After completed, the reaction solution was concentrated under vacuum. The residue was washed with H₂O (30 mL×2) and free-dried to give the titled compound (3.25 g, 95%). MS: M/e 304 (M+1)⁺

### Step B: 7-chloro-3-iodo-1-((2-(trimethylsilyl)ethoxy)methyl)-1H-pyrrolo[3,2-b]pyridine-5-carbonitrile

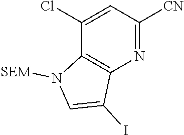 [SMILES: C[Si](C)(C)CCOCn1cc(I)c2nc(C#N)cc(Cl)c21]

To a solution of 7-chloro-3-iodo-1H-pyrrolo[3,2-b]pyridine-5-carbonitrile (3.25 g, 10.726 mmol) in THF (50 mL) at 0° C. was added NaH (644 mg, 16.089 mmol, 60%). The mixture was stirred at 0° C. for 15 mins, then added SEMCl (2.69 g, 16.089 mmol). The reaction mixture was stirred at room temperature for 12 hours. After completed, the reaction solution was quenched with saturated NH₄Cl aq. (30 mL), extracted with EA (50 mL×2), combined, washed brine (30 mL×2), dried and concentrated to dryness. The resulting residue was purified by flash column chromatography (PE:EA=3:1) to give the titled compound (4.3 g, 93%). MS: M/e 434 (M+1)⁺

### Step C: 7-chloro-3-methyl-1-((2-(trimethylsilyl)ethoxy)methyl)-1H-pyrrolo[3,2-b]pyridine-5-carbonitrile

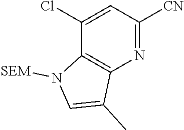 [SMILES: Cc1cn(COCC[Si](C)(C)C)c2c(Cl)cc(C#N)nc12]

To a solution of 7-chloro-3-iodo-1-((2-(trimethylsilyl)ethoxy)methyl)-1H-pyrrolo[3,2-b]pyridine-5-carbonitrile (4.21 g, 9.723 mmol), tetramethylstannane (2.263 g, 12.640 mmol), PdCl₂(PPh₃)₂(682 mg, 0.972 mmol) and LiCl (1.225 g, 2.9.17 mmol) in DMF (50 mL) was degassed 3 times under N₂ atmosphere. The mixture was stirred at 105° C. for 12 hours. The reaction mixture was quenched with saturated NaCl aq. (20 mL), extracted with EA (50 mL×2), combined, washed brine (30 mL×2), dried and concentrated to dryness. The resulting residue was purified by flash column chromatography (PE:EA=4:1) to give the titled compound (2.05 g, 65%). MS: M/e 322 (M+1)⁺

### Step D: 7-chloro-3-methyl-1-((2-(trimethylsilyl)ethoxy)methyl)-1H-pyrrolo[3,2-b]pyridine-5-carboxylic acid

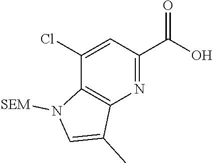 [SMILES: Cc1cn(COCC[Si](C)(C)C)c2c(Cl)cc(C(=O)O)nc12]

To a solution of 7-chloro-3-methyl-1-((2-(trimethylsilyl)ethoxy)methyl)-1H-pyrrolo[3,2-b]pyridine-5-carbonitrile (500 mg, 1.558 mmol) in MeOH (2 mL) was added NaOH (1.6 mL, 15.58 mmol, 10 N). The reaction mixture was stirred at 105° C. for 16 hours. After completed, the solvent was concentrated under vacuum to give the crude product. Added 2N HCl to adjust PH-7 and extracted with DCM/MeOH 10:1 (30 mL×2). The organic layers were dried over Na₂SO₄ and concentrated under vacuum to give the titled compound (450 mg, 85%). MS: M/e 341 (M+1)⁺

### Step E: 7-chloro-3-methyl-N-(5-((1s, 3s)-3-methyl-1-(4-methyl-4H-1,2,4-triazol-3-yl)cyclobutyl)pyridin-3-yl)-1-((2-(trimethylsilyl)ethoxy)methyl)-1H-pyrrolo[3,2-b]pyridine-5-carboxamide

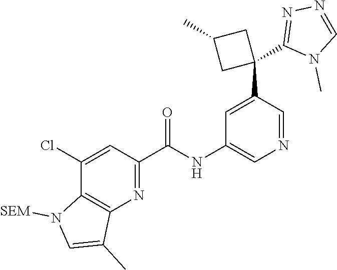 [SMILES: Cc1cn(COCC[Si](C)(C)C)c2c(Cl)cc(C(=O)Nc3cncc([C@]4(c5nncn5C)C[C@@H](C)C4)c3)nc12]

To a solution of 7-chloro-3-methyl-1-((2-(trimethylsilyl)ethoxy)methyl)-1H-pyrrolo[3,2-b]pyridine-5-carboxylic acid (280 mg, 0.823 mmol), 5-((1s, 3s)-3-methyl-1-(4-methyl-4H-1,2,4-triazol-3-yl)cyclobutyl)pyridin-3-amine (200 mg, 0.823 mmol), DIPEA (319 mg, 2.469 mmol) in DMF (30 mL) was added HATU (626 mg, 1.646 mmol). The reaction mixture was stirred for 6 h at 25° C. After completed, the reaction was quenched with aq NH₄Cl (10 mL) and extracted with EA (2×45 mL), dried over Na₂SO₄ and concentrated under vacuum. The residue was purified by flash column chromatography (DCM:MeOH=20:1) to give the titled compound (440 mg, 95%). MS: M/e 566 (M+1)⁺

### Step F: 3-methyl-N-(5-((1s, 3s)-3-methyl-1-(4-methyl-4H-1,2,4-triazol-3-yl)cyclobutyl)pyridin-3-yl)-1-((2-(trimethylsilyl)ethoxy)methyl)-7-vinyl-1H-pyrrolo[3,2-b]pyridine-5-carboxamide

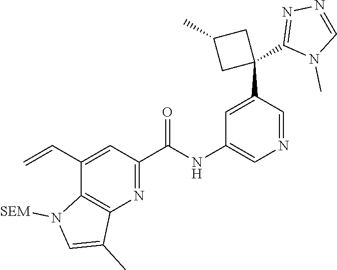 [SMILES: C=Cc1cc(C(=O)Nc2cncc([C@]3(c4nncn4C)C[C@@H](C)C3)c2)nc2c(C)cn(COCC[Si](C)(C)C)c12]

To a solution of 7-chloro-3-methyl-N-(5-((1s, 3s)-3-methyl-1-(4-methyl-4H-1,2,4-triazol-3-yl)cyclobutyl)pyridin-3-yl)-1-((2-(trimethylsilyl)ethoxy)methyl)-1H-pyrrolo[3,2-b]pyridine-5-carboxamide (440 mg, 0.779 mmol), potassium trifluoro(vinyl)borate (136 mg, 1.012 mmol), XPhos G2 Pd (61 mg, 0.078 mmol) and Cs₂CO₃ (508 mg, 1.558 mmol) in dioxane (35 mL) and H₂O (3 mL) was stirred at 80° C. under N₂ for 12 hours. The reaction mixture was quenched with saturated NaCl aq. (20 mL), extracted with EA (45 mL×2), combined, washed brine (30 mL×2), dried and concentrated to dryness. The resulting residue was purified by flash column chromatography (DCM:MeOH=20:1) to give the titled compound (380 mg, 88%). MS: M/e 558 (M+1)⁺

### Step G: 3-methyl-N-(5-((1s, 3s)-3-methyl-1-(4-methyl-4H-1,2,4-triazol-3-yl)cyclobutyl)pyridin-3-yl)-7-vinyl-1H-pyrrolo[3,2-b]pyridine-5-carboxamide

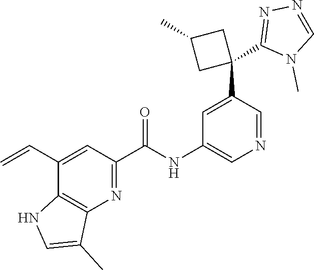 [SMILES: C=Cc1cc(C(=O)Nc2cncc([C@]3(c4nncn4C)C[C@@H](C)C3)c2)nc2c(C)c[nH]c12]

To a solution of 3-methyl-N-(5-((1s, 3s)-3-methyl-1-(4-methyl-4H-1,2,4-triazol-3-yl)cyclobutyl)pyridin-3-yl)-1-((2-(trimethylsilyl)ethoxy)methyl)-7-vinyl-1H-pyrrolo[3,2-b]pyridine-5-carboxamide (380 mg, 0.682 mmol) in DCM (2 mL) was added TFA (5 mL). The reaction mixture was stirred at room temperature for 16 hours. After completed, the solvent was concentrated under vacuum to give the intermediate. The intermediate was stirred at NH₃H2O (5 mL) and MeOH (3 mL) at room temperature for 2 hours. Then the reaction solution was concentrated under vacuum to give the crude product, which was purified by flash column chromatography (DCM:MeOH=15:1) to give the titled compound (290 mg, crude). MS: M/e 428 (M+1)⁺

### Step H: 7-formyl-3-methyl-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

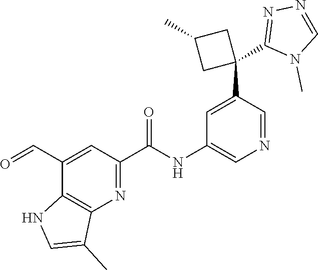 [SMILES: Cc1c[nH]c2c(C=O)cc(C(=O)Nc3cncc([C@]4(c5nncn5C)C[C@@H](C)C4)c3)nc12]

To a solution of 3-methyl-N-(5-((1s, 3s)-3-methyl-1-(4-methyl-4H-1,2,4-triazol-3-yl)cyclobutyl)pyridin-3-yl)-7-vinyl-1H-pyrrolo[3,2-b]pyridine-5-carboxamide (290 mg, 0.679 mmol), K₂OsO₄ 2H₂O (25 mg, 0.068 mmol) in dioxane (30 mL) and H₂O (4 mL) at 0° C. was added NaIO₄ (436 mg, 2.037 mmol). The mixture was stirred at room temperature for 6 hours. After filtration, the solution was concentrated to dryness. The resulting residue was purified by flash column chromatography (DCM:MeOH=15:1) to give the titled compound (100 mg, 34%). MS: M/e 430 (M+1)⁺

### Step I: 7-((isobutylamino)methyl)-3-methyl-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

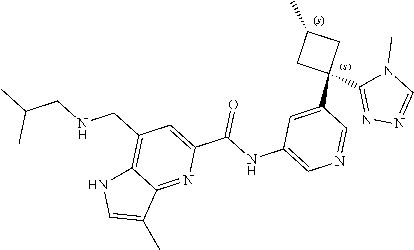

To a solution of 7-formyl-3-methyl-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide (30 mg, 0.070 mmol) and 2-methylpropan-1-amine (10 mg, 0.140 mmol) in DCM (1 ml) was stirred at room temperature for 1 hour, then added NaBH(OAc)₃ (59 mg, 0.280 mmol). The mixture solution was stirred at 25° C. for 24 hours. The reaction mixture was concentrated under reduced pressure. The crude product was purified by Prep-HPLC to give the title compound (11 mg, 32%). MS: M/e 487 (M+1). ¹H NMR (400 MHz, DMSO) δ 10.64 (s, 1H), 9.06 (s, 1H), 8.33 (s, 1H), 8.26 (s, 2H), 7.99 (s, 1H), 7.57 (s, 1H), 4.06 (s, 2H), 3.22 (s, 3H), 2.88 (d, J=3.9 Hz, 2H), 2.63-2.52 (m, 3H), 2.42 (s, 3H), 2.33 (d, J=6.7 Hz, 2H), 1.71 (dt, J=13.3, 6.7 Hz, 1H), 1.08 (d, J=5.4 Hz, 3H), 0.86 (d, J=6.6 Hz, 6H) ppm.

### Compound B22: N-(3-((1s, 3s)-3-(cyanomethyl)-1-(4-methyl-4H-1,2,4-triazol-3-yl)cyclobutyl)phenyl)-7-((((4,4-difluorocyclohexyl)methyl)amino)methyl)-1H-pyrrolo[3,2-b]pyridine-5-carboxamide

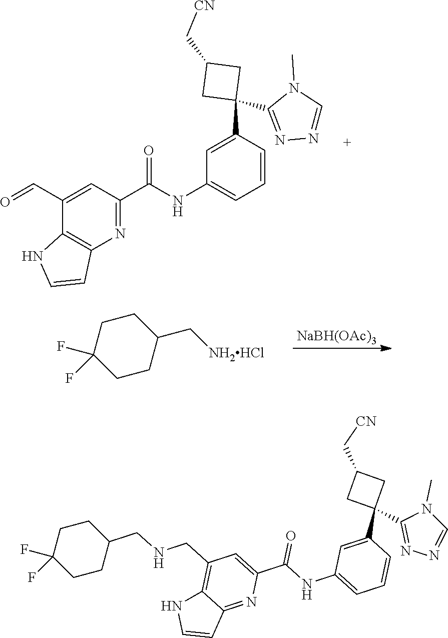

### Step A: N-(3-((1s, 3s)-3-(cyanomethyl)-1-(4-methyl-4H-1,2,4-triazol-3-yl) cyclobutyl)phenyl)-7-vinyl-1H-pyrrolo[3,2-b]pyridine-5-carboxamide

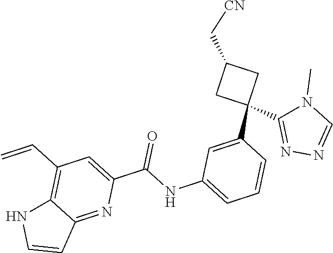 [SMILES: C=Cc1cc(C(=O)Nc2cccc([C@]3(c4nncn4C)C[C@@H](CC#N)C3)c2)nc2cc[nH]c12]

A solution of 7-vinyl-1H-pyrrolo[3,2-b]pyridine-5-carboxylic acid (2 g, 8 mmol) and 2-((1s, 3s)-3-(3-aminophenyl)-3-(4-methyl-4H-1,2,4-triazol-3-yl)cyclo butyl)acetonitrile (2.1 g, 8 mmol) in DMF (25 mL) were added with HATU (4.5 g, 12 mmol) and DIEA (2.1 g, 16 mmol). The reaction mixture was stirred at r.t for 2 hrs, then added with water (15 mL), extracted with DCM (20 mL) and washed with brine (20 mL). The organic layer was dried, concentrated and purified by CombiFlash (DCM:MeOH=6%) to get the product (2.4 g, 69%). MS: M/e 438 (M+1)⁺

### Step B: N-(3-((1s, 3s)-3-(cyanomethyl)-1-(4-methyl-4H-1,2,4-triazol-3-yl)cyclo butyl)phenyl)-7-formyl-1H-pyrrolo[3,2-b]pyridine-5-carboxamide

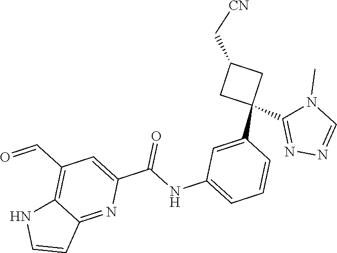 [SMILES: Cn1cnnc1[C@]1(c2cccc(NC(=O)c3cc(C=O)c4[nH]ccc4n3)c2)C[C@H](CC#N)C1]

To a solution of N-(3-((1s, 3s)-3-(cyanomethyl)-1-(4-methyl-4H-1,2,4-triazol-3-yl)cyclobutyl)phenyl)-7-vinyl-1H-pyrrolo[3,2-b]pyridine-5-carboxamide (2.4 g, 5.5 mmol) and K₂Os₄·2H₂O (202 mg, 0.55 mmol) in dioxane (40 mL) and water (8 mL) was cooled under ice bath. NaIO₄ (3.5 g, 16.5 mmol) was added in portionwise. Then it was stirred at r.t for 3 hrs. The thickness suspension was filtered and washed with methanol (5 mL). The filtrate was concentrated to get the crude product, which was purified by CombiFlash (DCM:MeOH=5%) to get the pure product (1.4 g, 58%). MS: M/e 440 (M+1)⁺

### Step C: N-(3-((1s, 3s)-3-(cyanomethyl)-1-(4-methyl-4H-1,2,4-triazol-3-yl)cyclobutyl)phenyl)-7-((((4,4-difluorocyclohexyl)methyl)amino)methyl)-1H-pyrrolo[3,2-b]pyridine-5-carboxamide

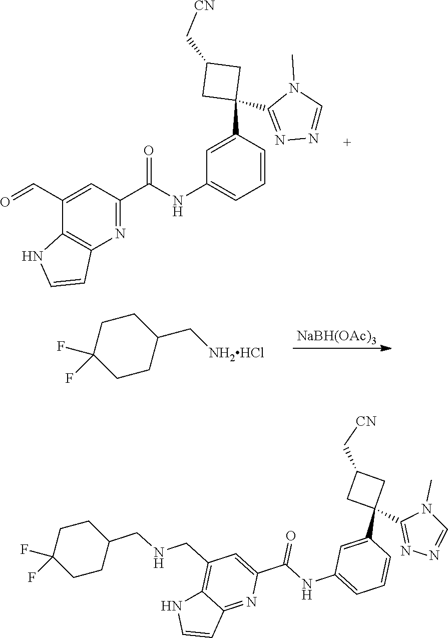

A solution of N-(3-((1s, 3s)-3-(cyanomethyl)-1-(4-methyl-4H-1,2,4-triazol-3-yl) cyclobutyl)phenyl)-7-formyl-1H-pyrrolo[3,2-b]pyridine-5-carboxamide (50 mg, 0.11 mmol), (4,4-difluorocyclohexyl)methanamine hydrochloride (41 mg, 0.22 mmol) and TEA (22 mg, 0.22 mmol) in DCM (2 mL) was stirred at r.tRT for 10 mins. Then it was cooled under ice bath, and NaBH(OAc)₃ (47 mg, 0.22 mmol) was added. The reaction mixture was stirred at r.tRT for 6 hrs. More of NaBH(OAc)₃ (47 mg, 0.22 mmol) was added and it continued to stir overnight. The reaction mixture was added with water (3 mL), basified with NaHCO₃ solution to pH=7-8 and extracted with DCM (6 mL). The organic layer was dried, concentrated and purified by Prep-prep.TLC (DCM:MeOH=8:1) to get the product (32 mg, 49%). ¹H NMR (400 MHz, CD₃OD) δ 8.34 (s, 1H), 8.13 (s, 1H), 7.93 (s, 1H), 7.80 (s, 1H), 7.79 (d, J=8.0 Hz, 1H), 7.43 (t, J=8.0 Hz, 1H), 7.15 (d, J=8.0 Hz, 1H), 6.82 (s, 1H), 4.50 (s, 2H), 3.33 (s, 3H), 3.02 (d, J=8.0 Hz, 2H), 2.93 (s, 3H), 2.89-2.84 (m, 2H), 2.66 (d, J=8.0 Hz, 2H), 2.06 (s, 2H), 1.93-1.72 (m, 5H), 1.39-1.29 (m, 2H) ppm. MS: M/e 573 (M+1)⁺

### Compound B23: N-(3-((1r, 3r)-3-cyano-1-((4-methyl-4H-1,2,4-triazol-3-yl)methyl)cyclobutyl)phenyl)-7-((((1-methylcyclopentyl)methyl)amino)methyl)-1H-pyrrolo[3,2-b]pyridine-5-carboxamide

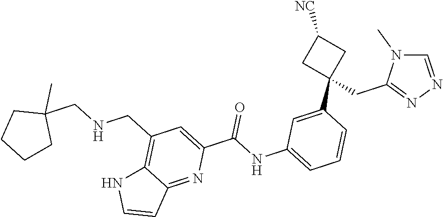 [SMILES: Cn1cnnc1C[C@]1(c2cccc(NC(=O)c3cc(CNCC4(C)CCCC4)c4[nH]ccc4n3)c2)C[C@H](C#N)C1]

### Step A: N-(3-((1r, 3r)-3-cyano-1-((4-methyl-4H-1,2,4-triazol-3-yl)methyl)cyclobutyl) phenyl)-7-vinyl-1H-pyrrolo[3,2-b]pyridine-5-carboxamide

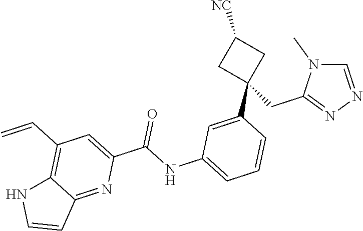 [SMILES: C=Cc1cc(C(=O)Nc2cccc([C@]3(Cc4nncn4C)C[C@@H](C#N)C3)c2)nc2cc[nH]c12]

A solution of 7-vinyl-1H-pyrrolo[3,2-b]pyridine-5-carboxylic acid (0.75 g, 4 mmol) and (1r, 3r)-3-(3-aminophenyl)-3-((4-methyl-4H-1,2,4-triazol-3-yl)methyl) cyclobutane-1-carbonitrile (1 g, 4 mmol) in DMF (20 mL) were added with HATU (2.2 g, 6 mmol) and DIEA (1 g, 8 mmol). The reaction mixture was stirred at RT for 2 hrs, then added with water (15 mL), extracted with DCM (20 mL) and washed with brine (20 mL). The organic layer was dried, concentrated and purified by CombiFlash (DCM:MeOH=7%) to get the product (1 g, 63%). MS: M/e 438 (M+1)⁺

### Step B: N-(3-((1r, 3r)-3-cyano-1-((4-methyl-4H-1,2,4-triazol-3-yl)methyl) cyclobutyl)phenyl)-7-formyl-1H-pyrrolo[3,2-b]pyridine-5-carboxamide

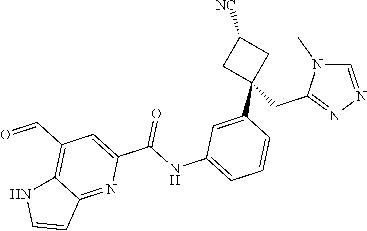 [SMILES: Cn1cnnc1C[C@]1(c2cccc(NC(=O)c3cc(C=O)c4[nH]ccc4n3)c2)C[C@H](C#N)C1]

To a solution of N-(3-((1r, 3r)-3-cyano-1-((4-methyl-4H-1,2,4-triazol-3-yl) methyl)cyclobutyl)phenyl)-7-vinyl-1H-pyrrolo[3,2-b]pyridine-5-carboxamide (1 g, 2.3 mmol) and K₂Os₄·2H₂O (62 mg, 0.23 mmol) in dioxane (20 mL) and water (4 mL) was cooled under ice bath. NaIO₄ (1.5 g, 6.9 mmol) was added in portionwise. Then it was stirred at RT for 3 hrs. The thickness suspension was filtered and washed with methanol (5 mL). The filtrate was concentrated to get the residue, which was added with water (10 mL) and extracted with DCM (15 mL). The crude product was purified by CombiFlash (DCM:MeOH=7%) to get the pure product (750 mg, 75%). MS: M/e 440 (M+1)⁺

### Step C: N-(3-((1r, 3r)-3-cyano-1-((4-methyl-4H-1,2,4-triazol-3-yl)methyl)cyclo butyl)phenyl)-7-((((1-methylcyclopentyl)methyl)amino)methyl)-1H-pyrrolo[3,2-b]pyridine-5-carboxamide

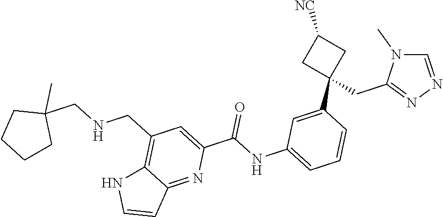 [SMILES: Cn1cnnc1C[C@]1(c2cccc(NC(=O)c3cc(CNCC4(C)CCCC4)c4[nH]ccc4n3)c2)C[C@H](C#N)C1]

A solution of N-(3-((1r, 3r)-3-cyano-1-((4-methyl-4H-1,2,4-triazol-3-yl)methyl) cyclobutyl)phenyl)-7-formyl-1H-pyrrolo[3,2-b]pyridine-5-carboxamide (50 mg, 0.11 mmol), (1-methylcyclopentyl)methanamine hydrochloride (33 mg, 0.22 mmol) and TEA (22 mg, 0.22 mmol) in DCM (2 mL) was stirred at RT for 10 mins. Then it was cooled under ice bath, and NaBH(OAc)₃ (47 mg, 0.22 mmol) was added. The reaction mixture was stirred at RT for 6 hrs, more of NaBH(OAc)₃ (47 mg, 0.22 mmol) was added and it was stirred overnight. The reaction mixture was added with water (5 mL), basified with NaHCO₃ solution to pH=7-8 and extracted with DCM (10 mL). The organic layer was dried, concentrated and purified by Prep-TLC (DCM:MeOH=8:1) to get the product (25 mg, 41%). ¹H NMR (400 MHz, CD₃OD) δ 8.17 (s, 2H), 7.82 (s, 1H), 7.73 (d, J=8.0 Hz, 1H), 7.53 (s, 1H), 7.33 (t, J=4.0 Hz, 1H), 6.84 (s, 1H), 6.81 (d, J=8.0 Hz, 1H), 4.55 (s, 2H), 3.41 (s, 2H), 3.18-3.15 (m, 1H), 3.03-2.97 (m, 4H), 2.91 (s, 2H), 2.82 (s, 3H), 1.70-1.66 (m, 4H), 1.49 (s, 4H), 1.10 (s, 3H). ppm. MS: M/e 537 (M+1)⁺

### Compound B24: 3-chloro-N-(3-((1s, 3s)-3-(cyanomethyl)-1-(4-methyl-4H-1,2,4-triazol-3-yl)cyclobutyl)phenyl)-7-(((1-methylcyclopropyl)amino)methyl)-1H-pyrrolo[3,2-b]pyridine-5-carboxamide

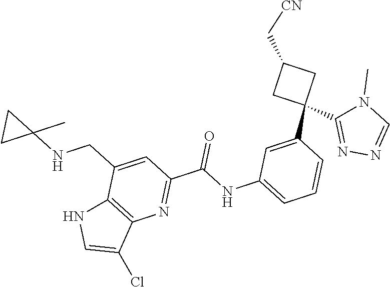 [SMILES: Cn1cnnc1[C@]1(c2cccc(NC(=O)c3cc(CNC4(C)CC4)c4[nH]cc(Cl)c4n3)c2)C[C@H](CC#N)C1]

### Step A: 3-chloro-7-(((1-methylcyclopropyl)amino)methyl)-1H-pyrrolo[3,2-b]pyridine-5-carbonitrile

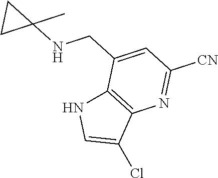 [SMILES: CC1(NCc2cc(C#N)nc3c(Cl)c[nH]c23)CC1]

To a solution of 3-chloro-7-formyl-1H-pyrrolo[3,2-b]pyridine-5-carbonitrile (65 mg, 0.317 mmol), 1-methylcyclopropan-1-amine hydrochloride (68 mg, 0.634 mmol) and Et₃N (64 mg, 0.634 mmol) in DCM (2 ml) was stirred at room temperature for 1 hour, then added NaBH(OAc)₃ (269 mg, 1.268 mmol). The mixture solution was stirred at 25° C. for 36 hours. The reaction mixture was concentrated under reduced pressure to give the crude product. The crude product was purified by flash column chromatography (DCM:MeOH=20:1) to give the titled compound (80 mg, 92%). MS: M/e 261 (M+1)⁺

### Step B: 3-chloro-7-(((1-methylcyclopropyl)amino)methyl)-1H-pyrrolo[3,2-b]pyridine-5-carboxylic acid

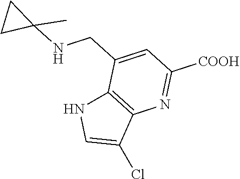 [SMILES: CC1(NCc2cc(C(=O)O)nc3c(Cl)c[nH]c23)CC1]

To a solution of 3-chloro-7-(((1-methylcyclopropyl)amino)methyl)-1H-pyrrolo[3,2-b]pyridine-5-carbonitrile (80 mg, 0.308 mmol) in H₂SO₄ (1 mL, 6N) was stirred at 100° C. for 16 hours. After completed, added NH₃/MeOH (6N) to adjust PH-8. After filtration, the solution was concentrated under vacuum and free-dried to give the crude product (80 mg, crude). MS: M/e 280 (M+1)⁺

### Step C: 3-chloro-N-(3-((1s, 3s)-3-(cyanomethyl)-1-(4-methyl-4H-1,2,4-triazol-3-yl)cyclobutyl)phenyl)-7-(((1-methylcyclopropyl)amino)methyl)-1H-pyrrolo[3,2-b]pyridine-5-carboxamide

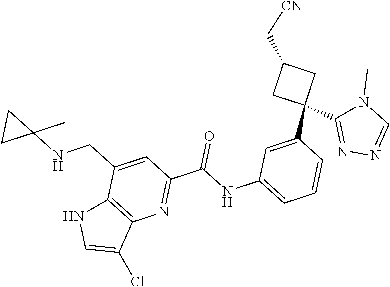 [SMILES: Cn1cnnc1[C@]1(c2cccc(NC(=O)c3cc(CNC4(C)CC4)c4[nH]cc(Cl)c4n3)c2)C[C@H](CC#N)C1]

To a solution of 3-chloro-7-(((1-methylcyclopropyl)amino)methyl)-1H-pyrrolo[3,2-b]pyridine-5-carboxylic acid (40 mg, 0.143 mmol), 2-((1s, 3s)-3-(3-aminophenyl)-3-(4-methyl-4H-1,2,4-triazol-3-yl)cyclobutyl)acetonitrile (38 mg, 0.143 mmol) in pyridine (1 mL) was added T₃P (274 mg, 0.430 mmol, 50% in EA). The mixture solution was stirred for 2 h at 25° C. After completed, the reaction was quenched with aq NH₄Cl (2 mL) and extracted with EA (2×10 mL), dried over Na₂SO₄ and concentrated under vacuum. The crude product was purified by Prep-HPLC to give the title compound (27 mg, 36%). MS: M/e 529 (M+1). ¹H NMR (400 MHz, DMSO) δ 10.40 (s, 1H), 8.31 (s, 1H), 8.05 (s, 1H), 7.97 (s, 1H), 7.89 (d, J=7.9 Hz, 2H), 7.37 (t, J=7.6 Hz, 1H), 7.02 (d, J=7.6 Hz, 1H), 4.12 (s, 2H), 3.20 (s, 3H), 2.86 (d, J=5.5 Hz, 2H), 2.80-2.65 (m, 5H), 1.27 (s, 3H), 0.57 (s, 2H), 0.32 (s, 2H) ppm.

### Compound B25: N-(3-((1s,3R)-3-(cyanomethyl)-1-(4-methyl-4H-1,2,4-triazol-3-yl)cyclobutyl)phenyl)-7-((((S)-1-cyclopentylethyl)amino)methyl)-1H-pyrrolo[3,2-b]pyridine-5-carboxamide

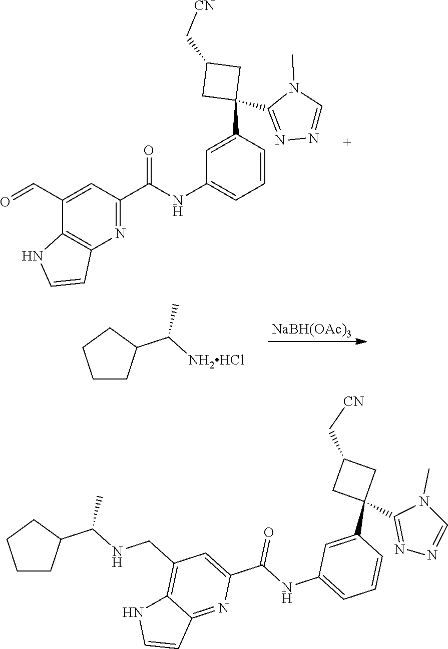

The compound B25 was synthesized starting from the corresponding starting materials according the similar procedures described as those of Compound B22 to get the product (95 mg, 65%). ¹H NMR (400 MHz, CD₃OD) δ 8.33 (s, 1H), 8.17 (s, 1H), 7.93 (s, 1H), 7.83 (s, 1H), 7.79 (d, J=8.0 Hz, 1H), 7.44 (t, J=8.0 Hz, 1H), 7.16 (d, J=8.0 Hz, 1H), 6.84 (s, 1H), 4.68-4.58 (m, 2H), 3.33 (s, 3H), 3.29-3.27 (m, 1H), 3.02 (d, J=8.0 Hz, 2H), 2.94-2.85 (m, 3H), 2.66 (d, J=4.0 Hz, 2H), 2.25-2.21 (m, 1H), 1.96-1.94 (m, 1H), 1.83 (s, 1H), 1.73-1.65 (m, 4H), 1.41 (d, J=4.0 Hz, 3H), 1.35 (s, 2H). ppm. MS: M/e 537 (M+1)⁺

### Compound B26: N-(3-((1s, 3s)-3-(cyanomethyl)-1-(4-methyl-4H-1,2,4-triazol-3-yl)cyclobutyl)phenyl)-7-(((1-methylcyclopropyl)amino)methyl)-1H-pyrrolo[3,2-b]pyridine-5-carboxamide

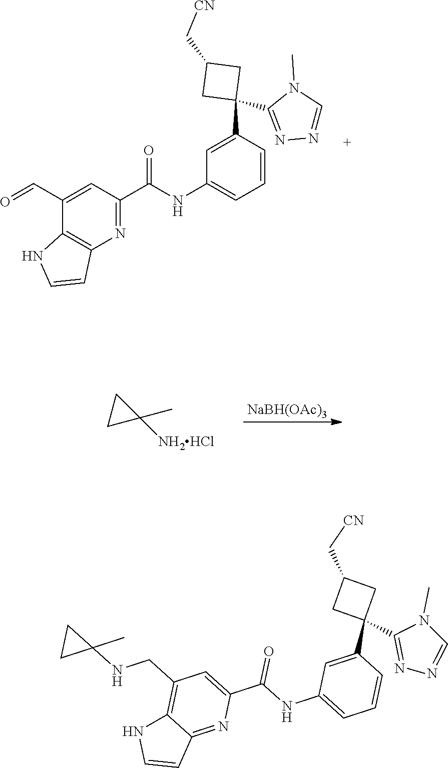

A solution of N-(3-((1s, 3s)-3-(cyanomethyl)-1-(4-methyl-4H-1,2,4-triazol-3-yl)cyclobutyl)phenyl)-7-formyl-1H-pyrrolo[3,2-b]pyridine-5-carboxamide (50 mg, 0.11 mmol), 1-methylcyclopropan-1-amine hydrochloride (24 mg, 0.22 mmol) and TEA (22 mg, 0.22 mmol) in DCM (2 mL) was stirred at r.t for 30 mins. Then it was cooled under ice bath, and NaBH(OAc)₃ (47 mg, 0.22 mmol) was added. The reaction mixture was stirred at r.t overnight. More of NaBH(OAc)₃ (47 mg, 0.22 mmol) was added and it continued to stir at r.t overnight. The reaction mixture was added with water (5 mL), basified with NaHCO₃ solution to pH=7-8 and extracted with DCM (10 mL). The organic layer was dried, concentrated and purified by prep.TLC (DCM:MeOH=9:1) to get the product (18 mg, 32%). ¹H NMR (400 MHz, CD3OD) δ 8.33 (s, 1H), 8.11 (s, 1H), 7.93 (s, 1H), 7.81 (s, 1H), 7.79 (d, J=8.0 Hz, 1H), 7.44 (t, J=8.0 Hz, 1H), 7.16 (d, J=8.0 Hz, 1H), 6.82 (d, J=4.0 Hz, 1H), 4.59 (s, 2H), 3.33 (s, 3H), 3.05-3.01 (m, 2H), 2.96-2.83 (m, 3H), 2.66 (d, J=4.0 Hz, 2H), 1.58 (s, 3H), 1.03 (s, 2H), 0.78-0.75 (m, 2H). ppm. MS: M/e 495 (M+1)⁺

### Compound B27: N-(3-((1s, 3s)-3-(cyanomethyl)-1-(4-methyl-4H-1,2,4-triazol-3-yl)cyclobutyl)phenyl)-7-(((1-methylcyclobutyl)amino)methyl)-1H-pyrrolo[3,2-b]pyridine-5-carboxamide

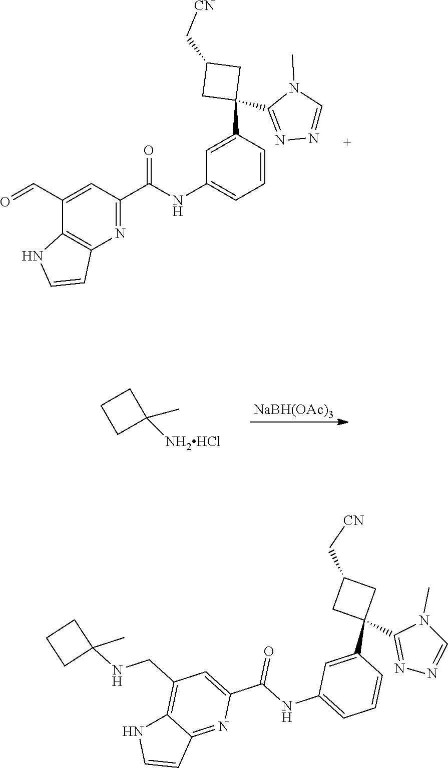

A solution of N-(3-((1s, 3s)-3-(cyanomethyl)-1-(4-methyl-4H-1,2,4-triazol-3-yl) cyclobutyl)phenyl)-7-formyl-1H-pyrrolo[3,2-b]pyridine-5-carboxamide (20 mg, 0.05 mmol), 1-methylcyclobutan-1-amine hydrochloride (12 mg, 0.1 mmol) and TEA (10 mg, 0.1 mmol) in DCM (2 mL) was stirred at r.t for 30 mins. Then it was cooled under ice bath, and NaBH(OAc)₃ (21 mg, 0.1 mmol) was added. The reaction mixture was stirred at r.t overnight. More of NaBH(OAc)₃ (21 mg, 0.1 mmol) was added and it continued to stir at r.t overnight. The reaction mixture was added with water (3 mL), basified with NaHCO₃ solution to pH=7-8 and extracted with DCM (8 mL). The organic layer was dried, concentrated and purified by prep.HPLC (Mobile Phase A: 0.1% FA-H2O, Mobile Phase B: 0.1% FA-ACN) to get the product (11 mg, 48%). ¹H NMR (400 MHz, CD3OD) δ 8.33 (s, 1H), 8.14 (s, 1H), 7.93 (s, 1H), 7.81 (s, 1H), 7.79 (d, J=4.0 Hz, 1H), 7.44 (t, J=8.0 Hz, 1H), 7.16 (d, J=8.0 Hz, 1H), 6.84 (d, J=4.0 Hz, 1H), 4.40 (s, 2H), 3.33 (s, 3H), 3.06-3.01 (m, 2H), 2.96-2.83 (m, 3H), 2.66 (d, J=4.0 Hz, 2H), 2.43-2.35 (m, 2H), 2.09-2.03 (m, 2H), 1.98-1.92 (m, 2H), 1.62 (s, 3H). ppm. MS: M/e 509 (M+1)⁺

### Compound B28: N-(3-((1s,3R)-3-(cyanomethyl)-1-(4-methyl-4H-1,2,4-triazol-3-yl)cyclobutyl)phenyl)-7-((((S)-1-cyclopropylethyl)amino)methyl)-1H-pyrrolo[3,2-b]pyridine-5-carboxamide

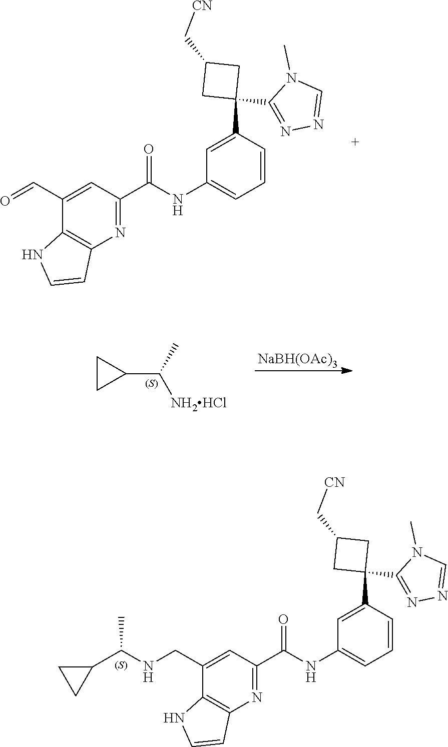

The compound B137 was synthesized starting from the corresponding starting materials according the similar procedures described as those of Compound B22 to get the product (28 mg, 30%). ¹H NMR (400 MHz, CD3OD) δ 8.33 (s, 1H), 8.16 (s, 1H), 7.93 (s, 1H), 7.82 (d, J=4.0 Hz, 1H), 7.79 (d, J=8.0 Hz, 1H), 7.44 (t, J=8.0 Hz, 1H), 7.15 (d, J=4.0 Hz, 1H), 6.84 (d, J=4.0 Hz, 1H), 4.71-4.58 (m, 2H), 3.33 (s, 3H), 3.05-3.01 (m, 2H), 2.97-2.83 (m, 3H), 2.66 (d, J=4.0 Hz, 3H), 1.47 (d, J=4.0 Hz, 3H), 1.07-1.03 (m, 1H), 0.84-0.80 (m, 1H), 0.71-0.67 (m, 1H), 0.58-0.54 (m, 1H), 0.36-0.30 (m, 1H) ppm. MS: M/e 509 (M+1)⁺

### Compound B29: N-(3-((1s, 3s)-3-(cyanomethyl)-1-(4-methyl-4H-1,2,4-triazol-3-yl)cyclobutyl)phenyl)-7-((isobutylamino)methyl)-1H-pyrrolo[3,2-b]pyridine-5-carboxamide

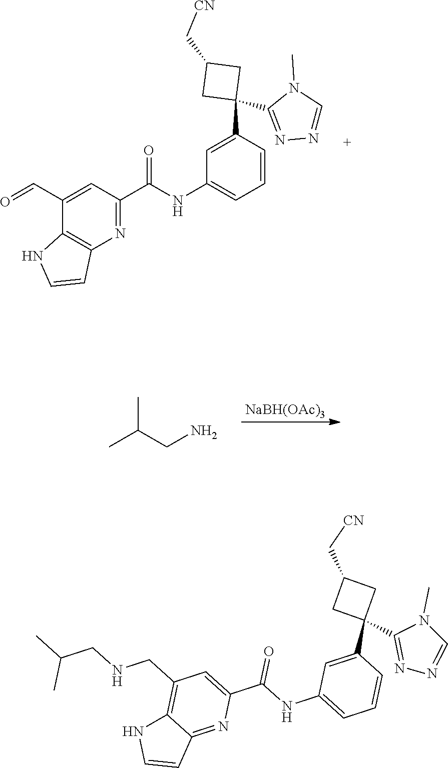

The compound B140 was synthesized starting from the corresponding starting materials according the similar procedures described as those of Compound B22 to get the product (29 mg, 42%). ¹H NMR (400 MHz, CD3OD) δ 8.33 (s, 1H), 8.14 (s, 1H), 7.94 (s, 1H), 7.81 (d, J=4.0 Hz, 1H), 7.79 (d, J=8.0 Hz, 1H), 7.44 (t, J=8.0 Hz, 1H), 7.16 (d, J=8.0 Hz, 1H), 6.83 (d, J=4.0 Hz, 1H), 4.53 (s, 2H), 3.33 (s, 3H), 3.05-3.01 (m, 2H), 2.95-2.83 (m, 5H), 2.66 (d, J=4.0 Hz, 2H), 2.07-2.01 (m, 1H), 1.03 (d, J=4.0 Hz, 6H) ppm. MS: M/e 497 (M+1)⁺

### Compound B30: N-(3-((1s, 3s)-3-(cyanomethyl)-1-(4-methyl-4H-1,2,4-triazol-3-yl)cyclobutyl)phenyl)-3-methyl-7-(((1-methylcyclopropyl)amino)methyl)-1H-pyrrolo[3,2-b]pyridine-5-carboxamide

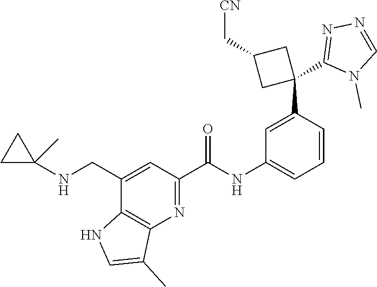 [SMILES: Cc1c[nH]c2c(CNC3(C)CC3)cc(C(=O)Nc3cccc([C@]4(c5nncn5C)C[C@@H](CC#N)C4)c3)nc12]

### Step A: 7-chloro-3-iodo-1H-pyrrolo[3,2-b]pyridine-5-carbonitrile

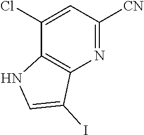 [SMILES: N#Cc1cc(Cl)c2[nH]cc(I)c2n1]

To a solution of 7-chloro-1H-pyrrolo[3,2-b]pyridine-5-carbonitrile (1 g, 5.6 mmol) in ACN (20 mL) was added NIS (1.4 g, 6.1 mmol). The reaction mixture was stirred for 0.5 h at 0° C. After completed, the solvent was removed by in vacuo. The residue was diluted in water and the mixture was filtrated, the solid was washed with water (3×20 mL), dried to afford the title compound (1.8 g, impure). ¹H NMR (400 MHz, DMSO-d₆) δ 12.97 (s, 1H), 8.23 (s, 1H), 8.10 (d, J=1.6 Hz, 1H). MS: M/e 303.9, 305.9 (M+H)⁺.

### Step B: 7-chloro-3-iodo-1-((2-(trimethylsilyl)ethoxy)methyl)-1H-pyrrolo[3,2-b]pyridine-5-carbonitrile

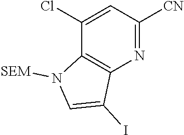 [SMILES: C[Si](C)(C)CCOCn1cc(I)c2nc(C#N)cc(Cl)c21]

To a solution of 7-chloro-3-iodo-1H-pyrrolo[3,2-b]pyridine-5-carbonitrile (1.8 g, impure) in THF (30 mL) was added NaH (270 mg, 6.75 mmol). After stirring for 10 min, SEMCl (1.4 g, 8.38 mmol) was added dropwise. The reaction mixture was stirred for 12 h at 25° C. After completed, the reaction was quenched with aq NH₄Cl (20 mL) and extracted with DCM (3×50 mL), dried over Na₂SO₄ and concentrated under vacuum. The residue was purified by column chromatography on silica gel eluting with ethyl acetate in petroleum ether (15%) to afford the title compound (2.4 g, 99% for two steps). ¹H NMR (400 MHz, CD₃OD) δ 8.13 (s, 1H), 7.85 (d, J=1.6 Hz, 1H), 5.83 (s, 2H), 3.57 (t, J=7.8 Hz, 2H), 0.86 (t, J=7.8 Hz, 2H), −0.08 (s, 9H). MS: M/e 434, 436 (M+H)⁺.

### Step C: 7-chloro-3-methyl-1-((2-(trimethylsilyl)ethoxy)methyl)-1H-pyrrolo[3,2-b]pyridine-5-carbonitrile

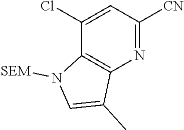 [SMILES: Cc1cn(COCC[Si](C)(C)C)c2c(Cl)cc(C#N)nc12]

To a solution of 7-chloro-3-iodo-1-((2-(trimethylsilyl)ethoxy)methyl)-1H-pyrrolo[3,2-b]pyridine-5-carbonitrile (2.3 g, 5.29 mmol), tetramethyltin (1.9 g, 10.61 mmol) in DMF (10 mL) was added LiCl (670 mg, 15.95 mmol) and PdCl₂(PPh₃)₂(370 mg, 0.52 mmol) under N₂. The reaction mixture was stirred for 12 h at 105° C. After completed, the reaction was quenched with aq NH₄Cl (20 mL) and extracted with DCM (3×50 mL), dried over Na₂SO₄ and concentrated under vacuum. The residue was purified by column chromatography on silica gel eluting with ethyl acetate in petroleum ether (20%) to afford the title compound (1.2 g, 71%). ¹H NMR (400 MHz, CD₃OD) δ 7.75-7.69 (m, 2H), 5.79 (s, 2H), 3.55 (t, J=7.8 Hz, 2H), 2.35 (s, 3H), 0.85 (t, J=7.8 Hz, 2H), −0.08 (s, 9H). MS: M/e 322, 324. (M+H)⁺.

### Step D: 7-chloro-3-methyl-1-((2-(trimethylsilyl)ethoxy)methyl)-1H-pyrrolo[3,2-b]pyridine-5-carboxylic acid

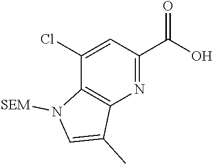 [SMILES: Cc1cn(COCC[Si](C)(C)C)c2c(Cl)cc(C(=O)O)nc12]

To a solution of 7-chloro-3-methyl-1-((2-(trimethylsilyl)ethoxy)methyl)-1H-pyrrolo[3,2-b]pyridine-5-carbonitrile (1.1 g, 3.4 mmol) in EtOH (20 mL) was added NaOH (3.5 mL, 35 mmol, 10 N). The reaction mixture was stirred for 12 h at 110° C. in seal tube. The reaction mixture was stirred for 12 h at 110° C. After completed, the solvent was concentrated under vacuum. The residue was diluted with water and neutralized with 2N HCl and extracted with DCM/MeOH (10/1, 3×100 mL), dried over Na₂SO₄ and concentrated under vacuum. The crude product (1.1 g) was obtained as a yellow solid. MS: M/e 341, 343. (M+H)⁺.

### Step E: 7-chloro-N-(3-((1s, 3s)-3-(cyanomethyl)-1-(4-methyl-4H-1,2,4-triazol-3-yl)cyclobutyl)phenyl)-3-methyl-1-((2-(trimethylsilyl)ethoxy)methyl)-1H-pyrrolo[3,2-b]pyridine-5-carboxamide

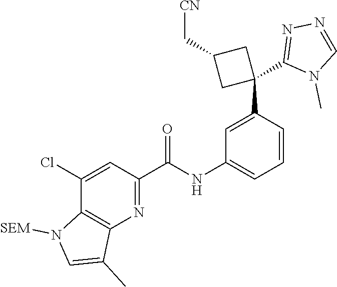 [SMILES: Cc1cn(COCC[Si](C)(C)C)c2c(Cl)cc(C(=O)Nc3cccc([C@]4(c5nncn5C)C[C@@H](CC#N)C4)c3)nc12]

To a solution of 7-chloro-3-methyl-1-((2-(trimethylsilyl)ethoxy)methyl)-1H-pyrrolo[3,2-b]pyridine-5-carboxylic acid (1.1 g, impure), 2-((1s, 3s)-3-(3-aminophenyl)-3-(4-methyl-4H-1,2,4-triazol-3-yl)cyclobutyl)acetonitrile (860 mg, 3.22 mmol), DIEA (830 mg, 6.43 mmol) in DMF (10 mL) was added HATU (1.8 g, 4.73 mmol). The reaction mixture was stirred for 0.5 h at 25° C. After completed, the reaction was quenched with aq NH₄Cl (20 mL) and extracted with DCM (3×50 mL), dried over Na₂SO₄ and concentrated under vacuum. The residue was purified by column chromatography on silica gel eluting with methanol in dichloromethane (10%) to afford the title compound (1.9 g, impure). MS: M/e 590, 592 (M+H)⁺.

### Step F: N-(3-((1s, 3s)-3-(cyanomethyl)-1-(4-methyl-4H-1,2,4-triazol-3-yl)cyclobutyl)phenyl)-3-methyl-1-((2-(trimethylsilyl)ethoxy)methyl)-7-vinyl-1H-pyrrolo[3,2-b]pyridine-5-carboxamide

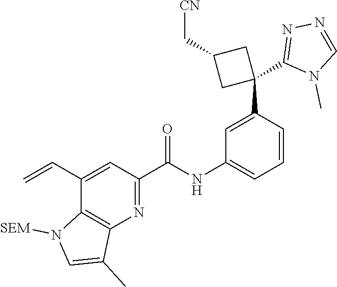 [SMILES: C=Cc1cc(C(=O)Nc2cccc([C@]3(c4nncn4C)C[C@@H](CC#N)C3)c2)nc2c(C)cn(COCC[Si](C)(C)C)c12]

To a solution of 7-chloro-N-(3-((1s, 3s)-3-(cyanomethyl)-1-(4-methyl-4H-1,2,4-triazol-3-yl)cyclobutyl)phenyl)-3-methyl-1-((2-(trimethylsilyl)ethoxy)methyl)-1H-pyrrolo[3,2-b]pyridine-5-carboxamide (1.8 g, impure), potassium trifluoro(vinyl)borate (530 mg, 3.96 mmol) in Dioxane (20 mL)/H₂O (2 mL) was added Xphos G2 Pd (120 mg, 0.152 mmol) and Cs₂CO₃ (2 g, 6.13 mmol) under N₂. The reaction mixture was stirred for 8 h at 80° C. After completed, the mixture was filtered and the filate was collected and concentrated under vacuum. The residue was purified by column chromatography on silica gel eluting with methanol in dichloromethane (10%) to afford the title compound (1.7 g, 96%). ¹H NMR (400 MHz, CD₃OD) δ 8.33 (s, 1H), 8.13 (s, 1H), 7.97 (s, 1H), 7.79 (d, J=7.9 Hz, 1H), 7.67-7.56 (m, 1H), 7.53 (s, 1H), 7.44 (t, J=8.0 Hz, 1H), 7.14 (d, J=7.8 Hz, 1H), 6.04 (d, J=17.2 Hz, 1H), 5.63 (d, J=11.2 Hz, 1H), 5.56 (s, 2H), 3.57 (t, J=7.8 Hz, 2H), 3.34 (s, 3H), 3.09-3.01 (m, 2H), 3.00-2.86 (m, 3H), 2.67 (d, J=6.0 Hz, 2H), 2.44 (s, 3H), 0.88 (t, J=7.8 Hz, 2H), −0.07 (s, 9H). MS: M/e 582. (M+H)⁺.

### Step G: N-(3-((1s, 3s)-3-(cyanomethyl)-1-(4-methyl-4H-1,2,4-triazol-3-yl)cyclobutyl)phenyl)-3-methyl-7-vinyl-1H-pyrrolo[3,2-b]pyridine-5-carboxamide

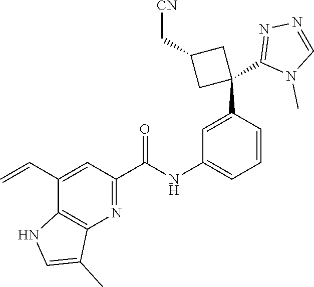 [SMILES: C=Cc1cc(C(=O)Nc2cccc([C@]3(c4nncn4C)C[C@@H](CC#N)C3)c2)nc2c(C)c[nH]c12]

To a solution of N-(3-((1s, 3s)-3-(cyanomethyl)-1-(4-methyl-4H-1,2,4-triazol-3-yl)cyclobutyl)phenyl)-3-methyl-1-((2-(trimethylsilyl)ethoxy)methyl)-7-vinyl-1H-pyrrolo[3,2-b]pyridine-5-carboxamide (1.5 g, 2.57 mmol) in DCM (2 mL) was added TFA (18 mL). The reaction mixture was stirred for 12 h at 25° C. After completed, the solvent was removed by in vacuo. The residue was dissolved in MeOH (20 mL) and Ethylenediamine (4 mL) was added. The reaction mixture was stirred for 12 h at 25° C. After completed, the solvent was removed by in vacuo. The residue was purified by column chromatography on silica gel eluting with methanol in dichloromethane (10%) to afford the title compound (1.2 g, crude). MS: M/e 452. (M+H)⁺.

### Step H: N-(3-((1s, 3s)-3-(cyanomethyl)-1-(4-methyl-4H-1,2,4-triazol-3-yl)cyclobutyl)phenyl)-7-formyl-3-methyl-1H-pyrrolo[3,2-b]pyridine-5-carboxamide

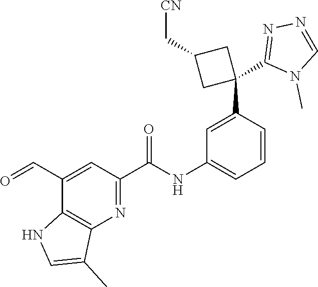 [SMILES: Cc1c[nH]c2c(C=O)cc(C(=O)Nc3cccc([C@]4(c5nncn5C)C[C@@H](CC#N)C4)c3)nc12]

To a solution of N-(3-((1s, 3s)-3-(cyanomethyl)-1-(4-methyl-4H-1,2,4-triazol-3-yl)cyclobutyl)phenyl)-3-methyl-7-vinyl-1H-pyrrolo[3,2-b]pyridine-5-carboxamide (1.2 g, crude) and K₂OsO₄H2O (100 mg, 0.272 mmol) in dioxane (30 mL)/H₂O (6 mL) was added NaIO₄ (1.1 g, 5.14 mmol) at 0° C. The reaction mixture was stirred for 3 h at 25° C. After completed, the mixture was filtrated, the filate was collected and concentrated under vacuum. The residue was purified by column chromatography on silica gel eluting with methanol in dichloromethane (6%) to afford the title compound (1.5 g, crude). MS: M/e 454 (M+H)⁺.

### Step I: N-(3-((1s, 3s)-3-(cyanomethyl)-1-(4-methyl-4H-1,2,4-triazol-3-yl)cyclobutyl)phenyl)-3-methyl-7-(((1-methylcyclopropyl)amino)methyl)-1H-pyrrolo[3,2-b]pyridine-5-carboxamide

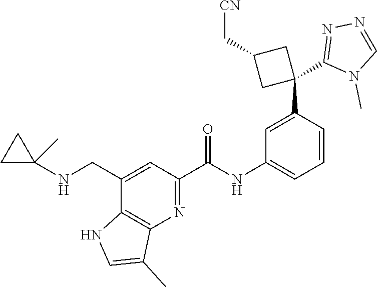 [SMILES: Cc1c[nH]c2c(CNC3(C)CC3)cc(C(=O)Nc3cccc([C@]4(c5nncn5C)C[C@@H](CC#N)C4)c3)nc12]

To a solution of N-(3-((1s, 3s)-3-(cyanomethyl)-1-(4-methyl-4H-1,2,4-triazol-3-yl)cyclobutyl)phenyl)-7-formyl-3-methyl-1H-pyrrolo[3,2-b]pyridine-5-carboxamide (50 mg, impure) and 1-methylcyclopropan-1-amine hydrochloride (18 mg, 0.167 mmol) in DCM (5 mL) was added NaBH(OAc)₃ (50 mg, 0.235 mmol). The reaction mixture was stirred for 12 h at 25° C. After completed, the reaction was quenched with aq NaHCO₃ (10 mL) and extracted with DCM (3×30 mL), dried over Na₂SO₄ and concentrated under vacuum. The residue was purified by prep-HPLC to afford the title compound (10 mg). ¹H NMR (500 MHz, CD₃OD) δ 8.32 (s, 1H), 8.02 (s, 1H), 7.96 (s, 1H), 7.77 (dd, J=8.2, 1.0 Hz, 1H), 7.46-7.40 (m, 2H), 7.12 (d, J=7.8 Hz, 1H), 4.20 (s, 2H), 3.34 (s, 3H), 3.08-3.01 (m, 2H), 3.01-2.90 (m, 1H), 2.89-2.81 (m, 2H), 2.65 (d, J=5.0 Hz, 2H), 2.45 (s, 3H), 1.37 (s, 3H), 0.66 (t, J=4.5 Hz, 2H), 0.41 (q, J=4.5 Hz, 2H). MS: M/e 509 (M+H)⁺.

### Compound B31: N-(3-((1s,3R)-3-(cyanomethyl)-1-(4-methyl-4H-1,2,4-triazol-3-yl)cyclobutyl)phenyl)-7-((((S)-1-cyclobutylethyl)amino)methyl)-1H-pyrrolo[3,2-b]pyridine-5-carboxamide

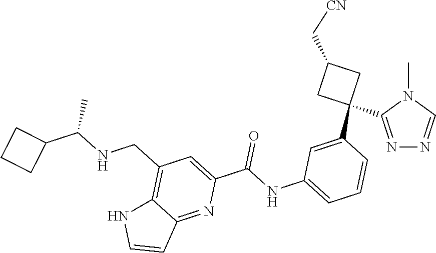 [SMILES: C[C@H](NCc1cc(C(=O)Nc2cccc([C@]3(c4nncn4C)C[C@@H](CC#N)C3)c2)nc2cc[nH]c12)C1CCC1]

A solution of N-(3-((1s, 3s)-3-(cyanomethyl)-1-(4-methyl-4H-1,2,4-triazol-3-yl) cyclobutyl)phenyl)-7-formyl-1H-pyrrolo[3,2-b]pyridine-5-carboxamide (100 mg, 0.23 mmol), (S)-1-cyclobutylethan-1-amine hydrochloride (62 mg, 0.46 mmol) and TEA (47 mg, 0.46 mmol) in DCM (5 mL) was stirred at r.t for 30 mins. Then it was cooled under ice bath, and NaBH(OAc)₃ (98 mg, 0.46 mmol) was added. The reaction mixture was stirred at r.t for 6 hrs, more of NaBH(OAc)₃ (49 mg, 0.23 mmol) was added and it was stirred overnight. The reaction mixture was added with water (6 mL), basified with NaHCO₃ solution to pH=7-8 and extracted with DCM (15 mL). The organic layer was dried, concentrated and purified by prep.TLC (DCM:MeOH=10:1) to get the product (65 mg, 55%). ¹H NMR (400 MHz, CD3OD) δ 8.33 (s, 1H), 8.15 (s, 1H), 7.92 (s, 1H), 7.83 (d, J=4.0 Hz, 1H), 7.79 (d, J=8.0 Hz, 1H), 7.45 (d, J=8.0 Hz, 1H), 7.18 (d, J=8.0 Hz, 1H), 6.86 (d, J=4.0 Hz, 1H), 4.60-4.51 (q, 2H), 3.33-3.10 (m, 4H), 3.04 (t, J=4.0 Hz, 2H), 2.97-2.91 (m, 1H), 2.85 (t, J=4.0 Hz, 2H), 2.66 (d, J=4.0 Hz, 2H), 2.65-2.60 (m, 1H), 2.24-2.20 (m, 1H), 2.14-2.09 (m, 1H), 1.99-1.93 (m, 2H), 1.92-1.83 (m, 2H), 1.31 (d, J=8.0 Hz, 3H). ppm. MS: M/e 523 (M+1)⁺

### Compound B32: 7-(((cyclobutylmethyl)amino)methyl)-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide

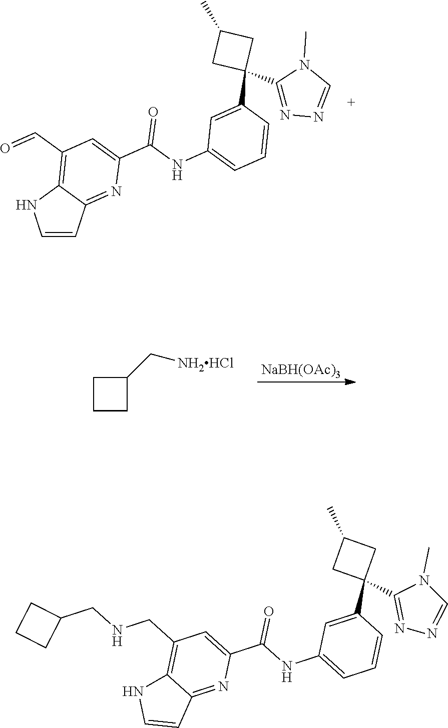

A solution of 7-formyl-N-(3-((1s, 3s)-3-methyl-1-(4-methyl-4H-1,2,4-triazol-3-yl) cyclobutyl)phenyl)-1H-pyrrolo[3,2-b]pyridine-5-carboxamide (30 mg, 0.07 mmol), cyclobutylmethanamine hydrochloride (17 mg, 0.14 mmol) and TEA (14 mg, 0.14 mmol) in DCM (1 mL) was stirred at r.t for 30 mins. Then it was cooled under ice bath, and NaBH(OAc)₃ (30 mg, 0.14 mmol) was added. The reaction mixture was stirred at r.t for 6 hrs, more of NaBH(OAc)₃ (15 mg, 0.07 mmol) was added and it was stirred overnight. The reaction mixture was added with water (3 mL), basified with NaHCO₃ solution to pH=7-8 and extracted with DCM (8 mL). The organic layer was dried, concentrated and purified by prep.TLC (DCM:MeOH=10:1) to get the product (15 mg, 43%). ¹H NMR (400 MHz, CD3OD) δ 8.31 (s, 1H), 8.14 (s, 1H), 7.90 (s, 1H), 7.83 (d, J=4.0 Hz, 1H), 7.75 (d, J=8.0 Hz, 1H), 7.41 (t, J=8.0 Hz, 1H), 7.15 (d, J=8.0 Hz, 1H), 6.85 (d, J=4.0 Hz, 1H), 4.57 (s, 2H), 3.31 (s, 3H), 3.18 (d, J=4.0 Hz, 2H), 2.97-2.93 (m, 2H), 2.77-2.66 (m, 2H), 2.61-2.57 (m, 2H), 2.23-2.17 (m, 2H), 2.05-1.98 (m, 1H), 1.93-1.83 (m, 3H), 1.16 (d, J=8.0 Hz, 3H). ppm. MS: M/e 484 (M+1)⁺

### Compound B33: N-(3-((1s, 3s)-3-methyl-1-(4-methyl-4H-1,2,4-triazol-3-yl)cyclobutyl)phenyl)-7-((((1s, 3s)-3-methylcyclobutyl)amino)methyl)-1H-pyrrolo[3,2-b]pyridine-5-carboxamide

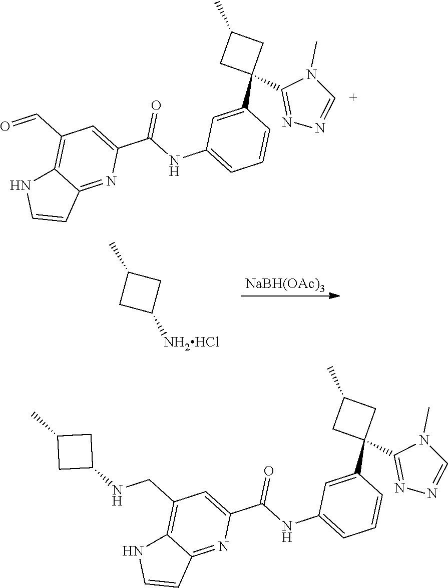

The compound B33 was synthesized starting from the corresponding starting materials according the similar procedures described as those of Compound B1 to get the product (13 mg, 37%). H NMR (400 MHz, CD3OD) δ 8.31 (s, 1H), 8.11 (s, 1H), 7.91 (s, 1H), 7.82 (d, J=4.0 Hz, 1H), 7.75 (d, J=8.0 Hz, 1H), 7.41 (t, J=8.0 Hz, 1H), 7.14 (d, J=8.0 Hz, 1H), 6.84 (d, J=4.0 Hz, 1H), 4.47 (s, 2H), 3.70-3.64 (m, 1H), 3.30 (s, 3H), 2.97-2.93 (m, 2H), 2.72-2.66 (m, 1H), 2.61-2.57 (m, 2H), 2.52-2.48 (m, 2H), 2.24-2.19 (m, 1H), 1.80-1.74 (m, 2H), 1.16 (d, J=4.0 Hz, 3H), 1.14 (d, J=8.0 Hz, 3H) ppm. MS: M/e 484 (M+1)⁺

### Compound B34:7-(((3R,4S)-4-fluoro-3-methylpiperidin-1-yl)methyl)-3-methyl-N-(3-((1s,3S)-3-methyl-1-(4-methyl-4H-1,2,4-triazol-3-yl)cyclobutyl)phenyl)-1H-pyrrolo[3,2-b]pyridine-5-carboxamide

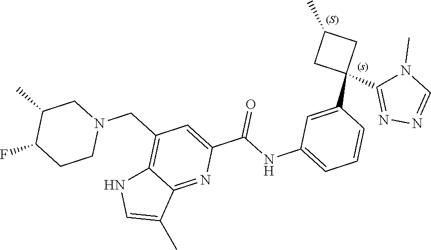

### Step A: 7-chloro-3-methyl-N-(3-((1s, 3s)-3-methyl-1-(4-methyl-4H-1,2,4-triazol-3-yl)cyclobutyl)phenyl)-1-((2-(trimethylsilyl)ethoxy)methyl)-1H-pyrrolo[3,2-b]pyridine-5-carboxamide

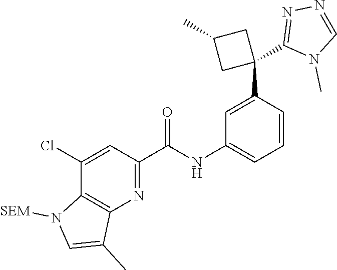 [SMILES: Cc1cn(COCC[Si](C)(C)C)c2c(Cl)cc(C(=O)Nc3cccc([C@]4(c5nncn5C)C[C@@H](C)C4)c3)nc12]

To a solution of 7-chloro-3-methyl-1-((2-(trimethylsilyl)ethoxy)methyl)-1H-pyrrolo[3,2-b]pyridine-5-carboxylic acid (510 mg, 1.5 mmol), 3-((1s, 3s)-3-methyl-1-(4-methyl-4H-1,2,4-triazol-3-yl)cyclobutyl)aniline (363 mg, 1.5 mmol), DIPEA (387 mg, 3.0 mmol) in DMF (35 mL) was added HATU (856 mg, 2.25 mmol). The reaction mixture was stirred for 6 h at 25° C. After completed, the reaction was quenched with aq NH₄Cl (10 mL) and extracted with EA (3×45 mL), dried over Na₂SO₄ and concentrated under vacuum. The residue was purified by flash column chromatography (DCM:MeOH=20:1) to give the titled compound (760 mg, 90%). MS: M/e 565 (M+1)⁺

### Step B: 3-methyl-N-(3-((1s, 3s)-3-methyl-1-(4-methyl-4H-1,2,4-triazol-3-yl)cyclobutyl)phenyl)-1-((2-(trimethylsilyl)ethoxy)methyl)-7-vinyl-1H-pyrrolo[3,2-b]pyridine-5-carboxamide

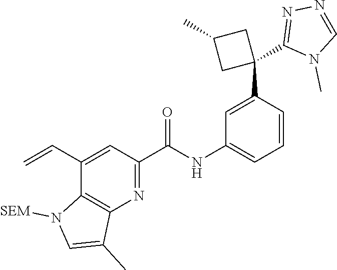 [SMILES: C=Cc1cc(C(=O)Nc2cccc([C@]3(c4nncn4C)C[C@@H](C)C3)c2)nc2c(C)cn(COCC[Si](C)(C)C)c12]

To a solution of 7-chloro-3-methyl-N-(3-((1s, 3s)-3-methyl-1-(4-methyl-4H-1,2,4-triazol-3-yl)cyclobutyl)phenyl)-1-((2-(trimethylsilyl)ethoxy)methyl)-1H-pyrrolo[3,2-b]pyridine-5-carboxamide (715 mg, 1.267 mmol), potassium trifluoro(vinyl)borate (221 mg, 1.647 mmol), XPhos G2 Pd (50 mg, 0.063 mmol) and Cs₂CO₃ (826 mg, 2.534 mmol) in dioxane (30 mL) and H₂O (3 mL) was stirred at 80° C. under N₂ for 12 hours. The reaction mixture was quenched with saturated NaCl aq. (20 mL), extracted with EA (50 mL×2), combined, washed brine (30 mL×2), dried and concentrated to dryness. The resulting residue was purified by flash column chromatography (DCM:MeOH=20:1) to give the titled compound (620 mg, 88%). MS: M/e 557 (M+1)⁺

### Step C: 3-methyl-N-(3-((1s, 3s)-3-methyl-1-(4-methyl-4H-1,2,4-triazol-3-yl)cyclobutyl)phenyl)-7-vinyl-1H-pyrrolo[3,2-b]pyridine-5-carboxamide

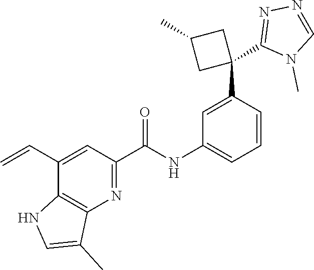 [SMILES: C=Cc1cc(C(=O)Nc2cccc([C@]3(c4nncn4C)C[C@@H](C)C3)c2)nc2c(C)c[nH]c12]

To a solution of 3-methyl-N-(3-((1s, 3s)-3-methyl-1-(4-methyl-4H-1,2,4-triazol-3-yl)cyclobutyl)phenyl)-1-((2-(trimethylsilyl)ethoxy)methyl)-7-vinyl-1H-pyrrolo[3,2-b]pyridine-5-carboxamide (620 mg, 1.115 mmol) in DCM (2 mL) was added TFA (10 mL). The reaction mixture was stirred at room temperature for 16 hours. After completed, the solvent was concentrated under vacuum to give the intermediate. The intermediate was stirred at NH₃H2O (5 mL) and MeOH (3 mL) at room temperature for 2 hours. Then the reaction solution was concentrated under vacuum to give the crude product, which was purified by flash column chromatography (DCM:MeOH=15:1) to give the titled compound (700 mg, crude). MS: M/e 427 (M+1)⁺

### Step D: 7-formyl-3-methyl-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide

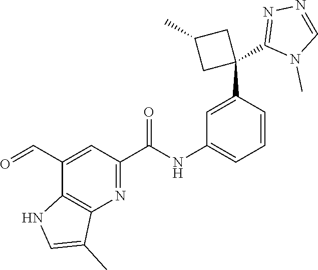 [SMILES: Cc1c[nH]c2c(C=O)cc(C(=O)Nc3cccc([C@]4(c5nncn5C)C[C@@H](C)C4)c3)nc12]

To a solution of 3-methyl-N-(3-((1s, 3s)-3-methyl-1-(4-methyl-4H-1,2,4-triazol-3-yl)cyclobutyl)phenyl)-7-vinyl-1H-pyrrolo[3,2-b]pyridine-5-carboxamide (475 mg, 1.115 mmol), K₂OsO₄ 2H₂O (41 mg, 0.1115 mmol) in dioxane (30 mL) and H₂O (5 mL) at 0° C. was added NaIO₄ (477 mg, 2.23 mmol). The mixture was stirred at room temperature for 4 hours. After filtration, the solution was concentrated to dryness. The resulting residue was purified by flash column chromatography (DCM:MeOH=15:1) to give the titled compound (260 mg, 55%). MS: M/e 429 (M+1)⁺

### Step E: 7-(((3R,4S)-4-fluoro-3-methylpiperidin-1-yl)methyl)-3-methyl-N-(3-((1s,3S)-3-methyl-1-(4-methyl-4H-1,2,4-triazol-3-yl)cyclobutyl)phenyl)-1H-pyrrolo[3,2-b]pyridine-5-carboxamide

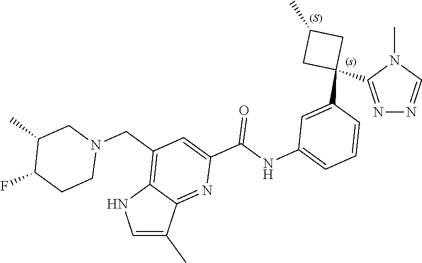

To a solution of 7-formyl-3-methyl-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide (20 mg, 0.047 mmol), (3R,4S)-4-fluoro-3-methylpiperidine hydrochloride (14 mg, 0.093 mmol) and Et₃N (9 mg, 0.093 mmol) in DCM (1 ml) was stirred at room temperature for 1 hour, then added NaBH(OAc)₃ (20 mg, 0.093 mmol). The mixture solution was stirred at 25° C. for 36 hours. The reaction mixture was concentrated under reduced pressure. The crude product was purified by Prep-HPLC to give the title compound (12 mg, 48%). MS: M/e 530 (M+1). ¹H NMR (400 MHz, DMSO) δ 11.19 (s, 1H), 10.46 (s, 1H), 8.30 (s, 1H), 7.93 (s, 1H), 7.89 (s, 1H), 7.81 (d, J=8.1 Hz, 1H), 7.56 (s, 1H), 7.36 (t, J=7.9 Hz, 1H), 7.01 (d, J=7.9 Hz, 1H), 4.66 (d, J=50.3 Hz, 1H), 3.85 (s, 2H), 3.20 (s, 3H), 2.85 (dd, J=9.4, 5.3 Hz, 2H), 2.64-2.53 (m, 5H), 2.43 (s, 3H), 2.28 (t, J=10.8 Hz, 1H), 2.12-2.03 (m, 1H), 2.00-1.77 (m, 3H), 1.09 (d, J=5.8 Hz, 3H), 0.88 (d, J=6.9 Hz, 3H) ppm.

### Compound B35: 7-((((S)-1-cyclobutylethyl)amino)methyl)-N-(3-((1s,3R)-3-methyl-1-(4-methyl-4H-1,2,4-triazol-3-yl)cyclobutyl)phenyl)-1H-pyrrolo[3,2-b]pyridine-5-carboxamide

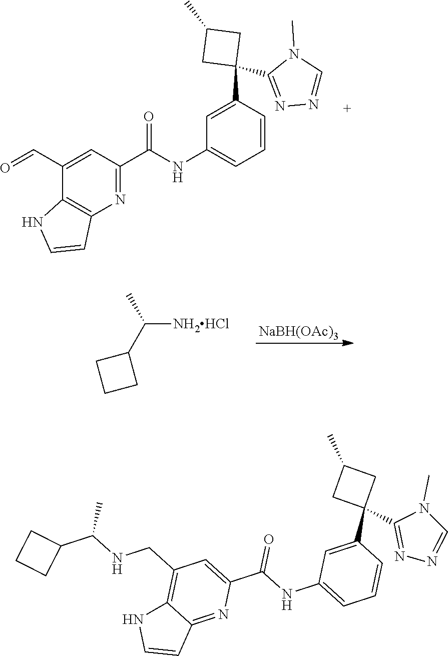

The compound B35 was synthesized starting from the corresponding starting materials according the similar procedures described as those of Compound B1 to get the product (18 mg, 50%). ¹H NMR (400 MHz, CD3OD) δ 8.30 (s, 1H), 8.07 (s, 1H), 7.94 (s, 1H), 7.76 (d, J=4.0 Hz, 1H), 7.75 (s, 1H), 7.41 (t, J=8.0 Hz, 1H), 7.11 (d, J=8.0 Hz, 1H), 6.83 (d, J=4.0 Hz, 1H), 4.38-4.26 (m, 2H), 3.33 (s, 3H), 2.95 (t, J=8.0 Hz, 2H), 2.84-2.81 (m, 1H), 2.73-3.68 (m, 1H), 2.59 (t, J=8.0 Hz, 2H), 2.43-2.38 (m, 1H), 2.15-2.11 (m, 1H), 2.03-2.00 (m, 1H), 1.94-1.87 (m, 1H), 1.85-1.76 (m, 3H), 1.16 (d, J=8.0 Hz, 3H), 1.12 (d, J=8.0 Hz, 3H) ppm. MS: M/e 498 (M+1)+

### Compound B36:7-(((cyclobutylmethyl)amino)methyl)-3-fluoro-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide

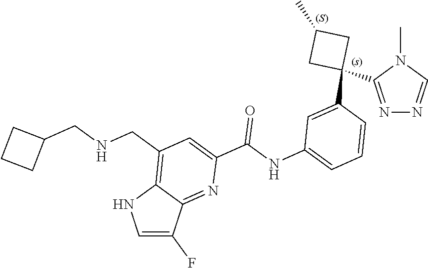

### Step A: 7-chloro-3-fluoro-1H-pyrrolo[3,2-b]pyridine-5-carbonitrile

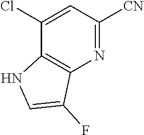 [SMILES: N#Cc1cc(Cl)c2[nH]cc(F)c2n1]

To a solution of 7-chloro-1H-pyrrolo[3,2-b]pyridine-5-carbonitrile (5.0 g, 28.25 mmol), NaHCO₃ (4.75 g, 56.5 mmol) in DMF (60 mL) was added select flour (15.0 g, 42.37 mmol). The reaction mixture was stirred for 12 hrs at 60° C. After completed, the reaction solution was quenched with aq NaCl (20 mL) and extracted with EA (3×60 mL), dried over Na₂SO₄ and concentrated under vacuum. The residue was purified by flash column chromatography (DCM: EA=20:1) to give the titled compound (1.2 g, 80% purity). MS: M/e 196 (M+1)⁺

### Step B: 7-chloro-3-fluoro-1H-pyrrolo[3,2-b]pyridine-5-carboxylic acid

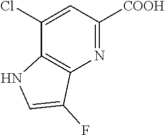 [SMILES: O=C(O)c1cc(Cl)c2[nH]cc(F)c2n1]

To a solution of 7-chloro-3-fluoro-1H-pyrrolo[3,2-b]pyridine-5-carbonitrile (300 mg, 1.538 mmol) in HCl (6N) was stirred at 100° C. for 16 hours. After completed, the reaction solution was concentrated under vacuum to give the crude product. Added 2N NaOH to adjust PH-7 and free-dried to give the crude product (300 mg, crude). MS: M/e 215 (M+1)⁺

### Step C: 7-chloro-3-fluoro-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide

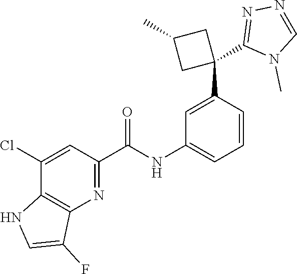 [SMILES: Cn1cnnc1[C@]1(c2cccc(NC(=O)c3cc(Cl)c4[nH]cc(F)c4n3)c2)C[C@H](C)C1]

To a solution of 7-chloro-3-fluoro-1H-pyrrolo[3,2-b]pyridine-5-carboxylic acid (280 mg, 1.308 mmol), 3-((1s, 3s)-3-methyl-1-(4-methyl-4H-1,2,4-triazol-3-yl)cyclobutyl)aniline (317 mg, 1.308 mmol) and EDCI (500 mg, 2.616 mmol) in DCM (35 mL) was stirred for 4 hrs at 25° C. After completed, the reaction was quenched with aq NH₄Cl (10 mL) and extracted with DCM (3×45 mL), dried over Na₂SO₄ and concentrated under vacuum. The residue was purified by flash column chromatography (DCM:MeOH=20:1) to give the titled compound (210 mg, 37%). MS: M/e 439 (M+1)⁺

### Step D: 3-fluoro-N-(3-((1s, 3s)-3-methyl-1-(4-methyl-4H-1,2,4-triazol-3-yl)cyclobutyl)phenyl)-7-vinyl-1H-pyrrolo[3,2-b]pyridine-5-carboxamide

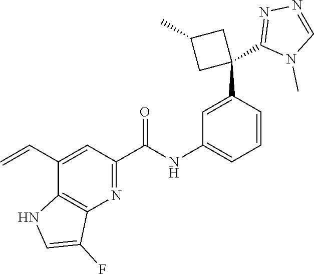 [SMILES: C=Cc1cc(C(=O)Nc2cccc([C@]3(c4nncn4C)C[C@@H](C)C3)c2)nc2c(F)c[nH]c12]

To a solution of 7-chloro-3-fluoro-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide (205 mg, 0.468 mmol), potassium trifluoro(vinyl)borate (82 mg, 0.608 mmol), XPhos G2 Pd (18 mg, 0.023 mmol) and Cs₂CO₃ (305 mg, 0.936 mmol) in dioxane (10 mL) and H₂O (2 mL) was stirred at 80° C. under N₂ for 12 hours. The reaction mixture was quenched with saturated NaCl aq. (10 mL), extracted with EA (30 mL×2), combined, washed brine (30 mL×2), dried and concentrated to dryness. The resulting residue was purified by flash column chromatography (DCM:MeOH=20:1) to give the titled compound (125 mg, 62%). MS: M/e 431 (M+1)⁺

### Step E: 3-fluoro-7-formyl-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide

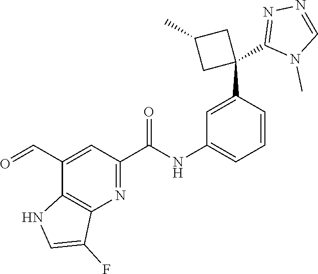 [SMILES: Cn1cnnc1[C@]1(c2cccc(NC(=O)c3cc(C=O)c4[nH]cc(F)c4n3)c2)C[C@H](C)C1]

To a solution of 3-fluoro-N-(3-((1s, 3s)-3-methyl-1-(4-methyl-4H-1,2,4-triazol-3-yl)cyclobutyl)phenyl)-7-vinyl-1H-pyrrolo[3,2-b]pyridine-5-carboxamide (125 mg, 0.291 mmol), K₂OsO₄ 2H₂O (11 mg, 0.0291 mmol) in dioxane (5 mL) and H₂O (1 mL) at 0° C. was added NaIO₄ (125 mg, 0.582 mmol). The mixture was stirred at room temperature for 4 hours. After filtration, the solution was concentrated to dryness. The resulting residue was purified by flash column chromatography (DCM:MeOH=15:1) to give the titled compound (80 mg, 63%). MS: M/e 433 (M+1)⁺

### Step E: 7-(((cyclobutylmethyl)amino)methyl)-3-fluoro-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide

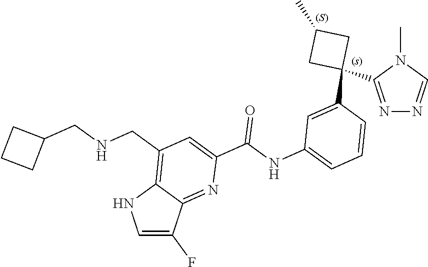

To a solution of 3-fluoro-7-formyl-N-(3-((1s, 3s)-3-methyl-1-(4-methyl-4H-1,2,4-triazol-3-yl)cyclobutyl)phenyl)-1H-pyrrolo[3,2-b]pyridine-5-carboxamide (20 mg, 0.046 mmol), cyclobutylmethanamine hydrochloride (11 mg, 0.093 mmol) and Et₃N (9 mg, 0.093 mmol) in DCM (1 ml) was stirred at room temperature for 1 hour, then added NaBH(OAc)₃ (40 mg, 0.186 mmol). The mixture solution was stirred at 25° C. for 36 hours. The reaction mixture was concentrated under reduced pressure. The crude product was purified by Prep-HPLC to give the title compound (10 mg, 43%). MS: M/e 502 (M+1). ¹H NMR (400 MHz, DMSO-d₆) δ 10.49 (s, 1H), 8.29 (s, 1H), 8.06 (s, 1H), 7.92 (s, 1H), 7.89 (d, J=8.3 Hz, 1H), 7.82 (s, 1H), 7.34 (t, J=7.9 Hz, 1H), 6.97 (d, J=7.7 Hz, 1H), 4.08 (s, 2H), 3.19 (s, 3H), 2.82 (d, J=6.0 Hz, 2H), 2.56 (dd, J=18.4, 7.9 Hz, 5H), 2.44 (dd, J=14.9, 7.4 Hz, 1H), 1.99 (d, J=8.0 Hz, 2H), 1.87-1.73 (m, 2H), 1.71-1.59 (m, 2H), 1.09 (d, J=5.3 Hz, 3H) ppm.

### Compound B37:7-((((S)-1-cyclopropylethyl)amino)methyl)-3-fluoro-N-(3-((1s,3R)-3-methyl-1-(4-methyl-4H-1,2,4-triazol-3-yl)cyclobutyl)phenyl)-1H-pyrrolo[3,2-b]pyridine-5-carboxamide

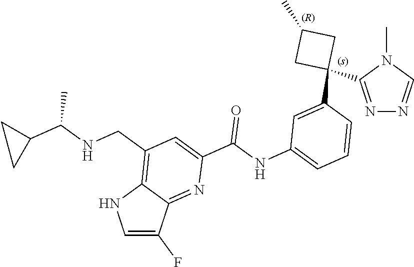

The compound B38 was synthesized starting from the corresponding starting materials according the similar procedures described as those of Compound B37 to give the title compound (9 mg, 35%). MS: M/e 502 (M+1)⁺. ¹H NMR (400 MHz, DMSO-d₆) δ 10.49 (s, 1H), 8.29 (s, 1H), 8.09 (s, 1H), 7.93 (s, 1H), 7.89 (d, J=8.1 Hz, 1H), 7.83 (s, 1H), 7.34 (t, J=7.8 Hz, 1H), 6.97 (d, J=7.6 Hz, 1H), 4.18 (s, 2H), 3.19 (s, 3H), 2.84 (s, 2H), 2.55 (t, J=11.9 Hz, 3H), 1.97-1.87 (m, 1H), 1.13 (d, J=6.1 Hz, 3H), 1.09 (d, J=4.2 Hz, 3H), 0.73 (d, J=5.0 Hz, 1H), 0.43 (s, 1H), 0.34 (s, 1H), 0.17 (s, 1H), 0.02 (s, 1H) ppm.

### Compound B38:7-((((S)-1-cyclobutylethyl)amino)methyl)-3-fluoro-N-(3-((1s,3R)-3-methyl-1-(4-methyl-4H-1,2,4-triazol-3-yl)cyclobutyl)phenyl)-1H-pyrrolo[3,2-b]pyridine-5-carboxamide

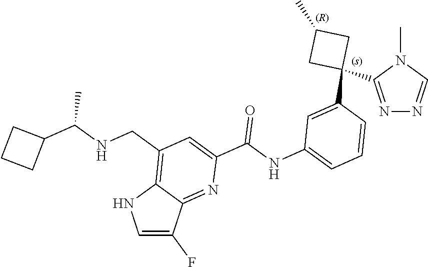

The compound B39 was synthesized starting from the corresponding starting materials according the similar procedures described as those of Compound B37 to give the title compound (8 mg, 33%). MS: M/e 516 (M+1)⁺. H NMR (400 MHz, DMSO) δ 10.49 (s, 1H), 8.29 (s, 1H), 8.08 (s, 1H), 7.95-7.87 (m, 2H), 7.82 (s, 1H), 7.34 (t, J=7.9 Hz, 1H), 6.97 (d, J=7.6 Hz, 1H), 4.07 (t, J=14.1 Hz, 2H), 3.19 (s, 3H), 2.84 (s, 2H), 2.55 (t, J=12.0 Hz, 3H), 2.46 (s, 1H), 2.26-2.16 (m, 1H), 1.99 (s, 1H), 1.87 (s, 1H), 1.80-1.60 (m, 4H), 1.09 (d, J=4.6 Hz, 3H), 0.92 (d, J=6.0 Hz, 3H) ppm.

### Compound B39: 4-{[(1-methylcyclobutyl)amino]methyl}-N-{3-[(1s, 3s)-3-(cyanomethyl)-1-(4-methyl-1,2,4-triazol-3-yl)cyclobutyl]phenyl}thieno[2,3-b]pyridine-6-carboxamide

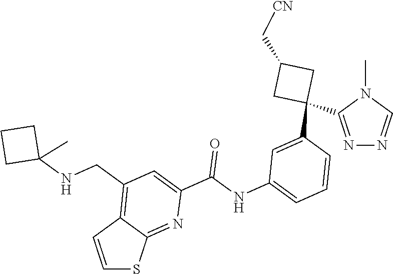 [SMILES: Cn1cnnc1[C@]1(c2cccc(NC(=O)c3cc(CNC4(C)CCC4)c4ccsc4n3)c2)C[C@H](CC#N)C1]

### Step 1: methyl 4-{[(1-methylcyclobutyl)amino]methyl}thieno[2,3-b]pyridine-6-carboxylate

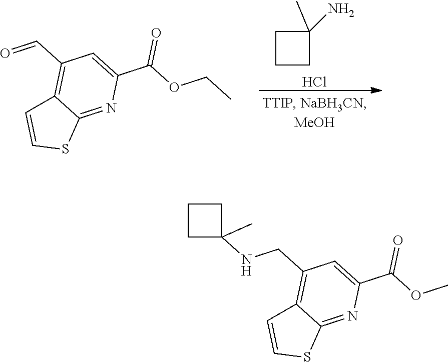

To a stirred mixture of ethyl 4-formylthieno[2,3-b]pyridine-6-carboxylate (25 mg, 0.091 mmol, 1 equiv, 85.5%) and 1-methylcyclobutan-1-amine hydrochloride (35 mg, 0.273 mmol, 3.01 equiv, 95%) in MeOH (2 mL) was added tetrakis(propan-2-yloxy)titanium (109 mg, 0.364 mmol, 4.01 equiv, 95%) dropwise at room temperature. The resulting mixture was stirred for 3 hrs at room temperature. To the above mixture was added NaBH₃CN (13 mg, 0.197 mmol, 2.16 equiv, 95%) in portions. The resulting mixture was stirred for additional 3 hrs at room temperature. The resulting mixture was concentrated under vacuum. The residue was purified by silica gel column chromatography, eluted with PE/EA (1:1) to afford methyl 4-{[(1-methylcyclobutyl)amino]methyl}thieno[2,3-b]pyridine-6-carboxylate (19 mg, 50.77%). MS: M/e 291 (M+1)⁺

### Step 2: 4-{[(1-methylcyclobutyl)amino]methyl}thieno[2,3-b]pyridine-6-carboxylic acid

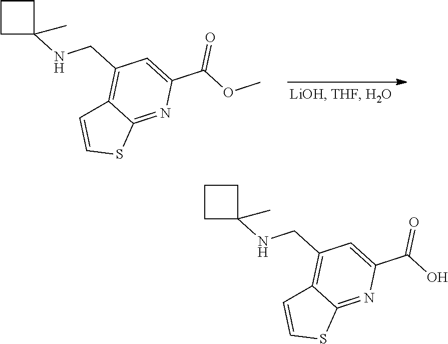 [SMILES: COC(=O)c1cc(CNC2(C)CCC2)c2ccsc2n1 ; CC1(NCc2cc(C(=O)O)nc3sccc23)CCC1]

To a stirred solution of methyl 4-{[(1-methylcyclobutyl)amino]methyl}thieno[2,3-b]pyridine-6-carboxylate (19 mg, 0.046 mmol, 1 equiv, 70.5%) in THF (1.5 mL, 99%) and H₂O (0.5 mL, 99%) were added LiOH (3 mg, 0.119 mmol, 2.58 equiv, 95%) in portions. The resulting mixture was stirred for 4 hrs at room temperature. The mixture/residue was acidified to pH=4 with HCl (1 M). The resulting mixture was concentrated under vacuum. The residue was purified by reversed-phase flash chromatography with the following conditions: column, C18 silica gel; mobile phase, MeCN in Water (0.1% FA), 0% to 95% gradient in 25 min; detector, UV 254 nm. This resulted in 4-{[(1-methylcyclobutyl)amino]methyl}thieno[2,3-b]pyridine-6-carboxylic acid (11 mg, 85.43%). MS: M/e 277 (M+1)⁺.

### Step 3: 4-{[(1-methylcyclobutyl)amino]methyl}-N-{3-[(1s, 3s)-3-(cyanomethyl)-1-(4-methyl-1,2,4-triazol-3-yl)cyclobutyl]phenyl}thieno[2,3-b]pyridine-6-carboxamide

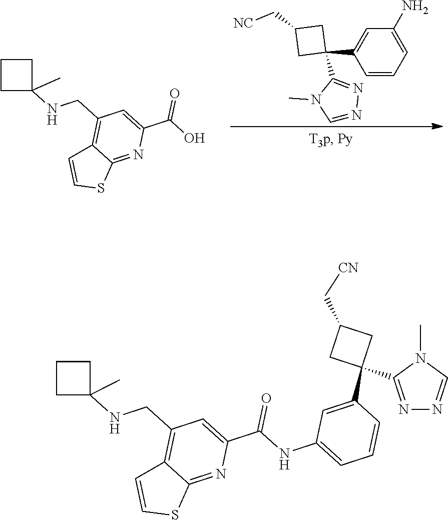 [SMILES: CC1(NCc2cc(C(=O)O)nc3sccc23)CCC1 ; Cn1cnnc1[C@]1(c2cccc(N)c2)C[C@H](CC#N)C1 ; Cn1cnnc1[C@]1(c2cccc(NC(=O)c3cc(CNC4(C)CCC4)c4ccsc4n3)c2)C[C@H](CC#N)C1]

Into a 10 mL sealed tube were added 4-{[(1-methylcyclobutyl)amino]methyl}thieno[2,3-b]pyridine-6-carboxylic acid (10 mg, 0.036 mmol, 1 equiv, 99.0%) and 2-[(1s, 3s)-3-(3-aminophenyl)-3-(4-methyl-1,2,4-triazol-3-yl)cyclobutyl]acetonitrile (10 mg, 0.037 mmol, 1.03 equiv, 98.3%) and Pyridine (1 mL, 95%). The resulting mixture was stirred for 5 min at room temperature. To the above mixture was added T₃P (114 mg, 0.179 mmol, 5.00 equiv, 50%) dropwise at room temperature. The resulting mixture was stirred for additional 12 hrs at room temperature. The resulting mixture was concentrated under vacuum. The crude product (50 mg) was purified by Prep-HPLC with the following conditions (2 #SHIMADZU (HPLC-01)): Column, XBridge Prep OBD C18 Column, 30*150 mm, 5 μm; mobile phase, 10 mmol NH₄HCO₃+0.05% NH₃H₂O and ACN (30% ACN up to 60% in 8 min); This resulted in 4-{[(1-methylcyclobutyl)amino]methyl}-N-{3-[(1s, 3s)-3-(cyanomethyl)-1-(4-methyl-1,2,4-triazol-3-yl)cyclobutyl]phenyl}thieno[2,3-b]pyridine-6-carboxamide (10.1 mg, 53.52%). MS: M/e 526 (M+1)⁺. ¹H NMR (300 MHz, DMSO-d₆) δ 10.77 (s, 1H), 8.32 (d, J=6.2 Hz, 2H), 8.11 (d, J=6.1 Hz, 1H), 8.04-7.94 (m, 2H), 7.75 (d, J=6.1 Hz, 1H), 7.45-7.34 (m, 1H), 7.04-6.97 (m, 1H), 4.09 (s, 2H), 3.22 (s, 3H), 2.94-2.67 (m, 7H), 2.12-1.97 (m, 2H), 1.84-1.64 (m, 4H), 1.29 (s, 3H).

### Compound B40: 7-{[(cyclobutylmethyl)amino]methyl}-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide

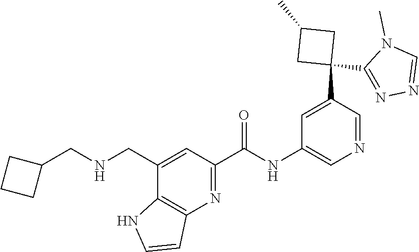 [SMILES: Cn1cnnc1[C@]1(c2cncc(NC(=O)c3cc(CNCC4CCC4)c4[nH]ccc4n3)c2)C[C@H](C)C1]

### Step 1: 7-{[(cyclobutylmethyl)amino]methyl}-1H-pyrrolo[3,2-b]pyridine-5-carbonitrile

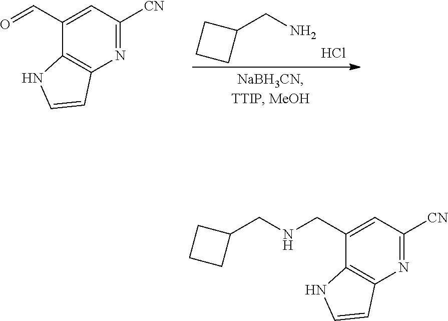 [SMILES: N#Cc1cc(C=O)c2[nH]ccc2n1 ; NCC1CCC1 ; Cl ; N#Cc1cc(CNCC2CCC2)c2[nH]ccc2n1]

To a stirred solution of 7-formyl-1H-pyrrolo[3,2-b]pyridine-5-carbonitrile (100 mg, 0.538 mmol, 1 equiv, 92.0%) and 1-cyclobutylmethanamine hydrochloride (137.61 mg, 1.076 mmol, 2 equiv, 95%) in MeOH (3 mL, 100%) were added AcOH (67.96 mg, 1.076 mmol, 2 equiv, 95%) at room temperature. The resulting mixture was stirred for 1 h at room temperature. To the above mixture was added NaBH₃CN (71.11 mg, 1.076 mmol, 2 equiv, 95%) at room temperature. The resulting mixture was stirred for additional 2 hrs at room temperature. The resulting mixture was concentrated under reduced pressure. The residue was purified by silica gel column chromatography, eluted with PE/EA (1:1) to afford 7-{[(cyclobutylmethyl)amino]methyl}-1H-pyrrolo[3,2-b]pyridine-5-carbonitrile (80 mg, 55.74%). MS: M/e 241 (M+1)⁺.

### Step 2: 7-{[(cyclobutylmethyl)amino]methyl}-1H-pyrrolo[3,2-b]pyridine-5-carboxylic acid

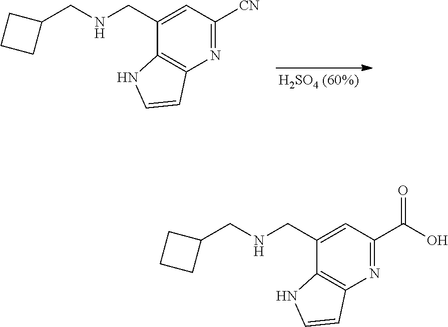 [SMILES: N#Cc1cc(CNCC2CCC2)c2[nH]ccc2n1 ; O=C(O)c1cc(CNCC2CCC2)c2[nH]ccc2n1]

A solution of 7-{[(cyclobutylmethyl)amino]methyl}-1H-pyrrolo[3,2-b]pyridine-5-carbonitrile (80 mg, 0.300 mmol, 1 equiv, 90%) in H₂SO₄ (3 mL, 33.772 mmol, 112.72 equiv, 60%) was stirred for 1 h at 100° C. under nitrogen atmosphere. The mixture was allowed to cool down to 0° C. The mixture was neutralized to pH=3 with NaOH (2N). The resulting mixture was concentrated under vacuum. The resulting mixture was diluted with MeOH (5 mL). The resulting mixture was filtered, the filter cake was washed with MeOH (3×5 mL). The filtrate was concentrated under reduced pressure. The residue was purified by reverse flash chromatography with the following conditions: column, C18 silica gel; mobile phase, MeCN in Water (0.1% TFA), 0% to 10% gradient in 10 min; detector, UV 220 m. This resulted in 7-{[(cyclobutylmethyl)amino]methyl}-1H-pyrrolo[3,2-b]pyridine-5-carboxylic acid (60 mg, 69.50%). MS: M/e 260 (M+1)⁺.

### Step 3: 7-{[(cyclobutylmethyl)amino]methyl}-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide

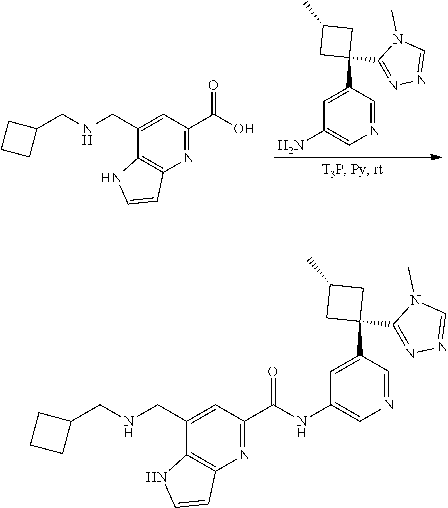 [SMILES: O=C(O)c1cc(CNCC2CCC2)c2[nH]ccc2n1 ; Cn1cnnc1[C@]1(c2cncc(N)c2)C[C@H](C)C1 ; Cn1cnnc1[C@]1(c2cncc(NC(=O)c3cc(CNCC4CCC4)c4[nH]ccc4n3)c2)C[C@H](C)C1]

To a stirred solution of 7-{[(cyclobutylmethyl)amino]methyl}-1H-pyrrolo[3,2-b]pyridine-5-carboxylic acid (20 mg, 0.069 mmol, 1 equiv, 90.0%) and 5-[(1r, 3s)-3-methyl-1-(4-methyl-1,2,4-triazol-3-yl)cyclobutyl]pyridin-3-amine (17.78 mg, 0.069 mmol, 1 equiv, 95%) in Pyridine (3 mL, 100%) was added T₃P (69.75 mg, 0.207 mmol, 3 equiv, 95%) at room temperature. The resulting mixture was stirred for 3 hrs at room temperature. The resulting mixture was concentrated under vacuum. The crude product was purified by Chiral-Prep-HPLC with the following conditions (2 #SHIMADZU (HPLC-01)): Column, XBridge Prep OBD C18 Column, 30*150 mm, 5 μm; mobile phase, 10 mmol NH₄HCO₃+0.05% NH₃H2O and ACN (25% ACN up to 55% in 8 min); This resulted in 7-{[(cyclobutylmethyl)amino]methyl}-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide (13.0 mg, 37.68%). MS: M/e 485 (M+1)⁺. ¹H NMR (300 MHz, DMSO-d₆) δ 10.81 (s, 1H), 9.11 (d, J=2.3 Hz, 1H), 8.33 (s, 2H), 8.23 (d, J=2.1 Hz, 1H), 8.02 (s, 1H), 7.79 (d, J=3.3 Hz, 1H), 6.74 (d, J=3.3 Hz, 1H), 4.08 (s, 2H), 3.23 (s, 3H), 2.94-2.83 (m, 2H), 2.68-2.51 (m, 5H), 2.50-2.35 (m, 1H), 2.08-1.92 (m, 2H), 1.91-1.78 (m, 2H), 1.75-1.55 (m, 2H), 1.10 (d, J=5.7 Hz, 3H) ppm.

### Compound B41: 4-{[(cyclobutylmethyl)amino]methyl}-N-{3-[(1r, 3s)-3-methyl-1-(4-methyl-1,2,4-triazol-3-yl)cyclobutyl]phenyl}thieno[2,3-b]pyridine-6-carboxamide

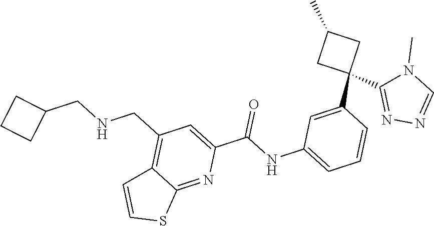 [SMILES: Cn1cnnc1[C@]1(c2cccc(NC(=O)c3cc(CNCC4CCC4)c4ccsc4n3)c2)C[C@H](C)C1]

### Step 1: ethyl 4-{[(cyclobutylmethyl)amino]methyl}thieno[2,3-b]pyridine-6-carboxylate

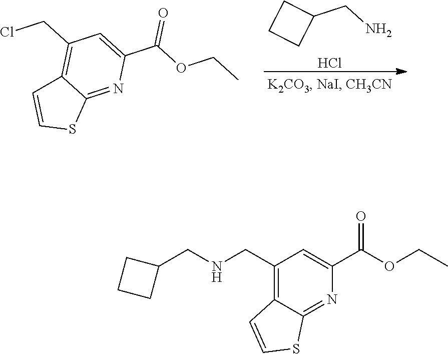 [SMILES: CCOC(=O)c1cc(CCl)c2ccsc2n1 ; NCC1CCC1 ; Cl ; CCOC(=O)c1cc(CNCC2CCC2)c2ccsc2n1]

Into a 10 mL sealed tube were added ethyl 4-(chloromethyl)thieno[2,3-b]pyridine-6-carboxylate (70 mg, 0.261 mmol, 1 equiv, 95.4%), NaI (42 mg, 0.266 mmol, 1.02 equiv, 95%), K₂CO₃ (76 mg, 0.522 mmol, 2.00 equiv, 95%) and CH₃CN (2 mL, 99%). The resulting mixture was stirred for 3 hrs at 70° C. under nitrogen atmosphere. The mixture was allowed to cool down to room temperature. The resulting mixture was concentrated under vacuum. The residue was purified by silica gel column chromatography, eluted with PE/EA (1:1) to afford ethyl 4-{[(cyclobutylmethyl)amino]methyl}thieno[2,3-b]pyridine-6-carboxylate (73 mg, 90.91%). MS: M/e 305 (M+1)⁺.

### Step 2: 4-{[(cyclobutylmethyl)amino]methyl}thieno[2,3-b]pyridine-6-carboxylic acid

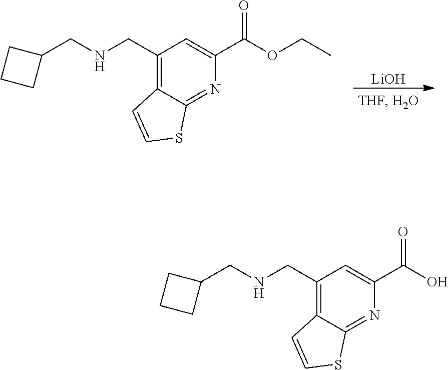

To a stirred solution of ethyl 4-{[(cyclobutylmethyl)amino]methyl}thieno[2,3-b]pyridine-6-carboxylate (72 mg, 0.234 mmol, 1 equiv, 99.0%) in THF (1 mL, 99%) and H₂O (0.5 mL, 99%) were added LiOH (12 mg, 0.476 mmol, 2.03 equiv, 95%). The resulting mixture was stirred for 4 hrs at room temperature. The mixture was acidified to pH=4 with HCl (1 M). The resulting mixture was concentrated under vacuum. The residue was purified by reversed-phase flash chromatography with the following conditions: column, C18 silica gel; mobile phase, MeCN in Water (0.1% FA), 0% to 95% gradient in 30 min; detector, UV 254 nm. This resulted in 4-{[(cyclobutylmethyl)amino]methyl}thieno[2,3-b]pyridine-6-carboxylic acid (60 mg, 91.79%). MS: M/e 277 (M+1)⁺.

### Step 3: 4-{[(cyclobutylmethyl)amino]methyl}-N-{3-[(1r, 3s)-3-methyl-1-(4-methyl-1,2,4-triazol-3-yl)cyclobutyl]phenyl}thieno[2,3-b]pyridine-6-carboxamide

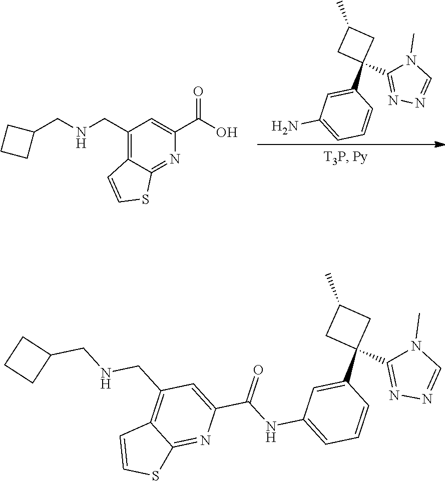 [SMILES: O=C(O)c1cc(CNCC2CCC2)c2ccsc2n1 ; Cn1cnnc1[C@]1(c2cccc(N)c2)C[C@H](C)C1 ; Cn1cnnc1[C@]1(c2cccc(NC(=O)c3cc(CNCC4CCC4)c4ccsc4n3)c2)C[C@H](C)C1]

Into a 10 mL sealed tube were added 4-{[(cyclobutylmethyl)amino]methyl}thieno[2,3-b]pyridine-6-carboxylic acid (25 mg, 0.090 mmol, 1 equiv, 99.0%) and 3-[(1r, 3s)-3-methyl-1-(4-methyl-1,2,4-triazol-3-yl)cyclobutyl]aniline (27 mg, 0.091 mmol, 1.02 equiv, 81.8%) and Pyridine (1 mL, 95%). The resulting mixture was stirred for 5 min at room temperature. To the above mixture was added T₃P (285 mg, 0.448 mmol, 5.00 equiv, 50%) dropwise. The resulting mixture was stirred for additional 3 hrs at room temperature. The resulting mixture was concentrated under vacuum. The crude product (100 mg) was purified by Prep-HPLC with the following conditions (2 #SHIMADZU (HPLC-01)): Column, Xselect CSH C18 OBD Column 30*150 mm 5 um, n; mobile phase, Water (10 mmol/L NH₄HCO₃+0.1% NH₃·H₂O) and ACN (20% ACN up to 50% in 8 min); This resulted in 4-{[(cyclobutylmethyl)amino]methyl}-N-{3-[(1r, 3s)-3-methyl-1-(4-methyl-1,2,4-triazol-3-yl)cyclobutyl]phenyl}thieno[2,3-b]pyridine-6-carboxamide (9.1 mg, 20%). MS: M/e 501 (M+1)⁺. ¹H NMR (300 MHz, DMSO-d₆) δ 10.71 (s, 1H), 8.27 (d, J=14.0 Hz, 2H), 8.09 (d, J=6.0 Hz, 1H), 7.98-7.90 (m, 2H), 7.73 (d, J=6.1 Hz, 1H), 7.39-7.31 (m, 1H), 7.02-6.95 (m, 1H), 4.14 (s, 2H), 3.20 (s, 3H), 2.88-2.78 (m, 2H), 2.64-2.52 (m, 5H), 2.47-2.38 (m, 1H), 2.06-1.94 (m, 2H), 1.90-1.74 (m, 2H), 1.72-1.59 (m, 2H), 1.09 (d, J=5.7 Hz, 3H).

### Compound B42: 4-({[(1S)-1-cyclobutylethyl]amino}methyl)-N-{5-[(1r, 3s)-3-methyl-1-(4-methyl-1,2,4-triazol-3-yl)cyclobutyl]pyridin-3-yl}thieno[2,3-b]pyridine-6-carboxamide

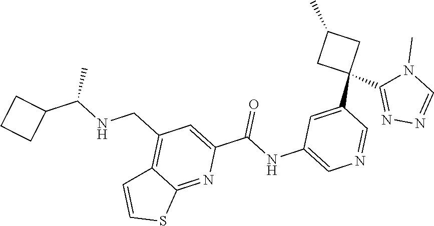 [SMILES: C[C@H](NCc1cc(C(=O)Nc2cncc([C@]3(c4nncn4C)C[C@@H](C)C3)c2)nc2sccc12)C1CCC1]

### Step 1: ethyl 4-({[(1S)-1-cyclobutylethyl]amino}methyl)thieno[2,3-b]pyridine-6-carboxylate

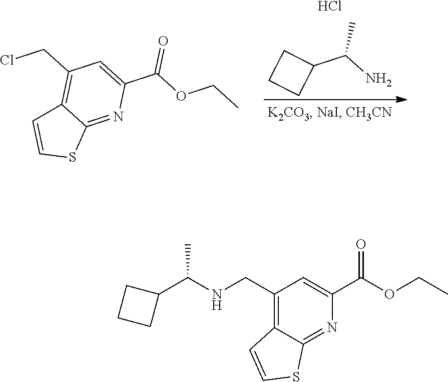

Into a 8 mL sealed tube were added ethyl 4-(chloromethyl)thieno[2,3-b]pyridine-6-carboxylate (25 mg, 0.093 mmol, 1 equiv, 95.4%), NaI (15 mg, 0.095 mmol, 1.02 equiv, 95%), K₂CO₃ (28 mg, 0.192 mmol, 2.06 equiv, 95%), (1S)-1-cyclobutylethanamine hydrochloride (27 mg, 0.189 mmol, 2.03 equiv, 95%) and CH₃CN (1.5 mL, 99%). The resulting mixture was stirred for 2 h at 70° C. The mixture was allowed to cool down to room temperature. The resulting mixture was concentrated under vacuum. The residue was purified by silica gel column chromatography, eluted with PE/EA (1:1) to afford ethyl 4-({[(1S)-1-cyclobutylethyl]amino}methyl)thieno[2,3-b]pyridine-6-carboxylate (26 mg, 70.91%) as a off-white solid. LC-MS (M+H)⁺:=319.

### Step 2: 4-({[(1S)-1-cyclobutylethyl]amino}methyl)thieno[2,3-b]pyridine-6-carboxylic acid

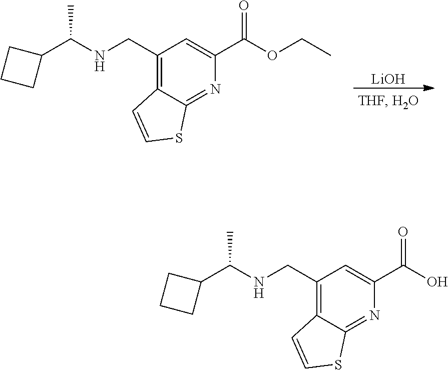

To a stirred solution of ethyl 4-({[(1S)-1-cyclobutylethyl]amino}methyl)thieno[2,3-b]pyridine-6-carboxylate (26 mg, 0.066 mmol, 1 equiv, 81.0%) in THF (1.5 mL, 99%) and H₂O (0.5 mL, 99%) was added LiOH (4 mg, 0.159 mmol, 2.40 equiv, 95%) in portions. The resulting mixture was stirred for 3 h at room temperature. The mixture was acidified to pH 3 with HCl (1 M). The resulting mixture was concentrated under vacuum. The residue was purified by reversed-phase flash chromatography with the following conditions: column, C18 silica gel; mobile phase, MeCN in Water (0.1% FA), 0% to 40% gradient in 30 min; This resulted in 4-({[(1S)-1-cyclobutylethyl]amino}methyl)thieno[2,3-b]pyridine-6-carboxylic acid (22 mg, 97.37%) as a off-white solid. LC-MS (M+H)⁺:=291.

### Step 3: 4-({[(1S)-1-cyclobutylethyl]amino}methyl)-N-{5-[(1r, 3s)-3-methyl-1-(4-methyl-1,2,4-triazol-3-yl)cyclobutyl]pyridin-3-yl}thieno[2,3-b]pyridine-6-carboxamide

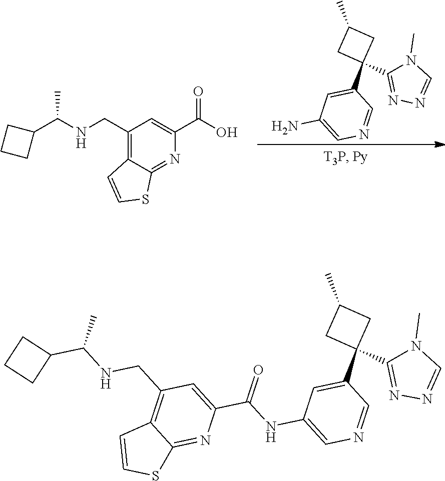 [SMILES: C[C@H](NCc1cc(C(=O)O)nc2sccc12)C1CCC1 ; Cn1cnnc1[C@]1(c2cncc(N)c2)C[C@H](C)C1 ; C[C@H](NCc1cc(C(=O)Nc2cncc([C@]3(c4nncn4C)C[C@@H](C)C3)c2)nc2sccc12)C1CCC1]

To a stirred mixture of 4-({[(1S)-1-cyclobutylethyl]amino}methyl)thieno[2,3-b]pyridine-6-carboxylic acid (20 mg, 0.059 mmol, 1 equiv, 85.0%) and 5-[(1r, 3s)-3-methyl-1-(4-methyl-1,2,4-triazol-3-yl)cyclobutyl]pyridin-3-amine (15 mg, 0.059 mmol, 1.00 equiv, 95%) in Pyridine (1.5 mL, 99%) were added T₃P (299 mg, 0.470 mmol, 8.03 equiv, 50%) dropwise at room temperature. The resulting mixture was stirred for 1 h at room temperature. The resulting mixture was concentrated under vacuum. The crude product (150 mg) was purified by Prep-HPLC with the following conditions (2 #SHIMADZU (HPLC-01)): Column, XBridge Prep OBD C18 Column, 30*150 mm, 5 μm; mobile phase, 10 mmol NH₄HCO₃+0.05% NH₃H2O and ACN (32% ACN up to 62% in 8 min); This resulted in 4-({[(1S)-1-cyclobutylethyl]amino}methyl)-N-{5-[(1r, 3s)-3-methyl-1-(4-methyl-1,2,4-triazol-3-yl)cyclobutyl]pyridin-3-yl}thieno[2,3-b]pyridine-6-carboxamide (15.8 mg, 51.13%). LC-MS (M+H)⁺:=516; ¹H NMR (400 MHz, DMSO-d6) δ 11.00 (s, 1H), 9.14 (d, J=2.2 Hz, 1H), 8.35 (s, 1H), 8.33-8.26 (m, 3H), 8.12 (d, J=6.0 Hz, 1H), 7.75 (d, J=6.0 Hz, 1H), 4.23-4.09 (m, 2H), 3.23 (s, 3H), 2.92-2.84 (m, 2H), 2.66-2.52 (m, 4H), 2.30-2.15 (m, 1H), 2.05-1.94 (m, 1H), 1.94-1.84 (m, 1H), 1.84-1.61 (m, 4H), 1.10 (d, J=5.6 Hz, 3H), 0.94 (d, J=6.2 Hz, 3H) ppm.

### Compound B43: 7-({[(1S)-1-cyclobutylethyl]amino}methyl)-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide

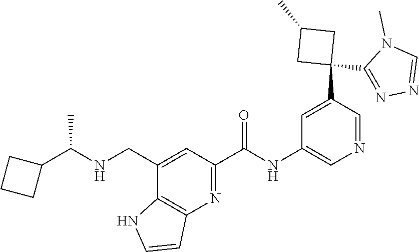 [SMILES: C[C@H](NCc1cc(C(=O)Nc2cncc([C@]3(c4nncn4C)C[C@@H](C)C3)c2)nc2cc[nH]c12)C1CCC1]

### Step 1: 7-({[(1S)-1-cyclobutylethyl]amino}methyl)-1H-pyrrolo[3,2-b]pyridine-5-carbonitrile

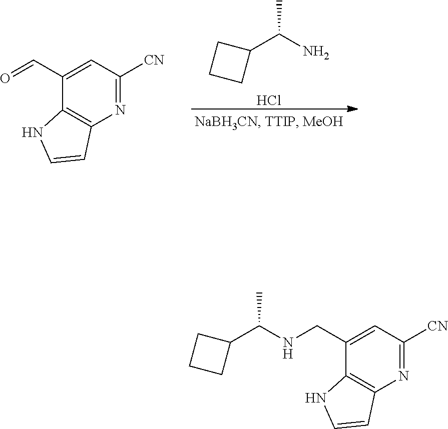

To a stirred solution of 7-formyl-1H-pyrrolo[3,2-b]pyridine-5-carbonitrile (50 mg, 0.269 mmol, 1 equiv, 92.0%) and (1S)-1-cyclobutylethanamine hydrochloride (76.75 mg, 0.538 mmol, 2 equiv, 95%) in MeOH (6 mL, 100%) were added tetrakis(propan-2-yloxy)titanium (321.62 mg, 1.076 mmol, 4 equiv, 95%) at room temperature. The resulting mixture was stirred for 1 h at room temperature. To the above mixture was added NaBH₃CN (35.55 mg, 0.538 mmol, 2 equiv, 95%) at room temperature. The resulting mixture was stirred for additional 2 hrs at room temperature. The resulting mixture was concentrated under reduced pressure. The residue was purified by silica gel column chromatography, eluted with PE/EA (1:1) to afford 7-({[(1S)-1-cyclobutylethyl]amino}methyl)-1H-pyrrolo[3,2-b]pyridine-5-carbonitrile (30 mg, 41.69%). MS: M/e 255 (M+H)⁺.

### step 2: 7-({[(1S)-1-cyclobutylethyl]amino}methyl)-1H-pyrrolo[3,2-b]pyridine-5-carboxylic acid

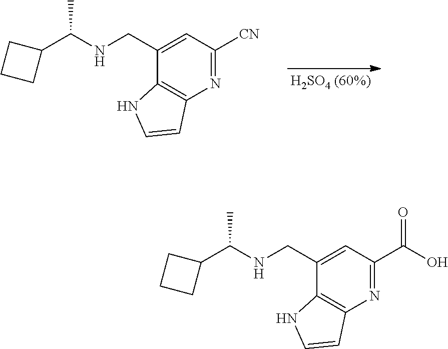 [SMILES: C[C@H](NCc1cc(C#N)nc2cc[nH]c12)C1CCC1 ; C[C@H](NCc1cc(C(=O)O)nc2cc[nH]c12)C1CCC1]

A solution of 7-({[(1S)-1-cyclobutylethyl]amino}methyl)-1H-pyrrolo[3,2-b]pyridine-5-carbonitrile (30 mg, 0.112 mmol, 1 equiv, 95.0%) in H₂SO₄ (1.00 mL, 11.252 mmol, 100.46 equiv, 60%) was stirred for 1 h at 100° C. The mixture was allowed to cool down to 0° C. The mixture was neutralized to pH=3 with NaOH (2N). The resulting mixture was concentrated under vacuum. The resulting mixture was diluted with MeOH (5 mL). The resulting mixture was filtered, the filter cake was washed with MeOH (3×5 mL). The filtrate was concentrated under reduced pressure. The residue was purified by reverse flash chromatography with the following conditions: column, C18 silica gel; mobile phase, MeCN in Water (0.1% TFA), 0% to 10% gradient in 10 min; This resulted in 7-({[(1S)-1-cyclobutylethyl]amino}methyl)-1H-pyrrolo[3,2-b]pyridine-5-carboxylic acid (20 mg, 62.03%). MS: M/e 274 (M+H)⁺.

### step 3: 7-({[(1S)-1-cyclobutylethyl]amino}methyl)-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide

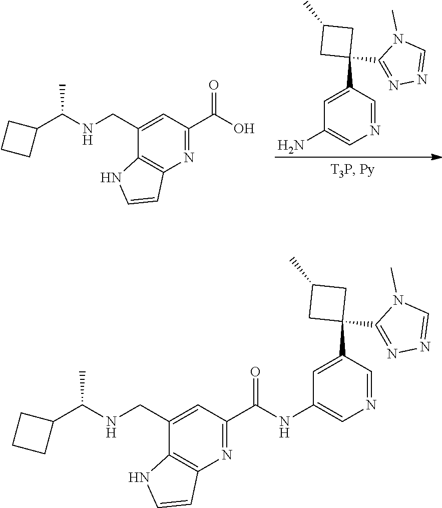 [SMILES: C[C@H](NCc1cc(C(=O)O)nc2cc[nH]c12)C1CCC1 ; Cn1cnnc1[C@]1(c2cncc(N)c2)C[C@H](C)C1 ; C[C@H](NCc1cc(C(=O)Nc2cncc([C@]3(c4nncn4C)C[C@@H](C)C3)c2)nc2cc[nH]c12)C1CCC1]

To a stirred solution of 7-({[(1S)-1-cyclobutylethyl]amino}methyl)-1H-pyrrolo[3,2-b]pyridine-5-carboxylic acid (20 mg, 0.070 mmol, 1 equiv, 95.0%) and 5-[(1r, 3s)-3-methyl-1-(4-methyl-1,2,4-triazol-3-yl)cyclobutyl]pyridin-3-amine (14.24 mg, 0.056 mmol, 0.8 equiv, 95.0%) in Pyridine (1 mL, 100%) was added T₃P (69.84 mg, 0.210 mmol, 3 equiv, 95%) at room temperature. The resulting mixture was stirred for 3 hrs at room temperature. The resulting mixture was concentrated under vacuum. The crude product was purified by Chiral-Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (27% ACN up to 57% in 9 min); This resulted in 7-({[(1S)-1-cyclobutylethyl]amino}methyl)-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide (5.4 mg, 15.10%). MS: M/e 499 (M+H)⁺. ¹H NMR (300 MHz, DMSO-d₆) δ 10.83 (s, 1H), 9.11 (s, 1H), 8.33 (d, J=2.7 Hz, 2H), 8.23 (s, 1H), 8.04 (s, 1H), 7.81 (d, J=3.3 Hz, 1H), 6.74 (d, J=3.3 Hz, 1H), 4.21-4.01 (m, 2H), 3.23 (s, 3H), 2.87 (s, 2H), 2.59 (d, J=7.7 Hz, 3H), 2.51-2.40 (m, 1H), 2.27-2.15 (m, 1H), 2.06-1.94 (m, 1H), 1.93-1.82 (m, 1H), 1.81-1.55 (m, 4H), 1.10 (d, J=5.7 Hz, 3H), 0.93 (d, J=6.2 Hz, 3H) ppm.

### Compound B44: N-(5-((1s, 3s)-3-methyl-1-(4-methyl-4H-1,2,4-triazol-3-yl)cyclobutyl)pyridin-3-yl)-7-(((1-methylcyclopropyl)amino)methyl)-1H-pyrrolo[3,2-b]pyridine-5-carboxamide

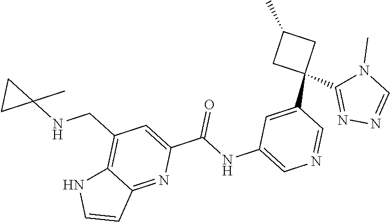 [SMILES: Cn1cnnc1[C@]1(c2cncc(NC(=O)c3cc(CNC4(C)CC4)c4[nH]ccc4n3)c2)C[C@H](C)C1]

### Step 1: 7-(((1-methylcyclopropyl)amino)methyl)-1H-pyrrolo[3,2-b]pyridine-5-carbonitrile

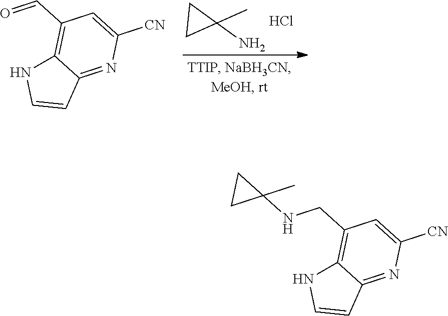

To a stirred solution of 7-formyl-1H-pyrrolo[3,2-b]pyridine-5-carbonitrile (100 mg, 0.555 mmol, 1 equiv, 95%) and 1-methylcyclopropan-1-amine hydrochloride (125.71 mg, 1.110 mmol, 2 equiv, 95%) in MeOH (5 ml) was added tetrakis(propan-2-yloxy)titanium (664.21 mg, 2.220 mmol, 4 equiv, 95%) at 0° C. under nitrogen atmosphere. The resulting mixture was stirred for additional 1 h at room temperature. To the above mixture was added NaBH₃CN (73.43 mg, 1.110 mmol, 2 equiv, 95%). The resulting mixture was stirred for additional 1 h at room temperature. The resulting mixture was concentrated under reduced pressure. The residue was purified by reversed-phase flash chromatography with the following conditions: column, C18 silica gel; mobile phase, MeOH in Water (0.1% TFA), 0% to 100% gradient in 30 min; detector, UV 254 nm. This resulted in 7-(((1-methylcyclopropyl)amino)methyl)-1H-pyrrolo[3,2-b]pyridine-5-carbonitrile (85 mg, 60.91%). MS: M/e 227 (M+H)⁺.

### Step 2: 7-(((1-methylcyclopropyl)amino)methyl)-1H-pyrrolo[3,2-b]pyridine-5-carboxylic acid

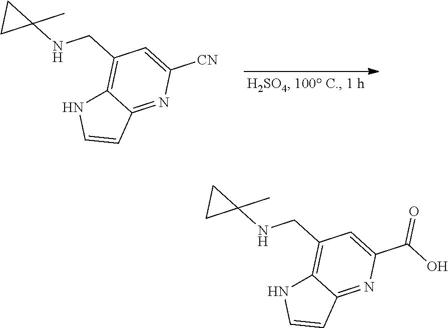 [SMILES: CC1(NCc2cc(C#N)nc3cc[nH]c23)CC1 ; CC1(NCc2cc(C(=O)O)nc3cc[nH]c23)CC1]

A solution of 7-(((1-methylcyclopropyl)amino)methyl)-1H-pyrrolo[3,2-b]pyridine-5-carbonitrile (85 mg, 0.357 mmol, 1 equiv, 95%) and H₂SO₄ (3 mL, 33.772 mmol, 94.64 equiv, 60%) was stirred for 1 h at 100° C. under nitrogen atmosphere. The mixture was basified to pH=5 with NaOH. The resulting mixture was concentrated under vacuum. The residue was purified by reversed-phase flash chromatography with the following conditions: column, C18 silica gel; mobile phase, MeCN in Water (0.1% TFA), 0% to 100% gradient in 30 min; detector, UV 254 nm. This resulted in 7-(((1-methylcyclopropyl)amino)methyl)-1H-pyrrolo[3,2-b]pyridine-5-carboxylic acid (45 mg, 51.41%). MS: M/e 246 (M+H)⁺.

### Step 3: N-(5-((1s, 3s)-3-methyl-1-(4-methyl-4H-1,2,4-triazol-3-yl)cyclobutyl)pyridin-3-yl)-7-(((1-methylcyclopropyl)amino)methyl)-1H-pyrrolo[3,2-b]pyridine-5-carboxamide

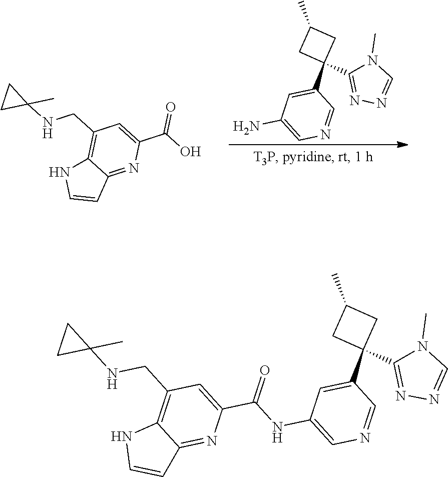 [SMILES: CC1(NCc2cc(C(=O)O)nc3cc[nH]c23)CC1 ; Cn1cnnc1[C@]1(c2cncc(N)c2)C[C@H](C)C1 ; Cn1cnnc1[C@]1(c2cncc(NC(=O)c3cc(CNC4(C)CC4)c4[nH]ccc4n3)c2)C[C@H](C)C1]

To a stirred solution of 7-(((1-methylcyclopropyl)amino)methyl)-1H-pyrrolo[3,2-b]pyridine-5-carboxylic acid (45 mg, 0.174 mmol, 1 equiv, 95%) and 5-[(1r, 3s)-3-methyl-1-(4-methyl-1,2,4-triazol-3-yl)cyclobutyl]pyridin-3-amine (53.57 mg, 0.209 mmol, 1.2 equiv, 95%) in pyridine (3 ml) was added tetrakis(propan-2-yloxy)titanium (521.43 mg, 1.740 mmol, 10 equiv, 95%) at 0° C. under nitrogen atmosphere. The resulting mixture was stirred for additional 1 h at room temperature. The crude product was purified by Chiral-Prep-HPLC with the following conditions (2 #SHIMADZU (HPLC-01)): Column: XBridge Prep OBD C18 Column, 30*150 mm, 5 μm; mobile phase, 10 mmol NH₄HCO₃+0.1% NH3H2O and ACN (25% ACN up to 55% in 9 min); This resulted in N-(5-((1s, 3s)-3-methyl-1-(4-methyl-4H-1,2,4-triazol-3-yl)cyclobutyl)pyridin-3-yl)-7-(((1-methylcyclopropyl)amino)methyl)-1H-pyrrolo[3,2-b]pyridine-5-carboxamide (17.6 mg, 21.46%). MS: M/e 471 (M+H)⁺. ¹H NMR (400 MHz, DMSO-d₆) δ 11.57 (s, 1H), 10.83 (s, 1H), 9.11 (m, 1H), 8.33 (m, 2H), 8.23 (m, 1H), 8.01 (s, 1H), 7.79 (m, 1H), 6.73 (m, 1H), 4.14 (s, 2H), 3.23 (s, 3H), 2.88 (m, 2H), 2.58 (m, 3H), 1.30 (s, 3H), 1.10 (m, 3H), 0.59 (m, 2H), 0.37-0.30 (m, 2H).

### Compound B45: 4-(((cyclobutylmethyl)amino)methyl)-1-methyl-N-(3-((1s, 3s)-3-methyl-1-(4-methyl-4H-1,2,4-triazol-3-yl)cyclobutyl)phenyl)-1H-pyrrolo[2,3-b]pyridine-6-carboxamide

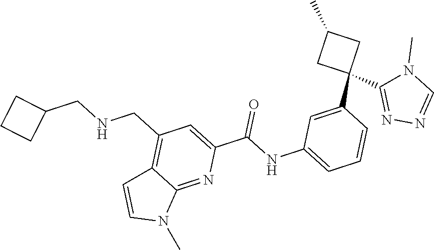 [SMILES: Cn1ccc2c(CNCC3CCC3)cc(C(=O)Nc3cccc([C@]4(c5nncn5C)C[C@@H](C)C4)c3)nc21]

### Step 1: 4-chloro-1H-pyrrolo[2,3-b]pyridine 7-oxide

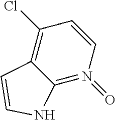 [SMILES: [O-][n+]1ccc(Cl)c2cc[nH]c21]

To s solution of 4-chloro-1H-pyrrolo[2,3-b]pyridine (10 g, 65.54 mmol) in EtOAc (500 mL) was added m-CPBA (16.96 g, 78.65 mmol, 80% purity) at 0° C., then the mixture was stirred at 25° C. for 16 h. The reaction mixture was quenched by the addition of saturated aqueous Na₂SO₃ (200 mL), then the solution was filtered and concentrated under reduced pressure to give a residue. The crude product was used for next step directly without purification. MS(ESI) m/e [M+1]169.0

### Step 2: 4-chloro-7-methoxy-1H-pyrrolo[2,3-b]pyridin-7-ium

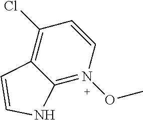

A mixture of 4-chloro-1H-pyrrolo[2,3-b]pyridine 7-oxide (6 g, 20.64 mmol, 58% purity) and dimethyl sulfate (5.21 g, 41.29 mmol) in ACN (100 mL) was degassed and purged with N2 for 3 times, and then the mixture was stirred at 80° C. for 12 hr under N2 atmosphere. The mixture was used directly into next step.

### Step 3: 4-chloro-1H-pyrrolo[2,3-b]pyridine-6-carbonitrile

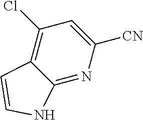 [SMILES: N#Cc1cc(Cl)c2cc[nH]c2n1]

To a solution of 4-chloro-7-methoxy-1H-pyrrolo[2,3-b]pyridin-7-ium (4.42 g, 24.07 mmol) in ACN (100 mL) was added KCN (4.70 g, 72.22 mmol) and NH₄Cl (12.88 g, 240.72 mmol) at 25° C. Then the mixture was stirred at 60° C. for 12 h. The reaction mixture was poured into saturated aqueous NaHCO₃35 mL at 25° C., and extracted with EtOAc (50 mL*3). The combined organic layers were washed with saturated brine 20 mL, dried over anhydrous Na2SO4, filtered and concentrated under reduced pressure to give a residue. The residue was purified by column chromatography (SiO2, Petroleum ether/Ethyl acetate=100/1 to 1/100) to give the product (0.8 g, crude) as a white solid. ¹H NMR (400 MHz, CHLOROFORM-d) δ=10.15 (br s, 1H), 7.42 (t, J=3.2 Hz, 1H), 7.00 (s, 1H), 6.49 (dd, J=2.0, 3.6 Hz, 1H) ppm.

### Step 4: 4-chloro-1-methyl-1H-pyrrolo[2,3-b]pyridine-6-carbonitrile

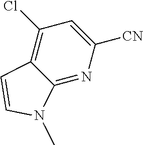

To a solution of 4-chloro-1H-pyrrolo[2,3-b]pyridine-6-carbonitrile (0.8 g, 4.50 mmol) in DMF (10 mL) was added dropwise NaH (216.21 mg, 5.41 mmol, 60% purity,) at 0° C. over 10 min. After addition, the mixture was stirred at this temperature for 10 min, and then MeI (959.09 mg, 6.76 mmol) was added dropwise at 0° C. The resulting mixture was stirred at 25° C. for 1.5 hr. The reaction mixture was poured into saturated aqueous NH₄Cl 10 mL at 25° C., and then diluted with EtOAc 20 mL and extracted with EtOAc (20 mL*3). The combined organic layers were washed with saturated brine 10 mL, dried over anhydrous Na2SO4, filtered and concentrated under reduced pressure to give a residue which was purified by column chromatography (SiO2, Petroleum ether/Ethyl acetate=100/1 to 1/100) to give the product (0.86 g, 96.64% yield) as a white solid. MS(ESI) m/e [M+1]192; ¹H NMR (400 MHz, CHLOROFORM-d) δ=7.53-7.41 (m, 2H), 6.66 (d, J=3.6 Hz, 1H), 3.94 (s, 3H) ppm.

### Step 5: methyl 4-chloro-1-methyl-1H-pyrrolo[2,3-b]pyridine-6-carboxylate

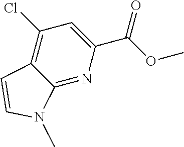

A solution of 4-chloro-1-methyl-1H-pyrrolo[2,3-b]pyridine-6-carbonitrile (0.86 g, 4.49 mmol) in HCl/MeOH (4 M, 12.29 mL) was stirred at 25° C. for 12 h. The mixture was diluted with H2O (300 mL). The reaction mixture was poured into saturated aqueous NH₄Cl 10 mL at 25° C., and then diluted with EtOAc 20 mL and extracted with EtOAc (20 mL*3). The reaction mixture was poured into saturated aqueous NH₄Cl 10 mL at 25° C., and then diluted with EtOAc 20 mL and extracted with EtOAc (20 mL*3). The combined organic layers were washed with saturated brine 10 mL, dried over anhydrous Na2SO4, filtered and concentrated under reduced pressure to give a residue which was purified by column chromatography (SiO2, Petroleum ether/Ethyl acetate=100/1 to 1/100) to give the product (0.86 g, 84.45% yield) as a white solid. MS(ESI) m/e [M+1]225. ¹H NMR (400 MHz, CHLOROFORM-d) δ=7.98 (s, 1H), 7.41 (d, J=3.6 Hz, 1H), 6.64 (d, J=3.6 Hz, 1H), 4.03 (s, 3H), 3.99 (s, 3H) ppm.

### Step 6: methyl 1-methyl-4-vinyl-1H-pyrrolo[2,3-b]pyridine-6-carboxylate

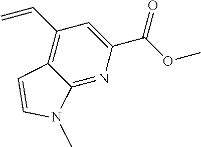 [SMILES: C=Cc1cc(C(=O)OC)nc2c1ccn2C]

A mixture of potassium; trifluoro(vinyl)boranuide (1.38 g, 10.28 mmol), methyl 4-chloro-1-methyl-1H-pyrrolo[2,3-b]pyridine-6-carboxylate (0.66 g, 2.94 mmol), cyclopentyl(diphenyl)phosphane; dichloromethane; dichloropalladium; iron (479.85 mg, 587.60 umol), K2CO3 (2.84 g, 20.57 mmol) in dioxane (30 mL) and H2O (3 mL) was degassed and purged with N2 for 3 times, and then the mixture was stirred at 100° C. for 12 hr under N2 atmosphere. The mixture was filtere and the filterate diluted with EtOAc 100 mL, extracted with EtOAc (100 mL*30 and dried over Na2SO4, filtered and concentrated to give residue which was purified by column chromatography (SiO2, Petroleum ether/Ethyl acetate=100/1 to 1/100) to give the product (0.45 g, 58.08% yield) as a white solid. MS(ESI) m/e [M+1]=217. ¹H NMR (400 MHz, CHLOROFORM-d) δ=8.05 (s, 1H), 7.40 (d, J=3.6 Hz, 1H), 7.08 (dd, J=11.2, 17.6 Hz, 1H), 6.69 (d, J=3.6 Hz, 1H), 6.18 (d, J=17.6 Hz, 1H), 5.66 (d, J=11.2 Hz, 1H), 4.04 (s, 3H), 3.99 (s, 3H) ppm.

### Step 7: methyl 4-formyl-1-methyl-1H-pyrrolo[2,3-b]pyridine-6-carboxylate

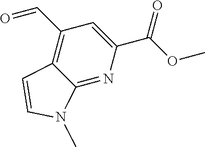 [SMILES: COC(=O)c1cc(C=O)c2ccn(C)c2n1]

To a solution of methyl 1-methyl-4-vinyl-1H-pyrrolo[2,3-b]pyridine-6-carboxylate (0.33 g, 1.25 mmol) in dioxane (10 mL) and H2O (10 mL) was added K2OsO4·2H2O (55.33 mg, 150.17 umol) and NaIO4 (749.47 mg, 3.50 mmol) at 0° C. The mixture was stirred at 0-25° C. for 2 hr. The mixture was filtered and washed with EtOAc (10 mL*3). The filterate was diluted with water 5 mL, extracted with EtOAc (10 mL*3), derived over Na2SO4, filtered and concentrated to give a residue. The residue was purified by prep-TLC (SiO2, Petroleum ether:Ethyl acetate=1:1) to give the product (0.12 g, 42.14% yield) as a white solid. MS(ESI) m/e [M+1]219. ¹H NMR (400 MHz, CHLOROFORM-d) δ=10.39 (s, 1H), 8.40 (s, 1H), 7.64 (d, J=3.6 Hz, 1H), 7.22 (d, J=3.6 Hz, 1H), 4.08 (s, 3H), 4.06 (s, 3H) ppm.

### Step 8: methyl 4-(((cyclobutylmethyl)amino)methyl)-1-methyl-1H-pyrrolo[2,3-b]pyridine-6-carboxylate

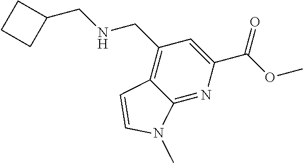 [SMILES: COC(=O)c1cc(CNCC2CCC2)c2ccn(C)c2n1]

In a mixture of methyl 4-formyl-1-methyl-1H-pyrrolo[2,3-b]pyridine-6-carboxylate (0.05 g, 229.14 umol), cyclobutylmethanamine hydrochloride (111.46 mg, 916.56 umol), AcOH (41.28 mg, 687.42 umol), TEA (92.75 mg, 916.56 umol), DCE (3 mL) was added NaBH(OAc)₃ (437.07 mg, 2.06 mmol) at 20° C. The mixture was stirred at 20° C. for 24 h. The mixture was diluted with DCM (30 ml), adjusted to pH=8 with saturated NaHCO₃ aqueous solution, extracted with DCM (50 ml*2), The combined organic layers were dried over Na2SO4, filtered and concentrated under reduced pressure to give a residue. The residue was purified by prep-TLC (EtOAc: MeOH=10:1) to give the product (0.04 g, 60.75% yield) as a white solid. MS(ESI) m/e [M+1]288. ¹H NMR (400 MHz, DMSO-d₆) δ=7.91 (s, 1H), 7.72 (d, J=3.6 Hz, 1H), 6.68 (d, J=3.6 Hz, 1H), 4.02 (s, 2H), 3.87 (d, J=13.2 Hz, 6H), 2.54 (d, J=7.2 Hz, 2H), 2.45-2.30 (m, 1H), 2.06-1.92 (m, 2H), 1.86-1.75 (m, 2H), 1.70-1.57 (m, 2H) ppm.

### Step 9: lithium 4-(((cyclobutylmethyl)amino)methyl)-1-methyl-1H-pyrrolo[2,3-b]pyridine-6-carboxylate

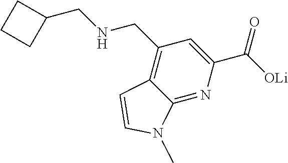

In a mixture of methyl 4-(((cyclobutylmethyl)amino)methyl)-1-methyl-1H-pyrrolo[2,3-b]pyridine-6-carboxylate (0.05 g, 174.00 umol), LiOH·H2O (9.49 mg, 226.20 umol), MeOH (1 mL), THF (1 mL), H2O (1 mL) was stirred at 20° C. for 2 h. The mixture was concentrated in vacuo to give the crude product (40 mg, crude), which was used to the next step without purification. MS(ESI) m/e [M+1]274.2

### Step 10: 4-(((cyclobutylmethyl)amino)methyl)-1-methyl-N-(3-((1s, 3s)-3-methyl-1-(4-methyl-4H-1,2,4-triazol-3-yl)cyclobutyl)phenyl)-1H-pyrrolo[2,3-b]pyridine-6-carboxamide

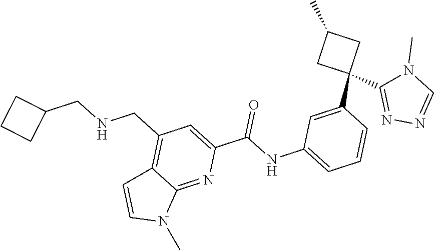 [SMILES: Cn1ccc2c(CNCC3CCC3)cc(C(=O)Nc3cccc([C@]4(c5nncn5C)C[C@@H](C)C4)c3)nc21]

To a stirred solution of lithium 4-(((cyclobutylmethyl)amino)methyl)-1-methyl-1H-pyrrolo[2,3-b]pyridine-6-carboxylate (40 mg, 107.43 μmol) and 3-((1s, 3s)-3-methyl-1-(4-methyl-4H-1,2,4-triazol-3-yl)cyclobutyl)aniline (26.03 mg, 107.43 mol) in pyridine (2 mL) was added T3P (205.09 mg, 322.28 mol, 50% purity) at 25° C. The resulting mixture was stirred for 3 h at 25° C. The reaction mixture was quenched by addition H2O 2 mL at 20° C., and then extracted with EtOAc 15 mL (5 mL*3). The combined organic layers were washed with brine 10 mL (5 mL*2), dried over Na2SO4, filtered and concentrated under reduced pressure to give a residue. The crude was purified by prep-HPLC(column: Waters Xbridge BEH C18 100*30 mm*10 um; mobile phase: [water (NH₄HCO3)-ACN]; B %: 35%-65%, 8 min) to give the product (15 mg, 28.06% yield). MS(ESI) m/e [M+1]498. ¹H NMR (400 MHz, DMSO-d₆) δ=10.47 (s, 1H), 8.29 (s, 1H), 7.96 (s, 1H), 7.92 (s, 1H), 7.84 (br d, J=8.0 Hz, 1H), 7.71 (d, J=3.6 Hz, 1H), 7.37 (t, J=8.0 Hz, 1H), 7.02 (br d, J=8.0 Hz, 1H), 6.70 (d, J=3.6 Hz, 1H), 4.04 (s, 2H), 4.00 (s, 3H), 3.20 (s, 3H), 2.85 (br d, J=3.6 Hz, 2H), 2.55 (br d, J=6.4 Hz, 6H), 2.06-1.93 (m, 2H), 1.91-1.74 (m, 2H), 1.72-1.57 (m, 2H), 1.09 (br d, J=5.5 Hz, 3H) ppm.

### Compound B46: 7-{[(cyclobutylmethyl)amino]methyl}-3-fluoro-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide

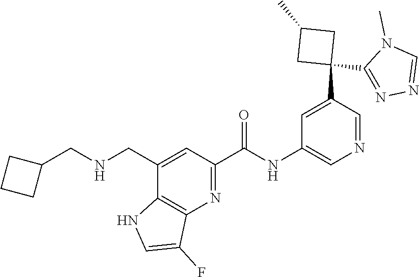 [SMILES: Cn1cnnc1[C@]1(c2cncc(NC(=O)c3cc(CNCC4CCC4)c4[nH]cc(F)c4n3)c2)C[C@H](C)C1]

### Step 1: 7-chloro-3-fluoro-1H-pyrrolo[3,2-b]pyridine-5-carbonitrile

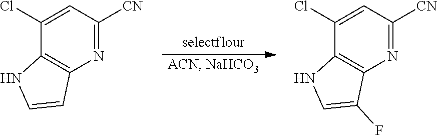

To a stirred solution of 7-chloro-IH-pyrrolo[3,2-b]pyridine-5-carbonitrile (5 g, 26.747 mmol, 1 equiv, 95.0%) and Selectfluor (11.97 g, 32.099 mmol, 1.20 equiv, 95%) in ACN (150 mL, 100%) was added NaHCO₃ (4.73 g, 53.494 mmol, 2 equiv, 95%) at room temperature. The resulting mixture was stirred for 1 h at 60° C. under nitrogen atmosphere. The mixture was allowed to cool down to room temperature. The resulting mixture was concentrated under reduced pressure. The residue was purified by silica gel column chromatography, eluted with PE/EA (1:1) to afford 7-chloro-3-fluoro-1H-pyrrolo[3,2-b]pyridine-5-carbonitrile (1.7 g, 25.97%). MS: M/e 194 (M+H)⁺.

### Step 2: 7-ethenyl-3-fluoro-IH-pyrrolo[3,2-b]pyridine-5-carbonitrile

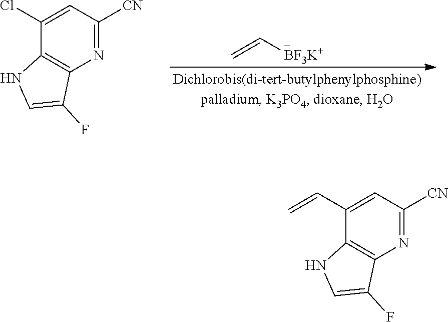

To a stirred solution of 7-chloro-3-fluoro-1H-pyrrolo[3,2-b]pyridine-5-carbonitrile (1.7 g, 6.945 mmol, 1 equiv, 79.9%) and potassium ethenyltrifluoroboranuide (1.47 g, 10.418 mmol, 1.5 equiv, 95%) in dioxane (80 mL, 100%) and H₂O (8 mL, 100%) were added K₃PO₄ (3.10 g, 13.890 mmol, 2 equiv, 95%) and di-tert-butyl( )phenyl-lambda5-phosphane (0.46 g, 0.695 mmol, 0.1 equiv, 95%) at room temperature. The resulting mixture was stirred for 4 hrs at 100° C. under nitrogen atmosphere. The mixture was allowed to cool down to room temperature. The resulting mixture was concentrated under reduced pressure. The residue was purified by silica gel column chromatography, eluted with PE/EA (3:1) to afford 7-ethenyl-3-fluoro-1H-pyrrolo[3,2-b]pyridine-5-carbonitrile (500 mg, 30.81%). MS: M/e 188 (M+H)⁺.

### Step 3: 3-fluoro-7-formyl-1H-pyrrolo[3,2-b]pyridine-5-carbonitrile

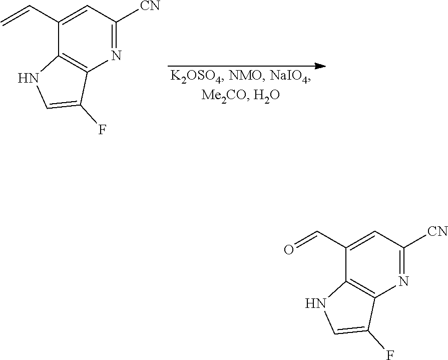

To a stirred solution of 7-ethenyl-3-fluoro-1H-pyrrolo[3,2-b]pyridine-5-carbonitrile (200 mg, 0.856 mmol, 1 equiv, 80.1%) in acetone (12 mL, 100%) and H₂O (2.4 mL, 100%) were added NMO (158.31 mg, 1.284 mmol, 1.5 equiv, 95%) and K₂OsO₄·2H₂O (33.19 mg, 0.086 mmol, 0.1 equiv, 95%) at room temperature. The resulting mixture was stirred for 2 hrs at room temperature. To the above mixture was added NaIO₄ (578.09 mg, 2.568 mmol, 3 equiv, 95%) at room temperature. The resulting mixture was stirred for additional overnight at room temperature. The resulting mixture was diluted with MeOH (10 mL). The resulting mixture was filtered, the filter cake was washed with MeOH (2×2 mL). The filtrate was concentrated under reduced pressure. The residue was purified by silica gel column chromatography, eluted with PE/EA (1:1) to afford 3-fluoro-7-formyl-1H-pyrrolo[3,2-b]pyridine-5-carbonitrile (100 mg, 29.53%). MS: M/e 190 (M+H)⁺.

### Step 4: 7-(((cyclobutylmethyl)amino)methyl)-3-fluoro-1H-pyrrolo[3,2-b]pyridine-5-carbonitrile

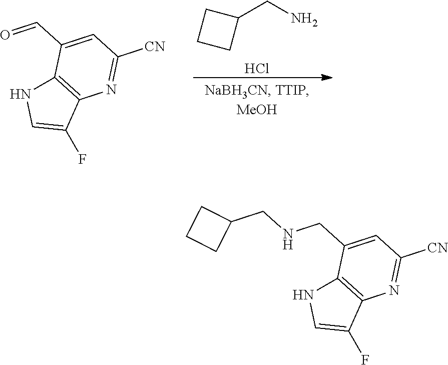 [SMILES: N#Cc1cc(C=O)c2[nH]cc(F)c2n1 ; NCC1CCC1 ; Cl ; N#Cc1cc(CNCC2CCC2)c2[nH]cc(F)c2n1]

To a stirred solution of 3-fluoro-7-formyl-1H-pyrrolo[3,2-b]pyridine-5-carbonitrile (100 mg, 0.253 mmol, 1 equiv, 47.8%) and 1-cyclobutylmethanamine hydrochloride (64.70 mg, 0.506 mmol, 2 equiv, 95%) in MeOH (5 mL, 100%) were added tetrakis(propan-2-yloxy)titanium (302.42 mg, 1.012 mmol, 4 equiv, 95%) at room temperature. The resulting mixture was stirred for 1 h at room temperature. To the above mixture was added NaBH₃CN (33.43 mg, 0.506 mmol, 2 equiv, 95%) at room temperature. The resulting mixture was stirred for additional 1 h at room temperature. The resulting mixture was concentrated under reduced pressure. The residue was purified by silica gel column chromatography, eluted with CH₂Cl₂/MeOH (9:1) to afford 7-(((cyclobutylmethyl)amino)methyl)-3-fluoro-1H-pyrrolo[3,2-b]pyridine-5-carbonitrile (80 mg, 93.51%). MS: M/e 259 (M+H)⁺.

### Step 5: 7-(((cyclobutylmethyl)amino)methyl)-3-fluoro-1H-pyrrolo[3,2-b]pyridine-5-carboxylic acid

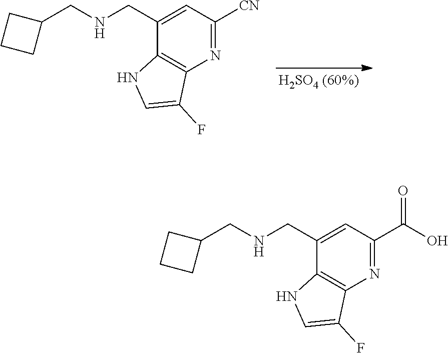 [SMILES: N#Cc1cc(CNCC2CCC2)c2[nH]cc(F)c2n1 ; O=C(O)c1cc(CNCC2CCC2)c2[nH]cc(F)c2n1]

A solution of 7-(((cyclobutylmethyl)amino)methyl)-3-fluoro-1H-pyrrolo[3,2-b]pyridine-5-carbonitrile (70 mg, 0.207 mmol, 1 equiv, 76.3%) in H₂SO₄ (4 mL, 45.029 mmol, 217.77 equiv, 60%) was stirred for 1 h at 100° C. The mixture was allowed to cool down to 0° C. The mixture was neutralized to pH=7 with NaOH. The resulting mixture was concentrated under vacuum. The resulting mixture was diluted with MeOH (10 mL). The resulting mixture was filtered, the filter cake was washed with MeOH (2×2 mL). The filtrate was concentrated under reduced pressure. The residue was purified by reversed-phase flash chromatography with the following conditions: column, C18 silica gel; mobile phase, MeCN in Water (0.1% TFA), 0% to 20% gradient in 10 min; detector, UV 254 nm. This resulted in 7-(((cyclobutylmethyl)amino)methyl)-3-fluoro-1H-pyrrolo[3,2-b]pyridine-5-carboxylic acid (50 mg, 86.33%). MS: M/e 278 (M+H)⁺.

### Step 6: 7-{[(cyclobutylmethyl)amino]methyl}-3-fluoro-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide

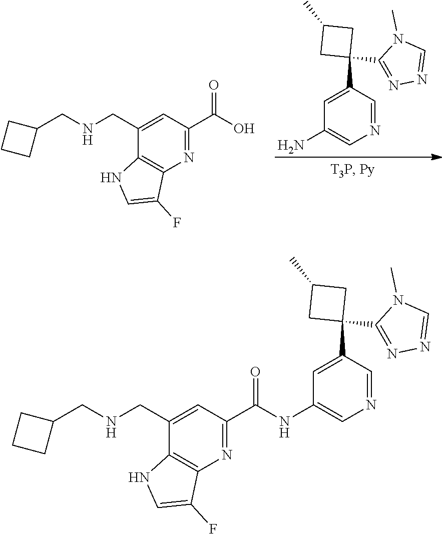 [SMILES: O=C(O)c1cc(CNCC2CCC2)c2[nH]cc(F)c2n1 ; Cn1cnnc1[C@]1(c2cncc(N)c2)C[C@H](C)C1 ; Cn1cnnc1[C@]1(c2cncc(NC(=O)c3cc(CNCC4CCC4)c4[nH]cc(F)c4n3)c2)C[C@H](C)C1]

To a stirred solution of 7-(((cyclobutylmethyl)amino)methyl)-3-fluoro-1H-pyrrolo[3,2-b]pyridine-5-carboxylic acid (50 mg, 0.179 mmol, 1 equiv, 99.0%) and 5-[(1r, 3s)-3-methyl-1-(4-methyl-1,2,4-triazol-3-yl)cyclobutyl]pyridin-3-amine (45.72 mg, 0.179 mmol, 1 equiv, 95%) in Pyridine (4 mL, 100%) was added T₃P (179.36 mg, 0.537 mmol, 3 equiv, 95%) at room temperature. The resulting mixture was stirred for 1 h at room temperature. The resulting mixture was concentrated under vacuum. The crude product was purified by Chiral-Prep-HPLC with the following conditions (2 #SHIMADZU (HPLC-01)): Column, YMC-Actus Triart C18 ExRS, 30*150 mm, 5 μm; mobile phase, 10 mmol NH4HCO₃+0.05% NH₃H₂O and ACN (36% ACN up to 66% in 8 min); This resulted in 7-{[(cyclobutylmethyl)amino]methyl}-3-fluoro-N-{5-[(1r, 3s)-3-methyl-1-(4-methyl-1,2,4-triazol-3-yl)cyclobutyl]pyridin-3-yl}-1H-pyrrolo[3,2-b]pyridine-5-carboxamide (31.4 mg, 34.75%). MS: M/e 503 (M+H)⁺. ¹H NMR (300 MHz, DMSO-d₆) δ 10.74 (s, 1H), 9.11 (d, J=2.2 Hz, 1H), 8.37-8.23 (m, 3H), 8.07 (s, 1H), 7.84 (d, J=2.4 Hz, 1H), 4.07 (s, 2H), 3.23 (s, 3H), 2.94-2.83 (m, 2H), 2.63-2.52 (m, 5H), 2.50-2.34 (m, 1H), 2.08-1.93 (m, 2H), 1.92-1.71 (m, 2H), 1.75-1.55 (m, 2H), 1.10 (d, J=5.5 Hz, 3H).

### Compound B47: 7-((((S)-sec-butyl)amino)methyl)-N-(5-((1s,3R)-3-methyl-1-(4-methyl-4H-1,2,4-triazol-3-yl)cyclobutyl)pyridin-3-yl)-1H-pyrrolo[3,2-b]pyridine-5-carboxamide

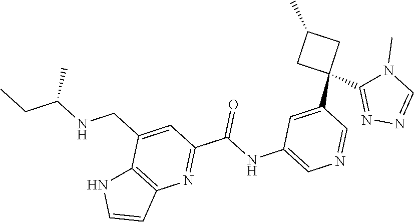 [SMILES: CC[C@H](C)NCc1cc(C(=O)Nc2cncc([C@]3(c4nncn4C)C[C@@H](C)C3)c2)nc2cc[nH]c12]

### Step A: (S)-7-((sec-butylamino)methyl)-1H-pyrrolo[3,2-b]pyridine-5-carbonitrile

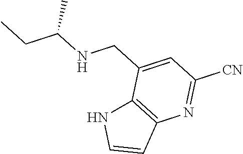 [SMILES: CC[C@H](C)NCc1cc(C#N)nc2cc[nH]c12]

A solution of 7-formyl-1H-pyrrolo[3,2-b]pyridine-5-carbonitrile (100 mg, 0.58 mmol), (S)-butan-2-amine hydrochloride (120 mg, 1.1 mmol) and TEA (111 mg, 1.1 mmol) in DCM (3 mL) was stirred at r.t for 20 mins. Then it was cooled under ice bath, NaBH(OAc)₃ (233 mg, 1.1 mmol) was added and it was stirred at r.t overnight. More of NaBH(OAc)₃ (116 mg, 0.6 mmol) was added and it continued to stir at r.t for 5 hrs. The reaction mixture was added with water (5 mL), basified with NaHCO₃ solution and extracted with DCM (10 mL). The organic layer was dried, concentrated and purified by CombiFlash (DCM:MeOH=5%) to get the product (35 mg, 44%). MS: M/e 229 (M+1)⁺

### Step B: (S)-7-((sec-butylamino)methyl)-1H-pyrrolo[3,2-b]pyridine-5-carboxylic acid

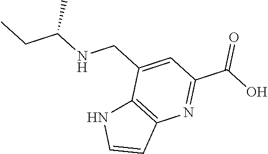 [SMILES: CC[C@H](C)NCc1cc(C(=O)O)nc2cc[nH]c12]

A solution of (S)-7-((sec-butylamino)methyl)-1H-pyrrolo[3,2-b]pyridine-5-carbonitrile (35 mg, 0.15 mmol) in concentrated H₂SO₄ (1 mL) was heated at 100° C. overnight. The reaction mixture was basified with NH₃·MeOH (7 M) to pH=8-9. The precipitated solid was filtered. The filtrate was evaporated, dissolved in water (3 mL) and lyophilized to get the product (35 mg, crude). MS: M/e 248 (M+1)⁺

### Step C: 7-((((S)-sec-butyl)amino)methyl)-N-(5-((1s,3R)-3-methyl-1-(4-methyl-4H-1,2,4-triazol-3-yl)cyclobutyl)pyridin-3-yl)-1H-pyrrolo[3,2-b]pyridine-5-carboxamide

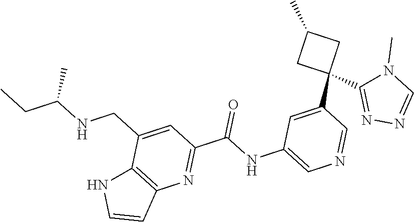 [SMILES: CC[C@H](C)NCc1cc(C(=O)Nc2cncc([C@]3(c4nncn4C)C[C@@H](C)C3)c2)nc2cc[nH]c12]

To a solution of (S)-7-((sec-butylamino)methyl)-1H-pyrrolo[3,2-b]pyridine-5-carboxylic acid (35 mg, 0.14 mmol) and 5-((1s, 3s)-3-methyl-1-(4-methyl-4H-1,2,4-triazol-3-yl)cyclobutyl)pyridin-3-amine (34 mg, 0.14 mmol) in pyridine (3 mL) was added with T₃P (267 mg, 0.42 mmol). The reaction mixture was stirred at r.t overnight, then added with water (3 mL) and extracted with DCM (10 mL). The organic layer was dried, concentrated and purified by prep. HPLC (Mobile Phase A: 0.1% FA-H2O, Mobile Phase B: 0.1% FA-ACN) to get the product (31 mg, 47%). ¹H NMR (400 MHz, CD₃OD) δ 9.02 (s, 1H), 8.49 (s, 1H), 8.36 (s, 1H), 8.32 (s, 1H), 8.16 (s, 1H), 7.82 (d, J=4.0 Hz, 1H), 6.84 (d, J=4.0 Hz, 1H), 4.62-4.54 (m, 2H), 3.36 (s, 3H), 3.25-3.18 (m, 1H), 3.01-2.96 (m, 2H), 2.75-2.63 (m, 3H), 1.98-1.92 (m, 1H), 1.67-1.56 (m, 1H), 1.40 (d, J=8.0 Hz, 3H), 1.17 (d, J=4.0 Hz, 3H), 1.04 (t, J=8.0 Hz, 3H) ppm. MS: M/e 473 (M+1)⁺

### Compound B48: 3-fluoro-7-((isobutylamino)methyl)-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

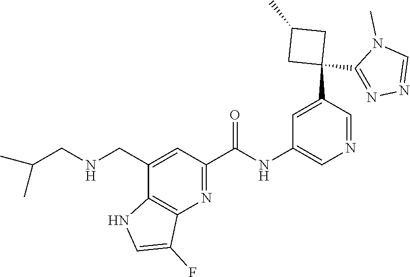 [SMILES: CC(C)CNCc1cc(C(=O)Nc2cncc([C@]3(c4nncn4C)C[C@@H](C)C3)c2)nc2c(F)c[nH]c12]

### Step 1: 3-fluoro-7-((isobutylamino)methyl)-1H-pyrrolo[3,2-b]pyridine-5-carbonitrile

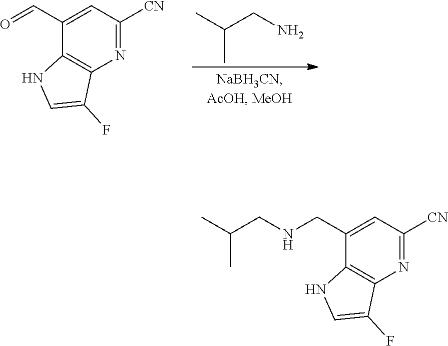

To a stirred solution of 3-fluoro-7-formyl-1H-pyrrolo[3,2-b]pyridine-5-carbonitrile (60 mg, 0.172 mmol, 1 equiv, 54.2%) and isobutylamine (26.47 mg, 0.344 mmol, 2 equiv, 95%) in MeOH (3 mL, 100%) were added AcOH (21.74 mg, 0.344 mmol, 2 equiv, 95%) at room temperature. The resulting mixture was stirred for 1 h at room temperature. To the above mixture was added NaBH₃CN (22.75 mg, 0.344 mmol, 2 equiv, 95%) at room temperature. The resulting mixture was stirred for additional 1 h at room temperature. The resulting mixture was concentrated under reduced pressure. The residue was purified by silica gel column chromatography, eluted with CH₂Cl₂/MeOH (9:1) to afford 3-fluoro-7-((isobutylamino)methyl)-1H-pyrrolo[3,2-b]pyridine-5-carbonitrile (45 mg, 95.65%). MS: M/e 247 (M+H)⁺.

### Step 2: 3-fluoro-7-((isobutylamino)methyl)-1H-pyrrolo[3,2-b]pyridine-5-carboxylic acid

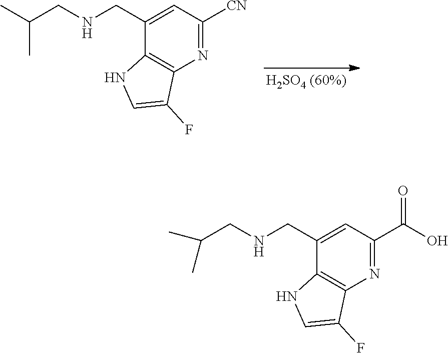 [SMILES: CC(C)CNCc1cc(C#N)nc2c(F)c[nH]c12 ; CC(C)CNCc1cc(C(=O)O)nc2c(F)c[nH]c12]

A solution of 3-fluoro-7-((isobutylamino)methyl)-1H-pyrrolo[3,2-b]pyridine-5-carbonitrile (45 mg, 0.164 mmol, 1 equiv, 90%) in H₂SO₄ (2 mL, 22.515 mmol, 136.92 equiv, 60%) was stirred for 1 h at 100° C. The mixture was allowed to cool down to 0° C. The mixture was neutralized to pH=7 with NaOH. The resulting mixture was diluted with MeOH (5 mL). The resulting mixture was filtered, the filter cake was washed with MeOH (2×2 mL). The filtrate was concentrated under reduced pressure. The residue was purified by reversed-phase flash chromatography with the following conditions: column, C18 silica gel; mobile phase, MeCN in Water (0.1% TFA), 0% to 10% gradient in 10 min; detector, UV 254 nm. This resulted in 3-fluoro-7-((isobutylamino)methyl)-1H-pyrrolo[3,2-b]pyridine-5-carboxylic acid (40 mg, 87.11%). MS: M/e 266 (M+H)⁺.

### Step 3: 3-fluoro-7-((isobutylamino)methyl)-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

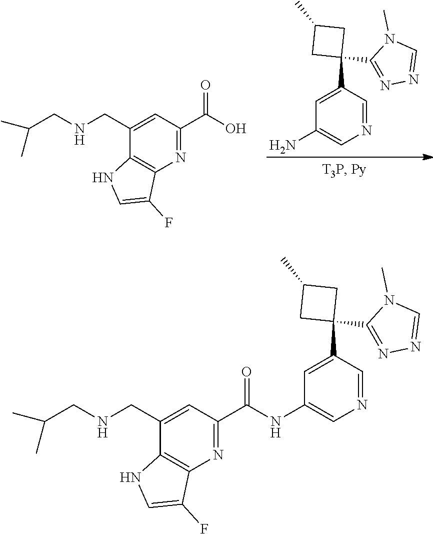 [SMILES: CC(C)CNCc1cc(C(=O)O)nc2c(F)c[nH]c12 ; Cn1cnnc1[C@]1(c2cncc(N)c2)C[C@H](C)C1 ; CC(C)CNCc1cc(C(=O)Nc2cncc([C@]3(c4nncn4C)C[C@@H](C)C3)c2)nc2c(F)c[nH]c12]

To a stirred solution of 3-fluoro-7-((isobutylamino)methyl)-1H-pyrrolo[3,2-b]pyridine-5-carboxylic acid (40 mg, 0.143 mmol, 1 equiv, 95%) and 5-[(1r, 3s)-3-methyl-1-(4-methyl-1,2,4-triazol-3-yl)cyclobutyl]pyridin-3-amine (36.69 mg, 0.143 mmol, 1 equiv, 95%) in Pyridine (2 mL, 100%) was added T₃P (143.93 mg, 0.429 mmol, 3 equiv, 95%) at room temperature. The resulting mixture was stirred for 1 h at room temperature. The resulting mixture was concentrated under vacuum. The crude product was purified by Chiral-Prep-HPLC with the following conditions (2 #SHIMADZU (HPLC-01)): Column, YMC-Actus Triart C18 ExRS, 30*150 mm, 5 μm; mobile phase, 10 mmol NH₄HCO₃+0.05% NH₃H₂O and ACN (31% ACN up to 61% in 8 min); This resulted in 3-fluoro-7-((isobutylamino)methyl)-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide (15.0 mg, 20.85%). MS: M/e 491 (M+H). ¹H NMR (300 MHz, DMSO-d₆) δ 10.74 (s, 1H), 9.11 (d, J=2.2 Hz, 1H), 8.34 (s, 1H), 8.32-8.29 (m, 1H), 8.26 (d, J=2.1 Hz, 1H), 8.09 (s, 1H), 7.83 (d, J=2.4 Hz, 1H), 4.08 (s, 2H), 3.24 (s, 3H), 2.89 (d, J=4.7 Hz, 2H), 2.59 (d, J=7.6 Hz, 3H), 2.35 (d, J=6.7 Hz, 2H), 1.82-1.63 (m, 1H), 1.14-1.07 (m, 3H), 0.89 (d, J=6.6 Hz, 6H).

### Compound B49: 3-fluoro-N-(5-((1s, 3s)-3-methyl-1-(4-methyl-4H-1,2,4-triazol-3-yl)cyclobutyl)pyridin-3-yl)-7-(((1-methylcyclobutyl)amino)methyl)-1H-pyrrolo[3,2-b]pyridine-5-carboxamide

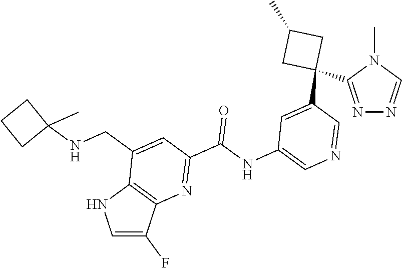 [SMILES: Cn1cnnc1[C@]1(c2cncc(NC(=O)c3cc(CNC4(C)CCC4)c4[nH]cc(F)c4n3)c2)C[C@H](C)C1]

### Step 1: 3-fluoro-7-(((1-methylcyclobutyl)amino)methyl)-1H-pyrrolo[3,2-b]pyridine-5-carbonitrile

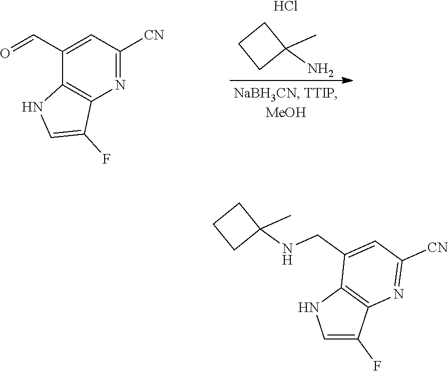

To a stirred solution of 3-fluoro-7-formyl-1H-pyrrolo[3,2-b]pyridine-5-carbonitrile (90 mg, 0.258 mmol, 1 equiv, 54.2%) and 1-methylcyclobutan-1-amine hydrochloride (66.03 mg, 0.516 mmol, 2 equiv, 95%) in MeOH (4 mL, 100%) were added tetrakis(propan-2-yloxy)titanium (308.62 mg, 1.032 mmol, 4 equiv, 95%) at room temperature. The resulting mixture was stirred for 1 h at 60° C. To the above mixture was added NaBH₃CN (34.12 mg, 0.516 mmol, 2 equiv, 95%) at room temperature. The resulting mixture was stirred for additional 1 h at room temperature. The resulting mixture was concentrated under reduced pressure. The residue was purified by silica gel column chromatography, eluted with CH₂Cl₂/MeOH (9:1) to afford 3-fluoro-7-(((1-methylcyclobutyl)amino)methyl)-1H-pyrrolo[3,2-b]pyridine-5-carbonitrile (60 mg, 72.06%). MS: M/e 259 (M+H)⁺.

### Step 2: 3-fluoro-7-(((1-methylcyclobutyl)amino)methyl)-1H-pyrrolo[3,2-b]pyridine-5-carboxylic acid

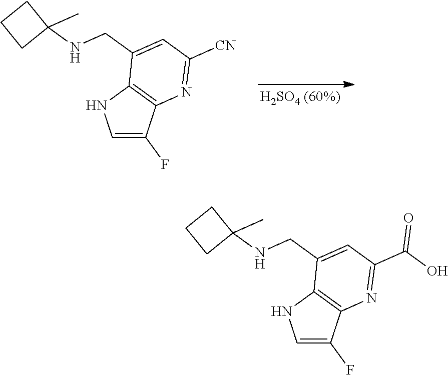 [SMILES: CC1(NCc2cc(C#N)nc3c(F)c[nH]c23)CCC1 ; CC1(NCc2cc(C(=O)O)nc3c(F)c[nH]c23)CCC1]

A solution of 3-fluoro-7-(((1-methylcyclobutyl)amino)methyl)-1H-pyrrolo[3,2-b]pyridine-5-carbonitrile (60 mg, 0.186 mmol, 1 equiv, 80.0%) in H₂SO₄ (2 mL, 22.515 mmol, 121.16 equiv, 60%) was stirred for 1 h at 100° C. The mixture was allowed to cool down to 0° C. The mixture was neutralized to pH=7 with NaOH. The resulting mixture was concentrated under vacuum. The resulting mixture was diluted with MeOH (5 mL). The resulting mixture was filtered, the filter cake was washed with MeOH (2×2 mL). The filtrate was concentrated under reduced pressure. The residue was purified by reversed-phase flash chromatography with the following conditions: column, C18 silica gel; mobile phase, MeCN in Water (0.1% TFA), 0% to 10% gradient in 10 min; This resulted in 3-fluoro-7-(((1-methylcyclobutyl)amino)methyl)-1H-pyrrolo[3,2-b]pyridine-5-carboxylic acid (50 mg, 92.18%). MS: M/e 278 (M+H)⁺.

### Step 3: 3-fluoro-N-(5-((1s, 3s)-3-methyl-1-(4-methyl-4H-1,2,4-triazol-3-yl)cyclobutyl)pyridin-3-yl)-7-(((1-methylcyclobutyl)amino)methyl)-1H-pyrrolo[3,2-b]pyridine-5-carboxamide

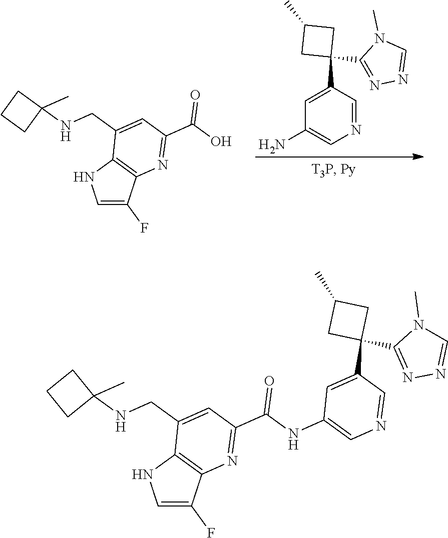 [SMILES: CC1(NCc2cc(C(=O)O)nc3c(F)c[nH]c23)CCC1 ; Cn1cnnc1[C@]1(c2cncc(N)c2)C[C@H](C)C1 ; Cn1cnnc1[C@]1(c2cncc(NC(=O)c3cc(CNC4(C)CCC4)c4[nH]cc(F)c4n3)c2)C[C@H](C)C1]

To a stirred solution of 3-fluoro-7-(((1-methylcyclobutyl)amino)methyl)-1H-pyrrolo[3,2-b]pyridine-5-carboxylic acid (50 mg, 0.171 mmol, 1 equiv, 95.0%) and 5-[(1r, 3s)-3-methyl-1-(4-methyl-1,2,4-triazol-3-yl)cyclobutyl]pyridin-3-amine (43.87 mg, 0.171 mmol, 1 equiv, 95.0%) in Pyridine (2 mL, 100%) was added T₃P (172.11 mg, 0.513 mmol, 3 equiv, 95%) at room temperature. The resulting mixture was stirred for 1 h at room temperature. The resulting mixture was concentrated under vacuum. The crude product was purified by Chiral-Prep-HPLC with the following conditions (2 #SHIMADZU (HPLC-01)): Column, YMC-Actus Triart C18 ExRS, 30*150 mm, 5 μm; mobile phase, 10 mmol NH₄HCO₃+0.05% NH₃H₂O and ACN (25% ACN up to 55% in 8 min); This resulted in 3-fluoro-N-(5-((1s, 3s)-3-methyl-1-(4-methyl-4H-1,2,4-triazol-3-yl)cyclobutyl)pyridin-3-yl)-7-(((1-methylcyclobutyl)amino)methyl)-1H-pyrrolo[3,2-b]pyridine-5-carboxamide (33.0 mg, 38.29%). MS: M/e 503 (M+H)⁺.

¹H NMR (300 MHz, DMSO-d₆) δ 10.74 (s, 1H), 9.11 (d, J=2.2 Hz, 1H), 8.35 (s, 1H), 8.31 (t, J=2.2 Hz, 1H), 8.25 (d, J=2.1 Hz, 1H), 8.15 (s, 1H), 7.83 (d, J=2.4 Hz, 1H), 4.02 (s, 2H), 3.24 (s, 3H), 2.89 (q, J=6.2 Hz, 2H), 2.59 (d, J=7.6 Hz, 3H), 2.11-1.95 (m, 2H), 1.83-1.61 (m, 4H), 1.27 (s, 3H), 1.11 (d, J=5.4 Hz, 3H).

### Compound B50: N-(5-((1s, 3s)-3-methyl-1-(4-methyl-4H-1,2,4-triazol-3-yl)cyclobutyl)pyridin-3-yl)-7-(((1-methylcyclobutyl)amino)methyl)-1H-pyrrolo[3,2-b]pyridine-5-carboxamide

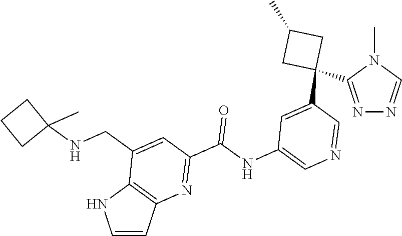 [SMILES: Cn1cnnc1[C@]1(c2cncc(NC(=O)c3cc(CNC4(C)CCC4)c4[nH]ccc4n3)c2)C[C@H](C)C1]

### Step A: 7-(((1-methylcyclobutyl)amino)methyl)-1H-pyrrolo[3,2-b]pyridine-5-carbonitrile

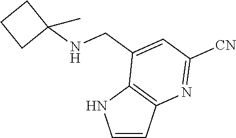 [SMILES: CC1(NCc2cc(C#N)nc3cc[nH]c23)CCC1]

A solution of 7-formyl-1H-pyrrolo[3,2-b]pyridine-5-carbonitrile (100 mg, 0.58 mmol), 1-methylcyclobutan-1-amine hydrochloride (141 mg, 1.1 mmol) and TEA (111 mg, 1.1 mmol) in DCM (3 mL) was stirred at r.t for 20 mins. Then it was cooled under ice bath, NaBH(OAc)₃ (233 mg, 1.1 mmol) was added and it was stirred at r.t overnight. More of NaBH(OAc)₃ (116 mg, 0.6 mmol) was added and it continued to stir at r.t for 5 hrs. The reaction mixture was added with water (5 mL), basified with NaHCO₃ solution and extracted with DCM (10 mL). The organic layer was dried, concentrated and purified by CombiFlash (DCM:MeOH=5%) to get the product (100 mg, 71%). MS: M/e 241 (M+1)⁺

### Step B: 7-(((1-methylcyclobutyl)amino)methyl)-1H-pyrrolo[3,2-b]pyridine-5-carboxylic acid

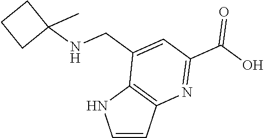 [SMILES: CC1(NCc2cc(C(=O)O)nc3cc[nH]c23)CCC1]

A solution of 7-(((1-methylcyclobutyl)amino)methyl)-1H-pyrrolo[3,2-b]pyridine-5-carbonitrile (100 mg, 0.41 mmol) in concentrated H₂SO₄ (2 mL) was heated at 100° C. for 4 hrs. The reaction mixture was basified with NH₃·MeOH (7 M) to pH=8-9. The precipitated solid was filtered. The filtrate was evaporated, dissolved in water (5 mL) and lyophilized to get the product (90 mg, crude). MS: M/e 260 (M+1)⁺

### Step C: N-(5-((1s, 3s)-3-methyl-1-(4-methyl-4H-1,2,4-triazol-3-yl)cyclobutyl) pyridin-3-yl)-7-(((1-methylcyclobutyl)amino)methyl)-1H-pyrrolo[3,2-b]pyridine-5-carboxamide

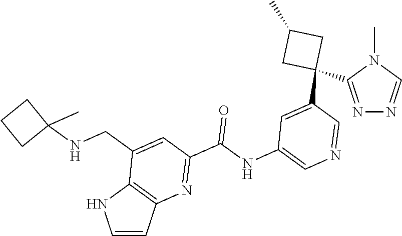 [SMILES: Cn1cnnc1[C@]1(c2cncc(NC(=O)c3cc(CNC4(C)CCC4)c4[nH]ccc4n3)c2)C[C@H](C)C1]

To a solution of 7-(((1-methylcyclobutyl)amino)methyl)-1H-pyrrolo[3,2-b]pyridine-5-carboxylic acid (90 mg, 0.34 mmol) and 5-((1s, 3s)-3-methyl-1-(4-methyl-4H-1,2,4-triazol-3-yl)cyclobutyl)pyridin-3-amine (84 mg, 0.34 mmol) in pyridine (4 mL) was added with T3P (636 mg, 1 mmol). The reaction mixture was stirred at r.t for 3 hrs, then added with water (3 mL) and extracted with DCM (10 mL). The organic layer was dried, concentrated and purified by prep. HPLC (Mobile Phase A: 0.1% FA-H2O, Mobile Phase B: 0.1% FA-ACN) to get the product (80 mg, 48%). ¹H NMR (400 MHz, CD₃OD) δ 9.02 (d, J=4.0 Hz, 1H), 8.39 (t, J=4.0 Hz, 1H), 8.36 (s, 1H), 8.31 (d, J=4.0 Hz, 1H), 8.13 (s, 1H), 7.80 (d, J=4.0 Hz, 1H), 6.81 (d, J=4.0 Hz, 1H), 4.41 (s, 2H), 3.35 (s, 3H), 3.00-2.96 (m, 2H), 2.74-2.63 (m, 3H), 2.46-2.38 (m, 2H), 2.11-2.04 (m, 2H), 2.00-1.92 (m, 2H), 1.63 (s, 3H), 1.16 (d, J=4.0 Hz, 3H) ppm. MS: M/e 485 (M+1)⁺

### Compound B51: N-(5-((1s, 3s)-3-methyl-1-(4-methyl-4H-1,2,4-triazol-3-yl)cyclobutyl)pyridin-3-yl)-4-(((1-methylcyclopropyl)amino)methyl)thieno[2,3-b]pyridine-6-carboxamide

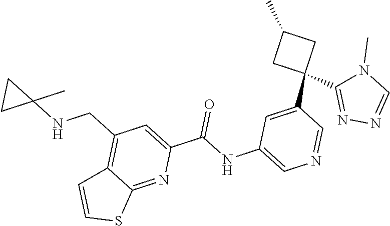 [SMILES: Cn1cnnc1[C@]1(c2cncc(NC(=O)c3cc(CNC4(C)CC4)c4ccsc4n3)c2)C[C@H](C)C1]

### Step 1: methyl 4-chlorothieno[2,3-b]pyridine-6-carboxylate

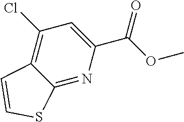

To a solution of 4,6-dichlorothieno[2,3-b]pyridine (2 g, 9.80 mmol), TEA (2.98 g, 29.40 mmol) in MeOH (40 mL) was added Pd(dppf)Cl2 (717.09 mg, 980.03 mol) one portion. The suspension was degassed under vacuum and purged with CO several times. The mixture was stirred under CO (15 psi) at 60° C. for 12 hours. The mixture was filtered and concentrated, which was purified by column chromatography (SiO2, Petroleum ether/Ethyl acetate=20:1 to 5:1) to give the product (1.34 g, 57.70% yield,) as a white solid. MS(ESI) m/e [M+1]228. ¹H NMR (400 MHz, DMSO-d₆) δ=8.32 (d, J=6.0 Hz, 1H), 8.14 (s, 1H), 7.62 (d, J=6.0 Hz, 1H), 3.94 (s, 3H)

### Step 2: methyl 4-vinylthieno[2,3-b]pyridine-6-carboxylate

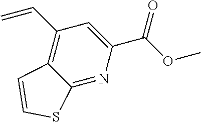

To a solution of methyl 4-chlorothieno[2,3-b]pyridine-6-carboxylate (1.34 g, 5.89 mmol) in dioxane (15 mL) and H₂O (3 mL) was added potassium; trifluoro(vinyl)boranuide (1.18 g, 8.83 mmol), K₂CO₃ (1.63 g, 11.77 mmol,) and Pd(PPh3)₄ (1.02 g, 882.87 mol). The reaction mixture was quenched by addition H₂O 5 mL at 20° C., and then extracted with DCM 15 mL (5 mL*3). The combined organic layers were washed with brine 10 mL (5 mL*2), dried over Na2SO4, filtered and concentrated under reduced pressure to give a residue, which was purified by Prep-TLC (SiO2, Petroleum ether: Ethyl acetate=3:1) to give the product (700 mg, 53.47% yield) as a white solid. MS(ESI) m/e [M+1]220. ¹H NMR (400 MHz, DMSO-d₆) δ=8.22 (s, 1H), 8.18 (d, J=6.1 Hz, 1H), 7.84 (d, J=6.0 Hz, 1H), 7.37 (dd, J=11.1, 17.6 Hz, 1H), 6.34 (d, J=17.5 Hz, 1H), 5.78 (d, J=11.0 Hz, 1H), 3.93 (s, 3H)

### Step 3: methyl 4-formylthieno[2,3-b]pyridine-6-carboxylate

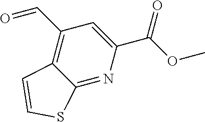

To a solution of methyl 4-vinylthieno[2,3-b]pyridine-6-carboxylate (0.8 g, 3.65 mmol) in dioxane (4 mL) and H2O (0.8 mL) was added K2OsO4·2H2O (134.44 mg, 364.86 mol). NaIO4 (3.90 g, 18.24 mmol) was added to the mixture at 0° C. The mixture was stirred at 0-20° C. for 2 hr. The mixture was filtered and extracted with EtOAc (15 mL*2), washed with brine (10 mL), dried over Na2SO4, filtered and concentrated in vacuum to get a residue, which was triturated with MTBE (5 mL) to get the product (0.6 g, 74.33% yield) as a white solid. MS(ESI) m/e [M+1]223

### Step 4: methyl 4-(((1-methylcyclopropyl)amino)methyl)thieno[2,3-b]pyridine-6-carboxylate

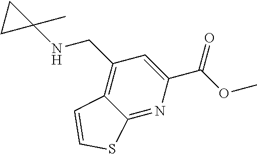 [SMILES: COC(=O)c1cc(CNC2(C)CC2)c2ccsc2n1]

To a solution of 1-methylcyclopropan-1-amine (97.26 mg, 904.03 mol) in MeOH (2 mL) was added TEA (91.48 mg, 904.03 mol), methyl 4-formylthieno[2,3-b]pyridine-6-carboxylate (100 mg, 452.01 mol), CH3COOH (27.14 mg, 452.01 mol) and NaBH3CN (56.81 mg, 904.03 μmol). The mixture was stirred at 25° C. for 2 hr. The reaction mixture was filtered and concentrated under reduced pressure to give a residue, which was purified by prep-HPLC (column: Waters Xbridge BEH C18 100*30 mm*10 um; mobile phase: [H2O (10 mM NH4HCO3)-ACN]; gradient: 25%-55% B over 8.0 min) to give the product (21.5 mg, 17.21% yield) as a white solid. MS(ESI) m/e [M+1]278; ¹H NMR (400 MHz, DMSO-d₆) δ=8.15-8.15 (m, 1H), 8.18-8.08 (m, 1H), 7.70 (d, J=8.0 Hz, 1H), 4.15 (s, 2H), 3.92 (s, 3H), 1.28 (s, 3H), 0.62-0.47 (m, 2H), 0.40-0.25 (m, 2H) ppm.

### Step 5: lithium 4-(((1-methylcyclopropyl)amino)methyl)thieno[2,3-b]pyridine-6-carboxylate

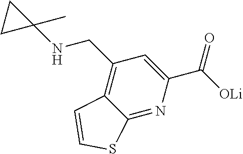

To a solution of methyl 4-(((1-methylcyclopropyl)amino)methyl)thieno[2,3-b]pyridine-6-carboxylate (46.7 mg, 168.99 μmol) in THF (1 mL), MeOH (1 mL), H2O (1 mL) was added LiOH·H2O (8.51 mg, 202.78 mol). The mixture was stirred at 25° C. for 1 hr. The reaction mixture was concentrated under reduced pressure to give a residue, which was used into the next step without further purification to give the product (30 mg, 66.18% yield) as a white solid. MS(ESI) m/e [M+1]263. ¹H NMR (400 MHz, DMSO-d₆) δ=7.92 (s, 1H), 7.79 (d, J=8.0 Hz, 1H), 7.54 (d, J=8.0 Hz, 1H), 4.05 (d, J=5.2 Hz, 2H), 1.28 (s, 4H), 0.58-0.48 (m, 2H), 0.35-0.25 (m, 2H).

### Step 6: N-(5-((1s, 3s)-3-methyl-1-(4-methyl-4H-1,2,4-triazol-3-yl)cyclobutyl)pyridin-3-yl)-4-(((1-methylcyclopropyl)amino)methyl)thieno[2,3-b]pyridine-6-carboxamide

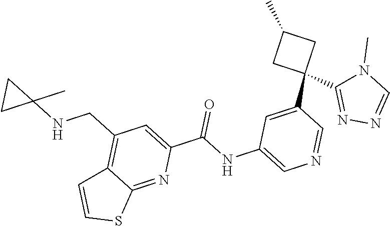 [SMILES: Cn1cnnc1[C@]1(c2cncc(NC(=O)c3cc(CNC4(C)CC4)c4ccsc4n3)c2)C[C@H](C)C1]

To a stirred solution of lithium 4-(((1-methylcyclopropyl)amino)methyl)thieno[2,3-b]pyridine-6-carboxylate (30 mg, 89.47 mol) and 5-((1s, 3s)-3-methyl-1-(4-methyl-4H-1,2,4-triazol-3-yl)cyclobutyl)pyridin-3-amine (21.77 mg, 89.47 mol) in pyridine (1 mL) was added T₃P (170.80 mg, 268.40 mol) at 20° C. The resulting mixture was stirred for 2 h at 20° C. The reaction mixture was quenched by addition H₂O 10 mL at 20° C., and then extracted with EtOAc 30 mL (10 mL*3). The combined organic layers were washed with brine 30 mL (10 mL*3), dried over Na2SO4, filtered and concentrated under reduced pressure to give a residue, which was purified by prep-HPLC (column: Waters Xbridge BEH C18 100*30 mm*10 um; mobile phase: [H2O (10 mM NH4HCO3)-ACN]; gradient: 30%-60% B over 8.0 min) to give the product (9.56 mg, 21.91% yield). MS(ESI) m/e [M+1]488; 1H NMR (400 MHz, DMSO-d6) δ=10.98 (s, 1H), 9.12 (s, 1H), 8.34 (s, 1H), 8.30 (d, J=2.0 Hz, 1H), 8.28 (s, 1H), 8.22 (s, 1H), 8.11 (d, J=6.4 Hz, 1H), 7.73 (d, J=6.4 Hz, 1H), 4.18 (s, 2H), 3.23 (s, 3H), 2.91-2.83 (m, 2H), 2.62-2.55 (m, 3H), 1.30 (s, 3H), 1.10 (d, J=5.6 Hz, 3H), 0.58-0.52 (m, 2H), 0.37-0.32 (m, 2H) ppm.

### Compound B52: 3-chloro-N-(5-((1s, 3s)-3-methyl-1-(4-methyl-4H-1,2,4-triazol-3-yl)cyclobutyl)pyridin-3-yl)-7-(((1-methylcyclobutyl)amino)methyl)-1H-pyrrolo[3,2-b]pyridine-5-carboxamide

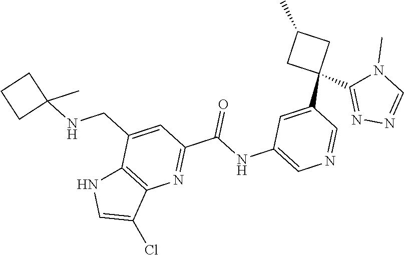 [SMILES: Cn1cnnc1[C@]1(c2cncc(NC(=O)c3cc(CNC4(C)CCC4)c4[nH]cc(Cl)c4n3)c2)C[C@H](C)C1]

### Step A: 3,7-dichloro-1H-pyrrolo[3,2-b]pyridine-5-carbonitrile

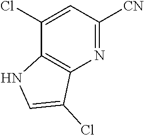 [SMILES: N#Cc1cc(Cl)c2[nH]cc(Cl)c2n1]

To a solution of 7-chloro-1H-pyrrolo[3,2-b]pyridine-5-carbonitrile (2.0 g, 11.236 mmol) in CH3CN (35 mL) was added NCS (1.65 g, 12.360 mmol). The reaction mixture was stirred at 50° C. for 12 h. After completed, the reaction solution was concentrated under vacuum. The residue was washed with H2O (2×30 mL) and free-dried to give the titled compound (1.8 g, 76%). MS: M/e 211 (M+1)⁺

### Step B: 3-chloro-7-vinyl-1H-pyrrolo[3,2-b]pyridine-5-carbonitrile

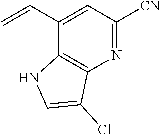

To a solution of 3,7-dichloro-1H-pyrrolo[3,2-b]pyridine-5-carbonitrile (600 mg, 2.844 mmol), potassium trifluoro(vinyl)borate (495 mg, 3.697 mmol), XPhos G2 Pd (224 mg, 0.284 mmol) and Cs₂CO₃ (1.854 g, 5.688 mmol) in dioxane (40 mL) and H₂O (4 mL) was stirred at 80° C. under N₂ for 12 hours. The reaction mixture was quenched with saturated NaCl aq. (20 mL), extracted with EA (45 mL×2), combined, washed brine (30 mL×2), dried and concentrated to dryness. The resulting residue was purified by flash column chromatography (DCM:MeOH=20:1) to give the titled compound (360 mg, 62%). MS: M/e 204 (M+1)⁺

### Step C: 3-chloro-7-formyl-1H-pyrrolo[3,2-b]pyridine-5-carbonitrile

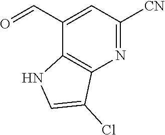 [SMILES: N#Cc1cc(C=O)c2[nH]cc(Cl)c2n1]

To a solution of 3-chloro-7-vinyl-1H-pyrrolo[3,2-b]pyridine-5-carbonitrile (270 mg, 1.330 mmol), K₂OsO₄ 2H₂O (49 mg, 0.133 mmol) in dioxane (30 mL) and H₂O (5 mL) was added NaIO₄ (854 mg, 3.99 mmol). The mixture was stirred at room temperature for 12 hours. After filtration, the solution was concentrated to dryness. The resulting residue was purified by flash column chromatography (DCM:MeOH=15:1) to give the titled compound (170 mg, 62%). MS: M/e 206 (M+1)⁺

### Step D: 3-chloro-7-(((1-methylcyclobutyl)amino)methyl)-1H-pyrrolo[3,2-b]pyridine-5-carbonitrile

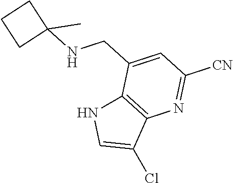 [SMILES: CC1(NCc2cc(C#N)nc3c(Cl)c[nH]c23)CCC1]

To a solution of 3-chloro-7-formyl-1H-pyrrolo[3,2-b]pyridine-5-carbonitrile (80 mg, 0.390 mmol), 1-methylcyclobutan-1-amine hydrochloride (95 mg, 0.780 mmol) and Et₃N (79 mg, 0.780 mmol) in DCM (2 ml) was stirred at room temperature for 1 hour, then added NaBH(OAc)₃ (331 mg, 1.561 mmol). The mixture solution was stirred at 25° C. for 36 hours. The reaction mixture was concentrated under reduced pressure to give the crude product. The crude product was purified by flash column chromatography (DCM:MeOH=20:1) to give the titled compound (86 mg, 81%). MS: M/e 275 (M+1)⁺

### Step E: 3-chloro-7-(((1-methylcyclobutyl)amino)methyl)-1H-pyrrolo[3,2-b]pyridine-5-carboxylic acid

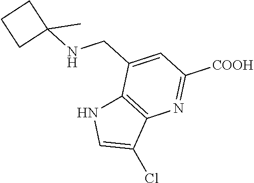 [SMILES: CC1(NCc2cc(C(=O)O)nc3c(Cl)c[nH]c23)CCC1]

To a solution of 3-chloro-7-(((1-methylcyclobutyl)amino)methyl)-1H-pyrrolo[3,2-b]pyridine-5-carbonitrile (86 mg, 0.314 mmol) in H₂SO₄ (1 mL, 6N) was stirred at 100° C. for 16 hours. After completed, added NH₃/MeOH (6N) to adjust PH-8. After filtration, the solution was concentrated under vacuum and free-dried to give the crude product (85 mg, crude). MS: M/e 294 (M+1)⁺

### Step F: 3-chloro-N-(5-((1s, 3s)-3-methyl-1-(4-methyl-4H-1,2,4-triazol-3-yl)cyclobutyl)pyridin-3-yl)-7-(((1-methylcyclobutyl)amino)methyl)-1H-pyrrolo[3,2-b]pyridine-5-carboxamide

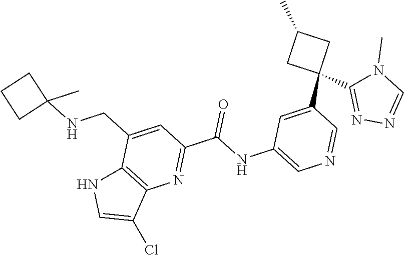 [SMILES: Cn1cnnc1[C@]1(c2cncc(NC(=O)c3cc(CNC4(C)CCC4)c4[nH]cc(Cl)c4n3)c2)C[C@H](C)C1]

To a solution of 3-chloro-7-(((1-methylcyclobutyl)amino)methyl)-1H-pyrrolo[3,2-b]pyridine-5-carboxylic acid (46 mg, 0.157 mmol), 5-((1s, 3s)-3-methyl-1-(4-methyl-4H-1,2,4-triazol-3-yl)cyclobutyl)pyridin-3-amine (38 mg, 0.157 mmol) in pyridine (1 mL) was added T₃P (300 mg, 0.471 mmol, 50% in EA). The mixture solution was stirred for 2 h at 25° C. After completed, the reaction was quenched with aq NH₄Cl (2 mL) and extracted with EA (2×10 mL), dried over Na₂SO₄ and concentrated under vacuum. The crude product was purified by Prep-HPLC to give the title compound (38 mg, 47%). MS: M/e 519 (M+1). ¹H NMR (400 MHz, DMSO) δ 10.61 (s, 1H), 9.07 (d, J=1.6 Hz, 1H), 8.33 (s, 1H), 8.28-8.23 (m, 2H), 8.13 (s, 1H), 7.99 (s, 1H), 4.01 (s, 2H), 3.22 (s, 3H), 2.87 (d, J=3.8 Hz, 2H), 2.63-2.52 (m, 3H), 2.02 (t, J=9.9 Hz, 2H), 1.77-1.62 (m, 4H), 1.25 (s, 3H), 1.08 (d, J=5.3 Hz, 3H) ppm.

### Compound B53:3-chloro-7-(((cyclobutylmethyl)amino)methyl)-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

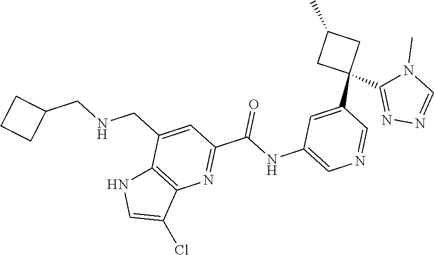 [SMILES: Cn1cnnc1[C@]1(c2cncc(NC(=O)c3cc(CNCC4CCC4)c4[nH]cc(Cl)c4n3)c2)C[C@H](C)C1]

To a solution of 3-chloro-7-(((cyclobutylmethyl)amino)methyl)-1H-pyrrolo[3,2-b]pyridine-5-carboxylic acid (20 mg, 0.068 mmol), 5-((1s, 3s)-3-methyl-1-(4-methyl-4H-1,2,4-triazol-3-yl)cyclobutyl)pyridin-3-amine (17 mg, 0.068 mmol) in pyridine (1 mL) was added T₃P (65 mg, 0.204 mmol, 50% in EA). The mixture solution was stirred for 2 h at 25° C. After completed, the reaction was quenched with aq NH₄Cl (2 mL) and extracted with EA (2×10 mL), dried over Na₂SO₄ and concentrated under vacuum. The crude product was purified by Prep-HPLC to give the title compound (8 mg, 23%). MS: M/e 519 (M+1)⁺. H NMR (400 MHz, DMSO) δ 10.61 (s, 1H), 9.08 (d, J=2.1 Hz, 1H), 8.34 (s, 1H), 8.28 (d, J=1.9 Hz, 1H), 8.26 (d, J=2.1 Hz, 1H), 8.07 (s, 1H), 8.00 (s, 1H), 4.07 (s, 2H), 3.23 (s, 3H), 2.94-2.83 (m, 2H), 2.68-2.55 (m, 5H), 2.46-2.41 (m, 1H), 2.07-1.89 (m, 3H), 1.87-1.72 (m, 2H), 1.69-1.58 (m, 2H), 1.10 (d, J=5.6 Hz, 3H) ppm.

### Compound B54: N-(3-((1s, 3s)-3-(cyanomethyl)-1-(4-methyl-4H-1,2,4-triazol-3-yl)cyclobutyl)phenyl)-3-fluoro-7-(((1-methylcyclopropyl)amino)methyl)-1H-pyrrolo[3,2-b]pyridine-5-carboxamide

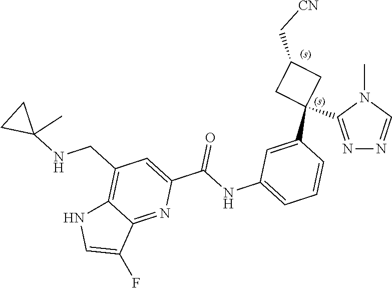 [SMILES: Cn1cnnc1[C@]1(c2cccc(NC(=O)c3cc(CNC4(C)CC4)c4[nH]cc(F)c4n3)c2)C[C@H](CC#N)C1]

To a solution of 3-fluoro-7-(((1-methylcyclopropyl)amino)methyl)-1H-pyrrolo[3,2-b]pyridine-5-carboxylic acid (40 mg, 0.152 mmol), 2-((1s, 3s)-3-(3-aminophenyl)-3-(4-methyl-4H-1,2,4-triazol-3-yl)cyclobutyl)acetonitrile (41 mg, 0.152 mmol) in pyridine (1 mL) was added T₃P (290 mg, 0.456 mmol, 50% in EA). The mixture solution was stirred for 2 h at 25° C. After completed, the reaction was quenched with aq NH₄Cl (2 mL) and extracted with EA (2×10 mL), dried over Na₂SO₄ and concentrated under vacuum. The crude product was purified by Prep-HPLC to give the title compound (30 mg, 38%). MS: M/e 513 (M+1)⁺. H NMR (400 MHz, DMSO) δ 11.37 (s, 1H), 10.52 (s, 1H), 8.32 (s, 1H), 8.05 (s, 1H), 7.95 (d, J=7.5 Hz, 2H), 7.81 (d, J=2.1 Hz, 1H), 7.36 (t, J=8.0 Hz, 1H), 6.98 (d, J=7.8 Hz, 1H), 4.12 (s, 2H), 3.21 (s, 3H), 2.86 (t, J=10.0 Hz, 2H), 2.82-2.63 (m, 5H), 1.29 (s, 3H), 0.59 (d, J=4.9 Hz, 2H), 0.34 (q, J=4.0 Hz, 2H) ppm.

### Compound B55:3-chloro-7-((isobutylamino)methyl)-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

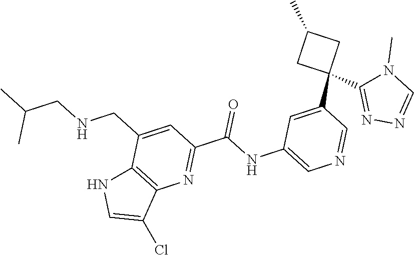 [SMILES: CC(C)CNCc1cc(C(=O)Nc2cncc([C@]3(c4nncn4C)C[C@@H](C)C3)c2)nc2c(Cl)c[nH]c12]

### Step A: 3-chloro-7-((isobutylamino)methyl)-1H-pyrrolo[3,2-b]pyridine-5-carbonitrile

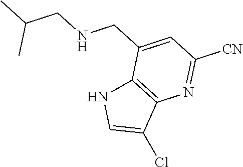 [SMILES: CC(C)CNCc1cc(C#N)nc2c(Cl)c[nH]c12]

To a solution of 3-chloro-7-formyl-1H-pyrrolo[3,2-b]pyridine-5-carbonitrile (100 mg, 0.488 mmol) and 2-methylpropan-1-amine (71 mg, 0.976 mmol) in DCM (1 ml) was stirred at room temperature for 1 hour, then added NaBH(OAc)₃ (414 mg, 1.952 mmol). The mixture solution was stirred at 25° C. for 24 hours. The reaction mixture was concentrated under reduced pressure to give the crude product. The crude product was purified by flash column chromatography (DCM:MeOH=20:1) to give the titled compound (100 mg, 78%). MS: M/e 263 (M+1)⁺

### Step B: 3-chloro-7-((isobutylamino)methyl)-1H-pyrrolo[3,2-b]pyridine-5-carboxylic acid

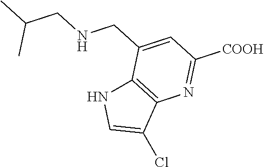 [SMILES: CC(C)CNCc1cc(C(=O)O)nc2c(Cl)c[nH]c12]

To a solution of 3-chloro-7-((isobutylamino)methyl)-1H-pyrrolo[3,2-b]pyridine-5-carbonitrile (100 mg, 0.380 mmol) in H₂SO₄ (1 mL, 6N) was stirred at 100° C. for 16 hours. After completed, added NH₃/MeOH (6N) to adjust PH-8. After filtration, the solution was concentrated under vacuum and free-dried to give the crude product (100 mg, crude). MS: M/e 282 (M+1)⁺

### Step C: 3-chloro-7-((isobutylamino)methyl)-N-(5-((1s, 3s)-3-methyl-1-(4-methyl-4H-1,2,4-triazol-3-yl)cyclobutyl)pyridin-3-yl)-1H-pyrrolo[3,2-b]pyridine-5-carboxamide

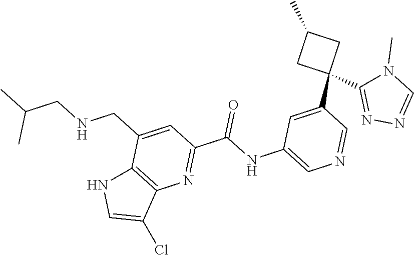 [SMILES: CC(C)CNCc1cc(C(=O)Nc2cncc([C@]3(c4nncn4C)C[C@@H](C)C3)c2)nc2c(Cl)c[nH]c12]

To a solution of 3-chloro-7-((isobutylamino)methyl)-1H-pyrrolo[3,2-b]pyridine-5-carboxylic acid (47 mg, 0.165 mmol), 5-((1s, 3s)-3-methyl-1-(4-methyl-4H-1,2,4-triazol-3-yl)cyclobutyl)pyridin-3-amine (40 mg, 0.165 mmol) in pyridine (1 mL) was added T₃P (315 mg, 0.495 mmol, 50% in EA). The mixture solution was stirred for 2 h at 25° C. After completed, the reaction was quenched with aq NH₄Cl (2 mL) and extracted with EA (2×10 mL), dried over Na₂SO₄ and concentrated under vacuum. The crude product was purified by Prep-HPLC to give the title compound (25 mg, 30%). MS: M/e 507 (M+1). ¹H NMR (400 MHz, DMSO) δ 10.59 (s, 1H), 9.05 (d, J=1.1 Hz, 1H), 8.31 (s, 1H), 8.26 (s, 1H), 8.23 (s, 1H), 8.07 (s, 1H), 7.98 (s, 1H), 4.07 (s, 2H), 3.20 (s, 3H), 2.86 (d, J=3.3 Hz, 2H), 2.63-2.51 (m, 3H), 2.32 (d, J=6.7 Hz, 2H), 1.70 (dt, J=13.2, 6.6 Hz, 1H), 1.07 (d, J=5.0 Hz, 3H), 0.85 (d, J=6.6 Hz, 6H) ppm.

### Compound B56: N-(3-((1s, 3s)-3-(cyanomethyl)-1-(4-methyl-4H-1,2,4-triazol-3-yl)cyclobutyl)phenyl)-1-methyl-7-(((1-methylcyclobutyl)amino)methyl)-1H-pyrrolo[3,2-b]pyridine-5-carboxamide

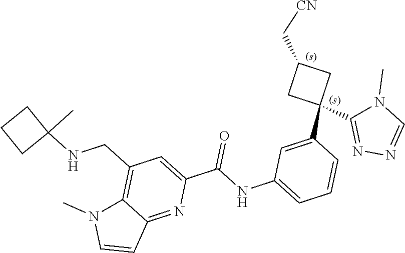 [SMILES: Cn1ccc2nc(C(=O)Nc3cccc([C@]4(c5nncn5C)C[C@@H](CC#N)C4)c3)cc(CNC3(C)CCC3)c21]

### Step A: 1-methyl-7-(((1-methylcyclobutyl)amino)methyl)-1H-pyrrolo[3,2-b]pyridine-5-carbonitrile

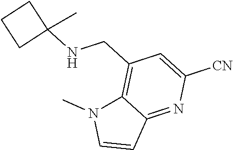 [SMILES: Cn1ccc2nc(C#N)cc(CNC3(C)CCC3)c21]

To a solution of 7-formyl-1-methyl-1H-pyrrolo[3,2-b]pyridine-5-carbonitrile (100 mg, 0.540 mmol), 1-methylcyclobutan-1-amine hydrochloride (131 mg, 1.080 mmol) and Et₃N (108 mg, 1.080 mmol) in DCM (3 ml) was stirred at room temperature for 2 hour, then added NaBH(OAc)₃ (343 mg, 1.620 mmol). The mixture solution was stirred at 25° C. for 24 hours. The reaction mixture was adjusted PH-8 with saturated NaHCO₃ aq, extracted with DCM (10 mL×2), combined, washed brine (5 mL×2), dried and concentrated to dryness. The resulting residue was purified by flash column chromatography (DCM:MeOH=15:1) to give the titled compound (85 mg, 62%). MS: M/e 255 (M+1)⁺

### Step B: 1-methyl-7-(((1-methylcyclobutyl)amino)methyl)-1H-pyrrolo[3,2-b]pyridine-5-carboxylic acid

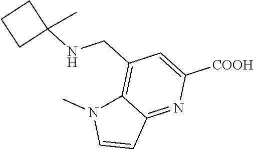 [SMILES: Cn1ccc2nc(C(=O)O)cc(CNC3(C)CCC3)c21]

To a solution of 1-methyl-7-(((1-methylcyclobutyl)amino)methyl)-1H-pyrrolo[3,2-b]pyridine-5-carbonitrile (85 mg, 0.334 mmol) in H₂SO₄ (1 mL, 6N) was stirred at 100° C. for 16 hours. After completed, added NH₃/MeOH (7N) to adjust PH-8. After filtration, the solution was concentrated under vacuum and free-dried to give the crude product (67 mg, crude). MS: M/e 274 (M+1)⁺

### Step C: N-(3-((1s, 3s)-3-(cyanomethyl)-1-(4-methyl-4H-1,2,4-triazol-3-yl)cyclobutyl)phenyl)-1-methyl-7-(((1-methylcyclobutyl)amino)methyl)-1H-pyrrolo[3,2-b]pyridine-5-carboxamide

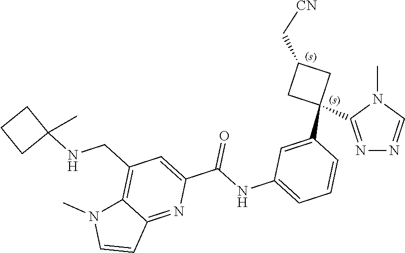 [SMILES: Cn1ccc2nc(C(=O)Nc3cccc([C@]4(c5nncn5C)C[C@@H](CC#N)C4)c3)cc(CNC3(C)CCC3)c21]

To a solution of 1-methyl-7-(((1-methylcyclobutyl)amino)methyl)-1H-pyrrolo[3,2-b]pyridine-5-carboxylic acid (67 mg, 0.245 mmol), 2-((1s, 3s)-3-(3-aminophenyl)-3-(4-methyl-4H-1,2,4-triazol-3-yl)cyclobutyl)acetonitrile (66 mg, 0.245 mmol) in pyridine (3 mL) was added T₃P (468 mg, 0.736 mmol, 50% in EA). The mixture solution was stirred for 2 h at 25° C. After completed, the reaction was quenched with aq NH₄Cl (2 mL) and extracted with EA (2×10 mL), dried over Na₂SO₄ and concentrated under vacuum. The crude product was purified by Prep-HPLC to give the title compound (60 mg, 47%). MS: M/e 523 (M+1)⁺. ¹H NMR (400 MHz, DMSO) δ 10.62 (s, 1H), 8.33 (s, 1H), 7.98 (t, J=7.6 Hz, 3H), 7.73 (s, 1H), 7.35 (t, J=7.8 Hz, 1H), 6.97 (d, J=7.6 Hz, 1H), 6.72 (s, 1H), 4.18 (s, 3H), 4.08 (s, 2H), 3.22 (s, 3H), 2.87 (d, J=8.1 Hz, 2H), 2.80-2.75 (m, 2H), 2.73-2.65 (m, 3H), 2.15-2.02 (m, 2H), 1.84-1.64 (m, 4H), 1.32 (s, 3H) ppm.

### Compound B57: N-(3-((1s, 3s)-3-(cyanomethyl)-1-(4-methyl-4H-1,2,4-triazol-3-yl)cyclobutyl)phenyl)-1-methyl-7-(((1-methylcyclopropyl)amino)methyl)-1H-pyrrolo[3,2-b]pyridine-5-carboxamide

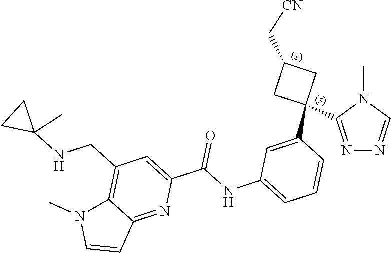 [SMILES: Cn1ccc2nc(C(=O)Nc3cccc([C@]4(c5nncn5C)C[C@@H](CC#N)C4)c3)cc(CNC3(C)CC3)c21]

### Step A: 7-chloro-1-methyl-1H-pyrrolo[3,2-b]pyridine-5-carbonitrile

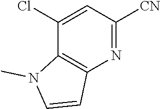

To a solution of 7-chloro-1H-pyrrolo[3,2-b]pyridine-5-carbonitrile (1.25 g, 7.062 mmol) in DMF (45 mL) was added NaH (424 mg, 10.593 mmol) at 0° C. The mixture was stirred at 0° C. for 20 mins, then added CH₃I (1.50 g, 10.593 mmol). The mixture was stirred at room temperature for 6 hours. The reaction mixture was quenched with saturated NH₄Cl aq. (30 mL), extracted with EA (50 mL×2), combined, washed brine (30 mL×2), dried and concentrated to dryness. The resulting residue was purified by flash column chromatography (DCM:MeOH=20:1) to give the titled compound (1.25 g, 93%). MS: M/e 192 (M+1)⁺

### Step B: 1-methyl-7-vinyl-1H-pyrrolo[3,2-b]pyridine-5-carbonitrile

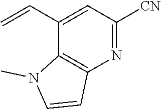

To a solution of 7-chloro-1-methyl-1H-pyrrolo[3,2-b]pyridine-5-carbonitrile (1.25 g, 6.545 mmol), potassium trifluoro(vinyl)borate (1.14 g, 8.508 mmol), XPhos G2 Pd (515 mg, 0.655 mmol) and Cs₂CO₃ (4.27 g, 13.090 mmol) in dioxane (50 mL) and H₂O (5 mL) was stirred at 80° C. under N₂ for 12 hours. The reaction mixture was quenched with saturated NaCl aq. (20 mL), extracted with EA (45 mL×2), combined, washed brine (30 mL×2), dried and concentrated to dryness. The resulting residue was purified by flash column chromatography (DCM:MeOH=20:1) to give the titled compound (1.1 g, 92%). MS: M/e 184 (M+1)⁺

### Step C: 7-formyl-1-methyl-1H-pyrrolo[3,2-b]pyridine-5-carbonitrile

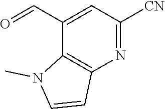

To a solution of 1-methyl-7-vinyl-1H-pyrrolo[3,2-b]pyridine-5-carbonitrile (1.0 g, 5.462 mmol), K₂OsO₄ 2H₂O (201 mg, 0.546 mmol) in dioxane (50 mL) and H₂O (5 mL) was added NaIO₄ (3.506 g, 16.386 mmol). The mixture was stirred at room temperature for 12 hours. After filtration, the solution was concentrated to dryness. The resulting residue was purified by flash column chromatography (DCM:MeOH=15:1) to give the titled compound (800 mg, 79%). MS: M/e 186 (M+1)⁺

### Step D: 1-methyl-7-(((1-methylcyclopropyl)amino)methyl)-1H-pyrrolo[3,2-b]pyridine-5-carbonitrile

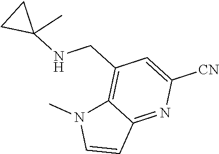 [SMILES: Cn1ccc2nc(C#N)cc(CNC3(C)CC3)c21]

To a solution of 7-formyl-1-methyl-1H-pyrrolo[3,2-b]pyridine-5-carbonitrile (100 mg, 0.540 mmol), 1-methylcyclopropan-1-amine hydrochloride (116 mg, 1.080 mmol) and Et₃N (108 mg, 1.080 mmol) in DCM (3 ml) was stirred at room temperature for 2 hour, then added NaBH(OAc)₃ (343 mg, 1.620 mmol). The mixture solution was stirred at 25° C. for 24 hours. The reaction mixture was adjusted PH-8 with saturated NaHCO₃ aq, extracted with DCM (10 mL×2), combined, washed brine (5 mL×2), dried and concentrated to dryness. The resulting residue was purified by flash column chromatography (DCM:MeOH=15:1) to give the titled compound (80 mg, 62%). MS: M/e 241 (M+1)⁺

### Step E: 1-methyl-7-(((1-methylcyclopropyl)amino)methyl)-1H-pyrrolo[3,2-b]pyridine-5-carboxylic acid

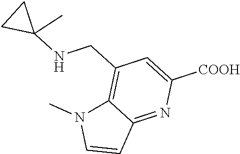 [SMILES: Cn1ccc2nc(C(=O)O)cc(CNC3(C)CC3)c21]

To a solution of 1-methyl-7-(((1-methylcyclopropyl)amino)methyl)-1H-pyrrolo[3,2-b]pyridine-5-carbonitrile (80 mg, 0.333 mmol) in H₂SO₄ (1 mL, 6N) was stirred at 100° C. for 16 hours. After completed, added NH₃/MeOH (7N) to adjust PH-8. After filtration, the solution was concentrated under vacuum and free-dried to give the crude product (74 mg, crude). MS: M/e 260 (M+1)⁺

### Step F: N-(3-((1s, 3s)-3-(cyanomethyl)-1-(4-methyl-4H-1,2,4-triazol-3-yl)cyclobutyl)phenyl)-1-methyl-7-(((1-methylcyclopropyl)amino)methyl)-1H-pyrrolo[3,2-b]pyridine-5-carboxamide

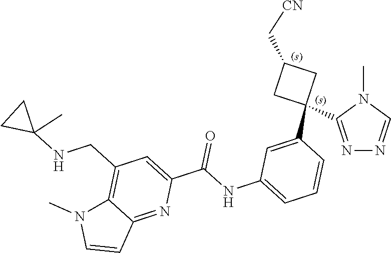 [SMILES: Cn1ccc2nc(C(=O)Nc3cccc([C@]4(c5nncn5C)C[C@@H](CC#N)C4)c3)cc(CNC3(C)CC3)c21]

To a solution of 1-methyl-7-(((1-methylcyclopropyl)amino)methyl)-1H-pyrrolo[3,2-b]pyridine-5-carboxylic acid (74 mg, 0.286 mmol), 2-((1s, 3s)-3-(3-aminophenyl)-3-(4-methyl-4H-1,2,4-triazol-3-yl)cyclobutyl)acetonitrile (76 mg, 0.286 mmol) in pyridine (3 mL) was added T₃P (546 mg, 0.858 mmol, 50% in EA). The mixture solution was stirred for 2 h at 25° C. After completed, the reaction was quenched with aq NH₄Cl (2 mL) and extracted with EA (2×10 mL), dried over Na₂SO₄ and concentrated under vacuum. The crude product was purified by Prep-HPLC to give the title compound (30 mg, 21%). MS: M/e 509 (M+1). ¹H NMR (400 MHz, DMSO) δ 10.60 (s, 1H), 8.32 (s, 1H), 8.02-7.83 (m, 3H), 7.72 (s, 1H), 7.35 (t, J=7.6 Hz, 1H), 6.96 (d, J=7.3 Hz, 1H), 6.71 (s, 1H), 4.20 (s, 2H), 4.15 (s, 3H), 3.21 (s, 3H), 2.86 (d, J=7.8 Hz, 2H), 2.79-2.64 (m, 5H), 1.33 (s, 3H), 0.55 (s, 2H), 0.34 (s, 2H) ppm.

### Compound C1: (S)—N-(3-(3-methyl-1-(4-methyl-4H-1,2,4-triazol-3-yl) cyclobutyl)phenyl)-4-((3-methylpiperidin-1-yl)methyl)-6,7-dihydro-5H-cyclopenta[b]pyridine-2-carboxamide

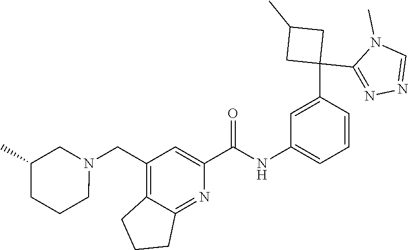 [SMILES: CC1CC(c2cccc(NC(=O)c3cc(CN4CCC[C@H](C)C4)c4c(n3)CCC4)c2)(c2nncn2C)C1]

### Step 1: methyl 4-chloro-6,7-dihydro-5H-cyclopenta[b]pyridine-2-carboxylate

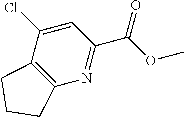

To a solution of 2,4-dichloro-6,7-dihydro-5H-cyclopenta[b]pyridine (3.8 g, 20.2 mmol), NaOAc (2.5 g, 30.3 mmol) in MeOH (80 ml) was added Pd(dppf)Cl₂ (2.9 g, 40 mmol) under N₂. The mixture was stirred at 50° C. for 48 h under CO (50 Psi). The mixture was filtered and concentrated. The crude was purified by column chromatography on sil gel (petroleum ether/EtOAc=50:1 to 5:1) to give product (1.4 g, yield: 32%). ¹H NMR (400 MHz, DMSO-d₆) δ2.13 (quin, J=8 Hz, 2H) 2.99-3.07 (m, 4H) 3.87 (s, 3H) 7.85 (s, 1H) ppm.

### Step 2: methyl 4-vinyl-6,7-dihydro-5H-cyclopenta[b]pyridine-2-carboxylate

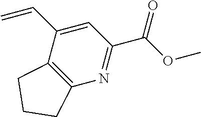

A mixture of methyl 4-chloro-6,7-dihydro-5H-cyclopenta[b]pyridine-2-carboxylate (1 g, 4.7 mmol), potassium trifluoro(vinyl)borate (1.9 g, 14 mmol), K₂CO₃ (1.96 g, 14 mmol) in dioxane (20 mL)/H₂O (6.5 mL) was added Pd(dppf)Cl₂ (345 mg, 472 umol) under N₂. The mixture was stirred at 100° C. under N₂ for 72 hr. The mixture quenched with H₂O (20 mL) and extracted with EtOAc (10 mL*3). The organic layers were concentrated. The residue was purified by column chromatography on sil gel (petroleum ether/EtOAc=10:1 to 1:1) to give the product (600 mg, yield: 62%) as yellow solid. ¹H NMR (400 MHz, DMSO-d₆) δ2.04-2.15 (m, 2H) 2.91-3.04 (m, 4H) 3.86 (s, 3H) 5.64 (d, J=12 Hz, 1H) 6.04 (s, 1H) 6.76-6.87 (m, 1H) 7.92 (s, 1H) ppm.

### Step 3: methyl 4-formyl-6,7-dihydro-5H-cyclopenta[b]pyridine-2-carboxylate

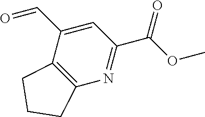

To a solution of methyl 4-vinyl-6,7-dihydro-5H-cyclopenta[b]pyridine-2-carboxylate (500 mg, 2.5 mmol) in dioxane (10 mL)/H₂O (2 mL), was added OsO₄ (62 mg, 246 umol), NaIO₄ (2.1 g, 9.8 mmol). The mixture was stirred at 20° C. for 4 h. The mixture was quenched by adding H₂O (10 ml) and extracted with EA (3 ml*3). The combined organic layers were concentrated to give product (500 mg, crude) used directly. ¹H NMR (400 MHz, DMSO-d₆) δ ppm 2.12-2.22 (m, 2H) 3.02 (t, J=8 Hz, 2H) 3.26-3.32 (t, J=8 Hz, 2H) 3.90 (s, 3H) 8.21 (s, 1H) 10.22 (s, 1H) ppm.

### Step 4: (S)-methyl 4-((3-methylpiperidin-1-yl)methyl)-6,7-dihydro-5H-cyclopenta[b]pyridine-2-carboxylate

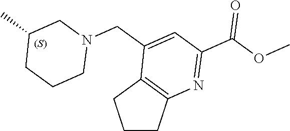

To a solution of methyl 4-formyl-6,7-dihydro-5H-cyclopenta[b]pyridine-2-carboxylate (600 mg, 2.9 mmol), (S)-3-methylpiperidine hydrochloride (436 mg, 3.2 mmol) in DCM (20 mL) was added Et3N (325 mg). After stirring 10 min, NaBH(OAc)₃ (929 mg, 4.3 mmol) was added one portion. The mixture was stirred at 20° C. for 16 h. The reaction was quenched by adding aq. Na2CO3 (10 ml) and extracted with DCM (5 mL*3). The combined organic layers were concentrated. The crude was purified by column chromatography on sil gel (petroleum ether/EtOAc=3:1 to 1:1) to give product (300 mg, yield: 30%). ¹H NMR (400 MHz, DMSO-d₆) δ ppm 0.76-0.91 (m, 4H) 1.45 (br d, J=12 Hz, 1H) 1.52-1.68 (m, 4H) 1.91 (br s, 1H) 2.01-2.14 (m, 2H) 2.61-2.70 (m, 2H) 2.89-2.99 (m, 4H) 3.43 (s, 2H) 3.84 (s, 3H) 7.81 (s, 1H) ppm.

### Step 5: (S)-4-((3-methylpiperidin-1-yl)methyl)-6,7-dihydro-5H-cyclopenta[b]pyridine-2-carboxylic acid

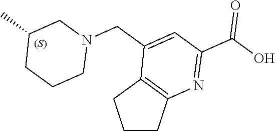

To a solution of (S)-methyl 4-((3-methylpiperidin-1-yl)methyl)-6,7-dihydro-5H-cyclopenta[b]pyridine-2-carboxylate (200 mg, 693 umol) in THF (4 mL)/H₂O (1 mL) was added LiOH·H₂O (145 mg, 3.4 mmol) one portion. The mixture was stirred at 20° C. for 2 h. The mixture was concentrated. The PH was adjust to around 6 by adding aq. HCl (1 M) and concentrated. The crude was purified by prep-HPLC (NH₄HCO₃) to give product (110 mL, yield: 57%). ¹H NMR (400 MHz, DMSO-d₆) δ ppm 0.78-0.85 (m, 4H) 1.38-1.52 (m, 1H) 1.53-1.69 (m, 4H) 1.85-1.97 (m, 1H) 2.02-2.13 (m, 2H) 2.65 (m, 2H) 2.88-2.99 (m, 4H) 3.43 (s, 2H) 7.80 (s, 1H) ppm; MS (ESI) m/e [M+1]+275.

### Step 6: (S)—N-(3-(3-methyl-1-(4-methyl-4H-1,2,4-triazol-3-yl) cyclobutyl)phenyl)-4-((3-methylpiperidin-1-yl)methyl)-6,7-dihydro-5H-cyclopenta[b]pyridine-2-carboxamide

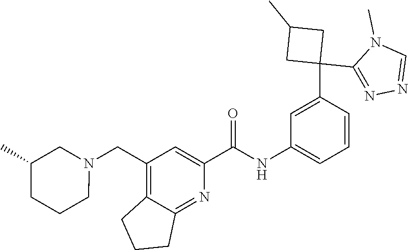 [SMILES: CC1CC(c2cccc(NC(=O)c3cc(CN4CCC[C@H](C)C4)c4c(n3)CCC4)c2)(c2nncn2C)C1]

To a solution of (S)-4-((3-methylpiperidin-1-yl)methyl)-6,7-dihydro-5H-cyclopenta[b]pyridine-2-carboxylic acid (30 mg, 0.11 mmol) and 3-(3-methyl-1-(4-methyl-4H-1,2,4-triazol-3-yl)cyclobutyl)aniline (27 mg, 0.11 mmol) in DMF (5 mL) were added with HATU (50 mg, 0.13 mmol) and DIEA (29 mg, 0.22 mmol). The reaction mixture was stirred at r.t for 2 hrs, then added with water (3 mL), extracted with DCM (5 mL) and washed with brine (5 mL). The organic layer was concentrated and purified by prep. HPLC (Mobile Phase A: 0.03% NH₃·H₂O—H₂O, Mobile Phase B: ACN) to get the product (10 mg, 19%). ¹H NMR (400 MHz, CD₃OD) δ 8.37-8.30 (m, 1H), 8.02 (s, 1H), 7.90 (s, 1H), 7.7. -7.68 (m, 1H), 7.41-7.34 (m, 1H), 7.10-6.95 (m, J=8.0 Hz, 1H), 3.53 (s, 2H), 3.10-3.02 (m, 5H), 2.96-2.91 (m, 2H), 2.82-2.75 (m, 3H), 2.72-2.66 (m, 1H), 2.64-2.45 (m, 3H), 2.74-2.56 (m, 2H), 2.03-1.97 (m, 1H), 1.71-1.56 (m, 5H), 1.16 (d, J=8.0 Hz, 3H), 0.93-0.90 (m, 1H), 0.86 (d, J=4.0 Hz, 3H) ppm. MS: M/e 499 (M+1)+

### Compound C2: 7-methyl-N-(3-((1s,3R)-3-methyl-1-(4-methyl-4H-1,2,4-triazol-3-yl)cyclobutyl)phenyl)-4-(((S)-3-methylpiperidin-1-yl)methyl)-6,7-dihydro-5H-cyclopenta[b]pyridine-2-carboxamide

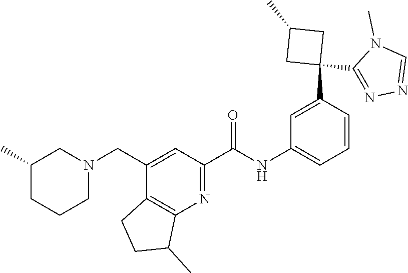 [SMILES: CC1CCc2c(CN3CCC[C@H](C)C3)cc(C(=O)Nc3cccc([C@]4(c5nncn5C)C[C@@H](C)C4)c3)nc21]

### Step 1: ethyl 3-cyano-2-hydroxy-7-methyl-6,7-dihydro-5H-cyclopenta[b]pyridine-4-carboxylate

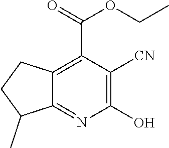 [SMILES: CCOC(=O)c1c(C#N)c(O)nc2c1CCC2C]

To a solution of EtONa (35.7 g, 110 mmol, 21% in EtOH) in EtOH (180 mL) was added diethyl oxalate (13.4 g, 91.7 mmol), 2-methylcyclopentanone (9 g, 91.7 mmol) dropwise. The mixture was stirred at 20° C. for 16 h. Then, 2-cyanoacetamide (7.7 g, 91.7 mmol) was added and the mixture was heated to 80° C. for 7 h. Ethanol was removed under vacuum. The resulting solid was dissolved in H2O (180 mL) and heated to 100° C. for 1 h. The pH was adjust to around 1 by adding AcOH (4 mL) under cooling, where upon a precipitate was formed. The yellow precipitate was collected by filtration and dried under vacuum to give product (12.6 g, yield: 55%) as yellow solid. MS(ESI) m/e [M+1]247; 1H NMR (400 MHz, DMSO-d6) δ=4.42-4.34 (m, 2H), 3.64-3.58 (m, 1H), 3.23-3.12 (m, 1H), 2.89-2.64 (m, 2H), 2.32-2.22 (m, 1H), 1.69-1.57 (m, 1H), 1.33 (t, J=8 Hz, 3H), 1.26-1.19 (m, 3H) ppm.

### Step 2: 2-hydroxy-7-methyl-6,7-dihydro-5H-cyclopenta[b]pyridine-4-carboxylic acid

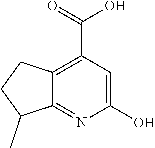

To a solution of ethyl 3-cyano-2-hydroxy-7-methyl-6,7-dihydro-5H-cyclopenta[b]pyridine-4-carboxylate (12.6 g, 51 mmol) in 6M HCl (130 mL). The mixture was stirred for 36 hrs at 115° C. The reaction was poured into H2O (500 mL), and the pH was adjust to around 5 by adding Na2CO3, and extracted with EtOAc (100 mL*3). The combined organic phase was dried over Na2SO4, filtered and concentrated to give the crude product (7.3 g, 74% yield) as yellow solid. MS(ESI) m/e [M+1]194; 1H NMR (400 MHz, DMSO-d6) δ=12.72-12.40 (m, 1H), 6.60 (d, J=2 Hz, 1H), 3.09-2.98 (m, 1H), 2.97-2.87 (m, 1H), 2.83-2.73 (m, 1H), 2.29-2.17 (m, 1H), 1.56 (m, 1H), 1.19 (d, J=8.0 Hz, 3H) ppm.

### Step 3: methyl 2-bromo-7-methyl-6,7-dihydro-5H-cyclopenta[b]pyridine-4-carboxylate

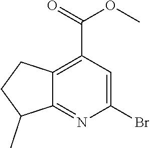

To a solution of 2-hydroxy-7-methyl-6,7-dihydro-5H-cyclopenta[b]pyridine-4-carboxylic acid (5 g, 25.9 mmol) in dry Tol (125 mL) was added POBr3 (74 g, 258 umol) one portion. The mixture was stirred at 100° C. for 16 h under N2. The reaction was quenched by adding MeOH (50 mL) dropwise at 0° C. The reaction was concentrated, and diluted with DCM (100 mL). The pH was adjust to around 5 by adding aq Na2CO3, and extracted with DCM (30 mL*2). The combined organic phase was dried over Na2SO4, filtered and concentrated to give the crude product. The crude was purified by column chromatography (SiO2, Petroleum ether/EtOAc=10:1 to 2:1) to give the product (4 g, 57% yield) as a yellow solid. MS(ESI) m/e [M+1]270; 1H NMR (400 MHz, CDCl3) δ=7.77 (s, 1H), 3.93 (s, 3H), 3.31-3.17 (m, 2H), 3.11-3.00 (m, 1H), 2.46-2.35 (m, 1H), 1.74-1.71 (m, 1H), 1.35 (d, J=9 Hz, 3H) ppm.

### Step 4: (2-bromo-7-methyl-6,7-dihydro-5H-cyclopenta[b]pyridin-4-yl)methanol

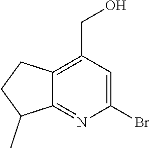

To a solution of methyl 2-bromo-7-methyl-6,7-dihydro-5H-cyclopenta[b]pyridine-4-carboxylate (4 g, 14.8 mmol), CaCl2) (3.3 g, 29.6 mmol) in EtOH (25 mL)/THF (25 mL) was added NaBH4 (2.2 g, 59 mmol) in portions at 0° C. The reaction was quenched by adding H2O (50 mL) extracted with EtOAc (20 mL*3). The combined organic phase was concentrated. The crude was purified by column chromatography (SiO2, Petroleum ether/EtOAc=10:1 to 5:1) to give the product (3.1 g, 86% yield) as a white solid. MS(ESI) m/e [M+1]242; 1H NMR (400 MHz, CDCl3) δ=7.38 (s, 1H), 4.67 (d, J=4 Hz, 2H), 3.25-3.13 (m, 1H), 2.85-2.76 (m, 1H), 2.74-2.64 (m, 1H), 2.45-2.34 (m, 1H), 2.29-2.28 (m, 1H), 2.05 (t, J=8 Hz, 1H), 1.73-1.70 (m, 1H), 1.55-1.55 (m, 1H), 1.33 (d, J=8 Hz, 3H) ppm.

### Step 5: 2-bromo-7-methyl-6,7-dihydro-5H-cyclopenta[b]pyridine-4-carbaldehyde

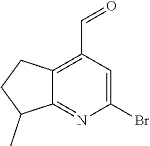

To a solution of (2-bromo-7-methyl-6,7-dihydro-5H-cyclopenta[b]pyridin-4-yl)methanol (3.1 g, 12.8 mmol) in DCM (30 mL) was added DMP (6.5 g, 15.4 mmol) one portion at 0° C. The mixture was stirred for 16 hrs at 15° C. under N2. The reaction was quenched by adding Na2SO3 (20 mL)/NaHCO3 (20 mL) and stirred for 10 min, extracted with DCM (20 mL*3). The combined organic phase was concentrated and purified by column chromatography (SiO2, Petroleum ether/EtOAc=10:1 to 2:1) to give the product (2.7 g, 87% yield) as white solid. MS(ESI) m/e [M+1]240; 1H NMR (400 MHz, CDCl3) δ=10.13 (s, 1H), 7.61 (s, 1H), 3.32-3.19 (m, 2H), 3.12-2.99 (m, 1H), 2.50-2.47 (m, 1H), 1.84-1.71 (m, 1H), 1.37 (d, J=8 Hz, 3H) ppm.

### Step 6: 2-bromo-7-methyl-4-(((S)-3-methylpiperidin-1-yl)methyl)-6,7-dihydro-5H-cyclopenta[b]pyridine

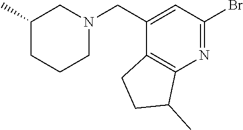 [SMILES: CC1CCc2c(CN3CCC[C@H](C)C3)cc(Br)nc21]

To solution of 2-bromo-7-methyl-6,7-dihydro-5H-cyclopenta[b]pyridine-4-carbaldehyde (2.7 g, 13.8 mmol), (S)-3-methylpiperidine hydrochloride (3.7 g, 27.6 mmol) in DCM (40 mL) was added Et3N (1.4 g, 13.8 mmol). After stirring 10 min, NaBH(OAc)3 (5.8 g, 27.6 mmol) was added. The mixture was stirred for 16 hrs at 15° C. The reaction was quenched by adding aq. NaHCO3 (40 mL) and extracted with DCM (10 mL*3). The combined organic phase was concentrated and purified by column chromatography (SiO2, Petroleum ether/EtOAc=10:1 to 0:1) to give the product (3 g, 82% yield) as a yellow solid. MS(ESI) m/e [M+1]323; 1H NMR (400 MHz, CDCl3) δ=7.30 (s, 1H), 3.34 (s, 2H), 3.24-3.13 (m, 1H), 2.90-2.79 (m, 1H), 2.77-2.65 (m, 3H), 2.40-2.36 (m 1H), 1.97-1.86 (m, 1H), 1.75-1.51 (m, 6H), 1.33 (d, J=8 Hz, 3H), 0.90-0.86 (m, 4H) ppm.

### Step 7: methyl 7-methyl-4-(((S)-3-methylpiperidin-1-yl)methyl)-6,7-dihydro-5H-cyclopenta[b]pyridine-2-carboxylate

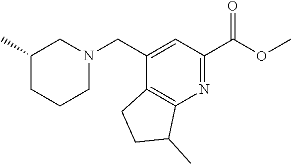

To a solution of 2-bromo-7-methyl-4-(((S)-3-methylpiperidin-1-yl)methyl)-6,7-dihydro-5H-cyclopenta[b]pyridine (1 g, 3.1 mmol) in MeOH (20 mL) was added TEA (626 mg, 6.2 mmol), Pd(dppf)Cl2 (226 mg, 309 umol). The mixture was stirred for 16 hrs at 80° C. under CO (50 Psi). The reaction was concentrated. The crude was purified by column chromatography (SiO2, Petroleum ether/EtOAc=10:1 to 0:1) to give the product (300 mg, 32% yield) as a yellow solid. MS(ESI) m/e [M+1]303;

### Step 8: lithium 7-methyl-4-(((S)-3-methylpiperidin-1-yl)methyl)-6,7-dihydro-5H-cyclopenta[b]pyridine-2-carboxylate

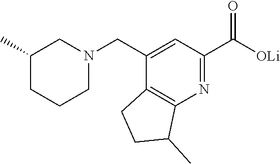

To a solution of methyl 7-methyl-4-(((S)-3-methylpiperidin-1-yl)methyl)-6,7-dihydro-5H-cyclopenta[b]pyridine-2-carboxylate (100 mg, 330 umol) in H2O (1 mL)/THF (1 mL)/MeOH (1 mL) was added LiOH H2O (20 mg, 490 umol). The mixture was stirred for 16 hrs at 15° C. The reaction was concentrated to give product (90 mg, crude) as yellow solid. MS(ESI) m/e [M+1] 289; 1H NMR (400 MHz, DMSO-d6) δ=7.75 (s, 1H), 3.43-3.38 (m, 2H), 3.06-2.97 (m, 1H), 2.90-2.88 (m, 1H), 2.81-2.72 (m, 1H), 2.72-2.61 (m, 2H), 2.66-2.52 (m, 2H), 2.35-2.25 (m, 1H), 1.91-1.89 (m, 1H), 1.69-1.41 (m, 6H), 1.21 (d, J=8 Hz, 3H), 0.82-0.81 (m, 3H) ppm.

### Step 9: 7-methyl-N-(3-((1s,3R)-3-methyl-1-(4-methyl-4H-1,2,4-triazol-3-yl)cyclobutyl)phenyl)-4-(((S)-3-methylpiperidin-1-yl)methyl)-6,7-dihydro-5H-cyclopenta[b]pyridine-2-carboxamide

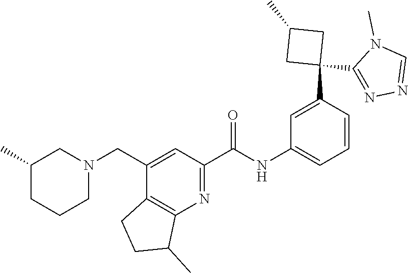 [SMILES: CC1CCc2c(CN3CCC[C@H](C)C3)cc(C(=O)Nc3cccc([C@]4(c5nncn5C)C[C@@H](C)C4)c3)nc21]

To a solution of lithium 7-methyl-4-(((S)-3-methylpiperidin-1-yl)methyl)-6,7-dihydro-5H-cyclopenta[b]pyridine-2-carboxylate (60 mg, 163 umol) in DMF (2 mL) was added DIEA (42 mg, 326 umol), HATU (74 mg, 195 umol) one portion. After stirring 5 min, 3-((1s, 3s)-3-methyl-1-(4-methyl-4H-1,2,4-triazol-3-yl)cyclobutyl)aniline (39 mg, 163 mmol) was added. The mixture was stirred for 16 hrs at 15° C. The reaction was filtered and the filtrates was purified by prep-HPLC (column=Waters Xbridge Prep OBD C18 150*40 mm*10 um; mobile phase=water (NH4HCO3)-ACN, B %=55%-90%; 8 min) to give the product (35 mg, yield: 33%). MS(ESI) m/e [M+1]513; 1H NMR (400 MHz, DMSO-d6) δ=10.36 (s, 1H), 8.27 (s, 1H), 7.90 (d, J=8.0 Hz, 2H), 7.80 (d, J=8.0 Hz, 1H), 7.35 (t, J=8.0 Hz, 1H), 7.01 (d, J=8.0 Hz, 1H), 3.48 (s, 2H), 3.30-3.20 (m, 2H), 3.18 (s, 3H), 3.00-2.96 (m, 1H), 2.90-2.77 (m, 3H), 2.74-2.63 (m, 2H), 2.57-2.52 (m, 3H), 2.42-2.41 (m, 1H), 2.00-1.94 (m, 1H), 1.73-1.55 (m, 5H), 1.50-1.49 (m, 1H), 1.38 (d, J=8.0 Hz, 3H), 1.09 (d, J=4 Hz, 3H), 0.83-0.82 (m, 3H) ppm.

### Compound C3 and compound C4: (S)-7-methyl-N-(3-((1s,3R)-3-methyl-1-(4-methyl-4H-1,2,4-triazol-3-yl)cyclobutyl)phenyl)-4-(((S)-3-methylpiperidin-1-yl)methyl)-6,7-dihydro-5H-cyclopenta[b]pyridine-2-carboxamide or (R)-7-methyl-N-(3-((1s,3S)-3-methyl-1-(4-methyl-4H-1,2,4-triazol-3-yl)cyclobutyl)phenyl)-4-(((S)-3-methylpiperidin-1-yl)methyl)-6,7-dihydro-5H-cyclopenta[b]pyridine-2-carboxamide

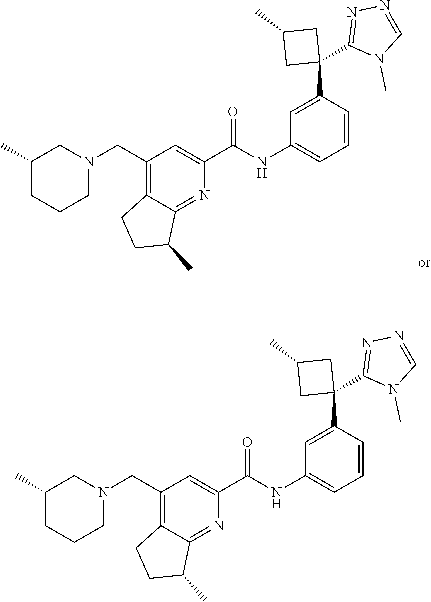 [SMILES: C[C@H]1CCCN(Cc2cc(C(=O)Nc3cccc([C@]4(c5nncn5C)C[C@@H](C)C4)c3)nc3c2CC[C@@H]3C)C1 ; C[C@H]1CCCN(Cc2cc(C(=O)Nc3cccc([C@]4(c5nncn5C)C[C@@H](C)C4)c3)nc3c2CC[C@H]3C)C1]

The 7-methyl-N-(3-((1s,3R)-3-methyl-1-(4-methyl-4H-1,2,4-triazol-3-yl)cyclobutyl)phenyl)-4-(((S)-3-methylpiperidin-1-yl)methyl)-6,7-dihydro-5H-cyclopenta[b]pyridine-2-carboxamide (30 mg, 0.058 mmol) was separated by Prep-SFC (chiral cellulose SA column, 2×25 cm, 5 um; phase A: MTBE; phase B: MeOH/DCM; flow rate: 20 mL/min, 220 nm, 25° C.) to afford the title compound C3 (11 mg) and compound C4 (12 mg).

Compound C3: ¹H NMR (400 MHz, CD₃OD) δ 8.29 (s, 1H), 8.03 (s, 1H), 7.89 (t, J=1.8 Hz, 1H), 7.70 (d, J=8.0 Hz, 1H), 7.40 (t, J=8.0 Hz, 1H), 7.12 (d, J=8.0 Hz, 1H), 3.56 (s, 2H), 3.32-3.21 (m, 3H), 3.11-3.02 (m, 1H), 2.90-2.98 (m, 3H), 2.89-2.63 (m, 4H), 2.62-2.54 (m, 2H), 2.53-2.42 (m, 1H), 2.03 (t, J=9.8 Hz, 1H), 1.82-1.55 (m, 6H), 1.42 (d, J=7.2 Hz, 3H), 1.15 (d, J=6.4 Hz, 3H), 0.89-0.84 (m, 4H) ppm. MS: M/e 513 (M+H)⁺.

Compound C4: ¹H NMR (400 MHz, CD₃OD) δ 8.29 (s, 1H), 8.03 (s, 1H), 7.90 (s, 1H), 7.70 (d, J=8.0 Hz, 1H), 7.40 (t, J=8.0 Hz, 1H), 7.12 (d, J=8.0 Hz, 1H), 3.56 (s, 2H), 3.30-3.21 (m, 3H), 3.12-3.02 (m, 1H), 2.98-2.90 (m, 3H), 2.90-2.64 (m, 4H), 2.63-2.54 (m, 2H), 2.53-2.42 (m, 1H), 2.03 (t, J=10.4 Hz, 1H), 1.80-1.56 (m, 6H), 1.42 (d, J=7.2 Hz, 3H), 1.16 (d, J=6.4 Hz, 3H), 0.98-0.82 (m, 4H). MS: M/e 513 (M+H)⁺.

Compound C5: 7,7-difluoro-N-(3-((1s,3R)-3-methyl-1-(4-methyl-4H-1,2,4-triazol-3-yl)cyclobutyl)phenyl)-4-(((S)-3-methylpiperidin-1-yl)methyl)-6,7-dihydro-5H-cyclopenta[b]pyridine-2-carboxamide

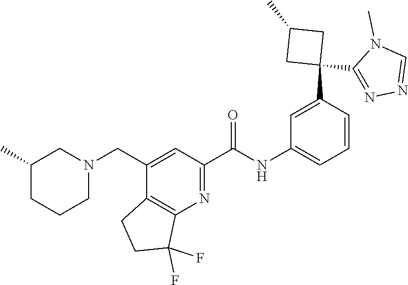 [SMILES: C[C@H]1CCCN(Cc2cc(C(=O)Nc3cccc([C@]4(c5nncn5C)C[C@@H](C)C4)c3)nc3c2CCC3(F)F)C1]

### Step 1: ethyl 3-cyano-2-hydroxy-6,7-dihydro-5H-cyclopenta[b]pyridine-4-carboxylate

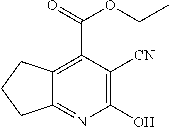 [SMILES: CCOC(=O)c1c(C#N)c(O)nc2c1CCC2]

To a solution of EtONa (92.5 g, 285.3 mmol, 21% in EtOH) in EtOH (400 mL) was added diethyl oxalate (34.8 g, 237.8 mmol), cyclopentanone (20 g, 237.8 mmol) dropwise. The mixture was stirred for 3 hrs at 20° C. Precipitation of slurry was observed. Then 2-cyanoacetamide (20 g, 237.7 mmol) was added and the mixture was heated to 80° C. for 12 h. EtOH was removed and the resulting solid was dissolved in H2O (400 mL) and heated to 100° C. for 1 h. The pH was adjust to around 1 by adding AcOH (4 mL). Precipitation of slurry was observed. The mixture was filtered to give product (38 g, yield: 69%) as yellow solid. MS(ESI) m/e [M+1]233

### Step 2: 2-hydroxy-6,7-dihydro-5H-cyclopenta[b]pyridine-4-carboxylic acid

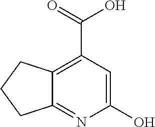 [SMILES: O=C(O)c1cc(O)nc2c1CCC2]

To a solution of ethyl 3-cyano-2-hydroxy-6,7-dihydro-5H-cyclopenta[b]pyridine-4-carboxylate (38 g, 163.6 mmol) in 400 mL HCl (6M). The mixture was stirred for 36 hrs at 115° C. The reaction was poured into H2O (3 L), and filtered to give product (13 g, yield: 44%) as yellow solid. MS(ESI) m/e [M+1]180

### Step 3: methyl 2-bromo-6,7-dihydro-5H-cyclopenta[b]pyridine-4-carboxylate

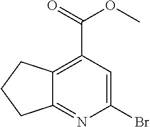

To a solution of 2-hydroxy-6,7-dihydro-5H-cyclopenta[b]pyridine-4-carboxylic acid (12 g, 6732 mmol) in Tol (240 mL). The mixture was stirred for 16 hrs at 100° C. The mixture was cooled to 0° C. was added MeOH (100 mL) dropwise, and concentrated. The mixture was diluted with DCM (300 mL) and washed by Na2CO3. The organic layer was concentrated. The crude was purified by column chromatography (SiO₂, Petroleum ether/EtOAc=100 to 10:1) to give the product (9 g, 52% yield) as a yellow solid. MS(ESI) m/e [M+1]256; ¹H NMR (400 MHz, DMSO-d₆) δ=7.67 (s, 1H), 3.87 (s, 3H), 3.12 (t, J=8.0 Hz, 2H), 2.96 (t, J=8.0 Hz, 2H), 2.13-2.02 (m, 2H) ppm.

### Step 4:2-bromo-4-(methoxycarbonyl)-6,7-dihydro-5H-cyclopenta[b]pyridine 1-oxide

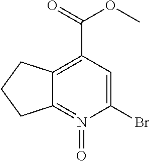

To a solution of methyl 2-bromo-6,7-dihydro-5H-cyclopenta[b]pyridine-4-carboxylate (9 g, 34.2 mmol) in DCM (90 mL) was added mCPBA (9.1 g, 51.3 mmol) in portions. The mixture was stirred for 16 hrs at 20° C. The reaction was filtered and washed by Na₂SO₃, concentrated. The crude was purified by column chromatography (SiO₂, Petroleum ether/EtOAc=10:1 to 2:1) to give the product (7.7 g, 80% yield) as a yellow solid. MS(ESI) m/e [M+1]272; ¹H NMR (400 MHz, DMSO-d₆) δ=8.04 (s, 1H), 3.85 (s, 3H), 3.26 (t, J=8.0 Hz, 2H), 3.02 (t, J=8.0 Hz, 2H), 2.16-2.06 (m, 2H)

### Step 5: methyl 7-acetoxy-2-bromo-6,7-dihydro-5H-cyclopenta[b]pyridine-4-carboxylate

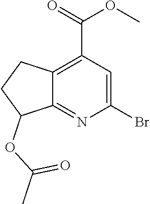 [SMILES: COC(=O)c1cc(Br)nc2c1CCC2OC(C)=O]

To a solution of 2-bromo-4-(methoxycarbonyl)-6,7-dihydro-5H-cyclopenta[b]pyridine 1-oxide (7.7 g, 28.9 mmol) in Ac2O (80 mL). The mixture was stirred for 16 hrs at 100° C. The reaction was concentrated. The pH was adjust to around 5 by adding aq. Na2CO3, and extracted with EA (20 ml*3). The combined organic layer were concentrated. The crude was purified by column chromatography (SiO₂, Petroleum ether/EtOAc=20:1 to 5:1) to give the product (6 g, 67% yield) as a yellow solid. MS(ESI) m/e [M+1]314

### Step 6: 2-bromo-7-hydroxy-6,7-dihydro-5H-cyclopenta[b]pyridine-4-carboxylic acid

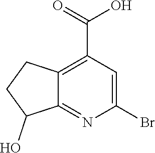 [SMILES: O=C(O)c1cc(Br)nc2c1CCC2O]

To a solution methyl 7-acetoxy-2-bromo-6,7-dihydro-5H-cyclopenta[b]pyridine-4-carboxylate (2.6 g, 8.3 mmol) in MeOH (32 mL) was added NaOH (8 mL, 1 M). The mixture was stirred at 20° C. for 16 h. The reaction was concentrated and the pH was adjust to around 5 by adding 1M HCl. Precipitation of slurry was observed. The mixture was filtered to give product (1.5 g, yield: 65%) as yellow solid. MS(ESI) m/e [M+1]258; ¹H NMR (400 MHz, DMSO-d₆) δ=7.75 (s, 1H), 4.97 (t, J=8.0 Hz, 1H), 3.26-3.14 (m, 1H), 3.01-2.89 (m, 1H), 2.44-2.31 (m, 1H), 1.84-1.83 (m, 1H) ppm.

### Step 7: methyl 2-bromo-7-hydroxy-6,7-dihydro-5H-cyclopenta[b]pyridine-4-carboxylate

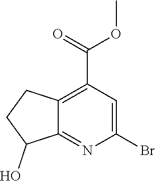

To a solution of 2-bromo-7-hydroxy-6,7-dihydro-5H-cyclopenta[b]pyridine-4-carboxylic acid (2 g, 7.8 mmol) in MeOH (40 mL) was added HCl (0.1 mL). The mixture was stirred at 80° C. for 16 h. The pH was adjust to around 7 by adding aq. NaHCO₃ and concentrated. The crude was diluted with H2O (10 mL) and extracted with EtOAc (4 mL*3) The combined organic phase was concentrated to give product (1.3 g, yield: 61%) as yellow oil. MS(ESI) m/e [M+1]272; ¹H NMR (400 MHz, DMSO-d₆) δ=7.79 (s, 1H), 5.68 (d, J=8 Hz, 1H), 5.01-4.93 (m, 1H), 3.88 (s, 3H), 3.23-3.13 (m, 1H), 2.99-2.88 (m, 1H), 2.43-2.32 (m, 1H), 1.88-1.84 (m, 1H) ppm.

### Step 8: methyl 2-bromo-7-oxo-6,7-dihydro-5H-cyclopenta[b]pyridine-4-carboxylate

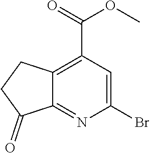

To a solution of methyl 2-bromo-7-hydroxy-6,7-dihydro-5H-cyclopenta[b]pyridine-4-carboxylate (1.3 g, 4.7 mmol) in DCM (20 mL) was added DMP (2.1 g, 9.4 mmol) one portion. The mixture was stirred for 16 hrs at 20° C. The reaction was quenched by adding Na2SO3(10 mL)/NaHCO3 (10 mL). and extracted with DCM (5 mL*3). The combined organic phase was concentrated to give the crude product. The crude was purified by column chromatography (SiO₂, Petroleum ether/EtOAc=10:1 to 2:1) to give the product (1 g, 77% yield) as a yellow solid. MS(ESI) m/e [M+1]270; ¹H NMR (400 MHz, CDCl3) δ=8.17 (s, 1H), 4.03 (s, 3H), 3.48-3.42 (m, 2H), 2.85-2.79 (m, 2H) ppm.

### Step 9: methyl 2-bromo-7,7-difluoro-6,7-dihydro-5H-cyclopenta[b]pyridine-4-carboxylate

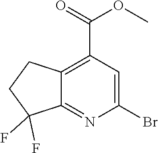 [SMILES: COC(=O)c1cc(Br)nc2c1CCC2(F)F]

To a solution of methyl 2-bromo-7-oxo-6,7-dihydro-5H-cyclopenta[b]pyridine-4-carboxylate (1 g, 3.8 mmol) in BAST (20 mL) was heated to 50° C. for 2 h. The reaction was diluted with DCM (100 mL) and washed by NaHCO₃, concentrated. The was purified by column chromatography (SiO₂, Petroleum ether/EtOAc=10:1 to 2:1) to give the product (400 mg, 64% yield) as a yellow solid MS(ESI) m/e [M+1]292.

¹H NMR (400 MHz, DMSO-d₆) δ=8.11 (s, 1H), 3.90 (s, 3H), 3.29-3.22 (m, 2H), 2.76-2.63 (m, 2H) ppm.

### Step 10: (2-bromo-7,7-difluoro-6,7-dihydro-5H-cyclopenta[b]pyridin-4-yl)methanol

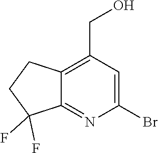 [SMILES: OCc1cc(Br)nc2c1CCC2(F)F]

To a solution of methyl 2-bromo-7,7-difluoro-6,7-dihydro-5H-cyclopenta[b]pyridine-4-carboxylate (480 mg, 1.6 mmol), CaCl₂ (364 mg, 3.3 mmol) in EtOH (10 mL)/THF (10 mL) was added NaBH₄ (248 mg, 6.6 mmol) in portions. The mixture was stirred at 20° C. for 1 h. The mixture was poured into H₂O (100 mL) and extracted with EtOAc (20 mL*3). The combined organic phase was concentrated to give the crude. The crude was purified by column chromatography (SiO₂, Petroleum ether/EtOAc=10:1 to 2:1) to give the product (390 mg, 89% yield) as a white solid. MS(ESI) m/e [M+1]264; ¹H NMR (400 MHz, CDCl3) δ=7.70 (s, 1H), 4.76 (s, 2H), 2.93-2.89 (m, 2H), 2.65-2.52 (m, 2H), 1.94 (s, 1H).

### Step 11: methyl 7,7-difluoro-4-(hydroxymethyl)-6,7-dihydro-5H-cyclopenta[b]pyridine-2-carboxylate

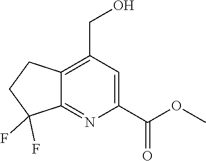 [SMILES: COC(=O)c1cc(CO)c2c(n1)C(F)(F)CC2]

To a solution of (2-bromo-7,7-difluoro-6,7-dihydro-5H-cyclopenta[b]pyridin-4-yl)methanol (290 mg, 1.1 mmol), Et3N (222 mg, 2.2 mmol) in MeOH (20 mL) was added Pd(dppf)Cl2 (80 mg, 109 umol) one portion. The suspension was degassed under vacuum and purged with CO several times. The mixture was stirred under CO (50 psi) at 80° C. for 16 hours. The mixture was filtered and concentrated. The crude was purified by column chromatography (SiO₂, Petroleum ether/EtOAc=10:1 to 2:1) to give the product (100 mg, 39% yield) as a yellow solid. MS(ESI) m/e [M+1]244;

¹H NMR (400 MHz, DMSO-d₆) δ=8.22 (s, 1H), 5.68 (t, J=4 Hz, 1H), 4.66 (d, J=4 Hz, 2H), 3.92 (s, 3H), 3.05-3.02 (m, 2H), 2.76-2.62 (m, 2H) ppm.

### Step 12: methyl 7,7-difluoro-4-formyl-6,7-dihydro-5H-cyclopenta[b]pyridine-2-carboxylate

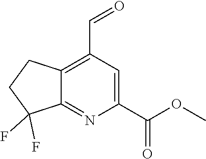 [SMILES: COC(=O)c1cc(C=O)c2c(n1)C(F)(F)CC2]

To a solution of methyl 7,7-difluoro-4-(hydroxymethyl)-6,7-dihydro-5H-cyclopenta[b]pyridine-2-carboxylate (180 mg, 740 umol) in dioxane (10 mL) was added MnO2 (321 mg, 3.7 mmol). The mixture was stirred at 100° C. for 16 hrs. The mixture was filtered and concentrated to give crude product (190 mg, yield: 99%) as brown solid. MS(ESI) m/e [M+1]242; H NMR (400 MHz, DMSO-d₆) δ=10.27 (s, 1H), 8.60 (s, 1H), 3.96 (s, 3H), 3.44-3.38 (m, 2H), 2.83-2.68 (m, 2H) ppm.

### Step 13: (S)-methyl 7,7-difluoro-4-((3-methylpiperidin-1-yl)methyl)-6,7-dihydro-5H-cyclopenta[b]pyridine-2-carboxylate

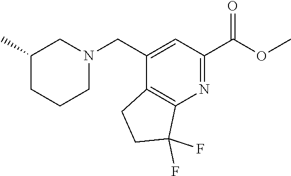

To a solution of methyl 7,7-difluoro-4-formyl-6,7-dihydro-5H-cyclopenta[b]pyridine-2-carboxylate (180 mg, 746 umol), (S)-3-methylpiperidine hydrochloride (148 mg, 1.49 mmol), Et3N (75 mg, 746 umol) in DCM (5 mL). After stirring 5 min, NaBH(OAc)3 (316 mg, 1.5 mmol) was added one portion. The mixture was stirred at 15° C. for 16 hrs. The mixture was concentrated, and purified by prep-TLC (Petroleum ether/EtOAc=1:1) to give the product (50 mg, 19% yield) as a white solid. MS(ESI) m/e [M+1]325; ¹H NMR (400 MHz, DMSO-d₆) δ=8.19-8.10 (m, 1H), 3.92 (s, 3H), 3.58 (s, 2H), 3.18-3.17 (m, 1H), 3.14-3.05 (m, 2H), 2.77-2.63 (m, 5H), 2.02-1.93 (m, 1H), 1.76-1.55 (m, 4H), 0.83 (d, J=6.2 Hz, 4H) ppm.

### Step 14: lithium (S)-7,7-difluoro-4-((3-methylpiperidin-1-yl)methyl)-6,7-dihydro-5H-cyclopenta[b]pyridine-2-carboxylate

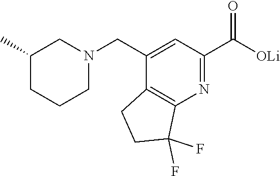

To a solution of (S)-methyl 7,7-difluoro-4-((3-methylpiperidin-1-yl)methyl)-6,7-dihydro-5H-cyclopenta[b]pyridine-2-carboxylate (40 mg, 123.3 umol) and LiOH (123.3 umol) in MeOH (0.5 mL)/THF (0.5 mL)/H2O (0.5 mL). The mixture was stirred at 20° C. for 16 hrs. The reaction was concentrated to give product (39 mg, yield: 99%) as brown solid. MS(ESI) m/e [M+1] 311.

### Step 15: 7,7-difluoro-N-(3-((1s,3R)-3-methyl-1-(4-methyl-4H-1,2,4-triazol-3-yl)cyclobutyl)phenyl)-4-(((S)-3-methylpiperidin-1-yl)methyl)-6,7-dihydro-5H-cyclopenta[b]pyridine-2-carboxamide

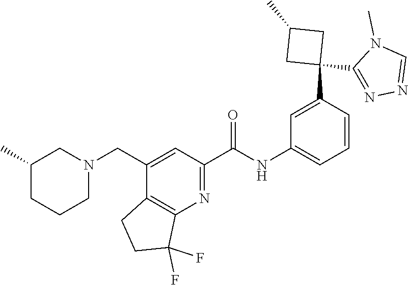 [SMILES: C[C@H]1CCCN(Cc2cc(C(=O)Nc3cccc([C@]4(c5nncn5C)C[C@@H](C)C4)c3)nc3c2CCC3(F)F)C1]

To a solution of lithium (S)-7,7-difluoro-4-((3-methylpiperidin-1-yl)methyl)-6,7-dihydro-5H-cyclopenta[b]pyridine-2-carboxylate (25 mg, 79.1 umol) in DMF (3 mL) was added DIEA (20 mg, 158.1 umol), HATU (36 mg, 94.9 umol), 3-((1s, 3s)-3-methyl-1-(4-methyl-4H-1,2,4-triazol-3-yl)cyclobutyl)aniline (19 mg, 79.1 umol). The mixture was stirred at 15° C. for 16 hrs. The reaction was filtered and purified by prep-HPLC (column=Waters Xbridge Prep OBD C18 150*40 mm*10 um; mobile phase=water (NH4HCO3)-ACN, B %=55%-90%; 8 min) to give product (9.6 mg). MS(ESI) m/e [M+1]535; ¹H NMR (400 MHz, DMSO-d₆) δ=10.45 (s, 1H), 8.29 (s, 1H), 8.23 (s, 1H), 7.91-7.84 (m, 2H), 7.35 (t, J=8 Hz, 1H), 7.02 (d, J=8 Hz, 1H), 3.60 (s, 2H), 3.31 (s, 2H), 3.18 (s, 3H), 3.11 (s, 2H), 2.87-2.79 (m, 2H), 2.78-2.65 (m, 4H), 1.98 (d, J=12 Hz 1H), 1.75-1.44 (m, 6H), 1.08 (d, J=4 Hz, 3H), 0.95-0.79 (m, 4H) ppm.

### Compound C6: N-(3-((1s,3R)-3-(cyanomethyl)-1-(4-methyl-4H-1,2,4-triazol-3-yl)cyclobutyl)phenyl)-7,7-difluoro-4-(((S)-3-methylpiperidin-1-yl)methyl)-6,7-dihydro-5H-cyclopenta[b]pyridine-2-carboxamide

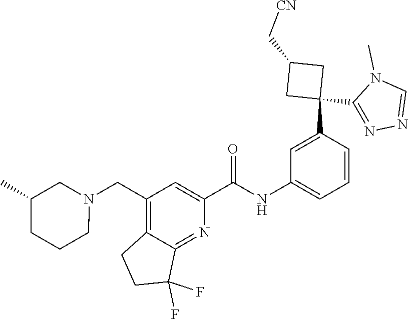 [SMILES: C[C@H]1CCCN(Cc2cc(C(=O)Nc3cccc([C@]4(c5nncn5C)C[C@@H](CC#N)C4)c3)nc3c2CCC3(F)F)C1]

To a solution of lithium (S)-7,7-difluoro-4-((3-methylpiperidin-1-yl)methyl)-6,7-dihydro-5H-cyclopenta[b]pyridine-2-carboxylate (20 mg, 50.6 umol) in DMF (3 mL) was added DIEA (13 mg, 101.2 umol), HATU (23.8 mg, 60.7 umol), 2-((1s, 3s)-3-(3-aminophenyl)-3-(4-methyl-4H-1,2,4-triazol-3-yl)cyclobutyl)acetonitrile (13 mg, 50.6 umol). The mixture was stirred at 15° C. for 16 hrs. The reaction was filtered and purified by prep-HPLC (column=Phenomenex C18 75*30 mm*3 um; mobile phase=water (NH4HCO3)-ACN, B %=40%-70%; 10 min) to give product (1.4 mg, yield: 5%). MS(ESI) m/e [M+1]560; 1H NMR (400 MHz, DMSO-d6) δ=10.48 (s, 1H), 8.32 (s, 1H), 8.23 (s, 1H), 7.94-7.88 (m, 2H), 7.38 (t, J=8.0 Hz, 1H), 7.05 (d, J=8 Hz, 1H), 3.60 (s, 2H), 3.21 (s, 3H), 3.11 (s, 2H), 2.92-2.82 (m, 2H), 2.77-2.66 (m, 8H), 2.04-1.92 (m, 1H), 1.76-1.57 (m, 4H), 1.53-1.50 (m, 1H), 1.24 (br s, 1H), 0.96-0.78 (m, 4H) ppm.

### Compound C7: (S)—N-(3-(3-(cyanomethyl)-1-(4-methyl-4H-1,2,4-triazol-3-yl)cyclobutyl)phenyl)-7,7-dimethyl-4-((3-methylpiperidin-1-yl)methyl)-6,7-dihydro-5H-cyclopenta[b]pyridine-2-carboxamide

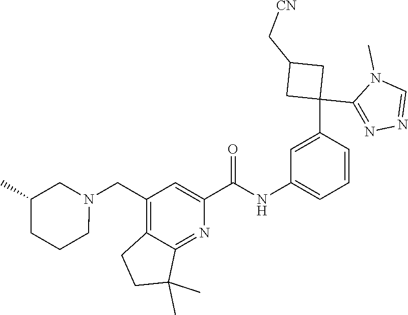 [SMILES: C[C@H]1CCCN(Cc2cc(C(=O)Nc3cccc(C4(c5nncn5C)CC(CC#N)C4)c3)nc3c2CCC3(C)C)C1]

### Step 1: ethyl 2-(3,3-dimethyl-2-oxocyclopentyl)-2-oxoacetate

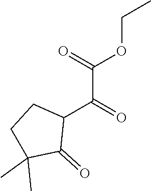

To a solution of EtONa (173.33 g, 534.89 mmol, 21% in ethanol) in ethanol (500 mL) was added 2,2-dimethylcyclopentan-1-one (50 g, 445.74 mmol) and diethyl oxalate (65.14 g, 44.6 mmol) in one portion at 0° C. under N₂. The resulting mixture was stirred at 20° C. for 16 hours. The mixture was used into the next step without further purification.

### Step 2: ethyl (E)-4-amino-3-cyano-2-(3,3-dimethyl-2-oxocyclopentyl)-4-oxobut-2-enoate

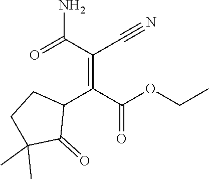 [SMILES: CCOC(=O)/C(=C(\C#N)C(N)=O)C1CCC(C)(C)C1=O]

To a solution of ethyl 2-(3,3-dimethyl-2-oxocyclopentyl)-2-oxoacetate (94.61 g, 445.76 mmol) in ethanol (500 mL) was added 2-cyanoacetamide (37.48 g, 445.76 mmol) and the mixture was heated to 70° C. for 16 h. Ethanol was removed under high vacuum until to give the crude product (124 g, crude), which was used to the next step without purification.

### Step 3: ethyl 3-cyano-2-hydroxy-7,7-dimethyl-6,7-dihydro-5H-cyclopenta[b]pyridine-4-carboxylate

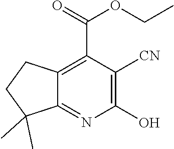 [SMILES: CCOC(=O)c1c(C#N)c(O)nc2c1CCC2(C)C]

The ethyl (E)-4-amino-3-cyano-2-(3,3-dimethyl-2-oxocyclopentyl)-4-oxobut-2-enoate (124.0 g, 445.77 mmol) was dissolved in water (500 mL). The mixture was stirred at 100° C. for 1 hours. Then the aqueous phase was adjusted to pH 5-6 with acetic acid under cooling, where upon a precipitate was formed. The orange-colored precipitate was collected by filtration and dried under vacuum to give product (18 g,). MS(ESI) m/e [M+1]261; 1H NMR (400 MHz, CDCl3) δ=4.47 (q, J=7.2 Hz, 2H), 2.90 (t, J=7.2 Hz, 2H), 2.02 (t, J=7.2 Hz, 2H), 1.49-1.37 (m, 9H) ppm.

### Step 4: 2-hydroxy-7,7-dimethyl-6,7-dihydro-5H-cyclopenta[b]pyridine-4-carboxylic acid

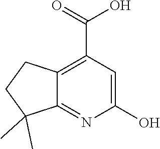

The ethyl 3-cyano-2-hydroxy-7,7-dimethyl-6,7-dihydro-5H-cyclopenta[b]pyridine-4-carboxylate (5 g, 19.21 mmol) was dissolved in aq.HCl (6M) (50 mL). The mixture was stirred at 115° C. for 16 hours. To this was added ice water (300 mL), where upon a precipitate was formed. The orange-colored precipitate was collected by filtration and dried under vacuum to give product (3 g, yield 75.36%). MS(ESI) m/e [M+1]208; 1H NMR (400 MHz, DMSO-d6) δ=6.62 (s, 1H), 2.91-2.82 (m, 2H), 1.86 (t, J=7.2 Hz, 2H), 1.22 (s, 6H) ppm.

### Step 5: methyl 2-bromo-7,7-dimethyl-6,7-dihydro-5H-cyclopenta[b]pyridine-4-carboxylate

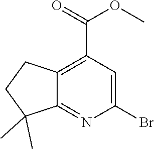

To a solution of 2-hydroxy-7,7-dimethyl-6,7-dihydro-5H-cyclopenta[b]pyridine-4-carboxylic acid (3 g, 14.48 mmol) in Tol. (60 mL) was added POBr₃ (20.75 g, 72.38 mmol) in one portion. The mixture was stirred for 16 hrs at 100° C. under N₂. The reaction mixture was quenched by addition MeOH 20 mL at 0° C., then the solvent was removed. The mixture was adjusted to pH 7-8 with aq. Na₂CO₄, and extracted with DCM 30 mL (10 mL*3). The combined organic layers were washed with brine 30 mL (10 mL*3), dried over Na₂SO₄, filtered and concentrated under reduced pressure to give a residue, which was purified by column chromatography (SiO₂, PE:EA=100/1, 1/1) to give the product (3 g, 72.93% yield) as a off-white solid. MS(ESI) m/e [M+1]284; 1H NMR (400 MHz, DMSO-d6) δ=7.69 (s, 1H), 3.87 (s, 3H), 3.09 (t, J=7.2 Hz, 2H), 2.02-1.89 (m, 2H), 1.22 (s, 6H) ppm.

### Step 6: (2-bromo-7,7-dimethyl-6,7-dihydro-5H-cyclopenta[b]pyridin-4-yl)methanol

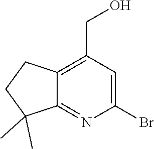

To a mixture of methyl 2-bromo-7,7-dimethyl-6,7-dihydro-5H-cyclopenta[b]pyridine-4-carboxylate (2.5 g, 8.80 mmol) and CaCl₂ (1.95 g, 17.60 mmol) in EtOH (15 ml) and THF (15 ml) was added NaBH4 (1.33 g, 35.19 mmol) in portions at 0° C. The mixture was stirred at 0° C. for 2 h. The mixture was poured into water (30 mL) and extracted with EtOAc (60 mL, 20 mL). The cooled reaction mixture was partitioned between water and EtOAc, and the separated organic layer was dried, filtered, concentrated was purified on column chromatography (SiO₂, PE:EA=100/1, 1/1) to give the product (2 g, 92% yield). MS(ESI) m/e [M+1]256; 1H NMR (400 MHz, DMSO-d6) δ=7.39 (s, 1H), 5.52 (t, J=5.6 Hz, 1H), 4.54 (d, J=5.6 Hz, 2H), 2.77 (t, J=7.2 Hz, 2H), 1.98 (t, J=7.2 Hz, 2H), 1.25 (s, 6H) ppm.

### Step 7: methyl 4-(hydroxymethyl)-7,7-dimethyl-6,7-dihydro-5H-cyclopenta[b]pyridine-2-carboxylate

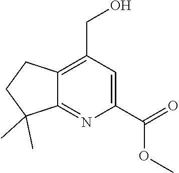

A mixture of (2-bromo-7,7-dimethyl-6,7-dihydro-5H-cyclopenta[b]pyridin-4-yl)methanol (2 g, 7.81 mmol), Pd(dppf)Cl2 (1.14 g, 1.56 mmol), and TEA (1.58 g, 15.62 mmol) in MeOH (20 mL) was degassed and purged with CO for 3 times, The mixture was stirred under CO (50 psi) at 80° C. for 5 hours. The reaction mixture was filtered and the filter was concentrated. The crude product was purified by column chromatography (SiO₂, Petroleum ether/Ethyl acetate=100/1 to 0/1) to give product (1.8 g, 78% yield) as yellow solid. MS(ESI) m/e [M+1]236; 1H NMR (400 MHz, DMSO-d6) δ=7.93 (s, 1H), 5.48 (t, J=5.6 Hz, 1H), 4.54 (d, J=5.6 Hz, 2H), 3.86 (s, 3H), 2.82 (t, J=7.2 Hz, 2H), 1.96 (t, J=7.2 Hz, 2H), 1.22 (s, 6H) ppm.

### Step 8: methyl 4-formyl-7,7-dimethyl-6,7-dihydro-5H-cyclopenta[b]pyridine-2-carboxylate

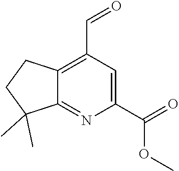

To a solution of methyl 4-(hydroxymethyl)-7,7-dimethyl-6,7-dihydro-5H-cyclopenta[b]pyridine-2-carboxylate (800 mg, 3.40 mmol) in Dioxane (10 ml) was added MnO₂ (1.48 g, 17.00 mmol) in one portion at 20° C. The mixture was stirred at 100° C. for 16 h. The reaction mixture was filtered and the filter was concentrated. The crude product was purified by column chromatography (SiO₂, Petroleum ether/Ethyl acetate=100/1 to 0/1) to give product (400 mg, 50.42% yield) as yellow solid. MS(ESI) m/e [M+1]234; 1H NMR (400 MHz, DMSO-d6) δ=10.23 (s, 1H), 8.23 (s, 1H), 3.91 (s, 3H), 3.25 (t, J=7.2 Hz, 2H), 2.04 (t, J=7.2 Hz, 2H), 1.27 (s, 6H) ppm.

### Step 9: methyl (S)-7,7-dimethyl-4-((3-methylpiperidin-1-yl)methyl)-6,7-dihydro-5H-cyclopenta[b]pyridine-2-carboxylate

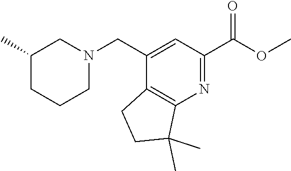

To a mixture of methyl 4-formyl-7,7-dimethyl-6,7-dihydro-5H-cyclopenta[b]pyridine-2-carboxylate (200 mg, 857.40 mol), (S)-3-methylpiperidine hydrochloride (232.59 mg, 1.71 mmol) and TEA (173.52 mg, 1.71 mmol) in DCM (3 mL) was added NaBH(OAc)₃ (363.44 mg, 1.71 mmol) in one portion at 20° C. The mixture was stirred at 20° C. for 12 h. The mixture was poured into water (1 mL) and extracted with DCM (2 mL, 1 mL). The combined organic phase was washed with brine (1 mL), dried over Na₂SO₄, filtered and concentrated to give the residue, which was purified by column chromatography (SiO₂, Petroleum ether/Ethyl acetate=100/1 to 0/1) to give the product (140 mg, 51% yield). MS(ESI) m/e [M+1]317; 1H NMR (400 MHz, DMSO-d6) δ=7.83 (s, 1H), 3.86 (s, 3H), 3.45 (s, 2H), 2.88 (t, J=7.2 Hz, 2H), 2.71-2.60 (m, 2H), 1.95 (brt, J=7.2 Hz, 3H), 1.64 (br d, J=9.6 Hz, 5H), 1.22 (s, 6H), 0.92-0.79 (m, 4H) ppm.

### Step 10: lithium (S)-7,7-dimethyl-4-((3-methylpiperidin-1-yl)methyl)-6,7-dihydro-5H-cyclopenta[b]pyridine-2-carboxylate

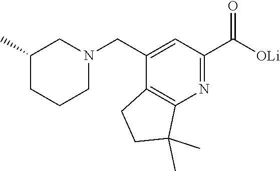

To a solution of methyl (S)-7,7-dimethyl-4-((3-methylpiperidin-1-yl)methyl)-6,7-dihydro-5H-cyclopenta[b]pyridine-2-carboxylate (140 mg, 442.42 μmol) in MeOH/THF/H₂O (0.5 mL/0.5 mL/0.5 mL) was added LiOH·H₂O (27.85 mg, 663.64 mol). The mixture was stirred for 12 hr at rt. The mixture was filtered and concentrated to give the crude product (80 mg, crude), which was used to the next step without purification. MS(ESI) m/e [M+1]303; 1H NMR (400 MHz, DMSO-d6) δ=7.80 (s, 1H), 3.41 (s, 2H), 2.81 (t, J=7.2 Hz, 2H), 2.73-2.62 (m, 2H), 1.97-1.83 (m, 3H), 1.71-1.39 (m, 5H), 1.17 (s, 6H), 0.81 (br d, J=5.6 Hz, 4H) ppm.

### Step 11: (S)—N-(3-(3-(cyanomethyl)-1-(4-methyl-4H-1,2,4-triazol-3-yl)cyclobutyl)phenyl)-7,7-dimethyl-4-((3-methylpiperidin-1-yl)methyl)-6,7-dihydro-5H-cyclopenta[b]pyridine-2-carboxamide

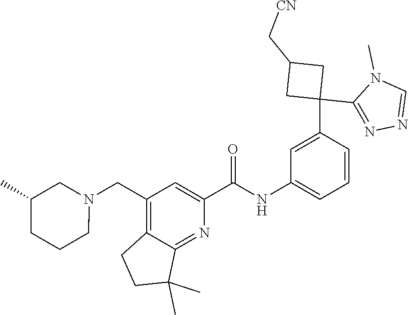 [SMILES: C[C@H]1CCCN(Cc2cc(C(=O)Nc3cccc(C4(c5nncn5C)CC(CC#N)C4)c3)nc3c2CCC3(C)C)C1]

To a solution of lithium (S)-7,7-dimethyl-4-((3-methylpiperidin-1-yl)methyl)-6,7-dihydro-5H-cyclopenta[b]pyridine-2-carboxylate (20 mg, 54.99 μmol) in DMF (1 mL) was added DIEA (14.21 mg, 109.98 μmol) and HATU (25.09 mg, 65.99 μmol). The mixture was stirred for 5 mins at rt, then 2-(3-(3-aminophenyl)-3-(4-methyl-4H-1,2,4-triazol-3-yl)cyclobutyl)acetonitrile (15 mg, 54.99 mol) was added. The mixture was stirred for 12 hrs at rt. The mixture was poured into water (1 mL) and extracted with DCM (2 mL, 1 mL). The combined organic phase was filtered and concentrated to give the residue, which was purified by prep-HPLC (column=Waters Xbridge BEH C18 (100*30 mm*10 um); mobile phase=water (NH4HCO3)-ACN, B %=50%-80%; 8 min) to give the product (9 mg). MS(ESI) m/e [M+1]552; 1H NMR (400 MHz, DMSO-d6) δ=10.31 (d, J=9.2 Hz, 1H), 8.39 (s, 0.5H), 8.31 (s, 0.5H), 7.93-7.85 (m, 1.5H), 7.81-7.66 (m, 1.5H), 7.38 (q, J=7.6 Hz, 1H), 7.09 (br d, J=8.0 Hz, 0.5H), 6.97 (d, J=7.6 Hz, 0.5H), 3.48 (s, 2H), 3.22 (d, J=10.4 Hz, 3H), 3.16-3.07 (m, 1H), 2.95-2.85 (m, 3H), 2.80-2.62 (m, 6H), 2.46 (br s, 1H), 2.04-1.88 (m, 3H), 1.72-1.40 (m, 5H), 1.32 (d, J=2.0 Hz, 6H), 0.82 (br d, J=6.0 Hz, 4H) ppm.

### Compound C8: N-(3-((1r,3S)-3-cyano-1-((4-methyl-4H-1,2,4-triazol-3-yl)methyl)cyclobutyl)phenyl)-7,7-dimethyl-4-(((S)-3-methylpiperidin-1-yl)methyl)-6,7-dihydro-5H-cyclopenta[b]pyridine-2-carboxamide

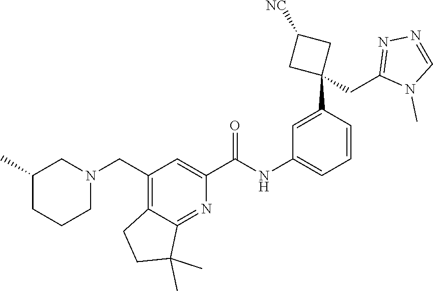 [SMILES: C[C@H]1CCCN(Cc2cc(C(=O)Nc3cccc([C@]4(Cc5nncn5C)C[C@@H](C#N)C4)c3)nc3c2CCC3(C)C)C1]

To a solution of lithium (S)-7,7-dimethyl-4-((3-methylpiperidin-1-yl)methyl)-6,7-dihydro-5H-cyclopenta[b]pyridine-2-carboxylate (80 mg, 168.64 mol) in DMF (1 mL) was added DIEA (43.59 mg, 337.28 mol) and HATU (76.95 mg, 202.37 mol). The mixture was stirred for 5 mins at rt, then (1r, 3r)-3-(3-aminophenyl)-3-((4-methyl-4H-1,2,4-triazol-3-yl)methyl)cyclobutane-1-carbonitrile (45.08 mg, 168.64 mol) was added. The mixture was stirred for 12 hrs at rt. The mixture was poured into water (1 mL) and extracted with DCM (2 mL, 1 mL). The combined organic phase was filtered and concentrated to give the residue, which was purified by prep-HPLC (column=Waters Xbridge BEH C18 (100*30 mm*10 um); mobile phase=water (NH₄HCO3)-ACN, B %=50%-80%; 10 min) to give the product (32.9 mg, 35.36% yield); MS(ESI) m/e [M+1]552; ¹H NMR (400 MHz, DMSO-d6) δ=10.23 (s, 1H), 8.18 (s, 1H), 7.92 (s, 1H), 7.76 (dd, J=1.2, 8.0 Hz, 1H), 7.54 (s, 1H), 7.27 (t, J=8.0 Hz, 1H), 6.69 (d, J=7.6 Hz, 1H), 3.49 (s, 2H), 3.28 (s, 3H), 2.95-2.83 (m, 6H), 2.80 (s, 3H), 2.74-2.63 (m, 2H), 2.04-1.88 (m, 3H), 1.72-1.56 (m, 4H), 1.55-1.43 (m, 1H), 1.33 (s, 6H), 0.91-0.83 (m, 4H) ppm.

### Compound C9: N-(3-((1s,3R)-3-(cyanomethyl)-1-(4-methyl-4H-1,2,4-triazol-3-yl)cyclobutyl)phenyl)-7,7-dimethyl-4-(((S)-3-methylpiperidin-1-yl)methyl)-6,7-dihydro-5H-cyclopenta[b]pyridine-2-carboxamide

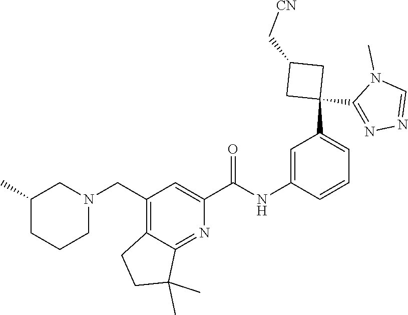 [SMILES: C[C@H]1CCCN(Cc2cc(C(=O)Nc3cccc([C@]4(c5nncn5C)C[C@@H](CC#N)C4)c3)nc3c2CCC3(C)C)C1]

To a solution of lithium (S)-7,7-dimethyl-4-((3-methylpiperidin-1-yl)methyl)-6,7-dihydro-5H-cyclopenta[b]pyridine-2-carboxylate (80 mg, 168.64 μmol) in DMF (1 mL) was added DIEA (43.59 mg, 337.28 mol) and HATU (76.95 mg, 202.37 mol). The mixture was stirred for 5 mins at rt, then 2-((1s, 3s)-3-(3-aminophenyl)-3-(4-methyl-4H-1,2,4-triazol-3-yl)cyclobutyl)acetonitrile (45.08 mg, 168.64 mol) was added. The mixture was stirred for 12 hrs at rt. The mixture was poured into water (1 mL) and extracted with DCM (2 mL, 1 mL). The combined organic phase was filtered and concentrated to give the residue, which was purified by prep-HPLC (column=Waters Xbridge Prep OBD C18 (150*40 mm*0 um); mobile phase=water (NH4HCO3)-ACN, B %=50%-80%; 8 min) to give the product (26.1 mg, 28.05% yield). MS(ESI) m/e [M+1]552; 1H NMR (400 MHz, DMSO-d6) δ=10.32 (s, 1H), 8.31 (s, 1H), 7.91 (s, 1H), 7.87 (s, 1H), 7.80-7.73 (m, 1H), 7.39 (t, J=8.0 Hz, 1H), 7.08 (d, J=8.0 Hz, 1H), 3.48 (s, 2H), 3.21 (s, 3H), 2.96-2.83 (m, 4H), 2.81-2.63 (m, 7H), 2.02-1.88 (m, 3H), 1.71-1.42 (m, 5H), 1.33 (s, 6H), 0.92-0.78 (m, 4H) ppm.

### Compound C1O: 3,3-dimethyl-7-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide

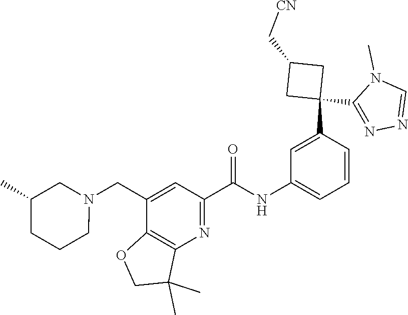 [SMILES: C[C@H]1CCCN(Cc2cc(C(=O)Nc3cccc([C@]4(c5nncn5C)C[C@@H](CC#N)C4)c3)nc3c2OCC3(C)C)C1]

### Step 1: 5-hydroxy-6-iodopyridine-2-carboxylic acid

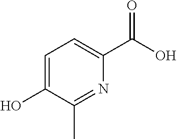

To a stirred solution of 5-hydroxypyridine-2-carboxylic acid (1 g, 6.829 mmol, 1 equiv, 95%) in NH₃·H2O (30 mL, 770.416 mmol, 112.81 equiv) were added a solution of KI (5.97 g, 34.145 mmol, 5.00 equiv, 95%) and I₂ (1.82 g, 6.829 mmol, 1.00 equiv, 95%) in H₂O (40 mL, 4351.849 mmol, 637.26 equiv, 98%) dropwise at room temperature. The resulting mixture was stirred for overnight at room temperature. The mixture was acidified to pH 3 with HCl (aq.). The resulting mixture was extracted with EtOAc (3×150 mL). The combined water layers were concentrated under reduced pressure. The crude product was used in the next step directly without further purification. This resulted in 5-hydroxy-6-iodopyridine-2-carboxylic acid (1 g, 55.20%)

### Step 2: methyl 5-hydroxy-6-iodopyridine-2-carboxylate

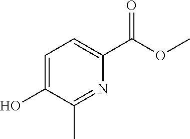

To a stirred solution of 5-hydroxy-6-iodopyridine-2-carboxylic acid (1 g, 3.770 mmol, 1 equiv, 99.9%) in MeOH was added SOCl₂ (1.33 mL, 18.021 mmol, 4.78 equiv, 98%) dropwise at 0° C. The resulting mixture was stirred for 2 h at 70° C. The mixture was allowed to cool down to rt. The resulting mixture was concentrated under reduced pressure. The residue was purified by reverse flash chromatography with the following conditions: column, C18 silica gel; mobile phase, MeCN in Water (0.1% TFA), 40% to 50% gradient in 4 min; detector, UV 220 nm. This resulted in methyl 5-hydroxy-6-iodopyridine-2-carboxylate (600 mg, 53.11%). MS: M/e 280 (M+1).

### Step 3: methyl 6-iodo-5-[(2-methylprop-2-en-1-yl)oxy]pyridine-2-carboxylate

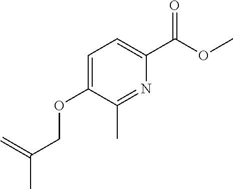 [SMILES: C=C(C)COc1ccc(C(=O)OC)nc1C]

A solution of methyl 5-hydroxy-6-iodopyridine-2-carboxylate (500 mg, 1.668 mmol, 1 equiv, 93.1%), 3-bromo-2-methylprop-1-ene (250 mg, 1.815 mmol, 1.09 equiv, 98%) and K₂CO₃ (609 mg, 4.186 mmol, 2.51 equiv, 95%) in acetone was stirred for overnight at 60° C. under nitrogen atmosphere. The resulting mixture was concentrated under reduced pressure. The residue was purified by silica gel column chromatography, eluted with PE/EA (2:1) to afford methyl 6-iodo-5-[(2-methylprop-2-en-1-yl)oxy]pyridine-2-carboxylate (500 mg, 89.88%).

### Step 4: methyl 3,3-dimethyl-2H-furo[3,2-b]pyridine-5-carboxylate

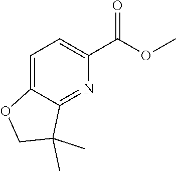

A solution of methyl 6-iodo-5-[(2-methylprop-2-en-1-yl)oxy]pyridine-2-carboxylate (500 mg, 1.499 mmol, 1 equiv, 99.9%), n-Bu₃SnH (689 mg, 2.249 mmol, 1.50 equiv, 95%) and AIBN (26 mg, 0.150 mmol, 0.10 equiv, 95%) in benzene was stirred for overnight at 80° C. under nitrogen atmosphere. The mixture was allowed to cool down to room temperature. The resulting mixture was added 10% KF(aq.) (5 mL) at rt. The resulting mixture was stirred for 3.5 h at room temperature. The resulting mixture was extracted with EtOAc (3×50 mL). The combined organic layers were dried over anhydrous Na2SO4. After filtration, the filtrate was concentrated under reduced pressure. The residue was purified by silica gel column chromatography, eluted with PE/EA (5:1) to afford methyl 3,3-dimethyl-2H-furo[3,2-b]pyridine-5-carboxylate (240 mg, 77.16%).

### Step 5: methyl 3,3-dimethyl-4-oxo-2H-4lambda5-furo[3,2-b]pyridine-5-carboxylate

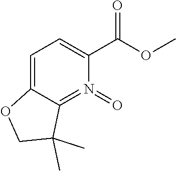 [SMILES: COC(=O)c1ccc2c([n+]1[O-])C(C)(C)CO2]

To a stirred solution of methyl 3,3-dimethyl-2H-furo[3,2-b]pyridine-5-carboxylate (160 mg, 0.771 mmol, 1 equiv, 99.9%) and Urea hydrogen peroxide (220 mg, 2.222 mmol, 2.88 equiv, 95%) in DCM was added TFAA (0.32 mL, 2.236 mmol, 2.9 equiv, 98%) dropwise at 0° C. The resulting mixture was stirred for overnight at room temperature. The mixture was added H2O (30 mL). The resulting mixture was extracted with CH₂Cl₂ (3×30 mL). The combined organic layers were dried over anhydrous Na₂SO₄. After filtration, the filtrate was concentrated under reduced pressure. The residue was purified by silica gel column chromatography, eluted with PE/EA (1:2) to afford methyl 3,3-dimethyl-4-oxo-2H-4lambda5-furo[3,2-b]pyridine-5-carboxylate (140 mg, 81.23%).

### Step 6: methyl 7-chloro-3,3-dimethyl-2H-furo[3,2-b]pyridine-5-carboxylate

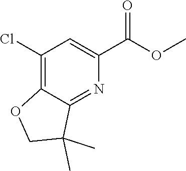 [SMILES: COC(=O)c1cc(Cl)c2c(n1)C(C)(C)CO2]

To a stirred solution of methyl 3,3-dimethyl-4-oxo-2H-4lambda5-furo[3,2-b]pyridine-5-carboxylate (140 mg, 0.627 mmol, 1 equiv, 99.9%) in DMF was added POCl3 (0.12 mL, 1.262 mmol, 2.01 equiv, 98%) dropwise at 0° C. The resulting mixture was stirred for 2 h at 100° C. under nitrogen atmosphere. The mixture was allowed to cool down to room temperature. The resulting mixture was concentrated under reduced pressure. The resulting mixture was diluted with water (4 mL). The mixture was basified to pH 8 with saturated NaHCO₃ (aq.). The resulting mixture was extracted with EtOAc (3×10 mL). The combined organic layers were dried over anhydrous Na2SO4. After filtration, the filtrate was concentrated under reduced pressure. The residue was purified by silica gel column chromatography, eluted with PE/EA (3:1) to afford methyl 7-chloro-3,3-dimethyl-2H-furo[3,2-b]pyridine-5-carboxylate (110 mg, 72.58%).

### Step 7: methyl 7-ethenyl-3,3-dimethyl-2H-furo[3,2-b]pyridine-5-carboxylate

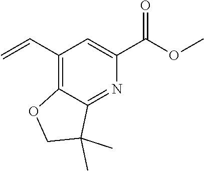 [SMILES: C=Cc1cc(C(=O)OC)nc2c1OCC2(C)C]

To a solution of methyl 7-chloro-3,3-dimethyl-2H-furo[3,2-b]pyridine-5-carboxylate (90 mg, 0.372 mmol, 1 equiv, 99.9%) and potassium ethenyltrifluoroboranuide (63 mg, 0.447 mmol, 1.20 equiv, 95%) in dioxane (4 mL, 98%) and H₂O (0.4 mL, 98%) were added K2CO3 (108 mg, 0.742 mmol, 2.00 equiv, 95%) and Pd(dppf)Cl₂ CH₂Cl₂ (35 mg, 0.041 mmol, 0.11 equiv, 95%). After stirring for overnight at 100° C. under a nitrogen atmosphere. The mixture was allowed to cool down to room temperature. The resulting mixture was concentrated under reduced pressure. The residue was purified by silica gel column chromatography, eluted with PE/EA (2:1) to afford methyl 7-ethenyl-3,3-dimethyl-2H-furo[3,2-b]pyridine-5-carboxylate (75 mg, 86.34%).

### Step 8: methyl 7-formyl-3,3-dimethyl-2H-furo[3,2-b]pyridine-5-carboxylate

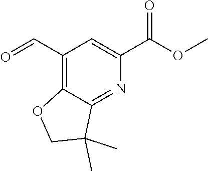 [SMILES: COC(=O)c1cc(C=O)c2c(n1)C(C)(C)CO2]

To a stirred solution of methyl 7-ethenyl-3,3-dimethyl-2H-furo[3,2-b]pyridine-5-carboxylate (70 mg, 0.300 mmol, 1 equiv, 99.9%) in dioxane (2 mL, 98%) and H₂O (2 mL, 98%) were added K₂OsO4·2H2O (14 mg, 0.036 mmol, 0.12 equiv, 95%) and NaIO₄ (133 mg, 0.591 mmol, 1.97 equiv, 95%) in portions at 0° C. The resulting mixture was stirred for 3 h at room temperature. The resulting mixture was added water (10 mL). The resulting mixture was extracted with EtOAc (3×15 mL). The combined organic layers were dried over anhydrous Na₂SO₄. After filtration, the filtrate was concentrated under reduced pressure. The crude product was used in the next step directly without further purification. This resulted in methyl 7-formyl-3,3-dimethyl-2H-furo[3,2-b]pyridine-5-carboxylate (60 mg, 53.18%).

### Step 9: methyl 3,3-dimethyl-7-{[(3S)-3-methylpiperidin-1-yl]methyl}-2H-furo[3,2-b]pyridine-5-carboxylate

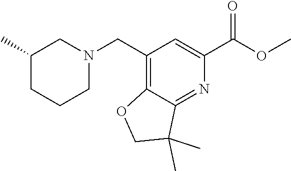

A solution of methyl 7-formyl-3,3-dimethyl-2H-furo[3,2-b]pyridine-5-carboxylate (60 mg, 0.140 mmol, 1 equiv, 54.8%) and (3S)-3-methylpiperidine hydrochloride (42 mg, 0.294 mmol, 2.10 equiv, 95%) in DCE was stirred for 2 h at 70° C. under nitrogen atmosphere. The mixture was allowed to cool down to room temperature. To the reaction mixture was added STAB (60 mg, 0.269 mmol, 1.92 equiv, 95%) in portions at room temperature. The resulting mixture was stirred for additional 2 h at 40° C. The mixture was allowed to cool down to room temperature. The resulting mixture was concentrated under reduced pressure. The residue was purified by reverse flash chromatography with the following conditions: column, C18 silica gel; mobile phase, MeCN in Water (10 mmol/L NH₄HCO₃), 45% to 50% gradient in 3 min; detector, UV 254 nm. This resulted in methyl 3,3-dimethyl-7-{[(3S)-3-methylpiperidin-1-yl]methyl}-2H-furo[3,2-b]pyridine-5-carboxylate (41 mg, 92.03%).

### Step 10: 3,3-dimethyl-7-{[(3S)-3-methylpiperidin-1-yl]methyl}-2H-furo[3,2-b]pyridine-5-carboxylic acid

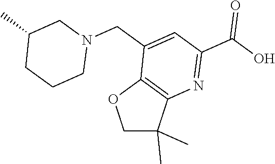 [SMILES: C[C@H]1CCCN(Cc2cc(C(=O)O)nc3c2OCC3(C)C)C1]

To a stirred solution of methyl 3,3-dimethyl-7-{[(3S)-3-methylpiperidin-1-yl]methyl}-2H-furo[3,2-b]pyridine-5-carboxylate (36 mg, 0.113 mmol, 1 equiv, 99.9%) in THF (1.5 mL, 98%) and H₂O (0.5 mL, 27.200 mmol, 240.82 equiv, 98%) was added LiOH (8 mg, 0.317 mmol, 2.81 equiv, 95%) in portions at 0° C. The resulting mixture was stirred for 1 h at room temperature. The mixture was acidified to pH 6 with HCl (aq.). The resulting mixture was concentrated under reduced pressure. The residue was purified by reverse flash chromatography with the following conditions: column, C18 silica gel; mobile phase, MeCN in Water (0.1% FA), 18% to 25% gradient in 3 min; detector, UV 220 nm. This resulted in 3,3-dimethyl-7-{[(3S)-3-methylpiperidin-1-yl]methyl}-2H-furo[3,2-b]pyridine-5-carboxylic acid (23 mg, 66.83%).

### Step 11: 3,3-dimethyl-7-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide

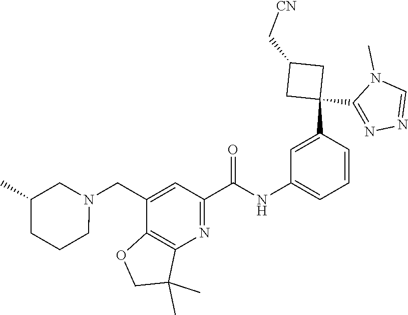 [SMILES: C[C@H]1CCCN(Cc2cc(C(=O)Nc3cccc([C@]4(c5nncn5C)C[C@@H](CC#N)C4)c3)nc3c2OCC3(C)C)C1]

A solution of 3,3-dimethyl-7-{[(3S)-3-methylpiperidin-1-yl]methyl}-2H-furo[3,2-b]pyridine-5-carboxylic acid (18 mg, 0.059 mmol, 1 equiv, 99.9%) in DMF was treated with DIEA (0.03 mL, 0.177 mmol, 3 equiv, 98%) and HATU (35 mg, 0.087 mmol, 1.48 equiv, 95%) at room temperature followed by the addition of 2-[(1s, 3s)-3-(3-aminophenyl)-3-(4-methyl-1,2,4-triazol-3-yl)cyclobutyl]acetonitrile (18 mg, 0.054 mmol, 0.91 equiv, 80%) in DMF dropwise at room temperature. The resulting mixture was stirred for overnight at 40° C. The resulting mixture was purified by Prep-HPLC with the following conditions (2 #SHIMADZU (HPLC-01)): Column, XBridge Prep OBD C18 Column, 30*150 mm, 5 μm; mobile phase, Water (10 mmol/L NH4HCO3+0.1% NH3·H2O) and ACN (42% ACN up to 70% in 8 min); This resulted in 3,3-dimethyl-7-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide (2.8 mg, 8.30%). MS: M/e 554 (M+1)⁺. ¹H NMR (400 MHz, DMSO-d6) δ 10.23 (s, 1H), 8.32 (s, 1H), 7.95 (s, 1H), 7.88-7.83 (m, 1H), 7.83-7.76 (m, 1H), 7.38 (t, J=7.9 Hz, 1H), 7.11-7.04 (m, 1H), 4.48 (s, 2H), 3.47 (s, 2H), 3.21 (s, 3H), 2.91-2.85 (m, 2H), 2.78-2.68 (m, 7H), 1.95-1.86 (m, 1H), 1.71-1.46 (m, 4H), 1.41 (s, 7H), 0.85-0.79 (m, 4H).

### Compound Cl1: 3,3-dimethyl-7-{[(3S)-3-methylpiperidin-1-yl]methyl}-N-{3-[(1r, 3r)-3-cyano-1-[(4-methyl-1,2,4-triazol-3-yl)methyl]cyclobutyl]phenyl}-2H-furo[3,2-b]pyridine-5-carboxamide

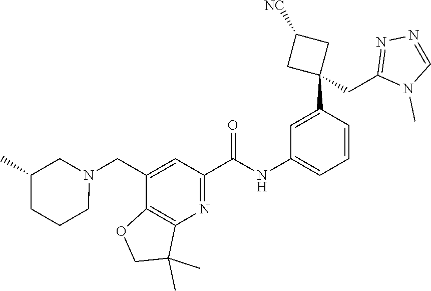 [SMILES: C[C@H]1CCCN(Cc2cc(C(=O)Nc3cccc([C@]4(Cc5nncn5C)C[C@@H](C#N)C4)c3)nc3c2OCC3(C)C)C1]

To a stirred mixture of 3,3-dimethyl-7-{[(3S)-3-methylpiperidin-1-yl]methyl}-2H-furo[3,2-b]pyridine-5-carboxylic acid (40 mg, 0.131 mmol, 1 equiv, 99.9%) and DIEA (72 mg, 0.529 mmol, 4.03 equiv, 95%) in DMF were added HATU (79 mg, 0.197 mmol, 1.50 equiv, 95%) in portions and (1r, 3r)-3-(3-aminophenyl)-3-[(4-methyl-1,2,4-triazol-3-yl)methyl]cyclobutane-1-carbonitrile (35 mg, 0.115 mmol, 0.88 equiv, 87.9%) in portions at room temperature. The resulting mixture was stirred for 6 h at 40° C. The mixture was allowed to cool down to room temperature. The crude product (60 mg) was purified by Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (35% ACN up to 65% in 8 min); Detector, This resulted in 3,3-dimethyl-7-{[(3S)-3-methylpiperidin-1-yl]methyl}-N-{3-[(1r, 3r)-3-cyano-1-[(4-methyl-1,2,4-triazol-3-yl)methyl]cyclobutyl]phenyl}-2H-furo[3,2-b]pyridine-5-carboxamide (23.8 mg, 32.51%). MS: M/e 554 (M+1)⁺. ¹H NMR (300 MHz, DMSO-d6) δ 10.12 (s, 1H), 8.18 (s, 1H), 7.96 (s, 1H), 7.76 (d, J=8.3 Hz, 1H), 7.54 (s, 1H), 7.32-7.21 (m, 1H), 6.67 (d, J=7.8 Hz, 1H), 4.49 (s, 2H), 3.48 (s, 2H), 3.30-3.19 (m, 3H), 2.85 (d, J=8.2 Hz, 4H), 2.79 (s, 3H), 2.72 (d, J=7.1 Hz, 2H), 1.99-1.83 (m, 1H), 1.60 (d, J=16.5 Hz, 4H), 1.52-1.38 (m, 7H), 0.96-0.72 (m, 4H) ppm.

### Compound C12: 3,3-dimethyl-7-{[(3S)-3-methylpiperidin-1-yl]methyl}-N-{3-[(1r, 3r)-3-cyano-1-[(4-methyl-1,2,4-triazol-3-yl)methyl]cyclobutyl]phenyl}-1H,2H-pyrrolo[3,2-b]pyridine-5-carboxamide

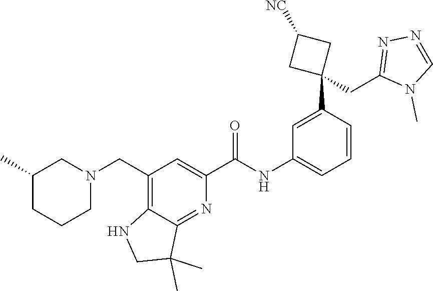 [SMILES: C[C@H]1CCCN(Cc2cc(C(=O)Nc3cccc([C@]4(Cc5nncn5C)C[C@@H](C#N)C4)c3)nc3c2NCC3(C)C)C1]

To a stirred solution of 3,3-dimethyl-7-{[(3S)-3-methylpiperidin-1-yl]methyl}-1H,2H-pyrrolo[3,2-b]pyridine-5-carboxylic acid (40 mg, 0.132 mmol, 1.06 equiv, 99.9%) in DMF were added HATU (75 mg, 0.187 mmol, 1.51 equiv, 95%) and DIEA (0.07 mL, 0.372 mmol, 3 equiv, 98%) in portions at room temperature. The reaction mixture was stirred for 30 min at 40° C. To the mixture was added (1r, 3r)-3-(3-aminophenyl)-3-[(4-methyl-1,2,4-triazol-3-yl)methyl]cyclobutane-1-carbonitrile (35 mg, 0.124 mmol, 1.00 equiv, 94.5%) in DMF dropwise at room temperature. The resulting mixture was stirred for overnight at 40° C. The mixture was allowed to cool down to room temperature. The resulting mixture was purified by Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (40% ACN up to 70% in 8 min). This resulted in 3,3-dimethyl-7-{[(3S)-3-methylpiperidin-1-yl]methyl}-N-{3-[(1r, 3r)-3-cyano-1-[(4-methyl-1,2,4-triazol-3-yl)methyl]cyclobutyl]phenyl}-1H,2H-pyrrolo[3,2-b]pyridine-5-carboxamide (18.8 mg, 27.24%). LC-MS (M+H)⁺: 553. ¹H NMR (300 MHz, DMSO-d₆) δ 9.94 (s, 1H), 8.17 (s, 1H), 7.79-7.65 (m, 2H), 7.49 (s, 1H), 7.29-7.16 (m, 1H), 6.68-6.57 (m, 1H), 6.38-6.30 (m, 1H), 3.43 (s, 2H), 3.37 (s, 2H), 3.27 (s, 3H), 2.94-2.64 (m, 9H), 1.98-1.81 (m, 1H), 1.75-1.48 (m, 5H), 1.34 (s, 6H), 0.93-0.77 (m, 4H).

### Compound C13: 3,3-dimethyl-7-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide

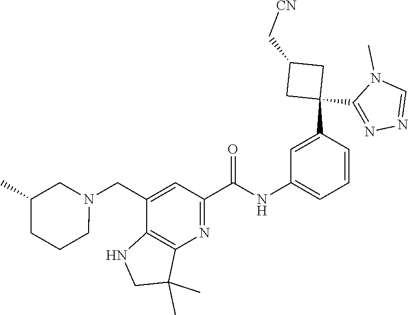 [SMILES: C[C@H]1CCCN(Cc2cc(C(=O)Nc3cccc([C@]4(c5nncn5C)C[C@@H](CC#N)C4)c3)nc3c2NCC3(C)C)C1]

### Step 1: 5-chloro-3,3-dimethyl-1H-pyrrolo[3,2-b]pyridin-2-one

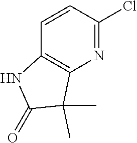

To a stirred solution of 5-chloro-1H,3H-pyrrolo[3,2-b]pyridin-2-one (3 g, 16.906 mmol, 1 equiv, 95%) and TMEDA (12.41 g, 101.436 mmol, 6 equiv, 95%) in THF was added n-BuLi (18.83 g, 67.624 mmol, 4 equiv, 23%) dropwise at −78° C. under nitrogen atmosphere. The mixture was stirred for 1 h at −78° C. under nitrogen atmosphere. To the above mixture was added MeI (10.10 g, 67.624 mmol, 4 equiv, 95%) dropwise at −78° C. The resulting mixture was stirred for additional 1 h at room temperature. The reaction was quenched by the addition of sat. NH₄Cl (aq.) (100 mL) at 0° C. The resulting mixture was extracted with EtOAc (2×100 mL), dried over anhydrous Na2SO4. After filtration, the filtrate was concentrated under reduced pressure. The residue was purified by silica gel column chromatography, eluted with PE/EA (7:1) to afford 5-chloro-3,3-dimethyl-1H-pyrrolo[3,2-b]pyridin-2-one (2 g, 52.04%). MS: M/e 197 (M+1)+.

### Step 2: 5-chloro-3,3-dimethyl-1H,2H-pyrrolo[3,2-b]pyridine

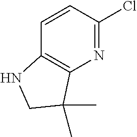

To a stirred solution of 5-chloro-3,3-dimethyl-1H-pyrrolo[3,2-b]pyridin-2-one (1 g, 4.409 mmol, 1 equiv, 86.7%) in THF was added BH3-Me2S (20.03 mL, 17.636 mmol, 4 equiv, 8.35%) dropwise at 0° C. under nitrogen atmosphere. The resulting mixture was stirred for 2 h at 75° C. under nitrogen atmosphere. The reaction was quenched by the addition of MeOH (100 mL) and NaOH (10 mL) at 0° C. The resulting mixture was concentrated under reduced pressure. The residue was purified by silica gel column chromatography, eluted with PE/EA (5:1) to afford 5-chloro-3,3-dimethyl-1H,2H-pyrrolo[3,2-b]pyridine (700 mg, 86.83%). MS: M/e 183 (M+1)+.

### Step 3: 7-bromo-5-chloro-3,3-dimethyl-1H,2H-pyrrolo[3,2-b]pyridine

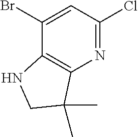

To a stirred solution of 5-chloro-3,3-dimethyl-1H,2H-pyrrolo[3,2-b]pyridine (700 mg, 3.829 mmol, 1 equiv, 99.9%) in DCM was added NBS (860.77 mg, 4.595 mmol, 1.20 equiv, 95%) at 0° C. The resulting mixture was stirred for 30 min at 0° C. under nitrogen atmosphere. The reaction was quenched by the addition of Water/Ice (50 mL) at 0° C. The resulting mixture was extracted with CH2Cl2 (2×50 mL). The combined organic layers were washed with brine (1×100 mL), dried over anhydrous Na2SO4. After filtration, the filtrate was concentrated under reduced pressure. The residue was purified by silica gel column chromatography, eluted with PE/EA (9:1) to afford 7-bromo-5-chloro-3,3-dimethyl-1H,2H-pyrrolo[3,2-b]pyridine (600 mg, 59.86%). MS: M/e 261 (M+1)+.

### Step 4: 5-chloro-7-ethenyl-3,3-dimethyl-1H,2H-pyrrolo[3,2-b]pyridine

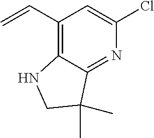

To a stirred solution of 7-bromo-5-chloro-3,3-dimethyl-1H,2H-pyrrolo[3,2-b]pyridine (600 mg, 2.292 mmol, 1 equiv, 99.9%) and 2-ethenyl-4,4,5,5-tetramethyl-1,3,2-dioxaborolane (743.10 mg, 4.584 mmol, 2 equiv, 95%) in 1,4-dioxane/H2O were added K2CO3 (1000.19 mg, 6.876 mmol, 3 equiv, 95%) and Pd(dppf)Cl2CH2Cl2 (196.51 mg, 0.229 mmol, 0.1 equiv, 95%) at room temperature. The resulting mixture was stirred for 2 h at 100° C. under nitrogen atmosphere. The mixture was allowed to cool down to room temperature. The resulting mixture was concentrated under reduced pressure. The residue was purified by silica gel column chromatography, eluted with PE/EA (9:1) to afford 5-chloro-7-ethenyl-3,3-dimethyl-1H,2H-pyrrolo[3,2-b]pyridine (300 mg, 62.66%). MS: M/e 209 (M+1)⁺

### Step 5: 1-{5-chloro-7-ethenyl-3,3-dimethyl-2H-pyrrolo[3,2-b]pyridin-1-yl}ethanone

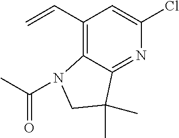 [SMILES: C=Cc1cc(Cl)nc2c1N(C(C)=O)CC2(C)C]

To a stirred solution of 5-chloro-7-ethenyl-3,3-dimethyl-1H,2H-pyrrolo[3,2-b]pyridine (300 mg, 1.436 mmol, 1 equiv, 99.9%), TEA (183.57 mg, 1.723 mmol, 1.2 equiv, 95%) and DMAP (18.47 mg, 0.144 mmol, 0.1 equiv, 95%) in DCM was added acetyl chloride (130.53 mg, 1.580 mmol, 1.1 equiv, 95%) dropwise at 0° C. under nitrogen atmosphere. The resulting mixture was stirred for overnight at room temperature under nitrogen atmosphere. The reaction was quenched by the addition of sat. NH₄Cl (aq.) (10 mL) at 0° C. The resulting mixture was extracted with CH2Cl2 (2×10 mL). The combined organic layers were washed with brine (1×20 mL), dried over anhydrous Na2SO4. After filtration, the filtrate was concentrated under reduced pressure. The residue was purified by silica gel column chromatography, eluted with PE/EA (5:1) to afford 1-{5-chloro-7-ethenyl-3,3-dimethyl-2H-pyrrolo[3,2-b]pyridin-1-yl}ethanone (200 mg, 54.43%). MS: M/e 251 (M+1)⁺.

### Step 6: 1-acetyl-7-ethenyl-3,3-dimethyl-2H-pyrrolo[3,2-b]pyridine-5-carbonitrile

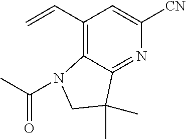 [SMILES: C=Cc1cc(C#N)nc2c1N(C(C)=O)CC2(C)C]

To a solution of 1-{5-chloro-7-ethenyl-3,3-dimethyl-2H-pyrrolo[3,2-b]pyridin-1-yl}ethanone (190 mg, 0.757 mmol, 1 equiv, 99.9%) and Zn(CN)₂ (187.14 mg, 1.514 mmol, 2 equiv, 95%) in DMA were added tBuXPhos Pd G3 (63.30 mg, 0.076 mmol, 0.1 equiv, 95%) and Zn (10.42 mg, 0.151 mmol, 0.2 equiv, 95%) at room temperature. The resulting mixture was stirred for 4 h at 130° C. under nitrogen atmosphere. The mixture was allowed to cool down to room temperature. The resulting mixture was diluted with EtOAc (10 mL). The residue was washed with brine (2×10 mL). The resulting mixture was concentrated under reduced pressure. The residue was purified by silica gel column chromatography, eluted with PE/EA (5:1) to afford 1-acetyl-7-ethenyl-3,3-dimethyl-2H-pyrrolo[3,2-b]pyridine-5-carbonitrile (90 mg, 49.22%). MS: M/e 242 (M+1)⁺.

### Step 7: 1-acetyl-7-formyl-3,3-dimethyl-2H-pyrrolo[3,2-b]pyridine-5-carbonitrile

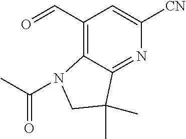 [SMILES: CC(=O)N1CC(C)(C)c2nc(C#N)cc(C=O)c21]

To a stirred solution of 1-acetyl-7-ethenyl-3,3-dimethyl-2H-pyrrolo[3,2-b]pyridine-5-carbonitrile (60 mg, 0.248 mmol, 1 equiv, 99.9%) in acetone/H2O was added NMO (45.95 mg, 0.372 mmol, 1.5 equiv, 95%) and K2OsO4·2H2O (9.63 mg, 0.025 mmol, 0.10 equiv, 95%) at room temperature. The resulting mixture was stirred for 4 h at room temperature. The reaction was quenched by the addition of Water (20 mL) at room temperature. The resulting mixture was extracted with EtOAc (2×20 mL), dried over anhydrous Na2SO4. After filtration, the filtrate was concentrated under reduced pressure. The residue was purified by silica gel column chromatography, eluted with PE/EA (1:1) to afford 1-acetyl-7-formyl-3,3-dimethyl-2H-pyrrolo[3,2-b]pyridine-5-carbonitrile (40 mg, 66.13%). MS: M/e 244 (M+1)⁺.

### Step 8: 1-acetyl-3,3-dimethyl-7-{[(3S)-3-methylpiperidin-1-yl]methyl}-2H-pyrrolo[3,2-b]pyridine-5-carbonitrile

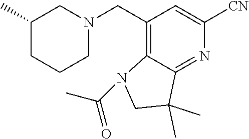 [SMILES: CC(=O)N1CC(C)(C)c2nc(C#N)cc(CN3CCC[C@H](C)C3)c21]

A solution of 1-acetyl-7-formyl-3,3-dimethyl-2H-pyrrolo[3,2-b]pyridine-5-carbonitrile (40 mg, 0.164 mmol, 1 equiv, 99.9%) and (3S)-3-methylpiperidine hydrochloride (46.91 mg, 0.328 mmol, 2 equiv, 95%) in DCE was stirred for overnight at 70° C. under nitrogen atmosphere. To the above mixture was added STAB (54.97 mg, 0.246 mmol, 1.5 equiv, 95%) at room temperature. The resulting mixture was stirred for additional 3 h at room temperature. The resulting mixture was concentrated under reduced pressure. The residue was purified by silica gel column chromatography, eluted with CH2Cl2/MeOH (10:1) to afford 1-acetyl-3,3-dimethyl-7-{[(3S)-3-methylpiperidin-1-yl]methyl}-2H-pyrrolo[3,2-b]pyridine-5-carbonitrile (30 mg, 55.89%). MS: M/e 327 (M+1)⁺.

### Step 9: 3,3-dimethyl-7-{[(3S)-3-methylpiperidin-1-yl]methyl}-1H,2H-pyrrolo[3,2-b]pyridine-5-carboxylic acid

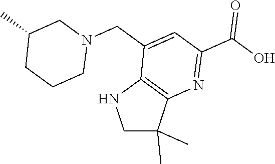 [SMILES: C[C@H]1CCCN(Cc2cc(C(=O)O)nc3c2NCC3(C)C)C1]

To a stirred solution of 1-acetyl-3,3-dimethyl-7-{[(3S)-3-methylpiperidin-1-yl]methyl}-2H-pyrrolo[3,2-b]pyridine-5-carbonitrile (25 mg, 0.077 mmol, 1 equiv, 99.9%) in H2O was added H2SO4 (1.2 mL, 22.064 mmol, 288.40 equiv, 98%) at room temperature. The resulting mixture was stirred for 4 h at 100° C. The mixture was allowed to cool down to 0° C. The mixture was neutralized to pH 5 with saturated NaHCO₃(aq.). The resulting mixture was concentrated under vacuum. The resulting mixture was diluted with MeOH (10 mL). The resulting mixture was filtered, the filter cake was washed with MeOH (2×3 mL). The filtrate was concentrated under reduced pressure. The residue was purified by reverse flash chromatography with the following conditions: column, C18 silica gel; mobile phase, MeCN in Water (0.1% HCl), 10% to 30% gradient in 10 min; detector, UV 254 nm. This resulted in 3,3-dimethyl-7-{[(3S)-3-methylpiperidin-1-yl]methyl}-1H,2H-pyrrolo[3,2-b]pyridine-5-carboxylic acid (20 mg, 86.07%). MS: M/e 304 (M+1)⁺

### Step 10: 3,3-dimethyl-7-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide

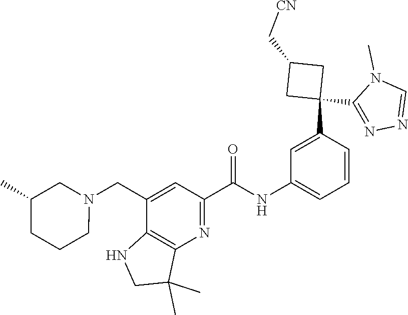 [SMILES: C[C@H]1CCCN(Cc2cc(C(=O)Nc3cccc([C@]4(c5nncn5C)C[C@@H](CC#N)C4)c3)nc3c2NCC3(C)C)C1]

To a stirred solution of 3,3-dimethyl-7-{[(3S)-3-methylpiperidin-1-yl]methyl}-1H,2H-pyrrolo[3,2-b]pyridine-5-carboxylic acid (15 mg, 0.049 mmol, 1 equiv, 99.9%) and DIEA (20.16 mg, 0.147 mmol, 3 equiv, 95%) in DMF was added HATU (29.65 mg, 0.074 mmol, 1.5 equiv, 95%) at room temperature. The resulting mixture was stirred for 10 min at room temperature. To the above mixture was added 2-[(1s, 3s)-3-(3-aminophenyl)-3-(4-methyl-1,2,4-triazol-3-yl)cyclobutyl]acetonitrile (13.22 mg, 0.049 mmol, 1 equiv, 99.9%) at room temperature. The resulting mixture was stirred for additional 4 h at room temperature. The crude product was purified by Chiral-Prep-HPLC with the following conditions (2 #SHIMADZU (HPLC-01)): Column, XBridge Prep OBD C18 Column, 30*150 mm, 5 μm; mobile phase, Water (10 mmol/L NH4HCO3+0.1% NH3·H2O) and ACN (46% ACN up to 76% in 8 min); Detector, UV product was obtained which Alpha. This resulted in 3,3-dimethyl-7-{[(3S)-3-methylpiperidin-1-yl]methyl}-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide (8.9 mg, 32.28%). ¹H NMR (300 MHz, DMSO-d6) δ 10.04 (s, 1H), 8.32 (s, 1H), 7.85 (s, 1H), 7.79-7.67 (m, 2H), 7.35 (t, J=7.9 Hz, 1H), 7.02 (d, J=7.9 Hz, 1H), 6.33 (s, 1H), 3.43 (s, 2H), 3.37 (s, 2H), 3.21 (d, J=2.0 Hz, 3H), 2.89 (s, 2H), 2.79-2.68 (m, 7H), 1.89 (t, J=11.1 Hz, 1H), 1.62 (d, J=10.4 Hz, 5H), 1.34 (d, J=2.1 Hz, 6H), 0.87-0.79 (m, 4H). MS: M/e 553 (M+1)⁺.

### Compound C14: N-(3-((1s, 3s)-3-(cyanomethyl)-1-(4-methyl-4H-1,2,4-triazol-3-yl)cyclobutyl)phenyl)-4-(((cyclohexylmethyl)amino)methyl)-7,7-dimethyl-6,7-dihydro-5H-cyclopenta[b]pyridine-2-carboxamide

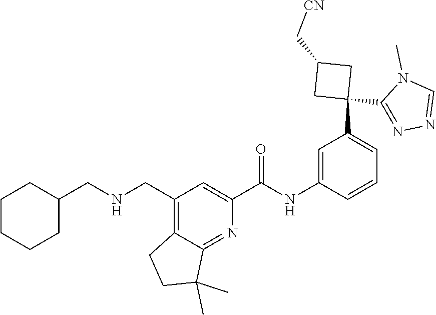 [SMILES: Cn1cnnc1[C@]1(c2cccc(NC(=O)c3cc(CNCC4CCCCC4)c4c(n3)C(C)(C)CC4)c2)C[C@H](CC#N)C1]

### Step 1: methyl 4-(((cyclohexylmethyl)amino)methyl)-7,7-dimethyl-6,7-dihydro-5H-cyclopenta[b]pyridine-2-carboxylate

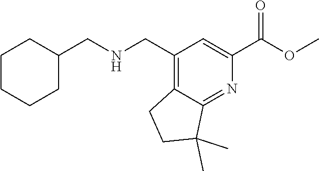 [SMILES: COC(=O)c1cc(CNCC2CCCCC2)c2c(n1)C(C)(C)CC2]

To a solution of methyl 4-formyl-7,7-dimethyl-6,7-dihydro-5H-cyclopenta[b]pyridine-2-carboxylate (200 mg, 0.857 mmol) in DCM (2 mL) was added cyclohexylmethanamine (97.06 mg, 0.857 mmol), NaBH(OAc)₃ (363.44 mg, 1.71 mmol). The resulting mixture was stirred at 20° C. for 12 hrs, then quenched with H₂O (1 mL) and extracted with DCM (3 mL, 1 mL). The combined organic layers were dried over Na₂SO₄. After filtration and concentration, the resulting residue was purified by Prep-TLC(SiO₂, PE:EA=10/1) to give the product (150 mg, 53% yield). MS: M/e 331 (M+1)⁺. ¹H NMR (400 MHz, CHLOROFORM-d) δ 7.95 (s, 1H), 3.98 (s, 3H), 3.78 (s, 2H), 2.88 (t, J=7.2 Hz, 2H), 2.47 (d, J=6.8 Hz, 2H), 2.02 (t, J=7.2 Hz, 2H), 1.85-1.64 (m, 5H), 1.54-1.43 (m, 1H), 1.35 (s, 6H), 1.33-1.11 (m, 4H), 1.00-0.87 (m, 2H) ppm.

### Step 2: methyl 4-(((tert-butoxycarbonyl)(cyclohexylmethyl)amino)methyl)-7,7-dimethyl-6,7-dihydro-5H-cyclopenta[b]pyridine-2-carboxylate

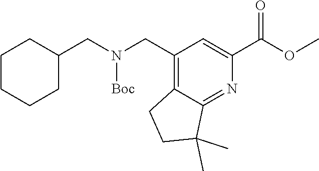 [SMILES: COC(=O)c1cc(CN(CC2CCCCC2)C(=O)OC(C)(C)C)c2c(n1)C(C)(C)CC2]

To a solution of methyl 4-(((cyclohexylmethyl)amino)methyl)-7,7-dimethyl-6,7-dihydro-5H-cyclopenta[b]pyridine-2-carboxylate (150 mg, 0.453 mmol) in THF (2 mL) was added TEA (137.79 mg, 1.36 mmol), Boc₂O (198.13 mg, 0.908 mmol). The resulting mixture was stirred at 20° C. for 2 hrs, then quenched with H₂O (1 mL) and extracted with EtOAc (3 mL, 1 mL). The combined organic layers were dried over Na₂SO₄. After filtration and concentration, the resulting residue was purified by Prep-TLC(SiO₂, PE:EA=10/1) to give the product (130 mg, 67% yield). MS: M/e 431 (M+1)⁺. ¹H NMR (400 MHz, CHLOROFORM-d) δ 7.79-7.70 (m, 1H), 4.47-4.34 (m, 2H), 3.98-3.96 (m, 3H), 3.17-3.03 (m, 2H), 2.83 (t, J=7.2 Hz, 2H), 2.09-1.98 (m, 3H), 1.75-1.65 (m, 5H), 1.56-1.46 (m, 6H), 1.35 (s, 9H), 1.23-1.15 (m, 3H), 0.99-0.89 (m, 2H) ppm.

### Step 3: 4-(((tert-butoxycarbonyl)(cyclohexylmethyl)amino)methyl)-7,7-dimethyl-6,7-dihydro-5H-cyclopenta[b]pyridine-2-carboxylic acid

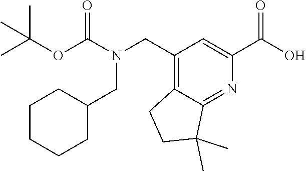 [SMILES: CC(C)(C)OC(=O)N(Cc1cc(C(=O)O)nc2c1CCC2(C)C)CC1CCCCC1]

To a solution of methyl 4-(((tert-butoxycarbonyl)(cyclohexylmethyl)amino)methyl)-7,7-dimethyl-6,7-dihydro-5H-cyclopenta[b]pyridine-2-carboxylate (130 mg, 301.92 μmol) in MeOH/THF/H₂O (0.5 mL/0.5 mL/0.5 mL) was added LiOH·H₂O (15.2 mg, 362.3 μmol). The mixture was stirred for 12 hrs at RT. The mixture was filtered and concentrated to give the crude product (130 mg, crude), which was used to the next step without purification. MS: M/e 417 (M+1)⁺; ¹H NMR (400 MHz, DMSO-d₆) δ 7.66-7.56 (m, 1H), 4.37 (s, 2H), 3.14-2.98 (m, 2H), 2.81-2.72 (m, 2H), 1.91 (t, J=7.2 Hz, 2H), 1.72-1.55 (m, 6H), 1.53-1.35 (m, 5H), 1.28-1.09 (m, 13H), 0.88 (d, J=10.0 Hz, 2H) ppm.

### Step 4: tert-butyl ((2-((3-((1s, 3s)-3-(cyanomethyl)-1-(4-methyl-4H-1,2,4-triazol-3-yl)cyclobutyl)phenyl)carbamoyl)-7,7-dimethyl-6,7-dihydro-5H-cyclopenta[b]pyridin-4-yl)methyl)(cyclohexylmethyl)carbamate

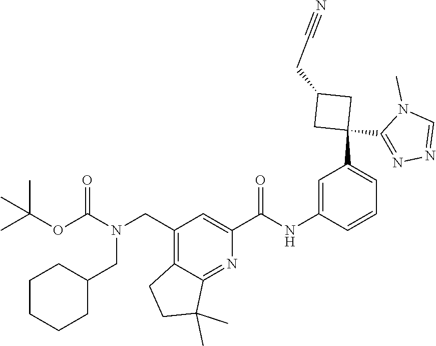 [SMILES: Cn1cnnc1[C@]1(c2cccc(NC(=O)c3cc(CN(CC4CCCCC4)C(=O)OC(C)(C)C)c4c(n3)C(C)(C)CC4)c2)C[C@H](CC#N)C1]

To a solution of 4-(((tert-butoxycarbonyl)(cyclohexylmethyl)amino)methyl)-7,7-dimethyl-6,7-dihydro-5H-cyclopenta[b]pyridine-2-carboxylic acid (130 mg, 265.27 mol) in DMF (2 mL) was added DIEA (68.57 mg, 530.54 mol) and HATU (121.04 mg, 318.33 μmol). The mixture was stirred for 5 mins at RT, then 2-((1s, 3s)-3-(3-aminophenyl)-3-(4-methyl-4H-1,2,4-triazol-3-yl)cyclobutyl)acetonitrile (70.91 mg, 265.27 μmol) was added. The mixture was stirred for 12 hrs at RT. The mixture was poured into water (1 mL) and extracted with DCM (2 mL, 1 mL). The combined organic phase was filtered and concentrated to give the residue, which was purified by Prep-HPLC (column=Waters Xbridge Prep OBD C18 (150*40 mm*10 um); mobile phase=water (NH₄HCO₃)-ACN, B %=25%-55%; 8 min) to give the product (100 mg, 56.61% yield). MS: M/e 666 (M+1), ¹H NMR (400 MHz, DMSO-d₆) δ 10.33 (s, 1H), 8.35-8.29 (m, 1H), 7.84 (br s, 1H), 7.80 (d, J=6.4 Hz, 1H), 7.74-7.66 (m, 1H), 7.38 (t, J=8.0 Hz, 1H), 7.11-7.06 (m, 1H), 4.43 (s, 2H), 3.21 (s, 3H), 3.15-3.06 (m, 2H), 2.90-2.85 (m, 4H), 2.75 (d, J=7.2 Hz, 2H), 2.70 (s, 2H), 2.01 (t, J=7.2 Hz, 2H), 1.69-1.59 (m, 6H), 1.44 (s, 4H), 1.33 (s, 9H), 1.22 (s, 4H), 1.14 (d, J=8 Hz, 2H), 0.95-0.87 (m, 2H) ppm.

### Step 5: N-(3-((1s, 3s)-3-(cyanomethyl)-1-(4-methyl-4H-1,2,4-triazol-3-yl)cyclobutyl)phenyl)-4-(((cyclohexylmethyl)amino)methyl)-7,7-dimethyl-6,7-dihydro-5H-cyclopenta[b]pyridine-2-carboxamide

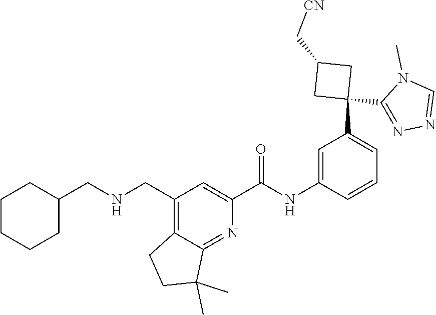 [SMILES: Cn1cnnc1[C@]1(c2cccc(NC(=O)c3cc(CNCC4CCCCC4)c4c(n3)C(C)(C)CC4)c2)C[C@H](CC#N)C1]

To a solution of tert-butyl ((2-((3-((1s, 3s)-3-(cyanomethyl)-1-(4-methyl-4H-1,2,4-triazol-3-yl)cyclobutyl)phenyl)carbamoyl)-7,7-dimethyl-6,7-dihydro-5H-cyclopenta[b]pyridin-4-yl)methyl)(cyclohexylmethyl)carbamate (50 mg, 75.09 mol) in DCM (1 mL) was added TMSI (75.13 mg, 375.54 mol) at 0° C. The mixture was poured into water (2 mL) and extracted with DCM (4 mL, 2 mL). The combined organic phase was filtered and concentrated to give the residue, which was purified by Prep-HPLC (column=Waters Xbridge Prep OBD C18 (150*40 mm*10 um); mobile phase=water (NH₄HCO₃)-ACN, B %=25%-55%; 8 min) to give the product (11.92 mg, 28.06% yield). MS: M/e 566 (M+1)⁺; ¹H NMR (400 MHz, DMSO-d₆) δ 10.32 (s, 1H), 8.34-8.28 (m, 1H), 8.00 (s, 1H), 7.86 (s, 1H), 7.79 (d, J=8.2 Hz, 1H), 7.39 (t, J=8 Hz, 1H), 7.08 (d, J=8 Hz, 1H), 3.72 (s, 2H), 3.21 (s, 3H), 2.93-2.85 (m, 4H), 2.75 (d, J=7.2 Hz, 3H), 2.71-2.68 (m, 2H), 2.36 (d, J=6.8 Hz, 2H), 1.99 (t, J=7.2 Hz, 2H), 1.77 (d, J=12 Hz, 2H), 1.66 (d, J=12.8 Hz, 3H), 1.48-1.37 (m, 1H), 1.32 (s, 6H), 1.26-1.10 (m, 4H), 0.96-0.82 (m, 2H) ppm.

### Compound C15: N-(3-((1r, 3r)-3-cyano-1-((4-methyl-4H-1,2,4-triazol-3-yl)methyl)cyclobutyl)phenyl)-4-((((3,3-difluorocyclohexyl)methyl)amino)methyl)-7,7-dimethyl-6,7-dihydro-5H-cyclopenta[b]pyridine-2-carboxamide

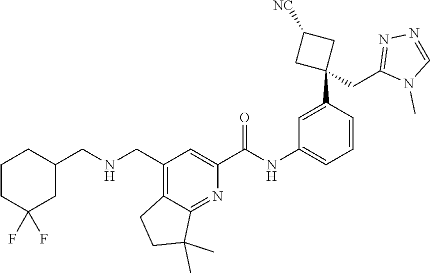 [SMILES: Cn1cnnc1C[C@]1(c2cccc(NC(=O)c3cc(CNCC4CCCC(F)(F)C4)c4c(n3)C(C)(C)CC4)c2)C[C@H](C#N)C1]

### Step 1: N-(3-((1r, 3r)-3-cyano-1-((4-methyl-4H-1,2,4-triazol-3-yl)methyl)cyclobutyl)phenyl)-4-(hydroxymethyl)-7,7-dimethyl-6,7-dihydro-5H-cyclopenta[b]pyridine-2-carboxamide

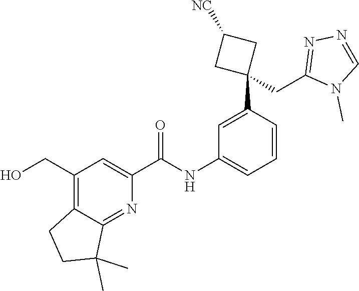 [SMILES: Cn1cnnc1C[C@]1(c2cccc(NC(=O)c3cc(CO)c4c(n3)C(C)(C)CC4)c2)C[C@H](C#N)C1]

To a mixture of lithium 4-(hydroxymethyl)-7,7-dimethyl-6,7-dihydro-5H-cyclopenta[b]pyridine-2-carboxylate (350 mg, 1.23 mmol, 80%) and (1r, 3r)-3-(3-aminophenyl)-3-((4-methyl-4H-1,2,4-triazol-3-yl)methyl)cyclobutane-1-carbonitrile (395.38 mg, 1.48 mmol) in DMF (5 mL) was added HATU (609.21 g, 1.6 mmol) and DIEA (477.88 g, 3.7 mmol). The mixture was stirred at 30° C. for 2 hrs. The mixture was poured into water (10 mL) and extracted with DCM (30 mL, 10 mL). The combined organic phase was filtered and concentrated to give the residue, which was purified by column chromatography (SiO₂, Methylene chloride/Methanol=50/1 to 10/1) to give the product (330 mg, 57% yield). MS: M/e 471 (M+1)⁺. ¹H NMR (400 MHz, DMSO-d₆) δ ppm 10.24 (s, 1H), 8.19 (s, 1H), 8.03 (s, 1H), 7.80 (d, J=7.64 Hz, 1H), 7.52 (s, 1H), 7.20-7.33 (m, 1H), 6.70 (br d, J=7.52 Hz, 1H), 5.50 (s, 1H), 4.57 (s, 2H), 3.19-3.32 (m, 4H), 2.82-2.93 (m, 5H), 1.98-2.06 (m, 2H), 1.33 (s, 6H), 1.21-1.28 (m, 3H).

### Step 2: N-(3-((1r, 3r)-3-cyano-1-((4-methyl-4H-1,2,4-triazol-3-yl)methyl)cyclobutyl)phenyl)-4-formyl-7,7-dimethyl-6,7-dihydro-5H-cyclopenta[b]pyridine-2-carboxamide

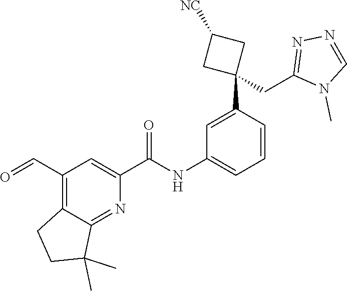 [SMILES: Cn1cnnc1C[C@]1(c2cccc(NC(=O)c3cc(C=O)c4c(n3)C(C)(C)CC4)c2)C[C@H](C#N)C1]

A mixture of N-(3-((1r, 3r)-3-cyano-1-((4-methyl-4H-1,2,4-triazol-3-yl)methyl)cyclobutyl)phenyl)-4-(hydroxymethyl)-7,7-dimethyl-6,7-dihydro-5H-cyclopenta[b]pyridine-2-carboxamide (150 mg, 318.76 mol), DMP (405.60 mg, 956.27 μmol), DCM (4 ml) was stirred at 20° C. for 12 hours. The mixture was poured into water (50 mL) and extracted with DCM (30 mL, 10 mL), adjusted to pH=7-8, The combined organic phase was filtered and washed with sat.Na₂SO₃ aqueous (30 mL, 10 mL) concentrated to give the residue, (200 mg, crude). MS: M/e 469 (M+1)⁺.

### Step 3: N-(3-((1r, 3r)-3-cyano-1-((4-methyl-4H-1,2,4-triazol-3-yl)methyl)cyclobutyl)phenyl)-4-((((3,3-difluorocyclohexyl)methyl)amino)methyl)-7,7-dimethyl-6,7-dihydro-5H-cyclopenta[b]pyridine-2-carboxamide

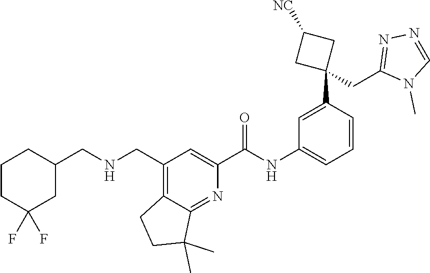 [SMILES: Cn1cnnc1C[C@]1(c2cccc(NC(=O)c3cc(CNCC4CCCC(F)(F)C4)c4c(n3)C(C)(C)CC4)c2)C[C@H](C#N)C1]

To a mixture of N-(3-((1r, 3r)-3-cyano-1-((4-methyl-4H-1,2,4-triazol-3-yl)methyl)cyclobutyl)phenyl)-4-formyl-7,7-dimethyl-6,7-dihydro-5H-cyclopenta[b]pyridine-2-carboxamide (20 mg, 42.68 mol), (3,3-difluorocyclohexyl)methanamine hydrochloride (15.85 mg, 85.37 μmol) and TEA (8.64 mg, 85.37 μmol) in DCM (0.5 mL) was added NaBH(OAc)₃ (27.14 mg, 128.05 μmol) in one portion at 20° C. The mixture was stirred at 20° C. for 12 hrs. The mixture was poured into water (1 mL) and extracted with DCM (3 mL, 1 mL). The combined organic phase was washed with brine, dried over Na₂SO₄, filtered and concentrated to give the residue, which was purified by purified by Prep-HPLC (column=Waters Xbridge Prep OBD C18 (150*40 mm*10 um); mobile phase=water (NH₄HCO₃)-ACN, B %=25%-55%; 8 min) to give the product (2.15 mg, 8.37% yield). MS: M/e 602 (M+1)⁺; ¹H NMR (400 MHz, DMSO-d₆) δ 10.23 (s, 1H), 8.20-8.15 (m, 1H), 8.04-7.98 (m, 1H), 7.78 (d, J=9.2 Hz, 1H), 7.53 (s, 1H), 7.27 (t, J=8.0 Hz, 1H), 6.69 (d, J=8.0 Hz, 1H), 3.73 (s, 3H), 3.28 (s, 2H), 3.26-3.21 (m, 2H), 3.17 (d, J=5.2 Hz, 1H), 2.92-2.83 (m, 7H), 2.81 (s, 3H), 2.00 (t, J=7.2 Hz, 3H), 1.81-1.66 (m, 4H), 1.53-1.37 (m, 3H), 1.33 (s, 6H) ppm.

### Compound C16: N-(3-((1s, 3s)-3-(cyanomethyl)-1-(4-methyl-4H-1,2,4-triazol-3-yl)cyclobutyl)phenyl)-4-((((3,3-difluorocyclopentyl)methyl)amino)methyl)-7,7-dimethyl-6,7-dihydro-5H-cyclopenta[b]pyridine-2-carboxamide

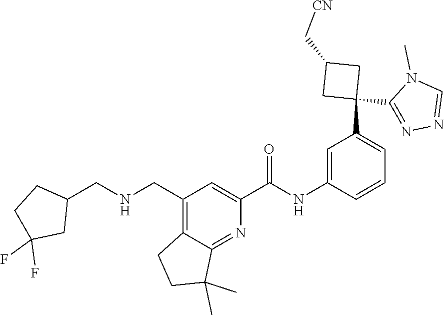 [SMILES: Cn1cnnc1[C@]1(c2cccc(NC(=O)c3cc(CNCC4CCC(F)(F)C4)c4c(n3)C(C)(C)CC4)c2)C[C@H](CC#N)C1]

### Step 1: lithium 4-(hydroxymethyl)-7,7-dimethyl-6,7-dihydro-5H-cyclopenta[b]pyridine-2-carboxylate

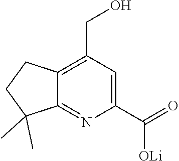

To a solution of methyl 4-(hydroxymethyl)-7,7-dimethyl-6,7-dihydro-5H-cyclopenta[b]pyridine-2-carboxylate (4 g, 17.00 mmol) in MeOH/THF/H₂O (2 mL/2 mL/2 mL) was added LiOH·H₂O (856.07 mg, 20.40 mmol). The mixture was stirred for 12 hrs at RT. The mixture was filtered and concentrated to give the crude product (3.5 g, crude), which was used to the next step without purification. MS: M/e 222 (M+1)⁺. ¹H NMR (400 MHz, DMSO-d₆) δ=7.97 (s, 1H), 4.52 (br s, 2H), 2.83-2.68 (m, 2H), 1.89 (br t, J=8.0 Hz, 2H), 1.17 (br s, 6H)

### Step 2: N-(3-((1s, 3s)-3-(cyanomethyl)-1-(4-methyl-4H-1,2,4-triazol-3-yl)cyclobutyl)phenyl)-4-(hydroxymethyl)-7,7-dimethyl-6,7-dihydro-5H-cyclopenta[b]pyridine-2-carboxamide

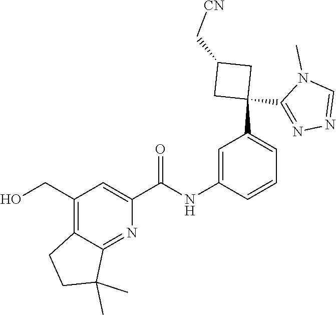 [SMILES: Cn1cnnc1[C@]1(c2cccc(NC(=O)c3cc(CO)c4c(n3)C(C)(C)CC4)c2)C[C@H](CC#N)C1]

To a solution of lithium 4-(hydroxymethyl)-7,7-dimethyl-6,7-dihydro-5H-cyclopenta[b]pyridine-2-carboxylate (254.95 mg, 1.12 mmol) in DMF (4 mL) was added DIEA (290.08 mg, 2.24 mmol) and HATU (512.03 mg, 1.35 mmol). The mixture was stirred for 5 mins at RT, then 2-((1s, 3s)-3-(3-aminophenyl)-3-(4-methyl-4H-1,2,4-triazol-3-yl)cyclobutyl)acetonitrile (300.00 mg, 1.12 mmol) was added. The mixture was stirred for 12 hrs at RT. The mixture was poured into water (10 mL) and extracted with DCM (30 mL, 10 mL). The combined organic phase was filtered and concentrated to give the residue, which was purified by column chromatography (SiO₂, EA/MeOH=100/1 to 0/1) to give the product (300 mg, 56.81% yield). MS: M/e 471 (M+1)⁺.

### Step 3: N-(3-((1s, 3s)-3-(cyanomethyl)-1-(4-methyl-4H-1,2,4-triazol-3-yl)cyclobutyl)phenyl)-4-formyl-7,7-dimethyl-6,7-dihydro-5H-cyclopenta[b]pyridine-2-carboxamide

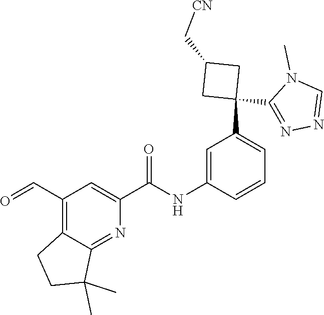 [SMILES: Cn1cnnc1[C@]1(c2cccc(NC(=O)c3cc(C=O)c4c(n3)C(C)(C)CC4)c2)C[C@H](CC#N)C1]

To a mixture of N-(3-((1s, 3s)-3-(cyanomethyl)-1-(4-methyl-4H-1,2,4-triazol-3-yl)cyclobutyl)phenyl)-4-(hydroxymethyl)-7,7-dimethyl-6,7-dihydro-5H-cyclopenta[b]pyridine-2-carboxamide (200 mg, 425.01 mol)) in DCM (2 mL) was added DMP (540.80 mg, 1.28 mmol) in one portion at 20° C. under N₂. The mixture was stirred at 20° C. and stirred for 1 hours. The residual reagent was quenched by the addition of aqueous Na₂SO₃. The aqueous solution was neutralized with aqueous NaHCO₃ and extracted with EtOAc. The organic layer was washed with brine, dried over Na₂SO₄, filtered and concentrated to give the crude product (200 mg, crude), which was used to the next step without purification. MS: M/e 469 (M+1)⁺.

### Step 4: N-(3-((1s, 3s)-3-(cyanomethyl)-1-(4-methyl-4H-1,2,4-triazol-3-yl)cyclobutyl)phenyl)-4-((((3,3-difluorocyclopentyl)methyl)amino)methyl)-7,7-dimethyl-6,7-dihydro-5H-cyclopenta[b]pyridine-2-carboxamide

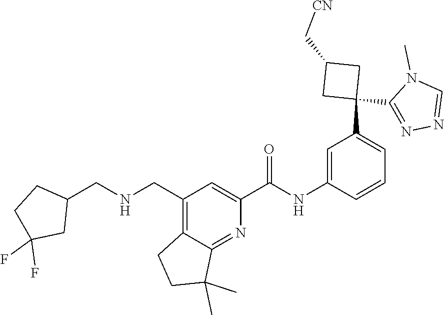 [SMILES: Cn1cnnc1[C@]1(c2cccc(NC(=O)c3cc(CNCC4CCC(F)(F)C4)c4c(n3)C(C)(C)CC4)c2)C[C@H](CC#N)C1]

To a solution of N-(3-((1s, 3s)-3-(cyanomethyl)-1-(4-methyl-4H-1,2,4-triazol-3-yl)cyclobutyl)phenyl)-4-formyl-7,7-dimethyl-6,7-dihydro-5H-cyclopenta[b]pyridine-2-carboxamide (30 mg, 64.0 umol), (3,3-difluorocyclopentyl)methanamine hydrochloride (16.5 mg, 96.0 umol) in DCM (10 mL) was added NaBH(OAc)₃ (33.9 mg, 160.1 umol). The mixture was stirred at 20° C. for 16 hrs. The reaction was concentrated. The crude was purified by Prep-HPLC(column=Waters Xbridge Prep OBD C18 150*40 mm*10 um; mobile phase=water (NH₄HCO₃)-ACN, B %=45%-65%; 8 min) to give the product to give product (2.3 mg, yield: 6%). MS: M/e 588 (M+1)⁺. 1H NMR (400 MHz, DMSO-d₆) δ=10.32 (s, 1H), 8.31 (s, 1H), 8.00 (s, 1H), 7.86 (s, 1H), 7.79 (d, J=8.0 Hz, 1H), 7.39 (t, J=8.0 Hz, 1H), 7.08 (d, J=8.0 Hz, 1H), 3.74 (s, 2H), 3.21 (s, 3H), 2.93-2.85 (m, 4H), 2.80-2.66 (m, 6H), 2.34-2.18 (m, 3H), 2.16-2.03 (m, 2H), 2.00 (t, J=7.2 Hz, 2H), 1.95-1.72 (m, 3H), 1.53-1.40 (m, 1H), 1.33 (s, 6H).

### Compound C17: N-(3-((1r,3S)-3-cyano-1-((4-methyl-4H-1,2,4-triazol-3-yl)methyl)cyclobutyl)phenyl)-7′-(((S)-3-methylpiperidin-1-yl)methyl)-1′,2′-dihydrospiro[cyclopropane-1,3′-pyrrolo[3,2-b]pyridine]-5′-carboxamide

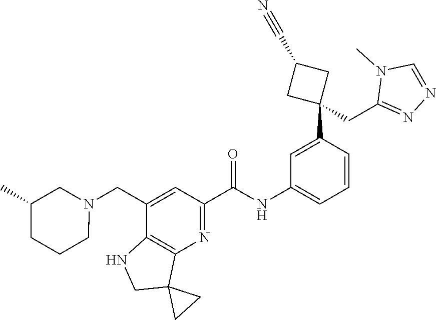 [SMILES: C[C@H]1CCCN(Cc2cc(C(=O)Nc3cccc([C@]4(Cc5nncn5C)C[C@@H](C#N)C4)c3)nc3c2NCC32CC2)C1]

### Step A: 5′-chlorospiro[cyclopropane-1,3′-pyrrolo[3,2-b]pyridin]-2′(1′H)-one

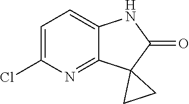 [SMILES: O=C1Nc2ccc(Cl)nc2C12CC2]

A mixture of 5-chloro-1,3-dihydro-2H-pyrrolo[3,2-b]pyridin-2-one (1 g, 10 mmol), Trifluoromethane sulfonic acid zinc (2.2 g, 5.9 mmol) and Vinyldiphenylsulfonium triflate (2.5 g, 7 mmol) in DMF (10 mL) was stirred at RT for 30 min under air atmosphere, then DBU (2.7 g, 17.7 mmol) was added and it was stirred at RT overnight. The reaction mixture was quenched by saturated NH₄Cl aq. (20 mL) (20 mL) and extracted with EtOAc (50 mL×3). The organic layer was dried, concentrated and purified by CombiFlash (PE: EtOAc=70%) to give the product (850 mg, 74%). MS: M/e 195 (M+1)⁺.

### Step B: 5′-chloro-1′,2′-dihydrospiro[cyclopropane-1,3′-pyrrolo[3,2-b]pyridine]

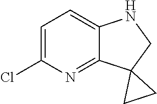 [SMILES: Clc1ccc2c(n1)C1(CC1)CN2]

To a solution of 5′-chlorospiro[cyclopropane-1,3′-pyrrolo[3,2-b]pyridin]-2′(1′H)-one (1.9 g, 9.8 mmol) in toluene (20 mL) was added Red-Al (28 mL, 3.5 M in toluene, 98 mmol) dropwise at 0° C. The reaction mixture was stirred at 50° C. overnight. The reaction was quenched by Rochelle's reagent (50 mL) and extracted with EtOAc (100 mL×2). The combined organic phases were concentrated and purified by combiflash (PE: EtOAc=70%) to give the desired product (1.5 g, 85%). MS: M/e 181 (M+1)⁺.

### Step C: 1′,2′-dihydrospiro[cyclopropane-1,3′-pyrrolo[3,2-b]pyridine]-5′-carbonitrile

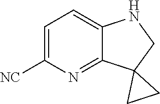 [SMILES: N#Cc1ccc2c(n1)C1(CC1)CN2]

A mixture of 5′-chloro-1′,2′-dihydrospiro[cyclopropane-1,3′-pyrrolo[3,2b]pyridine](1.5 g, 8.3 mmol), Zn(CN)₂ (1.9 g, 16.6 mmol) and tBuXPhos Pd G3 (320 mg, 0.4 mmol) in THF (30 mL) was stirred at 50° C. overnight under N₂ atmosphere. The reaction mixture was cooled to RT and filtered. The filtrates were concentrated and purified by CombiFlash (PE: EtOAc=70%) to give the desired product (1.2 g, 83%). MS: M/e 172 (M+1)⁺.

### Step D: 7-bromo-1′,2′-dihydrospiro[cyclopropane-1,3′-pyrrolo[3,2-b]pyridine]-5′-carbonitrile

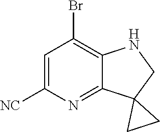 [SMILES: N#Cc1cc(Br)c2c(n1)C1(CC1)CN2]

To a solution of 1′,2′-dihydrospiro[cyclopropane-1,3′-pyrrolo[3,2-b]pyridine]-5′-carbonitrile (1.2 g, 7 mmol) in ACN (20 mL) was added NBS (1.36 g, 7.7 mmol, dissolved in ACN) dropwise at 0° C. The reaction mixture was stirred at 0° C. for 30 min. The reaction was quenched by H₂O (20 mL) and extracted with EtOAc (50 mL×2). The combined organic phases were concentrated and purified by combiflash (PE: EtOAc=85%) to give the desired product (1.5 g, 85%). MS: M/e 250 (M+1)⁺.

### Step E: (R)-7′-((3-methylpiperidin-1-yl)methyl)-1′,2′-dihydrospiro[cyclopropane-1,3′-pyrrolo[3,2-b]pyridine]-5′-carbonitrile

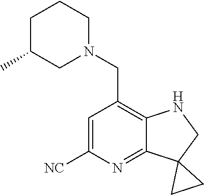 [SMILES: C[C@@H]1CCCN(Cc2cc(C#N)nc3c2NCC32CC2)C1]

A mixture of 7′-bromo-1′,2′-dihydrospiro[cyclopropane-1,3′-pyrrolo[3,2-b]pyridine]-5′-carbonitrile (120 mg, 0.5 mmol), potassium (R)-trifluoro((3-methyl piperidin-1-yl)methyl)borate (220 mg, 1 mmol), s-Phos (40 mg, 0.1 mmol), Pd(OAc)₂ (12 mg, 0.05 mmol) and Cs₂CO₃ (330 mg, 1 mmol) in Dioxane (3 mL) was stirred at 100° C. overnight under N₂ atmosphere. The reaction mixture was cooled to RT and filtered. The filtrates were concentrated and purified by CombiFlash (PE: EtOAc=20%) to give the desired product (40 mg, 30%). MS: M/e 283 (M+1)⁺.

### Step F: (R)-7′-((3-methylpiperidin-1-yl)methyl)-1′,2′-dihydrospiro[cyclopropane-1,3′-pyrrolo[3,2-b]pyridine]-5′-carboxylic acid

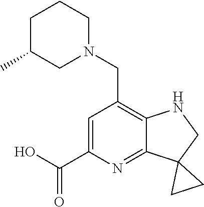 [SMILES: C[C@@H]1CCCN(Cc2cc(C(=O)O)nc3c2NCC32CC2)C1]

A mixture of (R)-7′-((3-methylpiperidin-1-yl)methyl)-1′,2′-dihydrospiro[cyclo propane-1,3′-pyrrolo[3,2-b]pyridine]-5′-carbonitrile (40 mg, 0.14 mmol), aq. NaOH (0.5 mL, ION, 5 mmol) in EtOH (1 mL) was stirred at 100° C. overnight under N₂ atmosphere. The reaction mixture was cooled to RT and acidified by 6N HCl to pH=8. The resulting mixture was concentrated and purified by CombiFlash (C18, ACN: H₂O=5%) to give the desired product (20 mg, 47%). MS: M/e 302 (M+1)⁺.

### Step G: N-(3-((1r,3S)-3-cyano-1-((4-methyl-4H-1,2,4-triazol-3-yl)methyl)cyclobutyl) phenyl)-7′-(((S)-3-methylpiperidin-1-yl)methyl)-1′,2′-dihydrospiro[cyclopropane-1,3′-pyrrolo[3,2-b]pyridine]-5′-carboxamide

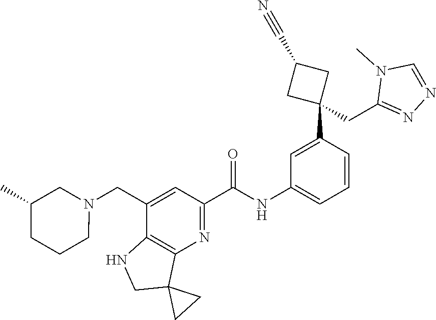 [SMILES: C[C@H]1CCCN(Cc2cc(C(=O)Nc3cccc([C@]4(Cc5nncn5C)C[C@@H](C#N)C4)c3)nc3c2NCC32CC2)C1]

To solution of (R)-7′-((3-methylpiperidin-1-yl)methyl)-1′,2′-dihydrospiro[cyclo propane-1,3′-pyrrolo[3,2-b]pyridine]-5′-carboxylic acid (20 mg, 0.07 mmol) and (1r, 3r)-3-(3-aminophenyl)-3-((4-methyl-4H-1,2,4-triazol-3-yl)methyl)cyclobutane-1-carbonitrile (30 mg, 0.1 mmol) and DIEA (40 mg, 0.3 mmol) in DMF (1 mL) were added HATU (40 mg, 0.1 mmol). The reaction mixture was stirred at RT for 2 hours. The reaction was quenched by H₂O (5 mL), extracted with DCM (10 mL×2). The organic phases were concentrated and purified by Prep-HPLC to give the desired product (1.19 mg, 3%, FA salt). ¹H NMR (400 MHz, CD₃OD) δ 8.16 (s, 1H), 7.72 (s, 1H), 7.62 (d, J=7.7 Hz, 1H), 7.42 (s, 1H), 7.27 (t, J=7.8 Hz, 1H), 6.74 (d, J=7.5 Hz, 1H), 3.85 (s, 2H), 3.82 (s, 2H), 3.44 (s, 2H), 3.14-3.02 (m, 3H), 3.01-2.97 (m, 4H), 2.79 (s, 3H), 2.40-2.37 (m, 1H), 2.11-2.07 (m, 1H), 1.79-1.67 (m, 4H), 1.42-1.37 (m, 2H), 1.09-1.03 (m, 3H), 0.94-0.93 (m, 3H). ppm. MS: M/e 551 (M+1)⁺.

### Compound C18: N-(3-((1s,3R)-3-cyano-1-((4-methyl-4H-1,2,4-triazol-3-yl)methyl)cyclobutyl)phenyl)-7,7-dimethyl-4-(((S)-3-methylpiperidin-1-yl)methyl)-6,7-dihydro-5H-cyclopenta[b]pyridine-2-carboxamide

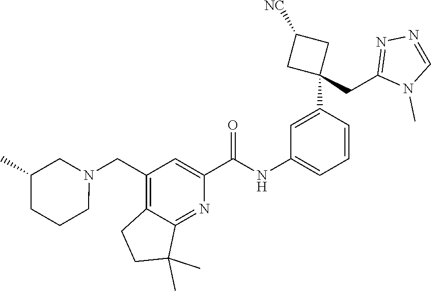 [SMILES: C[C@H]1CCCN(Cc2cc(C(=O)Nc3cccc([C@]4(Cc5nncn5C)C[C@H](C#N)C4)c3)nc3c2CCC3(C)C)C1]

To a solution of (S)-7,7-dimethyl-4-((3-methylpiperidin-1-yl)methyl)-6,7-dihydro-5H-cyclopenta[b]pyridine-2-carboxylic acid (50 mg, 165 umol) in DMF (2 mL) was added DIEA (25.6 mg, 198 umol), HATU (94.3 mg, 248 umol) and (1s, 3s)-3-(3-aminophenyl)-3-((4-methyl-4H-1,2,4-triazol-3-yl)methyl)cyclobutane-1-carbonitrile (44.2 mg, 165 umol) at 25° C. under N₂. The mixture was stirred for 1 hrs at 25° C. under N₂. The mixture was purified by Prep-HPLC (column=Waters Xbridge BEH C18 100*30 mm*10 um; mobile phase water (NH₄HCO₃)-ACN, B %=45%-65%; 8 min) to give the product (39 mg, 43% yield). MS: M/e 552 (M+1)⁺, ¹H NMR (400 MHz, DMSO-d₆) δ=10.23 (s, 1H), 8.16 (s, 1H), 7.93 (s, 1H), 7.72-7.74 (d, J=8.0 Hz, 1H), 7.39 (s, 1H), 7.23-7.27 (t, J=8.0 Hz, 1H), 6.52-6.53 (d, J=4.0 Hz, 1H), 3.73-3.77 (m, 1H), 3.50 (s, 2H), 3.23 (s, 2H), 3.01-3.06 (m, 2H), 2.89-2.93 (t, J=8.0 Hz, 2H), 2.68-2.72 (m, 5H), 2.58-2.64 (m, 2H), 1.92-2.02 (m, 3H), 1.60-1.71 (m, 4H), 1.46-1.56 (m, 1H), 1.34 (s, 6H), 0.80-0.97 (m, 4H).

### Compound C19: N-(3-((1s,3R)-3-(cyanomethyl)-1-(4-methyl-4H-1,2,4-triazol-3yl)cyclobutyl)phenyl)-7′-(((S)-3-methylpiperidin-1-yl)methyl)-1′,2′-dihydrospiro[cyclopropane-1,3′-pyrrolo[3,2-b]pyridine]-5′-carboxamide

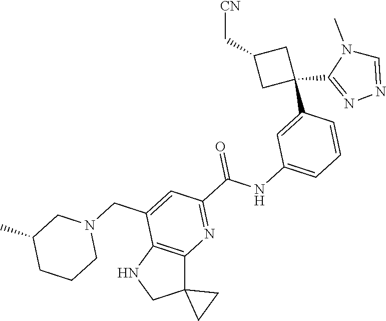 [SMILES: C[C@H]1CCCN(Cc2cc(C(=O)Nc3cccc([C@]4(c5nncn5C)C[C@@H](CC#N)C4)c3)nc3c2NCC32CC2)C1]

To solution of (R)-7′-((3-methylpiperidin-1-yl)methyl)-1′,2′-dihydrospiro[cyclo propane-1,3′-pyrrolo[3,2-b]pyridine]-5′-carboxylic acid (20 mg, 0.07 mmol) and 2-((1s, 3s)-3-(3-aminophenyl)-3-(4-methyl-4H-1,2,4-triazol-3-yl)cyclobutyl)acetonitrile (30 mg, 0.1 mmol) and DIEA (40 mg, 0.3 mmol) in DMF (1 mL) were added HATU (40 mg, 0.1 mmol). The reaction mixture was stirred at RT for 2 hours. The reaction was quenched by H₂O (5 mL), extracted with DCM (10 mL×2). The organic phases were concentrated and purified by Prep-HPLC to give the desired product (2.01 mg, 6%, FA salt). ¹H NMR (400 MHz, CD₃OD) δ 8.31 (s, 1H), 7.83 (s, 1H), 7.72 (s, 1H), 7.66 (d, J=8.3 Hz, 1H), 7.39 (t, J=7.9 Hz, 1H), 7.12 (d, J=7.9 Hz, 1H), 3.84 (s, 2H), 3.73 (s, 2H), 3.30 (s, 3H), 3.15-2.77 (m, 8H), 2.65 (d, J=6.0 Hz, 2H), 2.33-2.30 (m, 1H), 2.03-1.99 (m, 1H), 1.77-1.65 (m, 4H), 1.44-1.40 (m, 2H), 1.08-1.02 (m, 3H), 0.92 (d, J=6.3 Hz, 3H) ppm. MS: M/e 551 (M+1)⁺

### Compound C20: N-(3-((1s, 3s)-3-(cyanomethyl)-1-(4-methyl-4H-1,2,4-triazol-3-yl)cyclobutyl)phenyl)-7-((((4,4-difluorocyclohexyl)methyl)amino)methyl)-3,3-dimethyl-2,3-dihydrofuro[3,2-b]pyridine-5-carboxamide

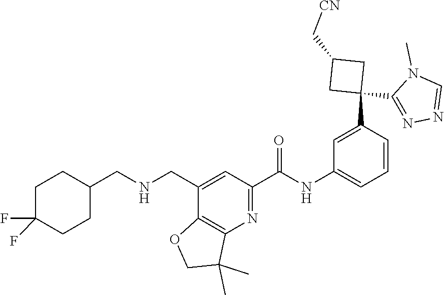 [SMILES: Cn1cnnc1[C@]1(c2cccc(NC(=O)c3cc(CNCC4CCC(F)(F)CC4)c4c(n3)C(C)(C)CO4)c2)C[C@H](CC#N)C1]

### Step 1: methyl 7-((((4,4-difluorocyclohexyl)methyl)amino)methyl)-3,3-dimethyl-2,3-dihydrofuro[3,2-b]pyridine-5-carboxylate

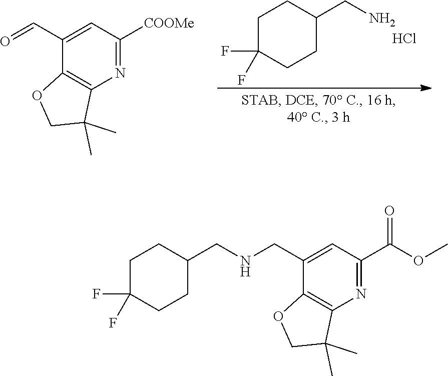 [SMILES: COC(=O)c1cc(C=O)c2c(n1)C(C)(C)CO2 ; NCC1CCC(F)(F)CC1 ; Cl ; COC(=O)c1cc(CNCC2CCC(F)(F)CC2)c2c(n1)C(C)(C)CO2]

A solution of methyl 7-formyl-3,3-dimethyl-2H-furo[3,2-b]pyridine-5-carboxylate (180 mg, 0.727 mmol, 1 equiv, 95%) and 1-(4,4-difluorocyclohexyl)methanamine hydrochloride (284.10 mg, 1.454 mmol, 2 equiv, 95%) in DCE (10 ml) was stirred for overnight at 70° C. under nitrogen atmosphere. To the above mixture was added STAB (324.34 mg, 1.454 mmol, 2 equiv, 95%) at room temperature. The resulting mixture was stirred for additional 3 hrs at 40° C. The resulting mixture was concentrated under reduced pressure. The residue was purified by silica gel column chromatography, eluted with PE/EA (1:1) to afford methyl 7-({[(4,4-difluorocyclohexyl)methyl]amino}methyl)-3,3-dimethyl-2H-furo[3,2-b]pyridine-5-carboxylate (192 mg, 64.52%). MS: M/e 369 (M+1)⁺

### Step 2: 7-((((4,4-difluorocyclohexyl)methyl)amino)methyl)-3,3-dimethyl-2,3-dihydrofuro[3,2-b]pyridine-5-carboxylic acid

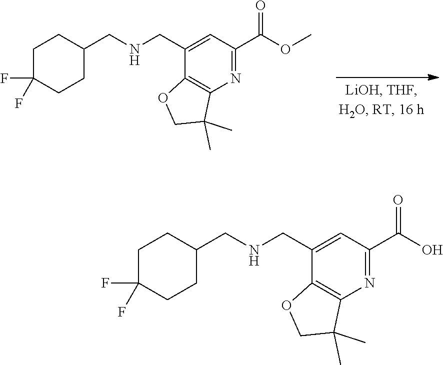 [SMILES: COC(=O)c1cc(CNCC2CCC(F)(F)CC2)c2c(n1)C(C)(C)CO2 ; CC1(C)COc2c(CNCC3CCC(F)(F)CC3)cc(C(=O)O)nc21]

A solution of methyl 7-({[(4,4-difluorocyclohexyl)methyl]amino}methyl)-3,3-dimethyl-2H-furo[3,2-b]pyridine-5-carboxylate (172 mg, 0.444 mmol, 1 equiv, 95%) and LiOH (22.36 mg, 0.888 mmol, 2 equiv, 95%) in THF (5 ml)/H₂O (3 ml) was stirred for 3 hrs at room temperature under nitrogen atmosphere. The mixture was acidified to pH=2 with HCl (aq.). The resulting mixture was concentrated under reduced pressure. The residue was purified by reverse flash chromatography with the following conditions: column, C18 silica gel; mobile phase, MeCN in Water (0.1% FA), 0% to 100% gradient in 30 min; This resulted in 7-({[(4,4-difluorocyclohexyl)methyl]amino}methyl)-3,3-dimethyl-2H-furo[3,2-b]pyridine-5-carboxylic acid (145 mg, 83.03%). MS: M/e 355 (M+1)⁺.

### Step 3: 7-(((tert-butoxycarbonyl)((4,4-difluorocyclohexyl)methyl)amino)methyl)-3,3-dimethyl-2,3-dihydrofuro[3,2-b]pyridine-5-carboxylic acid

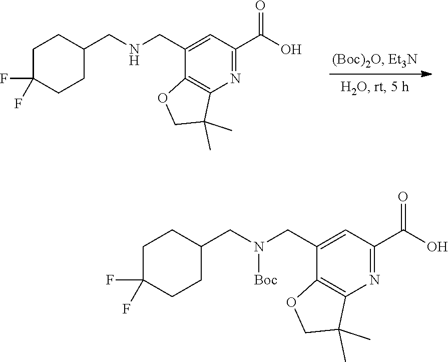 [SMILES: CC1(C)COc2c(CNCC3CCC(F)(F)CC3)cc(C(=O)O)nc21 ; CC(C)(C)OC(=O)N(Cc1cc(C(=O)O)nc2c1OCC2(C)C)CC1CCC(F)(F)CC1]

To a stirred solution of 7-({[(4,4-difluorocyclohexyl)methyl]amino}methyl)-3,3-dimethyl-2H-furo[3,2-b]pyridine-5-carboxylic acid (115 mg, 0.308 mmol, 1 equiv, 95%) and Et₃N (98.51 mg, 0.924 mmol, 3 equiv, 95%) in water (5 ml) was added Boc₂O (84.98 mg, 0.370 mmol, 1.2 equiv, 95%) dropwise at room temperature under nitrogen atmosphere. The mixture was acidified to pH=4 with HCl (aq.). The resulting mixture was concentrated under reduced pressure. The residue was purified by reverse flash chromatography with the following conditions: column, C18 silica gel; mobile phase, MeCN in Water (0.1% FA), 0% to 100% gradient in 30 min; This resulted in 7-{[(tert-butoxycarbonyl)[(4,4-difluorocyclohexyl)methyl]amino]methyl}-3,3-dimethyl-2H-furo[3,2-b]pyridine-5-carboxylic acid (120 mg, 77.08%). MS: M/e 455 (M+1)⁺

### Step 4: tert-butyl ((5-((3-((1s, 3s)-3-(cyanomethyl)-1-(4-methyl-4H-1,2,4-triazol-3-yl)cyclobutyl)phenyl)carbamoyl)-3,3-dimethyl-2,3-dihydrofuro[3,2-b]pyridin-7-yl)methyl)((4,4-difluorocyclohexyl)methyl)carbamate

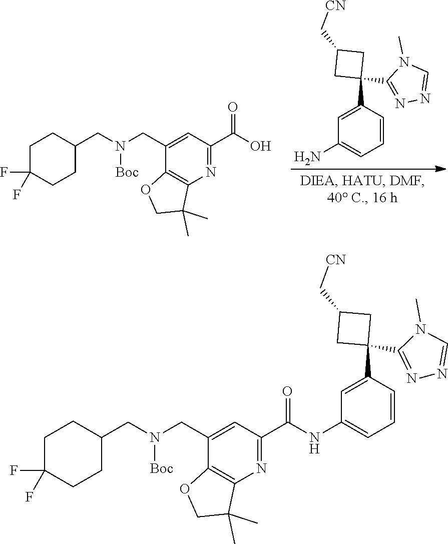 [SMILES: CC(C)(C)OC(=O)N(Cc1cc(C(=O)O)nc2c1OCC2(C)C)CC1CCC(F)(F)CC1 ; Cn1cnnc1[C@]1(c2cccc(N)c2)C[C@H](CC#N)C1 ; Cn1cnnc1[C@]1(c2cccc(NC(=O)c3cc(CN(CC4CCC(F)(F)CC4)C(=O)OC(C)(C)C)c4c(n3)C(C)(C)CO4)c2)C[C@H](CC#N)C1]

To a stirred solution of 7-{[(tert-butoxycarbonyl)[(4,4-difluorocyclohexyl)methyl]amino]methyl}-3,3-dimethyl-2H-furo[3,2-b]pyridine-5-carboxylic acid (55 mg, 0.115 mmol, 1 equiv, 95%) and DIEA (62.56 mg, 0.460 mmol, 4 equiv, 95%) in DMF (3 ml) was added HATU (69.02 mg, 0.173 mmol, 1.5 equiv, 95%) at room temperature under nitrogen atmosphere. The resulting mixture was stirred for additional 10 min at room temperature. To the above mixture was added 2-[(1s, 3s)-3-(3-aminophenyl)-3-(4-methyl-1,2,4-triazol-3-yl)cyclobutyl]acetonitrile (32.35 mg, 0.115 mmol, 1.00 equiv, 95%) at room temperature. The resulting mixture was stirred for additional 3 hrs at 40° C. The resulting mixture was concentrated under reduced pressure. The residue was purified by reverse flash chromatography with the following conditions: column, C18 silica gel; mobile phase, MeCN in Water (10 mmol/L NH₄HCO₃), 0% to 100% gradient in 30 min; This resulted in tert-butyl N-[(4,4-difluorocyclohexyl)methyl]-N-{[3,3-dimethyl-5-({3-[(1s, 3s)-3-(cyanomethyl)-1-(4-methyl-1,2,4-triazol-3-yl)cyclobutyl]phenyl}carbamoyl)-2H-furo[3,2-b]pyridin-7-yl]methyl}carbamate (68 mg, 75.64%). MS: M/e 705 (M+1)⁺

### Step 5: N-(3-((1s, 3s)-3-(cyanomethyl)-1-(4-methyl-4H-1,2,4-triazol-3-yl)cyclobutyl)phenyl)-7-((((4,4-difluorocyclohexyl)methyl)amino)methyl)-3,3-dimethyl-2,3-dihydrofuro[3,2-b]pyridine-5-carboxamide

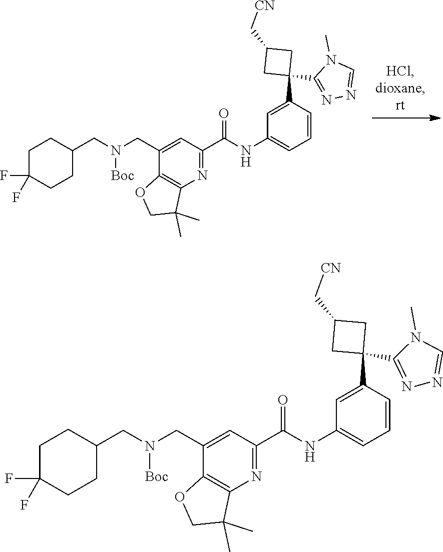 [SMILES: Cn1cnnc1[C@]1(c2cccc(NC(=O)c3cc(CN(CC4CCC(F)(F)CC4)C(=O)OC(C)(C)C)c4c(n3)C(C)(C)CO4)c2)C[C@H](CC#N)C1 ; Cn1cnnc1[C@]1(c2cccc(NC(=O)c3cc(CN(CC4CCC(F)(F)CC4)C(=O)OC(C)(C)C)c4c(n3)C(C)(C)CO4)c2)C[C@H](CC#N)C1]

A solution of tert-butyl N-[(4,4-difluorocyclohexyl)methyl]-N-{[3,3-dimethyl-5-({3-[(1s, 3s)-3-(cyanomethyl)-1-(4-methyl-1,2,4-triazol-3-yl)cyclobutyl]phenyl}carbamoyl)-2H-furo[3,2-b]pyridin-7-yl]methyl}carbamate (58 mg, 0.078 mmol, 1 equiv, 95%) and HCl (gas) in 1,4-dioxane (10 mL, 312.671 mmol, 3994.00 equiv, 95%) was stirred for 2 hrs at room temperature under nitrogen atmosphere. The resulting mixture was concentrated under reduced pressure. The mixture was basified to pH=8 with saturated NaHCO₃ (aq.). The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃+0.1% NH₃·H₂O) and ACN (36% ACN up to 66% in 8 min); This resulted in 7-({[(4,4-difluorocyclohexyl)methyl]amino}methyl)-3,3-dimethyl-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide (27.0 mg, 56.99%). MS: M/e 604 (M+1)⁺. ¹H NMR (300 MHz, DMSO-d₆) δ 10.22 (s, 1H), 8.31 (s, 1H), 8.05 (s, 1H), 7.88-7.75 (m, 2H), 7.38 (m, 1H), 7.12-7.03 (m, 1H), 4.48 (s, 2H), 3.70 (s, 2H), 3.21 (s, 3H), 2.86 (m, 2H), 2.80-2.66 (m, 5H), 2.40 (m, 2H), 2.31 (s, 1H), 2.04-1.91 (m, 2H), 1.78 (m, 4H), 1.67-1.49 (m, 1H), 1.41 (s, 6H), 1.24-1.09 (m, 2H).

### Compound C21: N-(3-((1s, 3s)-3-(cyanomethyl)-1-(4-methyl-4H-1,2,4-triazol-3-yl)cyclobutyl)phenyl)-3,3-dimethyl-7-(((1-methylcyclobutyl)amino)methyl)-2,3-dihydro-1H-pyrrolo[3,2-b]pyridine-5-carboxamido

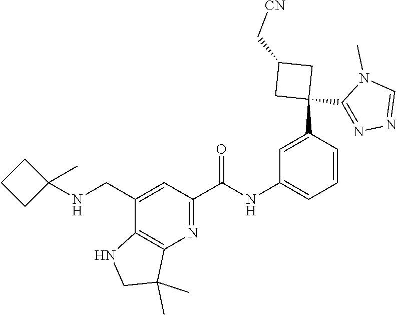 [SMILES: Cn1cnnc1[C@]1(c2cccc(NC(=O)c3cc(CNC4(C)CCC4)c4c(n3)C(C)(C)CN4)c2)C[C@H](CC#N)C1]

### Step 1: 1-acetyl-3,3-dimethyl-7-{[(1-methylcyclobutyl)amino]methyl}-2H-pyrrolo[3,2-b]pyridine-5-carbonitrile

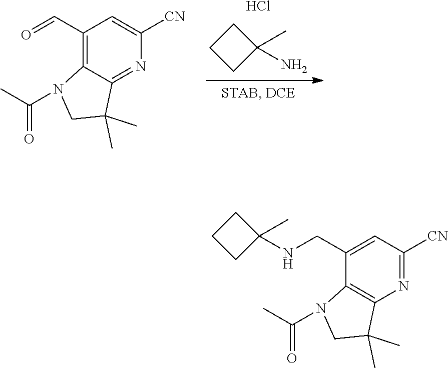

A solution of 1-acetyl-7-formyl-3,3-dimethyl-2H-pyrrolo[3,2-b]pyridine-5-carbonitrile (200 mg, 0.821 mmol, 1 equiv, 99.9%) and 1-methylcyclobutan-1-amine hydrochloride (210.28 mg, 1.642 mmol, 2 equiv, 95%) in DCE (10 mL, 100%) was stirred for 3 hrs at 70° C. under nitrogen atmosphere. To the above mixture was added STAB (274.85 mg, 1.232 mmol, 1.5 equiv, 95%) at room temperature. The resulting mixture was stirred for additional 3 hrs at 40° C. The resulting mixture was concentrated under reduced pressure. The residue was purified by silica gel column chromatography, eluted with CH₂Cl₂/MeOH (10:1) to afford 1-acetyl-3,3-dimethyl-7-{[(1-methylcyclobutyl)amino]methyl}-2H-pyrrolo[3,2-b]pyridine-5-carbonitrile (90 mg, 33.32%). MS: M/e 313 (M+1)⁺.

### Step 2: 3,3-dimethyl-7-{[(1-methylcyclobutyl)amino]methyl}-1H,2H-pyrrolo[3,2-b]pyridine-5-carboxylic acid

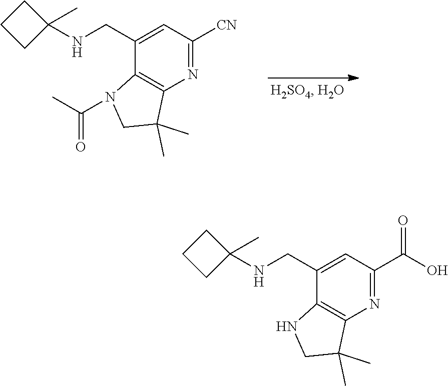 [SMILES: CC(=O)N1CC(C)(C)c2nc(C#N)cc(CNC3(C)CCC3)c21 ; CC1(NCc2cc(C(=O)O)nc3c2NCC3(C)C)CCC1]

A mixture of 1-acetyl-3,3-dimethyl-7-{[(1-methylcyclobutyl)amino]methyl}-2H-pyrrolo[3,2-b]pyridine-5-carbonitrile (80 mg, 0.243 mmol, 1 equiv, 95.0%) in H₂SO₄ (1.8 mL, 98.0%)/H₂O (1.2 mL, 100%) was stirred for 2 hrs at 100° C. under nitrogen atmosphere. The mixture was allowed to cool down to 0° C. The mixture was neutralized to pH=5 with saturated NaHCO₃ (aq.). The resulting mixture was concentrated under vacuum. The resulting mixture was diluted with MeOH (10 mL). The resulting mixture was filtered, the filter cake was washed with MeOH (3×1 mL). The filtrate was concentrated under reduced pressure. The residue was purified by reverse flash chromatography with the following conditions: column, C18 silica gel; mobile phase, MeCN in Water (0.1% HCl), 30% to 40% gradient in 4 min; This resulted in 3,3-dimethyl-7-{[(1-methylcyclobutyl)amino]methyl}-1H,2H-pyrrolo[3,2-b]pyridine-5-carboxylic acid (60 mg, 80.97%). MS: M/e 290 (M+1)⁺.

### Step 3: 3,3-dimethyl-7-{[(1-methylcyclobutyl)amino]methyl}-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide

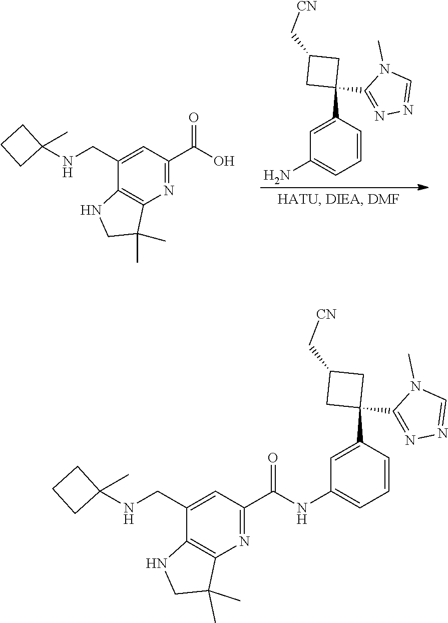 [SMILES: CC1(NCc2cc(C(=O)O)nc3c2NCC3(C)C)CCC1 ; Cn1cnnc1[C@]1(c2cccc(N)c2)C[C@H](CC#N)C1 ; Cn1cnnc1[C@]1(c2cccc(NC(=O)c3cc(CNC4(C)CCC4)c4c(n3)C(C)(C)CN4)c2)C[C@H](CC#N)C1]

To a stirred solution of 3,3-dimethyl-7-{[(1-methylcyclobutyl)amino]methyl}-1H,2H-pyrrolo[3,2-b]pyridine-5-carboxylic acid (50 mg, 0.164 mmol, 1 equiv, 95.0%) and DIEA (67.00 mg, 0.492 mmol, 3 equiv, 95%) in DMF (1 mL, 100%) was added HATU (98.55 mg, 0.246 mmol, 1.5 equiv, 95%) at room temperature. The resulting mixture was stirred for 10 min at room temperature. To the above mixture was added 2-[(1s, 3s)-3-(3-aminophenyl)-3-(4-methyl-1,2,4-triazol-3-yl)cyclobutyl]acetonitrile (43.93 mg, 0.164 mmol, 1 equiv, 99.9%) at room temperature. The resulting mixture was stirred for additional 2 hrs at room temperature. The crude product was purified by Chiral-Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (40% ACN up to 70% in 8 min); product was obtained which Alpha. This resulted in 3,3-dimethyl-7-{[(1-methylcyclobutyl)amino]methyl}-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide (33.3 mg, 37.58%). MS: M/e 539 (M+1)⁺. 1H NMR (400 MHz, DMSO-d₆) δ 10.04 (s, 1H), 8.31 (s, 1H), 7.88-7.82 (m, 2H), 7.80-7.73 (m, 1H), 7.39-7.31 (m, 1H), 7.05-6.98 (m, 1H), 6.36 (s, 1H), 3.54-3.41 (m, 4H), 3.21 (s, 3H), 2.88 (d, J=4.8 Hz, 2H), 2.80-2.67 (m, 5H), 2.33 (s, 1H), 2.04-1.92 (m, 2H), 1.77-1.60 (m, 4H), 1.34 (s, 6H), 1.24 (s, 3H).

### Compound C22: 3,3-dimethyl-7-({[(1-methylcyclopentyl)methyl]amino}methyl)-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide

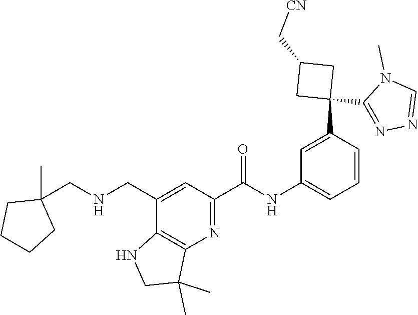 [SMILES: Cn1cnnc1[C@]1(c2cccc(NC(=O)c3cc(CNCC4(C)CCCC4)c4c(n3)C(C)(C)CN4)c2)C[C@H](CC#N)C1]

### Step 1: 7-formyl-3,3-dimethyl-1H,2H-pyrrolo[3,2-b]pyridine-5-carboxylic acid

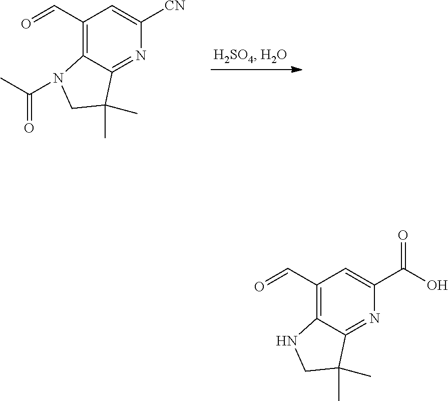 [SMILES: CC(=O)N1CC(C)(C)c2nc(C#N)cc(C=O)c21 ; CC1(C)CNc2c(C=O)cc(C(=O)O)nc21]

A solution of 1-acetyl-7-formyl-3,3-dimethyl-2H-pyrrolo[3,2-b]pyridine-5-carbonitrile (60 mg, 0.246 mmol, 1 equiv, 99.9%) in H₂SO₄ (1 mL, 11.257 mmol, 45.69 equiv, 60%) was stirred for 3 hrs at 100° C. under nitrogen atmosphere. The mixture was allowed to cool down to 0° C. The mixture was neutralized to pH=3 with NaOH (2N). The resulting mixture was concentrated under vacuum. The resulting mixture was diluted with MeOH (5 mL). The resulting mixture was filtered, the filter cake was washed with MeOH (3×5 mL). The filtrate was concentrated under reduced pressure. The residue was purified by reverse flash chromatography with the following conditions: column, C18 silica gel; mobile phase, MeCN in Water (0.1% TFA), 25% to 30% gradient in 3 min; This resulted in 7-formyl-3,3-dimethyl-1H,2H-pyrrolo[3,2-b]pyridine-5-carboxylic acid (50 mg, 92.05%). MS: M/e 221 (M+1)⁺.

### Step 2: 7-formyl-3,3-dimethyl-N-{3-[(1s, 3s)-3-(cyanomethyl)-1-(4-methyl-1,2,4-triazol-3-yl)cyclobutyl]phenyl}-1H, 2H-pyrrolo[3,2-b]pyridine-5-carboxamide

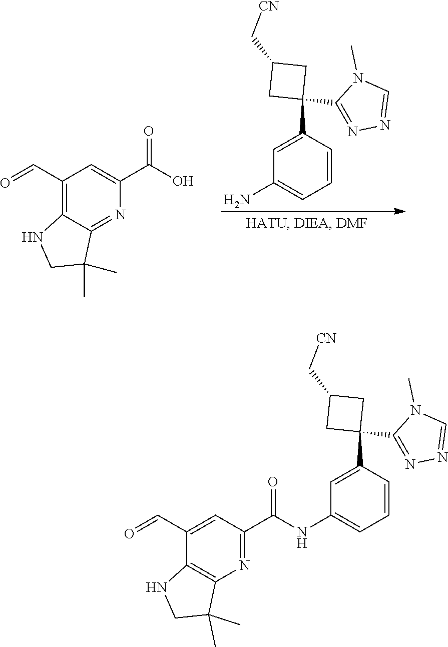 [SMILES: CC1(C)CNc2c(C=O)cc(C(=O)O)nc21 ; Cn1cnnc1[C@]1(c2cccc(N)c2)C[C@H](CC#N)C1 ; Cn1cnnc1[C@]1(c2cccc(NC(=O)c3cc(C=O)c4c(n3)C(C)(C)CN4)c2)C[C@H](CC#N)C1]

A solution of 7-formyl-3,3-dimethyl-1H,2H-pyrrolo[3,2-b]pyridine-5-carboxylic acid (50 mg, 0.227 mmol, 1.21 equiv, 99.9%) in DMF (3 mL) was treated with HATU (112 mg, 0.280 mmol, 1.50 equiv, 95%) at room temperature followed by the addition of DIEA (0.10 mL, 0.561 mmol, 3 equiv, 98%) dropwise at room temperature. The resulting mixture was stirred for 0.5 h at room temperature. To the mixture was added 2-[(1s, 3s)-3-(3-aminophenyl)-3-(4-methyl-1,2,4-triazol-3-yl)cyclobutyl]acetonitrile (50 mg, 0.187 mmol, 1.00 equiv, 99.9%) in portions at room temperature. The resulting mixture was stirred for additional 2 hrs at room temperature. The residue was purified by reversed-phase flash chromatography with the following conditions: column, C18 silica gel; mobile phase, MeCN in Water (10 mmol/L NH₄HCO₃), 40% to 50% gradient in 3 min; This resulted in 7-formyl-3,3-dimethyl-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide (50 mg, 56.93%). MS: M/e 470 (M+1)⁺

### Step 3: 3,3-dimethyl-7-({[(1-methylcyclopentyl)methyl]amino}methyl)-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide

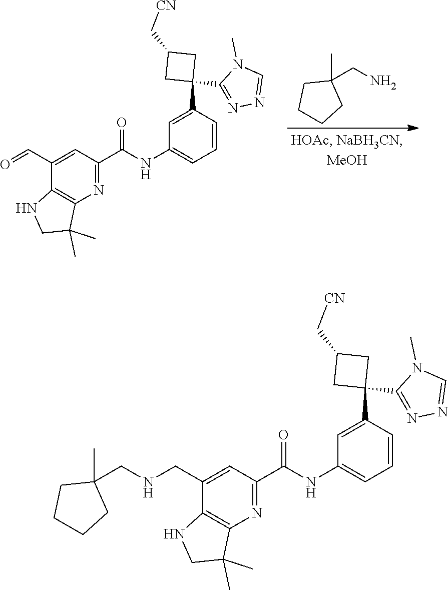

A solution of 7-formyl-3,3-dimethyl-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide (60 mg, 0.125 mmol, 1 equiv, 97.6%), HOAc (0.02 mL, 0.342 mmol, 2.74 equiv, 98%) and 1-(1-methylcyclopentyl)methanamine (30 mg, 0.252 mmol, 2.02 equiv, 95%) in MeOH (3 mL, 98%) was stirred for 30 min at room temperature. To the mixture was added NaBH₃CN (11 mg, 0.166 mmol, 1.33 equiv, 95%) in portions at 0° C. The resulting mixture was stirred for additional 1 h at room temperature. The reaction mixture was purified by Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (40% ACN up to 70% in 8 min); This resulted in 3,3-dimethyl-7-({[(1-methylcyclopentyl)methyl]amino}methyl)-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide (32.2 mg, 44.42%). MS: M/e 567 (M+1)⁺. ¹H NMR (300 MHz, DMSO-d₆) δ 10.06 (s, 1H), 8.32 (s, 1H), 7.88-7.82 (m, 1H), 7.82-7.72 (m, 2H), 7.35 (t, J=7.9 Hz, 1H), 7.02 (d, J=7.8 Hz, 1H), 6.38 (s, 1H), 3.64 (s, 2H), 3.42 (s, 2H), 3.21 (s, 3H), 2.88 (s, 2H), 2.79-2.67 (m, 5H), 2.30 (s, 2H), 2.08 (s, 1H), 1.63-1.40 (m, 6H), 1.34 (s, 6H), 1.30-1.17 (m, 2H), 0.98 (s, 3H).

### Compound C23: N-(3-((1s, 3s)-3-(cyanomethyl)-1-(4-methyl-4H-1,2,4-triazol-3-yl)cyclobutyl)phenyl)-3,3-dimethyl-7-(((1-methylcyclobutyl)amino)methyl)-2,3-dihydrofuro[3,2-b]pyridine-5-carboxamide

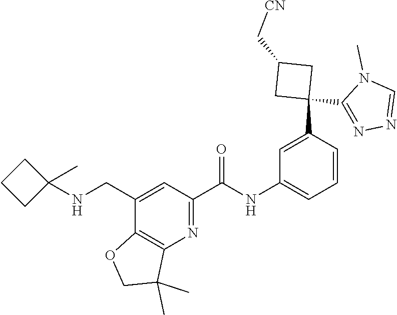 [SMILES: Cn1cnnc1[C@]1(c2cccc(NC(=O)c3cc(CNC4(C)CCC4)c4c(n3)C(C)(C)CO4)c2)C[C@H](CC#N)C1]

### Step 1: methyl 3,3-dimethyl-7-(((1-methylcyclobutyl)amino)methyl)-2,3-dihydrofuro[3,2-b]pyridine-5-carboxylate

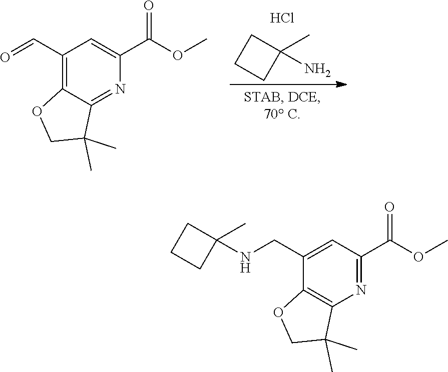

A solution of methyl 7-formyl-3,3-dimethyl-2H-furo[3,2-b]pyridine-5-carboxylate (235 mg, 0.949 mmol, 1 equiv, 95%) and 1-methylcyclobutan-1-amine hydrochloride (242.97 mg, 1.898 mmol, 2 equiv, 95%) in DCE (5 ml) was stirred for overnight at 70° C. under nitrogen atmosphere. To the above mixture was added STAB (423.45 mg, 1.898 mmol, 2 equiv, 95%) at room temperature. The resulting mixture was stirred for additional 2 hrs at 40° C. The resulting mixture was concentrated under reduced pressure. The residue was purified by silica gel column chromatography, eluted with PE/EA (1:1) to afford methyl 3,3-dimethyl-7-{[(1-methylcyclobutyl)amino]methyl}-2H-furo[3,2-b]pyridine-5-carboxylate (230 mg, 71.66%). MS: M/e 305 (M+1)⁺

### Step 2: 3,3-dimethyl-7-(((1-methylcyclobutyl)amino)methyl)-2,3-dihydrofuro[3,2-b]pyridine-5-carboxylic acid

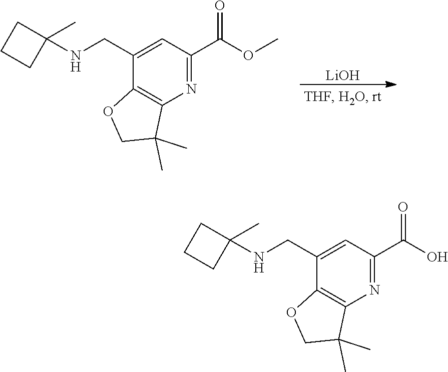 [SMILES: COC(=O)c1cc(CNC2(C)CCC2)c2c(n1)C(C)(C)CO2 ; "LiOH" ; CC1(NCc2cc(C(=O)O)nc3c2OCC3(C)C)CCC1]

A solution of methyl 3,3-dimethyl-7-{[(1-methylcyclobutyl)amino]methyl}-2H-furo[3,2-b]pyridine-5-carboxylate (210 mg, 0.655 mmol, 1 equiv, 95%) and LiOH (33.05 mg, 1.310 mmol, 2 equiv, 95%) in THF (5 ml)/H₂O (2 ml) was stirred for 3 hrs at room temperature under nitrogen atmosphere. The mixture was acidified to pH=5 with HCl (aq.). The resulting mixture was concentrated under reduced pressure. The residue was purified by reversed-phase flash chromatography with the following conditions: column, C18 silica gel; mobile phase, MeCN in Water (0.1% TFA), 0% to 100% gradient in 30 min; This resulted in 3,3-dimethyl-7-{[(1-methylcyclobutyl)amino]methyl}-2H-furo[3,2-b]pyridine-5-carboxylic acid (160 mg, 75.67%). MS: M/e 291 (M+1)⁺

### Step 3: N-(3-((1s, 3s)-3-(cyanomethyl)-1-(4-methyl-4H-1,2,4-triazol-3-yl)cyclobutyl)phenyl)-3,3-dimethyl-7-(((1-methylcyclobutyl)amino)methyl)-2,3-dihydrofuro[3,2-b]pyridine-5-carboxamide

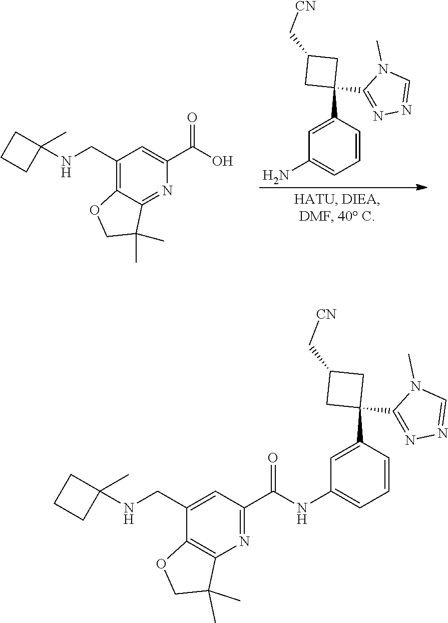 [SMILES: CC1(NCc2cc(C(=O)O)nc3c2OCC3(C)C)CCC1 ; Cn1cnnc1[C@]1(c2cccc(N)c2)C[C@H](CC#N)C1 ; Cn1cnnc1[C@]1(c2cccc(NC(=O)c3cc(CNC4(C)CCC4)c4c(n3)C(C)(C)CO4)c2)C[C@H](CC#N)C1]

To a stirred solution of 3,3-dimethyl-7-{[(1-methylcyclobutyl)amino]methyl}-2H-furo[3,2-b]pyridine-5-carboxylic acid (70 mg, 0.229 mmol, 1 equiv, 95%) and DIEA (124.63 mg, 0.916 mmol, 4 equiv, 95%) in DMF (5 ml) was added HATU (137.50 mg, 0.344 mmol, 1.5 equiv, 95%) at room temperature under nitrogen atmosphere. The resulting mixture was stirred for additional 10 min at room temperature. To the above mixture was added 2-[(1s, 3s)-3-(3-aminophenyl)-3-(4-methyl-1,2,4-triazol-3-yl)cyclobutyl]acetonitrile (64.45 mg, 0.229 mmol, 1 equiv, 95%) at room temperature. The resulting mixture was stirred for additional 16 hrs at 40° C. The crude product (was purified by Chiral-Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (38% ACN up to 68% in 8 min); This resulted in 3,3-dimethyl-7-{[(1-methylcyclobutyl)amino]methyl}-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide (41.8 mg, 33.11%). MS: M/e 540 (M+1)⁺. ¹H NMR (300 MHz, DMSO-d₆) δ 10.21 (s, 1H), 8.32 (s, 1H), 8.10 (s, 1H), 7.89-7.76 (m, 2H), 7.38 (m, 1H), 7.11-7.02 (m, 1H), 4.49 (s, 2H), 3.63 (s, 2H), 3.21 (s, 3H), 2.93-2.66 (m, 7H), 2.39 (s, 1H), 1.95 (m, 2H), 1.77-1.60 (m, 4H), 1.41 (s, 6H), 1.22 (s, 3H).

### Compound C24: N-(3-((1s, 3s)-3-(cyanomethyl)-1-(4-methyl-4H-1,2,4-triazol-3-yl)cyclobutyl)phenyl)-7,7-dimethyl-4(((1methylcyclopropyl)amino)methyl)-6,7-dihydro-5H-cyclopenta[b]pyridine-2-carboxamide

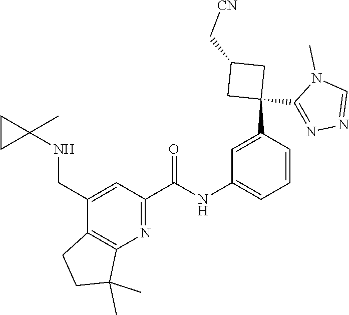 [SMILES: Cn1cnnc1[C@]1(c2cccc(NC(=O)c3cc(CNC4(C)CC4)c4c(n3)C(C)(C)CC4)c2)C[C@H](CC#N)C1]

To a solution of N-(3-((1s, 3s)-3-(cyanomethyl)-1-(4-methyl-4H-1,2,4-triazol-3-yl)cyclobutyl)phenyl)-4-formyl-7,7-dimethyl-6,7-dihydro 5Hcyclopenta[b]pyridine-2-carboxamide (35 mg, 74.70 umol), 1-methylcyclopropan-1-amine hydrochloride (12.05 mg, 112.05 umol), Et₃N (11.34 mg, 112.05 umol) in DCE (2 mL) was added NaBH(OAc)₃ (94.99 mg, 448.18 umol) was added and the mixture was stirred at 50° C. for 16 hrs. The mixture was concentrated, the residue was purified by Prep-HPLC (column=Phenomenex C18 80*40 mm*3 um; mobile phase=water (NH₄HCO₃)—CAN; B %=30%-60%, 8 min) to give the product (10.28 mg, 26% yield). MS: M/e 524 (M+1)⁺; ¹H NMR (400 MHz, DMSO-d₆) δ ppm 10.31 (s, 1H), 8.31 (s, 1H), 7.95 (s, 1H), 7.86 (s, 1H), 7.79 (d, J=7.88 Hz, 1H), 7.38 (t, J=7.94 Hz, 1H), 7.08 (d, J=7.88 Hz, 1H), 3.76 (s, 2H), 3.21 (s, 3H), 2.65-2.95 (m, 10H), 1.94-2.01 (m, 2H), 1.32 (s, 6H), 1.25 (s, 3H), 0.48-0.53 (m, 2H), 0.28-0.34 (m, 2H).

### Compound C25: N-(3-((1s, 3s)-3-(cyanomethyl)-1-(4-methyl-4H-1,2,4-triazol-3-yl)cyclobutyl)phenyl)-3,3-dimethyl-7-((((1-methylcyclobutyl)methyl)amino)methyl)-2,3-dihydrofuro[3,2-b]pyridine-5-carboxamide

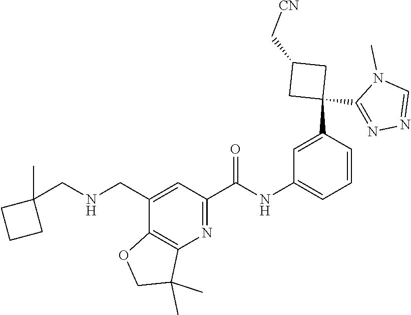 [SMILES: Cn1cnnc1[C@]1(c2cccc(NC(=O)c3cc(CNCC4(C)CCC4)c4c(n3)C(C)(C)CO4)c2)C[C@H](CC#N)C1]

### Step 1: 7-formyl-3,3-dimethyl-2,3-dihydrofuro[3,2-b]pyridine-5-carboxylic acid

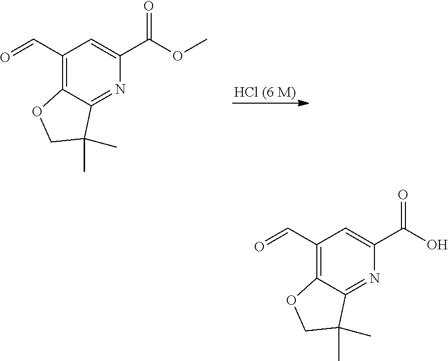 [SMILES: COC(=O)c1cc(C=O)c2c(n1)C(C)(C)CO2 ; CC1(C)COc2c(C=O)cc(C(=O)O)nc21]

Into a 20 mL sealed tube were added methyl 7-formyl-3,3-dimethyl-2H-furo[3,2-b]pyridine-5-carboxylate (200 mg, 0.765 mmol, 1 equiv, 90%) and HCl (2 mL). The resulting mixture was stirred for 4 hrs at 90° C. The mixture was allowed to cool down to room temperature. The residue was purified by reversed-phase flash chromatography with the following conditions: column, C18 silica gel; mobile phase, MeCN in Water (0.1% FA), 0% to 95% gradient in 20 min; The crude product (150 mg) was purified by Prep-HPLC with the following conditions (2 #SHIMADZU (HPLC-01)): Column, Xselect CSH C18 OBD Column 30*150 mm 5 um, n; mobile phase, undefined and undefined (15% undefined up to 45% in 8 min); This resulted in 7-formyl-3,3-dimethyl-2H-furo[3,2-b]pyridine-5-carboxylic acid (105 mg, 56.57%). MS: M/e 221 (M+1)⁺

### Step 2: N-(3-((1s, 3s)-3-(cyanomethyl)-1-(4-methyl-4H-1,2,4-triazol-3-yl)cyclobutyl)phenyl)-7-formyl-3,3-dimethyl-2,3-dihydrofuro[3,2-b]pyridine-5-carboxamide

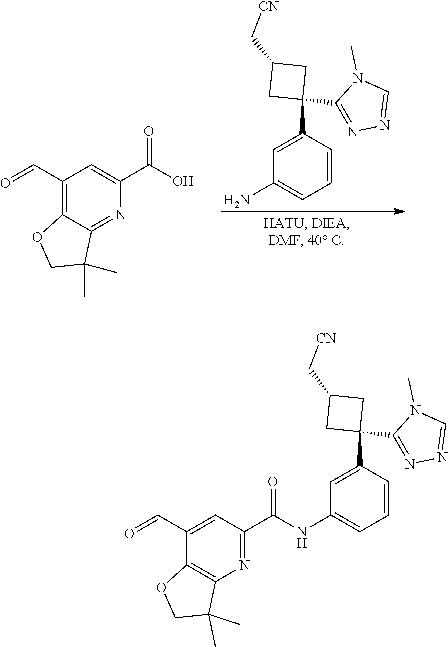 [SMILES: CC1(C)COc2c(C=O)cc(C(=O)O)nc21 ; Cn1cnnc1[C@]1(c2cccc(N)c2)C[C@H](CC#N)C1 ; Cn1cnnc1[C@]1(c2cccc(NC(=O)c3cc(C=O)c4c(n3)C(C)(C)CO4)c2)C[C@H](CC#N)C1]

To a stirred mixture of 7-formyl-3,3-dimethyl-2H-furo[3,2-b]pyridine-5-carboxylic acid (90 mg, 0.371 mmol, 1 equiv, 91.2%) and DIEA (152 mg, 1.117 mmol, 3.01 equiv, 95%) in DMF (3 mL) was added HATU (223 mg, 0.557 mmol, 1.50 equiv, 95%) in portions at 0° C. and 2-[(1s, 3s)-3-(3-aminophenyl)-3-(4-methyl-1,2,4-triazol-3-yl)cyclobutyl]acetonitrile (99 mg, 0.370 mmol, 1.00 equiv, 99.9%) in DMF (1 mL) at 0° C. The resulting mixture was stirred for 1 h at room temperature. The residue was purified by reversed-phase flash chromatography with the following conditions: column, C18 silica gel; mobile phase, MeCN in Water (10 mmol/L NH₄HCO₃), 0% to 95% gradient in 30 min; This resulted in 7-formyl-3,3-dimethyl-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide (120 mg, 65.36%). MS: M/e 471 (M+1)⁺

### Step 3: N-(3-((1s, 3s)-3-(cyanomethyl)-1-(4-methyl-4H-1,2,4-triazol-3-yl)cyclobutyl)phenyl)-3,3-dimethyl-7-((((1-methylcyclobutyl)methyl)amino)methyl)-2,3-dihydrofuro[3,2-b]pyridine-5-carboxamide

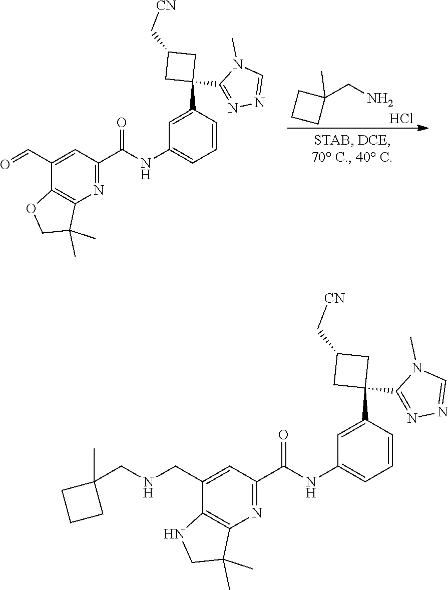

A solution of 7-formyl-3,3-dimethyl-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide (41 mg, 0.083 mmol, 1 equiv, 95%) and 1-(1-methylcyclobutyl)methanamine hydrochloride (23.64 mg, 0.166 mmol, 2 equiv, 95%) in DCE (3 ml) was stirred for overnight at 70° C. under nitrogen atmosphere. To the above mixture was added STAB (36.93 mg, 0.166 mmol, 2 equiv, 95%) at 25° C. The resulting mixture was stirred for additional 3 h at 40° C. The crude product was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃+0.1% NH₃·H₂O) and ACN (37% ACN up to 65% in 8 min); This resulted in 3,3-dimethyl-7-({[(1-methylcyclobutyl)methyl]amino}methyl)-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide (16.0 mg, 34.56%). MS: M/e 554 (M+1)⁺. ¹H NMR (300 MHz, DMSO-d₆) δ 10.24 (s, 1H), 8.32 (s, 1H), 8.11 (s, 1H), 7.88-7.77 (m, 2H), 7.38 (m, 1H), 7.09 (m, 1H), 4.50 (s, 2H), 3.83 (s, 2H), 3.22 (s, 3H), 2.93-2.85 (m, 2H), 2.80-2.67 (m, 5H), 2.53 (s, 2H), 1.94-1.80 (m, 3H), 1.78-1.70 (m, 1H), 1.69-1.54 (m, 2H), 1.42 (s, 6H), 1.14 (s, 3H).

### Compound C26: N-(3-((1s,3R)-3-(cyanomethyl)-1-(4-methyl-4H-1,2,4-triazol-3-yl)cyclobutyl)phenyl)-7-((((S)-1-cyclopropylethyl)amino)methyl)-3,3-dimethyl-2,3-dihydrofuro[3,2-b]pyridine-5-carboxamide

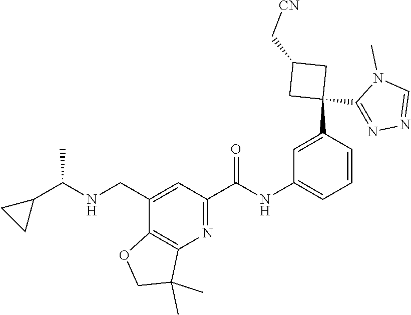 [SMILES: C[C@H](NCc1cc(C(=O)Nc2cccc([C@]3(c4nncn4C)C[C@@H](CC#N)C3)c2)nc2c1OCC2(C)C)C1CC1]

To a stirred solution of 7-formyl-3,3-dimethyl-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide (60 mg, 0.121 mmol, 1 equiv, 95%) and (1S)-1-cyclopropylethanamine hydrochloride (31.01 mg, 0.242 mmol, 2 equiv, 95%) in MeOH (5 ml) was added tetrakis(propan-2-yloxy)titanium (144.97 mg, 0.484 mmol, 4 equiv, 95%) at room temperature under nitrogen atmosphere. The resulting mixture was stirred for additional 1 h at room temperature. To the above mixture was added NaBH₃CN (16.03 mg, 0.242 mmol, 2 equiv, 95%) at room temperature. The resulting mixture was stirred for additional 1 h at room temperature. The crude product (was purified by Chiral-Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃+0.1% NH₃·H₂O) and ACN (30% ACN up to 58% in 8 min); This resulted in 7-({[(1S)-1-cyclopropylethyl]amino}methyl)-3,3-dimethyl-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide (15.8 mg, 23.32%). MS: M/e 540 (M+1)⁺. ¹H NMR (300 MHz, DMSO-d₆) δ 10.22 (s, 1H), 8.32 (s, 1H), 8.08 (s, 1H), 7.89-7.76 (m, 2H), 7.38 (m, 1H), 7.07 (m, 1H), 4.49 (s, 2H), 3.89-3.71 (m, 2H), 3.21 (s, 3H), 2.89 (s, 2H), 2.79-2.67 (m, 5H), 2.32 (s, 1H), 1.84 (m, 1H), 1.41 (m, 6H), 1.09 (m, 3H), 0.65 (m, 1H), 0.50-0.26 (m, 2H), 0.23-0.09 (m, 1H), 0.02 (m, 1H).

### Compound C27: 7-({[(1S)-1-cyclopropylethyl]amino}methyl)-3,3-dimethyl-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide

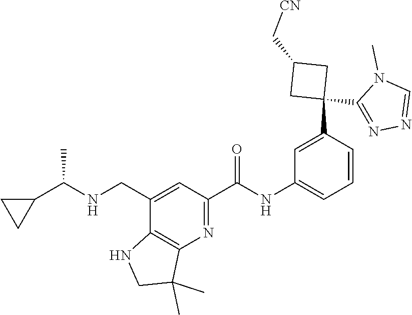 [SMILES: C[C@H](NCc1cc(C(=O)Nc2cccc([C@]3(c4nncn4C)C[C@@H](CC#N)C3)c2)nc2c1NCC2(C)C)C1CC1]

The compound C27 was synthesized starting from the corresponding starting materials according the similar procedures described as those of Compound C22 to give the product (29.6 mg, 47.15%). MS: M/e 540 (M+1)⁺. ¹H NMR (300 MHz, DMSO-d₆) δ 10.04 (s, 1H), 8.31 (s, 1H), 7.88-7.78 (m, 2H), 7.81-7.72 (m, 1H), 7.35 (t, J=7.9 Hz, 1H), 7.02 (d, J=8.0, 1.1 Hz, 1H), 6.40 (s, 1H), 3.69 (d, J=2.6 Hz, 2H), 3.43 (d, J=1.3 Hz, 2H), 3.21 (s, 3H), 2.92-2.79 (m, 2H), 2.79-2.66 (m, 5H), 2.28 (s, 1H), 1.90-1.75 (m, 1H), 1.34 (d, J=2.5 Hz, 6H), 1.09 (d, J=6.3 Hz, 3H), 0.77-0.60 (m, 1H), 0.51-0.26 (m, 2H), 0.26-0.12 (m, 1H), 0.09-−0.05 (m, 1H).

### Compound C28: 7-({[(1S)-1-cyclobutylethyl]amino}methyl)-3,3-dimethyl-N-{3-[(1s, 3s)-3-(cyanomethyl)-1-(4-methyl-1,2,4-triazol-3-yl)cyclobutyl]phenyl}-2H-furo[3,2-b]pyridine-5-carboxamide

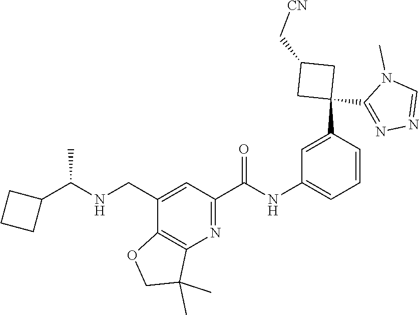 [SMILES: C[C@H](NCc1cc(C(=O)Nc2cccc([C@]3(c4nncn4C)C[C@@H](CC#N)C3)c2)nc2c1OCC2(C)C)C1CCC1]

The compound C28 was synthesized starting from the corresponding starting materials according the similar procedures described as those of Compound C26 to give the product (34.3 mg, 47.94%). MS: M/e 554 (M+1)⁺. ¹H NMR (300 MHz, DMSO-d₆) δ 10.22 (s, 1H), 8.32 (s, 1H), 8.07 (s, 1H), 7.87-7.76 (m, 2H), 7.44-7.32 (m, 1H), 7.11-7.02 (m, 1H), 4.48 (s, 2H), 3.81-3.62 (m, 2H), 3.21 (s, 3H), 2.96-2.82 (m, 2H), 2.80-2.65 (m, 5H), 2.47-2.36 (m, 1H), 2.30-2.08 (m, 1H), 2.05-1.55 (m, 7H), 1.41 (d, J=1.8 Hz, 6H), 0.88 (d, J=6.2 Hz, 3H).

### Compound C29: 4-(((cyclobutylmethyl)amino)methyl)-7,7-dimethyl-N-(5-((1s, 3s)-3-methyl-1-(4-methyl-4H-1,2,4-triazol-3-yl)cyclobutyl)pyridin-3-yl)-6,7-dihydro-5H-cyclopenta[b]pyridine-2-carboxamide

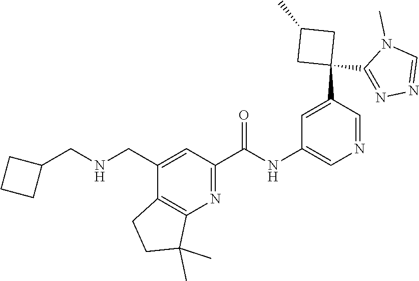 [SMILES: Cn1cnnc1[C@]1(c2cncc(NC(=O)c3cc(CNCC4CCC4)c4c(n3)C(C)(C)CC4)c2)C[C@H](C)C1]

### Step 1: methyl 4-(bromomethyl)-7,7-dimethyl-6,7-dihydro-5H-cyclopenta[b]pyridine-2-carboxylate

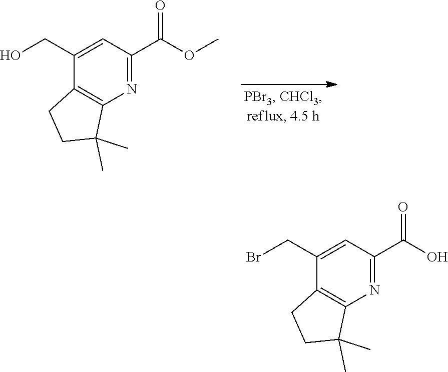 [SMILES: COC(=O)c1cc(CO)c2c(n1)C(C)(C)CC2 ; CC1(C)CCc2c(CBr)cc(C(=O)O)nc21]

A solution of methyl 4-(hydroxymethyl)-7,7-dimethyl-5H,6H-cyclopenta[b]pyridine-2-carboxylate (150 mg, 0.606 mmol, 1 equiv, 95%) and PBr₃ (86.29 mg, 0.303 mmol, 0.5 equiv, 95%) in CHCl₃(5 ml) was stirred for 4 hrs at 70° C. under nitrogen atmosphere. The resulting mixture was concentrated under reduced pressure. The residue was purified by reversed-phase flash chromatography with the following conditions: column, C18 silica gel; mobile phase, MeCN in Water (0.1% TFA), 0% to 100% gradient in 30 min; This resulted in methyl 4-(bromomethyl)-7,7-dimethyl-5H,6H-cyclopenta[b]pyridine-2-carboxylate (130 mg, 71.98%). MS: M/e 298 (M+1)⁺

### Step 2: methyl 4-(((cyclobutylmethyl)amino)methyl)-7,7-dimethyl-6,7-dihydro-5H-cyclopenta[b]pyridine-2-carboxylate

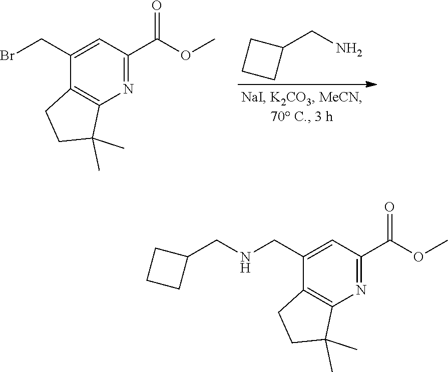

To a stirred solution of methyl 4-(bromomethyl)-7,7-dimethyl-5H,6H-cyclopenta[b]pyridine-2-carboxylate (120 mg, 0.382 mmol, 1 equiv, 95%) and 1-cyclobutylmethanamine (68.54 mg, 0.764 mmol, 2 equiv, 95%) in acetonitrile (5 ml) were added K₂CO₃ (111.24 mg, 0.764 mmol, 2 equiv, 95%) and NaI (60.32 mg, 0.382 mmol, 1 equiv, 95%) at room temperature under nitrogen atmosphere. The residue was purified by reversed-phase flash chromatography with the following conditions: column, C18 silica gel; mobile phase, MeCN in Water (0.1% NH₃·H₂O), 0% to 100% gradient in 30 min; This resulted in methyl 4-([(cyclobutylmethyl)amino]methyl-7,7-dimethyl-5H,6H-cyclopenta[b]pyridine-2-carboxylate (68 mg, 52.93%). MS: M/e 303 (M+1)⁺

### Step 3: methyl 4-(((tert-butoxycarbonyl)(cyclobutylmethyl)amino)methyl)-7,7-dimethyl-6,7-dihydro-5H-cyclopenta[b]pyridine-2-carboxylate

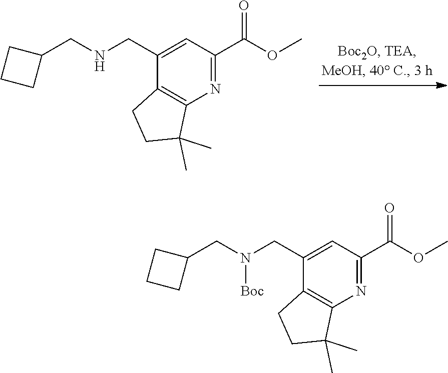 [SMILES: COC(=O)c1cc(CNCC2CCC2)c2c(n1)C(C)(C)CC2 ; COC(=O)c1cc(CN(CC2CCC2)C(=O)OC(C)(C)C)c2c(n1)C(C)(C)CC2]

To a stirred solution of methyl 4-([(cyclobutylmethyl)amino]methyl-7,7-dimethyl-5H,6H-cyclopenta[b]pyridine-2-carboxylate (63 mg, 0.198 mmol, 1 equiv, 95%) and Et₃N (63.24 mg, 0.594 mmol, 3 equiv, 95%) in DCM (3 ml) was added Boc₂O (68.20 mg, 0.297 mmol, 1.5 equiv, 95%) at room temperature under nitrogen atmosphere. The resulting mixture was stirred for additional 3 hrs at room temperature. The residue was purified by reversed-phase flash chromatography with the following conditions: column, C18 silica gel; mobile phase, MeCN in Water (0.1% TFA), 0% to 100% gradient in 30 min; This resulted in methyl 4-([(tert-butoxycarbonyl)(cyclobutylmethyl)amino]methyl-7,7-dimethyl-5H,6H-cyclopenta[b]pyridine-2-carboxylate (55 mg, 62.14%). MS: M/e 403 (M+1)⁺

### Step 4: 4-(((tert-butoxycarbonyl)(cyclobutylmethyl)amino)methyl)-7,7-dimethyl-6,7-dihydro-5H-cyclopenta[b]pyridine-2-carboxylic acid

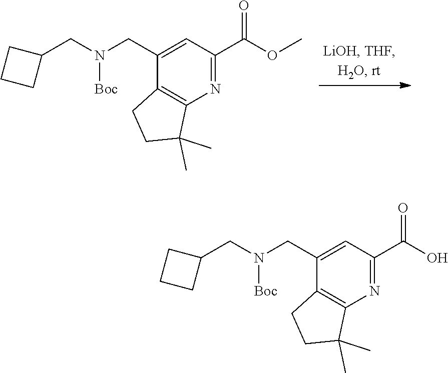 [SMILES: COC(=O)c1cc(CN(CC2CCC2)C(=O)OC(C)(C)C)c2c(n1)C(C)(C)CC2 ; CC(C)(C)OC(=O)N(Cc1cc(C(=O)O)nc2c1CCC2(C)C)CC1CCC1]

A solution of methyl 4-([(tert-butoxycarbonyl)(cyclobutylmethyl)amino]methyl-7,7-dimethyl-5H,6H-cyclopenta[b]pyridine-2-carboxylate (50 mg, 0.118 mmol, 1 equiv, 95%) and LiOH·H₂O (20.85 mg, 0.472 mmol, 4 equiv, 95%) in THF/H₂O (5 ml) was stirred for 2 hrs at room temperature under nitrogen atmosphere. The resulting mixture was concentrated under reduced pressure. The mixture was acidified to pH=2 with HCl (aq.). The residue was purified by reversed-phase flash chromatography with the following conditions: column, C18 silica gel; mobile phase, MeCN in Water (0.1% TFA), 0% to 100% gradient in 30 min; This resulted in 4-([(tert-butoxycarbonyl)(cyclobutylmethyl)amino]methyl-7,7-dimethyl-5H,6H-cyclopenta[b]pyridine-2-carboxylic acid (36 mg, 78.53%). MS: M/e 389 (M+1)⁺

### Step 5: tert-butyl (cyclobutylmethyl)((7,7-dimethyl-2-((5-((1s, 3s)-3-methyl-1-(4-methyl-4H-1,2,4-triazol-3-yl)cyclobutyl)pyridin-3-yl)carbamoyl)-6,7-dihydro-5H-cyclopenta[b]pyridin-4-yl)methyl)carbamate

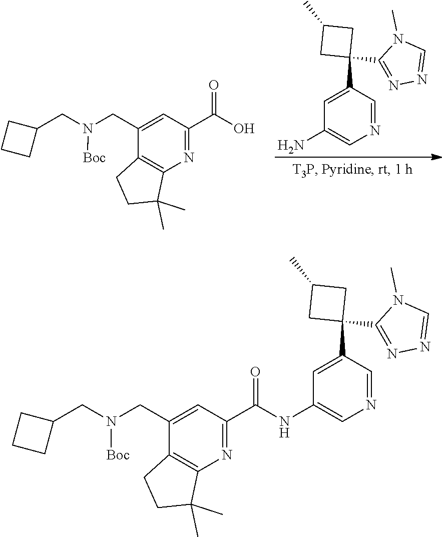 [SMILES: CC(C)(C)OC(=O)N(Cc1cc(C(=O)O)nc2c1CCC2(C)C)CC1CCC1 ; Cn1cnnc1[C@]1(c2cncc(N)c2)C[C@H](C)C1 ; Cn1cnnc1[C@]1(c2cncc(NC(=O)c3cc(CN(CC4CCC4)C(=O)OC(C)(C)C)c4c(n3)C(C)(C)CC4)c2)C[C@H](C)C1]

To a stirred solution of 4-([(tert-butoxycarbonyl)(cyclobutylmethyl)amino]methyl-7,7-dimethyl-5H,6H-cyclopenta[b]pyridine-2-carboxylic acid (31 mg, 0.076 mmol, 1 equiv, 95%) and 5-[(1r, 3s)-3-methyl-1-(4-methyl-1,2,4-triazol-3-yl)cyclobutyl]pyridin-3-amine (23.30 mg, 0.091 mmol, 1.2 equiv, 95%) in pyridine (2 ml) was added tetrakis(propan-2-yloxy)titanium (226.78 mg, 0.760 mmol, 10 equiv, 95%) dropwise at 0° C. under nitrogen atmosphere. The resulting mixture was stirred for additional 1 h at room temperature. The resulting mixture was concentrated under reduced pressure. The residue was purified by reversed-phase flash chromatography with the following conditions: column, C18 silica gel; mobile phase, MeCN in Water (0.1% NH3·H2O), 00% to 100% gradient in 30 min; This resulted in tert-butyl N-(cyclobutylmethyl)-N-([7,7-dimethyl-2-((5-[(1r, 3s)-3-methyl-1-(4-methyl-1,2,4-triazol-3-yl)cyclobutyl]pyridin-3-ylcarbamoyl)-5H,6H-cyclopenta[b]pyridin-4-yl]methylcarbamate (35 mg, 75.22%). MS: M/e 614 (M+1)⁺

### Step 6: 4-(((cyclobutylmethyl)amino)methyl)-7,7-dimethyl-N-(5-((1s, 3s)-3-methyl-1-(4-methyl-4H-1,2,4-triazol-3-yl)cyclobutyl)pyridin-3-yl)-6,7-dihydro-5H-cyclopenta[b]pyridine-2-carboxamide

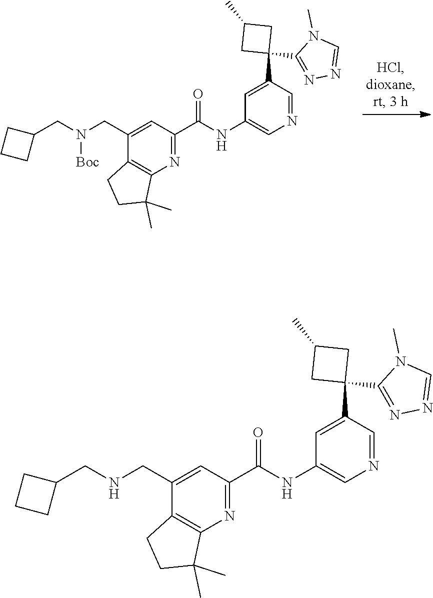 [SMILES: Cn1cnnc1[C@]1(c2cncc(NC(=O)c3cc(CN(CC4CCC4)C(=O)OC(C)(C)C)c4c(n3)C(C)(C)CC4)c2)C[C@H](C)C1 ; Cn1cnnc1[C@]1(c2cncc(NC(=O)c3cc(CNCC4CCC4)c4c(n3)C(C)(C)CC4)c2)C[C@H](C)C1]

A solution of tert-butyl N-(cyclobutylmethyl)-N-([7,7-dimethyl-2-((5-[(1r, 3s)-3-methyl-1-(4-methyl-1,2,4-triazol-3-yl)cyclobutyl]pyridin-3-ylcarbamoyl)-5H,6H-cyclopenta[b]pyridin-4-yl]methylcarbamate (35 mg, 0.054 mmol, 1 equiv, 95%) and HCl (gas) in 1,4-dioxane (5 mL, 164.564 mmol, 3037.91 equiv) was stirred for 2 hrs at room temperature under nitrogen atmosphere. The crude product was purified by Chiral-Prep-HPLC with the following conditions (2 #SHIMADZU (HPLC-01)): Column: XBridge Shield RP18 OBD Column 30*150 mm, 5 m; mobile phase, 10 mmol NH₄HCO₃+0.1% NH₃H₂O and ACN (45% ACN up to 70% in 9 min); This resulted in 4-([(cyclobutylmethyl)amino]methyl-7,7-dimethyl-N-(5-[(1r, 3s)-3-methyl-1-(4-methyl-1,2,4-triazol-3-yl)cyclobutyl]pyridin-3-yl-5H,6H-cyclopenta[b]pyridine-2-carboxamide (25.6 mg, 91.91%). MS: M/e 514 (M+1)⁺. ¹H NMR (400 MHz, DMSO-d₆) δ 10.50 (s, 1H), 9.01 (m, 1H), 8.36-8.30 (m, 2H), 8.21 (m, 1H), 8.01 (s, 1H), 3.73 (s, 2H), 3.23 (s, 3H), 2.94-2.85 (m, 4H), 2.62-2.52 (m, 5H), 2.43 (m, 1H), 2.19 (s, 1H), 2.07-1.94 (m, 4H), 1.91-1.74 (m, 2H), 1.72-1.59 (m, 2H), 1.34 (s, 6H), 1.10 (m, 3H).

### Compound C30: N-(3-((1s, 3s)-3-(cyanomethyl)-1-(4-methyl-4H-1,2,4-triazol-3-yl)cyclobutyl)phenyl)-7-(((cyclobutylmethyl)amino)methyl)-3,3-dimethyl-2,3-dihydro-1H-pyrrolo[3,2-b]pyridine-5-carboxamide

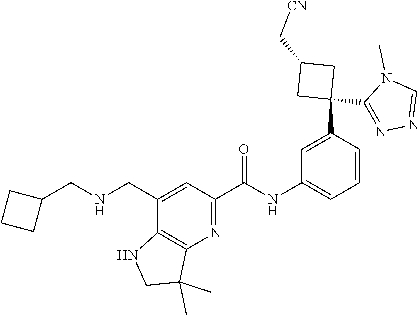 [SMILES: Cn1cnnc1[C@]1(c2cccc(NC(=O)c3cc(CNCC4CCC4)c4c(n3)C(C)(C)CN4)c2)C[C@H](CC#N)C1]

The compound C266 was synthesized starting from the corresponding starting materials according to the similar procedures described as those of Compound C22 to give the product (33.8 mg, 51.58%). MS: M/e 539 (M+1)⁺; ¹H NMR (300 MHz, DMSO-d₆) δ 10.05 (s, 1H), 8.32 (s, 1H), 7.87-7.72 (m, 3H), 7.35 (m, 1H), 7.02 (m, 1H), 6.37 (s, 1H), 3.60 (s, 2H), 3.42 (s, 2H), 3.21 (s, 3H), 2.92-2.66 (m, 9H), 2.47-2.34 (m, 1H), 2.09-1.93 (m, 2H), 1.83 (m, 2H), 1.73-1.58 (m, 2H), 1.34 (s, 6H).

### Compound C31: N-(3-((1s, 3s)-3-(cyanomethyl)-1-(4-methyl-4H-1,2,4-triazol-3-yl)cyclobutyl)phenyl)-7′-(((1-methylcyclobutyl)amino)methyl)-1′,2′-dihydrospiro[cyclopropane-1,3′-pyrrolo[3,2-b]pyridine]-5′-carboxamide

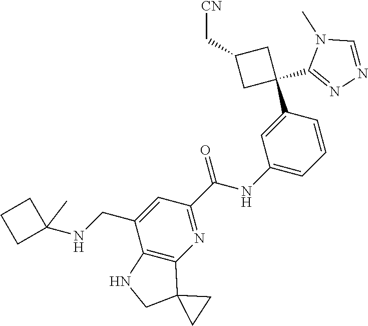 [SMILES: Cn1cnnc1[C@]1(c2cccc(NC(=O)c3cc(CNC4(C)CCC4)c4c(n3)C3(CC3)CN4)c2)C[C@H](CC#N)C1]

### Step A: 7-formyl-1′,2′-dihydrospiro[cyclopropane-1,3′-pyrrolo[3,2-b]pyridine]-5′-carbonitrile

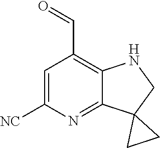 [SMILES: N#Cc1cc(C=O)c2c(n1)C1(CC1)CN2]

A mixture of 7′-bromo-1′,2′-dihydrospiro[cyclopropane-1,3′-pyrrolo[3,2b]pyridine]-5′-carbonitrile (750 mg, 3 mmol), N-ForMylsaccharin (950 mg, 4.5 mmol),

Pd(OAc)₂ (67 mg, 0.3 mmol), xant-phos (350 mg, 0.6 mmol), Na₂CO₃ (640 mg, 6 mmol) and Triethylsilane (520 mg, 4.5 mmol) in DMF (8 mL) was stirred at 80° C. for 3 hours under N₂ atmosphere. The reaction mixture was cooled to rt and quenched by H₂O (10 mL) and extracted with EtOAc (50 mL×3). The organic layer was dried, concentrated and purified by CombiFlash (PE: EtOAc=80%) to give the desired product (220 mg, 37%). MS: M/e 200 (M+1)⁺.

### Step B: 7-formyl-1′,2′-dihydrospiro[cyclopropane-1,3′-pyrrolo[3,2-b]pyridine]-5′-carboxylic acid

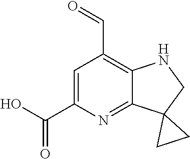 [SMILES: O=Cc1cc(C(=O)O)nc2c1NCC21CC1]

To a solution of 7′-formyl-1′,2′-dihydrospiro[cyclopropane-1,3′-pyrrolo[3,2b]pyridine]-5′-carbonitrile (220 mg, 1.1 mmol) in H₂O (1 mL) was added conc. H₂SO₄ (1 mL) dropwise at 0° C. The reaction mixture was stirred at 100° C. overnight. The reaction was cooled to RT and diluted with H₂O (3 mL), purified by combiflash (C18, ACN: H₂O=30%) to give the desired product (60 mg, 25%). MS: M/e 219 (M+1)⁺.

### Step C: N-(3-((1s, 3s)-3-(cyanomethyl)-1-(4-methyl-4H-1,2,4-triazol-3-yl)cyclo butyl)phenyl)-7′-formyl-1′,2′-dihydrospiro[cyclopropane-1,3′-pyrrolo[3,2-b]pyridine]-5′-carboxamide

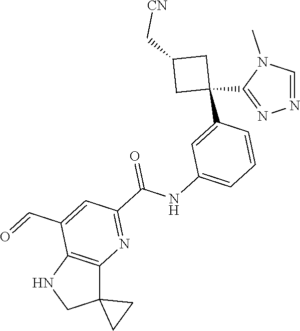 [SMILES: Cn1cnnc1[C@]1(c2cccc(NC(=O)c3cc(C=O)c4c(n3)C3(CC3)CN4)c2)C[C@H](CC#N)C1]

To a solution of 7′-formyl-1′,2′-dihydrospiro[cyclopropane-1,3′-pyrrolo[3,2b]pyridine]-5′-carboxylic acid (60 mg, 0.27 mmol), 2-((1s, 3s)-3-(3-aminophenyl)-3-(4-methyl-4H-1,2,4-triazol-3-yl)cyclobutyl)acetonitrile (110 mg, 0.4 mmol) and DIPEA (70 mg, 0.54 mmol) was added HATU (150 mg, 0.4 mmol) at rt The reaction mixture was stirred at RT for 2 hours. The reaction was quenched by H₂O (10 mL) and extracted with DCM (20 mL×2). The organic phases were concentrated and purified by CombiFlash (C18, ACN: H₂O=40%) to give the desired product (70 mg, 55%). MS: M/e 468 (M+1)⁺.

### Step D: N-(3-((1s, 3s)-3-(cyanomethyl)-1-(4-methyl-4H-1,2,4-triazol-3-yl)cyclo butyl)phenyl)-7′-(((1-methylcyclobutyl)amino)methyl)-1′,2′-dihydrospiro[cyclo propane-1,3′-pyrrolo[3,2-b]pyridine]-5′-carboxamide

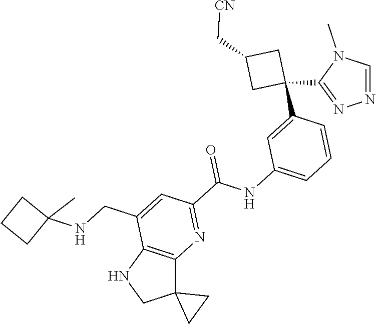 [SMILES: Cn1cnnc1[C@]1(c2cccc(NC(=O)c3cc(CNC4(C)CCC4)c4c(n3)C3(CC3)CN4)c2)C[C@H](CC#N)C1]

A mixture of N-(3-((1s, 3s)-3-(cyanomethyl)-1-(4-methyl-4H-1,2,4-triazol-3-yl)cyclobutyl)phenyl)-7′-formyl-1′,2′-dihydrospiro[cyclopropane-1,3′-pyrrolo[3,2b]pyridine]-5′-carboxamide (15 mg, 0.03 mmol), TEA (6 mg, 0.06 mmol) and 1-methylcyclobutan-1-amine hydrochloride (8 mg, 0.06 mmol) was stirred at RT for 30 min. Then Sodium triacetoxyborohydride (13 mg, 0.06 mmol) was added. The resulting mixture was stirred at RT overnight. The reaction was quenched by H₂O (5 mL) and extracted with DCM (10 mL×2). The organic phases were concentrated and purified by Prep-TLC (DCM:MeOH=85%) to give the desired product (2.98 mg, 18%). ¹H NMR (400 MHz, CD₃OD) δ 8.31 (s, 1H), 8.09 (s, 2H), 7.66 (d, J=7.9 Hz, 1H), 7.41 (t, J=7.8 Hz, 1H), 7.17 (d, J=8.0 Hz, 1H), 4.00 (s, 2H), 3.88 (s, 2H), 3.27-3.25 (m, 1H), 3.05-2.80 (m, 6H), 2.65-2.63 (m, 2H), 2.43-2.40 (m, 2H), 2.20-1.96 (m, 6H), 1.63 (s, 3H), 1.46-1.43 (m, 2H), 1.14-1.09 (m, 2H), 0.94-0.90 (m, 2H) ppm. MS: M/e 537 (M+1)⁺.

### Compound C32: 3,3-dimethyl-7-{[(2-methylpropyl)amino]methyl}-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide

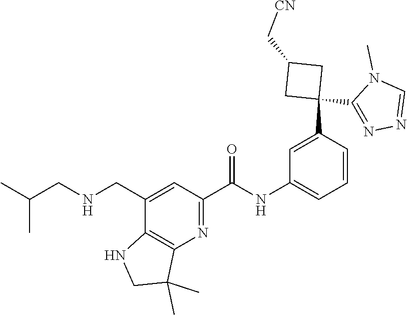 [SMILES: CC(C)CNCc1cc(C(=O)Nc2cccc([C@]3(c4nncn4C)C[C@@H](CC#N)C3)c2)nc2c1NCC2(C)C]

The compound C32 was synthesized starting from the corresponding starting materials according the similar procedures described as those of Compound C22 to give the product (28.1 mg, 38.74%). MS: M/e 527 (M+1)⁺. ¹H NMR (300 MHz, DMSO-d₆) δ 10.03 (s, 1H), 8.30 (s, 1H), 7.89-7.69 (m, 3H), 7.39-7.28 (m, 1H), 7.00 (d, J=7.7 Hz, 1H), 6.36 (s, 1H), 3.59 (s, 2H), 3.40 (s, 2H), 3.19 (s, 3H), 2.87 (s, 2H), 2.77-2.66 (m, 5H), 2.28 (d, J=6.8, 1.8 Hz, 2H), 1.75-1.60 (m, 1H), 1.33 (d, J=1.7 Hz, 6H), 0.88 (s, 3H), 0.85 (s, 3H).

### Compound C33:7-(((cyclopentylmethyl)amino)methyl)-3,3-dimethyl-N-(3-((1s, 3s)-3-methyl-1-(4-methyl-4H-1,2,4-triazol-3-yl)cyclobutyl)phenyl)-2,3-dihydrofuro[3,2-b]pyridine-5-carboxamide

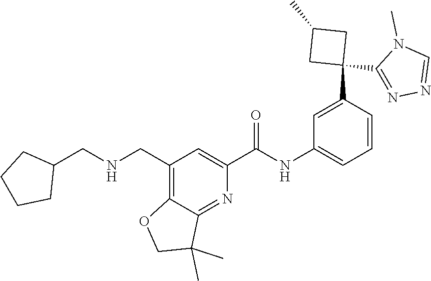 [SMILES: Cn1cnnc1[C@]1(c2cccc(NC(=O)c3cc(CNCC4CCCC4)c4c(n3)C(C)(C)CO4)c2)C[C@H](C)C1]

### Step A: 7-formyl-3,3-dimethyl-2,3-dihydrofuro[3,2-b]pyridine-5-carboxylic acid

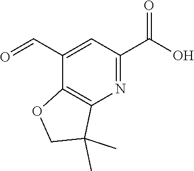 [SMILES: CC1(C)COc2c(C=O)cc(C(=O)O)nc21]

To a solution of methyl 7-formyl-3,3-dimethyl-2,3-dihydrofuro[3,2-b]pyridine-5-carboxylate (200 mg, 0.851 mmol) in HCl (2 mL, 6M) was stirred for 12 hrs at 70° C. After completed, the solvent was concentrated under vacuum. The residue was diluted with water and neutralized with 2 N NaOH and free-dried to obtain the title compound (200 mg, crude). MS: M/e 222 (M+1)⁺

### Step B: 7-formyl-3,3-dimethyl-N-(3-((1s, 3s)-3-methyl-1-(4-methyl-4H-1,2,4-triazol-3-yl)cyclobutyl)phenyl)-2,3-dihydrofuro[3,2-b]pyridine-5-carboxamide

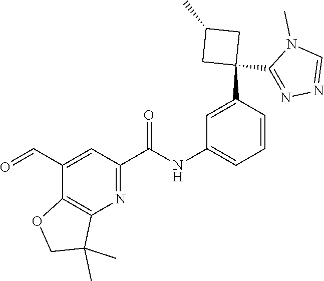 [SMILES: Cn1cnnc1[C@]1(c2cccc(NC(=O)c3cc(C=O)c4c(n3)C(C)(C)CO4)c2)C[C@H](C)C1]

To a solution of 7-formyl-3,3-dimethyl-2,3-dihydrofuro[3,2-b]pyridine-5-carboxylic acid (180 mg, 0.814 mmol), 3-((1s, 3s)-3-methyl-1-(4-methyl-4H-1,2,4-triazol-3-yl)cyclobutyl)aniline (197 mg, 0.814 mmol), DIPEA (315 mg, 2.442 mmol) in DMF (30 mL) was added HATU (464 mg, 1.221 mmol). The reaction mixture was stirred for 6 hrs at 25° C. After completed, the reaction was quenched with aq NH₄Cl (10 mL) and extracted with EA (3×45 mL), dried over Na₂SO₄ and concentrated under vacuum. The residue was purified by combiflash (DCM:MeOH=10:1) to give the title compound (280 mg, 77%). MS: M/e 446 (M+1)⁺

### Step C: 7-(((cyclopentylmethyl)amino)methyl)-3,3-dimethyl-N-(3-((1s, 3s)-3-methyl-1-(4-methyl-4H-1,2,4-triazol-3-yl)cyclobutyl)phenyl)-2,3-dihydrofuro[3,2-b]pyridine-5-carboxamide

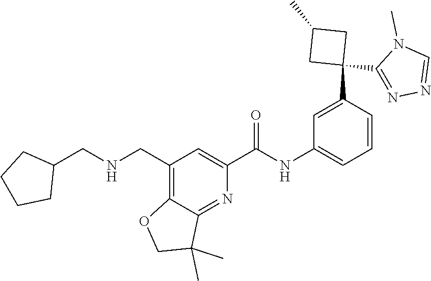 [SMILES: Cn1cnnc1[C@]1(c2cccc(NC(=O)c3cc(CNCC4CCCC4)c4c(n3)C(C)(C)CO4)c2)C[C@H](C)C1]

To a solution of 7-formyl-3,3-dimethyl-N-(3-((1s, 3s)-3-methyl-1-(4-methyl-4H-1,2,4-triazol-3-yl)cyclobutyl)phenyl)-2,3-dihydrofuro[3,2-b]pyridine-5-carboxamide (80 mg, 0.180 mmol), cyclopentylmethanamine hydrochloride (24 mg, 0.360 mmol) and Et₃N (36 mg. 0.360 mmol) in DCM (5 ml) was stirred at room temperature for 1 hour, then added NaBH(OAc)₃ (76 mg, 0.360 mmol). The mixture solution was stirred at 25° C. for 12 hours. The reaction mixture was concentrated under reduced pressure. The crude product was purified by Prep-TLC (DCM:MeOH=15:1) to give the title compound (50 mg, 53%). MS: M/e 529 (M+1)⁺. ¹H NMR (400 MHz, CD₃OD) δ 8.29 (s, 1H), 8.04 (s, 1H), 7.87 (s, 1H), 7.71 (d, J=8.1 Hz, 1H), 7.40 (t, J=7.9 Hz, 1H), 7.13 (d, J=7.8 Hz, 1H), 4.54 (s, 2H), 3.92 (s, 2H), 3.31 (s, 3H), 3.01-2.89 (m, 2H), 2.75-2.52 (m, 5H), 2.10 (dt, J=15.4, 7.7 Hz, 1H), 1.83 (d, J=6.5 Hz, 2H), 1.68-1.53 (m, 4H), 1.46 (s, 6H), 1.26-1.17 (m, 2H), 1.15 (d, J=6.4 Hz, 3H) ppm.

### Compound C34: 7-((((S)-1-cyclopentylethyl)amino)methyl)-3,3-dimethyl-N-(3-((1s,3R)-3-methyl-1-(4-methyl-4H-1,2,4-triazol-3-yl)cyclobutyl)phenyl)-2,3-dihydrofuro[3,2-b]pyridine-5-carboxamide

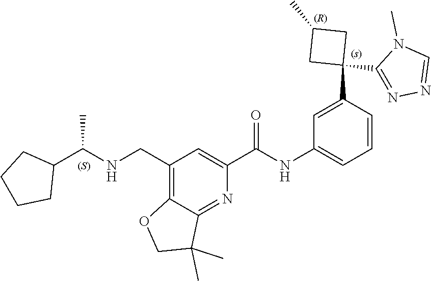

The compound C34 was synthesized starting from the corresponding starting materials according the similar procedures described as those of Compound C33 to give the title compound (43 mg, 45%). MS: M/e 543 (M+1)⁺. ¹H NMR (400 MHz, CD₃OD) δ 8.30 (s, 1H), 8.07 (s, 1H), 7.85 (s, 1H), 7.71 (d, J=8.2 Hz, 1H), 7.40 (t, J=7.9 Hz, 1H), 7.15 (d, J=7.8 Hz, 1H), 4.58 (s, 2H), 4.11 (dd, J=31.3, 14.1 Hz, 2H), 3.30 (s, 3H), 3.00-2.90 (m, 2H), 2.84 (d, J=6.8 Hz, 1H), 2.65 (s, 1H), 2.58 (t, J=10.1 Hz, 2H), 2.03 (d, J=8.2 Hz, 1H), 1.97-1.87 (m, 1H), 1.79 (d, J=4.4 Hz, 1H), 1.73-1.57 (m, 4H), 1.48 (d, J=2.4 Hz, 6H), 1.25 (d, J=6.4 Hz, 5H), 1.15 (d, J=6.3 Hz, 3H). ppm.

### Compound C35:7-((((S)-1-cyclopropylethyl)amino)methyl)-3,3-dimethyl-N-(3-((1s,3R)-3-methyl-1-(4-methyl-4H-1,2,4-triazol-3-yl)cyclobutyl)phenyl)-2,3-dihydrofuro[3,2-b]pyridine-5-carboxamide

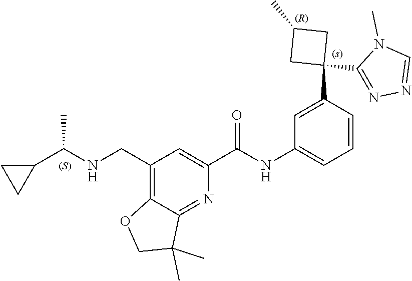

The compound C35 was synthesized starting from the corresponding starting materials according the similar procedures described as those of Compound C33 to give the title compound (30 mg, 47%). MS: M/e 515 (M+1)⁺. ¹H NMR (400 MHz, CD₃OD) δ 8.30 (s, 1H), 8.06 (s, 1H), 7.85 (s, 1H), 7.71 (d, J=8.1 Hz, 1H), 7.40 (t, J=7.9 Hz, 1H), 7.15 (d, J=7.7 Hz, 1H), 4.56 (s, 2H), 4.10 (q, J=14.3 Hz, 2H), 3.31 (s, 3H), 3.00-2.88 (m, 2H), 2.68 (dd, J=15.6, 7.4 Hz, 1H), 2.58 (t, J=10.2 Hz, 2H), 2.24-2.12 (m, 1H), 1.47 (s, 6H), 1.31 (d, J=6.4 Hz, 3H), 1.15 (d, J=6.4 Hz, 3H), 0.86 (ddt, J=13.5, 9.3, 4.6 Hz, 1H), 0.71-0.62 (m, 1H), 0.56 (td, J=8.8, 4.8 Hz, 1H), 0.36 (dq, J=9.6, 4.9 Hz, 1H), 0.18 (td, J=9.7, 4.9 Hz, 1H) ppm.

### Compound C36: N-(3-((1s,3R)-3-(cyanomethyl)-1-(4-methyl-4H-1,2,4-triazol-3-yl)cyclobutyl)phenyl)-7′-((((S)-1-cyclopropylethyl)amino)methyl)-1′,2′-dihydrospiro[cyclopropane-1,3′-pyrrolo[3,2-b]pyridine]-5′-carboxamide

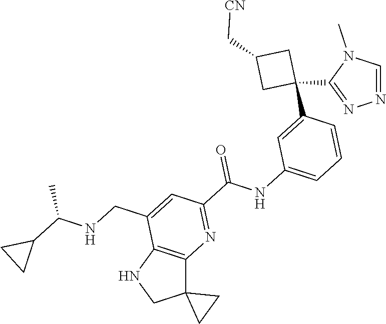 [SMILES: C[C@H](NCc1cc(C(=O)Nc2cccc([C@]3(c4nncn4C)C[C@@H](CC#N)C3)c2)nc2c1NCC21CC1)C1CC1]

A mixture of N-(3-((1s, 3s)-3-(cyanomethyl)-1-(4-methyl-4H-1,2,4-triazol-3-yl)cyclobutyl)phenyl)-7′-formyl-1′,2′-dihydrospiro[cyclopropane-1,3′-pyrrolo[3,2b]pyridine]-5′-carboxamide (15 mg, 0.03 mmol), TEA (6 mg, 0.06 mmol) and (S)-1-cyclopropylethan-1-amine hydrochloride (8 mg, 0.06 mmol) was stirred at RT for 30 min. Then Sodium triacetoxyborohydride (13 mg, 0.06 mmol) was added. The resulting mixture was stirred at RT overnight. The reaction was quenched by H₂O (5 mL) and extracted with DCM (10 mL×2). The organic phases were concentrated and purified by Prep-TLC (DCM:MeOH=85%) to give the desired product (3.45 mg, 20%). ¹H NMR (400 MHz, CD₃OD) δ 8.31 (s, 1H), 7.82 (s, 1H), 7.80 (s, 1H), 7.67 (d, J=8.3 Hz, 1H), 7.41 (t, J=7.9 Hz, 1H), 7.17 (d, J=8.0 Hz, 1H), 4.28-4.13 (m, 2H), 3.89 (s, 2H), 3.03-3.00 (m, 2H), 2.85-2.73 (m, 3H), 2.70-2.67 (m, 1H), 2.65 (d, J=6.0 Hz, 2H), 1.48-1.44 (m, 5H), 1.37 (s, 3H), 1.14-1.10 (m, 2H), 1.07-1.01 (m, 1H), 0.92-0.70 (m, 4H), 0.64-0.56 (m, 1H), 0.41-0.33 (m, 1H) ppm. MS: M/e 537 (M+1)⁺.

### Compound C37: N-(3-((1s, 3s)-3-(cyanomethyl)-1-(4-methyl-4H-1,2,4-triazol-3-yl)cyclobutyl)phenyl)-7,7-dimethyl-4-((((1-methylcyclobutyl)methyl)amino)methyl)-6,7-dihydro-5H-cyclopenta[b]pyridine-2-carboxamide

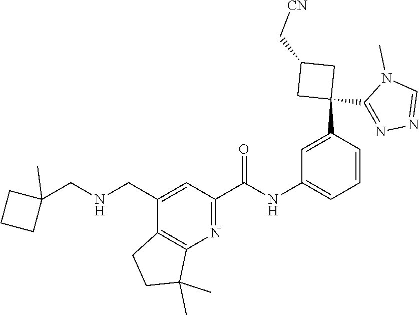 [SMILES: Cn1cnnc1[C@]1(c2cccc(NC(=O)c3cc(CNCC4(C)CCC4)c4c(n3)C(C)(C)CC4)c2)C[C@H](CC#N)C1]

The compound C37 was synthesized starting from the corresponding starting materials according the similar procedures described as those of Compound C16 to give the product (28.8 mg, 34.94% yield). MS: M/e 552 (M+1)⁺; ¹H NMR (400 MHz, DMSO-d₆) δ=10.35-10.30 (m, 1H), 8.35-8.26 (m, 1H), 8.03 (s, 1H), 7.86 (s, 1H), 7.81-7.75 (m, 1H), 7.38 (t, J=8.0 Hz, 1H), 7.08 (d, J=8.2 Hz, 1H), 3.77 (s, 2H), 3.21 (s, 3H), 2.92-2.87 (m, 4H), 2.78-2.66 (m, 6H), 2.45 (s, 2H), 1.99 (t, J=7.2 Hz, 2H), 1.88-1.81 (m, 3H), 1.78-1.71 (m, 1H), 1.64-1.54 (m, 2H), 1.32 (s, 6H), 1.12 (s, 3H)

### Compound C38: N-(3-((1s,3R)-3-(cyanomethyl)-1-(4-methyl-4H-1,2,4-triazol-3-yl)cyclobutyl)phenyl)-4-((((S)-1-cyclobutylethyl)amino)methyl)-7,7-dimethyl-6,7-dihydro-5H-cyclopenta[b]pyridine-2-carboxamide

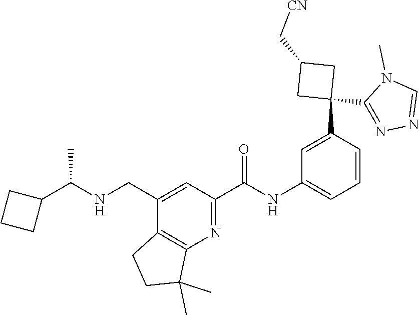 [SMILES: C[C@H](NCc1cc(C(=O)Nc2cccc([C@]3(c4nncn4C)C[C@@H](CC#N)C3)c2)nc2c1CCC2(C)C)C1CCC1]

The compound C38 was synthesized starting from the corresponding starting materials according the similar procedures described as those of Compound C16 to give product (27.39 mg, yield: 47.8%). MS: M/e 552 (M+1)⁺, ¹H NMR (400 MHz, DMSO-d₆) δ=10.32 (s, 1H), 8.31 (s, 1H), 8.02 (s, 1H), 7.86 (s, 1H), 7.82-7.76 (m, 1H), 7.39 (t, J=7.8 Hz, 1H), 7.08 (d, J=8.1 Hz, 1H), 3.85-3.64 (m, 2H), 3.21 (s, 3H), 2.94-2.84 (m, 4H), 2.80-2.72 (m, 3H), 2.72-2.68 (m, 2 H), 2.25-2.15 (m, 1H), 1.99 (t, J=7.2 Hz, 3H), 1.89 (d, J=7.0 Hz, 2H), 1.81-1.62 (m, 4H), 1.33 (d, J=2.4 Hz, 6H), 0.91 (d, J=6.1 Hz, 3H).

### Compound C39: N-(3-((1s,3R)-3-(cyanomethyl)-1-(4-methyl-4H-1,2,4-triazol-3-yl)cyclobutyl)phenyl)-4-((((S)-1-cyclopropylethyl)amino)methyl)-7,7-dimethyl-6,7-dihydro-5H-cyclopenta[b]pyridine-2-carboxamide

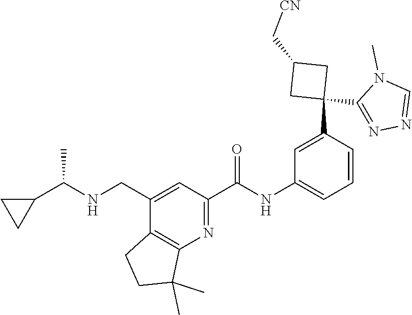 [SMILES: C[C@H](NCc1cc(C(=O)Nc2cccc([C@]3(c4nncn4C)C[C@@H](CC#N)C3)c2)nc2c1CCC2(C)C)C1CC1]

The compound C39 was synthesized starting from the corresponding starting materials according the similar procedures described as those of Compound C16 to give product (30.21 mg, yield: 37.5%). MS: M/e 538 (M+1)⁺; ¹H NMR (400 MHz, DMSO-d₆) δ=10.32 (s, 1H), 8.31 (s, 1H), 8.04 (s, 1H), 7.87 (s, 1H), 7.79 (br d, J=8.0 Hz, 1H), 7.39 (t, J=7.8 Hz, 1H), 7.08 (d, J=8.2 Hz, 1H), 3.82 (br t, J=6.6 Hz, 2H), 3.21 (s, 3H), 2.94-2.84 (m, 4H), 2.75 (br d, J=7.0 Hz, 3H), 2.72-2.66 (m, 2H), 2.29-2.18 (m, 1H), 2.00 (t, J=7.2 Hz, 2H), 1.90 (br t, J=6.2 Hz, 1H), 1.33 (s, 6H), 1.10 (d, J=6.4 Hz, 3H), 0.73-0.61 (m, 1H), 0.48-0.39 (m, 1H), 0.34 (br dd, J=4.1, 8.4 Hz, 1H), 0.22-0.14 (m, 1H), 0.03 (td, J=4.6, 9.2 Hz, 1H) ppm.

### Compound C40: N-(3-((1s, 3s)-3-(cyanomethyl)-1-(4-methyl-4H-1,2,4-triazol-3-yl)cyclobutyl)phenyl)-7,7-dimethyl-4-(((1-methylcyclobutyl)amino)methyl)-6,7-dihydro-5H-cyclopenta[b]pyridine-2-carboxamide

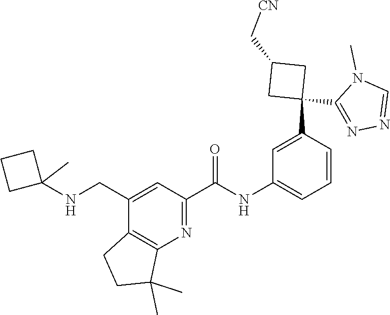 [SMILES: Cn1cnnc1[C@]1(c2cccc(NC(=O)c3cc(CNC4(C)CCC4)c4c(n3)C(C)(C)CC4)c2)C[C@H](CC#N)C1]

The compound C40 was synthesized starting from the corresponding starting materials according the similar procedures described as those of Compound C16 to give the product (27.62 mg, 30.09% yield). MS: M/e 538 (M+1)⁺; ¹H NMR (400 MHz, DMSO-d₆) δ=10.32 (s, 1H), 8.31 (s, 1H), 8.05 (s, 1H), 7.86 (s, 1H), 7.82-7.75 (m, 1H), 7.39 (t, J=8.0 Hz, 1H), 7.08 (d, J=8.0 Hz, 1H), 3.66 (s, 2H), 3.21 (s, 3H), 2.97-2.84 (m, 4H), 2.81-2.65 (m, 5H), 2.08-1.94 (m, 4H), 1.81-1.61 (m, 4H), 1.33 (s, 6H), 1.25 (s, 3H).

### Compound C41: 4-({[(1S)-1-cyclobutylethyl]amino}methyl)-7,7-difluoro-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide

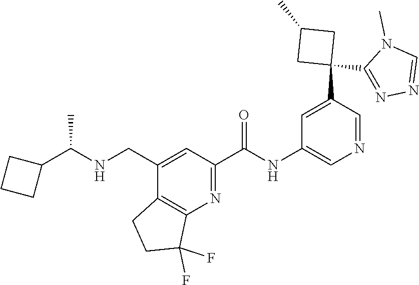 [SMILES: C[C@H](NCc1cc(C(=O)Nc2cncc([C@]3(c4nncn4C)C[C@@H](C)C3)c2)nc2c1CCC2(F)F)C1CCC1]

### Step 1: methyl (S)-4-(((1-cyclobutylethyl)amino)methyl)-7,7-difluoro-6,7-dihydro-5H-cyclopenta[b]pyridine-2-carboxylate

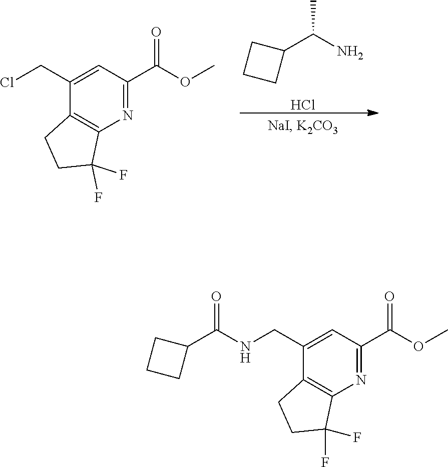

A solution of methyl 4-(chloromethyl)-7,7-difluoro-5H,6H-cyclopenta[b]pyridine-2-carboxylate (35 mg, 0.079 mmol, 1 equiv, 59.2%), NaI (12 mg, 0.076 mmol, 0.96 equiv, 95%) and K₂CO₃ (23 mg, 0.158 mmol, 2.00 equiv, 95%) in MeCN (2 mL, 95%) was stirred for 2 hrs at 70° C. under nitrogen atmosphere. The mixture was allowed to cool down to room temperature. The resulting mixture was concentrated under reduced pressure. The residue was purified by silica gel column chromatography, eluted with CH₂Cl₂/MeOH (10:1) to afford methyl 4-({[(1S)-1-cyclobutylethyl]amino}methyl)-7,7-difluoro-5H,6H-cyclopenta[b]pyridine-2-carboxylate (25 mg, 44.38%). MS: M/e 325 (M+1)⁺.

### Step 2: 4-({[(1S)-1-cyclobutylethyl]amino}methyl)-7,7-difluoro-5H,6H-cyclopenta[b]pyridine-2-carboxylic acid

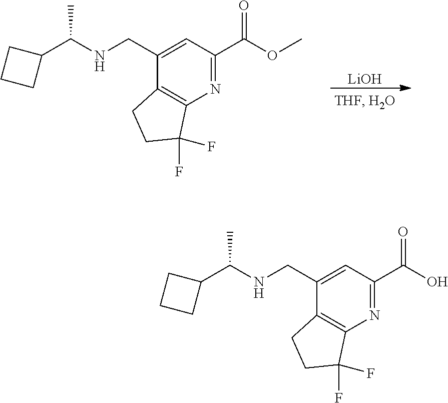 [SMILES: COC(=O)c1cc(CN[C@@H](C)C2CCC2)c2c(n1)C(F)(F)CC2 ; "LiOH" ; C[C@H](NCc1cc(C(=O)O)nc2c1CCC2(F)F)C1CCC1]

To a stirred solution of methyl 4-({[(1S)-1-cyclobutylethyl]amino}methyl)-7,7-difluoro-5H,6H-cyclopenta[b]pyridine-2-carboxylate (25 mg, 0.035 mmol, 1 equiv, 45.6%) in THF (0.6 mL, 95%) and H₂O (0.2 mL, 10.547 mmol, 300.09 equiv, 95%) was added LiOH (2 mg, 0.079 mmol, 2.26 equiv, 95%) in portions at 0° C. The resulting mixture was stirred for 1 h at room temperature. The mixture was neutralized to pH=7 with HCl (aq.). The resulting mixture was concentrated under reduced pressure. The residue was purified by reversed-phase flash chromatography with the following conditions: column, C18 silica gel; mobile phase, MeCN in Water (0.1% TFA), 20% to 25% gradient in 2 min; This resulted in 4-({[(1S)-1-cyclobutylethyl]amino}methyl)-7,7-difluoro-5H,6H-cyclopenta[b]pyridine-2-carboxylic acid (9 mg, 72.53%). MS: M/e 311 (M+1)⁺.

### Step 3: 4-({[(1S)-1-cyclobutylethyl]amino}methyl)-7,7-difluoro-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide

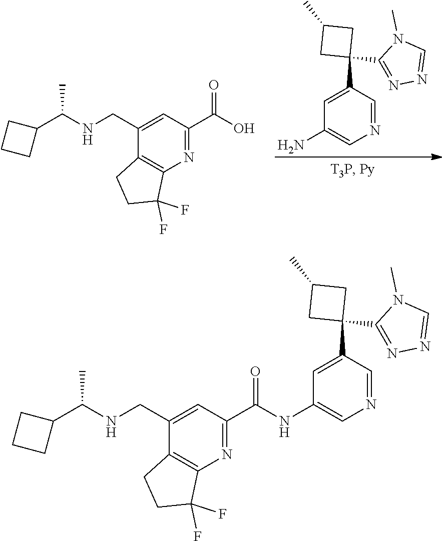 [SMILES: C[C@H](NCc1cc(C(=O)O)nc2c1CCC2(F)F)C1CCC1 ; Cn1cnnc1[C@]1(c2cncc(N)c2)C[C@H](C)C1 ; C[C@H](NCc1cc(C(=O)Nc2cncc([C@]3(c4nncn4C)C[C@@H](C)C3)c2)nc2c1CCC2(F)F)C1CCC1]

To a stirred solution of 4-({[(1S)-1-cyclobutylethyl]amino}methyl)-7,7-difluoro-5H,6H-cyclopenta[b]pyridine-2-carboxylic acid (7 mg, 0.020 mmol, 1 equiv, 87.9%) and 5-[(1r, 3s)-3-methyl-1-(4-methyl-1,2,4-triazol-3-yl)cyclobutyl]pyridin-3-amine (4 mg, 0.016 mmol, 0.79 equiv, 95%) in Pyridine (40 mg, 0.480 mmol, 24.23 equiv, 95%) was added T₃P in EtOAc (126 mg, 0.198 mmol, 9.99 equiv, 50%) dropwise at room temperature. The resulting mixture was stirred for 30 min at room temperature under nitrogen atmosphere. The resulting mixture was concentrated under reduced pressure. The crude product was purified by Prep-HPLC with the following conditions (2 #SHIMADZU (HPLC-01)): Column, XBridge Prep OBD C18 Column, 19*250 mm, 5 μm; mobile phase, 10 mmol NH4HCO₃+0.05% NH₃H2O and ACN (19% ACN up to 49% in 8 min); This resulted in 4-({[(1S)-1-cyclobutylethyl]amino}methyl)-7,7-difluoro-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide (1.3 mg, 11.91%). MS: M/e 536 (M+1)⁺. ¹H NMR (300 MHz, DMSO-d₆) δ 10.68 (s, 1H), 9.08 (d, J=2.2 Hz, 1H), 8.39-8.20 (m, 4H), 3.94-3.75 (m, 2H), 3.22 (s, 3H), 3.10 (s, 2H), 2.96-2.83 (m, 2H), 2.81-2.63 (m, 2H), 2.63-2.52 (m, 3H), 2.33-1.62 (m, 9H), 1.10 (d, J=5.3 Hz, 3H), 0.92 (d, J=6.2 Hz, 3H) ppm.

### Compound C42: N-(3-((1s, 3s)-3-(cyanomethyl)-1-(4-methyl-4H-1,2,4-triazol-3-yl)cyclobutyl)phenyl)-7,7-difluoro-4-(((1-methylcyclobutyl)amino)methyl)-6,7-dihydro-5H-cyclopenta[b]pyridine-2-carboxamide

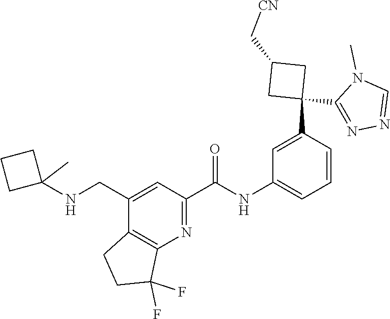 [SMILES: Cn1cnnc1[C@]1(c2cccc(NC(=O)c3cc(CNC4(C)CCC4)c4c(n3)C(F)(F)CC4)c2)C[C@H](CC#N)C1]

A mixture of (2-((3-((1s, 3s)-3-(cyanomethyl)-1-(4-methyl-4H-1,2,4-triazol-3-yl)cyclobutyl)phenyl)carbamoyl)-7,7-difluoro-6,7-dihydro-5H-cyclopenta[b]pyridin-4-yl)methyl methanesulfonate (0.12 g, 241.48 umol, 1 eq), 1-methylcyclobutanamine (146.83 mg, 1.21 mmol, 5 eq, HCl), Cs₂CO₃ (157.36 mg, 482.96 umol, 2 eq), TEA (122.18 mg, 1.21 mmol, 168.06 uL, 5 eq), DMF (4 mL) was stirred at 25° C. for 12 hrs. The mixture was poured into 40 ml H₂O, extracted with Dichloromethane (40 mL*2), washed with brine(20 mL*2), the combined organic layers were dried over Na₂SO₄, filtered and concentrated under reduced pressure to give a residue. The residue was purified by Prep-HPLC(column: Waters Xbridge BEH C18 100*30 mm*10 um; mobile phase: [water (NH₄HCO₃)-ACN]; B %: 35%-65%, 8 min). Compound N-(3-((1s, 3s)-3-(cyanomethyl)-1-(4-methyl-4H-1,2,4-triazol-3-yl)cyclobutyl)phenyl)-7,7-difluoro-4-(((1-methylcyclobutyl)amino)methyl)-6,7-dihydro-5H-cyclopenta[b]pyridine-2-carboxamide (67.16 mg, 50.97% yield) was obtained. MS: M/e 546 (M+1)⁺; ¹H NMR (400 MHz, DMSO-d₆) δ ppm 10.45 (s, 1H), 8.34 (d, J=18.02 Hz, 2H), 7.83-7.97 (m, 2H), 7.38 (t, J=7.94 Hz, 1H), 7.04 (d, J=7.74 Hz, 1H), 3.75 (s, 2H), 3.21 (s, 3H), 3.12 (s, 2H), 2.64-2.92 (m, 10H), 1.94-2.06 (m, 2H), 1.61-1.79 (m, 4H), 1.24 (s, 3H) ppm.

### Compound C43: N-(3-((1s,3R)-3-(cyanomethyl)-1-(4-methyl-4H-1,2,4-triazol-3-yl)cyclobutyl)phenyl)-4-((((S)-1-cyclopropylethyl)amino)methyl)-7,7-difluoro-6,7-dihydro-5H-cyclopenta[b]pyridine-2-carboxamide

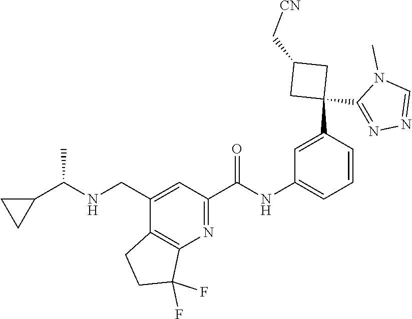 [SMILES: C[C@H](NCc1cc(C(=O)Nc2cccc([C@]3(c4nncn4C)C[C@@H](CC#N)C3)c2)nc2c1CCC2(F)F)C1CC1]

### Step 1: N-(3-((1s, 3s)-3-(cyanomethyl)-1-(4-methyl-4H-1,2,4-triazol-3-yl)cyclobutyl)phenyl)-7,7-difluoro-4-(hydroxymethyl)-6,7-dihydro-5H-cyclopenta[b]pyridine-2-carboxamide

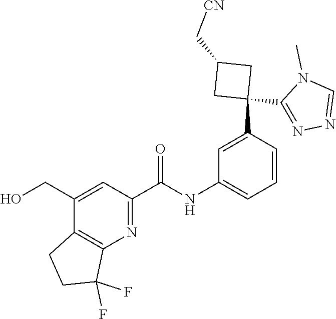 [SMILES: Cn1cnnc1[C@]1(c2cccc(NC(=O)c3cc(CO)c4c(n3)C(F)(F)CC4)c2)C[C@H](CC#N)C1]

A mixture of 2-[3-(3-aminophenyl)-3-(4-methyl-1,2,4-triazol-3-yl)cyclobutyl]acetonitrile (436.62 mg, 1.63 mmol), [7,7-difluoro-4-(hydroxymethyl)-5,6-dihydrocyclopenta[b]pyridine-2-carbonyl]oxylithium (0.48 g, 1.63 mmol, 80% purity), HATU (807.32 mg, 2.12 mmol), DIEA (422.16 mg, 3.27 mmol, 568.95 uL), DMF (37 mL) was stirred at 25° C. for 2 hrs. The mixture was poured into 150 ml H₂O, extracted with Dichloromethane (150 mL*2), washed with brine(150 mL*2), the combined organic layers were dried over Na₂SO₄, filtered and concentrated under reduced pressure to give a residue. The residue was purified by prep-HPLC(column: Phenomenex C18 80*40 mm*3 um; mobile phase: [water (NH₄HCO₃)-ACN]; B %: 25%-55%, 8 min). Compound N-(3-((1s, 3s)-3-(cyanomethyl)-1-(4-methyl-4H-1,2,4-triazol-3-yl)cyclobutyl)phenyl)-7,7-difluoro-4-(hydroxymethyl)-6,7-dihydro-5H-cyclopenta[b]pyridine-2-carboxamide (0.46 g, 961.35 umol, 58.86% yield) was obtained.

### Step 2: (2-((3-((1s, 3s)-3-(cyanomethyl)-1-(4-methyl-4H-1,2,4-triazol-3-yl)cyclobutyl)phenyl)carbamoyl)-7,7-difluoro-6,7-dihydro-5H-cyclopenta[b]pyridin-4-yl)methyl methanesulfonate

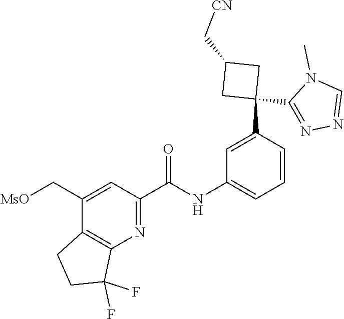 [SMILES: Cn1cnnc1[C@]1(c2cccc(NC(=O)c3cc(COS(C)(=O)=O)c4c(n3)C(F)(F)CC4)c2)C[C@H](CC#N)C1]

In a mixture of N-[3-[3-(cyanomethyl)-1-(4-methyl-1,2,4-triazol-3-yl)cyclobutyl]phenyl]-7,7-difluoro-4-(hydroxymethyl)-5,6-dihydrocyclopenta[b]pyridine-2-carboxamide (0.15 g, 313.48 umol), DMF (9 mL), MsCl (172.37 mg, 1.50 mmol, 116.46 uL) was added TEA (253.77 mg, 2.51 mmol, 349.07 uL) at 0° C., then the mixture was warmed to 20° C. for 1 h. The mixture was poured into 40 ml H₂O, extracted with Dichloromethane (30 mL*2), washed with brine(20 mL*2), the combined organic layers were dried over Na₂SO₄, filtered and concentrated under reduced pressure to give a residue. Compound (2-((3-((1s, 3s)-3-(cyanomethyl)-1-(4-methyl-4H-1,2,4-triazol-3-yl)cyclobutyl)phenyl)carbamoyl)-7,7-difluoro-6,7-dihydro-5H-cyclopenta[b]pyridin-4-yl)methyl methanesulfonate (0.15 g, 301.85 umol, 96.29% yield) was obtained. MS: M/e 557 (M+1)⁺

### Step 3: N-(3-((1s,3R)-3-(cyanomethyl)-1-(4-methyl-4H-1,2,4-triazol-3-yl)cyclobutyl)phenyl)-4-((((S)-1-cyclopropylethyl)amino)methyl)-7,7-difluoro-6,7-dihydro-5H-cyclopenta[b]pyridine-2-carboxamide

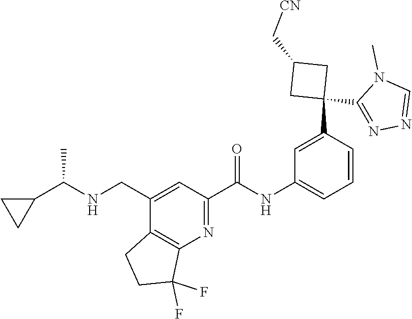 [SMILES: C[C@H](NCc1cc(C(=O)Nc2cccc([C@]3(c4nncn4C)C[C@@H](CC#N)C3)c2)nc2c1CCC2(F)F)C1CC1]

A mixture of 4-(chloromethyl)-N-[3-[3-(cyanomethyl)-1-(4-methyl-1,2,4-triazol-3-yl)cyclobutyl]phenyl]-7,7-difluoro-5,6-dihydrocyclopenta[b]pyridine-2-carboxamide (0.052 g, 104.64 umol), (1S)-1-cyclopropylethanamine (127.25 mg, 1.05 mmol, HCl), Cs₂CO₃ (68.19 mg, 209.28 umol), TEA (105.89 mg, 1.05 mmol, 145.65 uL), DMF (5 mL) was stirred at 30° C. for 12 hrs. The mixture was poured into 40 ml H₂O, extracted with Dichloromethane (30 mL*2), washed with brine (20 mL*2), the combined organic layers were dried over Na₂SO₄, filtered and concentrated under reduced pressure to give a residue. The residue was purified by prep-HPLC(column: Phenomenex C18 80*40 mm*3 um; mobile phase: [water (NH₄HCO₃)-ACN]; B %: 30%-50%, 8 min). Compound N-(3-((1s,3R)-3-(cyanomethyl)-1-(4-methyl-4H-1,2,4-triazol-3-yl)cyclobutyl)phenyl)-4-((((S)-1-cyclopropylethyl)amino)methyl)-7,7-difluoro-6,7-dihydro-5H-cyclopenta[b]pyridine-2-carboxamide (21.12 mg, 38.71 umol, 36.99% yield) was obtained. MS: M/e 546 (M+1)⁺; ¹H NMR (400 MHz, DMSO-d₆) δ ppm 10.44 (s, 1H), 8.36 (s, 1H), 8.31 (s, 1H), 7.87-7.96 (m, 2H), 7.37 (t, J=7.94 Hz, 1H), 7.04 (d, J=8.25 Hz, 1H), 3.91 (d, J=7.50 Hz, 2H), 3.21 (s, 3H), 3.10 (s, 2H), 2.82-2.90 (m, 2H), 2.65-2.80 (m, 7H), 2.40-2.50 (m, 1H), 1.88-1.98 (m, 1H), 1.11 (d, J=6.24 Hz, 3H), 0.60-0.74 (m, 1H), 0.28-0.47 (m, 2H), 0.13-0.23 (m, 1H), −0.03-0.06 (m, 1H) ppm.

### Compound C44: 3,3-dimethyl-7-{[(1-methylcyclopropyl)amino]methyl}-N-{3-[(1r, 3r)-3-cyano-1-[(4-methyl-1,2,4-triazol-3-yl)methyl]cyclobutyl]phenyl}-1H,2H-pyrrolo[3,2-b]pyridine-5-carboxamide

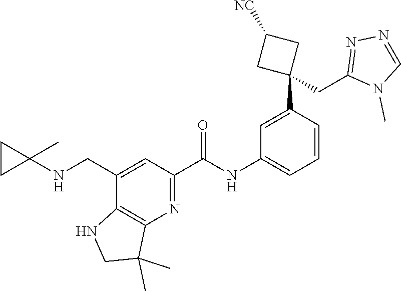 [SMILES: Cn1cnnc1C[C@]1(c2cccc(NC(=O)c3cc(CNC4(C)CC4)c4c(n3)C(C)(C)CN4)c2)C[C@H](C#N)C1]

### Step 1: 7-formyl-3,3-dimethyl-N-{3-[(1r, 3r)-3-cyano-1-[(4-methyl-1,2,4-triazol-3-yl)methyl]cyclobutyl]phenyl}-1H,2H-pyrrolo[3,2-b]pyridine-5-carboxamide

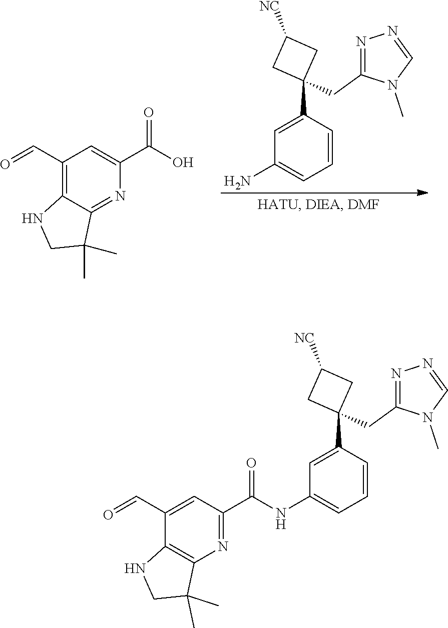 [SMILES: CC1(C)CNc2c(C=O)cc(C(=O)O)nc21 ; Cn1cnnc1C[C@]1(c2cccc(N)c2)C[C@H](C#N)C1 ; Cn1cnnc1C[C@]1(c2cccc(NC(=O)c3cc(C=O)c4c(n3)C(C)(C)CN4)c2)C[C@H](C#N)C1]

A solution of 7-formyl-3,3-dimethyl-1H,2H-pyrrolo[3,2-b]pyridine-5-carboxylic acid (220 mg, 0.700 mmol, 0.99 equiv, 70.1%) in DMF (20 mL) was treated with HATU (424 mg, 1.059 mmol, 1.50 equiv, 95%) at 0° C. followed by the addition of DIEA (0.39 mL, 2.121 mmol, 3 equiv, 95%) dropwise at 0° C. The resulting mixture was stirred for 0.5 h at room temperature. To the mixture was added (1r, 3r)-3-(3-aminophenyl)-3-[(4-methyl-1,2,4-triazol-3-yl)methyl]cyclobutane-1-carbonitrile (200 mg, 0.707 mmol, 1.00 equiv, 94.5%) in portions at 0° C. The resulting mixture was stirred for additional 2 hrs at room temperature. The resulting mixture was diluted with EtOAc (100 mL). The resulting mixture was washed with 3×20 mL of brine. The resulting mixture was concentrated under reduced pressure. The residue was purified by silica gel column chromatography, eluted with CH₂Cl₂/MeOH (12:1) to afford 7-formyl-3,3-dimethyl-N-{3-[(1r, 3r)-3-cyano-1-[(4-methyl-1,2,4-triazol-3-yl)methyl]cyclobutyl]phenyl}-1H,2H-pyrrolo[3,2-b]pyridine-5-carboxamide (310 mg, 91.89%). MS: M/e 470 (M+1)⁺

### Step 2: 3,3-dimethyl-7-{[(1-methylcyclopropyl)amino]methyl}-N-{3-[(1r, 3r)-3-cyano-1-[(4-methyl-1,2,4-triazol-3-yl)methyl]cyclobutyl]phenyl}-1H,2H-pyrrolo[3,2-b]pyridine-5-carboxamide

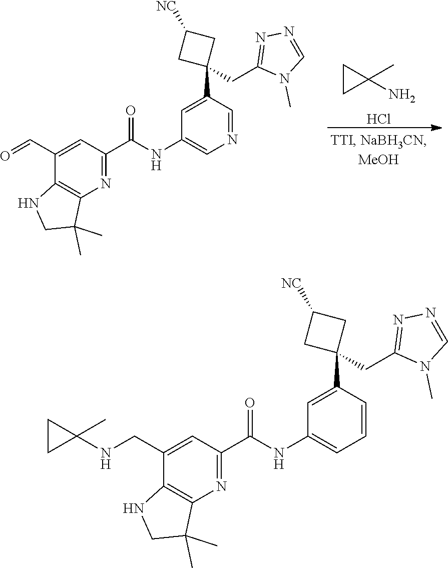

A solution of 7-formyl-3,3-dimethyl-N-{3-[(1r, 3r)-3-cyano-1-[(4-methyl-1,2,4-triazol-3-yl)methyl]cyclobutyl]phenyl}-1H,2H-pyrrolo[3,2-b]pyridine-5-carboxamide (65 mg, 0.136 mmol, 1 equiv, 98.4%), 1-methylcyclopropan-1-amine hydrochloride (39 mg, 0.344 mmol, 2.53 equiv, 95%) and tetrakis(propan-2-yloxy)titanium (164 mg, 0.548 mmol, 4.02 equiv, 95%) in MeOH (4 mL) was stirred for 30 min at room temperature. The resulting mixture was stirred for 1 h at room temperature. The resulting mixture was concentrated under reduced pressure. The residue was purified by silica gel column chromatography, eluted with CH₂Cl₂/MeOH (6:1) to afford crude product. The crude product was purified by Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (30% ACN up to 60% in 8 min. This resulted in 3,3-dimethyl-7-{[(1-methylcyclopropyl)amino]methyl}-N-{3-[(1r, 3r)-3-cyano-1-[(4-methyl-1,2,4-triazol-3-yl)methyl]cyclobutyl]phenyl}-1H,2H-pyrrolo[3,2-b]pyridine-5-carboxamide (27.0 mg, 37.55%). MS: M/e 525 (M+1)⁺. ¹H NMR (300 MHz, DMSO-d₆) δ 9.94 (s, 1H), 8.21-8.14 (m, 1H), 7.81-7.71 (m, 2H), 7.52-7.44 (m, 1H), 7.29-7.16 (m, 1H), 6.67-6.57 (m, 1H), 6.33-6.25 (m, 1H), 3.64 (s, 2H), 3.41 (s, 2H), 3.30-3.14 (m, 3H), 2.89-2.75 (m, 6H), 2.53-2.46 (m, 1H), 2.36 (s, 1H), 1.37-1.29 (m, 6H), 1.27-1.20 (m, 3H), 0.59-0.49 (m, 2H), 0.40-0.25 (m, 2H).

### Compound C45: 7-{[(cyclopentylmethyl)amino]methyl}-3,3-dimethyl-N-{3-[(1r, 3s)-3-methyl-1-(4-methyl-1,2,4-triazol-3-yl)cyclobutyl]phenyl}-1H,2H-pyrrolo[3,2-b]pyridine-5-carboxamide

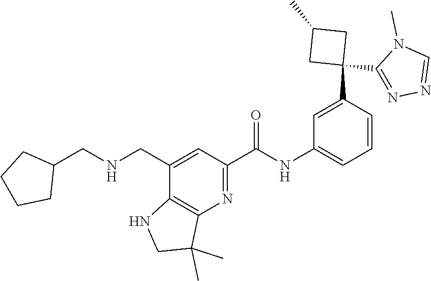 [SMILES: Cn1cnnc1[C@]1(c2cccc(NC(=O)c3cc(CNCC4CCCC4)c4c(n3)C(C)(C)CN4)c2)C[C@H](C)C1]

### Step 1: 7-formyl-3,3-dimethyl-N-{3-[(1r, 3s)-3-methyl-1-(4-methyl-1,2,4-triazol-3-yl)cyclobutyl]phenyl}-1H, 2H-pyrrolo[3,2-b]pyridine-5-carboxamide

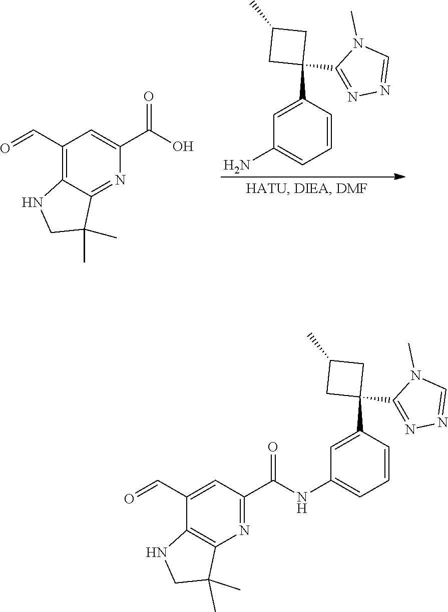 [SMILES: CC1(C)CNc2c(C=O)cc(C(=O)O)nc21 ; Cn1cnnc1[C@]1(c2cccc(N)c2)C[C@H](C)C1 ; Cn1cnnc1[C@]1(c2cccc(NC(=O)c3cc(C=O)c4c(n3)C(C)(C)CN4)c2)C[C@H](C)C1]

A solution of 7-formyl-3,3-dimethyl-1H,2H-pyrrolo[3,2-b]pyridine-5-carboxylic acid (320 mg, 1.019 mmol, 1.01 equiv, 70.1%) in DMF (30 mL) was treated with HATU (608 mg, 1.519 mmol, 1.50 equiv, 95%) at 0° C. followed by the addition of DIEA (0.56 mL, 3.039 mmol, 3 equiv, 95%) dropwise at 0° C. The resulting mixture was stirred for 0.5 h at room temperature. To the mixture was added 3-[(1r, 3s)-3-methyl-1-(4-methyl-1,2,4-triazol-3-yl)cyclobutyl]aniline (300 mg, 1.013 mmol, 1.00 equiv, 81.8%) in portions at 0° C. The resulting mixture was stirred for additional 2 hrs at room temperature. The resulting mixture was diluted with EtOAc (100 mL). The resulting mixture was washed with 3×30 mL of brine. The resulting mixture was concentrated under reduced pressure. The residue was purified by silica gel column chromatography, eluted with CH₂Cl₂/MeOH (12:1) to afford 7-formyl-3,3-dimethyl-N-{3-[(1r, 3s)-3-methyl-1-(4-methyl-1,2,4-triazol-3-yl)cyclobutyl]phenyl}-1H,2H-pyrrolo[3,2-b]pyridine-5-carboxamide (400 mg, 88.76%). MS: M/e 445 (M+1)⁺.

### Step 2: 7-{[(cyclopentylmethyl)amino]methyl}-3,3-dimethyl-N-{3-[(1r, 3s)-3-methyl-1-(4-methyl-1,2,4-triazol-3-yl)cyclobutyl]phenyl}-1H,2H-pyrrolo[3,2-b]pyridine-5-carboxamide

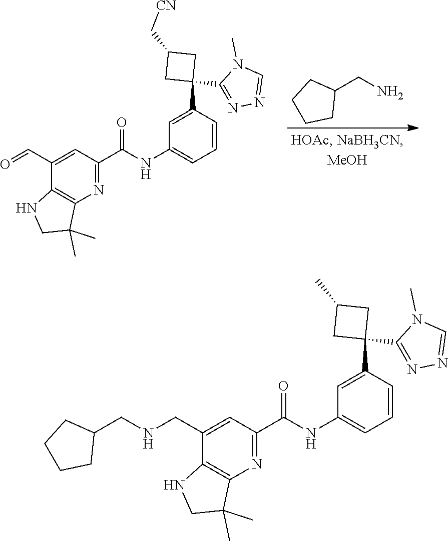

A solution of 7-formyl-3,3-dimethyl-N-{3-[(1r, 3s)-3-methyl-1-(4-methyl-1,2,4-triazol-3-yl)cyclobutyl]phenyl}-1H,2H-pyrrolo[3,2-b]pyridine-5-carboxamide (65 mg, 0.146 mmol, 1 equiv, 99.9%), HOAc (0.04 mL, 0.663 mmol, 4.54 equiv, 95%) and 1-cyclopentylmethanamine (30 mg, 0.287 mmol, 1.97 equiv, 95%) in MeOH (4 mL, 95%) was stirred for 1 h at room temperature. To the above mixture was added NaBH₃CN (13 mg, 0.197 mmol, 1.35 equiv, 95%) in portions at 0° C. The resulting mixture was stirred for additional 1 h at room temperature. The resulting mixture was concentrated under reduced pressure. The crude product was purified by Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃+0.1% NH₃·H₂O) and ACN (40% ACN up to 70% in 8 min); This resulted in 7-{[(cyclopentylmethyl)amino]methyl}-3,3-dimethyl-N-{3-[(1r, 3s)-3-methyl-1-(4-methyl-1,2,4-triazol-3-yl)cyclobutyl]phenyl}-1H,2H-pyrrolo[3,2-b]pyridine-5-carboxamide (46.9 mg, 59.87%). MS: M/e 529 (M+1)⁺. ¹H NMR (300 MHz, DMSO-d₆) δ 10.03 (s, 1H), 8.28 (s, 1H), 7.89-7.68 (m, 3H), 7.33 (t, J=7.9 Hz, 1H), 7.02-6.93 (m, 1H), 6.37 (s, 1H), 3.61 (s, 2H), 3.42 (s, 2H), 3.18 (s, 3H), 2.89-2.78 (m, 2H), 2.59-2.51 (m, 2H), 2.40 (d, J=7.1 Hz, 2H), 2.19 (s, 1H), 2.06-1.88 (m, 1H), 1.79-1.62 (m, 2H), 1.60-1.42 (m, 4H), 1.34 (s, 6H), 1.27-1.03 (m, 6H).

### Compound C46: 7-({[(1S)-1-cyclobutylethyl]amino}methyl)-3,3-dimethyl-N-{3-[(1r, 3s)-3-methyl-1-(4-methyl-1,2,4-triazol-3-yl)cyclobutyl]phenyl}-1H,2H-pyrrolo[3,2-b]pyridine-5-carboxamide

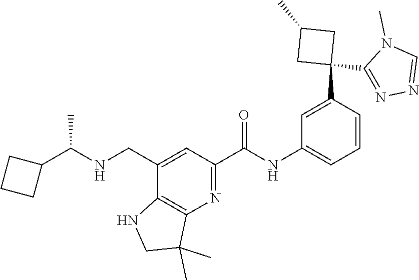 [SMILES: C[C@H](NCc1cc(C(=O)Nc2cccc([C@]3(c4nncn4C)C[C@@H](C)C3)c2)nc2c1NCC2(C)C)C1CCC1]

To a stirred mixture of 7-formyl-3,3-dimethyl-N-{3-[(1r, 3s)-3-methyl-1-(4-methyl-1,2,4-triazol-3-yl)cyclobutyl]phenyl}-1H,2H-pyrrolo[3,2-b]pyridine-5-carboxamide (65 mg, 0.146 mmol, 1 equiv, 99.9%) and (1S)-1-cyclobutylethanamine hydrochloride (42 mg, 0.294 mmol, 2.01 equiv, 95%) in MeOH (3 mL) were added tetrakis(propan-2-yloxy)titanium (175 mg, 0.584 mmol, 4 equiv, 95%) dropwise at room temperature. The resulting mixture was stirred for 1.5 h at room temperature. To the above mixture was added NaBH₃CN (20 mg, 0.302 mmol, 2 equiv, 95%) in portions. The resulting mixture was stirred for additional 2 hrs at room temperature. The resulting mixture was concentrated under vacuum. The residue was purified by silica gel column chromatography, eluted with CH₂Cl₂/MeOH (8:1). The crude product (80 mg) was purified by Prep-HPLC with the following conditions (2 #SHIMADZU (HPLC-01)): Column, XBridge Shield RP18 OBD Column, 30*150 mm, 5 μm; mobile phase, Water (10 mmol/L NH₄HCO₃+0.1% NH₃·H₂O) and ACN (40% ACN up to 70% in 8 min); This resulted in 7-({[(1S)-1-cyclobutylethyl]amino}methyl)-3,3-dimethyl-N-{3-[(1r, 3s)-3-methyl-1-(4-methyl-1,2,4-triazol-3-yl)cyclobutyl]phenyl}-1H,2H-pyrrolo[3,2-b]pyridine-5-carboxamide (44.1 mg, 56.41%). MS: M/e 528 (M+1)⁺. ¹H NMR (300 MHz, DMSO-d₆) δ 10.03 (s, 1H), 8.29 (s, 1H), 7.88-7.67 (m, 3H), 7.36-7.28 (m, 1H), 7.04-6.91 (m, 1H), 6.37 (s, 1H), 3.72-3.53 (m, 2H), 3.42 (d, J=1.4 Hz, 2H), 3.19 (s, 3H), 2.92-2.74 (m, 2H), 2.54 (s, 2H), 2.47-2.35 (m, 1H), 2.25-2.11 (m, 1H), 2.06-1.93 (m, 1H), 1.93-1.82 (m, 2H), 1.80-1.56 (m, 4H), 1.34 (s, 6H), 1.09 (d, J=5.3 Hz, 3H), 0.89 (d, J=6.2 Hz, 3H).

### Compound C47: 7-{[(3R,4S)-4-fluoro-3-methylpiperidin-1-yl]methyl}-3,3-dimethyl-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide

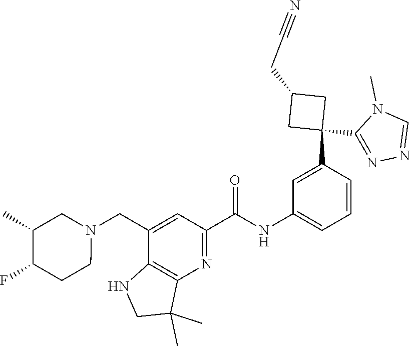 [SMILES: C[C@@H]1CN(Cc2cc(C(=O)Nc3cccc([C@]4(c5nncn5C)C[C@@H](CC#N)C4)c3)nc3c2NCC3(C)C)CC[C@@H]1F]

To a stirred solution of 7-formyl-3,3-dimethyl-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide (60 mg, 0.125 mmol, 1 equiv, 97.6%) and (3R,4S)-4-fluoro-3-methylpiperidine hydrochloride (40.34 mg, 0.250 mmol, 2 equiv, 95%) in MeOH (8 mL, 100%) were added tetrakis(propan-2-yloxy)titanium (149.25 mg, 0.500 mmol, 4 equiv, 95%) at room temperature. The resulting mixture was stirred for 1 h at room temperature under nitrogen atmosphere. To the above mixture was added NaBH₃CN (16.50 mg, 0.250 mmol, 2 equiv, 95%) in portions at room temperature. The resulting mixture was stirred for additional 1 h at room temperature. The resulting mixture was concentrated under reduced pressure. The residue was purified by silica gel column chromatography, eluted with CH₂Cl₂/MeOH (12:1). The crude product was purified by Chiral-Prep-HPLC with the following conditions (2 #SHIMADZU (HPLC-01)): Column, XBridge Prep OBD C18 Column, 30*150 mm, 5 μm; mobile phase, Water (10 mmol/L NH₄HCO₃+0.1% NH₃·H₂O) and ACN (27% ACN up to 57% in 8 min); This resulted in 7-{[(3R,4S)-4-fluoro-3-methylpiperidin-1-yl]methyl}-3,3-dimethyl-N-{3-[(1s, 3s)-3-(cyanomethyl)-1-(4-methyl-1,2,4-triazol-3-yl)cyclobutyl]phenyl}-1H,2H-pyrrolo[3,2-b]pyridine-5-carboxamide (22.1 mg, 29.93%). MS: M/e 571 (M+1)⁺. ¹H NMR (300 MHz, DMSO-d₆) δ 10.04 (s, 1H), 8.31 (s, 1H), 7.89-7.82 (m, 1H), 7.77-7.67 (m, 2H), 7.41-7.29 (m, 1H), 7.07-6.98 (m, 1H), 6.37 (s, 1H), 4.76-4.53 (m, 1H), 3.46-3.39 (m, 4H), 3.35 (s, 1H), 3.21 (s, 3H), 2.88 (s, 2H), 2.79-2.66 (m, 5H), 2.51 (d, J=5.6 Hz, 1H), 2.29-2.15 (m, 1H), 2.07-1.66 (m, 4H), 1.34 (s, 6H), 0.90 (d, J=6.6 Hz, 3H).

### Compound C48: N-(3-((1r, 3r)-3-cyano-1-((4-methyl-4H-1,2,4-triazol-3-yl)methyl)cyclobutyl)phenyl)-7-((cyclohexylamino)methyl)-3,3-dimethyl-2,3-dihydrofuro[3,2-b]pyridine-5-carboxamide

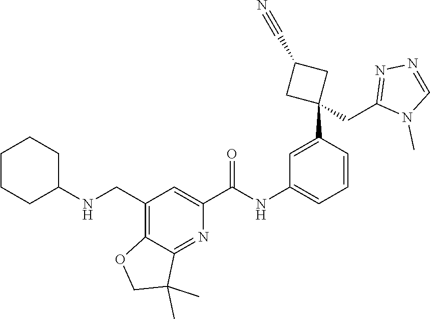 [SMILES: Cn1cnnc1C[C@]1(c2cccc(NC(=O)c3cc(CNC4CCCCC4)c4c(n3)C(C)(C)CO4)c2)C[C@H](C#N)C1]

### Step A: N-(3-((1r, 3r)-3-cyano-1-((4-methyl-4H-1,2,4-triazol-3-yl)methyl)cyclobutyl)phenyl)-7-formyl-3,3-dimethyl-2,3-dihydrofuro[3,2-b]pyridine-5-carboxamide

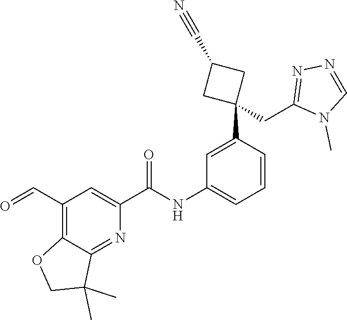 [SMILES: Cn1cnnc1C[C@]1(c2cccc(NC(=O)c3cc(C=O)c4c(n3)C(C)(C)CO4)c2)C[C@H](C#N)C1]

To a solution of 7-formyl-3,3-dimethyl-2,3-dihydrofuro[3,2-b]pyridine-5-carboxylic acid (423 mg, 1.914 mmol), (1r, 3r)-3-(3-aminophenyl)-3-((4-methyl-4H-1,2,4-triazol-3-yl)methyl)cyclobutane-1-carbonitrile (511 mg, 1.914 mmol), DIPEA (494 mg, 3.828 mmol) in DMF (35 mL) was added HATU (1092 mg, 2.871 mmol). The reaction mixture was stirred for 12 hrs at 25° C. After completed, the reaction was quenched with aq NH₄Cl (10 mL) and extracted with EA (2×50 mL), dried over Na₂SO₄ and concentrated under vacuum. The residue was purified by combiflash (DCM:MeOH=20:1) to give the title compound (510 mg, 57%). MS: M/e 471 (M+1)⁺

### Step B: N-(3-((1r, 3r)-3-cyano-1-((4-methyl-4H-1,2,4-triazol-3-yl)methyl)cyclobutyl)phenyl)-7-((cyclohexylamino)methyl)-3,3-dimethyl-2,3-dihydrofuro[3,2-b]pyridine-5-carboxamide

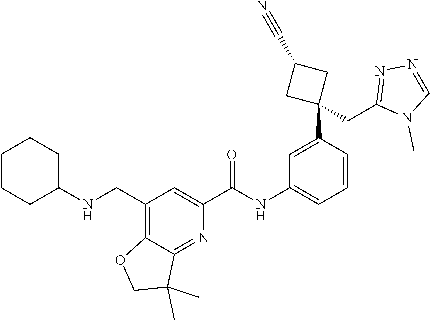 [SMILES: Cn1cnnc1C[C@]1(c2cccc(NC(=O)c3cc(CNC4CCCCC4)c4c(n3)C(C)(C)CO4)c2)C[C@H](C#N)C1]

To a solution of N-(3-((1r, 3r)-3-cyano-1-((4-methyl-4H-1,2,4-triazol-3-yl)methyl)cyclobutyl)phenyl)-7-formyl-3,3-dimethyl-2,3-dihydrofuro[3,2-b]pyridine-5-carboxamide (60 mg, 0.128 mmol), cyclohexanamine hydrochloride (35 mg, 0.255 mmol) and Et₃N (26 mg. 0.255 mmol) in DCM (1 ml) was stirred at room temperature for 1 hour, then added NaBH(OAc)₃ (54 mg, 0.255 mmol). The mixture solution was stirred at 25° C. for 12 hours. The reaction mixture was concentrated under reduced pressure. The crude product was purified by Prep-HPLC to give the title compound (28 mg, 44%). MS: M/e 554 (M+1)⁺. ¹H NMR (400 MHz, DMSO-d₆) δ 10.12 (s, 1H), 8.18 (s, 1H), 8.08 (s, 1H), 7.77 (d, J=8.2 Hz, 1H), 7.53 (s, 1H), 7.26 (t, J=7.9 Hz, 1H), 6.68 (d, J=7.7 Hz, 1H), 4.49 (s, 2H), 3.76 (s, 2H), 3.28 (s, 2H), 3.24 (dd, J=16.4, 7.6 Hz, 1H), 2.91-2.82 (m, 4H), 2.80 (s, 3H), 2.45-2.35 (m, 1H), 1.85 (d, J=10.6 Hz, 2H), 1.68 (dd, J=9.0, 3.4 Hz, 2H), 1.55 (d, J=8.8 Hz, 1H), 1.41 (s, 6H), 1.30-0.99 (m, 6H) ppm.

### Compound C49: 7-(((cyclobutylmethyl)amino)methyl)-3,3-dimethyl-N-(3-((1s, 3s)-3-methyl-1-(4-methyl-4H-1,2,4-triazol-3-yl)cyclobutyl)phenyl)-2,3-dihydro-1H-pyrrolo[3,2-b]pyridine-5-carboxamide

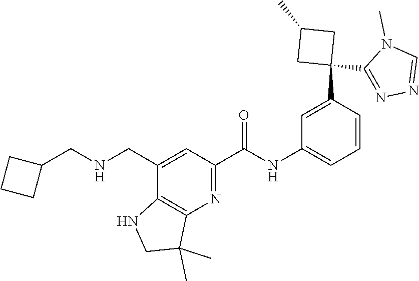 [SMILES: Cn1cnnc1[C@]1(c2cccc(NC(=O)c3cc(CNCC4CCC4)c4c(n3)C(C)(C)CN4)c2)C[C@H](C)C1]

The compound C49 was synthesized starting from the corresponding starting materials according the similar procedures described as those of Compound C46 to give the product (32.9 mg, 44.80%). MS: M/e 514 (M+1)⁺. ¹H NMR (300 MHz, DMSO-d₆) δ 10.03 (s, 1H), 8.29 (s, 1H), 7.87-7.77 (m, 2H), 7.72 (m, 1H), 7.33 (m, 1H), 6.98 (m, 1H), 6.42 (s, 1H), 3.62 (s, 2H), 3.42 (m, 2H), 3.19 (s, 3H), 2.83 (m, 2H), 2.58-2.50 (m, 5H), 2.48-2.35 (m, 1H), 2.00 (m, 2H), 1.88-1.74 (m, 2H), 1.66 (m, 2H), 1.34 (s, 6H), 1.09 (m, 3H).

### Compound C50:7′-(((3R,4S)-4-fluoro-3-methylpiperidin-1-yl)methyl)-N-(3-((1s,3S)-3-methyl-1-(4-methyl-4H-1,2,4-triazol-3-yl)cyclobutyl)phenyl)-1′,2′-dihydrospiro[cyclopropane-1,3′-pyrrolo[3,2-b]pyridine]-5′-carboxamide

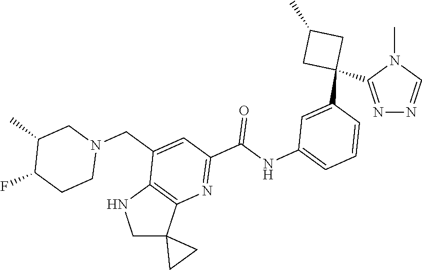 [SMILES: C[C@@H]1CN(Cc2cc(C(=O)Nc3cccc([C@]4(c5nncn5C)C[C@@H](C)C4)c3)nc3c2NCC32CC2)CC[C@@H]1F]

### Step A: 7′-formyl-N-(3-((1s, 3s)-3-methyl-1-(4-methyl-4H-1,2,4-triazol-3-yl)cyclo butyl)phenyl)-1′,2′-dihydrospiro[cyclopropane-1,3′-pyrrolo[3,2-b]pyridine]-5′-carboxamide

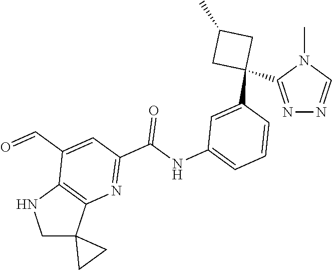 [SMILES: Cn1cnnc1[C@]1(c2cccc(NC(=O)c3cc(C=O)c4c(n3)C3(CC3)CN4)c2)C[C@H](C)C1]

To a solution of 7′-formyl-1′,2′-dihydrospiro[cyclopropane-1,3′-pyrrolo[3,2b]pyridine]-5′-carboxylic acid (40 mg, 0.18 mmol), 3-((1s, 3s)-3-methyl-1-(4-methyl-4H-1,2,4-triazol-3-yl)cyclobutyl)aniline (65 mg, 0.27 mmol) and DIPEA (46 mg, 0.36 mmol) was added HATU (103 mg, 0.27 mmol) at RT. The reaction mixture was stirred at RT for 2 hours. The reaction was quenched by H₂O (10 mL) and extracted with DCM (20 mL×2). The organic phases were concentrated and purified by CombiFlash (C18, ACN: H₂O=40%) to give the desired product (50 mg, 63%). MS: M/e 443 (M+1)⁺.

### Step B: 7′-(((3R,4S)-4-fluoro-3-methylpiperidin-1-yl)methyl)-N-(3-((1s,3S)-3-methyl-1-(4-methyl-4H-1,2,4-triazol-3-yl)cyclobutyl)phenyl)-1′,2′dihydrospiro[cyclopropane-1,3′-pyrrolo[3,2-b]pyridine]-5′-carboxamide

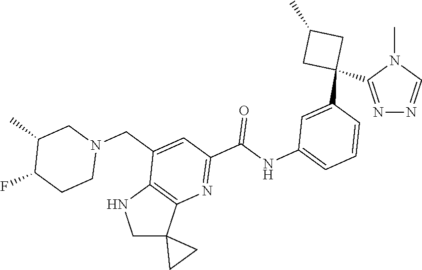 [SMILES: C[C@@H]1CN(Cc2cc(C(=O)Nc3cccc([C@]4(c5nncn5C)C[C@@H](C)C4)c3)nc3c2NCC32CC2)CC[C@@H]1F]

A mixture of 7′-formyl-N-(3-((1s, 3s)-3-methyl-1-(4-methyl-4H-1,2,4-triazol-3-yl)cyclobutyl)phenyl)-1′,2′-dihydrospiro[cyclopropane-1,3′-pyrrolo[3,2-b]pyridine]-5′-carboxamide (30 mg, 0.07 mmol), TEA (14 mg, 0.14 mmol) and (3R,4S)-4-fluoro-3-methylpiperidine hydrochloride (21 mg, 0.14 mmol) was stirred at RT for 30 mins. Then Sodium triacetoxyborohydride (30 mg, 0.14 mmol) was added. The resulting mixture was stirred at RT for overnight. The reaction was quenched by H₂O (5 mL) and extracted with DCM (10 mL×2). The organic phases were concentrated and purified by prep-TLC (DCM:MeOH=85%) to give the desired product (5.2 mg, 14%). ¹H NMR (400 MHz, DMSO-d₆) δ 9.88 (s, 1H), 8.28 (s, 1H), 7.82-7.61 (m, 3H), 7.31-7.29 (m, 1H), 6.97-6.96 (m, 1H), 6.52-6.46 (m, 1H), 4.70-4.60 (m, 1H), 3.82-3.74 (m, 2H), 3.39 (s, 2H), 3.15 (s, 3H), 2.82-2.81 (m, 2H), 2.51 (s, 3H), 2.21-1.75 (m, 5H), 1.34-1.30 (m, 2H), 1.09-1.03 (m, 6H), 0.97-0.84 (m, 4H) ppm. MS: M/e 544 (M+1)⁺.

### Compound C51: N-(3-((1s,3R)-3-methyl-1-(4-methyl-4H-1,2,4-triazol-3-yl)cyclobutyl)phenyl)-7′-(((S)-3-methylpiperidin-1-yl)methyl)-1′,2′-dihydrospiro[cyclopropane-1,3′-pyrrolo[3,2-b]pyridine]-5′-carboxamide

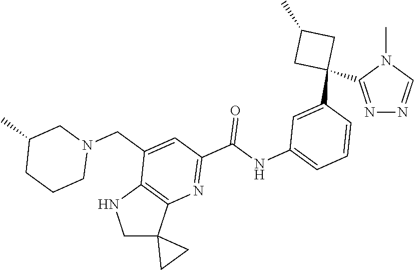 [SMILES: C[C@H]1CCCN(Cc2cc(C(=O)Nc3cccc([C@]4(c5nncn5C)C[C@@H](C)C4)c3)nc3c2NCC32CC2)C1]

To solution of (R)-7′-((3-methylpiperidin-1-yl)methyl)-1′,2′-dihydrospiro[cyclo propane-1,3′-pyrrolo[3,2-b]pyridine]-5′-carboxylic acid (15 mg, 0.05 mmol) and 3-((1r, 3r)-3-methyl-1-((4-methyl-4H-1,2,4-triazol-3-yl)methyl)cyclobutyl)aniline (15 mg, 0.06 mmol) and DIEA (13 mg, 0.1 mmol) in DMF (1 mL) were added HATU (27 mg, 0.07 mmol). The reaction mixture was stirred at RT for 2 hours. The reaction was quenched by H₂O (5 mL), extracted with DCM (10 mL×2). The organic phases were concentrated and purified by prep-TLC (DCM:MeOH=85%) to give the desired product (10.45 mg, 39%). ¹H NMR (500 MHz, DMSO-d₆) δ 9.88 (s, 1H), 8.28 (s, 1H), 7.83 (s, 1H), 7.66-7.60 (m, 2H), 7.31 (t, J=7.9 Hz, 1H), 6.96 (d, J=8.3 Hz, 1H), 6.44 (s, 1H), 3.74 (s, 2H), 3.34 (s, 2H), 3.17 (s, 3H), 2.82-2.81 (m, 2H), 2.72-2.71 (m, 2H), 2.53-2.51 (m, 3H), 1.90-1.88 (m, 1H), 1.61-1.49 (m, 5H), 1.36-1.33 (m, 2H), 1.10-1.07 (m, 3H), 1.04-1.01 (m, 2H), 0.96-0.94 (m, 1H), 0.85-0.80 (m, 3H) ppm. MS: M/e 540 (M+1)⁺.

### Compound C52: 4-{[(cyclobutylmethyl)amino]methyl}-7,7-difluoro-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide

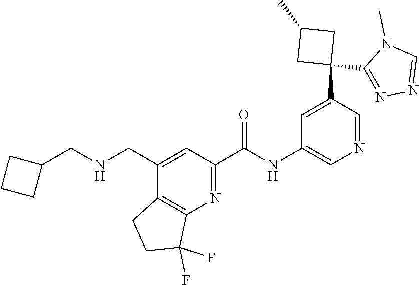 [SMILES: Cn1cnnc1[C@]1(c2cncc(NC(=O)c3cc(CNCC4CCC4)c4c(n3)C(F)(F)CC4)c2)C[C@H](C)C1]

A solution of 4-(chloromethyl)-7,7-difluoro-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide (8 mg, 0.012 mmol, 1 equiv, 73.3%), 1-cyclobutylmethanamine hydrochloride (5 mg, 0.039 mmol, 3.15 equiv, 95%), NaI (2 mg, 0.013 mmol, 1.02 equiv, 95%) and K₂CO₃ (4 mg, 0.027 mmol, 2.22 equiv, 95%) in MeCN was stirred for 3 hrs at 70° C. under nitrogen atmosphere. The resulting mixture was concentrated under reduced pressure. The crude product was purified by Prep-HPLC with the following conditions (2 #SHIMADZU (HPLC-01)): Column, YMC-Actus Triart C18 ExRS, 20*250 mm, 5 um; mobile phase, 10 mmol NH₄HCO₃+0.05% NH₃H₂O and ACN (38% ACN up to 68% in 8 min); This resulted in 4-{[(cyclobutylmethyl)amino]methyl}-7,7-difluoro-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide (1.3 mg, 19.94%). MS: M/e 522 (M+1)⁺. ¹H NMR (300 MHz, DMSO-d₆) δ 10.69 (s, 1H), 9.08 (d, J=2.2 Hz, 1H), 8.46-8.15 (m, 4H), 3.84 (s, 2H), 3.22 (s, 3H), 3.09 (s, 2H), 2.92-2.84 (m, 2H), 2.79-2.63 (m, 2H), 2.64-2.54 (m, 5H), 2.49-2.39 (m, 1H), 2.10-1.95 (m, 2H), 1.91-1.77 (m, 2H), 1.75-1.58 (m, 2H), 1.10 (d, J=4.9 Hz, 3H).

### Compound C53: 7,7-difluoro-4-{[(2-methylpropyl)amino]methyl}-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide

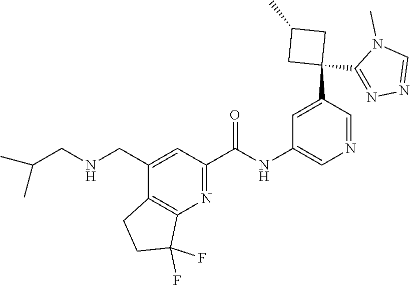 [SMILES: CC(C)CNCc1cc(C(=O)Nc2cncc([C@]3(c4nncn4C)C[C@@H](C)C3)c2)nc2c1CCC2(F)F]

A solution of 4-(chloromethyl)-7,7-difluoro-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide (8 mg, 0.012 mmol, 1 equiv, 73.3%), isobutylamine (2 mg, 0.026 mmol, 2.10 equiv, 95%), NaI (4 mg, 0.025 mmol, 2.04 equiv, 95%) and K₂CO₃ (4 mg, 0.027 mmol, 2.22 equiv, 95%) in MeCN (2 mL) was stirred for 3 hrs at 70° C. under nitrogen atmosphere. The mixture was allowed to cool down to room temperature. The resulting mixture was concentrated under reduced pressure. The crude product was purified by Prep-HPLC with the following conditions (2 #SHIMADZU (HPLC-01)): Column, YMC-Actus Triart C18 ExRS, 20*250 mm, 5 um; mobile phase, 10 mmol NH₄HCO₃+0.05% NH₃H₂O and ACN (38% ACN up to 68% in 8 min); This resulted in 7,7-difluoro-4-{[(2-methylpropyl)amino]methyl}-N-{5-[(1r, 3s)-3-methyl-1-(4-methyl-1,2,4-triazol-3-yl)cyclobutyl]pyridin-3-yl}-5H,6H-cyclopenta[b]pyridine-2-carboxamide (1.0 mg, 15.59%). MS: M/e 510 (M+1)⁺. ¹H NMR (300 MHz, DMSO-d₆) δ 10.70 (s, 1H), 9.12-9.05 (m, 1H), 8.44-8.06 (m, 4H), 3.86 (d, J=2.4 Hz, 2H), 3.23 (d, J=2.3 Hz, 3H), 3.11 (s, 2H), 2.89 (s, 2H), 2.78-2.67 (m, 3H), 2.62-2.54 (m, 3H), 2.38 (d, J=6.8, 2.4 Hz, 2H), 1.81-1.66 (m, 1H), 1.13-1.08 (m, 3H), 0.95-0.86 (m, 6H).

### Compound C54: N-(3-((1s, 3s)-3-(cyanomethyl)-1-(4-methyl-4H-1,2,4-triazol-3-yl)cyclobutyl)phenyl)-4-((cycloheptylamino)methyl)-7,7-dimethyl-6,7-dihydro-5H-cyclopenta[b]pyridine-2-carboxamide

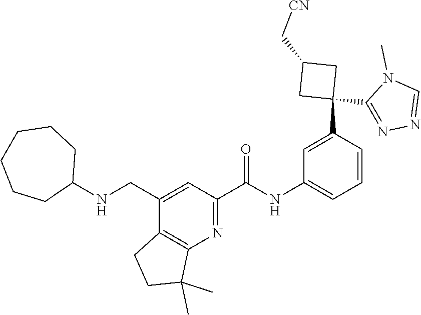 [SMILES: Cn1cnnc1[C@]1(c2cccc(NC(=O)c3cc(CNC4CCCCCC4)c4c(n3)C(C)(C)CC4)c2)C[C@H](CC#N)C1]

### Step 1: (2-((3-((1s, 3s)-3-(cyanomethyl)-1-(4-methyl-4H-1,2,4-triazol-3-yl)cyclobutyl)phenyl)carbamoyl)-7,7-dimethyl-6,7-dihydro-5H-cyclopenta[b]pyridin-4-yl)methyl methanesulfonate and 4-(chloromethyl)-N-(3-((1s, 3s)-3-(cyanomethyl)-1-(4-methyl-4H-1,2,4-triazol-3-yl)cyclobutyl)phenyl)-7,7-dimethyl-6,7-dihydro-5H-cyclopenta[b]pyridine-2-carboxamide mixture

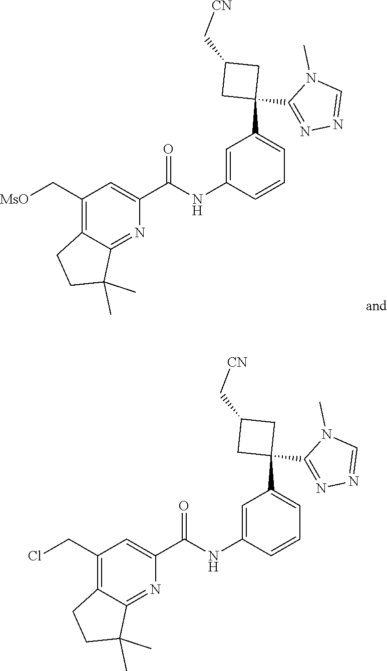 [SMILES: Cn1cnnc1[C@]1(c2cccc(NC(=O)c3cc(COS(C)(=O)=O)c4c(n3)C(C)(C)CC4)c2)C[C@H](CC#N)C1 ; Cn1cnnc1[C@]1(c2cccc(NC(=O)c3cc(CCl)c4c(n3)C(C)(C)CC4)c2)C[C@H](CC#N)C1]

To a solution of N-(3-((1s, 3s)-3-(cyanomethyl)-1-(4-methyl-4H-1,2,4-triazol-3-yl)cyclobutyl)phenyl)-4-(hydroxymethyl)-7,7-dimethyl-6,7-dihydro-5H-cyclopenta[b]pyridine-2-carboxamide (0.12 g, 255.01 umol), TEA (412.88 mg, 4.08 mmol, 567.92 uL) in DMF (3 mL) was added MsCl (140.22 mg, 1.22 mmol, 94.74 uL) at 0° C. Then the mixture was stirred at 25° C. for 2 hrs The mixture was poured into 40 mL saturated ammonium chloride aqueous solution, extracted with Dichloromethane (30 mL*2), washed with brine(20 mL*2), the combined organic layers were dried over Na₂SO₄, filtered and concentrated under reduced pressure to give a residue. Compound (2-((3-((1s, 3s)-3-(cyanomethyl)-1-(4-methyl-4H-1,2,4-triazol-3-yl)cyclobutyl)phenyl)carbamoyl)-7,7-dimethyl-6,7-dihydro-5H-cyclopenta[b]pyridin-4-yl)methyl methanesulfonate and 4-(chloromethyl)-N-(3-((1s, 3s)-3-(cyanomethyl)-1-(4-methyl-4H-1,2,4-triazol-3-yl)cyclobutyl)phenyl)-7,7-dimethyl-6,7-dihydro-5H-cyclopenta[b]pyridine-2-carboxamide as a mixture (0.12 g, crude) was obtained. MS: M/e 549 (M+1)⁺.

### Step 2: N-(3-((1s, 3s)-3-(cyanomethyl)-1-(4-methyl-4H-1,2,4-triazol-3-yl)cyclobutyl)phenyl)-4-((cycloheptylamino)methyl)-7,7-dimethyl-6,7-dihydro-5H-cyclopenta[b]pyridine-2-carboxamide

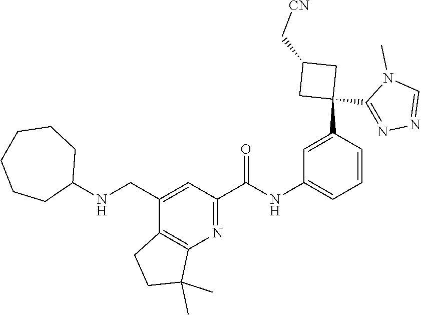 [SMILES: Cn1cnnc1[C@]1(c2cccc(NC(=O)c3cc(CNC4CCCCCC4)c4c(n3)C(C)(C)CC4)c2)C[C@H](CC#N)C1]

The crude product of step 1 (0.025 g, 51.12 umol), cycloheptanamine (57.87 mg, 511.24 umol, 65.10 uL), Cs₂CO₃ (33.31 mg, 102.25 umol), DMF (1.5 mL) was stirred at 25° C. for 12 hrs. The mixture was filtered, the filtrate was concentrated in vacuo. The crude was purified by Prep-HPLC(column: Waters Xbridge BEH C18 100*30 mm*10 um; mobile phase: [water (NH₄HCO₃)-ACN]; B %: 45%-75%, 8 min). to give the title product (14.79 mg, 26.14 umol, 51.14% yield). MS: M/e 566 (M+1)⁺; ¹H NMR (400 MHz, DMSO-d₆) δ ppm 10.32 (s, 1H), 8.31 (s, 1H), 8.01 (s, 1H), 7.87 (s, 1H), 7.76-7.81 (m, 1H), 7.39 (t, J=7.94 Hz, 1H), 7.08 (d, J=8.38 Hz, 1H), 3.72 (s, 2H), 3.21 (s, 3H), 2.89 (d, J=7.06 Hz, 4H), 2.65-2.79 (m, 5H), 2.56-2.63 (m, 2H), 1.95-2.00 (m, 2H), 1.75-1.84 (m, 2H), 1.57-1.65 (m, 2H), 1.47-1.53 (m, 4H), 1.35-1.43 (m, 3H), 1.32 (s, 6H).

### Compound C55: 4-(((3R,4S)-4-fluoro-3-methylpiperidin-1-yl)methyl)-7,7-dimethyl-N-(3-((1s,3S)-3-methyl-1-(4-methyl-4H-1,2,4-triazol-3-yl)cyclobutyl)phenyl)-6,7-dihydro-5H-cyclopenta[b]pyridine-2-carboxamide

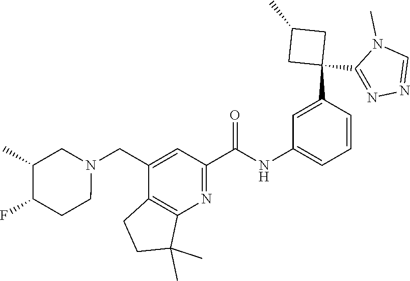 [SMILES: C[C@@H]1CN(Cc2cc(C(=O)Nc3cccc([C@]4(c5nncn5C)C[C@@H](C)C4)c3)nc3c2CCC3(C)C)CC[C@@H]1F]

### Step 1: lithium 4-(hydroxymethyl)-7,7-dimethyl-6,7-dihydro-5H-cyclopenta[b]pyridine-2-carboxylate

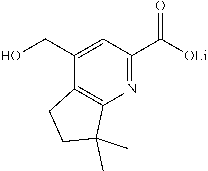

To a solution of methyl 4-(hydroxymethyl)-7,7-dimethyl-6,7-dihydro-5H-cyclopenta[b]pyridine-2-carboxylate (1.6 g, 6.80 mmol) in MeOH (5 mL), THF (5 mL) and H₂O (5 mL) was added LiOH·H₂O (342.42 mg, 8.16 mmol). The mixture was stirred at 25° C. for 4 hrs. The reaction mixture was re-crystallized from water, dried by hyophilization to give the product (1.5 g, crude) was used into the next step without further purification. MS: M/e 222 (M+1)⁺; ¹H NMR (400 MHz, DMSO-d₆) δ=7.95 (s, 1H), 5.47 (br s, 1H), 4.51 (br s, 2H), 2.75 (brt, J=8.0 Hz, 2H), 1.89 (br t, J=8.0 Hz, 2H), 1.17 (s, 6H).

### Step 2: 4-(hydroxymethyl)-7,7-dimethyl-N-(3-((1s, 3s)-3-methyl-1-(4-methyl-4H-1,2,4-triazol-3-yl)cyclobutyl)phenyl)-6,7-dihydro-5H-cyclopenta[b]pyridine-2-carboxamide

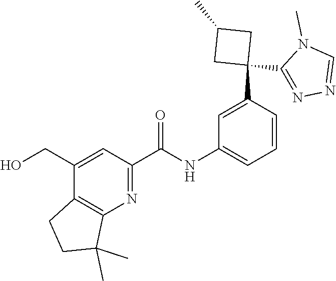 [SMILES: Cn1cnnc1[C@]1(c2cccc(NC(=O)c3cc(CO)c4c(n3)C(C)(C)CC4)c2)C[C@H](C)C1]

To a solution of lithium 4-(hydroxymethyl)-7,7-dimethyl-6,7-dihydro-5H-cyclopenta[b]pyridine-2-carboxylate (351.58 mg, 1.24 mmol, 80% purity) in DMF (2 mL) was added HATU (564.89 mg, 1.49 mmol), DIEA (320.02 mg, 2.48 mmol) and 3-((1s, 3s)-3-methyl-1-(4-methyl-4H-1,2,4-triazol-3-yl)cyclobutyl)aniline (300 mg, 1.24 mmol). The mixture was stirred at 25° C. for 2 hrs. The reaction mixture was quenched by addition H₂O 5 mL at 20° C., and then extracted with DCM 30 mL (10 mL*3). The combined organic layers were washed with brine 30 mL (10 mL*3), dried over Na₂SO₄, filtered and concentrated under reduced pressure to give a residue. The residue was purified by Prep-HPLC(column: Phenomenex C18 80*40 mm*3 um; mobile phase: [water (NH₄HCO₃)-ACN]; B %: 20%-50%, 8 min) to give a product (100 mg, 18.13% yield). MS: M/e 446 (M+1)⁺; ¹H NMR (400 MHz, DMSO-d₆) δ=10.32 (s, 1H), 8.29 (s, 1H), 8.02 (s, 1H), 7.88-7.82 (m, 1H), 7.81-7.74 (m, 1H), 7.37 (t, J=8.0 Hz, 1H), 7.05 (d, J=8.0 Hz, 1H), 5.49 (t, J=8.0 Hz, 1H), 4.57 (d, J=8.0 Hz, 2H), 3.19 (s, 3H), 2.84 (br t, J=8.0 Hz, 4H), 2.54 (br d, J=8.0 Hz, 3H), 2.00 (t, J=8.0 Hz, 2H), 1.33 (s, 6H), 1.09 (d, J=4.0 Hz, 3H).

### Step 3: 4-(chloromethyl)-7,7-dimethyl-N-(3-((1s, 3s)-3-methyl-1-(4-methyl-4H-1,2,4-triazol-3-yl)cyclobutyl)phenyl)-6,7-dihydro-5H-cyclopenta[b]pyridine-2-carboxamide

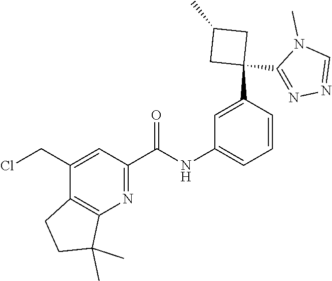 [SMILES: Cn1cnnc1[C@]1(c2cccc(NC(=O)c3cc(CCl)c4c(n3)C(C)(C)CC4)c2)C[C@H](C)C1]

In a mixture of 4-(hydroxymethyl)-7,7-dimethyl-N-(3-((1s, 3s)-3-methyl-1-(4-methyl-4H-1,2,4-triazol-3-yl)cyclobutyl)phenyl)-6,7-dihydro-5H-cyclopenta[b]pyridine-2-carboxamide (100 mg, 224.44 mol) DMF (3 mL) TEA (181.69 mg, 1.80 mmol) was added MsCl (123.41 mg, 1.08 mmol) at 0° C., then the mixture was warmed to 25° C. for 1 hr. Then MsCl (123.41 mg, 1.08 mmol) and TEA (181.69 mg, 1.80 mmol) was added dropwise at 0° C. The resulting mixture was stirred at 25° C. for 1 hr. The mixture was poured into 5 ml H₂O, extracted with Dichloromethane (5 mL*2), washed with brine (5 mL*2), the combined organic layers were dried over Na₂SO₄, filtered and concentrated under reduced pressure to give the product (100 mg, crude) was used into the next step without further purification. MS: M/e 464 (M+1)⁺.

### Step 4: 4-(((3R,4S)-4-fluoro-3-methylpiperidin-1-yl)methyl)-7,7-dimethyl-N-(3-((1s,3S)-3-methyl-1-(4-methyl-4H-1,2,4-triazol-3-yl)cyclobutyl)phenyl)-6,7-dihydro-5H-cyclopenta[b]pyridine-2-carboxamide

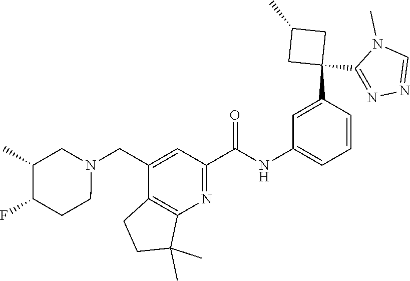 [SMILES: C[C@@H]1CN(Cc2cc(C(=O)Nc3cccc([C@]4(c5nncn5C)C[C@@H](C)C4)c3)nc3c2CCC3(C)C)CC[C@@H]1F]

To a solution of (3R,4S)-4-fluoro-3-methylpiperidine hydrochloride (49.66 mg, 323.28 mol) in DMF (1 mL) was added TEA (16.36 mg, 161.64 mol). The mixture was stirred at 25° C. for 2 hrs. Then 4-(chloromethyl)-7,7-dimethyl-N-(3-((1s, 3s)-3-methyl-1-(4-methyl-4H-1,2,4-triazol-3-yl)cyclobutyl)phenyl)-6,7-dihydro-5H-cyclopenta[b]pyridine-2-carboxamide (25 mg, 53.88 mol) and Cs₂CO₃ (35.11 mg, 107.76 mol) was added at 25° C. The resulting mixture was stirred at 25° C. for 2 hrs. The mixture was filtered, the filtrate was concentrated in vacuo, which was purified by Prep-HPLC (column: Waters Xbridge Prep OBD C18 150*40 mm*10 um; mobile phase: [water (NH₄HCO₃)-ACN]; B %: 40%-80%, 8 min) to give the product (3.71 mg, 12.64% yield). MS: M/e 545 (M+1)⁺. ¹H NMR (400 MHz, DMSO-d₆) δ=10.31 (s, 1H), 8.29 (s, 1H), 7.92 (s, 1H), 7.88 (s, 1H), 7.72 (dd, J=0.8, 8.0 Hz, 1H), 7.36 (t, J=8.0 Hz, 1H), 7.05 (d, J=8.0 Hz, 1H), 4.80-4.50 (m, 1H), 3.53 (s, 2H), 3.18 (s, 3H), 2.91 (t, J=8.0 Hz, 2H), 2.87-2.76 (m, 2H), 2.54 (br d, J=8.0 Hz, 4H), 2.47 (br d, J=4.0 Hz, 1H), 2.27 (br t, J=12.0 Hz, 1H), 2.14-1.64 (m, 6H), 1.32 (s, 6H), 1.08 (d, J=4.0 Hz, 3H), 0.90 (d, J=8.0 Hz, 3H).

### Compound C56: 4-(((cyclobutylmethyl)amino)methyl)-7,7-difluoro-N-(3-((1s, 3s)-3-methyl-1-(4-methyl-4H-1,2,4-triazol-3-yl)cyclobutyl)phenyl)-6,7-dihydro-5H-cyclopenta[b]pyridine-2-carboxamide

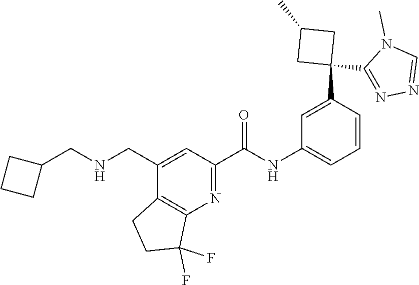 [SMILES: Cn1cnnc1[C@]1(c2cccc(NC(=O)c3cc(CNCC4CCC4)c4c(n3)C(F)(F)CC4)c2)C[C@H](C)C1]

The compound C56 was synthesized starting from the corresponding starting materials according the similar procedures described as those of Compound C53 to give the product (6.31 mg, 25.77% yield). MS: M/e 521 (M+1)⁺. ¹H NMR (400 MHz, DMSO-d₆) δ=10.45-10.40 (m, 1H), 8.31 (s, 1H), 8.29 (s, 1H), 7.91-7.84 (m, 2H), 7.40-7.32 (m, 1H), 7.02 (d, J=7.6 Hz, 1H), 3.82 (s, 2H), 3.18 (s, 3H), 3.08 (t, J=6.4 Hz, 2H), 2.87-2.79 (m, 2H), 2.78-2.65 (m, 2H), 2.58-2.51 (m, 5H), 2.48-2.40 (m, 1H), 2.05-1.96 (m, 2H), 1.91-1.75 (m, 2H), 1.72-1.60 (m, 2H), 1.08 (d, J=6.0 Hz, 3H).

### Compound C57: 4-(((cyclobutylmethyl)amino)methyl)-7,7-dimethyl-N-(3-((1s, 3s)-3-methyl-1-(4-methyl-4H-1,2,4-triazol-3-yl)cyclobutyl)phenyl)-6,7-dihydro-5H-cyclopenta[b]pyridine-2-carboxamide

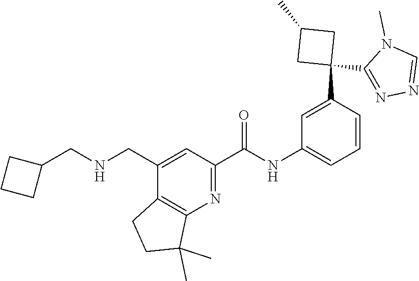 [SMILES: Cn1cnnc1[C@]1(c2cccc(NC(=O)c3cc(CNCC4CCC4)c4c(n3)C(C)(C)CC4)c2)C[C@H](C)C1]

The compound C57 was synthesized starting from the corresponding starting materials according the similar procedures described as those of Compound C55 to give the product (4.49 mg, 13.55% yield). MS: M/e 513 (M+1)⁺; ¹H NMR (400 MHz, DMSO-d₆) δ=10.31 (s, 1H), 8.29 (s, 1H), 8.00 (s, 1H), 7.86 (s, 1H), 7.79-7.68 (m, 1H), 7.36 (t, J=8.0 Hz, 1H), 7.04 (d, J=8.0 Hz, 1H), 3.72 (s, 2H), 3.19 (s, 3H), 2.98-2.75 (m, 4H), 2.62-2.52 (m, 5H), 2.43 (td, J=8.0, 16.0 Hz, 1H), 2.06-1.93 (m, 4H), 1.91-1.75 (m, 2H), 1.73-1.58 (m, 2H), 1.32 (s, 6H), 1.08 (d, J=4.0 Hz, 3H).

### Compound C58: 7-(((3R,4S)-4-fluoro-3-methylpiperidin-1-yl)methyl)-3,3-dimethyl-N-(5-((1s,3S)-3-methyl-1-(4-methyl-4H-1,2,4-triazol-3-yl)cyclobutyl)pyridin-3-yl)-2,3-dihydro-1H-pyrrolo[3,2-b]pyridine-5-carboxamide

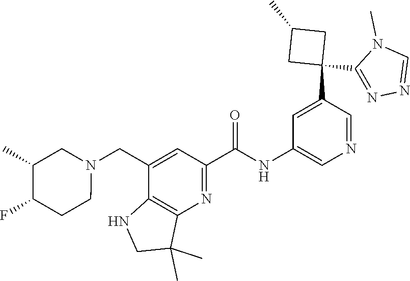 [SMILES: C[C@@H]1CN(Cc2cc(C(=O)Nc3cncc([C@]4(c5nncn5C)C[C@@H](C)C4)c3)nc3c2NCC3(C)C)CC[C@@H]1F]

### Step 1: 7-formyl-3,3-dimethyl-N-(5-((1s, 3s)-3-methyl-1-(4-methyl-4H-1,2,4-triazol-3-yl)cyclobutyl)pyridin-3-yl)-2,3-dihydro-1H-pyrrolo[3,2-b]pyridine-5-carboxamide

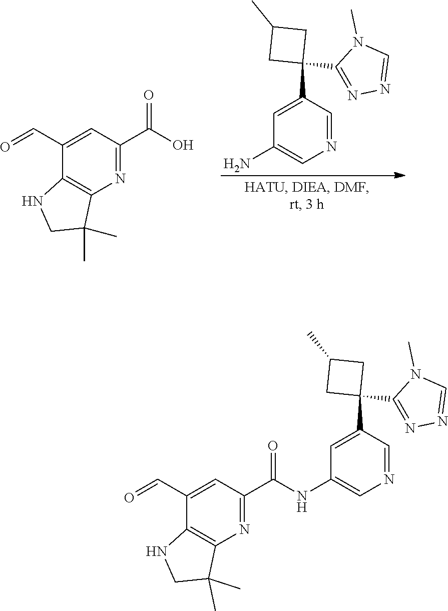

To a stirred solution of 7-formyl-3,3-dimethyl-1H,2H-pyrrolo[3,2-b]pyridine-5-carboxylic acid (40 mg, 0.173 mmol, 1 equiv, 95%) and DIEA (93.90 mg, 0.692 mmol, 4 equiv, 95%) in DMF (3 ml) were added HATU (103.59 mg, 0.259 mmol, 1.5 equiv, 95%) and 5-[(1r, 3s)-3-methyl-1-(4-methyl-1,2,4-triazol-3-yl)cyclobutyl]pyridin-3-amine (44.19 mg, 0.173 mmol, 1 equiv, 95%) at room temperature under nitrogen atmosphere. The resulting mixture was stirred for additional 3 hrs at room temperature. The resulting mixture was concentrated under vacuum. The residue was purified by reversed-phase flash chromatography with the following conditions: column, C18 silica gel; mobile phase, MeCN in Water (10 mmol/L NH₄HCO₃), 0% to 100% gradient in 30 min; This resulted in 7-formyl-3,3-dimethyl-N-{5-[(1r, 3s)-3-methyl-1-(4-methyl-1,2,4-triazol-3-yl)cyclobutyl]pyridin-3-yl}-1H,2H-pyrrolo[3,2-b]pyridine-5-carboxamide (70 mg, 91.06%). MS: M/e 446 (M+1)⁺.

### Step 2: 7-(((3R,4S)-4-fluoro-3-methylpiperidin-1-yl)methyl)-3,3-dimethyl-N-(5-((1s,3S)-3-methyl-1-(4-methyl-4H-1,2,4-triazol-3-yl)cyclobutyl)pyridin-3-yl)-2,3-dihydro-1H-pyrrolo[3,2-b]pyridine-5-carboxamide

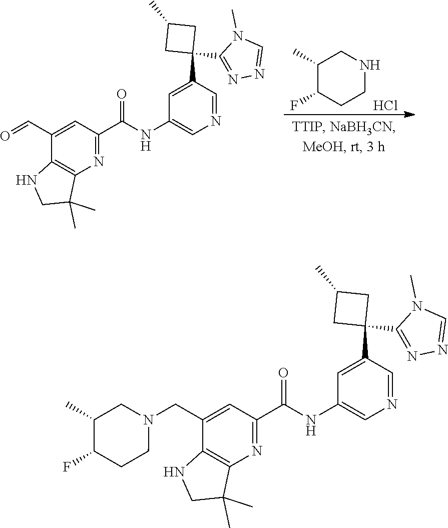

To a stirred solution of 7-formyl-3,3-dimethyl-N-{5-[(1r, 3s)-3-methyl-1-(4-methyl-1,2,4-triazol-3-yl)cyclobutyl]pyridin-3-yl}-1H,2H-pyrrolo[3,2-b]pyridine-5-carboxamide (25 mg, 0.053 mmol, 1 equiv, 95%) and (3R,4S)-4-fluoro-3-methylpiperidine hydrochloride (17.24 mg, 0.106 mmol, 2 equiv, 95%) in MeOH (2 ml) was added tetrakis(propan-2-yloxy)titanium (63.79 mg, 0.212 mmol, 4 equiv, 95%) at room temperature under nitrogen atmosphere. The resulting mixture was stirred for additional 1 h at room temperature. To the above mixture was added NaBH₃CN (7.05 mg, 0.106 mmol, 2 equiv, 95%). The resulting mixture was stirred for additional 1 h at room temperature. The crude product was purified by Chiral-Prep-HPLC with the following conditions (2 #SHIMADZU (HPLC-01)): Column, XBridge Shield RP18 OBD Column, 30*150 mm, 5 μm; mobile phase, 10 mmol NH₄HCO₃+0.05% NH₃H₂O and ACN (32% ACN up to 62% in 8 min); This resulted in 7-{[(3R,4S)-4-fluoro-3-methylpiperidin-1-yl]methyl}-3,3-dimethyl-N-{5-[(1r, 3s)-3-methyl-1-(4-methyl-1,2,4-triazol-3-yl)cyclobutyl]pyridin-3-yl}-1H,2H-pyrrolo[3,2-b]pyridine-5-carboxamide (11.3 mg, 36.87%). MS: M/e 547 (M+1)⁺. ¹H NMR (300 MHz, DMSO-d₆) δ 10.20 (s, 1H), 8.96 (m, 1H), 8.37-8.16 (m, 3H), 7.71 (s, 1H), 6.43 (s, 1H), 4.65 (m, 1H), 3.42 (m, 4H), 3.22 (s, 3H), 2.88 (m, 2H), 2.57 (m, 4H), 2.22 (m, 1H), 2.07-1.82 (m, 4H), 1.72 (m, 1H), 1.35 (s, 6H), 1.09 (m, 3H), 0.90 (m, 3H).

### Compound C59: 4-((((S)-1-cyclobutylethyl)amino)methyl)-7,7-difluoro-N-(3-((1s,3R)-3-methyl-1-(4-methyl-4H-1,2,4-triazol-3-yl)cyclobutyl)phenyl)-6,7-dihydro-5H-cyclopenta[b]pyridine-2-carboxamide

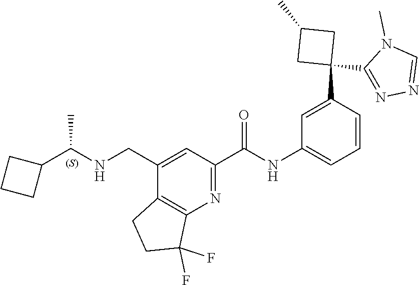

The compound C59 was synthesized starting from the corresponding starting materials according the similar procedures described as those of Compound C53 to give the product (7.61 mg, 25.2% yield). MS: M/e 535 (M+1)⁺. ¹H NMR (400 MHz, DMSO-d₆) δ=10.42 (s, 1H), 8.34 (s, 1H), 8.29 (s, 1H), 7.90-7.84 (m, 2H), 7.35 (t, J=8.0 Hz, 1H), 7.01 (br d, J=7.6 Hz, 1H), 3.91-3.75 (m, 2H), 3.18 (s, 3H), 3.09 (br s, 2H), 2.88-2.65 (m, 5H), 2.57 (br s, 1H), 2.53 (br d, J=8.4 Hz, 2H), 2.27-2.17 (m, 1H), 2.04-1.95 (m, 1H), 1.94-1.85 (m, 1H), 1.83-1.63 (m, 4H), 1.08 (br d, J=5.6 Hz, 3H), 0.92 (d, J=6.0 Hz, 3H)

### Compound C60: 4-((((S)-1-cyclopropylethyl)amino)methyl)-7,7-difluoro-N-(3-((1s,3R)-3-methyl-1-(4-methyl-4H-1,2,4-triazol-3-yl)cyclobutyl)phenyl)-6,7-dihydro-5H-cyclopenta[b]pyridine-2-carboxamide

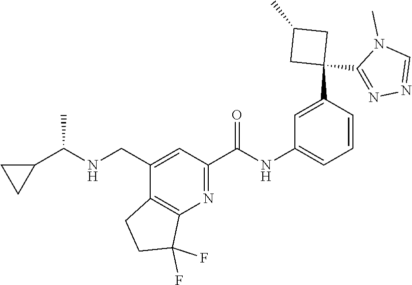 [SMILES: C[C@H](NCc1cc(C(=O)Nc2cccc([C@]3(c4nncn4C)C[C@@H](C)C3)c2)nc2c1CCC2(F)F)C1CC1]

The compound C60 was synthesized starting from the corresponding starting materials according the similar procedures described as those of Compound C53 to give the product (24.63 mg, 47.31 μmol, 44.65% yield). MS: M/e 521 (M+1)⁺. ¹H NMR (400 MHz, DMSO-d₆) δ ppm 10.42 (s, 1H), 8.36 (s, 1H), 8.29 (s, 1H), 7.84-7.91 (m, 2H), 7.35 (t, J=8.20 Hz, 1H), 7.01 (d, J=8.00 Hz, 1H), 3.93-3.98 (m, 1H), 3.91 (d, J=7.62 Hz, 1H), 3.19 (s, 3H), 3.10 (s, 2H), 2.64-2.87 (m, 4H), 2.52-2.56 (m, 2H), 2.33-2.45 (m, 1H), 1.82-2.00 (m, 1H), 1.10 (d, J=12.06 Hz, 6H), 0.69 (d, J=8.12 Hz, 1H), 0.30-0.46 (m, 2H), 0.21 (d, J=9.12 Hz, 1H), −0.02-0.08 (m, 1H).

### Assays

### Biochemical Assays

### Cbl-b or Cbl Biochemical Assay Protocol

Compounds disclosed herein were measured by stabilizing an inactive conformation of Cbl-b/Cbl and preventing the interaction of Cbl-b/Cbl with UbcH5B-Ub (an E2 enzyme labeled with the Bodipy-Fluorescein tagged ubiquitin) in an assay based on Homogeneous Time Resolved Fluorescence. The assay in the absence of a Cbl-b/Cbl inhibitor, where phosphorylation of Cbl-b/Cbl by the Src kinase allows UbcH5B-Ub to bind to Cbl-b/Cbl and brings the Bodipy-Fluorescein tagged ubiquitin in proximity to the streptavidin-terbium bound to the N-terminal biotinylated Avi-tagged Cbl-b/Cbl. The resulting complex produces a FRET signal indicating that Cbl-b/Cbl is active or not subject to inhibition. The assay in the presence of a Cbl-b/Cbl inhibitor, where Cbl-b/Cbl is stabilized an inactive conformation and unable to bind UbcH5B-Ub. The lack of a FRET signal indicates Cbl-b/Cbl is inhibited by the Cbl-b/Cbl inhibitor.

Co-Expressed Cbl-b with BirA Biochemical Assay

Compounds disclosed herein were tested for stabilization of an inactive conformation of Cbl-b (aa36-427, in-house) and preventing the interaction of Cbl-b with UbcH5B-Ub in an assay based on Homogeneous Time Resolved Fluorescence. 0.6 nM recombinant co-expressed Cbl-b with BirA was pre-incubated with a serial dilution of compounds (maximum concentration is 100 μM, 3-fold serially diluted, 16 points) at room temperature for 1.5 hour in an assay buffer containing 50 mM HEPES, pH 7.0, 100 mM NaCl, 0.01% BSA, 1 mM DTT, 0.01% Triton X-100. Then 12 nM in-house UbcH5B-Ub conjugate protein, 120 nM SRC (aa251-536, in-house), 0.5 mM ATP, 5 mM MgCl₂ and Streptavidin-Tb cryptate (Cat: 610SATLB, Cisbio Bioassays) were added to plate and further incubated at room temperature for 2 hours. The HTRF signals (ex337 nm, em520 nm/620 nm) were read on BMG PHERAstar FS instrument. The inhibition percentage of Cbl-b interaction with UbcH5B-Ub in presence of increasing concentrations of compounds was calculated based on the ratio of fluorescence at 520 nm to that at 620 nm. The IC₅₀ for each compound was derived from fitting the data to the four-parameter logistic equation by Dotmatics.

### Biotin-Labelled Cbl-b Biochemical Assay

Compounds disclosed herein were tested for stabilization of an inactive conformation of Cbl-b (aa36-427, in-house) and preventing the interaction of Cbl-b with UbcH5B-Ub in an assay based on Homogeneous Time Resolved Fluorescence. 5.0 nM recombinant biotin labelled Cbl-b was pre-incubated with a serial dilution of compounds (maximum concentration is 100 μM, 3-fold serially diluted, 16 points) at room temperature for 1.5 hour in an assay buffer containing 50 mM HEPES, pH 7.0, 100 mM NaCl, 0.01% BSA, 1 mM DTT, 0.01% Triton X-100. Then 25 nM in-house UbcH5B-Ub conjugate protein, 60 nM SRC (aa251-536, in-house), 0.5 mM ATP, 5 mM MgCl₂ and Streptavidin-Tb cryptate (Cat: 610SATLB, Cisbio Bioassays) were added to plate and further incubated at room temperature for 2 hours. The HTRF signals (ex337 nm, em 520 nm/620 nm) were read on BMG PHERAstar FS instrument. The inhibition percentage of Cbl-b interaction with UbcH5B-Ub in presence of increasing concentrations of compounds was calculated based on the ratio of fluorescence at 520 nm to that at 620 nm. The IC₅₀ for each compound was derived from fitting the data to the four-parameter logistic equation by Dotmatics.

Co-Expressed Cbl with BirA Biochemical Assay

Compounds disclosed herein were tested for stabilization of an inactive conformation of Cbl (aa 49-434, in-house) and preventing the interaction of Cbl-b with UbcH5B-Ub in an assay based on Homogeneous Time Resolved Fluorescence. 0.6 nM recombinant co-expressed Cbl with BirA was pre-incubated with a serial dilution of compounds (maximum concentration is 100 M, 3-fold serially diluted, 16 points) at room temperature for 1.5 hour in an assay buffer containing 50 mM HEPES, pH 7.0, 100 mM NaCl, 0.01% BSA, 1 mM DTT, 0.01% Triton X-100. Then 8 nM in-house UbcH5B-Ub conjugate protein, 120 nM SRC (aa251-536, in-house), 0.5 mM ATP, 5 mM MgCl₂ and Streptavidin-Tb cryptate (Cat: 610SATLB, Cisbio Bioassays) were added to plate and further incubated at room temperature for 2 hours. The HTRF signals (ex337 nm, em 520 nm/620 nm) were read on BMG PHERAstar FS instrument. The inhibition percentage of Cbl interaction with UbcH5B-Ub in presence of increasing concentrations of compounds was calculated based on the ratio of fluorescence at 520 nm to that at 620 nm. The IC₅₀ for each compound was derived from fitting the data to the four-parameter logistic equation by Dotmatics.

### Biotin-Labelled Cbl Biochemical Assay

Compounds disclosed herein were tested for stabilization of an inactive conformation of Cbl (aa49-434, in-house) and preventing the interaction of Cbl-b with UbcH5B-Ub in an assay based on Homogeneous Time Resolved Fluorescence. 5.0 nM recombinant biotin labelled Cbl was pre-incubated with a serial dilution of compounds (maximum concentration is 100 M, 3-fold serially diluted, 16 points) at room temperature for 1.5 hour in an assay buffer containing 50 mM HEPES, pH 7.0, 100 mM NaCl, 0.01% BSA, 1 mM DTT, 0.01% Triton X-100. Then 10 nM in-house UbcH5B-Ub conjugate protein, 60 nM SRC (aa251-536, in-house), 0.5 mM ATP, 5 mM MgCl₂ and Streptavidin-Tb cryptate (Cat: 610SATLB, Cisbio Bioassays) were added to plate and further incubated at room temperature for 2 hours. The HTRF signals (ex337 nm, em 520 nm/620 nm) were read on BMG PHERAstar FS instrument. The inhibition percentage of Cbl interaction with UbcH5B-Ub in presence of increasing concentrations of compounds was calculated based on the ratio of fluorescence at 520 nm to that at 620 nm. The IC₅₀ for each compound was derived from fitting the data to the four-parameter logistic equation by Dotmatics.

### SPR Assay

### CBLB SPR

Surface plasmon resonance analysis was performed at room temperature using a BIAcore 8K system with SA chips (Cytiva). For all measurements, an HBS-EP buffer (Cytiva) consisting of 10 mM HEPES, 150 mM NaCl, 3 mM EDTA and 0.05% (v/v) surfactant P20 was used as running buffer. CBLB proteins (co-expressed with BirA biotin ligase) were Immobilized to the chip at a concentration of 500 nM. Gradient concentrations of Compound (0, 0.39, 0.78. 1.6, 3.1, 6.3, 13, 25, 50, 100 and 200 nM) were then flowed over the chip surface. After each cycle, the sensor surface was regenerated with 300 mM NaCl. The binding kinetics were all analyzed with the software of BIA evaluation using a 1:1 kinetics mode.

### CBL SPR

Surface plasmon resonance analysis was performed at room temperature using a BIAcore 8K system with SA chips (Cytiva). For all measurements, an HBS-EP buffer (Cytiva) consisting of 10 mM HEPES, 150 mM NaCl, 3 mM EDTA and 0.05% (v/v) surfactant P20 was used as running buffer. CBL proteins (co-expressed with BirA biotin ligase) were Immobilized to the chip at a concentration of 500 nM. Gradient concentrations of Compound (0, 0.39, 0.78. 1.6, 3.1, 6.3, 13, 25, 50, 100 and 200 nM) were then flowed over the chip surface. After each cycle, the sensor surface was regenerated with 300 mM NaCl. The binding kinetics were all analyzed with the software of BIA evaluation using a 1:1 kinetics mode.

The selected compound showed good binding affinity in SPR assay.

### Crystallization Methods

CBLB was incubate with the selected compound for 1 h at 20° C., and the mixture was carried out for initial crystal screening experiments at 293 K by sitting-drop vapor diffusion. The crystals were obtained in the condition consisting of 4% Tacsimate pH6.0, 12% PEG3350 or 0.2M L-Proline, 0.1M hepes pH7.2/pH7.5, 11% PEG3350.

Based on the crystal structure, the ring A of the selected compound of formula (I) forms cation-pi interaction with the guanidine side chain of Cbl-b Arg141. The significant binding energy of this interaction[Kumar K, Woo S M, Siu T, Cortopassi W A, Duarte F, Paton R S. Cation-π interactions in protein-ligand binding: theory and data-mining reveal different roles for lysine and arginine. Chem Sci. 2018 Jan. 31; 9(10):2655-2665] contributes to the high activity of our compound series.

### Cell Assays

### Cellular Auto-Ubiquitylation Assay

HEK293 cells (ATCC), which stably over-expressed Cbl-b or c-Cbl, were maintained in DMEM medium (Thermo Fisher Scientific) supplemented with 10% fetal bovine serum (FBS, Thermo Fisher Scientific), 100 units/mL penicillin and 0.1 mg/mL streptomycin (Thermo Fisher Scientific) in a humidified 37° C. environment with 5% C02.

Cellular auto-ubiquitylation were measured using MSD electrochemiluminescence immunoassays. Streptavidin-coated 384-well MSD plates (Meso Scale Discovery) were coated with 2 g/mL biotinylated Anti-FLAG BioM2 mouse antibody (Sigma) at 2-8° C. overnight. The next day, the plate was washed three times with 1×TBST and blocked with 5% BSA (Sigma) TBST. After 2 h compound treatment, cells were stimulated 10 min with 3 mM PV. Cell lysates were then added to the MSD plate at 25° C. with shaking at 450 rpm for 1 h and then at 2-8° C. overnight to allow binding. Then the lysates were discarded, and plates were washed with 1×TBST three times and incubated with 1 g/mL SULFO-TAG labeled anti-ubiquitinylated protein antibody (Meso Scale Discovery) at 25° C. with shaking at 450 rpm for 2.5 h. Plate was washed again with 1×TBST for three times and subsequently added with MSD GOLD Read Buffer A (Meso Scale Discovery). The signal was recorded on a MESO® SECTOR S600 Reader (Meso Scale Discovery).

The selected compound showed good potency in auto-ubiquitylation assay.

IL-2 Production Assay in Cbl-b or c-Cbl Knockout Jurkat Cells

Jurkat cells (ATCC) were infected with the lentivirus expressing spCas9 and sgRNA targeting human Cbl-b or c-Cbl. Cell clones that stably knockout with Cbl-b or c-Cbl were established and maintained in RPMI 1640 medium (Thermo Fisher Scientific) supplemented with 10% fetal bovine serum (FBS, Thermo Fisher Scientific), 100 units/mL penicillin and 0.1 mg/mL streptomycin (Thermo Fisher Scientific) in a humidified 37° C. environment with 5% C02. Knockout efficiency of Cbl-b or c-Cbl in single cell clones was determined using genomic sequencing and immunoblotting method.

Cbl-b or c-Cbl knockout Jurkat cells were seeded into 96-well plates and then treated with compounds. After 2 h compound treatment, cell suspension in each well was transferred to anti-CD3 (OKT3, Thermo Fisher Scientific) coated high-binding 96-well plates for 24 hours at 37° C. Culture supernatant was collected for subsequent measurement of IL-2 concentration by a TR-FRET-based method as described by the manufacturer manual (Cisbio). FRET signals were measured using a PHERAstar FSX reader (BMG Labtech).

The selected compound showed good potency in IL-2 Production Assay

### Human Cord Blood CD34⁺ Proliferation Assay

Hematopoietic toxicity is one of the major limitations of antitumor agents full-dose using, and it is difficult to generate drugs with stronger antitumor effects and less hematopoietic toxicity (Kurtin S. Myeloid toxicity of cancer treatment. J Adv Pract Oncol. 2012; 3(4):209-24.). In the face of this challenge, in vitro models of hematopoiesis were used increasingly in investigative hematopathology and in preclinical safety studies on candidate drugs. Both colony forming cell assay and proliferation assay of hematopoietic stem cells were successful to predict maximum tolerated dose for compounds (Pessina A, Albella B, Bayo M, Bueren J, Brantom P, Casati S, et al. Application of the CFU-GM assay to predict acute drug-induced neutropenia: an international blind trial to validate a prediction model for the maximum tolerated dose (MTD) of myelosuppressive xenobiotics. Toxicol Sci. 2003; 75(2):355-67, Clarke E, Pereira C, Chaney R, Woodside S, Eaves A C, Damen J. Toxicity testing using hematopoietic stem cell assays. Regen Med. 2007; 2(6):947-56). What's more, we found that some c-Cbl or Cbl-b inhibitors abnormally increased hematopoietic stem cell proliferation. So human cord blood CD34+ cell proliferation assay was established to estimate the potential risk on hematopoietic toxicity of compounds.

Human cord blood CD34⁺ cells (AllCells), were expanded for 7-12 days in IMDM medium (ATCC) supplemented with 10% fetal bovine serum (FBS, Thermo Fisher Scientific), 100 units/mL penicillin, 0.1 mg/mL streptomycin (Thermo Fisher Scientific), 100 ng/mL recombinant human SCF (Peprotech), 50 ng/mL recombinant human TPO (Peprotech), 20 ng/mL recombinant human IL-3 (Peprotech) and 100 ng/mL recombinant human Flt-3L (Peprotech). Cells were cultured in a humidified 37° C. environment with 5% CO₂.

The growth-acceleration activity of compounds in CD34+ cells, was determined using CellTiter-Glo luminescent cell viability assay (Promega). The 5,000 cells were seeded per well of a 96-well in IMDM medium supplemented with 10% fetal bovine serum, 100 units/mL penicillin, 0.1 mg/mL streptomycin and 10 ng/mL recombinant human SCF. Following a 7-day exposure to compounds disclosed herein, CellTiter-Glo reagent was added in each well. Mixture was mixed on an orbital shaker for 2 minutes to allow cell lysis, followed by 10 minutes incubation at room temperature to allow stabilization of luminescent signal, Luminescent signal was measured using a PHERAstar FSX reader (BMG Labtech). The selected compounds showed low or moderate potency in Human cord blood CD34+ proliferation assay

### In Vitro Plasma Stability

### Materials

| Reagent | Vendor | Cat No. | Lot No. |
|---|---|---|---|
| Human plasma | — | — | 220517-3/-4 |
| Dog plasma | Marshall | — | BW9768244 |
| | Biotechnology | | |
| SD Rat plasma | Beigene-in house | — | — |
| Mouse plasma | Charles river | Mice plasma- | VR-MICE-PL- |
| | | ICR-M-5Ml | EK2-20200914 |
| Benfluorex | Sigma | B7522-5G | 087F0342 |
| Bisacodyl | TIC | B5066 | YSR4M-QC |

### Procedure

The frozen plasma is thawed at 4° C. and then centrifuge for 10 min,

Dissolve the test compound(s) and positive control with DMSO to 100× the final concentration at 0.1 mM, 2 μL of 0.1 mM control and test compounds were add to the 198 μL plasma, Mix the sample well by briefly vortexing and then incubate at 37° C. in a water bath and shake gently. The experiment will be conducted in duplicate. The final organic solvent concentration in the assay should always be ≤10%. An aliquot of 20 uL of samples was taken from the plasma to 200 uL cold ACN (containing IS) at 0 h, 0.5 h, 2 h, and 4 h. The mixture was vortexed for 1 min and centrifuged at 4000 rpm for 10 min. An aliquot of 30 μL supernatant was diluted with 120 μL ACN/H₂O, and then injected for LC-MS/MS analysis.

### Data Analysis

All calculations were carried out using Microsoft Excel. Peak areas were determined form extracted ion chromatograms. B Remaining was calculated using Peak areas.

The selected compounds were stable in rat plasma.

### Activity Tables

Each of the compounds in Tables 1, 2 and 3 was tested in one or more of the Cbl-b biochemical assays and was found to have activity therein.

TABLE 1

| | Biotin-labelled | Biotin-labelled | co-express | co-express |
|---|---|---|---|---|
| Compound | CBLB/E2-Ub- | CBL/E2-Ub- | CBLB/E2-Ub- | CBL/E2-Ub- |
| number (Series | binding assay | binding assay | binding assay | binding assay IC50 |
| A) | IC50 (nM) | IC50 (nM) | IC50 (nM) | (nM) |
| A1 | 5.36 | 31.8 | | |
| A2 | 3.26 | 23 | | |
| A3 | 5.8 | 60.1 | 7.1 | 47 |
| A4 | 3.3 | 28.7 | | |
| A5 | 138.0 | 914.0 | | |
| A6 | | | 7.2 | 19.8 |
| A7 | | | 4.4 | 14.4 |
| A8 | | | 6.0 | 18.7 |
| A9 | | | 12.8 | 24.3 |
| A10 | | | 12.0 | 70.1 |
| A11 | | | 6.6 | 46.2 |
| A12 | | | 3.9 | 32.4 |
| A13 | | | 6.1 | 47.0 |
| A14 | | | 45.3 | 185.0 |
| A15 | | | 0.9 | 4.0 |
| A16 | | | 0.9 | 5.9 |
| A17 | | | 0.8 | 20.7 |
| A18 | | | 1.1 | 6.0 |
| A19 | | | 1.7 | 8.2 |
| A20 | | | 3.0 | 26.5 |
| A21 | | | 2.9 | 31.0 |
| A22 | | | 1.1 | 3.6 |
| A23 | | | 1.4 | 11.4 |
| A24 | | | 2.9 | 8.6 |
| A25 | | | 1.5 | 10.5 |
| A26 | | | 1.3 | 6.3 |
| A27 | | | 1.4 | 8.0 |
| A28 | | | 1.0 | 2.4 |
| A29 | | | 1.3 | 2.3 |
| A30 | | | 0.6 | 6.4 |
| A31 | | | 0.7 | 1.3 |
| A32 | | | 1.1 | 4.9 |
| A33 | | | 1.5 | 1.1 |
| A34 | | | 2.6 | 7.8 |
| A35 | | | 2.2 | 13.0 |
| A36 | | | 1.7 | 16.0 |
| A37 | | | 0.8 | 4.8 |
| A38 | | | 1.4 | 2.4 |
| A39 | | | 2.7 | 14.1 |
| A40 | | | 1.6 | 3.1 |
| A41 | | | 0.2 | 0.9 |
| A42 | | | 1.8 | 7.6 |
| A43 | | | 2.0 | 12.0 |
| A44 | | | 1.8 | 7.7 |
| A45 | | | 1.2 | 3.5 |
| A46 | | | 1.6 | 5.9 |
| A47 | | | 1.4 | 7.0 |
| A48 | | | 0.8 | 2.7 |
| A49 | | | 3.0 | 11.9 |
| A50 | | | 0.9 | 1.1 |
| A51 | | | 1.5 | 4.3 |
| A52 | | | 0.8 | 3.4 |
| A53 | | | 2.0 | 7.7 |
| A54 | | | 1.9 | 6.0 |
| A55 | | | 2.9 | 6.7 |
| A56 | | | 1.5 | 4.1 |
| A57 | | | 0.6 | 1.8 |
| A58 | | | 0.7 | 2.6 |
| A59 | | | 1.7 | 5.3 |
| A60 | | | 0.5 | 1.1 |
| A61 | | | 1.2 | 11.1 |
| A62 | | | 1.1 | 7.0 |
| A63 | | | 1.1 | 1.8 |
| A64 | | | 2.2 | 26.0 |
| A65 | | | 0.9 | 2.0 |
| A66 | | | 1.1 | 3.4 |
| A67 | | | 0.5 | 2.8 |
| A68 | | | 0.9 | 2.9 |
| A69 | | | 0.8 | 1.7 |
| A70 | | | 1.1 | 4.1 |
| A71 | | | 0.6 | 0.9 |
| A72 | | | 0.4 | 0.6 |
| A73 | | | 1.2 | 2.8 |
| A74 | | | 1.2 | 4.2 |
| A75 | | | 1.7 | 5.5 |
| A76 | | | 2.6 | 8.3 |
| A77 | | | 0.40 | 1.2 |
| A78 | | | 1.2 | 2.3 |
| A79 | | | 0.73 | 1.4 |
| A80 | | | 2.0 | 13 |
| A81 | | | 1.8 | 8.2 |
| A82 | | | 0.70 | 1.8 |
| A83 | | | 1.0 | 1.7 |
| A84 | | | 0.5 | 12.0 |
| A85 | | | 3.4 | 26.0 |
| A86 | | | 2.7 | 13.0 |
| A87 | | | 1.5 | 12.0 |
| A88 | | | 4.4 | 20.0 |
| A89 | | | 1.7 | 45.0 |
| A90 | | | 0.5 | 3.2 |
| A91 | | | 0.6 | 4.1 |
| A92 | | | 0.4 | 2.5 |
| A93 | | | 1.6 | 5.2 |
| A94 | | | 0.5 | 5.9 |
| A95 | | | 0.7 | 3.4 |
| A96 | | | 0.5 | 3.2 |

TABLE 2

| | co-express | co-express |
|---|---|---|
| Compound | CBLB/E2-Ub- | CBL/E2-Ub- |
| number (Series | binding assay | binding assay IC50 |
| B) | IC50 (nM) | (nM) |
| B1 | 0.7 | 2.1 |
| B2 | 1.8 | 4.7 |
| B3 | 0.7 | 1.8 |
| B4 | 3.0 | 12.0 |
| B5 | 1.6 | 16.0 |
| B6 | 4.0 | 6.9 |
| B7 | 1.9 | 6.4 |
| B8 | 1.8 | 21.0 |
| B9 | 25.9 | 42.9 |
| B10 | 2.0 | 15.6 |
| B11 | 1.3 | 14.2 |
| B12 | 23.9 | 51.6 |
| B13 | 30.5 | 89.1 |
| B14 | 13.6 | 89.9 |
| B15 | 0.9 | 1.9 |
| B16 | 1.7 | 16.1 |
| B17 | 1.22 | 3.84 |
| B18 | 1.6 | 13.0 |
| B19 | 10.3 | 77.7 |
| B20 | 1.8 | 3.9 |
| B21 | 2.2 | 7.5 |
| B22 | 4.0 | 19.5 |
| B23 | 3.6 | 8.8 |
| B24 | 0.9 | 17.0 |
| B25 | 1.2 | 1.1 |
| B26 | 1.3 | 4.1 |
| B27 | 1.2 | 8.4 |
| B28 | 0.8 | 2.2 |
| B29 | 1.2 | 2.3 |
| B30 | 1.4 | 8.8 |
| B31 | 0.8 | 0.7 |
| B32 | 1.0 | 1.5 |
| B33 | 0.9 | 4.7 |
| B34 | 1.6 | 5.5 |
| B35 | 0.7 | 1.1 |
| B36 | 0.8 | 2.3 |
| B37 | 0.8 | 4.5 |
| B38 | 0.5 | 1.3 |
| B39 | 0.8 | 8.8 |
| B40 | 1.0 | 2.1 |
| B41 | 0.8 | 2.3 |
| B42 | 2.1 | 4.7 |
| B43 | 0.6 | 0.6 |
| B44 | 1.9 | 9.0 |
| B45 | 1.7 | 16.5 |
| B46 | 0.7 | 2.7 |
| B47 | 2.2 | 6.1 |
| B48 | 0.89 | 3.2 |
| B49 | 3.2 | 14 |
| B50 | 1.5 | 9.3 |
| B51 | 1.4 | 13 |
| B52 | 5.0 | 19.0 |
| B53 | 3.1 | 9.7 |
| B54 | 1.2 | 12.0 |
| B55 | 1.6 | 14.0 |
| B56 | 1.4 | 32.0 |
| B57 | 1.3 | 15.0 |

TABLE 3

| | Biotin-labelled | Biotin-labelled | co-express | co-express |
|---|---|---|---|---|
| Compound | CBLB/E2-Ub- | CBL/E2-Ub- | CBLB/E2-Ub- | CBL/E2-Ub- |
| number (Series | binding assay | binding assay | binding assay | binding assay IC50 |
| C) | IC50 (nM) | IC50 (nM) | IC50 (nM) | (nM) |
| C1 | 11 | 60 | | |
| C2 | 11.9 | 28.8 | | |
| C3 | 4.8 | 21.7 | | |
| C4 | 16.2 | 76.0 | | |
| C5 | 8.3 | 29.1 | | |
| C6 | 3.4 | 48.0 | | |
| C7 | | | 2.56 | 19.7 |
| C8 | | | 1.0 | 1.8 |
| C9 | | | 0.9 | 15.7 |
| C10 | | | 2.3 | 26.6 |
| C11 | | | 0.8 | 1.5 |
| C12 | | | 1.4 | 1.3 |
| C13 | | | 1.1 | 9.2 |
| C14 | | | 2.0 | 6.6 |
| C15 | | | 1.8 | 5.2 |
| C16 | | | 1.7 | 5.3 |
| C17 | | | 1.5 | 1.1 |
| C18 | | | 1.4 | 3.1 |
| C19 | | | 2.7 | 6.0 |
| C20 | | | 6.9 | 79.0 |
| C21 | | | 1.8 | 17.7 |
| C22 | | | 1.4 | 6.1 |
| C23 | | | 6.3 | 100.0 |
| C24 | | | 1.6 | 10.4 |
| C25 | | | 2.8 | 49.5 |
| C26 | | | 2.3 | 25.0 |
| C27 | | | 1.0 | 3.5 |
| C28 | | | 0.8 | 3.3 |
| C29 | | | 4.2 | 9.6 |
| C30 | | | 1.4 | 3.3 |
| C31 | | | 3.9 | 17.0 |
| C32 | | | 1.87 | 7.72 |
| C33 | | | 11.4 | 46.7 |
| C34 | | | 1.3 | 5.1 |
| C35 | | | 3.8 | 24.4 |
| C36 | | | 1.8 | 2.8 |
| C37 | | | 0.9 | 2.4 |
| C38 | | | 1.1 | 1.5 |
| C39 | | | 0.8 | 2.9 |
| C40 | | | 2.5 | 11.5 |
| C41 | | | 1.5 | 7.0 |
| C42 | | | 1.5 | 25.1 |
| C43 | | | 1.3 | 10.1 |
| C44 | | | 1.5 | 3.3 |
| C45 | | | 1.3 | 5.4 |
| C46 | | | 0.7 | 1.8 |
| C47 | | | 0.7 | 1.2 |
| C48 | | | 2.7 | 6.3 |
| C49 | | | 2.0 | 5.9 |
| C50 | | | 0.7 | 0.8 |
| C51 | | | 1.5 | 4.9 |
| C52 | | | 3.3 | 14.8 |
| C53 | | | 3.7 | 15.8 |
| C54 | | | 1.3 | 6.8 |
| C55 | | | 1.1 | 1.9 |
| C56 | | | 0.5 | 2.6 |
| C57 | | | 0.9 | 3.4 |
| C58 | | | 1.7 | 3.2 |
| C59 | | | 2.5 | 7.3 |
| C60 | | | 1.1 | 9.2 |

A number of references have been cited, the disclosures of which are incorporated herein by reference in their entirety.

What is claimed is:

1. A compound of formula (I): 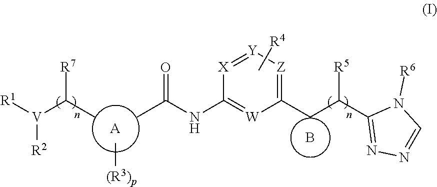
or a pharmaceutically acceptable salt, tautomer, stereoisomer, isotopologue, or enantiomer thereof,
wherein:
each of V, W, X, Y, and Z is, independently CH or N;
each of R¹ and R² is, independently, hydrogen, substituted or unsubstituted C₁₋₈ alkyl, substituted or unsubstituted cycloalkyl, substituted or unsubstituted non-aromatic heterocyclyl, substituted or unsubstituted saturated cycloalkylalkyl, substituted or unsubstituted non-aromatic heterocyclylalkyl, or R¹ and R² together with the atom which R¹ and R² connect to form a substituted or unsubstituted cycloalkyl or non-aromatic heterocyclyl;
R³ is hydrogen, halogen, —CN, hydroxyl, substituted or unsubstituted C₁₋₈ alkyl, or substituted or unsubstituted saturated cycloalkyl;
R⁴ is hydrogen, halogen, substituted or unsubstituted C₁₋₈ alkyl, or substituted or unsubstituted saturated cycloalkyl;
R⁵ is hydrogen, halogen, substituted or unsubstituted C₁₋₈ alkyl, or substituted or unsubstituted saturated cycloalkyl;
R⁶ is hydrogen, or substituted or unsubstituted C₁₋₈ alkyl;
R⁷ is hydrogen, deuterium, halogen, or substituted or unsubstituted C₁₋₈ alkyl or substituted or unsubstituted saturated cycloalkyl;
ring A is substituted or unsubstituted aryl, or substituted or unsubstituted heteroaryl;
moiety B is substituted or unsubstituted aryl, substituted or unsubstituted cycloalkyl, substituted or unsubstituted cycloalkylalkyl, a substituted or unsubstituted saturated spiro bicyclic ring, or substituted or unsubstituted non-aromatic heterocyclyl; and
each of m, n and p is, independently, 0, 1, or 2.

2-6. (canceled)

7. The compound of claim 1, wherein R¹ is 2-methylpropyl, neopentyl (R)-(sec-butyl), 1-methylcyclopropyl 1-methylcyclobutyl, (1S,3S)-3-methylcyclobutyl, 3-methylcyclobutyl, (R)-3,3-difluorocyclopentyl, cyclopropylmethyl, (S)-3,3-difluorocyclopentyl, (S)-1-cyclopropylethyl, (1-methylcyclopropyl)methyl, (1-fluorocyclopropyl)methyl, cyclobutylmethyl, 1-cyclobutylethyl, (S)-1-cyclobutylethyl, (1-methylcyclobutyl)methyl, (1-fluorocyclobutyl)methyl, cyclopentylmethyl, (1-methylcyclopentyl)methyl, (1-fluorocyclopentyl)methyl, 1-cyclopentylethyl, (S)-1-cyclopentylethyl, (1-methylcyclopentyl)methyl, cyclohexylmethyl, 1-cyclohexylethyl, (1-methylcyclohexyl)methyl, (4,4-difluorocyclohexyl)methyl, (3,3-difluorocyclohexyl)methyl, (3,3-difluorocyclopentyl)methyl, cyclohexyl, or cycloheptyl.

8. The compound of claim 1, wherein R² is hydrogen.

9. The compound of claim 1, wherein the compound is a compound of formula (IIIa): 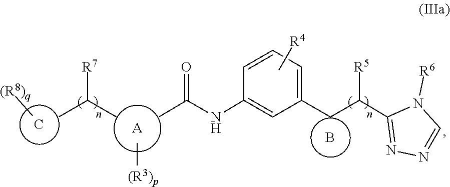
wherein moiety C is a substituted or unsubstituted saturated spiro bicyclic ring, substituted or unsubstituted non-aromatic heterocyclyl, or substituted or unsubstituted amino;
R⁸ is hydrogen, halogen, substituted or unsubstituted C₁₋₈ alkyl, substituted or unsubstituted C₁₋₈ alkoxyl, substituted or unsubstituted saturated cycloalkyl, or substituted or unsubstituted saturated cycloalkylalkyl; or two R⁸ together with the atom(s) which they connect to form a substituted or unsubstituted saturated cycloalkyl or substituted or unsubstituted saturated spiro cyclic ring; and
q is 0, 1, 2, or 3.

10. The compound of claim 1, wherein the compound is a compound of formula (IIIb): 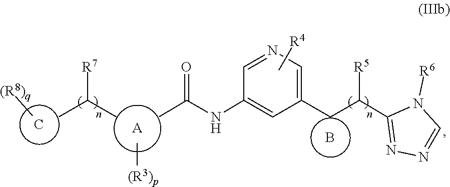
wherein moiety C is a substituted or unsubstituted saturated spiro bicyclic ring, substituted or unsubstituted non-aromatic heterocyclyl, or substituted or unsubstituted amino;
R⁸ is hydrogen, halogen, substituted or unsubstituted C₁₋₈ alkyl, substituted or unsubstituted C₁₋₈ alkoxyl, substituted or unsubstituted saturated cycloalkyl, or substituted or unsubstituted saturated cycloalkylalkyl; or two R⁸ together with the atom(s) which they connect to form a substituted or unsubstituted saturated cycloalkyl or substituted or unsubstituted saturated spiro cyclic ring; and
q is 0, 1, 2, or 3.

11. (canceled)

12. The compound of claim 1, wherein ring A is

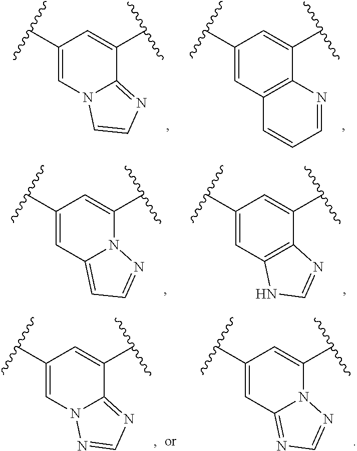 [SMILES: *c1cc(*)c2nccn2c1 ; *c1cc(*)c2ncccc2c1 ; *c1cc(*)n2nccc2c1 ; *c1cc(*)c2nc[nH]c2c1 ; *c1cc(*)c2ncnn2c1 ; *c1cc(*)n2ncnc2c1]

13. (canceled)

14. The compound of claim 12, wherein R³ is hydrogen, F, Cl, or methyl.

15. The compound of claim 12, wherein

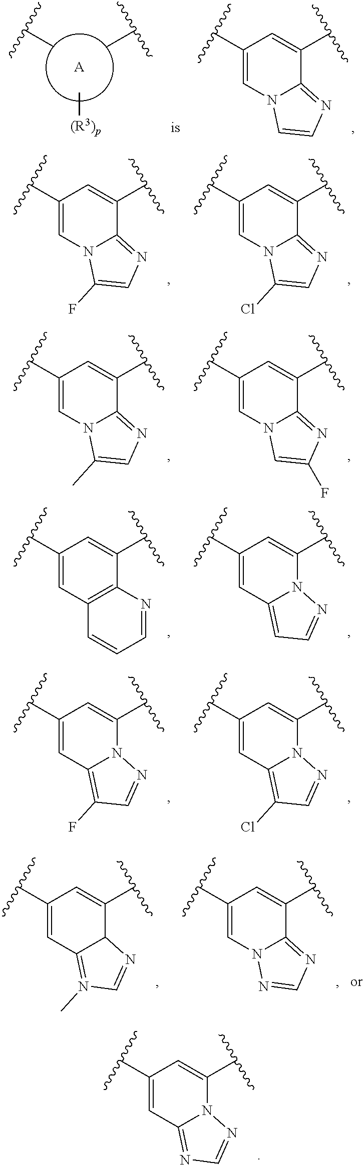

16. (canceled)

17. The compound of claim 12, wherein moiety B is

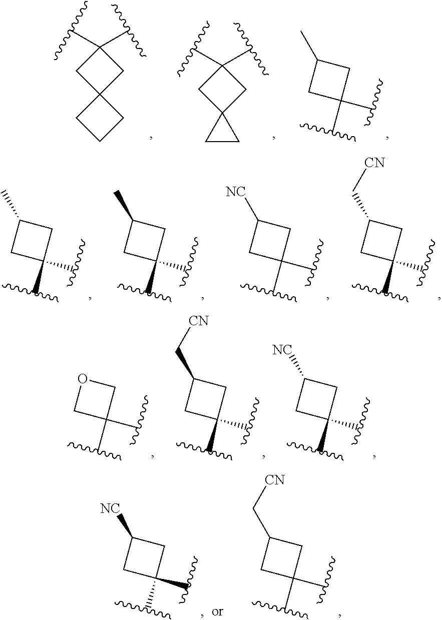

18-20. (canceled)

21. The compound of claim 12, wherein

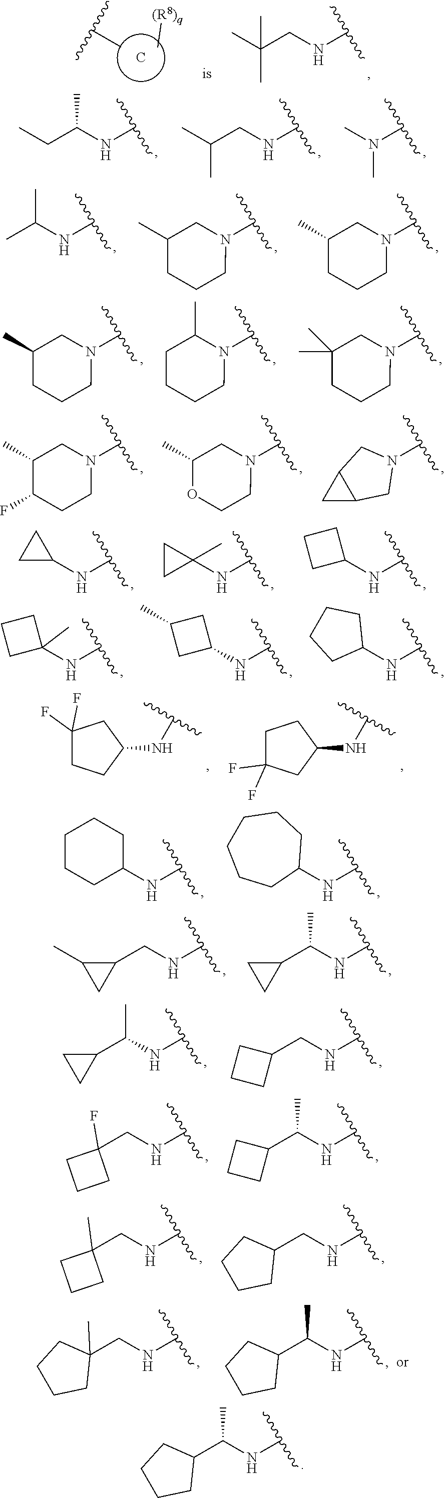

22. The compound of claim 1, wherein ring A is

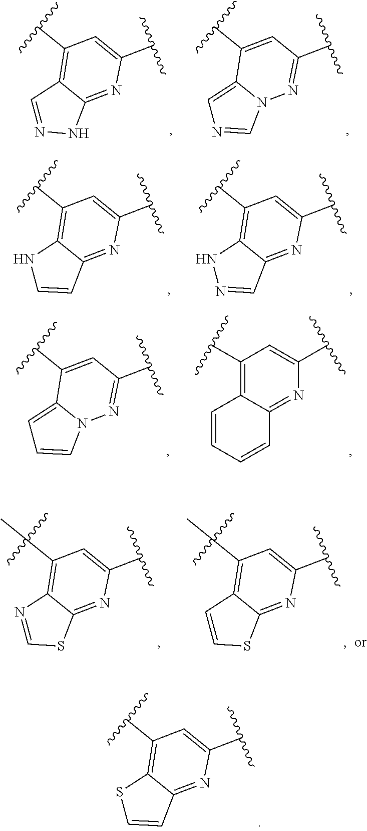

23-24. (canceled)

25. The compound of claim 22, wherein R³ is H, F, Cl, Br or methyl.

26. The compound of claim 22, wherein

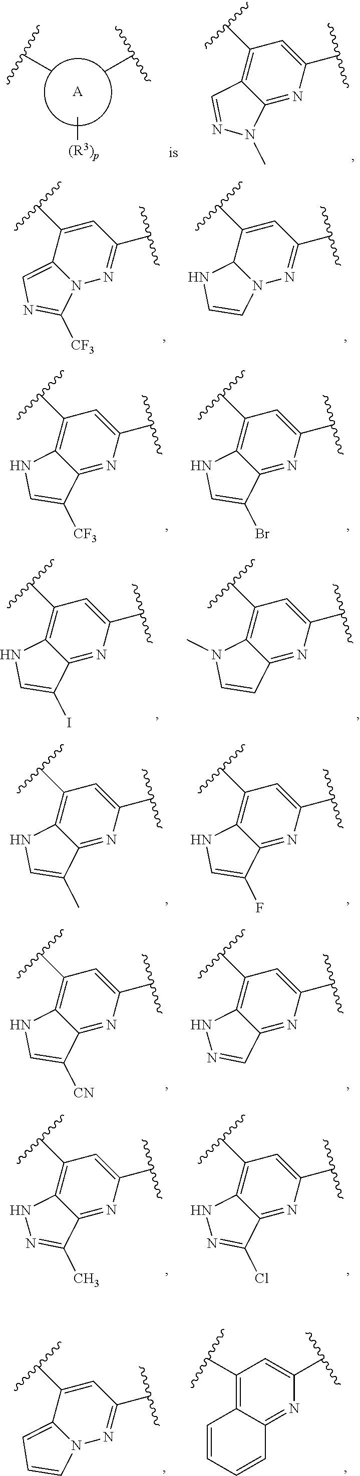

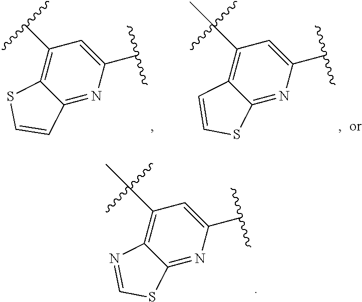 [SMILES: *c1cc(*)c2sccc2n1 ; *c1cc(*)c2ccsc2n1 ; *c1cc(*)c2ncsc2n1]

27-28. (canceled)

29. The compound of claim 22, wherein moiety B is

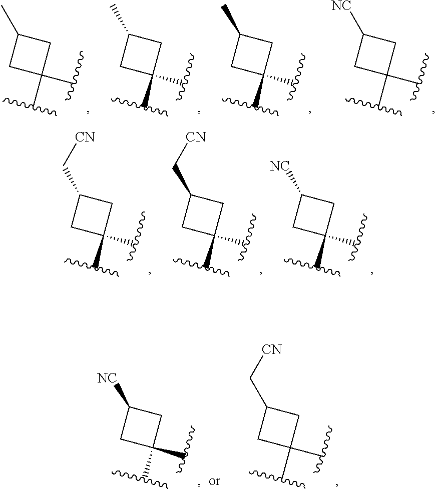

30-32. (canceled)

33. The compound of claim 22, wherein

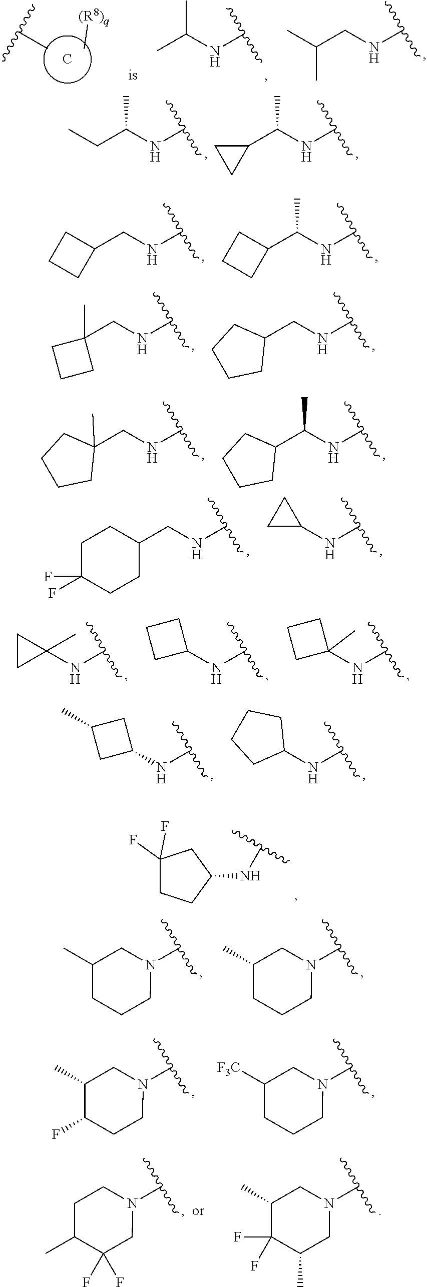

34. The compound of claim 1, wherein ring A is

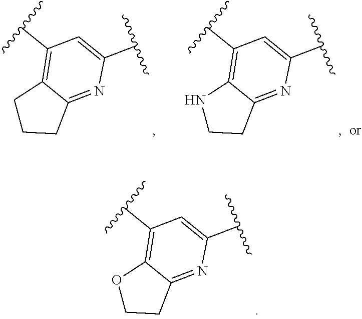 [SMILES: *c1cc(*)c2c(n1)CCC2 ; *c1cc(*)c2c(n1)CCN2 ; *c1cc(*)c2c(n1)CCO2]

35. The compound of claim 34, wherein R³ is hydrogen, halogen, hydroxyl, or substituted or unsubstituted C₁₋₄ alkyl.

36. The compound of claim 35, wherein p is 2.

37. The compound of claim 34, wherein

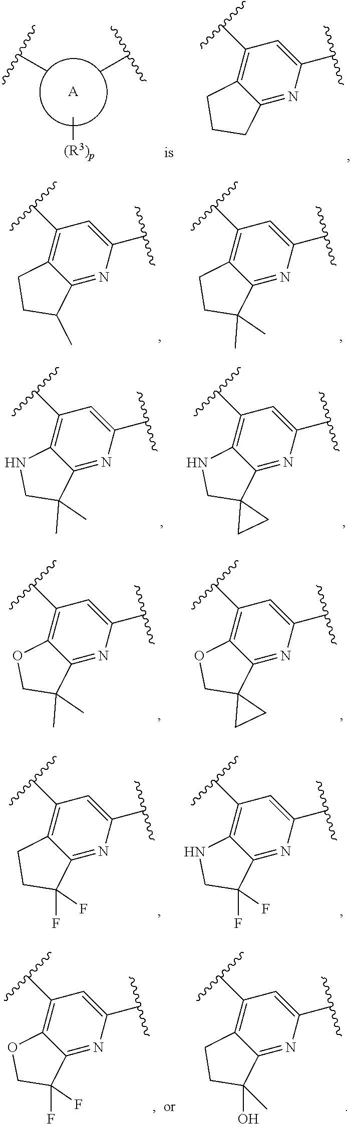

38. (canceled)

39. The compound of claim 34, wherein moiety B is

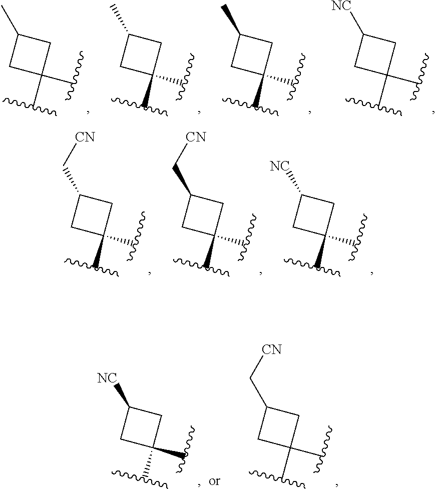

40-41. (canceled)

42. The compound of claim 34, wherein

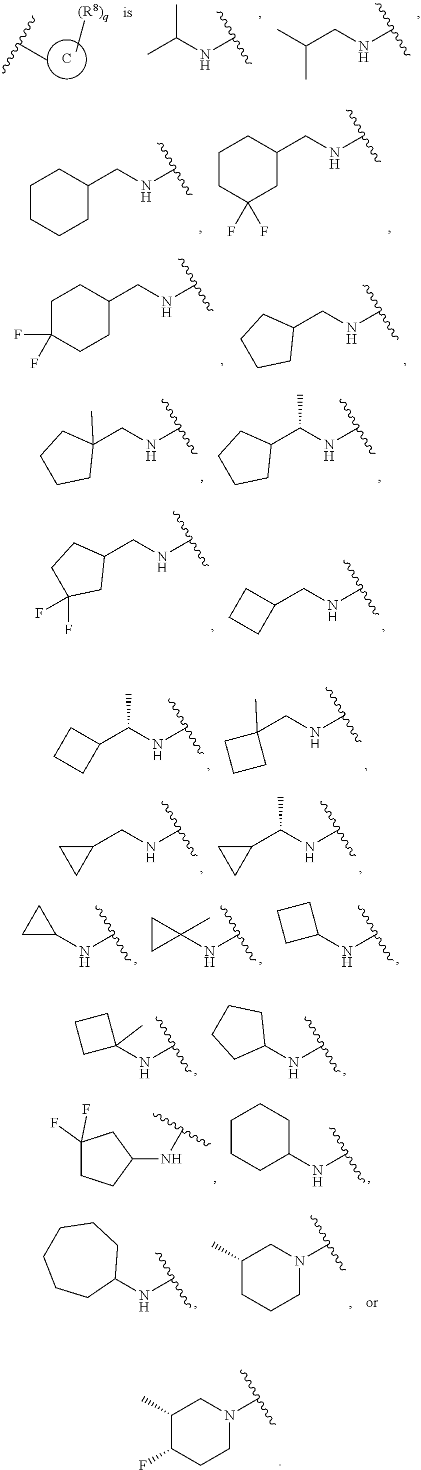

43. The compound of claim 1, wherein R⁵ is hydrogen, or methyl.

44. The compound of claim 1, wherein R⁶ is methyl, methyl-d3, or ethyl.

45. (canceled)

46. The compound of claim 1, wherein each of n and p is, independently, 0 or 1, and m is 1.

47-48. (canceled)

49. The compound of claim 1, wherein R⁷ is hydrogen, deuterium, methyl, ethyl, isopropyl or 2-hydroxyethyl.

50. The compound of claim 1, wherein the compound is selected from: 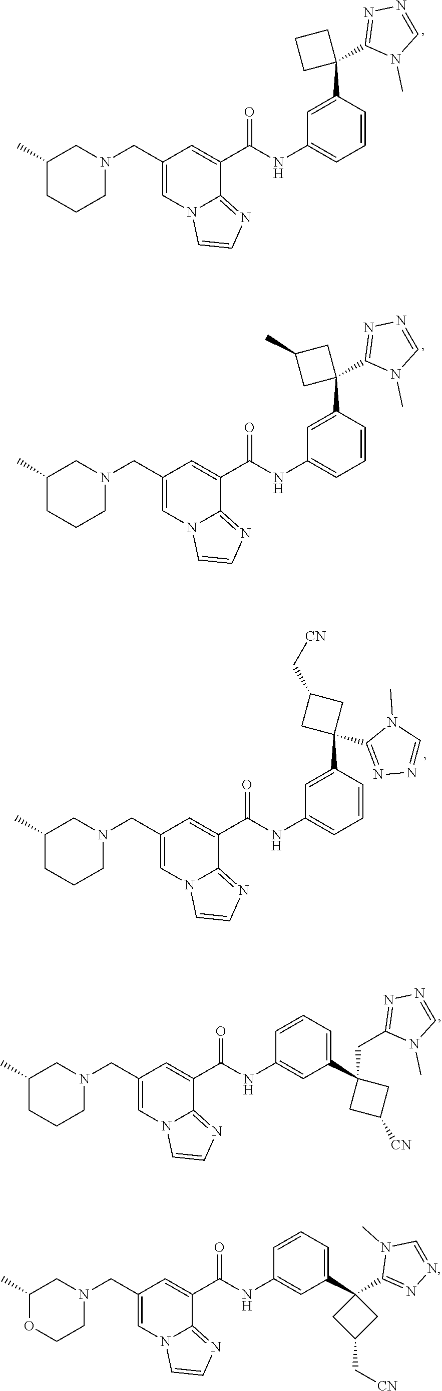 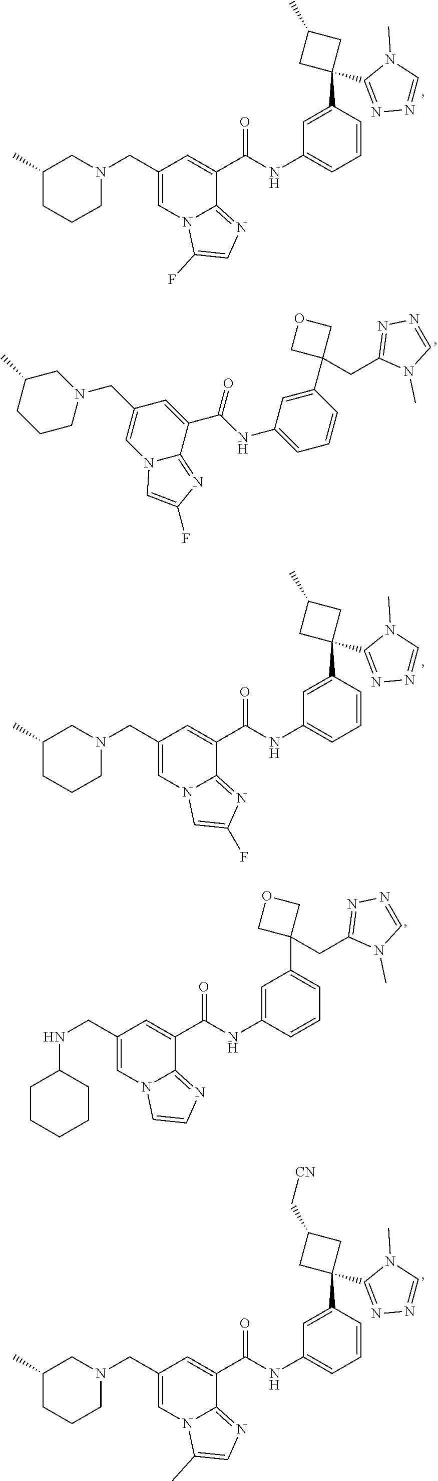 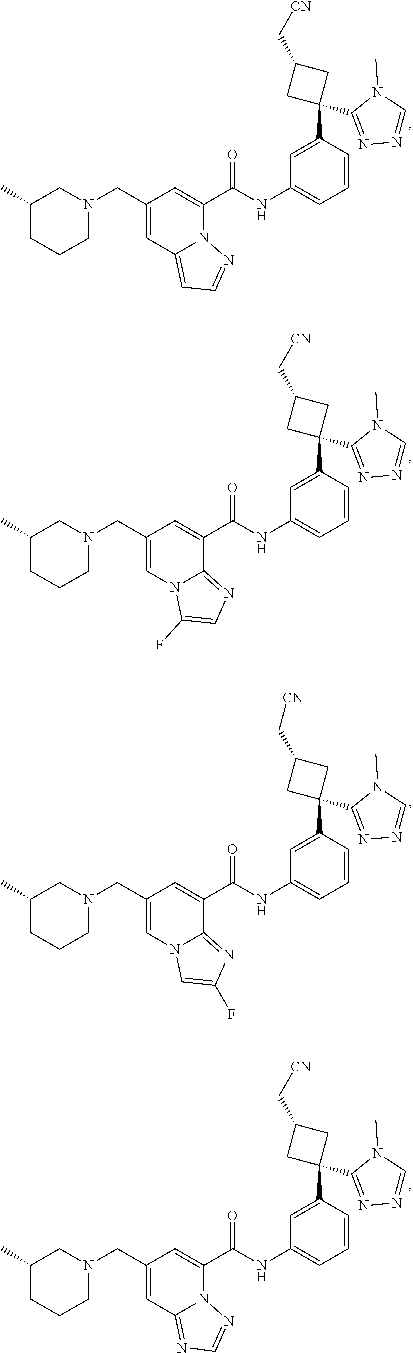 [SMILES: C[C@H]1CCCN(Cc2cc(C(=O)Nc3cccc([C@]4(c5nncn5C)C[C@@H](CC#N)C4)c3)n3nccc3c2)C1 ; C[C@H]1CCCN(Cc2cc(C(=O)Nc3cccc([C@]4(c5nncn5C)C[C@@H](CC#N)C4)c3)c3ncc(F)n3c2)C1 ; C[C@H]1CCCN(Cc2cc(C(=O)Nc3cccc([C@]4(c5nncn5C)C[C@@H](CC#N)C4)c3)c3nc(F)cn3c2)C1 ; C[C@H]1CCCN(Cc2cc(C(=O)Nc3cccc([C@]4(c5nncn5C)C[C@@H](CC#N)C4)c3)n3ncnc3c2)C1] 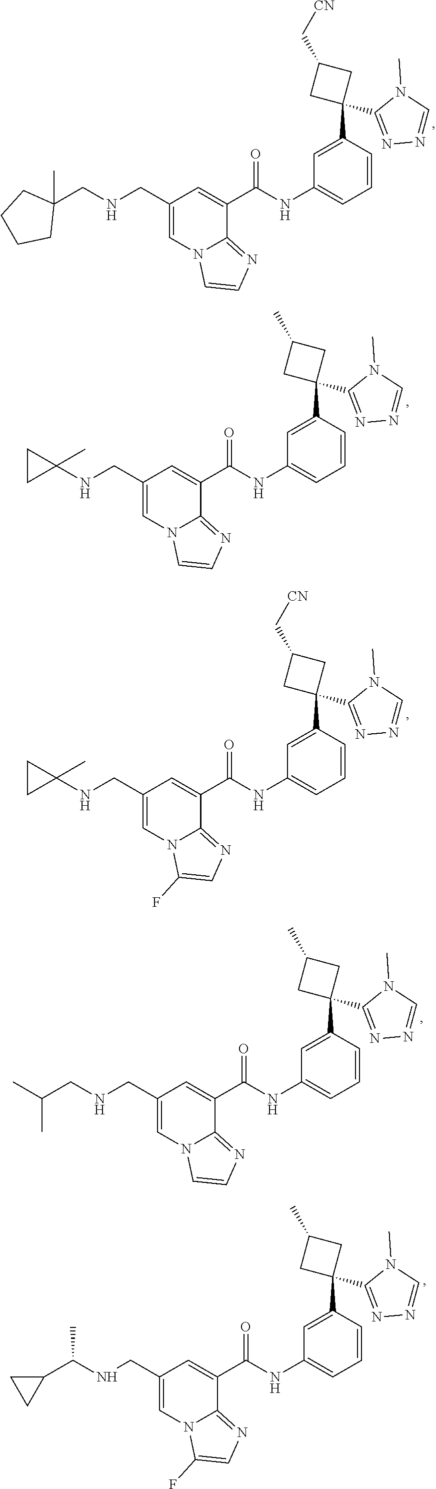 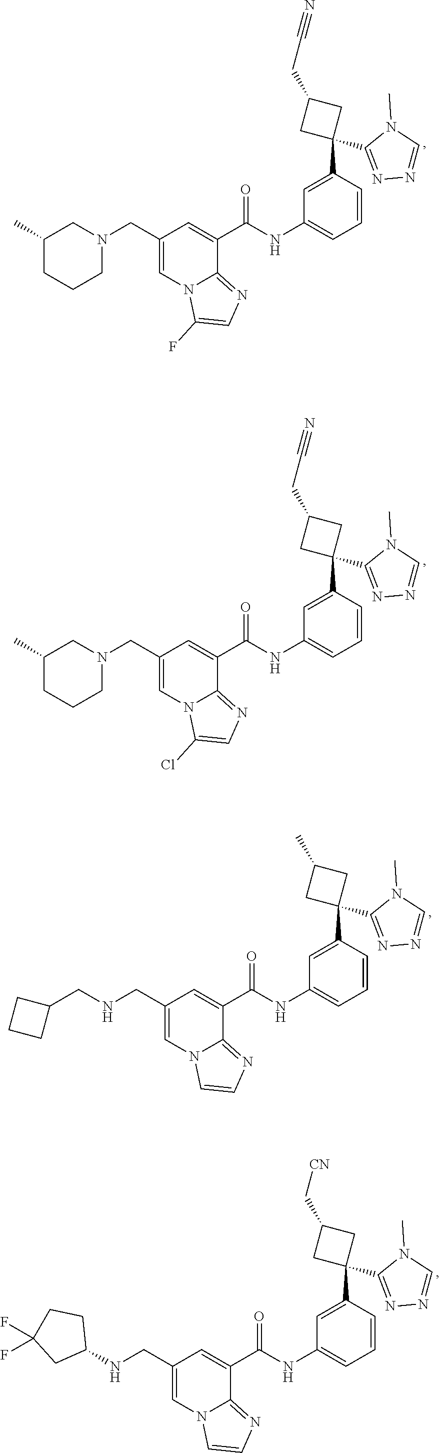 [SMILES: C[C@H]1CCCN(Cc2cc(C(=O)Nc3cccc([C@]4(c5nncn5C)C[C@@H](CC#N)C4)c3)c3ncc(F)n3c2)C1 ; C[C@H]1CCCN(Cc2cc(C(=O)Nc3cccc([C@]4(c5nncn5C)C[C@@H](CC#N)C4)c3)c3ncc(Cl)n3c2)C1 ; Cn1cnnc1[C@]1(c2cccc(NC(=O)c3cc(CNCC4CCC4)cn4ccnc34)c2)C[C@H](C)C1 ; Cn1cnnc1[C@]1(c2cccc(NC(=O)c3cc(CN[C@H]4CCC(F)(F)C4)cn4ccnc34)c2)C[C@H](CC#N)C1] 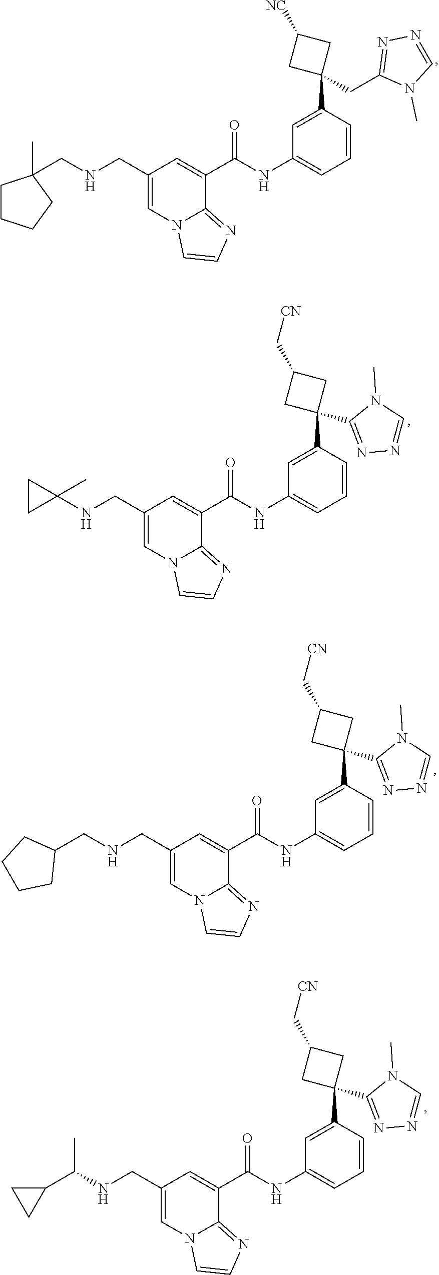 [SMILES: Cn1cnnc1C[C@]1(c2cccc(NC(=O)c3cc(CNCC4(C)CCCC4)cn4ccnc34)c2)C[C@H](C#N)C1 ; Cn1cnnc1[C@]1(c2cccc(NC(=O)c3cc(CNC4(C)CC4)cn4ccnc34)c2)C[C@H](CC#N)C1 ; Cn1cnnc1[C@]1(c2cccc(NC(=O)c3cc(CNCC4CCCC4)cn4ccnc34)c2)C[C@H](CC#N)C1 ; C[C@H](NCc1cc(C(=O)Nc2cccc([C@]3(c4nncn4C)C[C@@H](CC#N)C3)c2)c2nccn2c1)C1CC1] 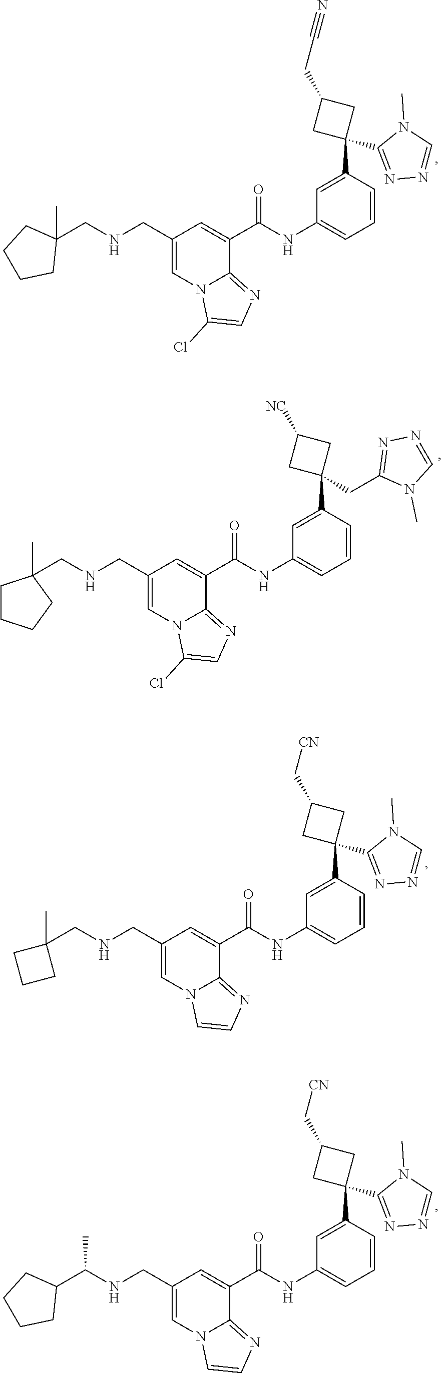 [SMILES: Cn1cnnc1[C@]1(c2cccc(NC(=O)c3cc(CNCC4(C)CCCC4)cn4c(Cl)cnc34)c2)C[C@H](CC#N)C1 ; Cn1cnnc1C[C@]1(c2cccc(NC(=O)c3cc(CNCC4(C)CCCC4)cn4c(Cl)cnc34)c2)C[C@H](C#N)C1 ; Cn1cnnc1[C@]1(c2cccc(NC(=O)c3cc(CNCC4(C)CCC4)cn4ccnc34)c2)C[C@H](CC#N)C1 ; C[C@H](NCc1cc(C(=O)Nc2cccc([C@]3(c4nncn4C)C[C@@H](CC#N)C3)c2)c2nccn2c1)C1CCCC1] 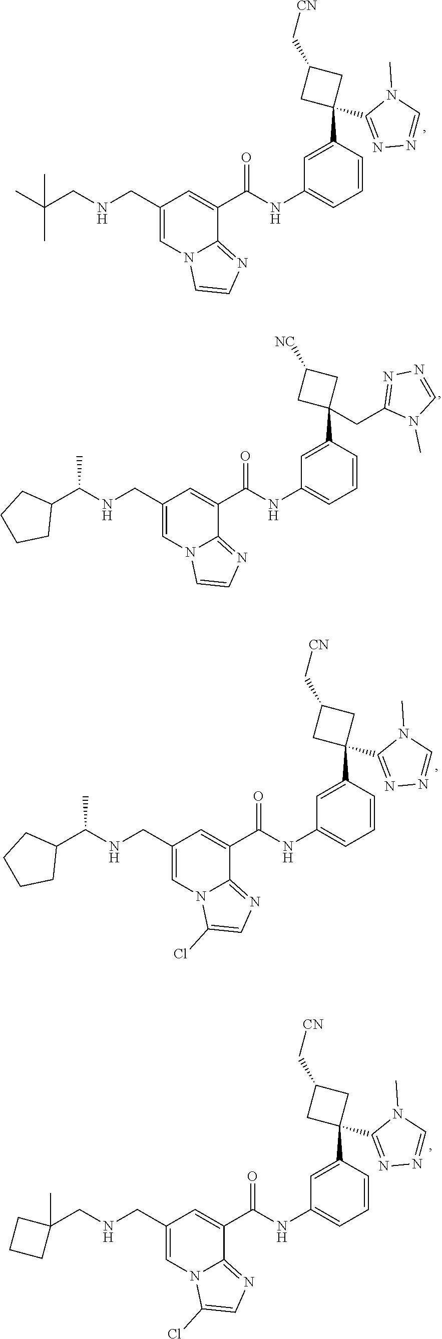 [SMILES: Cn1cnnc1[C@]1(c2cccc(NC(=O)c3cc(CNCC(C)(C)C)cn4ccnc34)c2)C[C@H](CC#N)C1 ; C[C@H](NCc1cc(C(=O)Nc2cccc([C@]3(Cc4nncn4C)C[C@@H](C#N)C3)c2)c2nccn2c1)C1CCCC1 ; C[C@H](NCc1cc(C(=O)Nc2cccc([C@]3(c4nncn4C)C[C@@H](CC#N)C3)c2)c2ncc(Cl)n2c1)C1CCCC1 ; Cn1cnnc1[C@]1(c2cccc(NC(=O)c3cc(CNCC4(C)CCC4)cn4c(Cl)cnc34)c2)C[C@H](CC#N)C1] 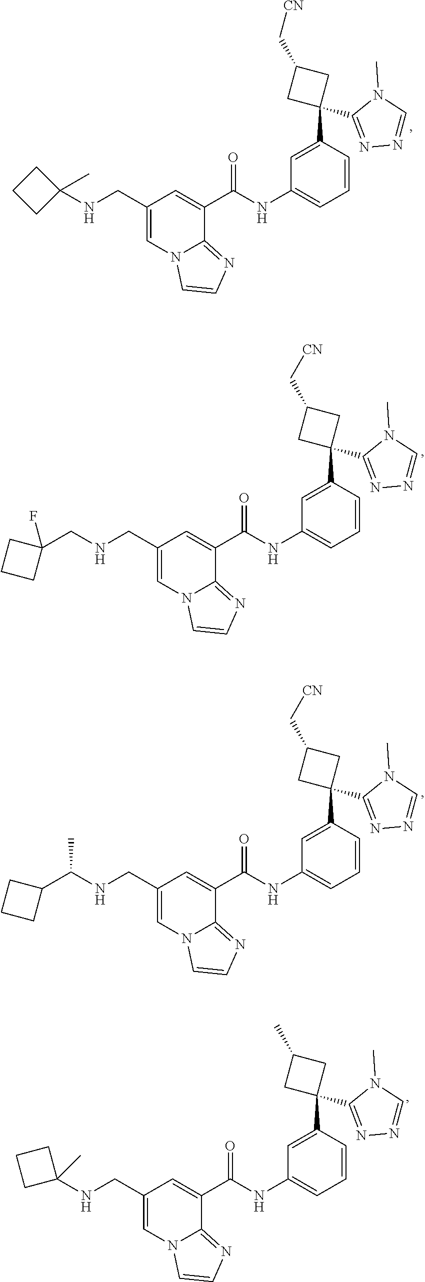 [SMILES: Cn1cnnc1[C@]1(c2cccc(NC(=O)c3cc(CNC4(C)CCC4)cn4ccnc34)c2)C[C@H](CC#N)C1 ; Cn1cnnc1[C@]1(c2cccc(NC(=O)c3cc(CNCC4(F)CCC4)cn4ccnc34)c2)C[C@H](CC#N)C1 ; C[C@H](NCc1cc(C(=O)Nc2cccc([C@]3(c4nncn4C)C[C@@H](CC#N)C3)c2)c2nccn2c1)C1CCC1 ; Cn1cnnc1[C@]1(c2cccc(NC(=O)c3cc(CNC4(C)CCC4)cn4ccnc34)c2)C[C@H](C)C1] 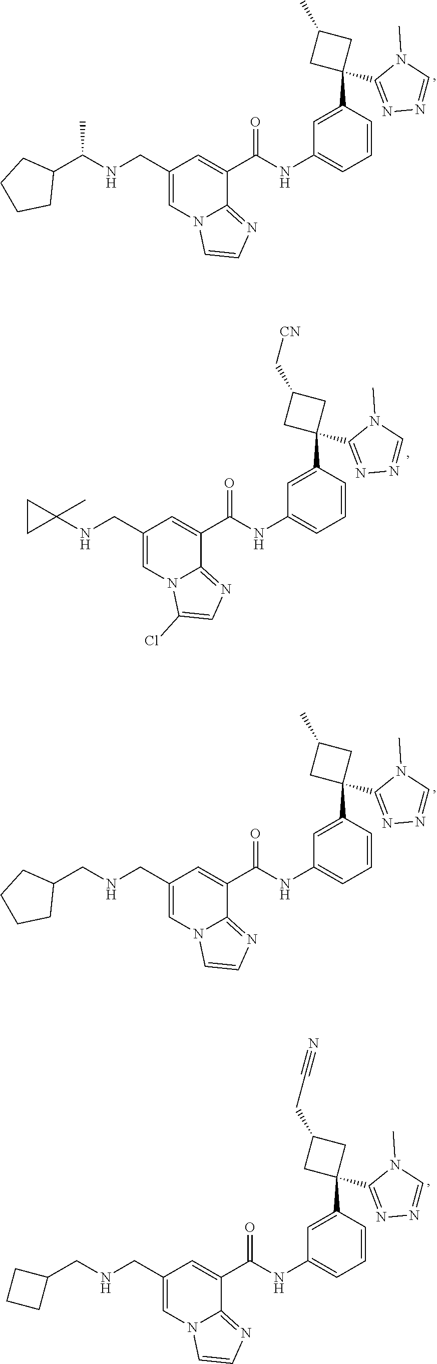 [SMILES: C[C@H](NCc1cc(C(=O)Nc2cccc([C@]3(c4nncn4C)C[C@@H](C)C3)c2)c2nccn2c1)C1CCCC1 ; Cn1cnnc1[C@]1(c2cccc(NC(=O)c3cc(CNC4(C)CC4)cn4c(Cl)cnc34)c2)C[C@H](CC#N)C1 ; Cn1cnnc1[C@]1(c2cccc(NC(=O)c3cc(CNCC4CCCC4)cn4ccnc34)c2)C[C@H](C)C1 ; Cn1cnnc1[C@]1(c2cccc(NC(=O)c3cc(CNCC4CCC4)cn4ccnc34)c2)C[C@H](CC#N)C1] 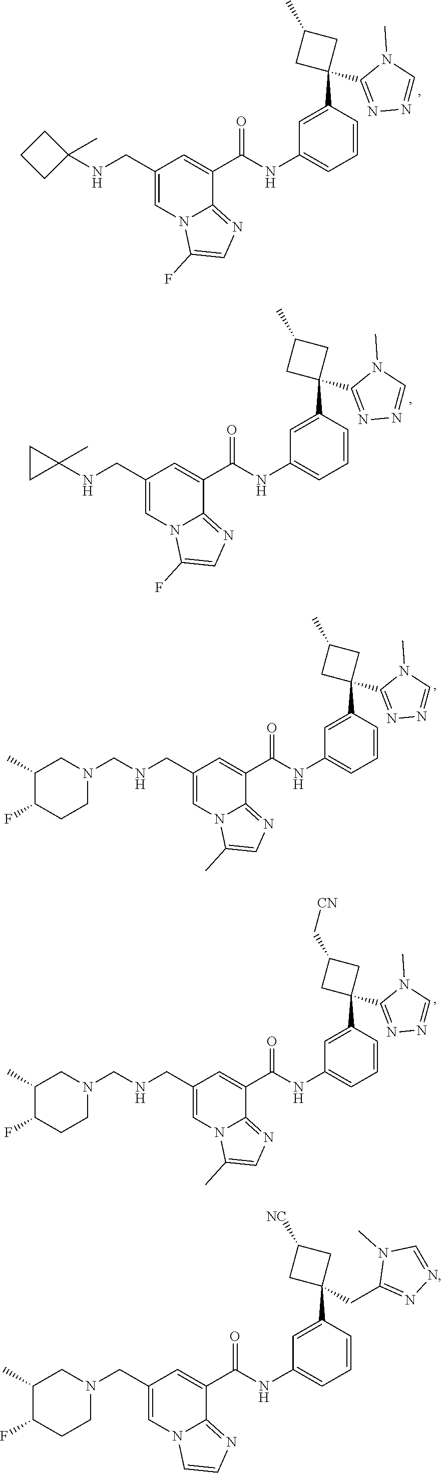 [SMILES: Cn1cnnc1[C@]1(c2cccc(NC(=O)c3cc(CNC4(C)CCC4)cn4c(F)cnc34)c2)C[C@H](C)C1 ; Cn1cnnc1[C@]1(c2cccc(NC(=O)c3cc(CNC4(C)CC4)cn4c(F)cnc34)c2)C[C@H](C)C1 ; Cc1cnc2c(C(=O)Nc3cccc([C@]4(c5nncn5C)C[C@@H](C)C4)c3)cc(CNCN3CC[C@H](F)[C@H](C)C3)cn12 ; Cc1cnc2c(C(=O)Nc3cccc([C@]4(c5nncn5C)C[C@@H](CC#N)C4)c3)cc(CNCN3CC[C@H](F)[C@H](C)C3)cn12 ; C[C@@H]1CN(Cc2cc(C(=O)Nc3cccc([C@]4(Cc5nncn5C)C[C@@H](C#N)C4)c3)c3nccn3c2)CC[C@@H]1F] 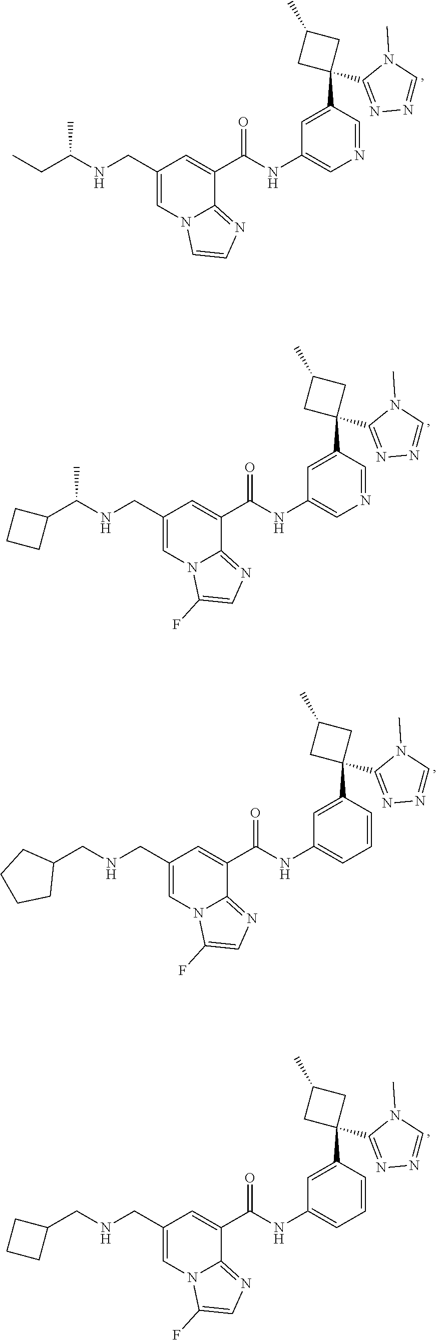 [SMILES: CC[C@H](C)NCc1cc(C(=O)Nc2cncc([C@]3(c4nncn4C)C[C@@H](C)C3)c2)c2nccn2c1 ; C[C@H](NCc1cc(C(=O)Nc2cncc([C@]3(c4nncn4C)C[C@@H](C)C3)c2)c2ncc(F)n2c1)C1CCC1 ; Cn1cnnc1[C@]1(c2cccc(NC(=O)c3cc(CNCC4CCCC4)cn4c(F)cnc34)c2)C[C@H](C)C1 ; Cn1cnnc1[C@]1(c2cccc(NC(=O)c3cc(CNCC4CCC4)cn4c(F)cnc34)c2)C[C@H](C)C1] 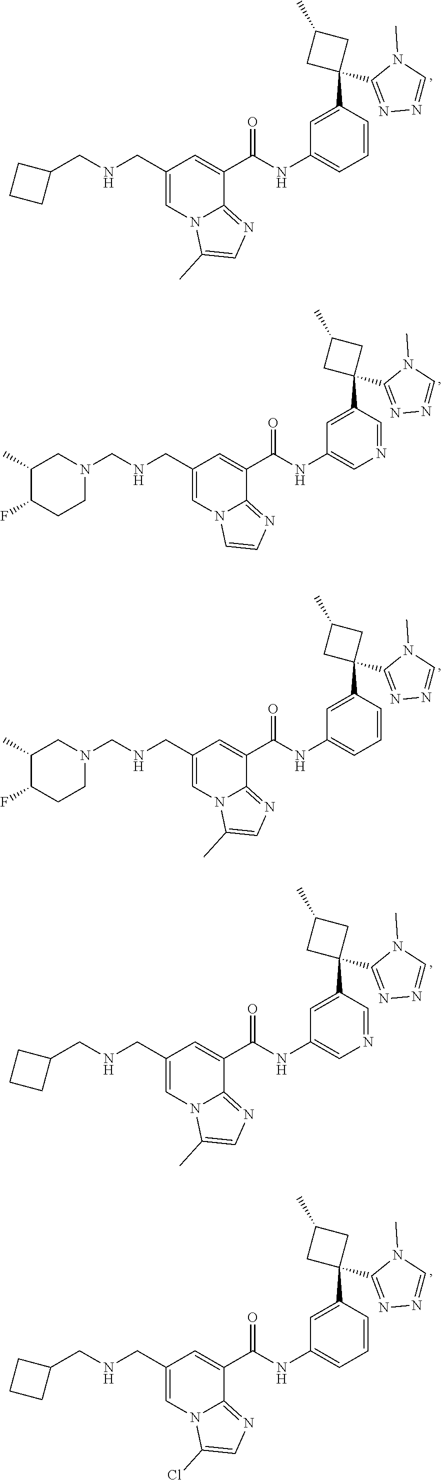 [SMILES: Cc1cnc2c(C(=O)Nc3cccc([C@]4(c5nncn5C)C[C@@H](C)C4)c3)cc(CNCC3CCC3)cn12 ; C[C@@H]1CN(CNCc2cc(C(=O)Nc3cncc([C@]4(c5nncn5C)C[C@@H](C)C4)c3)c3nccn3c2)CC[C@@H]1F ; Cc1cnc2c(C(=O)Nc3cccc([C@]4(c5nncn5C)C[C@@H](C)C4)c3)cc(CNCN3CC[C@H](F)[C@H](C)C3)cn12 ; Cc1cnc2c(C(=O)Nc3cncc([C@]4(c5nncn5C)C[C@@H](C)C4)c3)cc(CNCC3CCC3)cn12 ; Cn1cnnc1[C@]1(c2cccc(NC(=O)c3cc(CNCC4CCC4)cn4c(Cl)cnc34)c2)C[C@H](C)C1] 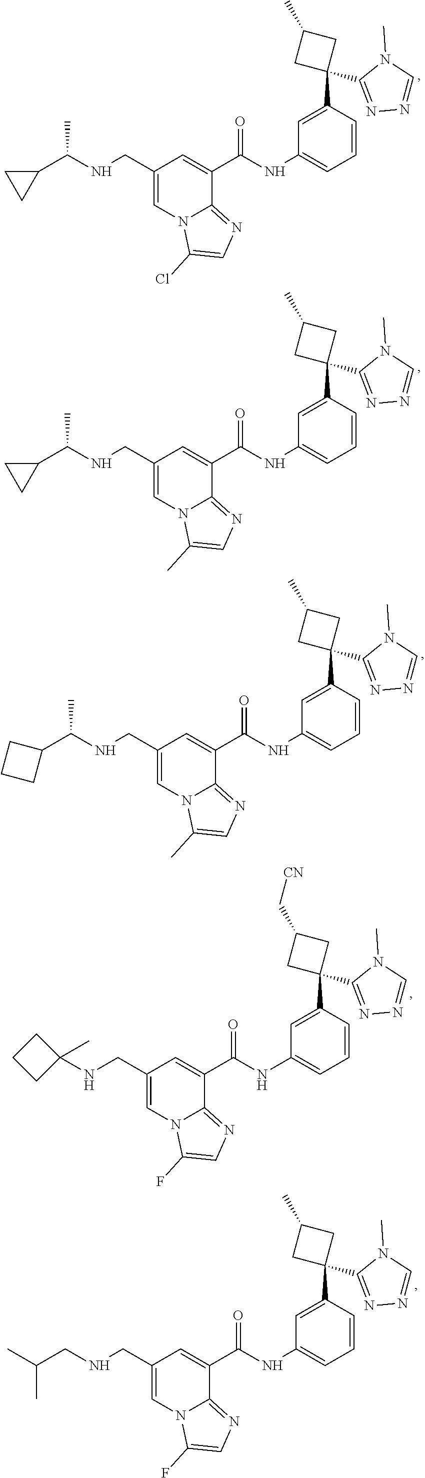 [SMILES: C[C@H](NCc1cc(C(=O)Nc2cccc([C@]3(c4nncn4C)C[C@@H](C)C3)c2)c2ncc(Cl)n2c1)C1CC1 ; Cc1cnc2c(C(=O)Nc3cccc([C@]4(c5nncn5C)C[C@@H](C)C4)c3)cc(CN[C@@H](C)C3CC3)cn12 ; Cc1cnc2c(C(=O)Nc3cccc([C@]4(c5nncn5C)C[C@@H](C)C4)c3)cc(CN[C@@H](C)C3CCC3)cn12 ; Cn1cnnc1[C@]1(c2cccc(NC(=O)c3cc(CNC4(C)CCC4)cn4c(F)cnc34)c2)C[C@H](CC#N)C1 ; CC(C)CNCc1cc(C(=O)Nc2cccc([C@]3(c4nncn4C)C[C@@H](C)C3)c2)c2ncc(F)n2c1] 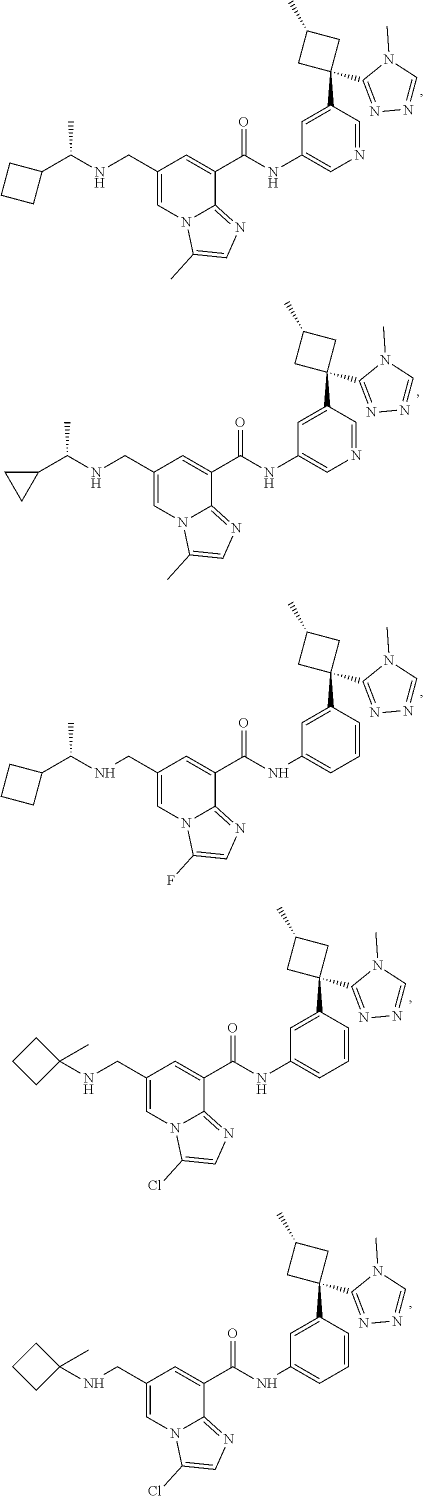 [SMILES: Cc1cnc2c(C(=O)Nc3cncc([C@]4(c5nncn5C)C[C@@H](C)C4)c3)cc(CN[C@@H](C)C3CCC3)cn12 ; Cc1cnc2c(C(=O)Nc3cncc([C@]4(c5nncn5C)C[C@@H](C)C4)c3)cc(CN[C@@H](C)C3CC3)cn12 ; C[C@H](NCc1cc(C(=O)Nc2cccc([C@]3(c4nncn4C)C[C@@H](C)C3)c2)c2ncc(F)n2c1)C1CCC1 ; Cn1cnnc1[C@]1(c2cccc(NC(=O)c3cc(CNC4(C)CCC4)cn4c(Cl)cnc34)c2)C[C@H](C)C1 ; Cn1cnnc1[C@]1(c2cccc(NC(=O)c3cc(CNC4(C)CCC4)cn4c(Cl)cnc34)c2)C[C@H](C)C1] 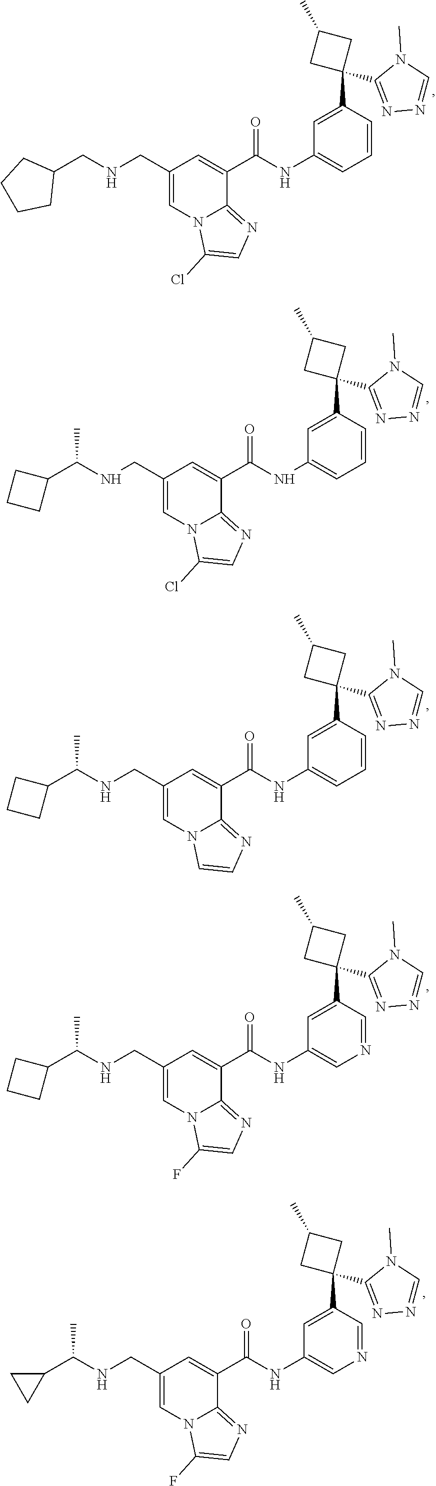 [SMILES: Cn1cnnc1[C@]1(c2cccc(NC(=O)c3cc(CNCC4CCCC4)cn4c(Cl)cnc34)c2)C[C@H](C)C1 ; C[C@H](NCc1cc(C(=O)Nc2cccc([C@]3(c4nncn4C)C[C@@H](C)C3)c2)c2ncc(Cl)n2c1)C1CCC1 ; C[C@H](NCc1cc(C(=O)Nc2cccc([C@]3(c4nncn4C)C[C@@H](C)C3)c2)c2nccn2c1)C1CCC1 ; C[C@H](NCc1cc(C(=O)Nc2cncc([C@]3(c4nncn4C)C[C@@H](C)C3)c2)c2ncc(F)n2c1)C1CCC1 ; C[C@H](NCc1cc(C(=O)Nc2cncc([C@]3(c4nncn4C)C[C@@H](C)C3)c2)c2ncc(F)n2c1)C1CC1] 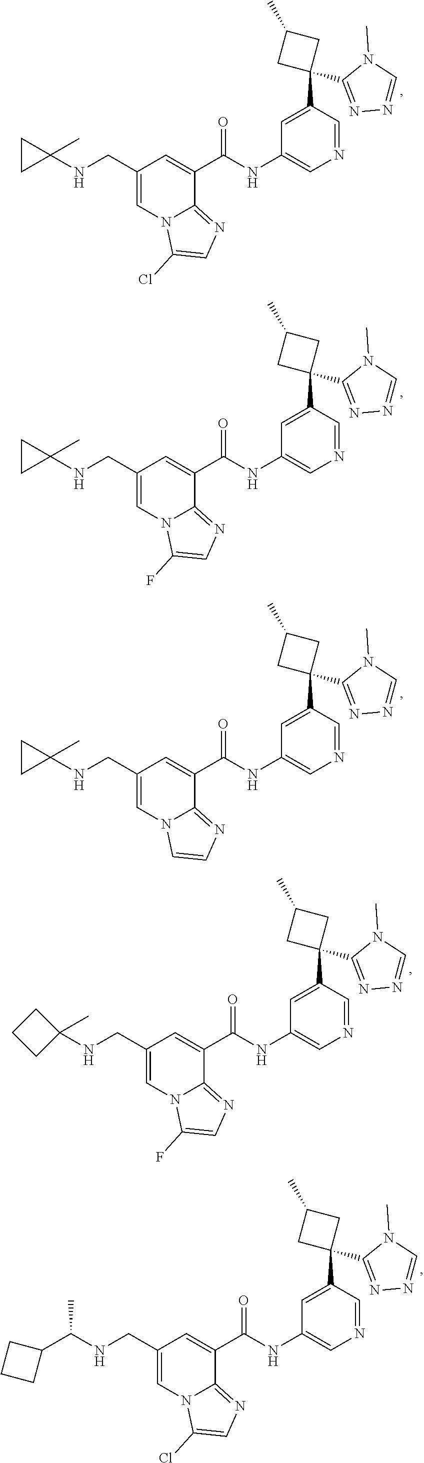 [SMILES: Cn1cnnc1[C@]1(c2cncc(NC(=O)c3cc(CNC4(C)CC4)cn4c(Cl)cnc34)c2)C[C@H](C)C1 ; Cn1cnnc1[C@]1(c2cncc(NC(=O)c3cc(CNC4(C)CC4)cn4c(F)cnc34)c2)C[C@H](C)C1 ; Cn1cnnc1[C@]1(c2cncc(NC(=O)c3cc(CNC4(C)CC4)cn4ccnc34)c2)C[C@H](C)C1 ; Cn1cnnc1[C@]1(c2cncc(NC(=O)c3cc(CNC4(C)CCC4)cn4c(F)cnc34)c2)C[C@H](C)C1 ; C[C@H](NCc1cc(C(=O)Nc2cncc([C@]3(c4nncn4C)C[C@@H](C)C3)c2)c2ncc(Cl)n2c1)C1CCC1] 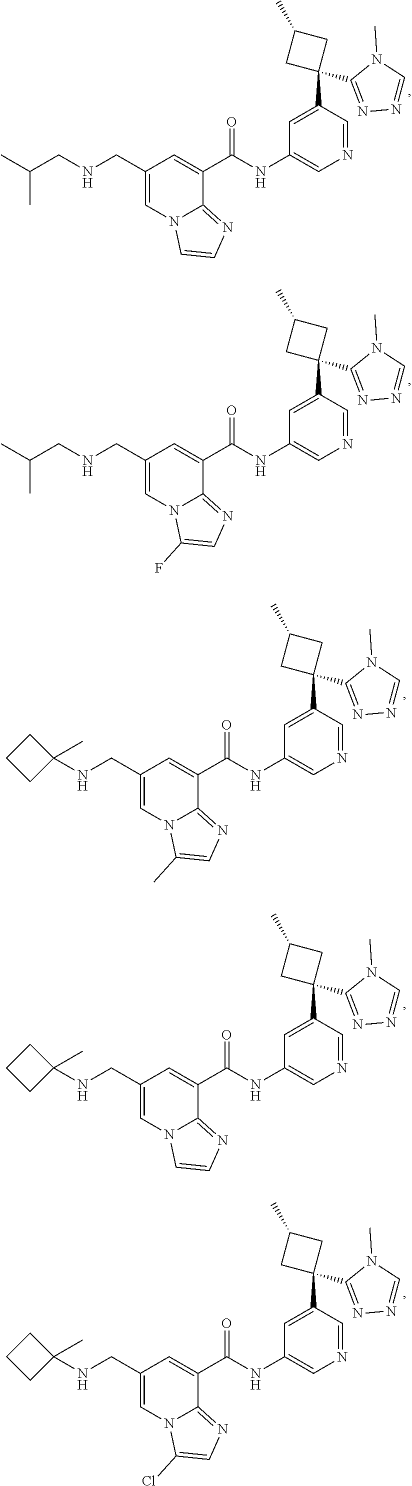 [SMILES: CC(C)CNCc1cc(C(=O)Nc2cncc([C@]3(c4nncn4C)C[C@@H](C)C3)c2)c2nccn2c1 ; CC(C)CNCc1cc(C(=O)Nc2cncc([C@]3(c4nncn4C)C[C@@H](C)C3)c2)c2ncc(F)n2c1 ; Cc1cnc2c(C(=O)Nc3cncc([C@]4(c5nncn5C)C[C@@H](C)C4)c3)cc(CNC3(C)CCC3)cn12 ; Cn1cnnc1[C@]1(c2cncc(NC(=O)c3cc(CNC4(C)CCC4)cn4ccnc34)c2)C[C@H](C)C1 ; Cn1cnnc1[C@]1(c2cncc(NC(=O)c3cc(CNC4(C)CCC4)cn4c(Cl)cnc34)c2)C[C@H](C)C1] 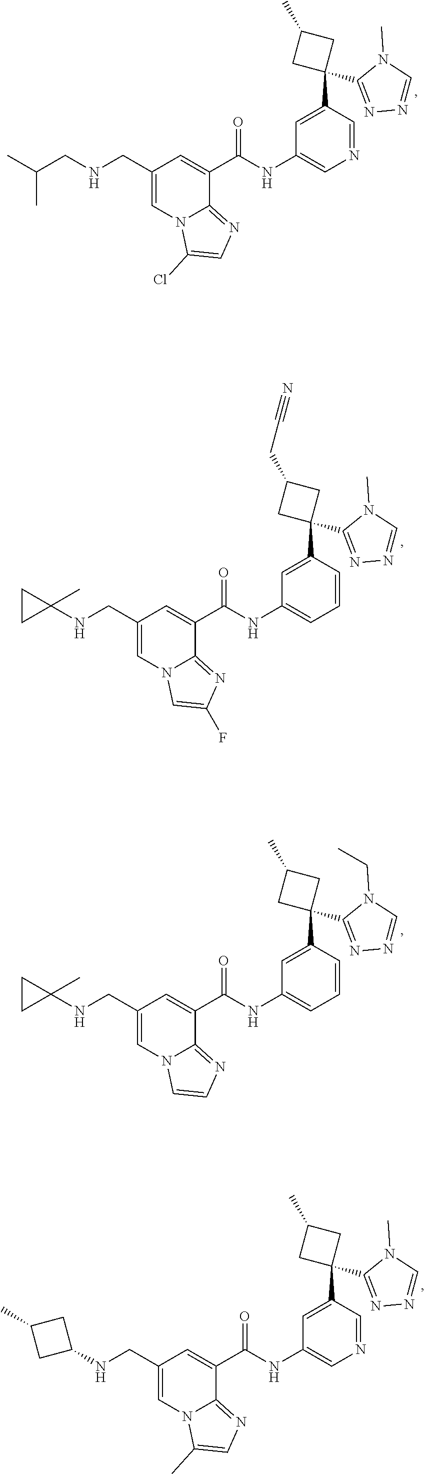 [SMILES: CC(C)CNCc1cc(C(=O)Nc2cncc([C@]3(c4nncn4C)C[C@@H](C)C3)c2)c2ncc(Cl)n2c1 ; Cn1cnnc1[C@]1(c2cccc(NC(=O)c3cc(CNC4(C)CC4)cn4cc(F)nc34)c2)C[C@H](CC#N)C1 ; CCn1cnnc1[C@]1(c2cccc(NC(=O)c3cc(CNC4(C)CC4)cn4ccnc34)c2)C[C@H](C)C1 ; Cc1cnc2c(C(=O)Nc3cncc([C@]4(c5nncn5C)C[C@@H](C)C4)c3)cc(CN[C@H]3C[C@@H](C)C3)cn12] 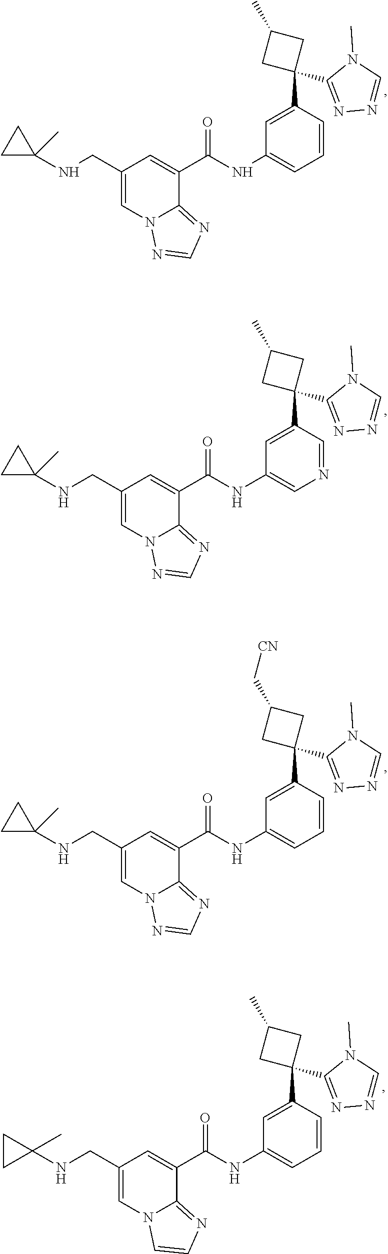 [SMILES: Cn1cnnc1[C@]1(c2cccc(NC(=O)c3cc(CNC4(C)CC4)cn4ncnc34)c2)C[C@H](C)C1 ; Cn1cnnc1[C@]1(c2cncc(NC(=O)c3cc(CNC4(C)CC4)cn4ncnc34)c2)C[C@H](C)C1 ; Cn1cnnc1[C@]1(c2cccc(NC(=O)c3cc(CNC4(C)CC4)cn4ncnc34)c2)C[C@H](CC#N)C1 ; Cn1cnnc1[C@]1(c2cccc(NC(=O)c3cc(CNC4(C)CC4)cn4ccnc34)c2)C[C@H](C)C1] 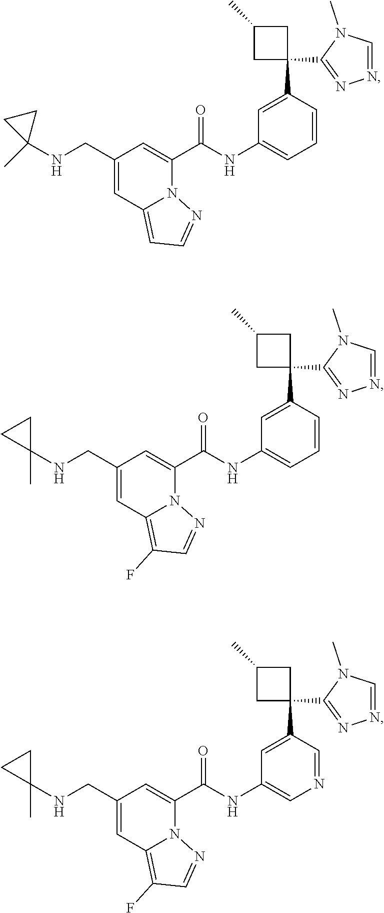 [SMILES: Cn1cnnc1[C@]1(c2cccc(NC(=O)c3cc(CNC4(C)CC4)cc4ccnn34)c2)C[C@H](C)C1 ; Cn1cnnc1[C@]1(c2cccc(NC(=O)c3cc(CNC4(C)CC4)cc4c(F)cnn34)c2)C[C@H](C)C1 ; Cn1cnnc1[C@]1(c2cncc(NC(=O)c3cc(CNC4(C)CC4)cc4c(F)cnn34)c2)C[C@H](C)C1] 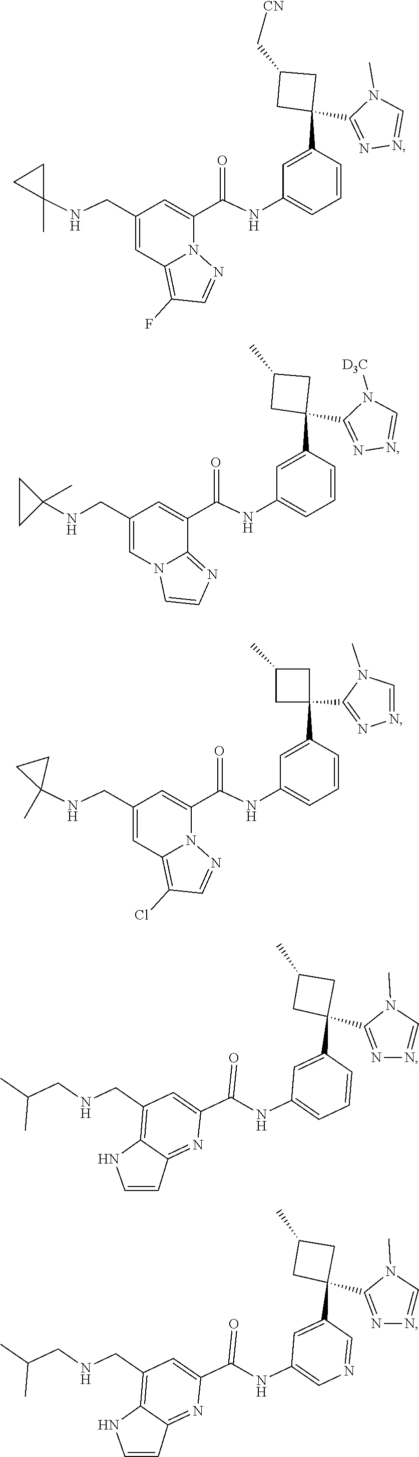 [SMILES: Cn1cnnc1[C@]1(c2cccc(NC(=O)c3cc(CNC4(C)CC4)cc4c(F)cnn34)c2)C[C@H](CC#N)C1 ; [2H]C([2H])([2H])n1cnnc1[C@]1(c2cccc(NC(=O)c3cc(CNC4(C)CC4)cn4ccnc34)c2)C[C@H](C)C1 ; Cn1cnnc1[C@]1(c2cccc(NC(=O)c3cc(CNC4(C)CC4)cc4c(Cl)cnn34)c2)C[C@H](C)C1 ; CC(C)CNCc1cc(C(=O)Nc2cccc([C@]3(c4nncn4C)C[C@@H](C)C3)c2)nc2cc[nH]c12 ; CC(C)CNCc1cc(C(=O)Nc2cncc([C@]3(c4nncn4C)C[C@@H](C)C3)c2)nc2cc[nH]c12] 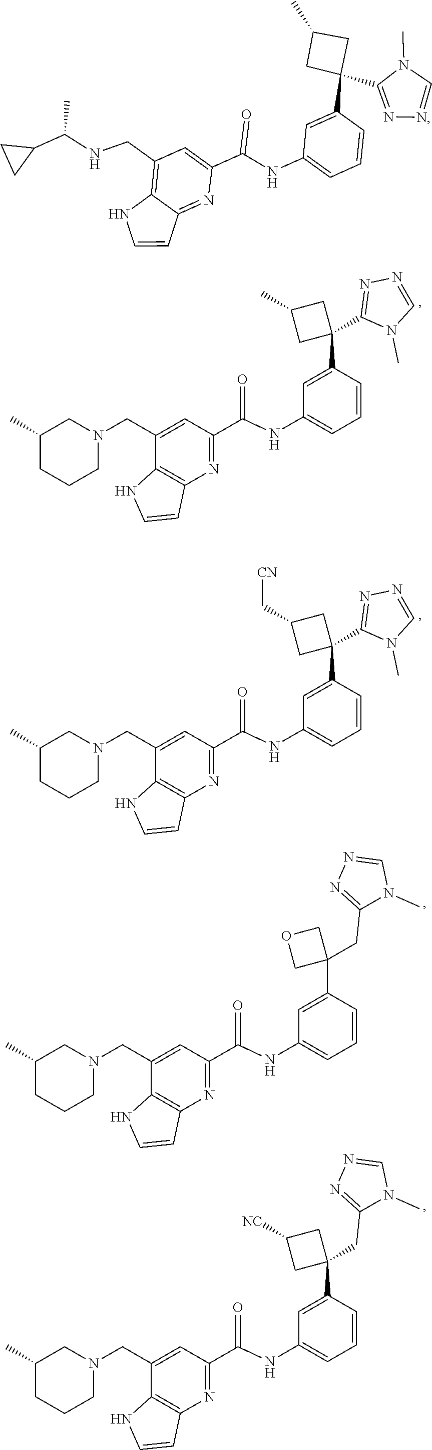 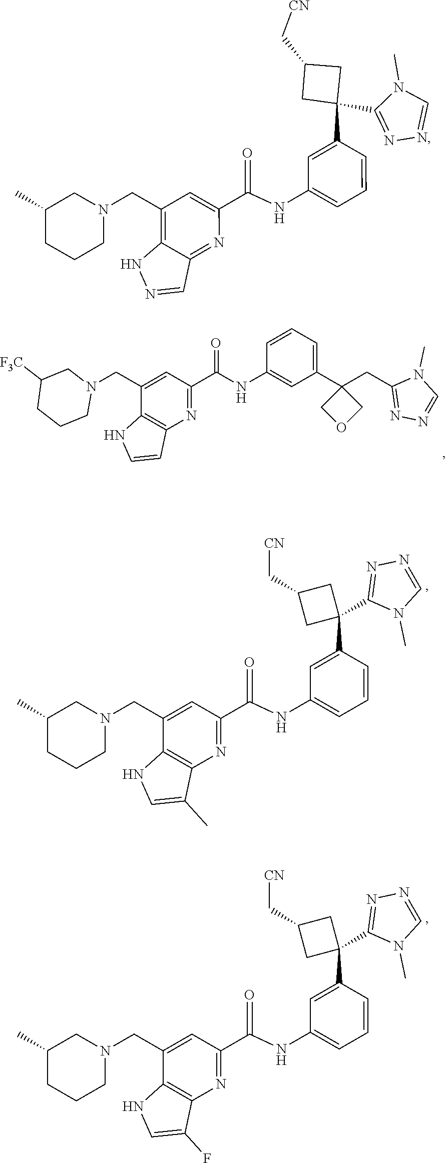 [SMILES: C[C@H]1CCCN(Cc2cc(C(=O)Nc3cccc([C@]4(c5nncn5C)C[C@@H](CC#N)C4)c3)nc3cn[nH]c23)C1 ; Cn1cnnc1CC1(c2cccc(NC(=O)c3cc(CN4CCCC(C(F)(F)F)C4)c4[nH]ccc4n3)c2)COC1 ; Cc1c[nH]c2c(CN3CCC[C@H](C)C3)cc(C(=O)Nc3cccc([C@]4(c5nncn5C)C[C@@H](CC#N)C4)c3)nc12 ; C[C@H]1CCCN(Cc2cc(C(=O)Nc3cccc([C@]4(c5nncn5C)C[C@@H](CC#N)C4)c3)nc3c(F)c[nH]c23)C1] 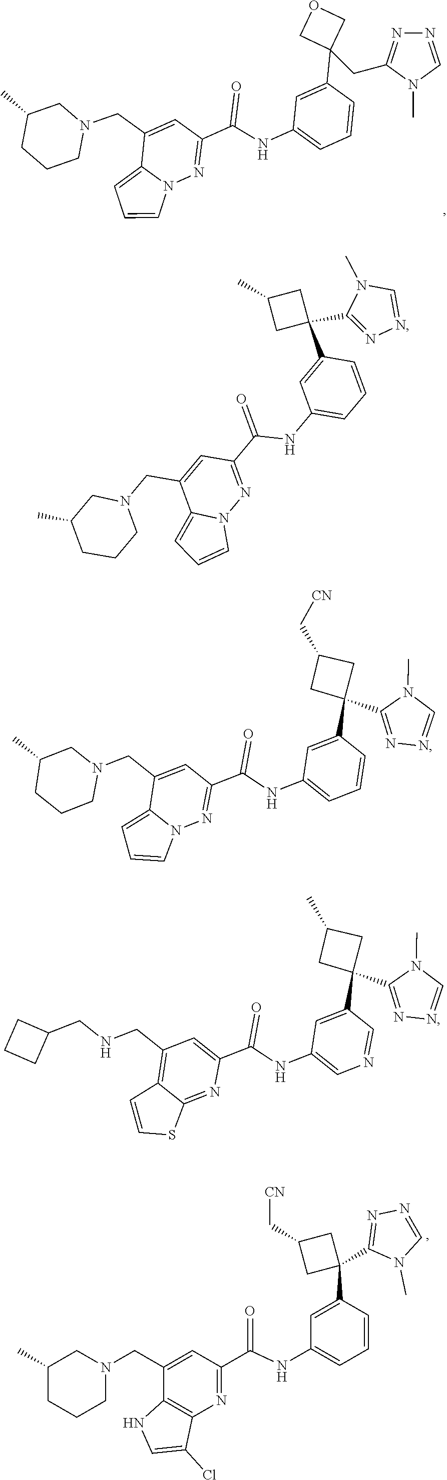 [SMILES: C[C@H]1CCCN(Cc2cc(C(=O)Nc3cccc(C4(Cc5nncn5C)COC4)c3)nn3cccc23)C1 ; C[C@H]1CCCN(Cc2cc(C(=O)Nc3cccc([C@]4(c5nncn5C)C[C@@H](C)C4)c3)nn3cccc23)C1 ; C[C@H]1CCCN(Cc2cc(C(=O)Nc3cccc([C@]4(c5nncn5C)C[C@@H](CC#N)C4)c3)nn3cccc23)C1 ; Cn1cnnc1[C@]1(c2cncc(NC(=O)c3cc(CNCC4CCC4)c4ccsc4n3)c2)C[C@H](C)C1 ; C[C@H]1CCCN(Cc2cc(C(=O)Nc3cccc([C@]4(c5nncn5C)C[C@@H](CC#N)C4)c3)nc3c(Cl)c[nH]c23)C1] 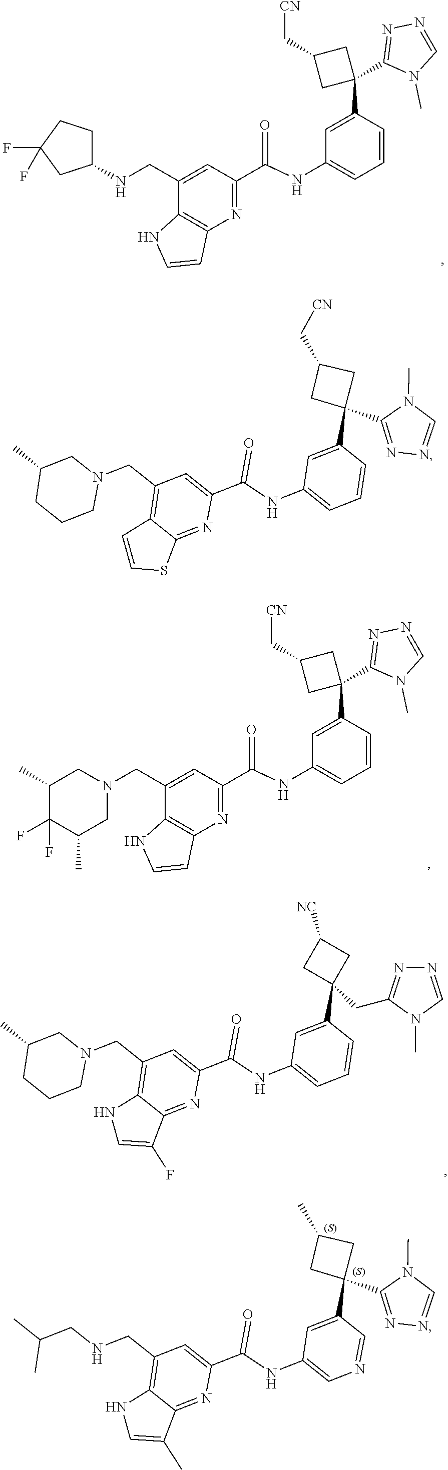 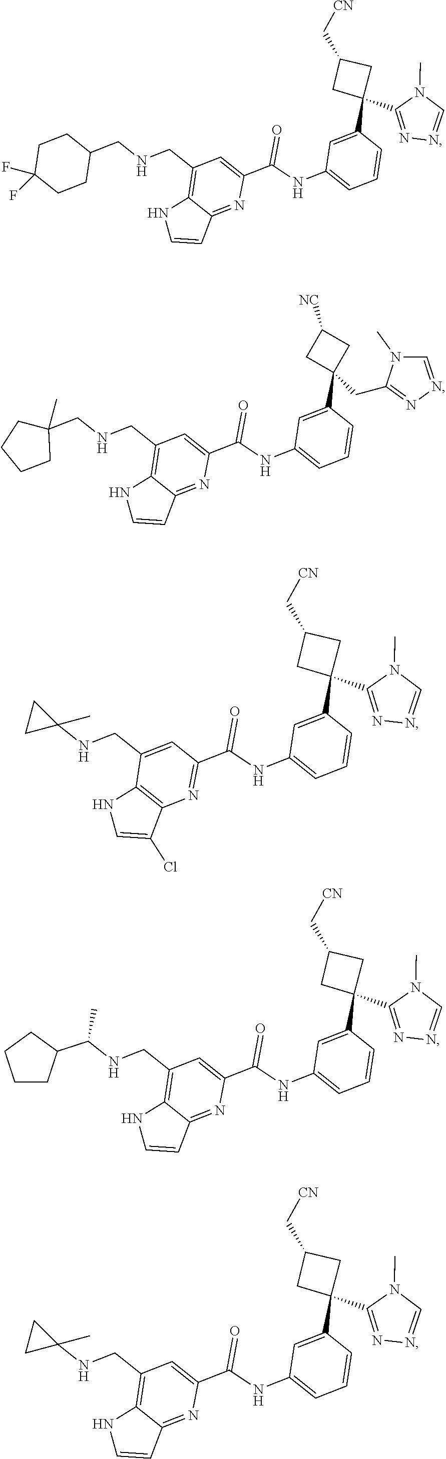 [SMILES: Cn1cnnc1[C@]1(c2cccc(NC(=O)c3cc(CNCC4CCC(F)(F)CC4)c4[nH]ccc4n3)c2)C[C@H](CC#N)C1 ; Cn1cnnc1C[C@]1(c2cccc(NC(=O)c3cc(CNCC4(C)CCCC4)c4[nH]ccc4n3)c2)C[C@H](C#N)C1 ; Cn1cnnc1[C@]1(c2cccc(NC(=O)c3cc(CNC4(C)CC4)c4[nH]cc(Cl)c4n3)c2)C[C@H](CC#N)C1 ; C[C@H](NCc1cc(C(=O)Nc2cccc([C@]3(c4nncn4C)C[C@@H](CC#N)C3)c2)nc2cc[nH]c12)C1CCCC1 ; Cn1cnnc1[C@]1(c2cccc(NC(=O)c3cc(CNC4(C)CC4)c4[nH]ccc4n3)c2)C[C@H](CC#N)C1] 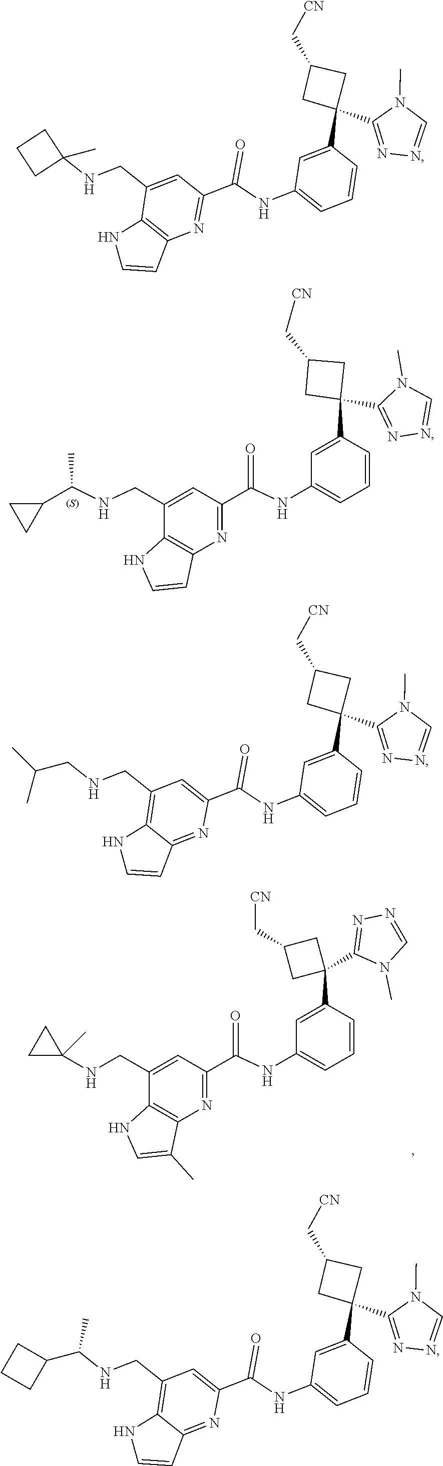 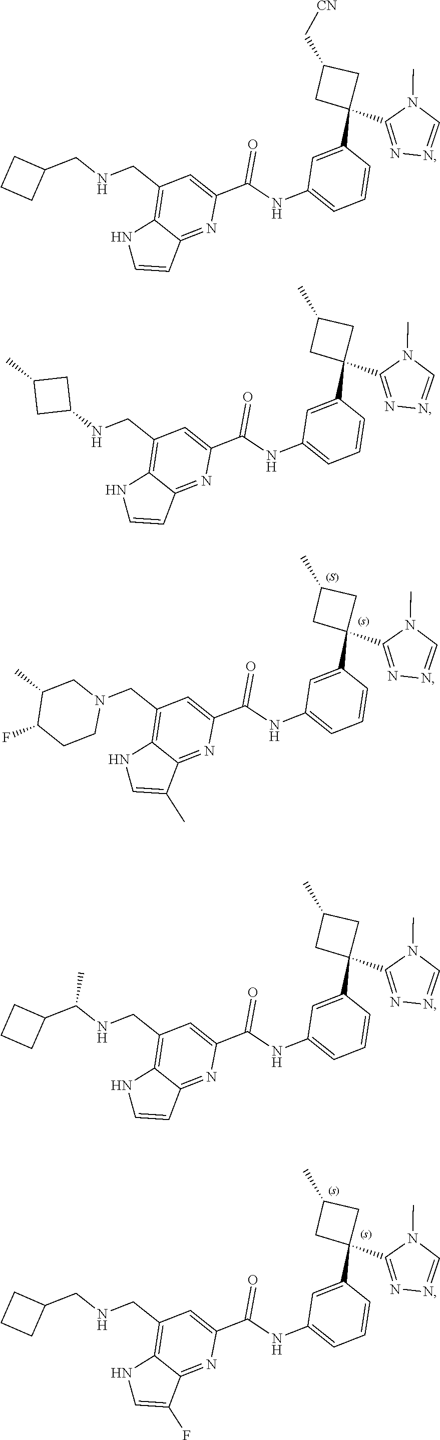 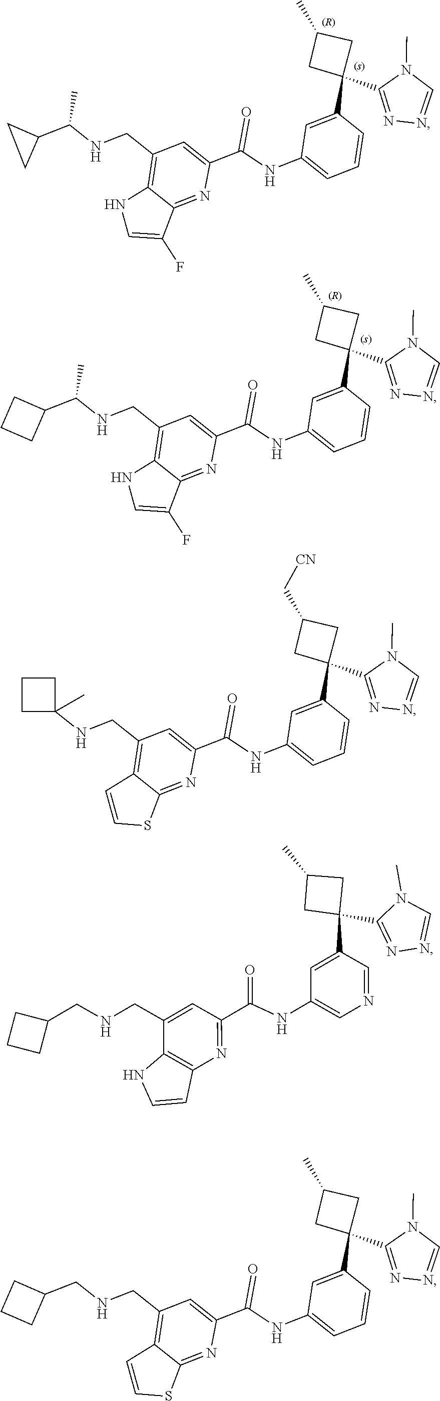 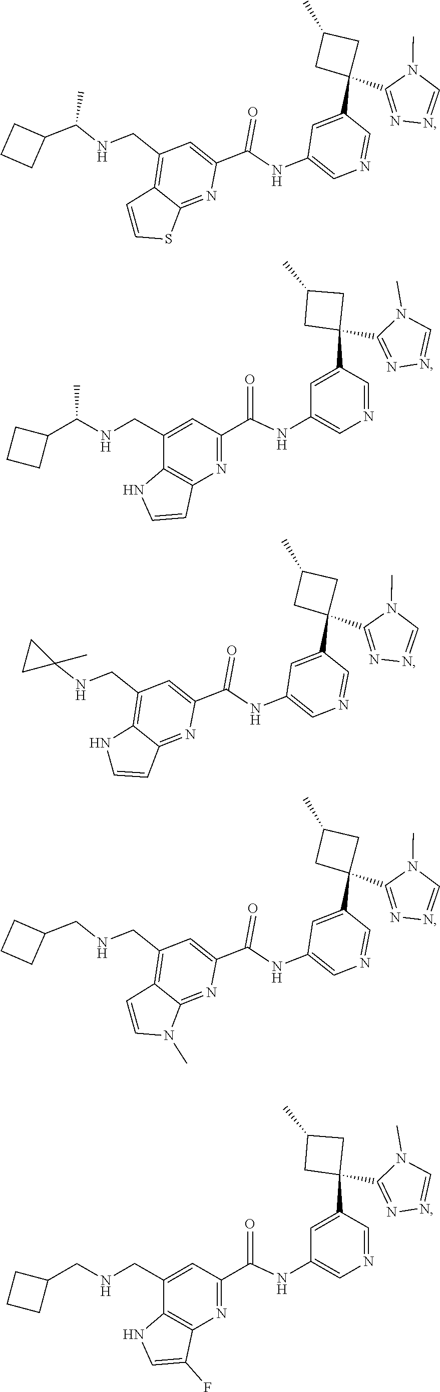 [SMILES: C[C@H](NCc1cc(C(=O)Nc2cncc([C@]3(c4nncn4C)C[C@@H](C)C3)c2)nc2sccc12)C1CCC1 ; C[C@H](NCc1cc(C(=O)Nc2cncc([C@]3(c4nncn4C)C[C@@H](C)C3)c2)nc2cc[nH]c12)C1CCC1 ; Cn1cnnc1[C@]1(c2cncc(NC(=O)c3cc(CNC4(C)CC4)c4[nH]ccc4n3)c2)C[C@H](C)C1 ; Cn1ccc2c(CNCC3CCC3)cc(C(=O)Nc3cncc([C@]4(c5nncn5C)C[C@@H](C)C4)c3)nc21 ; Cn1cnnc1[C@]1(c2cncc(NC(=O)c3cc(CNCC4CCC4)c4[nH]cc(F)c4n3)c2)C[C@H](C)C1] 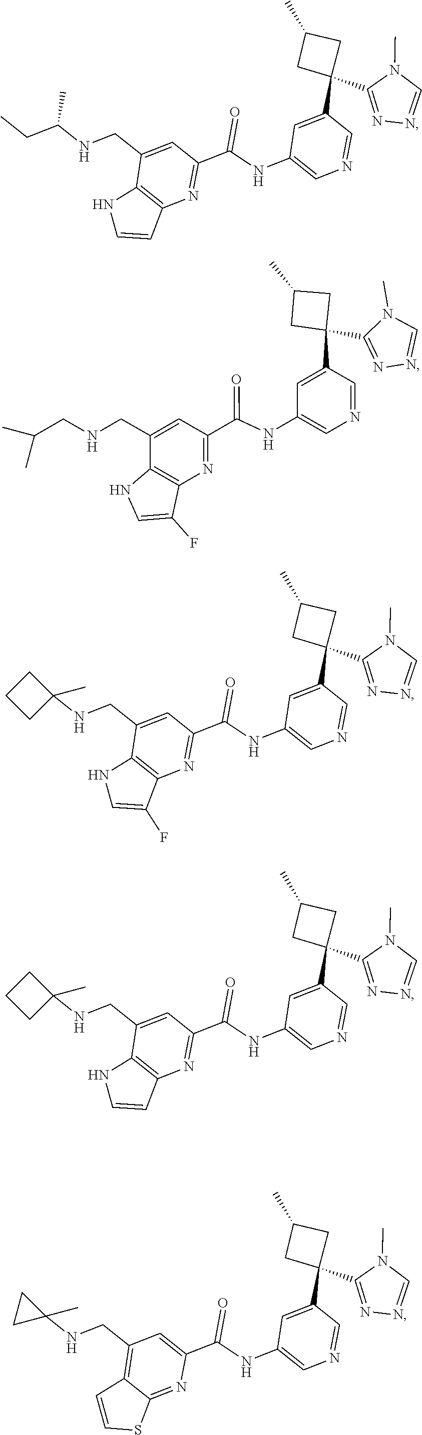 [SMILES: CC[C@H](C)NCc1cc(C(=O)Nc2cncc([C@]3(c4nncn4C)C[C@@H](C)C3)c2)nc2cc[nH]c12 ; CC(C)CNCc1cc(C(=O)Nc2cncc([C@]3(c4nncn4C)C[C@@H](C)C3)c2)nc2c(F)c[nH]c12 ; Cn1cnnc1[C@]1(c2cncc(NC(=O)c3cc(CNC4(C)CCC4)c4[nH]cc(F)c4n3)c2)C[C@H](C)C1 ; Cn1cnnc1[C@]1(c2cncc(NC(=O)c3cc(CNC4(C)CCC4)c4[nH]ccc4n3)c2)C[C@H](C)C1 ; Cn1cnnc1[C@]1(c2cncc(NC(=O)c3cc(CNC4(C)CC4)c4ccsc4n3)c2)C[C@H](C)C1] 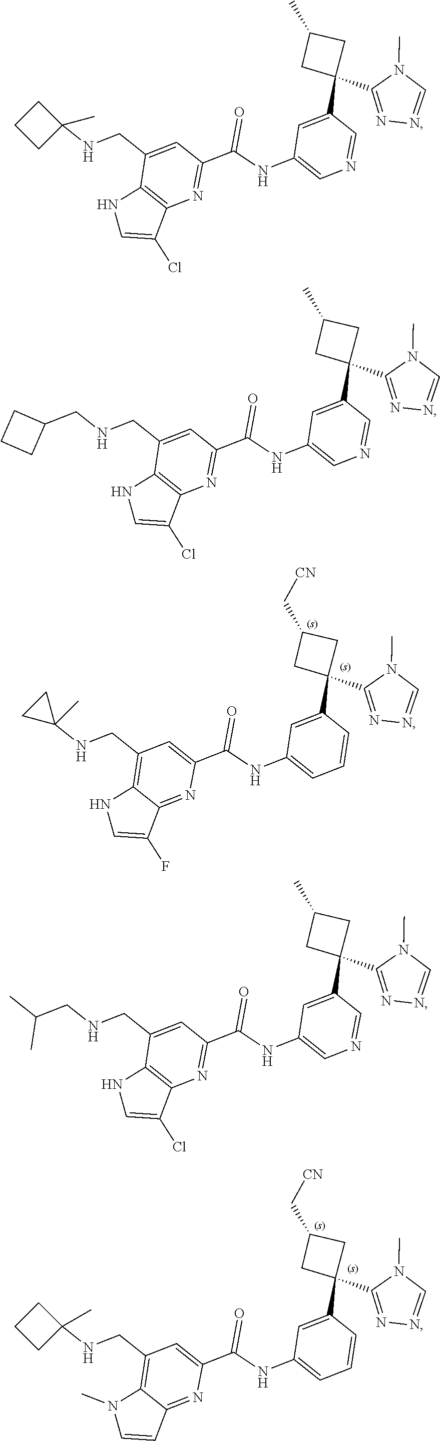 [SMILES: Cn1cnnc1[C@]1(c2cncc(NC(=O)c3cc(CNC4(C)CCC4)c4[nH]cc(Cl)c4n3)c2)C[C@H](C)C1 ; Cn1cnnc1[C@]1(c2cncc(NC(=O)c3cc(CNCC4CCC4)c4[nH]cc(Cl)c4n3)c2)C[C@H](C)C1 ; Cn1cnnc1[C@]1(c2cccc(NC(=O)c3cc(CNC4(C)CC4)c4[nH]cc(F)c4n3)c2)C[C@H](CC#N)C1 ; CC(C)CNCc1cc(C(=O)Nc2cncc([C@]3(c4nncn4C)C[C@@H](C)C3)c2)nc2c(Cl)c[nH]c12 ; Cn1ccc2nc(C(=O)Nc3cccc([C@]4(c5nncn5C)C[C@@H](CC#N)C4)c3)cc(CNC3(C)CCC3)c21] 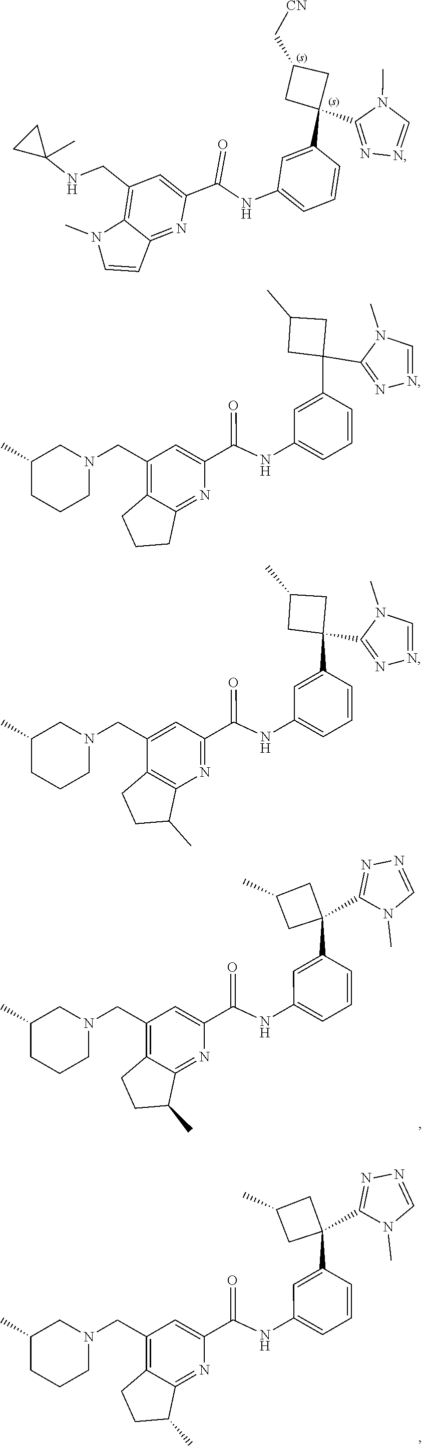 [SMILES: Cn1ccc2nc(C(=O)Nc3cccc([C@]4(c5nncn5C)C[C@@H](CC#N)C4)c3)cc(CNC3(C)CC3)c21 ; CC1CC(c2cccc(NC(=O)c3cc(CN4CCC[C@H](C)C4)c4c(n3)CCC4)c2)(c2nncn2C)C1 ; CC1CCc2c(CN3CCC[C@H](C)C3)cc(C(=O)Nc3cccc([C@]4(c5nncn5C)C[C@@H](C)C4)c3)nc21 ; C[C@H]1CCCN(Cc2cc(C(=O)Nc3cccc([C@]4(c5nncn5C)C[C@@H](C)C4)c3)nc3c2CC[C@@H]3C)C1 ; C[C@H]1CCCN(Cc2cc(C(=O)Nc3cccc([C@]4(c5nncn5C)C[C@@H](C)C4)c3)nc3c2CC[C@H]3C)C1] 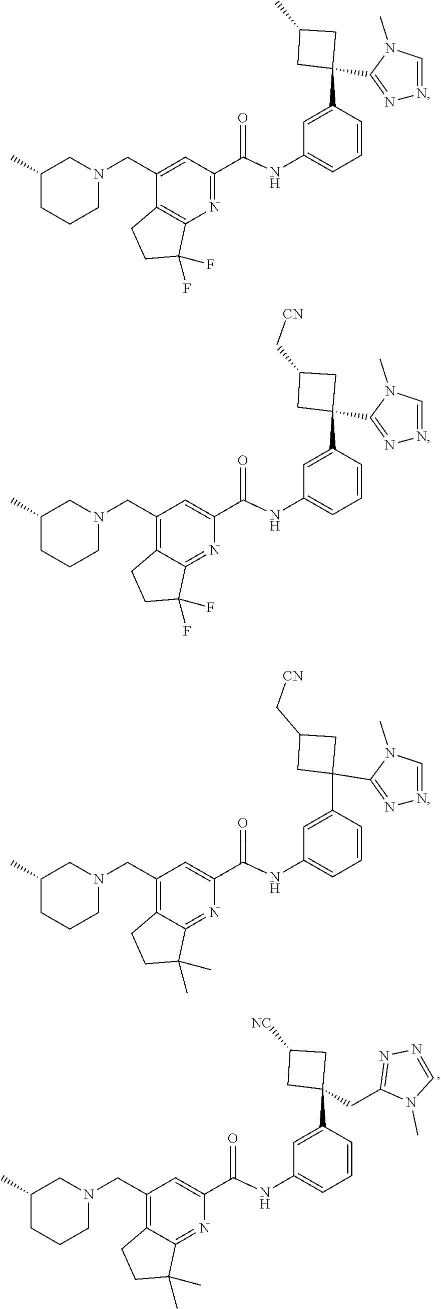 [SMILES: C[C@H]1CCCN(Cc2cc(C(=O)Nc3cccc([C@]4(c5nncn5C)C[C@@H](C)C4)c3)nc3c2CCC3(F)F)C1 ; C[C@H]1CCCN(Cc2cc(C(=O)Nc3cccc([C@]4(c5nncn5C)C[C@@H](CC#N)C4)c3)nc3c2CCC3(F)F)C1 ; C[C@H]1CCCN(Cc2cc(C(=O)Nc3cccc(C4(c5nncn5C)CC(CC#N)C4)c3)nc3c2CCC3(C)C)C1 ; C[C@H]1CCCN(Cc2cc(C(=O)Nc3cccc([C@]4(Cc5nncn5C)C[C@@H](C#N)C4)c3)nc3c2CCC3(C)C)C1] 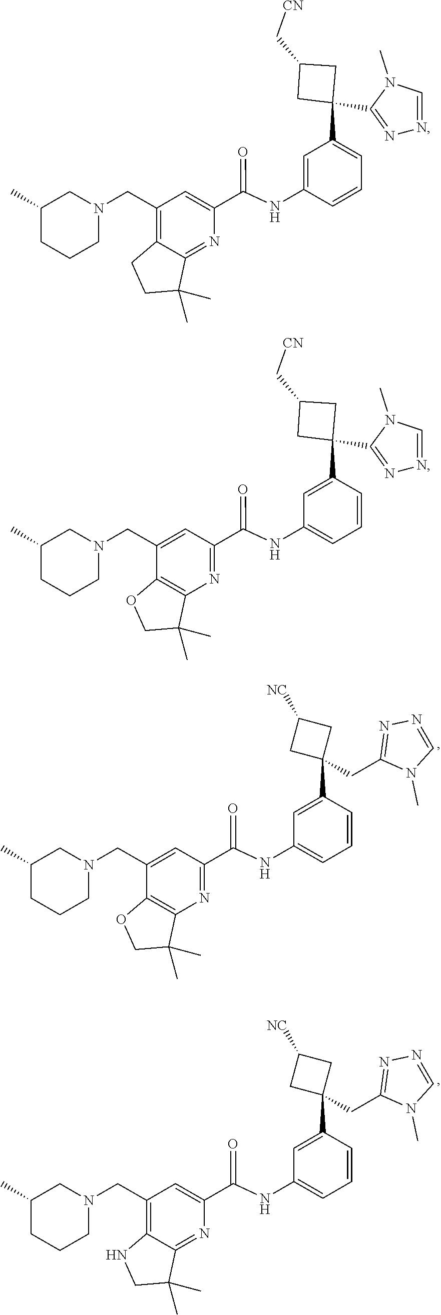 [SMILES: C[C@H]1CCCN(Cc2cc(C(=O)Nc3cccc([C@]4(c5nncn5C)C[C@@H](CC#N)C4)c3)nc3c2CCC3(C)C)C1 ; C[C@H]1CCCN(Cc2cc(C(=O)Nc3cccc([C@]4(c5nncn5C)C[C@@H](CC#N)C4)c3)nc3c2OCC3(C)C)C1 ; C[C@H]1CCCN(Cc2cc(C(=O)Nc3cccc([C@]4(Cc5nncn5C)C[C@@H](C#N)C4)c3)nc3c2OCC3(C)C)C1 ; C[C@H]1CCCN(Cc2cc(C(=O)Nc3cccc([C@]4(Cc5nncn5C)C[C@@H](C#N)C4)c3)nc3c2NCC3(C)C)C1] 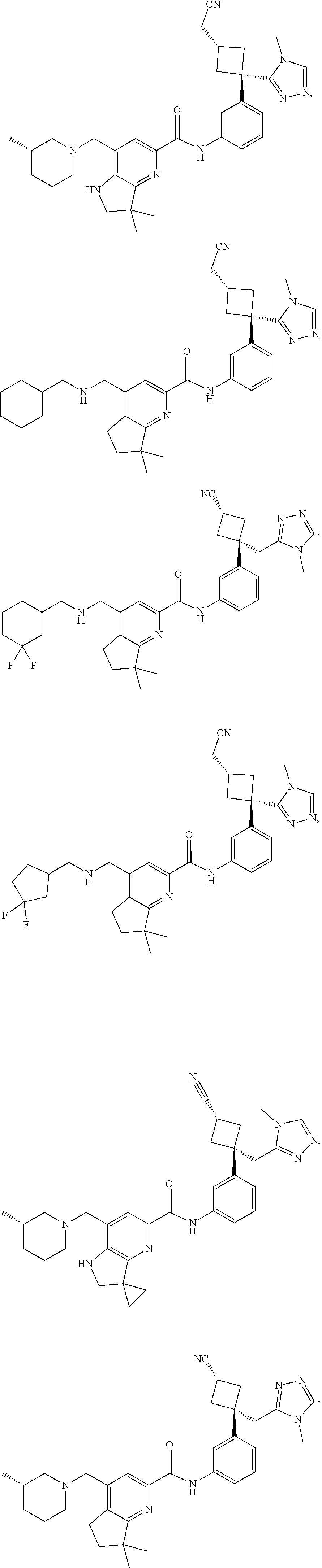 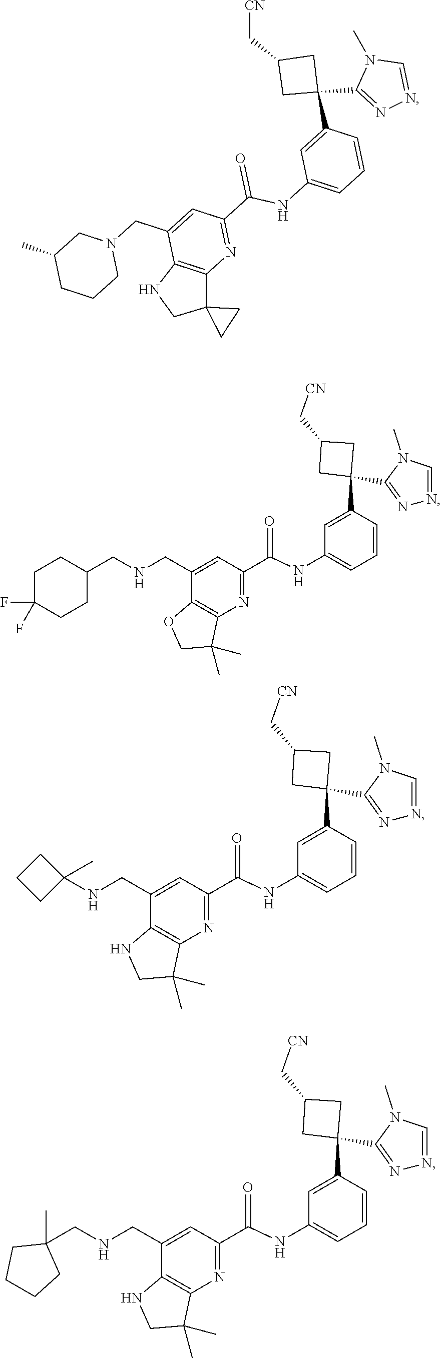 [SMILES: C[C@H]1CCCN(Cc2cc(C(=O)Nc3cccc([C@]4(c5nncn5C)C[C@@H](CC#N)C4)c3)nc3c2NCC32CC2)C1 ; Cn1cnnc1[C@]1(c2cccc(NC(=O)c3cc(CNCC4CCC(F)(F)CC4)c4c(n3)C(C)(C)CO4)c2)C[C@H](CC#N)C1 ; Cn1cnnc1[C@]1(c2cccc(NC(=O)c3cc(CNC4(C)CCC4)c4c(n3)C(C)(C)CN4)c2)C[C@H](CC#N)C1 ; Cn1cnnc1[C@]1(c2cccc(NC(=O)c3cc(CNCC4(C)CCCC4)c4c(n3)C(C)(C)CN4)c2)C[C@H](CC#N)C1] 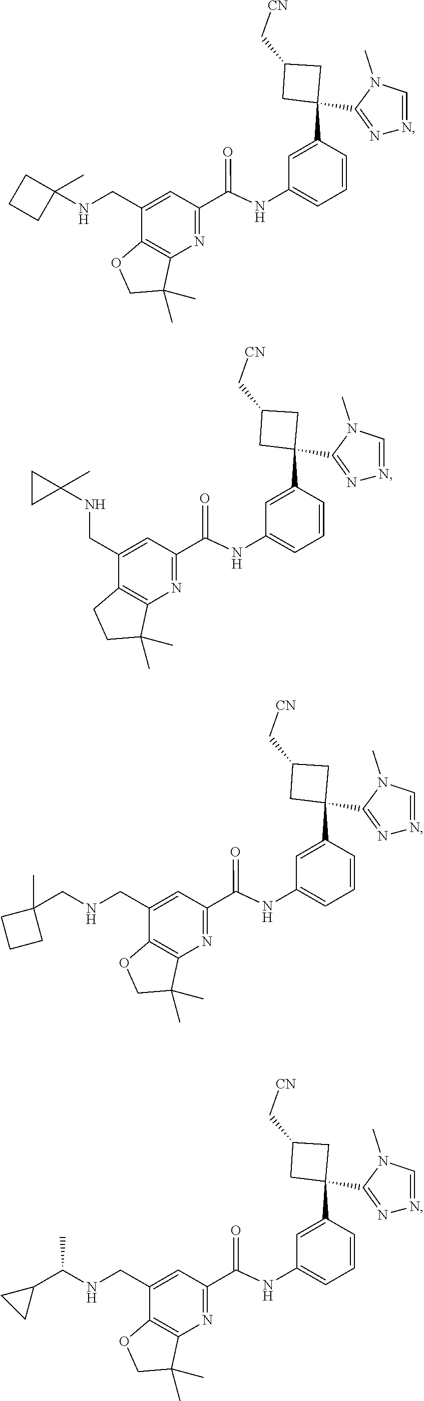 [SMILES: Cn1cnnc1[C@]1(c2cccc(NC(=O)c3cc(CNC4(C)CCC4)c4c(n3)C(C)(C)CO4)c2)C[C@H](CC#N)C1 ; Cn1cnnc1[C@]1(c2cccc(NC(=O)c3cc(CNC4(C)CC4)c4c(n3)C(C)(C)CC4)c2)C[C@H](CC#N)C1 ; Cn1cnnc1[C@]1(c2cccc(NC(=O)c3cc(CNCC4(C)CCC4)c4c(n3)C(C)(C)CO4)c2)C[C@H](CC#N)C1 ; C[C@H](NCc1cc(C(=O)Nc2cccc([C@]3(c4nncn4C)C[C@@H](CC#N)C3)c2)nc2c1OCC2(C)C)C1CC1] 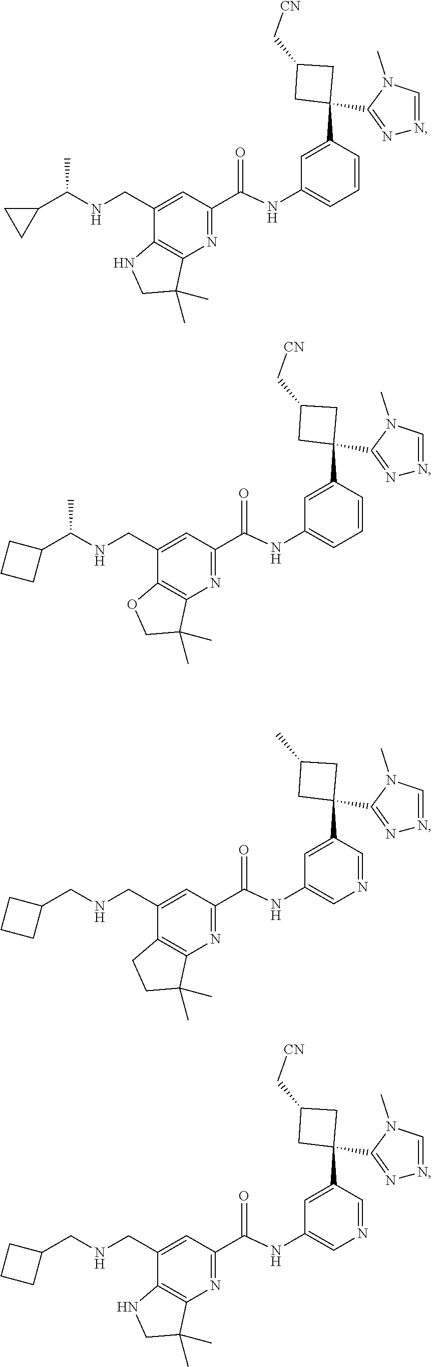 [SMILES: C[C@H](NCc1cc(C(=O)Nc2cccc([C@]3(c4nncn4C)C[C@@H](CC#N)C3)c2)nc2c1NCC2(C)C)C1CC1 ; C[C@H](NCc1cc(C(=O)Nc2cccc([C@]3(c4nncn4C)C[C@@H](CC#N)C3)c2)nc2c1OCC2(C)C)C1CCC1 ; Cn1cnnc1[C@]1(c2cncc(NC(=O)c3cc(CNCC4CCC4)c4c(n3)C(C)(C)CC4)c2)C[C@H](C)C1 ; Cn1cnnc1[C@]1(c2cncc(NC(=O)c3cc(CNCC4CCC4)c4c(n3)C(C)(C)CN4)c2)C[C@H](CC#N)C1] 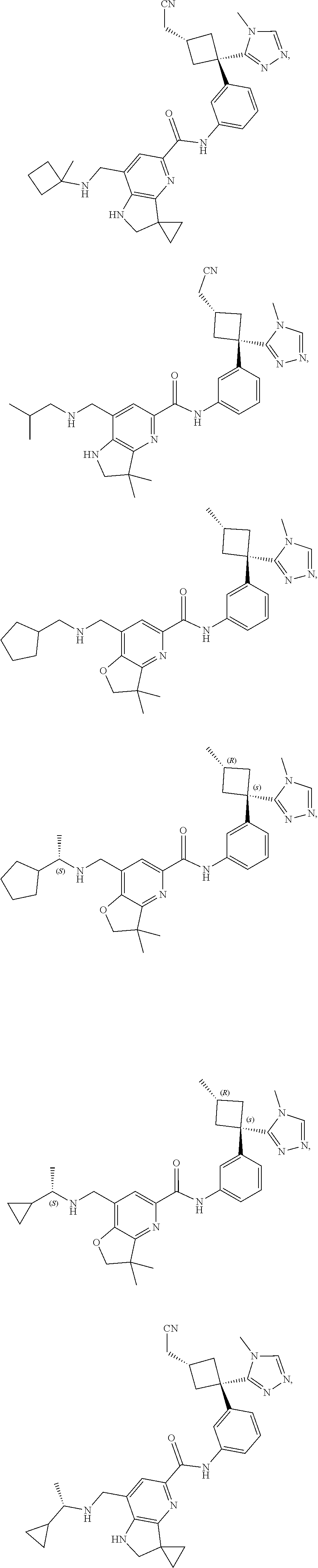 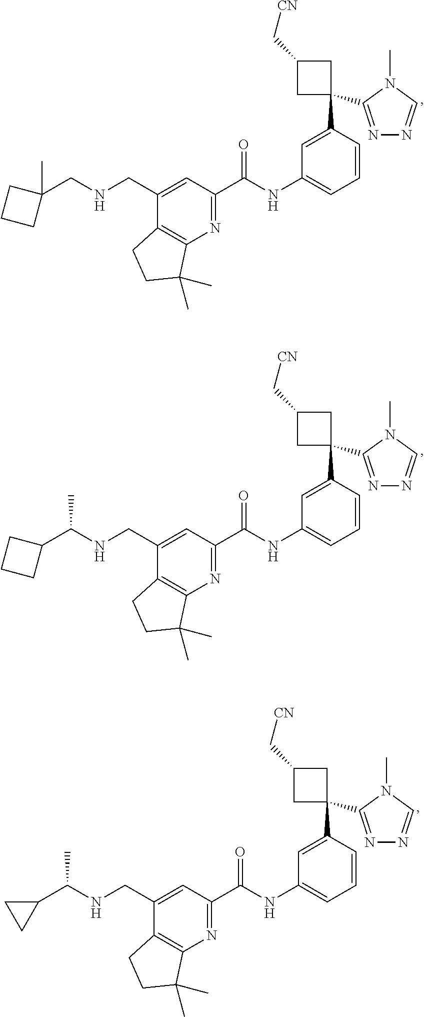 [SMILES: Cn1cnnc1[C@]1(c2cccc(NC(=O)c3cc(CNCC4(C)CCC4)c4c(n3)C(C)(C)CC4)c2)C[C@H](CC#N)C1 ; C[C@H](NCc1cc(C(=O)Nc2cccc([C@]3(c4nncn4C)C[C@@H](CC#N)C3)c2)nc2c1CCC2(C)C)C1CCC1 ; C[C@H](NCc1cc(C(=O)Nc2cccc([C@]3(c4nncn4C)C[C@@H](CC#N)C3)c2)nc2c1CCC2(C)C)C1CC1] 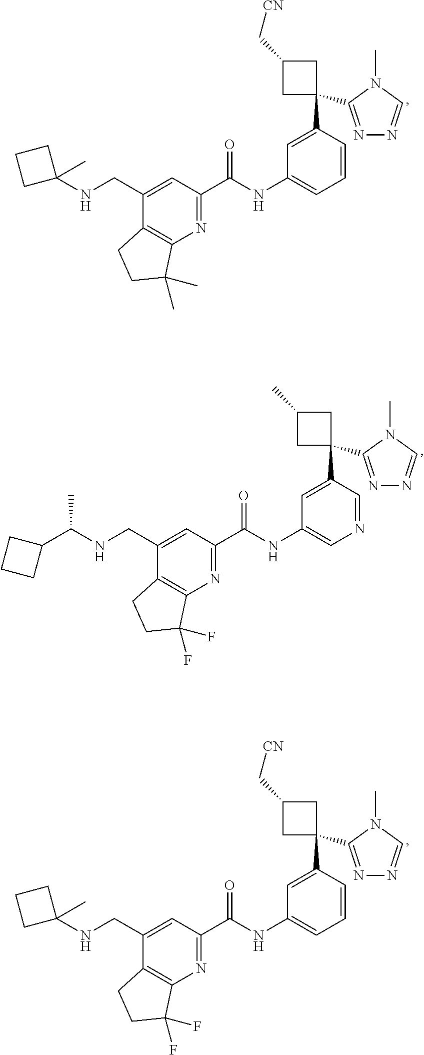 [SMILES: Cn1cnnc1[C@]1(c2cccc(NC(=O)c3cc(CNC4(C)CCC4)c4c(n3)C(C)(C)CC4)c2)C[C@H](CC#N)C1 ; C[C@H](NCc1cc(C(=O)Nc2cncc([C@]3(c4nncn4C)C[C@@H](C)C3)c2)nc2c1CCC2(F)F)C1CCC1 ; Cn1cnnc1[C@]1(c2cccc(NC(=O)c3cc(CNC4(C)CCC4)c4c(n3)C(F)(F)CC4)c2)C[C@H](CC#N)C1] 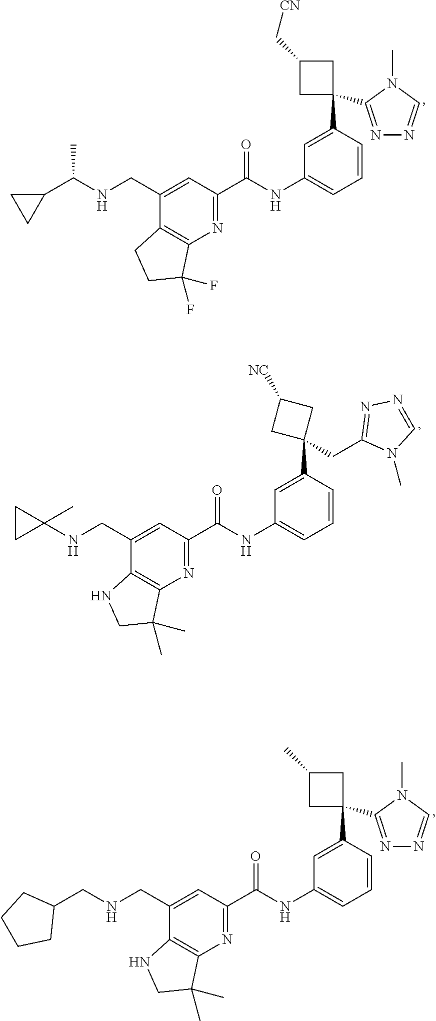 [SMILES: C[C@H](NCc1cc(C(=O)Nc2cccc([C@]3(c4nncn4C)C[C@@H](CC#N)C3)c2)nc2c1CCC2(F)F)C1CC1 ; Cn1cnnc1C[C@]1(c2cccc(NC(=O)c3cc(CNC4(C)CC4)c4c(n3)C(C)(C)CN4)c2)C[C@H](C#N)C1 ; Cn1cnnc1[C@]1(c2cccc(NC(=O)c3cc(CNCC4CCCC4)c4c(n3)C(C)(C)CN4)c2)C[C@H](C)C1] 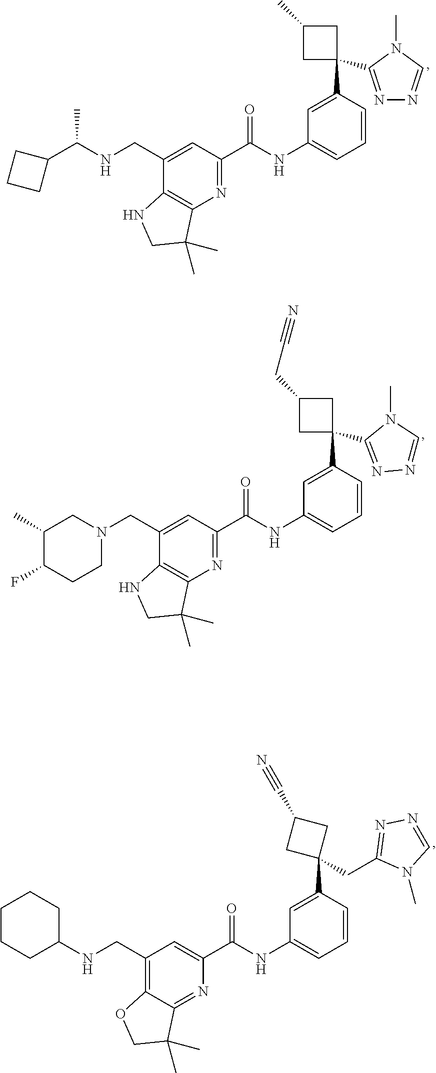 [SMILES: C[C@H](NCc1cc(C(=O)Nc2cccc([C@]3(c4nncn4C)C[C@@H](C)C3)c2)nc2c1NCC2(C)C)C1CCC1 ; C[C@@H]1CN(Cc2cc(C(=O)Nc3cccc([C@]4(c5nncn5C)C[C@@H](CC#N)C4)c3)nc3c2NCC3(C)C)CC[C@@H]1F ; Cn1cnnc1C[C@]1(c2cccc(NC(=O)c3cc(CNC4CCCCC4)c4c(n3)C(C)(C)CO4)c2)C[C@H](C#N)C1] 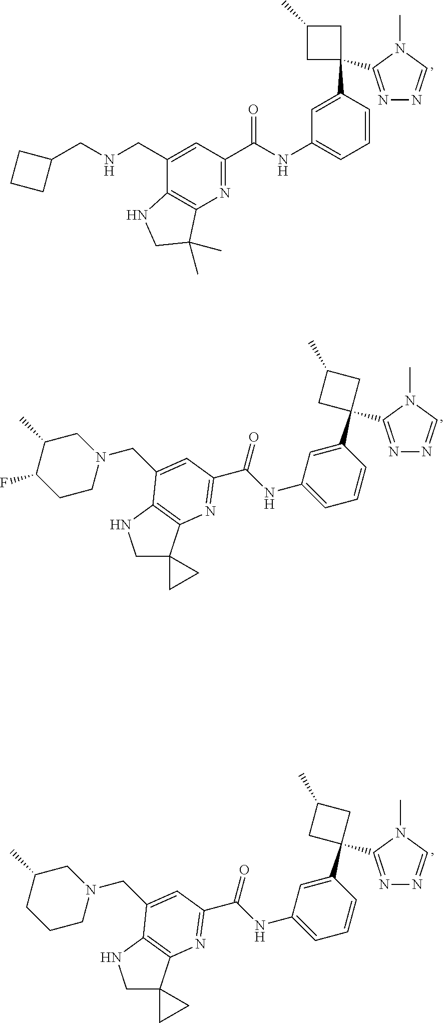 [SMILES: Cn1cnnc1[C@]1(c2cccc(NC(=O)c3cc(CNCC4CCC4)c4c(n3)C(C)(C)CN4)c2)C[C@H](C)C1 ; C[C@@H]1CN(Cc2cc(C(=O)Nc3cccc([C@]4(c5nncn5C)C[C@@H](C)C4)c3)nc3c2NCC32CC2)CC[C@@H]1F ; C[C@H]1CCCN(Cc2cc(C(=O)Nc3cccc([C@]4(c5nncn5C)C[C@@H](C)C4)c3)nc3c2NCC32CC2)C1] 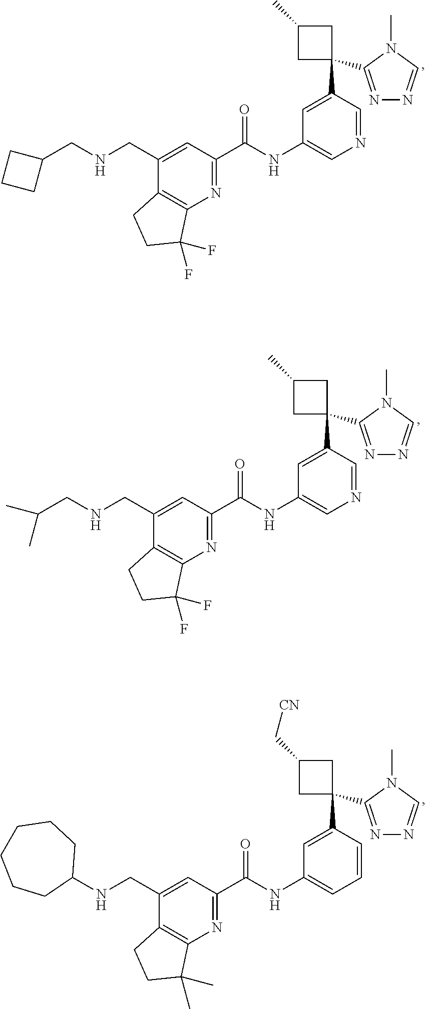 [SMILES: Cn1cnnc1[C@]1(c2cncc(NC(=O)c3cc(CNCC4CCC4)c4c(n3)C(F)(F)CC4)c2)C[C@H](C)C1 ; CC(C)CNCc1cc(C(=O)Nc2cncc([C@]3(c4nncn4C)C[C@@H](C)C3)c2)nc2c1CCC2(F)F ; Cn1cnnc1[C@]1(c2cccc(NC(=O)c3cc(CNC4CCCCCC4)c4c(n3)C(C)(C)CC4)c2)C[C@H](CC#N)C1] 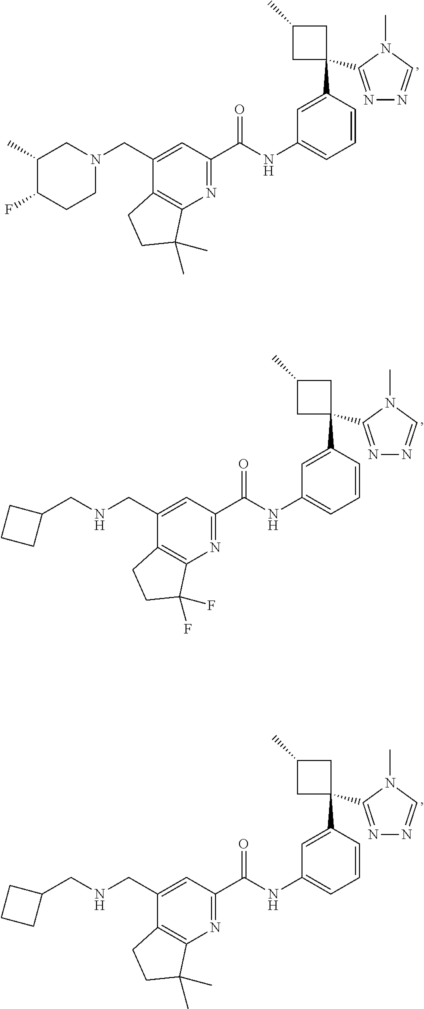 [SMILES: C[C@@H]1CN(Cc2cc(C(=O)Nc3cccc([C@]4(c5nncn5C)C[C@@H](C)C4)c3)nc3c2CCC3(C)C)CC[C@@H]1F ; Cn1cnnc1[C@]1(c2cccc(NC(=O)c3cc(CNCC4CCC4)c4c(n3)C(F)(F)CC4)c2)C[C@H](C)C1 ; Cn1cnnc1[C@]1(c2cccc(NC(=O)c3cc(CNCC4CCC4)c4c(n3)C(C)(C)CC4)c2)C[C@H](C)C1] 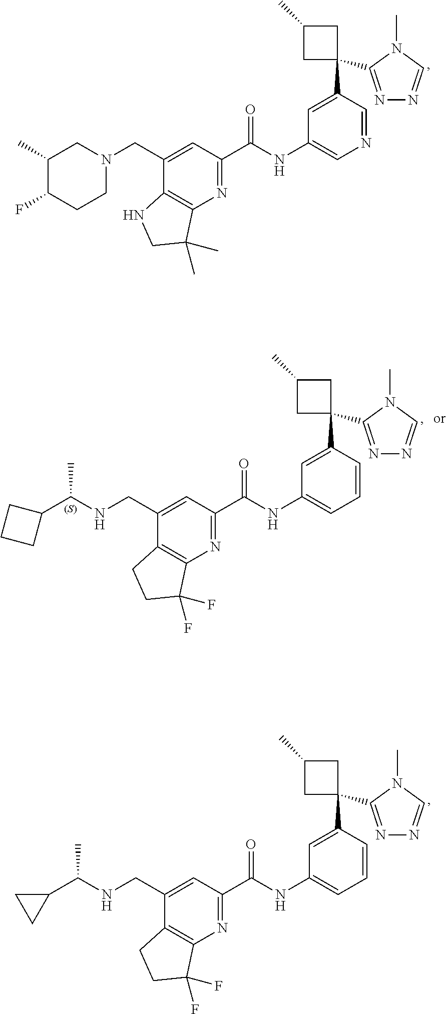
or a pharmaceutically acceptable salt thereof.

51. A pharmaceutical composition comprising an effective amount of a compound of claim 1, or a pharmaceutically acceptable salt, tautomer, isotopologue, stereoisomer, or enantiomer thereof, and a pharmaceutically acceptable carrier, excipient or vehicle.

52. (canceled)

53. A method for the treatment or prevention of a cancer responsive to Cbl-b activity, the method comprising administering to a subject in need thereof an effective amount of a compound of claim 1, or a pharmaceutically acceptable salt, tautomer, isotopologue, stereoisomer, or enantiomer thereof.

54-55. (canceled)
